(19) United States
(12) Patent Application Publication
(10) Pub. No.: US 2023/0174933 A1
(43) Pub. Date: Jun. 8, 2023

(54) METHODS OF MAKING CHIMERIC ANTIGEN RECEPTOR-EXPRESSING CELLS

(71) Applicant: Novartis AG, Basel (CH)

(72) Inventors: Jennifer BROGDON, Sudbury, MA (US); Seth CARBONNEAU, Roxbury, MA (US); Glenn DRANOFF, Sudbury, MA (US); Michael R. Greene, Groton, MA (US); Anniesha HACK, East Hanover, NJ (US); Marc Horst Peter HILD, Wellesley, MA (US); Olja KODRASI, Quincy, MA (US); Elizabeth Dorothy PRATICO, East Boston, MA (US); Andrew PRICE, Cambridge, MA (US); Andrew Marc STEIN, Cambridge, MA (US); Attilio BONDANZA, Basel (CH); Boris ENGELS, Arlington, MA (US); Carla Patricia Pinto GUIMARAES, Boston, MA (US); Hyungwook LIM, Cambridge, MA (US); Sujata SHARMA, Ashland, MA (US); Akash SOHONI, Cambridge, MA (US); Louise TREANOR, Cambridge, MA (US); Xu ZHU, Cambridge, MA (US)

(21) Appl. No.: 17/801,665

(22) PCT Filed: Feb. 26, 2021
(86) PCT No.: PCT/US2021/019904

§ 371 Date: Aug. 23, 2022

Related U.S. Application Data
(60) Provisional application No. 62/982,698, filed on Feb. 27, 2020.

(51) Int. Cl. C12N 5/0783; A61K 35/17; A61P 35/00; C07K 16/28; C07K 14/705; C07K 14/725; C07K 14/47; C07K 14/71; A61K 38/17; A61K 39/395; A61K 45/06; A61P 35/02

(52) U.S. Cl. CPC C12N 5/0636; A61K 35/17; A61P 35/00; C07K 16/2878; C07K 14/70517; C07K 14/7051; C07K 14/70578; C07K 14/4702; C07K 14/71; A61K 38/1774; A61K 38/177; A61K 38/1709; A61K 38/179; A61K 39/39558; A61K 45/06; C07K 16/2803; A61P 35/02; C07K 2319/33; A61K 2039/505

(57) ABSTRACT

This disclosure provides methods of making immune effector cells (for example, T cells, NK cells) that comprise (i) a nucleic acid molecule that encodes a controllable chimeric antigen receptor (CCAR) or (ii) a nucleic acid molecule that encodes a CAR and a regulatory molecule, and compositions generated by such methods.

### RELATED APPLICATIONS

This application claims priority to U.S. Provisional Application 62/982,698 filed on Feb. 27, 2020, the entire contents of which are hereby incorporated by reference.

### SEQUENCE LISTING

The instant application contains a Sequence Listing which has been submitted electronically in ASCII format and is hereby incorporated by reference in its entirety. Said ASCII copy, created on Feb. 19, 2021, is named N2067-7169WO_SL.txt and is 386,964 bytes in size.

### FIELD OF THE INVENTION

The present disclosure relates generally to methods of making immune effector cells (for example, T cells or NK cells) engineered to express a Chimeric Antigen Receptor (CAR), and compositions comprising the same.

### BACKGROUND OF THE INVENTION

Adoptive cell transfer (ACT) therapy with T cells, especially with T cells transduced with Chimeric Antigen Receptors (CARs), has shown promise in several hematologic cancer trials. The manufacture of gene-modified T cells is currently a complex process. There exists a need for methods and processes to improve production of the CAR-expressing cell therapy product, enhance product quality, and maximize the therapeutic efficacy of the product.

### SUMMARY OF THE INVENTION

The present disclosure pertains to methods of making immune effector cells (for example, T cells or NK cells) engineered to express a CAR, and compositions generated using such methods. Also disclosed are methods of using such compositions for treating a disease, for example, cancer, in a subject.

In some embodiments, this disclosure features a method of making a population of cells (for example, T cells) that comprise: a first nucleic acid molecule that encodes a controllable chimeric antigen receptor (CCAR), or a second nucleic acid molecule that encodes a chimeric antigen receptor (CAR) and a regulatory molecule. In some embodiments, this disclosure features a method of making a population of cells (for example, T cells) that comprise a first nucleic acid molecule that encodes a controllable chimeric antigen receptor (CCAR). In some embodiments, this disclosure features a method of making a population of cells (for example, T cells) that comprise a second nucleic acid molecule that encodes a chimeric antigen receptor (CAR) and a regulatory molecule. In some embodiments, the second nucleic acid molecule comprises one or more nucleic acid molecules, e.g., the second nucleic acid molecule comprises a third nucleic acid molecule and a fourth nucleic acid molecule, wherein the third nucleic acid molecule comprises a nucleic acid sequence encoding the CAR and the fourth nucleic acid molecule comprises a nucleic acid sequence encoding the regulatory molecule.

In some embodiments, the method comprises: (i) contacting (for example, binding) a population of cells (for example, T cells, for example, T cells isolated from a frozen or fresh leukapheresis product) with an agent that stimulates a CD3/TCR complex and/or an agent that stimulates a costimulatory molecule on the surface of the cells; (ii) contacting the population of cells (for example, T cells) with a first nucleic acid molecule (for example, a DNA or RNA molecule) encoding a CCAR or a second nucleic acid molecule (for example, a DNA or RNA molecule) encoding a CAR and a regulatory molecule, thereby providing a population of cells (for example, T cells) comprising the first or second nucleic acid molecule, and (iii) harvesting the population of cells (for example, T cells) for storage (for example, reformulating the population of cells in cryopreservation media) or administration. In some embodiments, step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and step (iii) is performed no later than 30 (for example, 26) hours after the beginning of step (i), for example, no later than 22, 23, 24, 25, 26, 27, 28, 29, or 30 hours after the beginning of step (i), for example, no later than 24 hours after the beginning of step (i). In some embodiments, step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and step (iii) is performed no later than 30 hours after the beginning of step (ii), for example, no later than 22, 23, 24, 25, 26, 27, 28, 29, or 30 hours after the beginning of step (ii). In some embodiments, the population of cells from step (iii) are not expanded, or expanded by no more than 5, 10, 15, 20, 25, 30, 35, or 40%, for example, no more than 10%, for example, as assessed by the number of living cells, compared to the population of cells at the beginning of step (i). In some embodiments, the first or second nucleic acid molecule in step (ii) is on a viral vector. In some embodiments, the first or second nucleic acid molecule in step (ii) is an RNA molecule on a viral vector. In some embodiments, step (ii) comprises transducing the population of cells (for example, T cells) with a viral vector comprising the first or second nucleic acid molecule.

In some embodiments, the agent that stimulates a CD3/TCR complex is an agent that stimulates CD3 (for example, an anti-CD3 antibody). In some embodiments, the agent that stimulates a costimulatory molecule is an agent that stimulates CD28, ICOS, CD27, HVEM, LIGHT, CD40, 4-1BB, OX40, DR3, GITR, CD30, TIM1, CD2, CD226, or any combination thereof. In some embodiments, the agent that stimulates a CD3/TCR complex or the agent that stimulates a costimulatory molecule is chosen from an antibody (for example, a single-domain antibody (for example, a heavy chain variable domain antibody), a peptibody, a Fab fragment, or a scFv), a small molecule, or a ligand (for example, a naturally-existing, recombinant, or chimeric ligand). In some embodiments, the agent that stimulates a CD3/TCR complex or the agent that stimulates a costimulatory molecule does not comprise a bead. In some embodiments, the agent that stimulates a CD3/TCR complex comprises an anti-CD3 antibody and the agent that stimulates a costimulatory molecule comprises an anti-CD28 antibody. In some embodiments, the agent that stimulates a CD3/TCR complex comprises an anti-CD3 antibody covalently attached to a colloidal polymeric nanomatrix and the agent that stimulates a costimulatory molecule comprises an anti-CD28 antibody covalently attached to a colloidal polymeric nanomatrix. In some embodiments, the agent that stimulates a CD3/TCR complex and the agent that stimulates a costimulatory molecule comprise T Cell TransAct™.

In some embodiments, step (i) increases the percentage of cells that comprise the first or second nucleic acid molecule in the population of cells from step (iii). In some embodiments, the population of cells from step (iii) shows a higher percentage of cells that comprise the first or second nucleic acid molecule (for example, at least 10, 20, 30, 40, 50, or 60% higher), compared with cells made by an otherwise similar method without step (i).

In some embodiments, the percentage of naïve cells, for example, naïve T cells, for example, CD45RA+ CD45RO− CCR7+ T cells, in the population of cells from step (iii) is the same as or differs by no more than 5 or 10% from the percentage of naïve cells, for example, naïve T cells, for example, CD45RA+ CD45RO− CCR7+ cells, in the population of cells at the beginning of step (i). In some embodiments, the percentage of naïve cells, for example, naïve T cells, for example, CD45RA+ CD45RO− CCR7+ T cells, in the population of cells from step (iii) is increased by, for example, at least 1.2, 1.4, 1.6, 1.8, 2.0, 2.2, 2.4, 2.6, 2.8, or 3-fold, as compared to the percentage of naïve cells, for example, naïve T cells, for example, CD45RA+ CD45RO− CCR7+ cells, in the population of cells at the beginning of step (i). In some embodiments, the percentage of naïve T cells that comprise the first or second nucleic acid molecule, for example, CD45RA+ CD45RO− CCR7+ T cells that comprise the first or second nucleic acid molecule, in the population of cells increases during the duration of step (ii), for example, increases by, for example, at least 30, 35, 40, 45, 50, 55, or 60%, between 18-24 hours after the beginning of step (ii). In some embodiments, the percentage of naïve cells, for example, naïve T cells, for example, CD45RA+ CD45RO− CCR7+ T cells, in the population of cells from step (iii) does not decrease, or decreases by no more than 5 or 10%, as compared to the percentage of naïve cells, for example, naïve T cells, for example, CD45RA+ CD45RO− CCR7+ cells, in the population of cells at the beginning of step (i).

In some embodiments, the population of cells from step (iii) shows a higher percentage of naïve cells, for example, naïve T cells, for example, CD45RA+ CD45RO− CCR7+ T cells (for example, at least 10, 20, 30, or 40% higher), compared with cells made by an otherwise similar method in which step (iii) is performed more than 26 hours after the beginning of step (i), for example, more than 5, 6, 7, 8, 9, 10, 11, or 12 days after the beginning of step (i). In some embodiments, the percentage of naïve cells, for example, naïve T cells, for example, CD45RA+ CD45RO− CCR7+ T cells, in the population of cells from step (iii) is higher (for example, at least 1.2, 1.4, 1.6, 1.8, 2.0, 2.2, 2.4, 2.6, 2.8, or 3-fold higher) than the percentage of naïve cells, for example, naïve T cells, for example, CD45RA+ CD45RO− CCR7+ T cells, in cells made by an otherwise similar method in which step (iii) is performed more than 26 hours after the beginning of step (i), for example, more than 5, 6, 7, 8, 9, 10, 11, or 12 days after the beginning of step (i). In some embodiments, the percentage of naïve T cells that comprise the first or second nucleic acid molecule, for example, CD45RA+ CD45RO− CCR7+ T cells that comprise the first or second nucleic acid molecule, in the population of cells from step (iii) is higher (for example, at least 4, 6, 8, 10, or 12-fold higher) than the percentage of naïve T cells that comprise the first or second nucleic acid molecule, for example, CD45RA+ CD45RO− CCR7+ T cells that comprise the first or second nucleic acid molecule, in cells made by an otherwise similar method in which step (iii) is performed more than 26 hours after the beginning of step (i), for example, more than 5, 6, 7, 8, 9, 10, 11, or 12 days after the beginning of step (i). In some embodiments, the population of cells from step (iii) shows a higher percentage of naïve cells, for example, naïve T cells, for example, CD45RA+ CD45RO− CCR7+ T cells (for example, at least 10, 20, 30, or 40% higher), compared with cells made by an otherwise similar method which further comprises, after step (ii) and prior to step (iii), expanding the population of cells (for example, T cells) in vitro for more than 3 days, for example, for 5, 6, 7, 8 or 9 days. In some embodiments, the percentage of naïve cells, for example, naïve T cells, for example, CD45RA+ CD45RO− CCR7+ T cells, in the population of cells from step (iii) is higher (for example, at least 1.2, 1.4, 1.6, 1.8, 2.0, 2.2, 2.4, 2.6, 2.8, or 3-fold higher) than the percentage of naïve cells, for example, naïve T cells, for example, CD45RA+ CD45RO− CCR7+ T cells, in cells made by an otherwise similar method which further comprises, after step (ii) and prior to step (iii), expanding the population of cells (for example, T cells) in vitro for more than 3 days, for example, for 5, 6, 7, 8 or 9 days. In some embodiments, the percentage of naïve T cells that comprise the first or second nucleic acid molecule, for example, CD45RA+ CD45RO− CCR7+ T cells that comprise the first or second nucleic acid molecule, in the population of cells from step (iii) is higher (for example, at least 4, 6, 8, 10, or 12-fold higher) than the percentage of naïve T cells that comprise the first or second nucleic acid molecule, for example, CD45RA+ CD45RO− CCR7+ T cells that comprise the first or second nucleic acid molecule, in cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the percentage of central memory cells, for example, central memory T cells, for example, CD95+ central memory T cells, in the population of cells from step (iii) is the same as or differs by no more than 5 or 10% from the percentage of central memory cells, for example, central memory T cells, for example, CD95+ central memory T cells, in the population of cells at the beginning of step (i). In some embodiments, the percentage of central memory cells, for example, central memory T cells, for example, CCR7+CD45RO+ T cells, in the population of cells from step (iii) is reduced by at least 20, 25, 30, 35, 40, 45, or 50%, as compared to the percentage of central memory cells, for example, central memory T cells, for example, CCR7+CD45RO+ T cells, in the population of cells at the beginning of step (i). In some embodiments, the percentage of central memory T cells that comprise the first or second nucleic acid molecule, for example, CCR7+CD45RO+ cells that comprise the first or second nucleic acid molecule, decreases during the duration of step (ii), for example, decreases by, for example, at least 8, 10, 12, 14, 16, 18, or 20%, between 18-24 hours after the beginning of step (ii). In some embodiments, the percentage of central memory cells, for example, central memory T cells, for example, CCR7+CD45RO+ T cells, in the population of cells from step (iii) does not increase, or increases by no more than 5 or 10%, as compared to the percentage of central memory cells, for example, central memory T cells, for example, CCR7+CD45RO+ T cells, in the population of cells at the beginning of step (i).

In some embodiments, the population of cells from step (iii) shows a lower percentage of central memory cells, for example, central memory T cells, for example, CD95+ central memory T cells (for example, at least 10, 20, 30, or 40% lower), compared with cells made by an otherwise similar method in which step (iii) is performed more than 26 hours after the beginning of step (i), for example, more than 5, 6, 7, 8, 9, 10, 11, or 12 days after the beginning of step (i). In some embodiments, the percentage of central memory cells, for example, central memory T cells, for example, CCR7+CD45RO+ T cells in the population of cells from step (iii) is lower (for example, at least 20, 30, 40, or 50% lower) than the percentage of central memory cells, for example, central memory T cells, for example, CCR7+CD45RO+ T cells, in cells made by an otherwise similar method in which step (iii) is performed more than 26 hours after the beginning of step (i), for example, more than 5, 6, 7, 8, 9, 10, 11, or 12 days after the beginning of step (i). In some embodiments, the percentage of central memory T cells that comprise the first or second nucleic acid molecule, for example, CCR7+CD45RO+ T cells that comprise the first or second nucleic acid molecule, in the population of cells from step (iii) is lower (for example, at least 10, 20, 30, or 40% lower) than the percentage of central memory T cells that comprise the first or second nucleic acid molecule, for example, CCR7+CD45RO+ T cells that comprise the first or second nucleic acid molecule, in cells made by an otherwise similar method in which step (iii) is performed more than 26 hours after the beginning of step (i), for example, more than 5, 6, 7, 8, 9, 10, 11, or 12 days after the beginning of step (i). In some embodiments, the population of cells from step (iii) shows a lower percentage of central memory cells, for example, central memory T cells, for example, CD95+ central memory T cells (for example, at least 10, 20, 30, or 40% lower), compared with cells made by an otherwise similar method which further comprises, after step (ii) and prior to step (iii), expanding the population of cells (for example, T cells) in vitro for more than 3 days, for example, for 5, 6, 7, 8 or 9 days. In some embodiments, the percentage of central memory cells, for example, central memory T cells, for example, CCR7+CD45RO+ T cells in the population of cells from step (iii) is lower (for example, at least 20, 30, 40, or 50% lower) than the percentage of central memory cells, for example, central memory T cells, for example, CCR7+CD45RO+ T cells, in cells made by an otherwise similar method which further comprises, after step (ii) and prior to step (iii), expanding the population of cells (for example, T cells) in vitro for more than 3 days, for example, for 5, 6, 7, 8 or 9 days. In some embodiments, the percentage of central memory T cells that comprise the first or second nucleic acid molecule, for example, CCR7+CD45RO+ T cells that comprise the first or second nucleic acid molecule, in the population of cells from step (iii) is lower (for example, at least 10, 20, 30, or 40% lower) than the percentage of central memory T cells that comprise the first or second nucleic acid molecule, for example, CCR7+CD45RO+ T cells that comprise the first or second nucleic acid molecule, in cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the percentage of stem memory T cells, for example, CD45RA+CD95+IL-2 receptor β+CCR7+CD62L+ T cells, in the population of cells from step (iii) is increased, as compared to the percentage of stem memory T cells, for example, CD45RA+CD95+IL-2 receptor β+CCR7+CD62L+ T cells, in the population of cells at the beginning of step (i). In some embodiments, the percentage of stem memory T cells that comprise the first or second nucleic acid molecule, for example, CD45RA+CD95+IL-2 receptor β+CCR7+CD62L+ T cells that comprise the first or second nucleic acid molecule, in the population of cells from step (iii) is increased, as compared to the percentage of stem memory T cells that comprise the first or second nucleic acid molecule, for example, CD45RA+CD95+IL-2 receptor β+CCR7+CD62L+ T cells that comprise the first or second nucleic acid molecule, in the population of cells at the beginning of step (i). In some embodiments, the percentage of stem memory T cells, for example, CD45RA+CD95+IL-2 receptor β+CCR7+CD62L+ T cells, in the population of cells from step (iii) is higher than the percentage of stem memory T cells, for example, CD45RA+CD95+IL-2 receptor β+CCR7+CD62L+ T cells, in cells made by an otherwise similar method in which step (iii) is performed more than 26 hours after the beginning of step (i), for example, more than 5, 6, 7, 8, 9, 10, 11, or 12 days after the beginning of step (i). In some embodiments, the percentage of stem memory T cells that comprise the first or second nucleic acid molecule, for example, CD45RA+CD95+IL-2 receptor β+CCR7+CD62L+ T cells that comprise the first or second nucleic acid molecule, in the population of cells from step (iii) is higher than the percentage of stem memory T cells that comprise the first or second nucleic acid molecule, for example, CD45RA+CD95+IL-2 receptor β+CCR7+CD62L+ T cells that comprise the first or second nucleic acid molecule, in cells made by an otherwise similar method in which step (iii) is performed more than 26 hours after the beginning of step (i), for example, more than 5, 6, 7, 8, 9, 10, 11, or 12 days after the beginning of step (i). In some embodiments, the percentage of stem memory T cells, for example, CD45RA+CD95+IL-2 receptor β+CCR7+CD62L+ T cells, in the population of cells from step (iii) is higher than the percentage of stem memory T cells, for example, CD45RA+CD95+IL-2 receptor β+CCR7+CD62L+ T cells, in cells made by an otherwise similar method which further comprises, after step (ii) and prior to step (iii), expanding the population of cells (for example, T cells) in vitro for more than 3 days, for example, for 5, 6, 7, 8 or 9 days. In some embodiments, the percentage of stem memory T cells that comprise the first or second nucleic acid molecule, for example, CD45RA+CD95+IL-2 receptor β+CCR7+CD62L+ T cells that comprise the first or second nucleic acid molecule, in the population of cells from step (iii) is higher than the percentage of stem memory T cells that comprise the first or second nucleic acid molecule, for example, CD45RA+CD95+IL-2 receptor β+CCR7+CD62L+ T cells that comprise the first or second nucleic acid molecule, in cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the median GeneSetScore (Up TEM vs. Down TSCM) of the population of cells from step (iii) is about the same as or differs by no more than (for example, increased by no more than) about 25, 50, 75, 100, or 125% from the median GeneSetScore (Up TEM vs. Down TSCM) of the population of cells at the beginning of step (i). In some embodiments, the median GeneSetScore (Up TEM vs. Down TSCM) of the population of cells from step (iii) is lower (for example, at least about 100, 150, 200, 250, or 300% lower) than the median GeneSetScore (Up TEM vs. Down TSCM) of: cells made by an otherwise similar method in which step (iii) is performed more than 26 hours after the beginning of step (i), for example, more than 5, 6, 7, 8, 9, 10, 11, or 12 days after the beginning of step (i), or cells made by an otherwise similar method which further comprises, after step (ii) and prior to step (iii), expanding the population of cells (for example, T cells) in vitro for more than 3 days, for example, for 5, 6, 7, 8 or 9 days. In some embodiments, the median GeneSetScore (Up Treg vs. Down Teff) of the population of cells from step (iii) is about the same as or differs by no more than (for example, increased by no more than) about 25, 50, 100, 150, or 200% from the median GeneSetScore (Up Treg vs. Down Teff) of the population of cells at the beginning of step (i). In some embodiments, the median GeneSetScore (Up Treg vs. Down Teff) of the population of cells from step (iii) is lower (for example, at least about 50, 100, 125, 150, or 175% lower) than the median GeneSetScore (Up Treg vs. Down Teff) of: cells made by an otherwise similar method in which step (iii) is performed more than 26 hours after the beginning of step (i), for example, more than 5, 6, 7, 8, 9, 10, 11, or 12 days after the beginning of step (i), or cells made by an otherwise similar method which further comprises, after step (ii) and prior to step (iii), expanding the population of cells (for example, T cells) in vitro for more than 3 days, for example, for 5, 6, 7, 8 or 9 days. In some embodiments, the median GeneSetScore (Down stemness) of the population of cells from step (iii) is about the same as or differs by no more than (for example, increased by no more than) about 25, 50, 100, 150, 200, or 250% from the median GeneSetScore (Down stemness) of the population of cells at the beginning of step (i). In some embodiments, the median GeneSetScore (Down stemness) of the population of cells from step (iii) is lower (for example, at least about 50, 100, or 125% lower) than the median GeneSetScore (Down stemness) of: cells made by an otherwise similar method in which step (iii) is performed more than 26 hours after the beginning of step (i), for example, more than 5, 6, 7, 8, 9, 10, 11, or 12 days after the beginning of step (i), or cells made by an otherwise similar method which further comprises, after step (ii) and prior to step (iii), expanding the population of cells (for example, T cells) in vitro for more than 3 days, for example, for 5, 6, 7, 8 or 9 days. In some embodiments, the median GeneSetScore (Up hypoxia) of the population of cells from step (iii) is about the same as or differs by no more than (for example, increased by no more than) about 125, 150, 175, or 200% from the median GeneSetScore (Up hypoxia) of the population of cells at the beginning of step (i). In some embodiments, the median GeneSetScore (Up hypoxia) of the population of cells from step (iii) is lower (for example, at least about 40, 50, 60, 70, or 80% lower) than the median GeneSetScore (Up hypoxia) of: cells made by an otherwise similar method in which step (iii) is performed more than 26 hours after the beginning of step (i), for example, more than 5, 6, 7, 8, 9, 10, 11, or 12 days after the beginning of step (i), or cells made by an otherwise similar method which further comprises, after step (ii) and prior to step (iii), expanding the population of cells (for example, T cells) in vitro for more than 3 days, for example, for 5, 6, 7, 8 or 9 days. In some embodiments, the median GeneSetScore (Up autophagy) of the population of cells from step (iii) is about the same as or differs by no more than (for example, increased by no more than) about 180, 190, 200, or 210% from the median GeneSetScore (Up autophagy) of the population of cells at the beginning of step (i). In some embodiments, the median GeneSetScore (Up autophagy) of the population of cells from step (iii) is lower (for example, at least 20, 30, or 40% lower) than the median GeneSetScore (Up autophagy) of: cells made by an otherwise similar method in which step (iii) is performed more than 26 hours after the beginning of step (i), for example, more than 5, 6, 7, 8, 9, 10, 11, or 12 days after the beginning of step (i), or cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the population of cells from step (iii), after being incubated with a cell expressing an antigen recognized by the CCAR or CAR, secretes IL-2 at a higher level (for example, at least 2, 4, 6, 8, 10, 12, or 14-fold higher) than cells made by an otherwise similar method in which step (iii) is performed more than 26 hours after the beginning of step (i), for example, more than 5, 6, 7, 8, 9, 10, 11, or 12 days after the beginning of step (i), or cells made by an otherwise similar method which further comprises, after step (ii) and prior to step (iii), expanding the population of cells (for example, T cells) in vitro for more than 3 days, for example, for 5, 6, 7, 8 or 9 days, for example, as assessed using methods described in Example 8 with respect to FIGS. 29C-29D.

In some embodiments, the population of cells from step (iii), after being administered in vivo, persists longer or expands at a higher level (for example, as assessed using methods described in Example 1 with respect to FIG. 4C), compared with cells made by an otherwise similar method in which step (iii) is performed more than 26 hours after the beginning of step (i), for example, more than 5, 6, 7, 8, 9, 10, 11, or 12 days after the beginning of step (i), or compared with cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the population of cells from step (iii), after being administered in vivo, shows a stronger anti-tumor activity (for example, a stronger anti-tumor activity at a low dose, for example, a dose no more than 0.15×10⁶, 0.2×10⁶, 0.25×10⁶, or 0.3×10⁶ viable cells that comprise the first or second nucleic acid molecule) than cells made by an otherwise similar method in which step (iii) is performed more than 26 hours after the beginning of step (i), for example, more than 5, 6, 7, 8, 9, 10, 11, or 12 days after the beginning of step (i), or cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the population of cells from step (iii) are not expanded, or expanded by no more than 5, 10, 15, 20, 25, 30, 35, or 40%, for example, no more than 10%, for example, as assessed by the number of living cells, compared to the population of cells at the beginning of step (i), optionally wherein the number of living cells in the population of cells from step (iii) decreases from the number of living cells in the population of cells at the beginning of step (i).

In some embodiments, the population of cells from step (iii) are not expanded, or expanded by less than 2 hours, for example, less than 1 or 1.5 hours, compared to the population of cells at the beginning of step (i).

In some embodiments, steps (i) and/or (ii) are performed in cell media (for example, serum-free media) comprising IL-2, IL-15 (for example, hetIL-15 (IL15/sIL-15Ra)), IL-7, IL-21, IL-6 (for example, IL-6/sIL-6Ra), a LSD1 inhibitor, a MALT1 inhibitor, or a combination thereof.

In some embodiments, steps (i) and/or (ii) are performed in serum-free cell media comprising a serum replacement. In some embodiments, the serum replacement is CTSTM Immune Cell Serum Replacement (ICSR).

In some embodiments, the method further comprises prior to step (i): (iv) (optionally) receiving a fresh leukapheresis product (or an alternative source of hematopoietic tissue such as a fresh whole blood product, a fresh bone marrow product, or a fresh tumor or organ biopsy or removal (for example, a fresh product from thymectomy)) from an entity, for example, a laboratory, hospital, or healthcare provider, and (v) isolating the population of cells (for example, T cells, for example, CD8+ and/or CD4+ T cells) contacted in step (i) from a fresh leukapheresis product (or an alternative source of hematopoietic tissue such as a fresh whole blood product, a fresh bone marrow product, or a fresh tumor or organ biopsy or removal (for example, a fresh product from thymectomy)). In some embodiments, step (iii) is performed no later than 35 hours after the beginning of step (v), for example, no later than 27, 28, 29, 30, 31, 32, 33, 34, or 35 hours after the beginning of step (v), for example, no later than 30 hours after the beginning of step (v). In some embodiments, the population of cells from step (iii) are not expanded, or expanded by no more than 5, 10, 15, 20, 25, 30, 35, or 40%, for example, no more than 10%, for example, as assessed by the number of living cells, compared to the population of cells at the end of step (v).

In some embodiments, the method further comprises prior to step (i): receiving cryopreserved T cells isolated from a leukapheresis product (or an alternative source of hematopoietic tissue such as cryopreserved T cells isolated from whole blood, bone marrow, or tumor or organ biopsy or removal (for example, thymectomy)) from an entity, for example, a laboratory, hospital, or healthcare provider.

In some embodiments, the method further comprises prior to step (i): (iv) (optionally) receiving a cryopreserved leukapheresis product (or an alternative source of hematopoietic tissue such as a cryopreserved whole blood product, a cryopreserved bone marrow product, or a cryopreserved tumor or organ biopsy or removal (for example, a cryopreserved product from thymectomy)) from an entity, for example, a laboratory, hospital, or healthcare provider, and (v) isolating the population of cells (for example, T cells, for example, CD8+ and/or CD4+ T cells) contacted in step (i) from a cryopreserved leukapheresis product (or an alternative source of hematopoietic tissue such as a cryopreserved whole blood product, a cryopreserved bone marrow product, or a cryopreserved tumor or organ biopsy or removal (for example, a cryopreserved product from thymectomy)). In some embodiments, step (iii) is performed no later than 35 hours after the beginning of step (v), for example, no later than 27, 28, 29, 30, 31, 32, 33, 34, or 35 hours after the beginning of step (v), for example, no later than 30 hours after the beginning of step (v). In some embodiments, the population of cells from step (iii) are not expanded, or expanded by no more than 5, 10, 15, 20, 25, 30, 35, or 40%, for example, no more than 10%, for example, as assessed by the number of living cells, compared to the population of cells at the end of step (v).

In some embodiments, the method further comprises step (vi): culturing a portion of the population of cells from step (iii) for at least 2, 2.5, 3, 3.5, 4, 4.5, 5, 5.5, 6, 6.5, or 7 days, for example, at least 2 days and no more than 7 days, and measuring CAR expression level in the portion (for example, measuring the percentage of viable, CAR-expressing cells in the portion). In some embodiments, step (iii) comprises harvesting and freezing the population of cells (for example, T cells) and step (vi) comprises thawing a portion of the population of cells from step (iii), culturing the portion for at least 2, 2.5, 3, 3.5, 4, 4.5, 5, 5.5, 6, 6.5, or 7 days, for example, at least 2 days and no more than 7 days, and measuring CAR expression level in the portion (for example, measuring the percentage of viable, CAR-expressing cells in the portion).

In some embodiments, provided herein is a method of making a population of cells (for example, T cells) that comprise: a first nucleic acid molecule that encodes a controllable chimeric antigen receptor (CCAR), or a second nucleic acid molecule that encodes a chimeric antigen receptor (CAR) and a regulatory molecule. In some embodiments, this disclosure features a method of making a population of cells (for example, T cells) that comprise a first nucleic acid molecule that encodes a controllable chimeric antigen receptor (CCAR). In some embodiments, this disclosure features a method of making a population of cells (for example, T cells) that comprise a second nucleic acid molecule that encodes a chimeric antigen receptor (CAR) and a regulatory molecule. In some embodiments, the second nucleic acid molecule comprises one or more nucleic acid molecules, e.g., the second nucleic acid molecule comprises a third nucleic acid molecule and a fourth nucleic acid molecule, wherein the third nucleic acid molecule comprises a nucleic acid sequence encoding the CAR and the fourth nucleic acid molecule comprises a nucleic acid sequence encoding the regulatory molecule.

In some embodiments, the method comprises: (1) contacting a population of cells (for example, T cells, for example, T cells isolated from a frozen leukapheresis product) with a cytokine chosen from IL-2, IL-7, IL-15, IL-21, IL-6, or a combination thereof, (2) contacting the population of cells (for example, T cells) with a first nucleic acid molecule (for example, a DNA or RNA molecule) encoding a CCAR or a second nucleic acid molecule (for example, a DNA or RNA molecule) encoding a CAR and a regulatory molecule, thereby providing a population of cells (for example, T cells) comprising the first or second nucleic acid molecule, and (3) harvesting the population of cells (for example, T cells) for storage (for example, reformulating the population of cells in cryopreservation media) or administration. In some embodiments, step (2) is performed together with step (1) or no later than 5 hours after the beginning of step (1), for example, no later than 1, 2, 3, 4, or 5 hours after the beginning of step (1), and step (3) is performed no later than 26 hours after the beginning of step (1), for example, no later than 22, 23, or 24 hours after the beginning of step (1), for example, no later than 24 hours after the beginning of step (1). In some embodiments, the population of cells from step (3) are not expanded, or expanded by no more than 5, 10, 15, 20, 25, 30, 35, or 40%, for example, no more than 10%, for example, as assessed by the number of living cells, compared to the population of cells at the beginning of step (1). In some embodiments, the first or second nucleic acid molecule in step (2) is on a viral vector. In some embodiments, the first or second nucleic acid molecule in step (ii) is an RNA molecule on a viral vector. In some embodiments, step (ii) comprises transducing the population of cells (for example, T cells) with a viral vector comprising the first or second nucleic acid molecule.

In some embodiments, step (1) comprises contacting the population of cells (for example, T cells) with IL-2. In some embodiments, step (1) comprises contacting the population of cells (for example, T cells) with IL-7. In some embodiments, step (1) comprises contacting the population of cells (for example, T cells) with IL-15 (for example, hetIL-15 (IL15/sIL-15Ra)). In some embodiments, step (1) comprises contacting the population of cells (for example, T cells) with IL-21. In some embodiments, step (1) comprises contacting the population of cells (for example, T cells) with IL-6 (for example, IL-6/sIL-6Ra). In some embodiments, step (1) comprises contacting the population of cells (for example, T cells) with IL-7 and IL-15 (for example, hetIL-15 (IL15/sIL-15Ra)). In some embodiments, step (1) comprises contacting the population of cells (for example, T cells) with IL-7 and IL-21. In some embodiments, step (1) comprises contacting the population of cells (for example, T cells) with IL-15 (for example, hetIL-15 (IL15/sIL-15Ra)) and IL-21. In some embodiments, step (1) comprises contacting the population of cells (for example, T cells) with IL-7, IL-15 (for example, hetIL-15 (IL15/sIL-15Ra)), and IL-21. In some embodiments, step (1) comprises contacting the population of cells (for example, T cells) with IL-6 (for example, IL-6/sIL-6Ra) and IL-15 (for example, hetIL-15 (IL15/sIL-15Ra)). In some embodiments, step (1) comprises contacting the population of cells (for example, T cells) with IL-2 and IL-6 (for example, IL-6/sIL-6Ra).

In some embodiments, the population of cells from step (3) shows a higher percentage of naïve cells among cells that comprise the first or second nucleic acid molecule (for example, at least 10, 15, 20, 25, 30, 35, or 40% higher), compared with cells made by an otherwise similar method which further comprises contacting the population of cells with, for example, an anti-CD3 antibody.

In some embodiments, the percentage of naïve cells, for example, naïve T cells, for example, CD45RA+ CD45RO− CCR7+ T cells, in the population of cells from step (3): (a) is the same as or differs by no more than 5 or 10% from the percentage of naïve cells, for example, naïve T cells, for example, CD45RA+ CD45RO− CCR7+ cells, in the population of cells at the beginning of step (1), or (b) is increased, for example, increased by at least 10 or 20%, as compared to the percentage of naïve cells, for example, naïve T cells, for example, CD45RA+ CD45RO− CCR7+ cells, in the population of cells at the beginning of step (1).

In some embodiments, the population of cells from step (3) shows a higher percentage of naïve cells, for example, naïve T cells, for example, CD45RA+ CD45RO− CCR7+ T cells (for example, at least 10, 20, 30, or 40% higher), compared with cells made by an otherwise similar method in which step (3) is performed more than 26 hours after the beginning of step (1), for example, more than 5, 6, 7, 8, 9, 10, 11, or 12 days after the beginning of step (1).

In some embodiments, the population of cells from step (3) shows a higher percentage of naïve cells, for example, naïve T cells, for example, CD45RA+ CD45RO− CCR7+ T cells (for example, at least 10, 20, 30, or 40% higher), compared with cells made by an otherwise similar method which further comprises, after step (2) and prior to step (3), expanding the population of cells (for example, T cells) in vitro for more than 3 days, for example, for 5, 6, 7, 8 or 9 days.

In some embodiments, the population of cells from step (3), after being administered in vivo, persists longer or expands at a higher level (for example, as assessed using methods described in Example 1 with respect to FIG. 4C), compared with cells made by an otherwise similar method in which step (3) is performed more than 26 hours after the beginning of step (1), for example, more than 5, 6, 7, 8, 9, 10, 11, or 12 days after the beginning of step (1).

In some embodiments, the population of cells from step (3), after being administered in vivo, persists longer or expands at a higher level (for example, as assessed using methods described in Example 1 with respect to FIG. 4C), compared with cells made by an otherwise similar method which further comprises, after step (2) and prior to step (3), expanding the population of cells (for example, T cells) in vitro for more than 3 days, for example, for 5, 6, 7, 8 or 9 days.

In some embodiments, the population of cells from step (3) are not expanded, or expanded by no more than 5, 10, 15, 20, 25, 30, 35, or 40%, for example, no more than 10%, for example, as assessed by the number of living cells, compared to the population of cells at the beginning of step (1), optionally wherein the number of living cells in the population of cells from step (3) decreases from the number of living cells in the population of cells at the beginning of step (1).

In some embodiments, the population of cells from step (3) are not expanded, or expanded by less than 2 hours, for example, less than 1 or 1.5 hours, compared to the population of cells at the beginning of step (1).

In some embodiments, the population of cells is not contacted in vitro with an agent that stimulates a CD3/TCR complex and/or an agent that stimulates a costimulatory molecule on the surface of the cells, or if contacted, the contacting step is less than 2 hours, for example, no more than 1 or 1.5 hours. In some embodiments, the agent that stimulates a CD3/TCR complex is an agent that stimulates CD3 (for example, an anti-CD3 antibody) and the agent that stimulates a costimulatory molecule is an agent that stimulates CD28, ICOS, CD27, HVEM, LIGHT, CD40, 4-1BB, OX40, DR3, GITR, CD30, TIM1, CD2, CD226, or any combination thereof, optionally wherein the agent that stimulates a CD3/TCR complex or the agent that stimulates a costimulatory molecule is chosen from an antibody (for example, a single-domain antibody (for example, a heavy chain variable domain antibody), a peptibody, a Fab fragment, or a scFv), a small molecule, or a ligand (for example, a naturally-existing, recombinant, or chimeric ligand).

In some embodiments, steps (1) and/or (2) are performed in cell media comprising: no more than 5, 4, 3, 2, 1, or 0% serum, optionally wherein steps (1) and/or (2) are performed in cell media comprising about 2% serum, or a LSD1 inhibitor or a MALT1 inhibitor.

In some embodiments, the method further comprises receiving a cryopreserved leukapheresis product (or an alternative source of hematopoietic tissue such as a cryopreserved whole blood product, a cryopreserved bone marrow product, or a cryopreserved tumor or organ biopsy or removal (for example, a cryopreserved product from thymectomy)) from an entity, for example, a laboratory, hospital, or healthcare provider.

In some embodiments, the population of cells at the beginning of step (i) or step (1) has been enriched for IL6R-expressing cells (for example, cells that are positive for IL6Rα and/or IL6Rβ). In some embodiments, the population of cells at the beginning of step (i) or step (1) comprises no less than 50, 60, or 70% of IL6R-expressing cells (for example, cells that are positive for IL6Rα and/or IL6Rβ). In some embodiments, steps (i) and (ii) or steps (1) and (2) are performed in cell media comprising IL-15 (for example, hetIL-15 (IL15/sIL-15Ra)). In some embodiments, IL-15 increases the ability of the population of cells to expand, for example, 10, 15, 20, or 25 days later. In some embodiments, IL-15 increases the percentage of IL6Rβ-expressing cells in the population of cells.

In some embodiments, the CCAR or CAR comprises an antigen binding domain, a transmembrane domain, and/or an intracellular signaling domain. In some embodiments, the antigen binding domain binds to an antigen chosen from: CD19, CD20, CD22, BCMA, mesothelin, EGFRvIII, GD2, Tn antigen, sTn antigen, Tn-O-Glycopeptides, sTn-O-Glycopeptides, PSMA, CD97, TAG72, CD44v6, CEA, EPCAM, KIT, IL-13Ra2, leguman, GD3, CD171, IL-11Ra, PSCA, MAD-CT-1, MAD-CT-2, VEGFR2, LewisY, CD24, PDGFR-beta, SSEA-4, folate receptor alpha, ERBBs (for example, ERBB2), Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, NY-ESO-1, GPR20, Ly6k, OR51E2, TARP, GFRα4, or a peptide of any of these antigens presented on MHC. In some embodiments, the antigen binding domain comprises a CDR, VH, VL, or scFv sequence disclosed herein, optionally wherein: (a) the antigen binding domain binds to BCMA and comprises a CDR, VH, VL, scFv or CAR sequence disclosed in Tables 3-15, or a sequence having at least 80%, 85%, 90%, 95%, or 99% identity thereto; (b) the antigen binding domain binds to CD19 and comprises a CDR, VH, VL, scFv or CAR sequence disclosed in Table 2, or a sequence having at least 80%, 85%, 90%, 95%, or 99% identity thereto; (c) the antigen binding domain binds to CD20 and comprises a CDR, VH, VL, scFv or CAR sequence disclosed herein, or a sequence having at least 80%, 85%, 90%, 95%, or 99% identity thereto; or (d) the antigen binding domain binds to CD22 and comprises a CDR, VH, VL, scFv or CAR sequence disclosed herein, or a sequence having at least 80%, 85%, 90%, 95%, or 99% identity thereto. In some embodiments, the antigen binding domain comprises a VH and a VL, wherein the VH and VL are connected by a linker, optionally wherein the linker comprises the amino acid sequence of SEQ ID NO: 63 or 104. In some embodiments, (a) the transmembrane domain comprises a transmembrane domain of a protein chosen from the alpha, beta or zeta chain of T-cell receptor, CD28, CD3 epsilon, CD45, CD4, CD5, CD8, CD9, CD16, CD22, CD33, CD37, CD64, CD80, CD86, CD134, CD137 and CD154, (b) the transmembrane domain comprises a transmembrane domain of CD8, (c) the transmembrane domain comprises the amino acid sequence of SEQ ID NO: 6, or an amino acid sequence having at least about 85%, 90%, 95%, or 99% sequence identity thereof, or (d) the first or second nucleic acid molecule comprises a nucleic acid sequence encoding the transmembrane domain, wherein the nucleic acid sequence comprises the nucleic acid sequence of SEQ ID NO: 17, or a nucleic acid sequence having at least about 85%, 90%, 95%, or 99% sequence identity thereof. In some embodiments, the antigen binding domain is connected to the transmembrane domain by a hinge region, optionally wherein: (a) the hinge region comprises the amino acid sequence of SEQ ID NO: 2, 3, or 4, or an amino acid sequence having at least about 85%, 90%, 95%, or 99% sequence identity thereof, or (b) the first or second nucleic acid molecule comprises a nucleic acid sequence encoding the hinge region, wherein the nucleic acid sequence comprises the nucleic acid sequence of SEQ ID NO: 13, 14, or 15, or a nucleic acid sequence having at least about 85%, 90%, 95%, or 99% sequence identity thereof. In some embodiments, the intracellular signaling domain comprises a primary signaling domain, optionally wherein the primary signaling domain comprises a functional signaling domain derived from CD3 zeta, TCR zeta, FcR gamma, FcR beta, CD3 gamma, CD3 delta, CD3 epsilon, CD5, CD22, CD79a, CD79b, CD278 (ICOS), FcεRI, DAP10, DAP12, or CD66d, optionally wherein: (a) the primary signaling domain comprises a functional signaling domain derived from CD3 zeta, (b) the primary signaling domain comprises the amino acid sequence of SEQ ID NO: 9 or 10, or an amino acid sequence having at least about 85%, 90%, 95%, or 99% sequence identity thereof, or (c) the first or second nucleic acid molecule comprises a nucleic acid sequence encoding the primary signaling domain, wherein the nucleic acid sequence comprises the nucleic acid sequence of SEQ ID NO: 20 or 21, or a nucleic acid sequence having at least about 85%, 90%, 95%, or 99% sequence identity thereof. In some embodiments, the intracellular signaling domain comprises a costimulatory signaling domain, optionally wherein the costimulatory signaling domain comprises a functional signaling domain derived from a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CD5, ICAM-1, 4-1BB (CD137), B7-H3, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, NKG2D, 20 NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD28-OX40, CD28-4-1BB, or a ligand that specifically binds with CD83. In some embodiments, (a) the costimulatory signaling domain comprises a functional signaling domain derived from 4-1BB, (b) the costimulatory signaling domain comprises the amino acid sequence of SEQ ID NO: 7, or an amino acid sequence having at least about 85%, 90%, 95%, or 99% sequence identity thereof, or (c) the first or second nucleic acid molecule comprises a nucleic acid sequence encoding the costimulatory signaling domain, wherein the nucleic acid sequence comprises the nucleic acid sequence of SEQ ID NO: 18, or a nucleic acid sequence having at least about 85%, 90%, 95%, or 99% sequence identity thereof. In some embodiments, the intracellular signaling domain comprises a functional signaling domain derived from 4-1BB and a functional signaling domain derived from CD3 zeta, optionally wherein the intracellular signaling domain comprises the amino acid sequence of SEQ ID NO: 7 (or an amino acid sequence having at least about 85%, 90%, 95%, or 99% sequence identity thereof) and the amino acid sequence of SEQ ID NO: 9 or 10 (or an amino acid sequence having at least about 85%, 90%, 95%, or 99% sequence identity thereof), optionally wherein the intracellular signaling domain comprises the amino acid sequence of SEQ ID NO: 7 and the amino acid sequence of SEQ ID NO: 9 or 10. In some embodiments, the CCAR or CAR further comprises a leader sequence comprising the amino acid sequence of SEQ ID NO: 1.

In some embodiments, provided herein is a population of cells that comprise the first or second nucleic acid molecule (for example, autologous or allogeneic T cells or NK cells that comprise the first or second nucleic acid molecule) made by the aforementioned methods.

In some embodiments, provided herein is a population of cells engineered to comprise: a first nucleic acid molecule that encodes a CCAR, or a second nucleic acid molecule that encodes a CAR and a regulatory molecule. In some embodiments, provided herein is a population of cells engineered to comprise a first nucleic acid molecule that encodes a CCAR. In some embodiments, provided herein is a population of cells engineered to comprise a second nucleic acid molecule that encodes a CAR and a regulatory molecule. In some embodiments, the second nucleic acid molecule comprises one or more nucleic acid molecules, e.g., the second nucleic acid molecule comprises a third nucleic acid molecule and a fourth nucleic acid molecule, wherein the third nucleic acid molecule comprises a nucleic acid sequence encoding the CAR and the fourth nucleic acid molecule comprises a nucleic acid sequence encoding the regulatory molecule.

In some embodiments, the population comprises: (a) about the same percentage of naïve cells, for example, naïve T cells, for example, CD45RO− CCR7+ T cells, as compared to the percentage of naïve cells, for example, naïve T cells, for example, CD45RO− CCR7+ cells, in the same population of cells prior to being engineered to comprise the first or second nucleic acid molecule; (b) a change within about 5% to about 10% of naïve cells, for example, naïve T cells, for example, CD45RO− CCR7+ T cells, for example, as compared to the percentage of naïve cells, for example, naïve T cells, for example, CD45RO− CCR7+ cells, in the same population of cells prior to being engineered to comprise the first or second nucleic acid molecule; (c) an increased percentage of naïve cells, for example, naïve T cells, for example, CD45RO− CCR7+ T cells, for example, increased by at least 1.2, 1.4, 1.6, 1.8, 2.0, 2.2, 2.4, 2.6, 2.8, or 3-fold, as compared to the percentage of naïve cells, for example, naïve T cells, for example, CD45RO− CCR7+ cells, in the same population of cells prior to being engineered to comprise the first or second nucleic acid molecule; (d) about the same percentage of central memory cells, for example, central memory T cells, for example, CCR7+CD45RO+ T cells, as compared to the percentage of central memory cells, for example, central memory T cells, for example, CCR7+CD45RO+ T cells, in the same population of cells prior to being engineered to comprise the first or second nucleic acid molecule; (e) a change within about 5% to about 10% of central memory cells, for example, central memory T cells, for example, CCR7+CD45RO+ T cells, as compared to the percentage of central memory cells, for example, central memory T cells, for example, CCR7+CD45RO+ T cells, in the same population of cells prior to being engineered to comprise the first or second nucleic acid molecule; (f) a decreased percentage of central memory cells, for example, central memory T cells, for example, CCR7+CD45RO+ T cells, for example, decreased by at least 20, 25, 30, 35, 40, 45, or 50%, as compared to the percentage of central memory cells, for example, central memory T cells, for example, CCR7+CD45RO+ T cells, in the same population of cells prior to being engineered to comprise the first or second nucleic acid molecule; (g) about the same percentage of stem memory T cells, for example, CD45RA+CD95+IL-2 receptor β+CCR7+CD62L+ T cells, as compared to the percentage of stem memory T cells, for example, CD45RA+CD95+IL-2 receptor β+CCR7+CD62L+ T cells, in the same population of cells prior to being engineered to comprise the first or second nucleic acid molecule; (h) a change within about 5% to about 10% of stem memory T cells, for example, CD45RA+CD95+IL-2 receptor β+CCR7+CD62L+ T cells, as compared to the percentage of stem memory T cells, for example, CD45RA+CD95+IL-2 receptor β+CCR7+CD62L+ T cells, in the same population of cells prior to being engineered to comprise the first or second nucleic acid molecule; or (i) an increased percentage of stem memory T cells, for example, CD45RA+CD95+IL-2 receptor β+CCR7+CD62L+ T cells, as compared to the percentage of stem memory T cells, for example, CD45RA+CD95+IL-2 receptor β+CCR7+CD62L+ T cells, in the same population of cells prior to being engineered to comprise the first or second nucleic acid molecule.

In some embodiments, provided herein is a population of cells engineered to comprise: a first nucleic acid molecule that encodes a CCAR, or a second nucleic acid molecule that encodes a CAR and a regulatory molecule, wherein: (a) the median GeneSetScore (Up TEM vs. Down TSCM) of the population of cells is about the same as or differs by no more than (for example, increased by no more than) about 25, 50, 75, 100, or 125% from the median GeneSetScore (Up TEM vs. Down TSCM) of the same population of cells prior to being engineered to comprise the first or second nucleic acid molecule; (b) the median GeneSetScore (Up Treg vs. Down Teff) of the population of cells is about the same as or differs by no more than (for example, increased by no more than) about 25, 50, 100, 150, or 200% from the median GeneSetScore (Up Treg vs. Down Teff) of the population of cells prior to being engineered to comprise the first or second nucleic acid molecule; (c) the median GeneSetScore (Down stemness) of the population of cells is about the same as or differs by no more than (for example, increased by no more than) about 25, 50, 100, 150, 200, or 250% from the median GeneSetScore (Down stemness) of the population of cells prior to being engineered to comprise the first or second nucleic acid molecule; (d) the median GeneSetScore (Up hypoxia) of the population of cells is about the same as or differs by no more than (for example, increased by no more than) about 125, 150, 175, or 200% from the median GeneSetScore (Up hypoxia) of the population of cells prior to being engineered to comprise the first or second nucleic acid molecule; or (e) the median GeneSetScore (Up autophagy) of the population of cells is about the same as or differs by no more than (for example, increased by no more than) about 180, 190, 200, or 210% from the median GeneSetScore (Up autophagy) of the population of cells prior to being engineered to comprise the first or second nucleic acid molecule.

In some embodiments, the population of cells comprise the first nucleic acid molecule that encodes a CCAR.

In some embodiments, the CCAR is a fusion polypeptide comprising a degradation polypeptide (e.g., a degradation polypeptide disclosed herein) and a CAR polypeptide (e.g., a CAR polypeptide disclosed herein). In some embodiments, (i) the degradation polypeptide comprises or consists of an amino acid sequence selected from the group consisting of SEQ ID NOs: 310-315, 320-324, 337-339, 360-361, 367-369 and 374 (or a sequence having at least 85, 87, 90, 95, 97, 98, 99, or 100% identity thereto), optionally wherein the degradation polypeptide comprises or consists of the amino acid sequence of SEQ ID NO: 312; (ii) the degradation polypeptide comprises a beta turn of IKZF1 or IKZF3 (or a sequence having at least 85, 87, 90, 95, 97, 98, 99, or 100% identity thereto), optionally wherein the degradation polypeptide comprises a beta hairpin or a beta strand of IKZF1 or IKZF3 (or a sequence having at least 85, 87, 90, 95, 97, 98, 99, or 100% identity thereto); (iii) the degradation polypeptide comprises an alpha helix of IKZF1 or IKZF3 (or a sequence having at least 85, 87, 90, 95, 97, 98, 99, or 100% identity thereto); (iv) the degradation polypeptide comprises, from the N-terminus to the C-terminus, a first beta strand, a beta hairpin, a second beta strand, and a first alpha helix of IKZF1 or IKZF3 (or a sequence having at least 85, 87, 90, 95, 97, 98, 99, or 100% identity thereto); (v) the degradation polypeptide comprises, from the N-terminus to the C-terminus, a first beta strand, a beta hairpin, a second beta strand, a first alpha helix, and a second alpha helix of IKZF1 or IKZF3 (or a sequence having at least 85, 87, 90, 95, 97, 98, 99, or 100% identity thereto), optionally wherein the beta hairpin and the second alpha helix are separated by no more than 60, 50, 40, or 30 amino acid residues; (vi) the degradation polypeptide comprises about 10 to about 95 amino acid residues, about 15 to about 90 amino acid residues, about 20 to about 85 amino acid residues, about 25 to about 80 amino acid residues, about 30 to about 75 amino acid residues, about 35 to about 70 amino acid residues, about 40 to about 65 amino acid residues, about 45 to about 65 amino acid residues, about 50 to about 65 amino acid residues, or about 55 to about 65 amino acid residues of IKZF1 or IKZF3 (or a sequence having at least 85, 87, 90, 95, 97, 98, 99, or 100% identity thereto); (vii) the degradation polypeptide comprises at least 10 amino acids, at least 15 amino acids, at least 20 amino acids, at least 25 amino acids, at least 30 amino acids, at least 35 amino acids, at least 40 amino acids, at least 45 amino acids, at least 50 amino acids, at least 55 amino acids, at least 60 amino acids, at least 65 amino acids, at least 70 amino acids, at least 75 amino acids, at least 80 amino acids, at least 85 amino acids, at least 90 amino acids, at least 90 amino acids, or at least 95 amino acids of IKZF1 or IKZF3 (or a sequence having at least 85, 87, 90, 95, 97, 98, 99, or 100% identity thereto); (viii) the association of the fusion polypeptide with cereblon (CRBN) in the absence of COF1 or COF2, e.g., an immunomodulatory imide drug (IMiD), e.g., lenalidomide, pomalidomide, or thalidomide, is no more than, e.g., 0.01%, 0.1%, 1%, 5%, 10%, 15%, or 20%, of the association of the fusion polypeptide with CRBN in the presence of COF1 or COF2, e.g., an IMiD, e.g., lenalidomide, pomalidomide, or thalidomide; (ix) the ubiquitination of the fusion polypeptide in the absence of COF1 or COF2, e.g., an IMiD, e.g., lenalidomide, pomalidomide, or thalidomide, is no more than, e g , 0.01%, 0.1%, 1%, 10%, 20%, 30%, 40%, 50%, 60%, or 70%, of the ubiquitination of the fusion polypeptide in the presence of COF1 or COF2, e.g., an IMiD, e.g., lenalidomide, pomalidomide, or thalidomide; (x) the degradation of the fusion polypeptide in the absence of COF1 or COF2, e.g., an IMiD, e.g., lenalidomide, pomalidomide, or thalidomide, is no more than, e.g., 0.01%, 0.1%, 1%, 10%, 20%, 30%, 40%, 50%, 60%, or 70% of the degradation of the fusion polypeptide in the presence of COF1 or COF2, e.g., an IMiD, e.g., lenalidomide, pomalidomide, or thalidomide; and/or (xi) the expression level of the fusion polypeptide in the presence of COF1 or COF2, e.g., an IMiD, e.g., lenalidomide, pomalidomide, or thalidomide, is decreased by, e.g., at least 40, 50, 60, 70, 80, 90, or 99%, as compared to the expression level of the fusion polypeptide in the absence of COF1 or COF2, e.g., an IMiD, e.g., lenalidomide, pomalidomide, or thalidomide.

In some embodiments, the degradation polypeptide comprises or consists of an amino acid sequence selected from the group consisting of SEQ ID NOs: 375-377 (or a sequence having at least 85, 87, 90, 95, 97, 98, 99, or 100% identity thereto), optionally wherein the degradation polypeptide comprises or consists of the amino acid sequence of SEQ ID NO: 375. In some embodiments, the degradation polypeptide comprises a beta turn of IKZF2 (or a sequence having at least 85, 87, 90, 95, 97, 98, 99, or 100% identity thereto), optionally wherein the degradation polypeptide comprises a beta hairpin or a beta strand of IKZF2 (or a sequence having at least 85, 87, 90, 95, 97, 98, 99, or 100% identity thereto). In some embodiments, the degradation polypeptide comprises an alpha helix of IKZF2 (or a sequence having at least 85, 87, 90, 95, 97, 98, 99, or 100% identity thereto). In some embodiments, the degradation polypeptide comprises, from the N-terminus to the C-terminus, a first beta strand, a beta hairpin, a second beta strand, and a first alpha helix of IKZF2 (or a sequence having at least 85, 87, 90, 95, 97, 98, 99, or 100% identity thereto). In some embodiments, the degradation polypeptide comprises, from the N-terminus to the C-terminus, a first beta strand, a beta hairpin, a second beta strand, a first alpha helix, and a second alpha helix of IKZF2 (or a sequence having at least 85, 87, 90, 95, 97, 98, 99, or 100% identity thereto), optionally wherein the beta hairpin and the second alpha helix are separated by no more than 60, 50, 40, or 30 amino acid residues. In some embodiments, the degradation polypeptide comprises about 10 to about 95 amino acid residues, about 15 to about 90 amino acid residues, about 20 to about 85 amino acid residues, about 25 to about 80 amino acid residues, about 30 to about 75 amino acid residues, about 35 to about 70 amino acid residues, about 40 to about 65 amino acid residues, about 45 to about 65 amino acid residues, about 50 to about 65 amino acid residues, or about 55 to about 65 amino acid residues of IKZF2 (or a sequence having at least 85, 87, 90, 95, 97, 98, 99, or 100% identity thereto). In some embodiments, the degradation polypeptide comprises at least 10 amino acids, at least 15 amino acids, at least 20 amino acids, at least 25 amino acids, at least 30 amino acids, at least 35 amino acids, at least 40 amino acids, at least 45 amino acids, at least 50 amino acids, at least 55 amino acids, at least 60 amino acids, at least 65 amino acids, at least 70 amino acids, at least 75 amino acids, at least 80 amino acids, at least 85 amino acids, at least 90 amino acids, at least 90 amino acids, or at least 95 amino acids of IKZF2 (or a sequence having at least 85, 87, 90, 95, 97, 98, 99, or 100% identity thereto). In some embodiments, the association of the fusion polypeptide with cereblon (CRBN) in the absence of COF3, e.g., Compound I-112 disclosed in Table 29, is no more than, e.g., 0.01%, 0.1%, 1%, 5%, 10%, 15%, or 20%, of the association of the fusion polypeptide with CRBN in the presence of COF3, e.g., Compound I-112 disclosed in Table 29. In some embodiments, the ubiquitination of the fusion polypeptide in the absence of COF3, e.g., Compound I-112 disclosed in Table 29, is no more than, e.g., 0.01%, 0.1%, 1%, 10%, 20%, 30%, 40%, 50%, 60%, or 70%, of the ubiquitination of the fusion polypeptide in the presence of COF3, e.g., Compound I-112 disclosed in Table 29. In some embodiments, the degradation of the fusion polypeptide in the absence of COF3, e.g., Compound I-112 disclosed in Table 29, is no more than, e.g., 0.01%, 0.1%, 1%, 10%, 20%, 30%, 40%, 50%, 60%, or 70% of the degradation of the fusion polypeptide in the presence of COF3, e.g., Compound I-112 disclosed in Table 29. In some embodiments, the expression level of the fusion polypeptide in the presence of COF3, e.g., Compound I-112 disclosed in Table 29, is decreased by, e.g., at least 40, 50, 60, 70, 80, 90, or 99%, as compared to the expression level of the fusion polypeptide in the absence of COF3, e.g., Compound I-112 disclosed in Table 29.

In some embodiments, (i) the degradation polypeptide is fused to the CAR polypeptide; (ii) the degradation polypeptide and the CAR polypeptide are linked by a peptide bond; (iii) the degradation polypeptide and the CAR polypeptide are linked by a bond other than a peptide bond; (iv) the degradation polypeptide is linked directly to the CAR polypeptide; (v) the degradation polypeptide is linked indirectly to the CAR polypeptide; (vi) the degradation polypeptide and the CAR polypeptide are operatively linked via a linker, e.g., a glycine-serine linker, e.g., a linker comprising the amino acid sequence of GGGGSGGGGTGGGGSG (SEQ ID NO: 335); (vii) the degradation polypeptide is linked to the C-terminus or N-terminus of the CAR polypeptide; or (viii) the degradation polypeptide is at the middle of the CAR polypeptide.

In some embodiments, the CCAR is a fusion polypeptide comprising a degradation domain (e.g., a degradation domain disclosed herein) and a CAR polypeptide (e.g., a CAR polypeptide disclosed herein), optionally wherein the degradation domain is separated from the CAR polypeptide by a heterologous protease cleavage site, optionally wherein the CCAR comprises, from the N-terminus to the C-terminus, the degradation domain, the heterologous protease cleavage site, and the CAR polypeptide.

In some embodiments, the degradation domain has a first state associated with a first level of expression of the fusion polypeptide and a second state associated with a second level of expression of the fusion polypeptide, wherein the second level is increased, e.g., by at least 2-, 3-, 4-, 5-, 10-, 20- or 30-fold over the first level in the presence of a stabilization compound, optionally wherein: (a) in the absence of the stabilization compound, the fusion polypeptide is degraded by a cellular degradation pathway, e.g., at least 50%, 60%, 70%, 80%, 90% or greater of the fusion polypeptide is degraded; (b) in the presence of the stabilization compound, the degradation domain assumes a conformation more resistant to cellular degradation relative to a conformation in the absence of the stabilization compound; and/or (c) in the presence of the stabilization compound, the conformation of the fusion polypeptide is more permissive to cleavage at the heterologous protease cleavage site relative to a conformation in the absence of the stabilization compound.

In some embodiments, the degradation domain is chosen from an estrogen receptor (ER) domain, an FKB protein (FKBP) domain, or a dihydrofolate reductase (DHFR) domain, optionally wherein: (a) the degradation domain is an estrogen receptor (ER) domain, e.g., the degradation domain comprising the amino acid sequence of SEQ ID NO: 342 or 344, or a sequence having at least 85, 87, 90, 95, 97, 98, 99, or 100% identity thereto, optionally wherein the stabilization compound is bazedoxifene or 4-hydroxy tamoxifen (4-OHT), or a pharmaceutically acceptable salt thereof; (b) the degradation domain is an FKB protein (FKBP) domain, e.g., the degradation domain comprising the amino acid sequence of SEQ ID NO: 346, or a sequence having at least 85, 87, 90, 95, 97, 98, 99, or 100% identity thereto, optionally wherein the stabilization compound is Shield-1, or a pharmaceutically acceptable salt thereof; or (c) the degradation domain is a dihydrofolate reductase (DHFR) domain, e.g., the degradation domain comprising the amino acid sequence of SEQ ID NO: 347, or a sequence having at least 85, 87, 90, 95, 97, 98, 99, or 100% identity thereto, optionally wherein the stabilization compound is trimethoprim, or a pharmaceutically acceptable salt thereof.

In some embodiments, the heterologous protease cleavage site is cleaved by a mammalian intracellular protease, optionally wherein: (a) the heterologous protease cleavage site is cleaved by a protease selected from the group consisting of furin, PCSK1, PCSK5, PCSK6, PCSK7, cathepsin B, Granzyme B, Factor XA, Enterokinase, genenase, sortase, precission protease, thrombin, TEV protease, and elastase 1; (b) the heterologous protease cleavage site comprises a sequence having a cleavage motif selected from the group consisting of RX(K/R)R consensus motif (X can be any amino acid; SEQ ID NO: 348), RXXX[KR]R consensus motif (X can be any amino acid; SEQ ID NO: 349), RRX consensus motif (SEQ ID NO : 350), I-E-P-D-X consensus motif (SEQ ID NO: 351), Ile-Glu/Asp-Gly-Arg (SEQ ID NO: 352), Asp-Asp-Asp-Asp-Lys (SEQ ID NO: 353), Pro-Gly-Ala-Ala-His-Tyr (SEQ ID NO: 354), LPXTG/A consensus motif (SEQ ID NO: 355), Leu-Glu-Val-Phe-Gln-Gly-Pro (SEQ ID NO: 356), Leu-Val-Pro-Arg-Gly-Ser (SEQ ID NO: 357), E-N-L-Y-F-Q-G (SEQ ID NO: 358), and [AGSV]-X (X can be any amino acid; SEQ ID NO: 359); or (c) the heterologous protease cleavage site comprises a furin cleavage site selected from the group consisting of RTKR (SEQ ID NO: 378); GTGAEDPRPSRKRRSLGDVG (SEQ ID NO: 379); GTGAEDPRPSRKRR (SEQ ID NO: 381); LQWLEQQVAKRRTKR (SEQ ID NO: 383); GTGAEDPRPSRKRRSLGG (SEQ ID NO: 385); GTGAEDPRPSRKRRSLG (SEQ ID NO: 387); SLNLTESHNSRKKR (SEQ ID NO: 389); CKINGYPKRGRKRR (SEQ ID NO: 391); and SARNRQKR (SEQ ID NO: 336). In some embodiments, the heterologous protease cleavage site is cleaved by a mammalian extracellular protease, optionally wherein: (a) the heterologous protease cleavage site is cleaved by a protease selected from the group consisting of Factor XA, Enterokinase, genenase, sortase, precission protease, thrombin, TEV protease, and elastase 1; or (b) the heterologous protease cleavage site comprises an amino acid sequence selected from the group consisting of Ile-Glu/Asp-Gly-Arg (SEQ IDNO : 352), Asp-Asp-Asp-Asp-Lys (SEQ ID NO: 353), Pro-Gly-Ala-Ala-His-Tyr (SEQ ID NO: 354), LPXTG/A consensus motif (SEQ ID NO: 355), Leu-Glu-Val-Phe-Gln-Gly-Pro (SEQ ID NO: 356), Leu-Val-Pro-Arg-Gly-Ser (SEQ ID NO: 357), E-N-L-Y-F-Q-G (SEQ ID NO: 358), and [AGSV]-X (X can be any amino acid; SEQ ID NO: 359).

In some embodiments, the CCAR is a regulatable CAR (RCAR) (e.g., an RCAR disclosed herein). In some embodiments, the RCAR comprises: (i) an intracellular signaling member comprising: an intracellular signaling domain, e.g., a primary intracellular signaling domain, and a first switch domain; (ii) an antigen binding member comprising: an antigen binding domain and a second switch domain; and (iii) a transmembrane domain, optionally wherein the transmembrane domain can be disposed on the intracellular signaling member and/or the antigen binding member. In some embodiments, the RCAR comprises: (i) an intracellular signaling member comprising: an intracellular signaling domain, e.g., a primary intracellular signaling domain, and a first switch domain; (ii) an inhibitory extracellular domain member comprising: an inhibitory extracellular domain (e.g., an inhibitory extracellular domain comprising an extracellular domain of B7-H1, B7-1, CD160, P1H, 2B4, PD1, TIM3, CEACAM, LAG3, TIGIT, CTLA-4, BTLA, LAIR1, or TGF-beta receptor, or a sequence having at least 85, 87, 90, 95, 97, 98, 99, or 100% identity thereto), and a second switch domain; and (iii) a transmembrane domain, optionally wherein the transmembrane domain can be disposed on the intracellular signaling member and/or the inhibitory extracellular domain member. In some embodiments, the RCAR comprises: (i) an intracellular signaling member comprising: an intracellular signaling domain, e.g., a primary intracellular signaling domain, and a first switch domain; (ii) a costimulatory extracellular domain member comprising: a costimulatory extracellular domain (e.g., a costimulatory extracellular domain comprising an extracellular domain of ICOS, CD28, VEM, LIGHT, CD40L, 4-1BB, OX40, DR3, GITR, CD30, TIM1, SLAM, CD2, or CD226, or a sequence having at least 85, 87, 90, 95, 97, 98, 99, or 100% identity thereto), and a second switch domain; and (iii) a transmembrane domain, optionally wherein the transmembrane domain can be disposed on the intracellular signaling member and/or the costimulatory extracellular domain member.

In some embodiments, the first and second switch domains can form a dimerization switch, e.g., in the presence of a dimerization molecule, optionally wherein: (i) the dimerization switch is an intracellular dimerization switch or an extracellular dimerization switch; (ii) the dimerization switch is a homodimerization switch or a heterodimerization switch; (iii) the dimerization switch comprises a FKBP-FRB based switch, e.g., a dimerization switch comprising a switch domain comprising a FRB binding fragment or analog of FKBP and a switch domain comprising a FKBP binding fragment or analog of FRB, optionally wherein the FKBP binding fragment or analog of FRB comprises one or more mutations disclosed herein (e.g., one or more mutations chosen from an E2032 mutation, a T2098 mutation, or an E2032 and a T2098 mutation), optionally wherein the dimerization molecule is an mTOR inhibitor, e.g., a rapamycin analogue, e.g., RAD001; and/or (iv) the antigen binding domain binds to a target antigen but does not promote an immune effector response of a T cell, until the dimerization molecule is present.

In some embodiments, (i) the intracellular signaling member comprises a primary intracellular signaling domain, e.g., a primary intracellular signaling domain disclosed herein, e.g., a CD3zeta domain; (ii) the intracellular signaling member comprises a costimulatory signaling domain, e.g., a costimulatory signaling domain disclosed herein, e.g., a 4-1BB domain or a CD28 domain; (iii) the antigen binding member does not comprise a primary intracellular signaling domain, e.g., the antigen binding member comprises a costimulatory signaling domain and does not comprise a primary intracellular signaling domain; (iv) the inhibitory extracellular domain member does not comprise a primary intracellular signaling domain, e.g., the inhibitory extracellular domain member comprises a costimulatory signaling domain and does not comprise a primary intracellular signaling domain; and/or (v) the costimulatory extracellular domain member does not comprise a primary intracellular signaling domain, e.g., the costimulatory extracellular domain member comprises a costimulatory signaling domain and does not comprise a primary intracellular signaling domain.

In some embodiments, the population of cells comprise the second nucleic acid molecule that encodes a CAR and a regulatory molecule.

In some embodiments, the second nucleic acid molecule comprises a nucleic acid sequence encoding the CAR and a nucleic acid sequence encoding the regulatory molecule, optionally wherein the nucleic acid sequence encoding the CAR and the nucleic acid sequence encoding the regulatory molecule are: (i) disposed on a single nucleic acid molecule, e.g., wherein the nucleic acid sequence encoding the CAR and the nucleic acid sequence encoding the regulatory molecule are separated by a nucleic acid sequence encoding a self-cleavage site; or (ii) disposed on separate nucleic acid molecules.

In some embodiments, the regulatory molecule comprises a chimeric protein comprising (i) a multimeric ligand binding region and (ii) a caspase 9 molecule. In some embodiments, the caspase 9 molecule is a truncated caspase 9, optionally wherein the caspase 9 molecule lacks the caspase recruitment domain. In some embodiments, the multimeric ligand binding region is selected from the group consisting of FKBP, cyclophilin receptor, steroid receptor, tetracycline receptor, heavy chain antibody subunit, light chain antibody subunit, single chain antibodies comprised of heavy and light chain variable regions in tandem separated by a flexible linker domain, and mutated sequences thereof, optionally wherein the multimeric ligand binding region is an FKBP12 region.

In some embodiments, the regulatory molecule comprises a truncated epidermal growth factor receptor (EGFRt). In some embodiments, the EGFRt has 1, 2, 3, 4, or all of the following properties: (i) the EGFRt comprises one or both of an EGFR Domain III and an EGFR Domain W; (ii) the EGFRt does not comprise 1, 2, 3, or all of: an EGFR Domain I, an EGFR Domain II, an EGFR juxtamembrane domain, and an EGFR tyrosine kinase domain; (iii) the EGFRt does not mediate signaling or trafficking; (iv) the EGFRt does not bind an endogenous EGFR ligand, e.g., epidermal growth factor (EGF); and (v) the EGFRt binds to an anti-EGFR-antibody molecule (e.g., cetuximab, matuzumab, necitumumab and panitumumab), an EGFR-specific siRNA, or a small molecule that targets EGFR.

In some embodiments, provide herein is a pharmaceutical composition comprising a population of cells disclosed herein and a pharmaceutically acceptable carrier.

In some embodiments, provided herein is a method of increasing an immune response in a subject, comprising administering a population of cells disclosed herein or a pharmaceutical composition disclosed herein to the subject, thereby increasing an immune response in the subject. In some embodiments, provided herein is a method of treating a cancer in a subject, comprising administering a population of cells disclosed herein or a pharmaceutical composition disclosed herein to the subject, thereby treating the cancer in the subject. In some embodiments, the cancer is a solid cancer, for example, chosen from: one or more of mesothelioma, malignant pleural mesothelioma, non-small cell lung cancer, small cell lung cancer, squamous cell lung cancer, large cell lung cancer, pancreatic cancer, pancreatic ductal adenocarcinoma, esophageal adenocarcinoma , breast cancer, glioblastoma, ovarian cancer, colorectal cancer, prostate cancer, cervical cancer, skin cancer, melanoma, renal cancer, liver cancer, brain cancer, thymoma, sarcoma, carcinoma, uterine cancer, kidney cancer, gastrointestinal cancer, urothelial cancer, pharynx cancer, head and neck cancer, rectal cancer, esophagus cancer, or bladder cancer, or a metastasis thereof. In some embodiments, the cancer is a liquid cancer, for example, chosen from: chronic lymphocytic leukemia (CLL), mantle cell lymphoma (MCL), multiple myeloma, acute lymphoid leukemia (ALL), Hodgkin lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitts lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+ large B-cell lymphoma, large B-cell lymphoma arising in HHV8-associated multicentric Castleman disease, primary effusion lymphoma, B-cell lymphoma, acute myeloid leukemia (AML), or unclassifiable lymphoma. In some embodiments, the method further comprises administering a second therapeutic agent to the subject.

In some embodiments, the method further comprises, after the administration of the population of cells or the pharmaceutical composition:

administering to the subject an effective amount of IMiD (e.g., thalidomide and derivatives thereof, e.g., lenalidomide, pomalidomide, and thalidomide) or Compound I-112. In some embodiments, the subject has developed, is developing, or is anticipated to develop an adverse reaction after the administration of the population of cells or the pharmaceutical composition. In some embodiments, the administration of IMiD or Compound I-112 is in response to an occurrence of an adverse reaction in the subject, or in response to an anticipation of an occurrence of an adverse reaction in the subject. In some embodiments, the administration of IMiD or Compound I-112 reduces or prevents an adverse effect. In some embodiments, the population of cells comprise a nucleic acid molecule that encodes a CCAR, wherein the CCAR is a fusion polypeptide comprising a degradation polypeptide (e.g., a degradation polypeptide disclosed herein) and a CAR polypeptide (e.g., a CAR polypeptide disclosed herein).

In some embodiments, provided herein is a method of treating a cancer in a subject, comprising:
i) contacting a population of cells with IMiD (e.g., thalidomide and derivatives thereof, e.g., lenalidomide, pomalidomide, and thalidomide) or Compound I-112 ex vivo, wherein the population of cells comprise a nucleic acid molecule that encodes a CCAR, wherein the CCAR is a fusion polypeptide comprising a degradation polypeptide (e.g., a degradation polypeptide disclosed herein) and a CAR polypeptide (e.g., a CAR polypeptide disclosed herein), and
ii) administering to the subject an effective amount of the population of cells, thereby treating the cancer.

In some embodiments, in the presence of IMiD or Compound I-112, the expression level of the CCAR is decreased, e.g., by at least about 1, 2, 3, 4, 5, 6, 7, 8, 9, 10, 20, 30, 40, 50, 60, 70, 80, 90, or 100 percent, relative to the expression level of the CCAR before the population of cells are contacted with IMiD or Compound I-112 ex vivo. In some embodiments, the method further comprises after step i) and prior to step ii): reducing the amount of IMiD or Compound I-112 contacting the population of cells, e.g., inside and/or surrounding the population of cells.

In some embodiments, the method further comprises after step ii):
iii) administering to the subject an effective amount of IMiD or Compound I-112. In some embodiments, the administration of IMiD or Compound I-112 decreases, e.g., by at least about 1, 2, 3, 4, 5, 6, 7, 8, 9, 10, 20, 30, 40, 50, 60, 70, 80, 90, or 100 percent, the expression level of the CCAR relative to the expression level of the CCAR after step ii) and prior to step iii). In some embodiments, the subject has developed, is developing, or is anticipated to develop an adverse reaction. In some embodiments, the administration of IMiD or Compound I-112 is in response to an occurrence of an adverse reaction in the subject, or in response to an anticipation of an occurrence of an adverse reaction in the subject. In some embodiments, the administration of IMiD or Compound I-112 reduces or prevents an adverse effect.

In some embodiments, the method further comprises after step iii):
iv) discontinuing the administration of IMiD or Compound I-112. In some embodiments, discontinuing the administration of IMiD or Compound I-112 increases, e.g., by at least about 1.5-, 2-, 3-, 4-, 5-, 10-, 20-, 30-, 40-, or 50-fold, the expression level of the CCAR relative to the expression level of the CCAR after step iii) and prior to step iv). In some embodiments, discontinuing the administration of IMiD or Compound I-112 restores the expression level of the CCAR to the expression level after step ii) and prior to step iii). In some embodiments, the subject has relapsed, is relapsing, or is anticipated to relapse. In some embodiments, the discontinuation of the administration of IMiD or Compound I-112 is in response to a tumor relapse in the subject, or in response to an anticipation of a relapse in the subject. In some embodiments, the discontinuation of the administration of IMiD or Compound I-112 treats or prevents a tumor relapse.

In some embodiments, the method further comprises after step iv):
v) repeating step iii) and/or iv), thereby treating the cancer.

In some embodiments, provided herein is a method of treating a cancer in a subject, comprising:
i) administering to the subject an effective amount of a population of cells, wherein the population of cells comprise a nucleic acid molecule that encodes a CCAR, wherein the CCAR is a fusion polypeptide comprising a degradation polypeptide (e.g., a degradation polypeptide disclosed herein) and a CAR polypeptide (e.g., a CAR polypeptide disclosed herein), thereby treating the cancer. In some embodiments, the population of cells are contacted with IMiD (e.g., thalidomide and derivatives thereof, e.g., lenalidomide, pomalidomide, and thalidomide) or Compound I-112 ex vivo before administration. In some embodiments, in the presence of IMiD or Compound I-112, the expression level of the CCAR is decreased, e.g., by at least about 1, 2, 3, 4, 5, 6, 7, 8, 9, 10, 20, 30, 40, 50, 60, 70, 80, 90, or 100 percent, relative to the expression level of the CCAR before the population of cells are contacted with IMiD or Compound I-112 ex vivo. In some embodiments, after the population of cells are contacted with IMiD or Compound I-112 ex vivo and before the population of cells are administered to the subject, the amount of IMiD or Compound I-112 contacting the population of cells, e.g., inside and/or surrounding the population of cells, is reduced.

In some embodiments, the population of cells are not contacted with IMiD or Compound I-112 ex vivo before administration.

In some embodiments, the method further comprises after step i):
ii) administering to the subject an effective amount of IMiD or Compound I-112. In some embodiments, the administration of IMiD or Compound I-112 decreases, e.g., by at least about 1, 2, 3, 4, 5, 6, 7, 8, 9, 10, 20, 30, 40, 50, 60, 70, 80, 90, or 100 percent, the expression level of the CCAR relative to the expression level of the CCAR after step i) and prior to step ii). In some embodiments, the subject has developed, is developing, or is anticipated to develop an adverse reaction. In some embodiments, the administration of IMiD or Compound I-112 is in response to an occurrence of an adverse reaction in the subject, or in response to an anticipation of an occurrence of an adverse reaction in the subject. In some embodiments, the administration of IMiD or Compound I-112 reduces or prevents an adverse effect.

In some embodiments, the method further comprises after step ii):
iii) discontinuing the administration of IMiD or Compound I-112. In some embodiments, discontinuing the administration of IMiD or Compound I-112 increases, e.g., by at least about 1.5-, 2-, 3-, 4-, 5-, 10-, 20-, 30-, 40-, or 50-fold, the expression level of the CCAR relative to the expression level of the CCAR after step ii) and prior to step iii). In some embodiments, discontinuing the administration of IMiD or Compound I-112 restores the expression level of the CCAR to the expression level after step i) and prior to step ii). In some embodiments, the subject has relapsed, is relapsing, or is anticipated to relapse. In some embodiments, the discontinuation of the administration of IMiD or Compound I-112 is in response to a tumor relapse in the subject, or in response to an anticipation of a relapse in the subject. In some embodiments, the discontinuation of the administration of IMiD or Compound I-112 treats or prevents a tumor relapse.

In some embodiments, the method further comprises after step iii):
iv) repeating step ii) and/or iii), thereby treating the cancer.

In some embodiments, provided herein is a method of treating a cancer in a subject, comprising:
i) administering an effective amount of IMiD (e.g., thalidomide and derivatives thereof, e.g., lenalidomide, pomalidomide, and thalidomide) or Compound I-112 to the subject, wherein the subject comprises a population of cells, wherein the population of cells comprise a nucleic acid molecule that encodes a CCAR, wherein the CCAR is a fusion polypeptide comprising a degradation polypeptide (e.g., a degradation polypeptide disclosed herein) and a CAR polypeptide (e.g., a CAR polypeptide disclosed herein), thereby treating the cancer. In some embodiments, the administration of IMiD or Compound I-112 decreases, e.g., by at least about 1, 2, 3, 4, 5, 6, 7, 8, 9, 10, 20, 30, 40, 50, 60, 70, 80, 90, or 100 percent, the expression level of the CCAR relative to the expression level of the CCAR before the administration of IMiD or Compound I-112. In some embodiments, the subject has developed, is developing, or is anticipated to develop an adverse reaction. In some embodiments, the administration of IMiD or Compound I-112 is in response to an occurrence of an adverse reaction in the subject, or in response to an anticipation of an occurrence of an adverse reaction in the subject. In some embodiments, the administration of IMiD or Compound I-112 reduces or prevents an adverse effect.

In some embodiments, the method further comprises after step i):
ii) discontinuing the administration of IMiD or Compound I-112. In some embodiments, discontinuing the administration of IMiD or Compound I-112 increases, e.g., by at least about 1.5-, 2-, 3-, 4-, 5-, 10-, 20-, 30-, 40-, or 50-fold, the expression level of the CCAR relative to the expression level of the CCAR after step i) and prior to step ii). In some embodiments, discontinuing the administration of IMiD or Compound I-112 restores the expression level of the CCAR to the expression level before the administration of IMiD or Compound I-112. In some embodiments, the subject has relapsed, is relapsing, or is anticipated to relapse. In some embodiments, the discontinuation of the administration of IMiD or Compound I-112 is in response to a tumor relapse in the subject, or in response to an anticipation of a relapse in the subject. In some embodiments, the discontinuation of the administration of IMiD or Compound I-112 treats or prevents a tumor relapse.

In some embodiments, the method further comprises after step ii):
iii) repeating step i) and/or ii), thereby treating the cancer.

In some embodiments, provided herein is a method of treating a cancer in a subject, comprising:
i) administering to the subject: (1) a stabilization compound, and (2) an effective amount of a population of cells, thereby treating the cancer, wherein the population of cells comprise a nucleic acid molecule that encodes a CCAR, wherein the CCAR is a fusion polypeptide comprising a degradation domain (e.g., a degradation domain disclosed herein) and a CAR polypeptide (e.g., a CAR polypeptide disclosed herein), optionally wherein the degradation domain is separated from the CAR polypeptide by a heterologous protease cleavage site. In some embodiments, the expression level of the CCAR in the presence of the stabilization compound is e.g., at least about 1.5-, 2-, 3-, 4-, 5-, 10-, 20-, 30-, 40-, or 50-fold, higher than the expression level of the CCAR in the absence of the stabilization compound.

In some embodiments, the method further comprises after step i):
ii) discontinuing the administration of the stabilization compound. In some embodiments, discontinuing the administration of the stabilization compound reduces, e.g., at least about 1.5-, 2-, 3-, 4-, 5-, 10-, 20-, 30-, 40-, or 50-fold, the expression level of the CCAR relative to the expression of the CCAR after step i) and prior to step ii). In some embodiments, the subject responded to the treatment of step i) (e.g., the subject has a complete response to the treatment of step i), the subject shows a shrinkage in tumor mass, the subject shows a decrease in tumor cells, or the treatment of step i) is effective in the subject). In some embodiments, the discontinuation of the administration of the stabilization compound is in response to a response of the subject to the treatment of step i) (e.g., the subject has a complete response to the treatment of step i), the subject shows a shrinkage in tumor mass, the subject shows a decrease in tumor cells, or the treatment of step i) is effective in the subject).

In some embodiments, the method further comprises after step i):
iii) discontinuing the administration of the stabilization compound. In some embodiments, discontinuing the administration of the stabilization compound reduces, e.g., at least about 1.5-, 2-, 3-, 4-, 5-, 10-, 20-, 30-, 40-, or 50-fold, the expression level of the CCAR relative to the expression of the CCAR after step i) and prior to step ii). In some embodiments, the subject has developed, is developing, or is anticipated to develop an adverse reaction. In some embodiments, the discontinuation of the administration of the stabilization compound is in response to an occurrence of an adverse reaction in the subject, or in response to an anticipation of an occurrence of an adverse reaction in the subject. In some embodiments, the discontinuation of the administration of the stabilization compound reduces or prevents an adverse effect.

In some embodiments, the method further comprises after step ii) or iii):
iv) administering an effective amount of a stabilization compound. In some embodiments, the administration of the stabilization compound increases, e.g., by at least about 1.5-, 2-, 3-, 4-, 5-, 10-, 20-, 30-, 40-, or 50-fold, the expression level of the CCAR relative to the expression level of the CCAR after step ii) or iii) and prior to step iv). In some embodiments, the subject has relapsed, is relapsing, or is anticipated to relapse. In some embodiments, the administration of the stabilization compound is in response to a tumor relapse in the subject, or in response to an anticipation of a relapse in the subject. In some embodiments, the administration of the stabilization compound treats or prevents a tumor relapse.

In some embodiments, the method further comprises after step iv):
v) repeating step ii), iii), or iv), thereby treating the cancer.

In some embodiments, the method further comprises prior to step i):
vi) contacting the population of cells with a stabilization compound ex vivo. In some embodiments, the expression level of the CCAR in the presence of the stabilization compound is, e.g., at least about 1.5-, 2-, 3-, 4-, 5-, 10-, 20-, 30-, 40-, or 50-fold, higher than the expression level of the CCAR in the absence of the stabilization compound.

In some embodiments, the population of cells are not contacted with the stabilization compound ex vivo before administration.

In some embodiments, provided herein is a population of cells disclosed herein or a pharmaceutical composition disclosed herein for use in a method of increasing an immune response in a subject, said method comprising administering to the subject an effective amount of the population of cells or an effective amount of the pharmaceutical composition. In some embodiments, provided herein is a population of cells disclosed herein or a pharmaceutical composition disclosed herein for use in a method of treating a cancer in a subject, said method comprising administering to the subject an effective amount of the population of cells or an effective amount of the pharmaceutical composition.

In some embodiments, this disclosure features a method of making a population of cells (for example, T cells) that express a chimeric antigen receptor (CAR), e.g., a CAR disclosed herein, e.g., a CCAR disclosed herein. In some embodiments, the population of cells further express a regulatory molecule. In some embodiments, the population of cells express a CCAR disclosed herein. In some embodiments, the population of cells express a CAR disclosed herein and a regulatory molecule disclosed herein. In some embodiments, the method comprises: (i) contacting (for example, binding) a population of cells (for example, T cells, for example, T cells isolated from a frozen or fresh leukapheresis product) with an agent that stimulates a CD3/TCR complex and/or an agent that stimulates a costimulatory molecule on the surface of the cells; (ii) contacting the population of cells (for example, T cells) with a nucleic acid molecule (for example, a DNA or RNA molecule) encoding the CAR, thereby providing a population of cells (for example, T cells) comprising the nucleic acid molecule, and (iii) harvesting the population of cells (for example, T cells) for storage (for example, reformulating the population of cells in cryopreservation media) or administration, wherein: (a) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and step (iii) is performed no later than 26 hours after the beginning of step (i), for example, no later than 22, 23, 24, or 25 hours after the beginning of step (i), for example, no later than 24 hours after the beginning of step (i); (b) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and step (iii) is performed no later than 30 hours after the beginning of step (ii), for example, no later than 22, 23, 24, 25, 26, 27, 28, 29, or 30 hours after the beginning of step (ii); or (c) the population of cells from step (iii) are not expanded, or expanded by no more than 5, 6, 7, 8, 9, 10, 11, 12, 13, 14, 15, 16, 17, 18, 19, 20, 25, 30, 35, or 40%, for example, no more than 10%, for example, as assessed by the number of living cells, compared to the population of cells at the beginning of step (i). In some embodiments, the nucleic acid molecule in step (ii) is a DNA molecule. In some embodiments, the nucleic acid molecule in step (ii) is an RNA molecule. In some embodiments, the nucleic acid molecule in step (ii) is on a viral vector, for example, a viral vector chosen from a lentivirus vector, an adenoviral vector, or a retrovirus vector. In some embodiments, the nucleic acid molecule in step (ii) is on a non-viral vector. In some embodiments, the nucleic acid molecule in step (ii) is on a plasmid. In some embodiments, the nucleic acid molecule in step (ii) is not on any vector. In some embodiments, step (ii) comprises transducing the population of cells (for example, T cells) with a viral vector comprising a nucleic acid molecule encoding the CAR. In some embodiments, step (ii) is performed together with step (i). In some embodiments, step (ii) is performed no later than 20 hours after the beginning of step (i). In some embodiments, step (ii) is performed no later than 12, 13, 14, 15, 16, 17, or 18 hours after the beginning of step (i). In some embodiments, step (ii) is performed no later than 18 hours after the beginning of step (i). In some embodiments, step (iii) is performed no later than 26 hours after the beginning of step (i). In some embodiments, step (iii) is performed no later than 22, 23, 24, or 25 hours after the beginning of step (i). In some embodiments, step (iii) is performed no later than 24 hours after the beginning of step (i). In some embodiments, step (iii) is performed no later than 30 hours after the beginning of step (ii). In some embodiments, step (iii) is performed no later than 22, 23, 24, 25, 26, 27, 28, 29, or 30 hours after the beginning of step (ii).

In some embodiments, the population of cells from step (iii) are not expanded. In some embodiments, the population of cells from step (iii) are expanded by no more than 5, 6, 7, 8, 9, 10, 11, 12, 13, 14, 15, 16, 17, 18, 19, 20, 25, 30, 35, or 40%, for example, as assessed by the number of living cells, compared to the population of cells at the beginning of step (i). In some embodiments, the population of cells from step (iii) are expanded by no more than 10%, for example, as assessed by the number of living cells, compared to the population of cells at the beginning of step (i).

In some embodiments, the agent that stimulates a CD3/TCR complex is an agent that stimulates CD3. In some embodiments, the agent that stimulates a costimulatory molecule is an agent that stimulates CD28, ICOS, CD27, HVEM, LIGHT, CD40, 4-1BB, OX40, DR3, GITR, CD30, TIM1, CD2, CD226, or any combination thereof. In some embodiments, the agent that stimulates a costimulatory molecule is an agent that stimulates CD28. In some embodiments, the agent that stimulates a CD3/TCR complex is chosen from an antibody (for example, a single-domain antibody (for example, a heavy chain variable domain antibody), a peptibody, a Fab fragment, or a scFv), a small molecule, or a ligand (for example, a naturally-existing, recombinant, or chimeric ligand). In some embodiments, the agent that stimulates a costimulatory molecule is chosen from an antibody (for example, a single-domain antibody (for example, a heavy chain variable domain antibody), a peptibody, a Fab fragment, or a scFv), a small molecule, or a ligand (for example, a naturally-existing, recombinant, or chimeric ligand). In some embodiments, the agent that stimulates a CD3/TCR complex does not comprise a bead. In some embodiments, the agent that stimulates a costimulatory molecule does not comprise a bead. In some embodiments, the agent that stimulates a CD3/TCR complex comprises an anti-CD3 antibody. In some embodiments, the agent that stimulates a costimulatory molecule comprises an anti-CD28 antibody. In some embodiments, the agent that stimulates a CD3/TCR complex comprises an anti-CD3 antibody covalently attached to a colloidal polymeric nanomatrix. In some embodiments, the agent that stimulates a costimulatory molecule comprises an anti-CD28 antibody covalently attached to a colloidal polymeric nanomatrix. In some embodiments, the agent that stimulates a CD3/TCR complex and the agent that stimulates a costimulatory molecule comprise T Cell TransAct™.

In some embodiments, the agent that stimulates a CD3/TCR complex does not comprise hydrogel. In some embodiments, the agent that stimulates a costimulatory molecule does not comprise hydrogel. In some embodiments, the agent that stimulates a CD3/TCR complex does not comprise alginate. In some embodiments, the agent that stimulates a costimulatory molecule does not comprise alginate.

In some embodiments, the agent that stimulates a CD3/TCR complex comprises hydrogel. In some embodiments, the agent that stimulates a costimulatory molecule comprises hydrogel. In some embodiments, the agent that stimulates a CD3/TCR complex comprises alginate. In some embodiments, the agent that stimulates a costimulatory molecule comprises alginate. In some embodiments, the agent that stimulates a CD3/TCR complex or the agent that stimulates a costimulatory molecule comprises MagCloudz™ from Quad Technologies.

In some embodiments, step (i) increases the percentage of CAR-expressing cells in the population of cells from step (iii), for example, the population of cells from step (iii) shows a higher percentage of CAR-expressing cells (for example, at least 10, 20, 30, 40, 50, or 60% higher), compared with cells made by an otherwise similar method without step (i).

In some embodiments, the percentage of naïve cells, for example, naïve T cells, for example, CD45RA+ CD45RO− CCR7+ T cells, in the population of cells from step (iii) is the same as the percentage of naïve cells, for example, naïve T cells, for example, CD45RA+ CD45RO− CCR7+ cells, in the population of cells at the beginning of step (i). In some embodiments, the percentage of naïve cells, for example, naïve T cells, for example, CD45RA+ CD45RO− CCR7+ T cells, in the population of cells from step (iii) differs by no more than 3, 4, 5, 6, 7, 8, 9, 10, 11, or 12% from the percentage of naïve cells, for example, naïve T cells, for example, CD45RA+ CD45RO− CCR7+ cells, in the population of cells at the beginning of step (i). In some embodiments, the percentage of naïve cells, for example, naïve T cells, for example, CD45RA+ CD45RO− CCR7+ T cells, in the population of cells from step (iii) differs by no more than 5 or 10% from the percentage of naïve cells, for example, naïve T cells, for example, CD45RA+ CD45RO− CCR7+ cells, in the population of cells at the beginning of step (i).

In some embodiments, the population of cells from step (iii) shows a higher percentage of naïve cells, for example, naïve T cells, for example, CD45RA+ CD45RO− CCR7+ T cells (for example, at least 10, 11, 12, 13, 14, 15, 16, 17, 18, 19, 20, 25, 30, 35, or 40% higher), compared with cells made by an otherwise similar method in which step (iii) is performed more than 26 hours after the beginning of step (i), for example, more than 5, 6, 7, 8, 9, 10, 11, or 12 days after the beginning of step (i). In some embodiments, the population of cells from step (iii) shows a higher percentage of naïve cells, for example, naïve T cells, for example, CD45RA+ CD45RO− CCR7+ T cells (for example, at least 10, 11, 12, 13, 14, 15, 16, 17, 18, 19, 20, 25, 30, 35, or 40% higher), compared with cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the percentage of central memory cells, for example, central memory T cells, for example, CD95+ central memory T cells, in the population of cells from step (iii) is the same as the percentage of central memory cells, for example, central memory T cells, for example, CD95+ central memory T cells, in the population of cells at the beginning of step (i). In some embodiments, the percentage of central memory cells, for example, central memory T cells, for example, CD95+ central memory T cells, in the population of cells from step (iii) differs by no more than 3, 4, 5, 6, 7, 8, 9, 10, 11, or 12% from the percentage of central memory cells, for example, central memory T cells, for example, CD95+ central memory T cells, in the population of cells at the beginning of step (i). In some embodiments, the percentage of central memory cells, for example, central memory T cells, for example, CD95+ central memory T cells, in the population of cells from step (iii) differs by no more than 5 or 10% from the percentage of central memory cells, for example, central memory T cells, for example, CD95+ central memory T cells, in the population of cells at the beginning of step (i).

In some embodiments, the population of cells from step (iii) shows a lower percentage of central memory cells, for example, central memory T cells, for example, CD95+ central memory T cells (for example, at least 10, 11, 12, 13, 14, 15, 16, 17, 18, 19, 20, 25, 30, 35, or 40% lower), compared with cells made by an otherwise similar method in which step (iii) is performed more than 26 hours after the beginning of step (i), for example, more than 5, 6, 7, 8, 9, 10, 11, or 12 days after the beginning of step (i). In some embodiments, the population of cells from step (iii) shows a lower percentage of central memory cells, for example, central memory T cells, for example, CD95+ central memory T cells (for example, at least 10, 11, 12, 13, 14, 15, 16, 17, 18, 19, 20, 25, 30, 35, or 40% lower), compared with cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the percentage of stem memory T cells, for example, CD45RA+CD95+IL-2 receptor β+CCR7+CD62L+ T cells, in the population of cells from step (iii) is increased, as compared to the percentage of stem memory T cells, for example, CD45RA+CD95+IL-2 receptor β+CCR7+CD62L+ T cells, in the population of cells at the beginning of step (i). In some embodiments, the percentage of CAR-expressing stem memory T cells, for example, CAR-expressing CD45RA+CD95+IL-2 receptor β+CCR7+CD62L+ T cells, in the population of cells from step (iii) is increased, as compared to the percentage of CAR-expressing stem memory T cells, for example, CAR-expressing CD45RA+CD95+IL-2 receptor β+CCR7+CD62L+ T cells, in the population of cells at the beginning of step (i). In some embodiments, the percentage of stem memory T cells, for example, CD45RA+CD95+IL-2 receptor β+CCR7+CD62L+ T cells, in the population of cells from step (iii) is higher than the percentage of stem memory T cells, for example, CD45RA+CD95+IL-2 receptor β+CCR7+CD62L+ T cells, in cells made by an otherwise similar method in which step (iii) is performed more than 26 hours after the beginning of step (i), for example, more than 5, 6, 7, 8, 9, 10, 11, or 12 days after the beginning of step (i). In some embodiments, the percentage of CAR-expressing stem memory T cells, for example, CAR-expressing CD45RA+CD95+IL-2 receptor β+CCR7+CD62L+ T cells, in the population of cells from step (iii) is higher than the percentage of CAR-expressing stem memory T cells, for example, CAR-expressing CD45RA+CD95+IL-2 receptor β+CCR7+CD62L+ T cells, in cells made by an otherwise similar method in which step (iii) is performed more than 26 hours after the beginning of step (i), for example, more than 5, 6, 7, 8, 9, 10, 11, or 12 days after the beginning of step (i). In some embodiments, the percentage of stem memory T cells, for example, CD45RA+CD95+IL-2 receptor β+CCR7+CD62L+ T cells, in the population of cells from step (iii) is higher than the percentage of stem memory T cells, for example, CD45RA+CD95+IL-2 receptor β+CCR7+CD62L+ T cells, in cells made by an otherwise similar method which further comprises, after step (ii) and prior to step (iii), expanding the population of cells (for example, T cells) in vitro for more than 3 days, for example, for 5, 6, 7, 8 or 9 days. In some embodiments, the percentage of CAR-expressing stem memory T cells, for example, CAR-expressing CD45RA+CD95+IL-2 receptor β+CCR7+CD62L+ T cells, in the population of cells from step (iii) is higher than the percentage of CAR-expressing stem memory T cells, for example, CAR-expressing CD45RA+CD95+IL-2 receptor β+CCR7+CD62L+ T cells, in cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the median GeneSetScore (Up TEM vs. Down TSCM) of the population of cells from step (iii) is about the same as or differs by no more than (for example, increased by no more than) about 25, 50, 75, 100, or 125% from the median GeneSetScore (Up TEM vs. Down TSCM) of the population of cells at the beginning of step (i). In some embodiments, the median GeneSetScore (Up TEM vs. Down TSCM) of the population of cells from step (iii) is lower (for example, at least about 100, 150, 200, 250, or 300% lower) than the median GeneSetScore (Up TEM vs. Down TSCM) of cells made by an otherwise similar method in which step (iii) is performed more than 26 hours after the beginning of step (i), for example, more than 5, 6, 7, 8, 9, 10, 11, or 12 days after the beginning of step (i). In some embodiments, the median GeneSetScore (Up TEM vs. Down TSCM) of the population of cells from step (iii) is lower (for example, at least about 100, 150, 200, 250, or 300% lower) than the median GeneSetScore (Up TEM vs. Down TSCM) of cells made by an otherwise similar method which further comprises, after step (ii) and prior to step (iii), expanding the population of cells (for example, T cells) in vitro for more than 3 days, for example, for 5, 6, 7, 8 or 9 days. In some embodiments, the median GeneSetScore (Up Treg vs. Down Teff) of the population of cells from step (iii) is about the same as or differs by no more than (for example, increased by no more than) about 25, 50, 100, 150, or 200% from the median GeneSetScore (Up Treg vs. Down Teff) of the population of cells at the beginning of step (i). In some embodiments, the median GeneSetScore (Up Treg vs. Down Teff) of the population of cells from step (iii) is lower (for example, at least about 50, 100, 125, 150, or 175% lower) than the median GeneSetScore (Up Treg vs. Down Teff) of cells made by an otherwise similar method in which step (iii) is performed more than 26 hours after the beginning of step (i), for example, more than 5, 6, 7, 8, 9, 10, 11, or 12 days after the beginning of step (i). In some embodiments, the median GeneSetScore (Up Treg vs. Down Teff) of the population of cells from step (iii) is lower (for example, at least about 50, 100, 125, 150, or 175% lower) than the median GeneSetScore (Up Treg vs. Down Teff) of cells made by an otherwise similar method which further comprises, after step (ii) and prior to step (iii), expanding the population of cells (for example, T cells) in vitro for more than 3 days, for example, for 5, 6, 7, 8 or 9 days. In some embodiments, the median GeneSetScore (Down stemness) of the population of cells from step (iii) is about the same as or differs by no more than (for example, increased by no more than) about 25, 50, 100, 150, 200, or 250% from the median GeneSetScore (Down stemness) of the population of cells at the beginning of step (i). In some embodiments, the median GeneSetScore (Down stemness) of the population of cells from step (iii) is lower (for example, at least about 50, 100, or 125% lower) than the median GeneSetScore (Down stemness) of cells made by an otherwise similar method in which step (iii) is performed more than 26 hours after the beginning of step (i), for example, more than 5, 6, 7, 8, 9, 10, 11, or 12 days after the beginning of step (i). In some embodiments, the median GeneSetScore (Down stemness) of the population of cells from step (iii) is lower (for example, at least about 50, 100, or 125% lower) than the median GeneSetScore (Down stemness) of cells made by an otherwise similar method which further comprises, after step (ii) and prior to step (iii), expanding the population of cells (for example, T cells) in vitro for more than 3 days, for example, for 5, 6, 7, 8 or 9 days. In some embodiments, the median GeneSetScore (Up hypoxia) of the population of cells from step (iii) is about the same as or differs by no more than (for example, increased by no more than) about 125, 150, 175, or 200% from the median GeneSetScore (Up hypoxia) of the population of cells at the beginning of step (i). In some embodiments, the median GeneSetScore (Up hypoxia) of the population of cells from step (iii) is lower (for example, at least about 40, 50, 60, 70, or 80% lower) than the median GeneSetScore (Up hypoxia) of cells made by an otherwise similar method in which step (iii) is performed more than 26 hours after the beginning of step (i), for example, more than 5, 6, 7, 8, 9, 10, 11, or 12 days after the beginning of step (i). In some embodiments, the median GeneSetScore (Up hypoxia) of the population of cells from step (iii) is lower (for example, at least about 40, 50, 60, 70, or 80% lower) than the median GeneSetScore (Up hypoxia) of cells made by an otherwise similar method which further comprises, after step (ii) and prior to step (iii), expanding the population of cells (for example, T cells) in vitro for more than 3 days, for example, for 5, 6, 7, 8 or 9 days. In some embodiments, the median GeneSetScore (Up autophagy) of the population of cells from step (iii) is about the same as or differs by no more than (for example, increased by no more than) about 180, 190, 200, or 210% from the median GeneSetScore (Up autophagy) of the population of cells at the beginning of step (i). In some embodiments, the median GeneSetScore (Up autophagy) of the population of cells from step (iii) is lower (for example, at least 20, 30, or 40% lower) than the median GeneSetScore (Up autophagy) of cells made by an otherwise similar method in which step (iii) is performed more than 26 hours after the beginning of step (i), for example, more than 5, 6, 7, 8, 9, 10, 11, or 12 days after the beginning of step (i). In some embodiments, the median GeneSetScore (Up autophagy) of the population of cells from step (iii) is lower (for example, at least 20, 30, or 40% lower) than the median GeneSetScore (Up autophagy) of cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the population of cells from step (iii), after being incubated with a cell expressing an antigen recognized by the CAR, secretes IL-2 at a higher level (for example, at least 2, 4, 6, 8, 10, 12, or 14-fold higher) than cells made by an otherwise similar method in which step (iii) is performed more than 26 hours after the beginning of step (i), for example, more than 5, 6, 7, 8, 9, 10, 11, or 12 days after the beginning of step (i), or cells made by an otherwise similar method which further comprises, after step (ii) and prior to step (iii), expanding the population of cells (for example, T cells) in vitro for more than 3 days, for example, for 5, 6, 7, 8 or 9 days, for example, as assessed using methods described in Example 8 with respect to FIGS. 29C-29D.

In some embodiments, the population of cells from step (iii), after being administered in vivo, persists longer or expands at a higher level (for example, at least 20, 25, 30, 35, 40, 45, 50, 55, 60, 65, 70, 75, 80, 85, or 90% higher) (for example, as assessed using methods described in Example 1 with respect to FIG. 4C), compared with cells made by an otherwise similar method in which step (iii) is performed more than 26 hours after the beginning of step (i), for example, more than 5, 6, 7, 8, 9, 10, 11, or 12 days after the beginning of step (i). In some embodiments, the population of cells from step (iii), after being administered in vivo, persists longer or expands at a higher level (for example, at least 20, 25, 30, 35, 40, 45, 50, 55, 60, 65, 70, 75, 80, 85, or 90% higher) (for example, as assessed using methods described in Example 1 with respect to FIG. 4C), compared with cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the population of cells from step (iii), after being administered in vivo, shows a stronger anti-tumor activity (for example, a stronger anti-tumor activity at a low dose, for example, a dose no more than 0.15×10⁶, 0.2×10⁶, 0.25×10⁶, or 0.3×10⁶ viable CAR-expressing cells) than cells made by an otherwise similar method in which step (iii) is performed more than 26 hours after the beginning of step (i), for example, more than 5, 6, 7, 8, 9, 10, 11, or 12 days after the beginning of step (i), or cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the population of cells from step (iii) are not expanded, for example, as assessed by the number of living cells, compared to the population of cells at the beginning of step (i). In some embodiments, the population of cells from step (iii) decreases from the number of living cells in the population of cells at the beginning of step (i), for example, as assessed by the number of living cells. In some embodiments, the population of cells from step (iii) are expanded by no more than 5, 6, 7, 8, 9, 10, 11, 12, 13, 14, 15, 16, 17, 18, 19, 20, 25, 30, 35, or 40%, for example, no more than 10%, for example, as assessed by the number of living cells, compared to the population of cells at the beginning of step (i). In some embodiments, the population of cells from step (iii) are not expanded, or expanded by less than 0.5, 1, 1.5, or 2 hours, for example, less than 1 or 1.5 hours, compared to the population of cells at the beginning of step (i).

In some embodiments, steps (i) and (ii) are performed in cell media (for example, serum-free media) comprising IL-2, IL-15 (for example, hetIL-15 (IL15/sIL-15Ra)), IL-6 (for example, IL-6/sIL-6Ra), a LSD1 inhibitor, or a MALT1 inhibitor. In some embodiments, steps (i) and (ii) are performed in cell media (for example, serum-free media) comprising IL-7, IL-21, or a combination thereof. In some embodiments, steps (i) and (ii) are performed in cell media (for example, serum-free media) comprising IL-2, IL-15 (for example, hetIL-15 (IL15/sIL-15Ra)), IL-21, IL-7, IL-6 (for example, IL-6/sIL-6Ra), a LSD1 inhibitor, a MALT1 inhibitor, or a combination thereof. In some embodiments, step (i) is performed in cell media (for example, serum-free media) comprising IL-2, IL-15 (for example, hetIL-15 (IL15/sIL-15Ra)), IL-6 (for example, IL-6/sIL-6Ra), a LSD1 inhibitor, or a MALT1 inhibitor. In some embodiments, step (ii) is performed in cell media (for example, serum-free media) comprising IL-2, IL-15 (for example, hetIL-15 (IL15/sIL-15Ra)), IL-6 (for example, IL-6/sIL-6Ra), a LSD1 inhibitor, or a MALT1 inhibitor. In some embodiments, step (i) is performed in cell media (for example, serum-free media) comprising IL-7, IL-21, or a combination thereof. In some embodiments, step (ii) is performed in cell media (for example, serum-free media) comprising IL-7, IL-21, or a combination thereof. In some embodiments, step (i) is performed in cell media (for example, serum-free media) comprising IL-2, IL-15 (for example, hetIL-15 (IL15/sIL-15Ra)), IL-21, IL-7, IL-6 (for example, IL-6/sIL-6Ra), a LSD1 inhibitor, a MALT1 inhibitor, or a combination thereof. In some embodiments, step (ii) is performed in cell media (for example, serum-free media) comprising IL-2, IL-15 (for example, hetIL-15 (IL15/sIL-15Ra)), IL-21, IL-7, IL-6 (for example, IL-6/sIL-6Ra), a LSD1 inhibitor, a MALT1 inhibitor, or a combination thereof. In some embodiments, the cell media is a serum-free media comprising a serum replacement. In some embodiments, the serum replacement is CTSTM Immune Cell Serum Replacement (ICSR).

In some embodiments, the aforementioned methods further comprise prior to step (i): (iv) receiving a fresh leukapheresis product (or an alternative source of hematopoietic tissue such as a fresh whole blood product, a fresh bone marrow product, or a fresh tumor or organ biopsy or removal (for example, a fresh product from thymectomy)) from an entity, for example, a laboratory, hospital, or healthcare provider.

In some embodiments, the aforementioned methods further comprise prior to step (i): (v) isolating the population of cells (for example, T cells, for example, CD8+ and/or CD4+ T cells) contacted in step (i) from a fresh leukapheresis product (or an alternative source of hematopoietic tissue such as a fresh whole blood product, a fresh bone marrow product, or a fresh tumor or organ biopsy or removal (for example, a fresh product from thymectomy)). In some embodiments, step (iii) is performed no later than hours after the beginning of step (v), for example, no later than 27, 28, 29, 30, 31, 32, 33, 34, or 35 hours after the beginning of step (v), for example, no later than 30 hours after the beginning of step (v). In some embodiments, the population of cells from step (iii) are not expanded, or expanded by no more than 5, 10, 15, 20, 25, 30, 35, or 40%, for example, no more than 10%, for example, as assessed by the number of living cells, compared to the population of cells at the end of step (v).

In some embodiments, the aforementioned methods further comprise prior to step (i): receiving cryopreserved T cells isolated from a leukapheresis product (or an alternative source of hematopoietic tissue such as cryopreserved T cells isolated from whole blood, bone marrow, or tumor or organ biopsy or removal (for example, thymectomy)) from an entity, for example, a laboratory, hospital, or healthcare provider.

In some embodiments, the aforementioned methods further comprise prior to step (i): (iv) receiving a cryopreserved leukapheresis product (or an alternative source of hematopoietic tissue such as a cryopreserved whole blood product, a cryopreserved bone marrow product, or a cryopreserved tumor or organ biopsy or removal (for example, a cryopreserved product from thymectomy)) from an entity, for example, a laboratory, hospital, or healthcare provider.

In some embodiments, the aforementioned methods further comprise prior to step (i): (v) isolating the population of cells (for example, T cells, for example, CD8+ and/or CD4+ T cells) contacted in step (i) from a cryopreserved leukapheresis product (or an alternative source of hematopoietic tissue such as a cryopreserved whole blood product, a cryopreserved bone marrow product, or a cryopreserved tumor or organ biopsy or removal (for example, a cryopreserved product from thymectomy)). In some embodiments, step (iii) is performed no later than 35 hours after the beginning of step (v), for example, no later than 27, 28, 29, 30, 31, 32, 33, 34, or 35 hours after the beginning of step (v), for example, no later than 30 hours after the beginning of step (v). In some embodiments, the population of cells from step (iii) are not expanded, or expanded by no more than 5, 10, 15, 20, 25, 30, 35, or 40%, for example, no more than 10%, for example, as assessed by the number of living cells, compared to the population of cells at the end of step (v).

In some embodiments, this disclosure features a method of making a population of cells (for example, T cells) that express a chimeric antigen receptor (CAR), e.g., a CAR disclosed herein, e.g., a CCAR disclosed herein. In some embodiments, the population of cells further express a regulatory molecule. In some embodiments, the population of cells express a CCAR disclosed herein. In some embodiments, the population of cells express a CAR disclosed herein and a regulatory molecule disclosed herein. In some embodiments, the method comprises: (1) contacting a population of cells (for example, T cells, for example, T cells isolated from a frozen leukapheresis product) with a cytokine chosen from IL-2, IL-7, IL-15, IL-21, IL-6, or a combination thereof, (2) contacting the population of cells (for example, T cells) with a nucleic acid molecule (for example, a DNA or RNA molecule) encoding the CAR, thereby providing a population of cells (for example, T cells) comprising the nucleic acid molecule, and (3) harvesting the population of cells (for example, T cells) for storage (for example, reformulating the population of cells in cryopreservation media) or administration, wherein: (a) step (2) is performed together with step (1) or no later than 5 hours after the beginning of step (1), for example, no later than 1, 2, 3, 4, or 5 hours after the beginning of step (1), and step (3) is performed no later than 26 hours after the beginning of step (1), for example, no later than 22, 23, 24, or 25 hours after the beginning of step (1), for example, no later than 24 hours after the beginning of step (1), or (b) the population of cells from step (3) are not expanded, or expanded by no more than 5, 10, 15, 20, 25, 30, 35, or 40%, for example, no more than 10%, for example, as assessed by the number of living cells, compared to the population of cells at the beginning of step (1). In some embodiments, the nucleic acid molecule in step (2) is a DNA molecule. In some embodiments, the nucleic acid molecule in step (2) is an RNA molecule. In some embodiments, the nucleic acid molecule in step (2) is on a viral vector, for example, a viral vector chosen from a lentivirus vector, an adenoviral vector, or a retrovirus vector. In some embodiments, the nucleic acid molecule in step (2) is on a non-viral vector. In some embodiments, the nucleic acid molecule in step (2) is on a plasmid. In some embodiments, the nucleic acid molecule in step (2) is not on any vector. In some embodiments, step (2) comprises transducing the population of cells (for example, T cells) with a viral vector comprising a nucleic acid molecule encoding the CAR.

In some embodiments, step (2) is performed together with step (1). In some embodiments, step (2) is performed no later than 5 hours after the beginning of step (1). In some embodiments, step (2) is performed no later than 1, 2, 3, 4, or 5 hours after the beginning of step (1). In some embodiments, step (3) is performed no later than 26 hours after the beginning of step (1). In some embodiments, step (3) is performed no later than 22, 23, 24, or 25 hours after the beginning of step (1). In some embodiments, step (3) is performed no later than 24 hours after the beginning of step (1).

In some embodiments, the population of cells from step (3) are not expanded, for example, as assessed by the number of living cells, compared to the population of cells at the beginning of step (1). In some embodiments, the population of cells from step (3) are expanded by no more than 5, 6, 7, 8, 9, 10, 11, 12, 13, 14, 15, 16, 17, 18, 19, 20, 25, 30, 35, or 40%, for example, as assessed by the number of living cells, compared to the population of cells at the beginning of step (1). In some embodiments, the population of cells from step (3) are expanded by no more than 10%, for example, as assessed by the number of living cells, compared to the population of cells at the beginning of step (1).

In some embodiments, step (1) comprises contacting the population of cells (for example, T cells) with IL-2. In some embodiments, step (1) comprises contacting the population of cells (for example, T cells) with IL-7. In some embodiments, step (1) comprises contacting the population of cells (for example, T cells) with IL-15 (for example, hetIL-15 (IL15/sIL-15Ra)). In some embodiments, step (1) comprises contacting the population of cells (for example, T cells) with IL-21. In some embodiments, step (1) comprises contacting the population of cells (for example, T cells) with IL-6 (for example, IL-6/sIL-6Ra). In some embodiments, step (1) comprises contacting the population of cells (for example, T cells) with IL-2 and IL-7. In some embodiments, step (1) comprises contacting the population of cells (for example, T cells) with IL-2 and IL-15 (for example, hetIL-15 (IL15/sIL-15Ra)). In some embodiments, step (1) comprises contacting the population of cells (for example, T cells) with IL-2 and IL-21. In some embodiments, step (1) comprises contacting the population of cells (for example, T cells) with IL-2 and IL-6 (for example, IL-6/sIL-6Ra). In some embodiments, step (1) comprises contacting the population of cells (for example, T cells) with IL-7 and IL-15 (for example, hetIL-15 (IL15/sIL-15Ra)). In some embodiments, step (1) comprises contacting the population of cells (for example, T cells) with IL-7 and IL-21. In some embodiments, step (1) comprises contacting the population of cells (for example, T cells) with IL-7 and IL-6 (for example, IL-6/sIL-6Ra). In some embodiments, step (1) comprises contacting the population of cells (for example, T cells) with IL-15 (for example, hetIL-15 (IL15/sIL-15Ra)) and IL-21. In some embodiments, step (1) comprises contacting the population of cells (for example, T cells) with IL-15 (for example, hetIL-15 (IL15/sIL-15Ra)) and IL-6 (for example, IL-6/sIL-6Ra). In some embodiments, step (1) comprises contacting the population of cells (for example, T cells) with IL-21 and IL-6 (for example, IL-6/sIL-6Ra). In some embodiments, step (1) comprises contacting the population of cells (for example, T cells) with IL-7, IL-15 (for example, hetIL-15 (IL15/sIL-15Ra)), and IL-21.

In some embodiments, the population of cells from step (3) shows a higher percentage of naïve cells among CAR-expressing cells (for example, at least 10, 11, 12, 13, 14, 15, 16, 17, 18, 19, 20, 25, 30, 35, or 40% higher), compared with cells made by an otherwise similar method which further comprises contacting the population of cells with, for example, an anti-CD3 antibody.

In some embodiments, the percentage of naïve cells, for example, naïve T cells, for example, CD45RA+ CD45RO− CCR7+ T cells, in the population of cells from step (3) is the same as the percentage of naïve cells, for example, naïve T cells, for example, CD45RA+ CD45RO− CCR7+ cells, in the population of cells at the beginning of step (1). In some embodiments, the percentage of naïve cells, for example, naïve T cells, for example, CD45RA+ CD45RO− CCR7+ T cells, in the population of cells from step (3) differs by no more than 3, 4, 5, 6, 7, 8, 9, 10, 11, or 12% from the percentage of naïve cells, for example, naïve T cells, for example, CD45RA+ CD45RO− CCR7+ cells, in the population of cells at the beginning of step (1). In some embodiments, the percentage of naïve cells, for example, naïve T cells, for example, CD45RA+ CD45RO− CCR7+ T cells, in the population of cells from step (3) differs by no more than 5 or 10% from the percentage of naïve cells, for example, naïve T cells, for example, CD45RA+ CD45RO− CCR7+ cells, in the population of cells at the beginning of step (1). In some embodiments, the percentage of naïve cells, for example, naïve T cells, for example, CD45RA+ CD45RO− CCR7+ T cells, in the population of cells from step (3) is increased as compared to the percentage of naïve cells, for example, naïve T cells, for example, CD45RA+ CD45RO− CCR7+ cells, in the population of cells at the beginning of step (1). In some embodiments, the percentage of naïve cells, for example, naïve T cells, for example, CD45RA+ CD45RO− CCR7+ T cells, in the population of cells from step (3) is increased by at least 10, 11, 12, 13, 14, 15, 16, 17, 18, 19, or 20%, as compared to the percentage of naïve cells, for example, naïve T cells, for example, CD45RA+ CD45RO− CCR7+ cells, in the population of cells at the beginning of step (1). In some embodiments, the percentage of naïve cells, for example, naïve T cells, for example, CD45RA+ CD45RO− CCR7+ T cells, in the population of cells from step (3) is increased by at least 10 or 20%, as compared to the percentage of naïve cells, for example, naïve T cells, for example, CD45RA+ CD45RO− CCR7+ cells, in the population of cells at the beginning of step (1).

In some embodiments, the population of cells from step (3) shows a higher percentage of naïve cells, for example, naïve T cells, for example, CD45RA+ CD45RO− CCR7+ T cells (for example, at least 10, 11, 12, 13, 14, 15, 16, 17, 18, 19, 20, 25, 30, 35, or 40% higher), compared with cells made by an otherwise similar method in which step (3) is performed more than 26 hours after the beginning of step (1), for example, more than 5, 6, 7, 8, 9, 10, 11, or 12 days after the beginning of step (1). In some embodiments, the population of cells from step (3) shows a higher percentage of naïve cells, for example, naïve T cells, for example, CD45RA+ CD45RO− CCR7+ T cells (for example, at least 10, 11, 12, 13, 14, 15, 16, 17, 18, 19, 20, 25, 30, 35, or 40% higher), compared with cells made by an otherwise similar method which further comprises, after step (2) and prior to step (3), expanding the population of cells (for example, T cells) in vitro for more than 3 days, for example, for 5, 6, 7, 8 or 9 days.

In some embodiments, the percentage of central memory cells, for example, central memory T cells, for example, CD95+ central memory T cells, in the population of cells from step (3) is the same as the percentage of central memory cells, for example, central memory T cells, for example, CD95+ central memory T cells, in the population of cells at the beginning of step (i). In some embodiments, the percentage of central memory cells, for example, central memory T cells, for example, CD95+ central memory T cells, in the population of cells from step (3) differs by no more than 3, 4, 5, 6, 7, 8, 9, 10, 11, or 12% from the percentage of central memory cells, for example, central memory T cells, for example, CD95+ central memory T cells, in the population of cells at the beginning of step (i). In some embodiments, the percentage of central memory cells, for example, central memory T cells, for example, CD95+ central memory T cells, in the population of cells from step (3) differs by no more than 5 or 10% from the percentage of central memory cells, for example, central memory T cells, for example, CD95+ central memory T cells, in the population of cells at the beginning of step (i). In some embodiments, the percentage of central memory cells, for example, central memory T cells, for example, CD95+ central memory T cells, in the population of cells from step (3) is decreased as compared to the percentage of central memory cells, for example, central memory T cells, for example, CD95+ central memory T cells, in the population of cells at the beginning of step (1). In some embodiments, the percentage of central memory cells, for example, central memory T cells, for example, CD95+ central memory T cells, in the population of cells from step (3) is decreased by at least 10 or 20%, as compared to the percentage of central memory cells, for example, central memory T cells, for example, CD95+ central memory T cells, in the population of cells at the beginning of step (1). In some embodiments, the percentage of central memory cells, for example, central memory T cells, for example, CD95+ central memory T cells, in the population of cells from step (3) is decreased by at least 10, 11, 12, 13, 14, 15, 16, 17, 18, 19, or 20%, as compared to the percentage of central memory cells, for example, central memory T cells, for example, CD95+ central memory T cells, in the population of cells at the beginning of step (1).

In some embodiments, the population of cells from step (3) shows a lower percentage of central memory cells, for example, central memory T cells, for example, CD95+ central memory T cells (for example, at least 10, 11, 12, 13, 14, 15, 16, 17, 18, 19, 20, 25, 30, 35, or 40% lower), compared with cells made by an otherwise similar method in which step (3) is performed more than 26 hours after the beginning of step (1), for example, more than 5, 6, 7, 8, 9, 10, 11, or 12 days after the beginning of step (1). In some embodiments, the population of cells from step (3) shows a lower percentage of central memory cells, for example, central memory T cells, for example, CD95+ central memory T cells (for example, at least 10, 11, 12, 13, 14, 15, 16, 17, 18, 19, 20, 25, 30, 35, or 40% lower), compared with cells made by an otherwise similar method which further comprises, after step (2) and prior to step (3), expanding the population of cells (for example, T cells) in vitro for more than 3 days, for example, for 5, 6, 7, 8 or 9 days.

In some embodiments, the population of cells from step (3), after being administered in vivo, persists longer or expands at a higher level (for example, at least 20, 25, 30, 35, 40, 45, 50, 55, 60, 65, 70, 75, 80, 85, or 90% higher) (for example, as assessed using methods described in Example 1 with respect to FIG. 4C), compared with cells made by an otherwise similar method in which step (3) is performed more than 26 hours after the beginning of step (1), for example, more than 5, 6, 7, 8, 9, 10, 11, or 12 days after the beginning of step (1). In some embodiments, the population of cells from step (3), after being administered in vivo, persists longer or expands at a higher level (for example, at least 20, 25, 30, 35, 40, 45, 50, 55, 60, 65, 70, 75, 80, 85, or 90% higher) (for example, as assessed using methods described in Example 1 with respect to FIG. 4C), compared with cells made by an otherwise similar method which further comprises, after step (2) and prior to step (3), expanding the population of cells (for example, T cells) in vitro for more than 3 days, for example, for 5, 6, 7, 8 or 9 days.

In some embodiments, the population of cells from step (3) are not expanded, for example, as assessed by the number of living cells, compared to the population of cells at the beginning of step (1). In some embodiments, the population of cells from step (3) are expanded by no more than 5, 6, 7, 8, 9, 10, 11, 12, 13, 14, 15, 16, 17, 18, 19, 20, 25, 30, 35, or 40%, for example, as assessed by the number of living cells, compared to the population of cells at the beginning of step (1). In some embodiments, the population of cells from step (3) are expanded by no more than 10%, for example, as assessed by the number of living cells, compared to the population of cells at the beginning of step (1). In some embodiments, the number of living cells in the population of cells from step (3) decreases from the number of living cells in the population of cells at the beginning of step (1), for example, as assessed by the number of living cells.

In some embodiments, the population of cells from step (3) are not expanded compared to the population of cells at the beginning of step (1), for example, as assessed by the number of living cells. In some embodiments, the population of cells from step (3) are expanded by less than 0.5, 1, 1.5, or 2 hours, for example, less than 1 or 1.5 hours, compared to the population of cells at the beginning of step (1).

In some embodiments, the population of cells is not contacted in vitro with an agent that stimulates a CD3/TCR complex and/or an agent that stimulates a costimulatory molecule on the surface of the cells, or if contacted, the contacting step is less than 2 hours, for example, no more than 1 or 1.5 hours. In some embodiments, the agent that stimulates a CD3/TCR complex is an agent that stimulates CD3 (for example, an anti-CD3 antibody). In some embodiments, the agent that stimulates a costimulatory molecule is an agent that stimulates CD28, ICOS, CD27, HVEM, LIGHT, CD40, 4-1BB, OX40, DR3, GITR, CD30, TIM1, CD2, CD226, or any combination thereof. In some embodiments, the agent that stimulates a costimulatory molecule is an agent that stimulates CD28. In some embodiments, the agent that stimulates a CD3/TCR complex or the agent that stimulates a costimulatory molecule is chosen from an antibody (for example, a single-domain antibody (for example, a heavy chain variable domain antibody), a peptibody, a Fab fragment, or a scFv), a small molecule, or a ligand (for example, a naturally-existing, recombinant, or chimeric ligand).

In some embodiments, steps (1) and/or (2) are performed in cell media comprising no more than 5, 4, 3, 2, 1, or 0% serum. In some embodiments, steps (1) and/or (2) are performed in cell media comprising no more than 2% serum. In some embodiments, steps (1) and/or (2) are performed in cell media comprising about 2% serum. In some embodiments, steps (1) and/or (2) are performed in cell media comprising a LSD1 inhibitor or a MALT1 inhibitor. In some embodiments, step (1) is performed in cell media comprising no more than 5, 4, 3, 2, 1, or 0% serum. In some embodiments, step (1) is performed in cell media comprising no more than 2% serum. In some embodiments, step (1) is performed in cell media comprising about 2% serum. In some embodiments, step (2) is performed in cell media comprising no more than 5, 4, 3, 2, 1, or 0% serum. In some embodiments, step (2) is performed in cell media comprising no more than 2% serum. In some embodiments, step (2) is performed in cell media comprising about 2% serum. In some embodiments, step (1) is performed in cell media comprising a LSD1 inhibitor or a MALT1 inhibitor. In some embodiments, step (2) is performed in cell media comprising a LSD1 inhibitor or a MALT1 inhibitor.

In some embodiments, the aforementioned methods further comprise prior to step (i): (iv) receiving a fresh leukapheresis product (or an alternative source of hematopoietic tissue such as a fresh whole blood product, a fresh bone marrow product, or a fresh tumor or organ biopsy or removal (for example, a fresh product from thymectomy)) from an entity, for example, a laboratory, hospital, or healthcare provider.

In some embodiments, the aforementioned methods further comprise prior to step (i): (v) isolating the population of cells (for example, T cells, for example, CD8+ and/or CD4+ T cells) contacted in step (i) from a fresh leukapheresis product (or an alternative source of hematopoietic tissue such as a fresh whole blood product, a fresh bone marrow product, or a fresh tumor or organ biopsy or removal (for example, a fresh product from thymectomy)). In some embodiments, step (iii) is performed no later than 35 hours after the beginning of step (v), for example, no later than 27, 28, 29, 30, 31, 32, 33, 34, or 35 hours after the beginning of step (v), for example, no later than 30 hours after the beginning of step (v). In some embodiments, the population of cells from step (iii) are not expanded, or expanded by no more than 5, 10, 15, 20, 25, 30, 35, or 40%, for example, no more than 10%, for example, as assessed by the number of living cells, compared to the population of cells at the end of step (v).

In some embodiments, the aforementioned methods further comprise prior to step (i): receiving cryopreserved T cells isolated from a leukapheresis product (or an alternative source of hematopoietic tissue such as cryopreserved T cells isolated from whole blood, bone marrow, or tumor or organ biopsy or removal (for example, thymectomy)) from an entity, for example, a laboratory, hospital, or healthcare provider.

In some embodiments, the aforementioned methods further comprise prior to step (i): (iv) receiving a cryopreserved leukapheresis product (or an alternative source of hematopoietic tissue such as a cryopreserved whole blood product, a cryopreserved bone marrow product, or a cryopreserved tumor or organ biopsy or removal (for example, a cryopreserved product from thymectomy)) from an entity, for example, a laboratory, hospital, or healthcare provider.

In some embodiments, the aforementioned methods further comprise prior to step (i): (v) isolating the population of cells (for example, T cells, for example, CD8+ and/or CD4+ T cells) contacted in step (i) from a cryopreserved leukapheresis product (or an alternative source of hematopoietic tissue such as a cryopreserved whole blood product, a cryopreserved bone marrow product, or a cryopreserved tumor or organ biopsy or removal (for example, a cryopreserved product from thymectomy)). In some embodiments, step (iii) is performed no later than 35 hours after the beginning of step (v), for example, no later than 27, 28, 29, 30, 31, 32, 33, 34, or 35 hours after the beginning of step (v), for example, no later than 30 hours after the beginning of step (v). In some embodiments, the population of cells from step (iii) are not expanded, or expanded by no more than 5, 10, 15, 20, 25, 30, 35, or 40%, for example, no more than 10%, for example, as assessed by the number of living cells, compared to the population of cells at the end of step (v).

In some embodiments, the population of cells at the beginning of step (i) or step (1) has been enriched for IL6R-expressing cells (for example, cells that are positive for IL6Rα and/or IL6Rβ). In some embodiments, the population of cells at the beginning of step (i) or step (1) comprises no less than 40, 45, 50, 55, 60, 65, or 70% of IL6R-expressing cells (for example, cells that are positive for IL6Rα and/or IL6Rβ).

In some embodiments, steps (i) and (ii) or steps (1) and (2) are performed in cell media comprising IL-15 (for example, hetIL-15 (IL15/sIL-15Ra)). In some embodiments, IL-15 increases the ability of the population of cells to expand, for example, 10, 15, 20, or 25 days later. In some embodiments, IL-15 increases the percentage of IL6Rβ-expressing cells in the population of cells.

In some embodiments of the aforementioned methods, the methods are performed in a closed system. In some embodiments, T cell separation, activation, transduction, incubation, and washing are all performed in a closed system. In some embodiments of the aforementioned methods, the methods are performed in separate devices. In some embodiments, T cell separation, activation and transduction, incubation, and washing are performed in separate devices.

In some embodiments of the aforementioned methods, the methods further comprise adding an adjuvant or a transduction enhancement reagent in the cell culture medium to enhance transduction efficiency. In some embodiments, the adjuvant or transduction enhancement reagent comprises a cationic polymer. In some embodiments, the adjuvant or transduction enhancement reagent is chosen from: LentiBOOST™ (Sirion Biotech), vectofusin-1, F108, hexadimethrine bromide (Polybrene), PEA, Pluronic F68, Pluronic F127, Synperonic or LentiTrans™. In some embodiments, the adjuvant is LentiBOOST™ (Sirion Biotech).

In some embodiments of the aforementioned methods, the transducing the population of cells (for example, T cells) with a viral vector comprises subjecting the population of cells and viral vector to a centrifugal force under conditions such that transduction efficiency is enhanced. In an embodiment, the cells are transduced by spinoculation.

In some embodiments of the aforementioned methods, cells (e.g., T cells) are activated and transduced in a cell culture flask comprising a gas-permeable membrane at the base that supports large media volumes without substantially compromising gas exchange. In some embodiments, cell growth is achieved by providing access, e.g., substantially uninterrupted access, to nutrients through convection.

In some embodiments of the aforementioned methods, the CAR or CCAR comprises an antigen binding domain, a transmembrane domain, and an intracellular signaling domain.

In some embodiments, the antigen binding domain binds to an antigen chosen from: CD19, CD20, CD22, BCMA, mesothelin, EGFRvIII, GD2, Tn antigen, sTn antigen, Tn-O-Glycopeptides, sTn-O-Glycopeptides, PSMA, CD97, TAG72, CD44v6, CEA, EPCAM, KIT, IL-13Ra2, leguman, GD3, CD171, IL-11Ra, PSCA, MAD-CT-1, MAD-CT-2, VEGFR2, LewisY, CD24, PDGFR-beta, SSEA-4, folate receptor alpha, ERBBs (for example, ERBB2), Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, NY-ESO-1, GPR20, Ly6k, OR51E2, TARP, GFRα4, or a peptide of any of these antigens presented on MHC. In some embodiments, the antigen binding domain comprises a CDR, VH, VL, scFv or a CAR sequence disclosed herein. In some embodiments, the antigen binding domain comprises a VH and a VL, wherein the VH and VL are connected by a linker, optionally wherein the linker comprises the amino acid sequence of SEQ ID NO: 63 or 104.

In some embodiments, the transmembrane domain comprises a transmembrane domain of a protein chosen from the alpha, beta or zeta chain of T-cell receptor, CD28, CD3 epsilon, CD45, CD4, CD5, CD8, CD9, CD16, CD22, CD33, CD37, CD64, CD80, CD86, CD134, CD137 and CD154. In some embodiments, the transmembrane domain comprises a transmembrane domain of CD8. In some embodiments, the transmembrane domain comprises the amino acid sequence of SEQ ID NO: 6, or an amino acid sequence having at least about 85%, 90%, 95%, or 99% sequence identity thereof. In some embodiments, the nucleic acid molecule comprises a nucleic acid sequence encoding the transmembrane domain, wherein the nucleic acid sequence comprises the nucleic acid sequence of SEQ ID NO: 17, or a nucleic acid sequence having at least about 85%, 90%, 95%, or 99% sequence identity thereof.

In some embodiments, the antigen binding domain is connected to the transmembrane domain by a hinge region. In some embodiments, the hinge region comprises the amino acid sequence of SEQ ID NO: 2, 3, or 4, or an amino acid sequence having at least about 85%, 90%, 95%, or 99% sequence identity thereof. In some embodiments, the nucleic acid molecule comprises a nucleic acid sequence encoding the hinge region, wherein the nucleic acid sequence comprises the nucleic acid sequence of SEQ ID NO: 13, 14, or 15, or a nucleic acid sequence having at least about 85%, 90%, 95%, or 99% sequence identity thereof.

In some embodiments, the intracellular signaling domain comprises a primary signaling domain. In some embodiments, the primary signaling domain comprises a functional signaling domain derived from CD3 zeta, TCR zeta, FcR gamma, FcR beta, CD3 gamma, CD3 delta, CD3 epsilon, CD5, CD22, CD79a, CD79b, CD278 (ICOS), FcεRI, DAP10, DAP12, or CD66d. In some embodiments, the primary signaling domain comprises a functional signaling domain derived from CD3 zeta. In some embodiments, the primary signaling domain comprises the amino acid sequence of SEQ ID NO: 9 or 10, or an amino acid sequence having at least about 85%, 90%, 95%, or 99% sequence identity thereof. In some embodiments, the nucleic acid molecule comprises a nucleic acid sequence encoding the primary signaling domain, wherein the nucleic acid sequence comprises the nucleic acid sequence of SEQ ID NO: 20 or 21, or a nucleic acid sequence having at least about 85%, 90%, 95%, or 99% sequence identity thereof.

In some embodiments, the intracellular signaling domain comprises a costimulatory signaling domain. In some embodiments, the costimulatory signaling domain comprises a functional signaling domain derived from a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CD5, ICAM-1, 4-1BB (CD137), B7-H3, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD28-OX40, CD28-4-1BB, or a ligand that specifically binds with CD83. In some embodiments, the costimulatory signaling domain comprises a functional signaling domain derived from 4-1BB. In some embodiments, the costimulatory signaling domain comprises the amino acid sequence of SEQ ID NO: 7, or an amino acid sequence having at least about 85%, 90%, 95%, or 99% sequence identity thereof. In some embodiments, the nucleic acid molecule comprises a nucleic acid sequence encoding the costimulatory signaling domain, wherein the nucleic acid sequence comprises the nucleic acid sequence of SEQ ID NO: 18, or a nucleic acid sequence having at least about 85%, 90%, 95%, or 99% sequence identity thereof.

In some embodiments, the intracellular signaling domain comprises a functional signaling domain derived from 4-1BB and a functional signaling domain derived from CD3 zeta. In some embodiments, the intracellular signaling domain comprises the amino acid sequence of SEQ ID NO: 7 (or an amino acid sequence having at least about 85%, 90%, 95%, or 99% sequence identity thereof) and the amino acid sequence of SEQ ID NO: 9 or 10 (or an amino acid sequence having at least about 85%, 90%, 95%, or 99% sequence identity thereof). In some embodiments, the intracellular signaling domain comprises the amino acid sequence of SEQ ID NO: 7 and the amino acid sequence of SEQ ID NO: 9 or 10.

In some embodiments, the CAR or CCAR further comprises a leader sequence comprising the amino acid sequence of SEQ ID NO: 1.

In some embodiments, this disclosure features a population of CAR-expressing cells (for example, CCAR-expressing cells) (for example, autologous or allogeneic CAR-expressing T cells or NK cells) made by any of the aforementioned methods or any other method disclosed herein. In some embodiments, disclosed herein is a pharmaceutical composition comprising a population of CAR-expressing cells disclosed herein and a pharmaceutically acceptable carrier.

In some embodiments, in the final CAR cell product manufactured using the methods described herein, the total amount of beads (e.g., CD4 beads, CD8 beads, and/or TransACT beads) is no more than 0.04, 0.05, 0.06, 0.07, 0.08, 0.09, 0.1, 0.2, 0.3, 0.4, or 0.5% of the total amount of beads added during the manufacturing process.

In some embodiments, this disclosure features a population of CAR-expressing cells (for example, CCAR-expressing cells) (for example, autologous or allogeneic CAR-expressing T cells or NK cells) comprising one or more of the following characteristics: (a) about the same percentage of naïve cells, for example, naïve T cells, for example, CD45RO− CCR7+ T cells, as compared to the percentage of naïve cells, for example, naïve T cells, for example, CD45RO− CCR7+ cells, in the same population of cells prior to being engineered to express the CAR; (b) a change within about 5% to about 10% of naïve cells, for example, naïve T cells, for example, CD45RO− CCR7+ T cells, for example, as compared to the percentage of naïve cells, for example, naïve T cells, for example, CD45RO− CCR7+ cells, in the same population of cells prior to being engineered to express the CAR; (c) an increased percentage of naïve cells, for example, naïve T cells, for example, CD45RO− CCR7+ T cells, for example, increased by at least 1.2, 1.4, 1.6, 1.8, 2.0, 2.2, 2.4, 2.6, 2.8, or 3-fold, as compared to the percentage of naïve cells, for example, naïve T cells, for example, CD45RO− CCR7+ cells, in the same population of cells prior to being engineered to express the CAR; (d) about the same percentage of central memory cells, for example, central memory T cells, for example, CCR7+CD45RO+ T cells, as compared to the percentage of central memory cells, for example, central memory T cells, for example, CCR7+CD45RO+ T cells, in the same population of cells prior to being engineered to express the CAR; (e) a change within about 5% to about 10% of central memory cells, for example, central memory T cells, for example, CCR7+CD45RO+ T cells, as compared to the percentage of central memory cells, for example, central memory T cells, for example, CCR7+CD45RO+ T cells, in the same population of cells prior to being engineered to express the CAR; (f) a decreased percentage of central memory cells, for example, central memory T cells, for example, CCR7+CD45RO+ T cells, for example, decreased by at least 20, 25, 30, 35, 40, 45, or 50%, as compared to the percentage of central memory cells, for example, central memory T cells, for example, CCR7+CD45RO+ T cells, in the same population of cells prior to being engineered to express the CAR; (g) about the same percentage of stem memory T cells, for example, CD45RA+CD95+IL-2 receptor β+CCR7+CD62L+ T cells, as compared to the percentage of stem memory T cells, for example, CD45RA+CD95+IL-2 receptor β+CCR7+CD62L+ T cells, in the same population of cells prior to being engineered to express the CAR; (h) a change within about 5% to about 10% of stem memory T cells, for example, CD45RA+CD95+IL-2 receptor β+CCR7+CD62L+ T cells, as compared to the percentage of stem memory T cells, for example, CD45RA+CD95+IL-2 receptor β+CCR7+CD62L+ T cells, in the same population of cells prior to being engineered to express the CAR; or (i) an increased percentage of stem memory T cells, for example, CD45RA+CD95+IL-2 receptor β+CCR7+CD62L+ T cells, as compared to the percentage of stem memory T cells, for example, CD45RA+CD95+IL-2 receptor β+CCR7+CD62L+ T cells, in the same population of cells prior to being engineered to express the CAR.

In some embodiments, this disclosure features a population of CAR-expressing cells (for example, CCAR-expressing cells) (for example, autologous or allogeneic CAR-expressing T cells or NK cells), wherein: (a) the median GeneSetScore (Up TEM vs. Down TSCM) of the population of cells is about the same as or differs by no more than (for example, increased by no more than) about 25, 50, 75, 100, or 125% from the median GeneSetScore (Up TEM vs. Down TSCM) of the same population of cells prior to being engineered to express the CAR; (b) the median GeneSetScore (Up Treg vs. Down Teff) of the population of cells is about the same as or differs by no more than (for example, increased by no more than) about 25, 50, 100, 150, or 200% from the median GeneSetScore (Up Treg vs. Down Teff) of the population of cells prior to being engineered to express the CAR; (c) the median Gene SetScore (Down stemness) of the population of cells is about the same as or differs by no more than (for example, increased by no more than) about 25, 50, 100, 150, 200, or 250% from the median GeneSetScore (Down stemness) of the population of cells prior to being engineered to express the CAR; (d) the median GeneSetScore (Up hypoxia) of the population of cells is about the same as or differs by no more than (for example, increased by no more than) about 125, 150, 175, or 200% from the median GeneSetScore (Up hypoxia) of the population of cells prior to being engineered to express the CAR; or (e) the median GeneSetScore (Up autophagy) of the population of cells is about the same as or differs by no more than (for example, increased by no more than) about 180, 190, 200, or 210% from the median GeneSetScore (Up autophagy) of the population of cells prior to being engineered to express the CAR.

In some embodiments, this disclosure features a method of increasing an immune response in a subject, comprising administering a population of CAR-expressing cells disclosed herein or a pharmaceutical composition disclosed herein to the subject, thereby increasing an immune response in the subject.

In some embodiments, disclosed herein is a method of treating a cancer in a subject, comprising administering a population of CAR-expressing cells disclosed herein or a pharmaceutical composition disclosed herein to the subject, thereby treating the cancer in the subject. In some embodiments, the cancer is a solid cancer, for example, chosen from: one or more of mesothelioma, malignant pleural mesothelioma, non-small cell lung cancer, small cell lung cancer, squamous cell lung cancer, large cell lung cancer, pancreatic cancer, pancreatic ductal adenocarcinoma, esophageal adenocarcinoma , breast cancer, glioblastoma, ovarian cancer, colorectal cancer, prostate cancer, cervical cancer, skin cancer, melanoma, renal cancer, liver cancer, brain cancer, thymoma, sarcoma, carcinoma, uterine cancer, kidney cancer, gastrointestinal cancer, urothelial cancer, pharynx cancer, head and neck cancer, rectal cancer, esophagus cancer, or bladder cancer, or a metastasis thereof. In some embodiments, the cancer is a liquid cancer, for example, chosen from: chronic lymphocytic leukemia (CLL), mantle cell lymphoma (MCL), multiple myeloma, acute lymphoid leukemia (ALL), Hodgkin lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitts lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+ large B-cell lymphoma, large B-cell lymphoma arising in HHV8-associated multicentric Castleman disease, primary effusion lymphoma, B-cell lymphoma, acute myeloid leukemia (AML), or unclassifiable lymphoma.

In some embodiments, the method further comprises administering a second therapeutic agent to the subject. In some embodiments, the second therapeutic agent is an anti-cancer therapeutic agent, for example, a chemotherapy, a radiation therapy, or an immune-regulatory therapy. In some embodiments, the second therapeutic agent is IL-15 (for example, hetIL-15 (IL15/sIL-15Ra)).

Although methods and materials similar or equivalent to those described herein can be used in the practice or testing of the present disclosure, suitable methods and materials are described below. All publications, patent applications, patents, and other references (for example, sequence database reference numbers) mentioned herein are incorporated by reference in their entirety. For example, all GenBank, Unigene, and Entrez sequences referred to herein, for example, in any Table herein, are incorporated by reference. When one gene or protein references a plurality of sequence accession numbers, all of the sequence variants are encompassed.

In addition, the materials, methods, and examples are illustrative only and not intended to be limiting. Headings, sub-headings or numbered or lettered elements, for example, (a), (b), (i) etc., are presented merely for ease of reading. The use of headings or numbered or lettered elements in this document does not require the steps or elements be performed in alphabetical order or that the steps or elements are necessarily discrete from one another. Other features, objects, and advantages of this disclosure will be apparent from the description and drawings, and from the claims.

### BRIEF DESCRIPTION OF THE FIGURES

FIGS. 1A-1I: When purified T cells were incubated with cytokines, the naïve cells were the predominant population transduced. FIG. A is a graph showing exemplary cytokine process. FIG. 1B is a pair of graphs showing the percentages of CD3+ CAR+ cells at each indicated time point after transduction. FIG. 1C is a set of graphs showing the transduction within the CD3+CCR7+CD45RO− population in a CD3/CD28 bead stimulated populations (left) compared to cytokines only populations (right) in two independent donors. For the sample referred to as “Short stim IL7+IL15” in FIG. 1C, the cells were stimulated with beads for 2 days and then they were removed in the presence of IL7 and IL15. FIGS. 1D, 1E, and 1F are a set of flow cytometry graphs showing the transduction of T-cell subsets cultured with IL2 (FIG. 1D), IL15 (FIG. 1E), and IL7+IL15 (FIG. 1F) daily over a three-day period. FIG. 1G is a set of flow cytometry graphs showing the T cell differentiation on day 0 (left) and on day 1 (right) for CCR7 and CD45RO after stimulation with IL2 (upper right panel) or IL-15 (lower right panel). FIGS. 1H and 1I are a set of graphs showing the percentages of CD3+CCR7+RO−, CD3+CCR7+RO+, CD3+CCR7−RO+, and CD3+CCR7−RO− cells at day 0 or after 24-hour incubation with the indicated cytokines.

FIGS. 2A-2D: CARTs generated with one day of cytokine stimulation were functional. FIG. 2A: Purified T cells were transduced with a MOI of 1 and in all the cytokine conditions tested, the percentages of CAR-expressing cells observed at day 1 and day 10 were similar. The CARTs were generated within one day and expanded via CD3/CD28 beads after harvest for 9 days to mimic the in vivo setting. FIG. 2A is a pair of graphs showing the average percentages of CD3+ CAR+ cells under each condition for day 1 CARTs (left) and day 10 CARTs (right). FIG. 2B: The cytotoxicity capacity of the day 1 CARTs post expansion was measured using Nalm6 as the target cells. FIG. 2B is a graph showing % killing of CD19 positive Nalm6 cells by CARTs from each condition. Day 10 CARTs expanded using CD3/CD28 beads are marked as “Day 10.” All the other samples were day 1 CARTs. FIG. 2C: The secretion of IFNg of the expanded day 1 CARTs in response to Nalm6 target cells was tested. FIG. 2C is a graph showing the amount of IFN-gamma secretion by CARTs from each condition in the presence of CD19 positive or CD19 negative target cells. FIG. 2D: The proliferative capacity of the day 1 CARTs was tested by measurement of the incorporation of EDU. FIG. 2D is a graph showing the average percentages of EDU-positive cells for each condition. Similar to FIG. 2B, day 10 CARTs are marked as “Day 10” and all the other samples were day 1 CARTs.

FIGS. 3A-3B: The impact of MOI and media composition on transduction on day 0. FIG. 3A: Purified T cells were transduced with a range of MOIs from 1 to 10 in the presence of IL15, IL2+IL15, IL2+IL7, or IL7+IL15. Regardless of cytokine used, a linear increase in transduction was observed. FIG. 3A is a set of graphs where the percentages of CD3+ CAR+ cells are plotted against MOIs for each condition tested. FIG. 3B: The composition of the media impacted the transduction in the cytokine process. FIG. 3B is a pair of graphs showing the percentages of CD3+ CAR+ cells on day 1 (left) or day 8 (right) for each condition tested. “2.50” indicates a MOI of 2.50. “5.00” indicates a MOI of 5.00.

FIGS. 4A-4D: CAR T cells generated within 24 hours can eliminate tumor. FIG. 4A: Purified T cells were transduced with CAR19 and 24 hours later were harvested. FIG. 4A is a set of flow cytometry plots showing the transduction of T cells with CAR19 that were cultured with IL2, IL15 and IL7+IL15, illustrating the transduction with each cytokine condition. FIG. 4B: A graph showing average viability which was above 80% in all the conditions tested. FIG. 4C: The expansion of the day 1 CARTs in the peripheral blood is increased in vivo as compared to their day 10 counterparts. The percentage of live CD45+CD11b−CD3+CAR+ cells at indicated time points after infusion for each condition tested. The day 10 CARTs are marked as “D10 1e6” or “D10 5e6” and all the other samples were day 1 CARTs. FIG. 4D: The day 1 CARTs could eliminate tumor in vivo although with a delayed kinetics as compared to the day 10 CARTs. FIG. 4D is a graph showing total flux at indicated time points after tumor inoculation for each condition tested. CARTs were administered 4 days after tumor inoculation. The day 10 CARTs are marked as “5e6 d. 10” and all the other samples were day 1 CARTs.

FIGS. 5A-5B: The cytokine process was scalable. FIG. 5A: The T cells were enriched on a CliniMACS® Prodigy® and the B cell compartment was reduced to less than 1%. FIG. 5A is a set of flow cytometry plots showing the staining of cells with an anti-CD3 antibody (left) or an anti-CD19 antibody and an anti-CD14 antibody (right) for leukopak cells (upper) or cells post CD4+CD8+ enrichment (lower). FIG. 5B: Purified T cells from a frozen apheresis were transduced with CAR19 in either a 24 well plate or a PL30 bag post enrichment. The CARTs were harvested 24 hours later. FIG. 5B is a set of flow cytometry plots showing staining for CD3 and CAR of cells manufactured in the presence of either IL2 or hetIL-15 (IL15/sIL-15Ra).

FIGS. 6A-6C: The CARTs manufactured by the activation process showed superior anti-tumor efficacy in vivo. FIGS. 6A and 6B are graphs where tumor burden is plotted against the indicated time point after tumor implantation. “d.1” indicates CARTs manufactured using the activation process. “d.9” indicates CARTs manufactured with a traditional 9-day expansion protocol, serving as a positive control in this study. FIG. 6C is a set of representative images showing bioluminescence from mice.

FIGS. 7A-7B: IL6Rα and IL6Rβ expressing cells were enriched in less differentiated T cell population. Fresh T cells were stained for indicated surface antigens and examined for expression levels of IL6Rα and IL6Rβ on CD4 (FIG. 7A) and CD8 (FIG. 7B) T cell subsets.

FIGS. 8A and 8B: Both IL6Rα and IL6Rβ expressing cells were enriched in less differentiated T cell population. Fresh T cells were stained for indicated surface antigens and examined for expression levels of indicated surface antigens on CD4 (FIG. 8A) and CD8 (FIG. 8B) T cell subsets.

FIG. 9: IL6Rα expressing cells expressed surface markers of less differentiated T cells. Fresh T cells were stained for indicated surface antigens and examined for expression levels of various surface antigens in IL6Rα high, middle, and low expressing cell subsets.

FIG. 10: IL6Rβ expressing cells expressed surface markers of less differentiated T cells. Fresh T cells were stained for indicated surface antigens and examined for expression levels of various surface antigens in IL6Rβ high, middle, and low expressing cell subsets.

FIG. 11: IL6Rα but not IL6Rβ expression was down-regulated following TCR engagement. T cells were activated with αCD3αCD28 beads at day 0 and then examined for expression levels of IL6Rα and IL6Rβ at indicated time points.

FIG. 12: Fold expansion of cytokine treated T cells after TCR engagement. T cells were activated with αCD3αCD28 beads at day 0 in the presence of indicated cytokines and then monitored for cell numbers at indicated time points.

FIGS. 13A and 13B: IL2, IL7, and IL15 treatment did not affect cell size and viability after TCR engagement. T cells were activated with αCD3αCD28 beads at day 0 in the presence of indicated cytokines and then monitored for cell size (FIG. 13A) and viability (FIG. 13B) at indicated time points.

FIG. 14: Expression kinetics of various surface molecules on CD4 T cells after cytokine treatment. T cells were activated with αCD3αCD28 beads at day 0 in the presence of indicated cytokines and then examined for expression of various surface molecules by flow cytometry at indicated time points.

FIG. 15: Expression kinetics of various surface molecules on CD8 T cells after cytokine treatment. T cells were activated with αCD3αCD28 beads at day 0 in the presence of indicated cytokines and then examined for expression of various surface molecules by flow cytometry at indicated time points.

FIG. 16: IL6Rβ expression was mainly restricted on CD27 expressing T cell subsets after TCR engagement. T cells were activated with αCD3αCD28 beads at day 0 in the presence of indicated cytokines and then examined for IL6Rβ expression by flow cytometry at day 15.

FIG. 17: IL6Rβ expression was mainly restricted on CD57 non-expressing T cell subsets after TCR engagement. T cells were activated with αCD3αCD28 beads at day 0 in the presence of indicated cytokines and then examined for IL6Rβ expression by flow cytometry at day 25.

FIG. 18: Common γ-chain cytokine treated T cells produced functional cytokines at day 25. T cells were activated with αCD3αCD28 beads at day 0 in the presence of indicated cytokines and then examined for percentages of IL2, IFNγ, and TNFα producing T cells by flow cytometry at day 25.

FIGS. 19A and 19B: BCMA CAR expression on Day 1 using ARM at MOI=2.5 in T cells from two healthy donors. FIG. 19A is a panel of histograms showing BCMA CAR expression as measured by flow cytometry. FIG. 19B is a table listing reagents/conditions used in the flow cytometry analysis.

FIGS. 20A, 20B, and 20C: In vitro CAR expression kinetics from day 1 to day 4 of cells manufactured using the ARM process. CARs were stably expressed on day 3. FIG. 20A is a panel of histograms showing CAR expression at the indicated time points measured by flow cytometry. FIGS. 20B and 20C are graphs showing CAR+% and MFI values over time, respectively.

FIGS. 21A and 21B: In vivo triage in a KMS-11-luc multiple myeloma xenograft mouse model. Each mouse received 1.5E6 of day 1 CART product. FIG. 21A is a panel of histograms showing the day 1 and day 7 CAR expression in the CART cells. FIG. 21B is a graph showing the tumor kinetics (BLI level) after CART treatment.

FIGS. 22A, 22B, and 22C: In vivo triage of BCMA CAR using dose titration in a KMS-11-luc multiple myeloma xenograft mouse model. FIG. 22A is a panel of histograms showing the CAR expression at day 1 and day 3. FIG. 22B is a graph showing tumor intake kinetics after CART treatment using two different doses: a dose of 1.5e5 CAR+ T cells and a dose of 5e4 CAR+ T cells. The doses of CAR+ cells were normalized based on the day 3 CAR expression. FIG. 22C is a graph showing body weight kinetics over the course of this study.

FIGS. 23A, 23B, and 23C. FIGS. 23A and 23B are graphs showing percentage of T cell expressing the CAR on their cell surface (FIG. 23A) and mean fluorescence intensity (MFI) of CD3+CAR+ cells (FIG. 23B) observed over time (replicate efficiencies are averaged from the two flow panels shown in FIG. 23C). FIG. 23C is a panel of flow cytometry plots showing gating strategy for surface CAR expression on viable CD3+ cells, as based on UTD samples. Numbers in the plots indicate percent CAR positive.

FIGS. 24A and 24B. FIG. 24A is a graph showing end-to-end composition of the starting material (Prodigy® product) and at harvest at various time points after culture initiation. Naive (n), central memory (cm), effector memory (em), and effector (eff) subsets were defined by CD4, CD8, CCR7, and CD45RO surface expression or lack thereof. CD4 composition is indicated. For each time point, the left bar shows cell composition of the overall CD3+ population (bulk) and the right bar shows cell composition of the CAR+ fraction. FIG. 24B is a panel of flow cytometry plots showing gating strategy applied on live CD3+ events to determine overall transduction efficiency (top row), CD4/CD8 composition (middle row), and memory subsets (bottom row) within the overall CD3+ population (bulk) and the CAR+ fraction.

FIG. 25. Kinetics of T cell subsets expressing surface CAR over time, expressed as number of viable cells in the respective subsets.

FIG. 26. Viable cell recovery (number of viable cells recovered at harvest versus number of viable cells seeded) 12 to 24 hours after culture initiation as determined from pre-wash counts.

FIG. 27. Viability of rapid CARTs harvested 12 to 24 hours after culture initiation, as determined pre-wash and post-wash at the time of harvest.

FIGS. 28A, 28B, 28C, and 28D. FIG. 28A is a graph showing composition of the starting material (healthy donor leukopak; LKPK) and the T cell-enriched product as analyzed by flow cytometry. Numbers indicate % of parent (live, single cells). T: T cells; mono: monocytes; B: B cells; CD56 (NK): NK cells. FIG. 28B is a panel of flow cytometry plots showing gating strategy on live CD3+ events used to determine transduction rate (forward scatter FSC vs. CAR) and T cell subsets (CD4 vs. CD8 and CCR7 vs. CD45RO). For ARM-CD19 CAR (CD19 CART cells manufactured using the Activated Rapid Manufacturing (ARM) process) and TM-CD19 CAR (CD19 CART cells manufactured using the traditional manufacturing (TM) process), the left lower panels represent bulk cultures, while the right panels represent CAR+ T cells. “ARM-UTD” and “TM-UTD” refer to untransduced T cells (UTD) manufactured according to the ARM and the TM processes, respectively. Numbers in quadrants indicate % of parental population. Boxes in the TM-UTD and TM-CD19 CAR plots indicate skewing toward a T_{CM} phenotype for the TM process. Boxes in the ARM-UTD and ARM-CD19 CAR plots indicate the maintenance of naïve-like cells by the ARM process. NA: not applicable. FIG. 28C is a graph showing end-to-end T cell composition of ARM-CD19 CAR and TM-CD19 CAR. Composition is shown for “bulk” and “CAR+” populations where applicable. The percentage of the respective populations refers to % of parental, either CD3+ or CAR+CD3+ as applicable. The % of CD4 cells of the respective bulk or CAR+ population is indicated. LKPK: Leukopak starting material; 4 and 8: CD4+ and CD8+, respectively; eff: effector; em: effector memory; cm: central memory; n: naïve-like. Data is representative of 3 full-scale runs with 3 different healthy donors (n=3) and several small-scale runs used to optimize the process. FIG. 28D is a table showing the percentages shown in FIG. 28C.

FIGS. 29A, 29B, 29C, and 29D. Cytokine concentration in cell culture supernatants. IFN-γ (FIGS. 29A and 29B) and IL-2 (FIGS. 29C and 29D). FIGS. 29A and 29C: TM-CD19 CAR, ARM-CD19 CAR, and respective UTD were co-cultured with NALM6-WT (ALL), TMD-8 (DLBCL), or without cancer cells (T cells alone). Supernatant was collected 48 h later. FIGS. 29B and 29D: ARM-CD19 CAR was cocultured with NALM6-WT, NALM6-19KO (CD19-negative) or alone. Supernatant was collected after 24 h or 48 h. To further assess antigen-specific cytokine secretion, ARM-CD19 CAR was cultured alone for 24 h, washed and then co-cultured with target cells for 24 h. Data shown is derived from 2 healthy donor T cells and is representative of 2 experiments with three donors total.

FIGS. 30A, 30B, and 30C. FIG. 30A is a graph outlining the xenograft mouse model to study the anti-tumor activity of ARM-CD19 CAR. FIG. 30B is a panel of flow cytometry plots showing determination of CAR expression on ARM-CD19 CAR cells from a sentinel vial. ARM-CD19 CAR cells were cultured for the time period described in the figure, prior to flow-cytometry analysis. Gating for CAR expression was based on an isotype control (Iso) staining. FIG. 30C is a graph showing in vivo efficacy of ARM-CD19 CAR in the xenograft mouse model. NSG mice were injected with the pre-B ALL line NALM6, expressing the luciferase reporter gene; the tumor burden is expressed as total body luminescence (p/s), depicted as mean tumor burden with 95% confidence interval. On day 7 post tumor inoculation, mice were treated with ARM-CD19 CAR or TM-CD19 CAR at the respective doses (number of viable CAR+ T cells). High dose ARM-CD19 CAR group was terminated on day 33 due to onset of X-GVHD. Vehicle (PBS) and non-transduced T cells (UTD) served as negative controls. n=5 mice for all groups, except n=4 for ARM-UTD 1×10⁶ dose and all TM-CD19 CAR dose groups. Five xenograft studies were run with CAR-T cells generated from 5 different healthy donors, three of which included a comparison to TM-CD19 CAR.

FIGS. 31A, 31B, 31C, and 31D. Plasma cytokine levels of NALM6 tumor-bearing mice treated with ARM-CD19 CAR or TM-CD19 CAR at respective CAR-T cell doses. Mice were bled and plasma cytokine measured by MSD assay. IFN-γ (FIGS. 31A and 31B) and IL-2 (FIGS. 31C and 31D) are shown for mice treated with CAR-T (FIGS. 31A and 31C) or ARM- and TM-UTD cells (FIGS. 31B and 31D). Bars within each dose represent the mean cytokine level within the group at different time points (from left: day 4, 7, 10, 12, 16, 19, 23, 26). Horizontal bars and numbers indicate the fold-change comparisons between ARM-CD19 CAR (1×10⁶ dose group) and TM-CD19 CAR (0.5×10⁶ dose group) described in the text: 3-fold for IFN-γ; and 10-fold for IL-2. Groups taken down due to tumor burden or body weight loss do not show the last time points. Plasma cytokine levels were measured for 2 studies. no tum: no tumor.

FIG. 32. Time course of total and CAR+ T cell concentrations in NALM6 tumor-bearing mice treated with PBS vehicle, UTD, TM-CD19 CAR, or ARM-CD19 CAR. Blood samples were taken at 4, 7, 14, 21 and 28 days post CAR-T cell injection. Total T cells (CD3+, upper) and CAR+ T cell (CD3+CAR+, lower) concentrations were analyzed by flow cytometry at designed time points, depicted as mean cells with 95% confidence interval.

FIGS. 33A and 33B. IL-6 protein levels in three-party co-culture supernatants in pg/mL. ARM-CD19 CAR/K562 co-cultured cells (FIG. 33A) or TM-CD19 CAR/K562 cell co-cultured cells (FIG. 33B), for 6 or 24 hours incubated at different ratios (1:1 and 1:2.5), were then added to PMA-differentiated THP-1 cells for another 24 hours. Results from CAR-T cells co-cultured with K562-CD19 cells, CAR-T cells co-cultured with K562-Mesothelin cells, and CAR-T cells alone are shown. 1:5 ratios are not shown for clarity. ARM-CD19 CAR only and TM-CD19 CAR only designated bars represent CAR-T cell cultures (6 h, 24 h) without target cells. Mean+SEM, duplicates of n=1 (TM-CD19 CAR) and n=3 (ARM-CD19 CAR).

FIGS. 34A, 34B, and 34C. ARM process preserves BCMA CAR+T cell stemness. PI61, RIGS and BCMA10 CART cells manufactured using the ARM process were assessed for CAR expression at thaw (FIG. 34A) and 48 h post-thaw (FIG. 34B). CCR7/CD45RO markers were also assessed for the 48 h post-thaw product (FIG. 34C). Data shown is one representative from two experiments performed using two donor T cells.

FIGS. 35A and 35B. The TM process mainly resulted in central-memory T cells (TCM) (CD45RO+/CCR7+), while the naïve-like T cell population is almost gone in the CAR+T cells with TM process. PI61, RIGS and BCMA10 CART cells manufactured using the TM process were assessed for CAR expression at day 9 (FIG. 35A). CCR7/CD45RO markers were also assessed at day 9 post-thaw product (FIG. 35B). Data shown is one representative from two experiments performed using two donor T cells.

FIGS. 36A, 36B, 36C, and 36D. ARM processed BCMA CAR-T cells demonstrates BCMA-specific activation and secretes higher levels of IL2 and IFN-γ. IL-2 and IFN-γ concentrations in cell culture supernatants. PI61, RIGS and BCMA10 CART cells manufactured using the ARM or TM process, and respective UTD were co-cultured with KMS-11 at 2.5:1 ratio. Supernatants were collected 20 h later. For the ARM products, IFN-γ concentrations are shown in FIG. 36A and IL-2 concentrations are shown in FIG. 36B. For the TM products, IFN-γ concentrations are shown in FIG. 36C and IL-2 concentrations are shown in FIG. 36D. Data shown is one representative from two experiments performed using two donor T cells.

FIGS. 37A, 37B, and 37C. Single cell RNA-seq data for input cells (FIG. 37A), Day 1 cells (FIG. 37B), and Day 9 cells (FIG. 37C). The “nGene” graphs show the number of expressed genes per cell. The “nUMI” graphs show the number of unique molecular identifiers (UMIs) per cell.

FIGS. 38A, 38B, 38C, and 38D. T-Distributed Stochastic Neighbor Embedding (TSNE) plots comparing input cells (FIG. 38A), Day 1 cells (FIG. 38B), and Day 9 cells (FIG. 38C) for a proliferation signature, which was determined based on expression of genes CCNB1, CCND1 , CCNE1, PLK1, and MKI67. Each dot represents a cell in that sample. Cells shown as light grey do not express the proliferation genes whereas dark shaded cells express one or more of the proliferation genes. FIG. 38D is a violin plot showing the distribution of gene set scores for a gene set comprised of genes that characterize a resting vs. activated T cell state for Day 1 cells, Day 9 cells, and input cells. In FIG. 38D, a higher gene set score (Up resting vs. Down activated) indicates an increasing resting T cell phenotype, whereas a lower gene set score (Up resting vs. Down activated) indicates an increasing activated T cell phenotype. Input cells were overall in more of a resting state compared to Day 9 and Day 1 cells. Day 1 cells show the greatest activation gene set score.

FIGS. 39A, 39B, 39C, 39D and 39E. Gene set analysis for input cells, Day 1 cells, and Day 9 cells. In FIG. 39A, a higher gene set score for the gene set “Up TEM vs. Down TSCM” indicates an increasing effector memory T cell (TEM) phenotype of the cells in that sample, whereas a lower gene set score indicates an increasing stem cell memory T cell (TSCM) phenotype. In FIG. 39B, a higher gene set score for the gene set “Up Treg vs. Down Teff” indicates an increasing regulatory T cell (Treg) phenotype, whereas a lower gene set score indicates an increasing effector T cell (Teff) phenotype. In FIG. 39C, a lower gene set score for the gene set “Down sternness” indicates an increasing sternness phenotype. In FIG. 39D, a higher gene set score for the gene set “Up hypoxia” indicates an increasing hypoxia phenotype. In FIG. 39E, a higher gene set score for the gene set “Up autophagy” indicates an increasing autophagy phenotype. Day 1 cells looked similar to the input cells in terms of memory, stem-like and differentiation signature. Day 9 cells, on the other hand, show a higher enrichment for metabolic stress.

FIGS. 40A, 40B, and 40C. Gene cluster analysis for input cells. FIGS. 40A-40C are violin plots showing the gene set scores from gene set analysis of the four clusters of the input cells. Each dot overlaying the violin plots in FIGS. 40A-40C represents a cell's gene set score. In FIG. 40A, a higher gene set score of the gene set “Up Treg vs. Down Teff” indicates an increasing Treg cell phenotype, whereas a lower gene set score of the gene set “Up Treg vs. Down Teff” indicates an increasing Teff cell phenotype. In FIG. 40B, a higher gene set score of the gene set “Progressively up in memory differentiation” indicates an increasing late memory T cell phenotype, whereas a lower gene set score of the gene set “Progressively up in memory differentiation” indicates an increasing early memory T cell phenotype. In FIG. 40C, a higher gene set score of the gene set “Up TEM vs. Down TN” indicates an increasing effector memory T cell phenotype, whereas a lower gene set score of the gene set “Up TEM vs. Down TN” indicates an increasing naïve T cell phenotype. The cells in Cluster 3 are shown to be in a later memory, further differentiated T cell state compared to the cells in Cluster 1 and Cluster 2 which are in an early memory, less differentiated T cell state. Cluster 0 appears to be in an intermediate T cell state. Taken together, this data shows that there is a considerable level of heterogeneity within input cells.

FIGS. 41A, 41B, and 41C. TCR sequencing and measuring clonotype diversity. Day 9 cells have flatter distribution of clonotype frequencies (higher diversity).

FIG. 42 is a flow chart showing the design of a Phase I clinical trial testing BCMA CART cells manufactured using the ARM process in adult patients with relapsed and/or refractory multiple myeloma.

FIG. 43 is a graph showing FACS analyses for ARM-BCMA CAR expression at different collection time points post viral addition in the presence or absence of AZT at two different concentrations (30 μM and 100 μM). Lentiviral vector was added lh later prior to AZT treatment at the time of activation and cell seeding.

FIGS. 44A and 44B are graphs showing assessment of ARM-BCMA CAR for CAR expression at thaw (FIG. 44A) and 48 h post-thaw and CCR7/CD45RO markers at 48 h post-thaw product as well as day 9 for TM-BCMA CAR (FIG. 44B). Data shown is one representative from two experiments performed using T cells from two donors.

FIGS. 45A and 45B are graphs showing cytokine concentrations in cell culture supernatants. ARM-BCMA CAR and TM-BCMA CAR, and respective UTD were co-cultured with KMS-11. Supernatant was collected 24 h later. Data shown is one representative from two experiments performed using T cells from two donors.

FIG. 46 is a graph showing outline of xenograft efficacy study to test ARM-BCMA.

FIG. 47 is a graph comparing the efficacy of ARM-BCMA CAR with that of TM-BCMA CAR in a xenograft model. NSG mice were injected with MM cell line KMS11, expressing the luciferase reporter gene. The tumor burden is expressed as total body luminescence (p/s), depicted as mean tumor burden +SEM. On day 8 post tumor inoculation, mice were treated with ARM-BCMA CAR or TM-BCMA CAR at the respective doses (number of viable CAR+ T cells). Vehicle (PBS) and UTD T cells served as negative controls. N=5 mice for all groups, except N=4 for ARM-BCMA CAR (1e4 cells), PBS, and UTD groups.

FIGS. 48A, 48B, and 48C are graphs showing plasma IFN-γ kinetics of mice treated with ARM-BCMA CAR or TM-BCMA CAR. Plasma IFN-γ levels of KMS11-luc tumor-bearing mice treated with UTD, ARM-BCMA CAR, or TM-BCMA CAR at respective CAR-T doses. All IFN-γ levels were depicted as mean±SEM. Mice were bled and plasma cytokine measured by Meso Scale Discovery (MSD) assay.

FIG. 49 is a graph showing cellular kinetics of ARM-BCMA CAR and TM-BCMA CAR in vivo. Cellular kinetics in peripheral blood of KMS11 tumor-bearing mice treated with TM UTD, ARM UTD, ARM-BCMA CAR, and TM-BCMA CAR at different doses. Cell count is expressed as mean cell count +SD. On day 8 post tumor inoculation, mice were treated with ARM-BCMA CAR or TM-BCMA CAR at the respective doses (number of viable CAR+ T cells). Vehicle (PBS) and UTD T cells served as negative controls. Blood samples were taken at 7, 14, and 21 days post CAR-T injection and were analyzed by flow cytometry at designed time points. N=5 mice for all groups, except N=4 for ARM-BCMA CAR (1e4 cells), PBS, and UTD groups.

FIGS. 50A and 50B are a pair of graphs showing percentage viability post 24 hours (FIG. 50A) and percentage recovery post 24 hours (FIG. 50B). The columns shown in FIGS. 50A and 50B represent data from, from left to right, CAR19 (MOI of 1), CAR19 (MOI of 2), CAR19.HilD (MOI of 1), CAR19.HilD (MOI of 2), UTD (MOI of 1), and UTD (MOI of 2).

FIGS. 51A-51D are graphs showing percent CAR expression in CAR19 cells (FIGS. 51A and 51B) or CAR19.HilD cells (FIGS. 51C and 51D) in the presence of lenalidomide or DMSO as indicated in the figures.

### DETAILED DESCRIPTION

### Definitions

Unless defined otherwise, all technical and scientific terms used herein have the same meaning as commonly understood by one of ordinary skill in the art to which this disclosure pertains.

“Controllable chimeric antigen receptor (CCAR),” as used herein, refers to a CAR, the level and/or activity of which can be regulated. In some embodiments, the CCAR's expression level or activity can be regulated to enhance CAR function and/or reduce toxicity. In some embodiments, the CCAR is regulated at a transcriptional, translational, or post-translational level. In some embodiments, the CCAR is regulated by an On switch that leads to the stabilization of the CAR or turns on the expression and/or activity of the CAR. In some embodiments, the CCAR is regulated by an Off switch that leads to the ubiquitination and degradation of the CAR or turns off the expression and/or activity of the CAR. In some embodiments, the CCAR is regulated by both an On switch and an Off switch. In some embodiments, the CCAR comprises a degron tag as disclosed in WO2019079569, herein incorporated by reference in its entirety. In some embodiments, the CCAR is a regulatable CAR (RCAR) disclosed in WO2015090229, herein incorporated by reference in its entirety. In some embodiments, the CCAR is a heterodimeric, conditionally active CAR disclosed in WO2014127261, herein incorporated by reference in its entirety. In some embodiments, the CCAR is a sortase synthesized CAR disclosed in WO2016014553, herein incorporated by reference in its entirety.

A “regulatory molecule,” as used herein, refers to a molecule that has a regulatory activity or a molecule that can be used to mediate a regulatory activity. In some embodiments, the regulatory molecule can be co-expressed with a CAR in a cell to regulate the expression and/or activity of the CAR, either directly (e.g., by directly affecting the expression level or functional activity of the CAR) or indirectly (e.g., by regulating the survival or activity of the cell expressing the CAR). In some embodiments, the regulatory molecule can be used to induce death, e.g., induce apoptosis, of a cell, e.g., a CAR-expressing cell. In some embodiments, the regulatory molecule can be used to activate a cell, e.g., a CAR-expressing cell. In some embodiments, the regulatory molecule is a marker, e.g., a cell surface marker, that labels a cell, e.g., a CAR-expressing cell, for depletion. In some embodiments, the regulatory molecule is a caspase, e.g., an inducible caspase 9, e.g., an inducible caspase 9 disclosed in WO2011146862, WO2014164348, or WO2016100236, herein incorporated by reference in their entireties. In some embodiments, the regulatory molecule is a truncated EGFR, e.g., a truncated EGFR disclosed in WO2011056894 or WO2013123061, incorporated herein by reference in their entireties.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

The compositions and methods of the present disclosure encompass polypeptides and nucleic acids having the sequences specified, or sequences substantially identical or similar thereto, for example, sequences at least 85%, 90%, or 95% identical or higher to the sequence specified. In the context of an amino acid sequence, the term “substantially identical” is used herein to refer to a first amino acid sequence that contains a sufficient or minimum number of amino acid residues that are i) identical to, or ii) conservative substitutions of aligned amino acid residues in a second amino acid sequence such that the first and second amino acid sequences can have a common structural domain and/or common functional activity, for example, amino acid sequences that contain a common structural domain having at least about 85%, 90%. 91%, 92%, 93%, 94%, 95%, 96%, 97%, 98% or 99% identity to a reference sequence, for example, a sequence provided herein.

In the context of a nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity, for example, nucleotide sequences having at least about 75%, 80%, 85%, 90%, 91%, 92%, 93%, 94%, 95%, 96%, 97%, 98% or 99% identity to a reference sequence, for example, a sequence provided herein.

The term “variant” refers to a polypeptide that has a substantially identical amino acid sequence to a reference amino acid sequence, or is encoded by a substantially identical nucleotide sequence. In some embodiments, the variant is a functional variant.

The term “functional variant” refers to a polypeptide that has a substantially identical amino acid sequence to a reference amino acid sequence, or is encoded by a substantially identical nucleotide sequence, and is capable of having one or more activities of the reference amino acid sequence.

The term cytokine (for example, IL-2, IL-7, IL-15, IL-21, or IL-6) includes full length, a fragment or a variant, for example, a functional variant, of a naturally-occurring cytokine (including fragments and functional variants thereof having at least 10%, 30%, 50%, or 80% of the activity, e.g., the immunomodulatory activity, of the naturally-occurring cytokine). In some embodiments, the cytokine has an amino acid sequence that is substantially identical (e.g., at least about 85%, 90%, 91%, 92%, 93%, 94%, 95%, 96%, 97%, 98% or 99% identity) to a naturally-occurring cytokine, or is encoded by a nucleotide sequence that is substantially identical (e.g., at least about 75%, 80%, 85%, 90%, 91%, 92%, 93%, 94%, 95%, 96%, 97%, 98% or 99% identity) to a naturally-occurring nucleotide sequence encoding a cytokine. In some embodiments, as understood in context, the cytokine further comprises a receptor domain, e.g., a cytokine receptor domain (e.g., an IL-15/IL-15R).

The term “Chimeric Antigen Receptor” or alternatively a “CAR” refers to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule as defined below. In some embodiments, the domains in the CAR polypeptide construct are in the same polypeptide chain, for example, comprise a chimeric fusion protein. In some embodiments, the domains in the CAR polypeptide construct are not contiguous with each other, for example, are in different polypeptide chains, for example, as provided in an RCAR as described herein. In some embodiments, the CAR is a CCAR, e.g., a CCAR disclosed herein.

In some embodiments, the cytoplasmic signaling domain comprises a primary signaling domain (for example, a primary signaling domain of CD3-zeta). In some embodiments, the cytoplasmic signaling domain further comprises one or more functional signaling domains derived from at least one costimulatory molecule as defined below. In some embodiments, the costimulatory molecule is chosen from 41BB (i.e., CD137), CD27, ICOS, and/or CD28. In some embodiments, the CAR comprises a chimeric fusion protein comprising an extracellular antigen recognition domain, a transmembrane domain and an intracellular signaling domain comprising a functional signaling domain derived from a stimulatory molecule. In some embodiments, the CAR comprises a chimeric fusion protein comprising an extracellular antigen recognition domain, a transmembrane domain and an intracellular signaling domain comprising a functional signaling domain derived from a costimulatory molecule and a functional signaling domain derived from a stimulatory molecule. In some embodiments, the CAR comprises a chimeric fusion protein comprising an extracellular antigen recognition domain, a transmembrane domain and an intracellular signaling domain comprising two functional signaling domains derived from one or more costimulatory molecule(s) and a functional signaling domain derived from a stimulatory molecule. In some embodiments, the CAR comprises a chimeric fusion protein comprising an extracellular antigen recognition domain, a transmembrane domain and an intracellular signaling domain comprising at least two functional signaling domains derived from one or more costimulatory molecule(s) and a functional signaling domain derived from a stimulatory molecule. In some embodiments the CAR comprises an optional leader sequence at the amino-terminus (N-terminus) of the CAR fusion protein. In some embodiments, the CAR further comprises a leader sequence at the N-terminus of the extracellular antigen recognition domain, wherein the leader sequence is optionally cleaved from the antigen recognition domain (for example, an scFv) during cellular processing and localization of the CAR to the cellular membrane.

A CAR that comprises an antigen binding domain (for example, an scFv, a single domain antibody, or TCR (for example, a TCR alpha binding domain or TCR beta binding domain)) that targets a specific tumor marker X, wherein X can be a tumor marker as described herein, is also referred to as XCAR. For example, a CAR that comprises an antigen binding domain that targets BCMA is referred to as BCMA CAR. The CAR can be expressed in any cell, for example, an immune effector cell as described herein (for example, a T cell or an NK cell).

The term “signaling domain” refers to the functional portion of a protein which acts by transmitting information within the cell to regulate cellular activity via defined signaling pathways by generating second messengers or functioning as effectors by responding to such messengers.

The term “antibody,” as used herein, refers to a protein, or polypeptide sequence derived from an immunoglobulin molecule, which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

The term “antibody fragment” refers to at least one portion of an intact antibody, or recombinant variants thereof, and refers to the antigen binding domain, for example, an antigenic determining variable region of an intact antibody, that is sufficient to confer recognition and specific binding of the antibody fragment to a target, such as an antigen. Examples of antibody fragments include, but are not limited to, Fab, Fab, F(ab)2, and Fv fragments, scFv antibody fragments, linear antibodies, single domain antibodies such as sdAb (either VL or VH), camelid VHH domains, and multi-specific molecules formed from antibody fragments such as a bivalent fragment comprising two or more, for example, two, Fab fragments linked by a disulfide bridge at the hinge region, or two or more, for example, two isolated CDR or other epitope binding fragments of an antibody linked. An antibody fragment can also be incorporated into single domain antibodies, maxibodies, minibodies, nanobodies, intrabodies, diabodies, triabodies, tetrabodies, v-NAR and bis-scFv (see, for example, Hollinger and Hudson, Nature Biotechnology 23:1126-1136, 2005). Antibody fragments can also be grafted into scaffolds based on polypeptides such as a fibronectin type III (Fn3) (see U.S. Pat. No. 6,703,199, which describes fibronectin polypeptide minibodies).

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, for example, with respect to the N-terminal and C-terminal ends of the polypeptide, the scFv may comprise VL-linker-VH or may comprise VH-linker-VL. In some embodiments, the scFv may comprise the structure of NH₂-V_{L}-linker-V_{H}-COOH or NH₂-V_{H}-linker-V_{L}-COOH.

The terms “complementarity determining region” or “CDR,” as used herein, refer to the sequences of amino acids within antibody variable regions which confer antigen specificity and binding affinity. For example, in general, there are three CDRs in each heavy chain variable region (for example, HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme), or a combination thereof. In a combined Kabat and Chothia numbering scheme, in some embodiments, the CDRs correspond to the amino acid residues that are part of a Kabat CDR, a Chothia CDR, or both.

The portion of the CAR composition of this disclosure comprising an antibody or antibody fragment thereof may exist in a variety of forms, for example, where the antigen binding domain is expressed as part of a polypeptide chain including, for example, a single domain antibody fragment (sdAb), a single chain antibody (scFv), or for example, a human or humanized antibody (Harlow et al., 20 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426). In some embodiments, the antigen binding domain of a CAR composition of this disclosure comprises an antibody fragment. In some embodiments, the CAR comprises an antibody fragment that comprises an scFv.

As used herein, the term “binding domain” or “antibody molecule” (also referred to herein as “anti-target binding domain”) refers to a protein, for example, an immunoglobulin chain or fragment thereof, comprising at least one immunoglobulin variable domain sequence. The term “binding domain” or “antibody molecule” encompasses antibodies and antibody fragments. In some embodiments, an antibody molecule is a multispecific antibody molecule, for example, it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In some embodiments, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope.

The terms “bispecific antibody” and “bispecific antibodies” refer to molecules that combine the antigen binding sites of two antibodies within a single molecule. Thus, a bispecific antibody is able to bind two different antigens simultaneously or sequentially. Methods for making bispecific antibodies are well known in the art. Various formats for combining two antibodies are also known in the art. Forms of bispecific antibodies of this disclosure include, but are not limited to, a diabody, a single-chain diabody, Fab dimerization (Fab-Fab), Fab-scFv, and a tandem antibody, as known to those of skill in the art.

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (κ) and lambda (λ) light chains refer to the two major antibody light chain isotypes.

The term “recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present disclosure includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

The terms “anti-tumor effect” and “anti-cancer effect” are used interchangeably and refer to a biological effect which can be manifested by various means, including but not limited to, for example, a decrease in tumor volume or cancer volume, a decrease in the number of tumor cells or cancer cells, a decrease in the number of metastases, an increase in life expectancy, a decrease in tumor cell proliferation or cancer cell proliferation, a decrease in tumor cell survival or cancer cell survival, or amelioration of various physiological symptoms associated with the cancerous condition. An “anti-tumor effect” or “anti-cancer effect” can also be manifested by the ability of the peptides, polynucleotides, cells and antibodies of this disclosure in prevention of the occurrence of tumor or cancer in the first place.

The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some embodiments, allogeneic material from individuals of the same species may be sufficiently unlike genetically to interact antigenically.

The term “xenogeneic” refers to a graft derived from an animal of a different species.

The term “apheresis” as used herein refers to the art-recognized extracorporeal process by which the blood of a donor or patient is removed from the donor or patient and passed through an apparatus that separates out selected particular constituent(s) and returns the remainder to the circulation of the donor or patient, for example, by retransfusion. Thus, in the context of “an apheresis sample” refers to a sample obtained using apheresis.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In some embodiments cancers treated by the methods described herein include multiple myeloma, Hodgkin's lymphoma or non-Hodgkin's lymphoma.

The terms “tumor” and “cancer” are used interchangeably herein, for example, both terms encompass solid and liquid, for example, diffuse or circulating, tumors. As used herein, the term “cancer” or “tumor” includes premalignant, as well as malignant cancers and tumors.

“Derived from” as that term is used herein, indicates a relationship between a first and a second molecule. It generally refers to structural similarity between the first molecule and a second molecule and does not connotate or include a process or source limitation on a first molecule that is derived from a second molecule. For example, in the case of an intracellular signaling domain that is derived from a CD3zeta molecule, the intracellular signaling domain retains sufficient CD3zeta structure such that is has the required function, namely, the ability to generate a signal under the appropriate conditions. It does not connotate or include a limitation to a particular process of producing the intracellular signaling domain, for example, it does not mean that, to provide the intracellular signaling domain, one must start with a CD3zeta sequence and delete unwanted sequence, or impose mutations, to arrive at the intracellular signaling domain.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of this disclosure by standard techniques known in the art, such as site-directed mutagenesis and PCR-mediated mutagenesis. Conservative substitutions are ones in which the amino acid residue is replaced with an amino acid residue having a similar side chain. Families of amino acid residues having similar side chains have been defined in the art. These families include amino acids with basic side chains (for example, lysine, arginine, histidine), acidic side chains (for example, aspartic acid, glutamic acid), uncharged polar side chains (for example, glycine, asparagine, glutamine, serine, threonine, tyrosine, cysteine, tryptophan), nonpolar side chains (for example, alanine, valine, leucine, isoleucine, proline, phenylalanine, methionine), beta-branched side chains (for example, threonine, valine, isoleucine) and aromatic side chains (for example, tyrosine, phenylalanine, tryptophan, histidine). Thus, one or more amino acid residues within a CAR of this disclosure can be replaced with other amino acid residues from the same side chain family and the altered CAR can be tested using the functional assays described herein.

The term “stimulation” in the context of stimulation by a stimulatory and/or costimulatory molecule refers to a response, for example, a primary or secondary response, induced by binding of a stimulatory molecule (for example, a TCR/CD3 complex) and/or a costimulatory molecule (for example, CD28 or 4-1BB) with its cognate ligand thereby mediating a signal transduction event, such as, but not limited to, signal transduction via the TCR/CD3 complex. Stimulation can mediate altered expression of certain molecules and/or reorganization of cytoskeletal structures, and the like.

The term “stimulatory molecule,” refers to a molecule expressed by a T cell that provides the primary cytoplasmic signaling sequence(s) that regulate primary activation of the TCR complex in a stimulatory way for at least some aspect of the T cell signaling pathway. In some embodiments, the ITAM-containing domain within the CAR recapitulates the signaling of the primary TCR independently of endogenous TCR complexes. In some embodiments, the primary signal is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM. Examples of an ITAM containing primary cytoplasmic signaling sequence that is of particular use in this disclosure includes, but is not limited to, those derived from TCR zeta, FcR gamma, FcR beta, CD3 gamma, CD3 delta, CD3 epsilon, CD5, CD22, CD79a, CD79b, CD278 (also known as “ICOS”), FcεRI and CD66d, DAP10 and DAP12. In a specific CAR of this disclosure, the intracellular signaling domain in any one or more CARS of this disclosure comprises an intracellular signaling sequence, for example, a primary signaling sequence of CD3-zeta. The term “antigen presenting cell” or “APC” refers to an immune system cell such as an accessory cell (for example, a B-cell, a dendritic cell, and the like) that displays a foreign antigen complexed with major histocompatibility complexes (MHCs) on its surface. T-cells may recognize these complexes using their T-cell receptors (TCRs). APCs process antigens and present them to T-cells.

An “intracellular signaling domain,” as the term is used herein, refers to an intracellular portion of a molecule. In embodiments, the intracellular signal domain transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

The intracellular signaling domain generates a signal that promotes an immune effector function of the CAR containing cell, for example, a CART cell. Examples of immune effector function, for example, in a CART cell, include cytolytic activity and helper activity, including the secretion of cytokines.

In some embodiments, the intracellular signaling domain can comprise a primary intracellular signaling domain. Exemplary primary intracellular signaling domains include those derived from the molecules responsible for primary stimulation, or antigen dependent simulation. In some embodiments, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, CD278 (also known as “ICOS”), FcεRI, CD66d, DAP10 and DAP12.

The term “zeta” or alternatively “zeta chain”, “CD3-zeta” or “TCR-zeta” refers to CD247. Swiss-Prot accession number P20963 provides exemplary human CD3 zeta amino acid sequences. A “zeta stimulatory domain” or alternatively a “CD3-zeta stimulatory domain” or a “TCR-zeta stimulatory domain” refers to a stimulatory domain of CD3-zeta or a variant thereof (for example, a molecule having mutations, for example, point mutations, fragments, insertions, or deletions). In some embodiments, the cytoplasmic domain of zeta comprises residues 52 through 164 of GenBank Acc. No. BAG36664.1 or a variant thereof (for example, a molecule having mutations, for example, point mutations, fragments, insertions, or deletions). In some embodiments, the “zeta stimulatory domain” or a “CD3-zeta stimulatory domain” is the sequence provided as SEQ ID NO: 9 or 10, or a variant thereof (for example, a molecule having mutations, for example, point mutations, fragments, insertions, or deletions).

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to an MHC class I molecule, TNF receptor proteins, Immunoglobulin-like proteins, cytokine receptors, integrins, signaling lymphocytic activation molecules (SLAM proteins), activating NK cell receptors, BTLA, Toll ligand receptor, OX40, CD2, CD7, CD27, CD28, CD30, CD40, CD5, ICAM-1, LFA-1 (CD11a/CD18), 4-1BB (CD137), B7-H3, CD5, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD28-OX40, CD28-4-1BB, and a ligand that specifically binds with CD83.

A costimulatory intracellular signaling domain refers to the intracellular portion of a costimulatory molecule.

The intracellular signaling domain can comprise the entire intracellular portion, or the entire native intracellular signaling domain, of the molecule from which it is derived, or a functional fragment thereof.

The term “4-1BB” refers to CD137 or Tumor necrosis factor receptor superfamily member 9. Swiss-Prot accession number P20963 provides exemplary human 4-1BB amino acid sequences. A “4-1BB costimulatory domain” refers to a costimulatory domain of 4-1BB, or a variant thereof (for example, a molecule having mutations, for example, point mutations, fragments, insertions, or deletions). In some embodiments, the “4-1BB costimulatory domain” is the sequence provided as SEQ ID NO: 7 or a variant thereof (for example, a molecule having mutations, for example, point mutations, fragments, insertions, or deletions).

“Immune effector cell,” as that term is used herein, refers to a cell that is involved in an immune response, for example, in the promotion of an immune effector response. Examples of immune effector cells include T cells, for example, alpha/beta T cells and gamma/delta T cells, B cells, natural killer (NK) cells, natural killer T (NKT) cells, mast cells, and myeloic-derived phagocytes.

“Immune effector function or immune effector response,” as that term is used herein, refers to function or response, for example, of an immune effector cell, that enhances or promotes an immune attack of a target cell. For example, an immune effector function or response refers a property of a T or NK cell that promotes killing or the inhibition of growth or proliferation, of a target cell. In the case of a T cell, primary stimulation and costimulation are examples of immune effector function or response.

The term “effector function” refers to a specialized function of a cell. Effector function of a T cell, for example, may be cytolytic activity or helper activity including the secretion of cytokines.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (for example, rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “effective amount” or “therapeutically effective amount” are used interchangeably herein, and refer to an amount of a compound, formulation, material, or composition, as described herein effective to achieve a particular biological result.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence. In some embodiments, expression comprises translation of an mRNA introduced into a cell.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (for example, naked or contained in liposomes) and viruses (for example, lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, for example, the LENTIVECTOR® gene delivery technology from Oxford BioMedica, the LENTIMAX™ vector system from Lentigen and the like. Nonclinical types of lentiviral vectors are also available and would be known to one skilled in the art.

The term “homologous” or “identity” refers to the subunit sequence identity between two polymeric molecules, for example, between two nucleic acid molecules, such as, two DNA molecules or two RNA molecules, or between two polypeptide molecules. When a subunit position in both of the two molecules is occupied by the same monomeric subunit; for example, if a position in each of two DNA molecules is occupied by adenine, then they are homologous or identical at that position. The homology between two sequences is a direct function of the number of matching or homologous positions; for example, if half (for example, five positions in a polymer ten subunits in length) of the positions in two sequences are homologous, the two sequences are 50% homologous; if 90% of the positions (for example, 9 of 10), are matched or homologous, the two sequences are 90% homologous.

“Humanized” forms of non-human (for example, murine) antibodies are chimeric immunoglobulins, immunoglobulin chains or fragments thereof (such as Fv, Fab, Fab, F(ab)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, for example, where necessary to join two protein coding regions, are in the same reading frame.

The term “parenteral” administration of an immunogenic composition includes, for example, subcutaneous (s.c.), intravenous (i.v.), intramuscular (i.m.), or intrasternal injection, intratumoral, or infusion techniques.

The term “nucleic acid,” “nucleic acid molecule,” “polynucleotide,” or “polynucleotide molecule” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. In some embodiments, a “nucleic acid,” “nucleic acid molecule,” “polynucleotide,” or “polynucleotide molecule” comprise a nucleotide/nucleoside derivative or analog. Unless otherwise indicated, a particular nucleic acid sequence also implicitly encompasses conservatively modified variants thereof (for example, degenerate codon substitutions, for example, conservative substitutions), alleles, orthologs, SNPs, and complementary sequences as well as the sequence explicitly indicated. Specifically, degenerate codon substitutions, for example, conservative substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The terms “cancer associated antigen,” “tumor antigen,” “hyperproliferative disorder antigen,” and “antigen associated with a hyperproliferative disorder” interchangeably refer to antigens that are common to specific hyperproliferative disorders. In some embodiments, these terms refer to a molecule (typically a protein, carbohydrate or lipid) that is expressed on the surface of a cancer cell, either entirely or as a fragment (for example, MHC/peptide), and which is useful for the preferential targeting of a pharmacological agent to the cancer cell. In some embodiments, a tumor antigen is a marker expressed by both normal cells and cancer cells, for example, a lineage marker, for example, CD19 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (for example, MHC/peptide), and not synthesized or expressed on the surface of a normal cell. In some embodiments, the hyperproliferative disorder antigens of the present disclosure are derived from, cancers including but not limited to primary or metastatic melanoma, thymoma, lymphoma, sarcoma, lung cancer, liver cancer, non-Hodgkin lymphoma, Hodgkin lymphoma, leukemias, uterine cancer, cervical cancer, bladder cancer, kidney cancer and adenocarcinomas such as breast cancer, prostate cancer (for example, castrate-resistant or therapy-resistant prostate cancer, or metastatic prostate cancer), ovarian cancer, pancreatic cancer, and the like, or a plasma cell proliferative disorder, for example, asymptomatic myeloma (smoldering multiple myeloma or indolent myeloma), monoclonal gammopathy of undetermined significance (MGUS), Waldenstrom's macroglobulinemia, plasmacytomas (for example, plasma cell dyscrasia, solitary myeloma, solitary plasmacytoma, extramedullary plasmacytoma, and multiple plasmacytoma), systemic amyloid light chain amyloidosis, and POEMS syndrome (also known as Crow-Fukase syndrome, Takatsuki disease, and PEP syndrome). In some embodiments, the CARs of the present disclosure include CARs comprising an antigen binding domain (for example, antibody or antibody fragment) that binds to a MHC presented peptide. Normally, peptides derived from endogenous proteins fill the pockets of Major histocompatibility complex (MHC) class I molecules and are recognized by T cell receptors (TCRs) on CD8+T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, for example, Sastry et al., J Virol. 2011 85(5):1935-1942; Sergeeva et al., Blood, 2011 117(16):4262-4272; Verma et al., J Immunol 2010 184(4):2156-2165; Willemsen et al., Gene Ther 2001 8(21) :1601-1608; Dao et al., Sci Transl Med 2013 5(176) :176ra33 ; Tassev et al., Cancer Gene Ther 2012 19(2):84-100). For example, TCR-like antibody can be identified from screening a library, such as a human scFv phage displayed library.

The term “tumor-supporting antigen” or “cancer-supporting antigen” interchangeably refer to a molecule (typically a protein, carbohydrate or lipid) that is expressed on the surface of a cell that is, itself, not cancerous, but supports the cancer cells, for example, by promoting their growth or survival for example, resistance to immune cells. Exemplary cells of this type include stromal cells and myeloid-derived suppressor cells (MDSCs). The tumor-supporting antigen itself need not play a role in supporting the tumor cells so long as the antigen is present on a cell that supports cancer cells.

The term “flexible polypeptide linker” or “linker” as used in the context of an scFv refers to a peptide linker that consists of amino acids such as glycine and/or serine residues used alone or in combination, to link variable heavy and variable light chain regions together. In some embodiments, the flexible polypeptide linker is a Gly/Ser linker and comprises the amino acid sequence (Gly-Gly-Gly-Ser)n, where n is a positive integer equal to or greater than 1 (SEQ ID NO: 41). For example, n=1, n=2, n=3. n=4, n=5 and n=6, n=7, n=8, n=9 and n=10 In some embodiments, the flexible polypeptide linkers include, but are not limited to, (Gly4 Ser)4 (SEQ ID NO: 27) or (Gly4 Ser)3 (SEQ ID NO: 28). In some embodiments, the linkers include multiple repeats of (Gly2Ser), (GlySer) or (Gly3Ser) (SEQ ID NO: 29). Also included within the scope of the present disclosure are linkers described in WO2012/138475, incorporated herein by reference.

As used herein, a 5 CIap (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5 Rap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5 Rnd of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA that has been synthesized in vitro. In some embodiments the RNA is mRNA. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In some embodiments of a construct for transient expression, the poly(A) is between 50 and 5000 (SEQ ID NO: 30). In some embodiments the poly(A) is greater than 64. In some embodiments the poly(A)is greater than 100. In some embodiments the poly(A) is greater than 300. In some embodiments the poly(A) is greater than 400. poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3 end. The 3 poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3 end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more therapies (for example, one or more therapeutic agents such as a CAR of the present disclosure). In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating” -refer to the inhibition of the progression of a proliferative disorder, either physically by, for example, stabilization of a discernible symptom, physiologically by, for example, stabilization of a physical parameter, or both. In other embodiments the terms “treat”, “treatment” and “treating” refer to the reduction or stabilization of tumor size or cancerous cell count.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (for example, mammals, for example, human).

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In some embodiments, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, or a ligand, which recognizes and binds with a cognate binding partner (for example, a stimulatory and/or costimulatory molecule present on a T cell) protein present in a sample, but which antibody or ligand does not substantially recognize or bind other molecules in the sample.

“Regulatable chimeric antigen receptor (RCAR),” as used herein, refers to a set of polypeptides, typically two in the simplest embodiments, which when in an immune effector cell, provides the cell with specificity for a target cell, typically a cancer cell, and with intracellular signal generation. In some embodiments, an RCAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined herein in the context of a CAR molecule. In some embodiments, the set of polypeptides in the RCAR are not contiguous with each other, for example, are in different polypeptide chains. In some embodiments, the RCAR includes a dimerization switch that, upon the presence of a dimerization molecule, can couple the polypeptides to one another, for example, can couple an antigen binding domain to an intracellular signaling domain. In some embodiments, the RCAR is expressed in a cell (for example, an immune effector cell) as described herein, for example, an RCAR-expressing cell (also referred to herein as “RCARX cell”). In some embodiments the RCARX cell is a T cell and is referred to as an RCART cell. In some embodiments the RCARX cell is an NK cell, and is referred to as an RCARN cell. The RCAR can provide the RCAR-expressing cell with specificity for a target cell, typically a cancer cell, and with regulatable intracellular signal generation or proliferation, which can optimize an immune effector property of the RCAR-expressing cell. In embodiments, an RCAR cell relies at least in part, on an antigen binding domain to provide specificity to a target cell that comprises the antigen bound by the antigen binding domain.

“Membrane anchor” or “membrane tethering domain”, as that term is used herein, refers to a polypeptide or moiety, for example, a myristoyl group, sufficient to anchor an extracellular or intracellular domain to the plasma membrane.

“Switch domain,” as that term is used herein, for example, when referring to an RCAR, refers to an entity, typically a polypeptide-based entity, that, in the presence of a dimerization molecule, associates with another switch domain. The association results in a functional coupling of a first entity linked to, for example, fused to, a first switch domain, and a second entity linked to, for example, fused to, a second switch domain. A first and second switch domain are collectively referred to as a dimerization switch. In embodiments, the first and second switch domains are the same as one another, for example, they are polypeptides having the same primary amino acid sequence and are referred to collectively as a homodimerization switch. In embodiments, the first and second switch domains are different from one another, for example, they are polypeptides having different primary amino acid sequences, and are referred to collectively as a heterodimerization switch. In embodiments, the switch is intracellular. In embodiments, the switch is extracellular. In embodiments, the switch domain is a polypeptide-based entity, for example, FKBP or FRB-based, and the dimerization molecule is small molecule, for example, a rapalogue. In embodiments, the switch domain is a polypeptide-based entity, for example, an scFv that binds a myc peptide, and the dimerization molecule is a polypeptide, a fragment thereof, or a multimer of a polypeptide, for example, a myc ligand or multimers of a myc ligand that bind to one or more myc scFvs. In embodiments, the switch domain is a polypeptide-based entity, for example, myc receptor, and the dimerization molecule is an antibody or fragments thereof, for example, myc antibody.

“Dimerization molecule,” as that term is used herein, for example, when referring to an RCAR, refers to a molecule that promotes the association of a first switch domain with a second switch domain. In embodiments, the dimerization molecule does not naturally occur in the subject or does not occur in concentrations that would result in significant dimerization. In embodiments, the dimerization molecule is a small molecule, for example, rapamycin or a rapalogue, for example, RAD001.

The term “low, immune enhancing, dose” when used in conjunction with an mTOR inhibitor, for example, an allosteric mTOR inhibitor, for example, RAD001 or rapamycin, or a catalytic mTOR inhibitor, refers to a dose of mTOR inhibitor that partially, but not fully, inhibits mTOR activity, for example, as measured by the inhibition of P70 S6 kinase activity. Methods for evaluating mTOR activity, for example, by inhibition of P70 S6 kinase, are discussed herein. The dose is insufficient to result in complete immune suppression but is sufficient to enhance the immune response. In some embodiments, the low, immune enhancing, dose of mTOR inhibitor results in a decrease in the number of PD-1 positive T cells and/or an increase in the number of PD-1 negative T cells, or an increase in the ratio of PD-1 negative T cells/PD-1 positive T cells. In some embodiments, the low, immune enhancing, dose of mTOR inhibitor results in an increase in the number of naïve T cells. In some embodiments, the low, immune enhancing, dose of mTOR inhibitor results in one or more of the following:

an increase in the expression of one or more of the following markers: CD62L^{high}, CD127^{high}, CD27⁺, and BCL2, for example, on memory T cells, for example, memory T cell precursors;

a decrease in the expression of KLRG1, for example, on memory T cells, for example, memory T cell precursors; and

an increase in the number of memory T cell precursors, for example, cells with any one or combination of the following characteristics: increased CD62L^{high} increased CD127^{high} increased CD27⁺, decreased KLRG1, and increased BCL2;

wherein any of the changes described above occurs, for example, at least transiently, for example, as compared to a non-treated subject.

“Refractory” as used herein refers to a disease, for example, cancer, that does not respond to a treatment. In embodiments, a refractory cancer can be resistant to a treatment before or at the beginning of the treatment. In other embodiments, the refractory cancer can become resistant during a treatment. A refractory cancer is also called a resistant cancer.

“Relapsed” or “relapse” as used herein refers to the return or reappearance of a disease (for example, cancer) or the signs and symptoms of a disease such as cancer after a period of improvement or responsiveness, for example, after prior treatment of a therapy, for example, cancer therapy. The initial period of responsiveness may involve the level of cancer cells falling below a certain threshold, for example, below 20%, 1%, 10%, 5%, 4%, 3%, 2%, or 1%. The reappearance may involve the level of cancer cells rising above a certain threshold, for example, above 20%, 1%, 10%, 5%, 4%, 3%, 2%, or 1%. For example, for example, in the context of B-ALL, the reappearance may involve, for example, a reappearance of blasts in the blood, bone marrow (>5%), or any extramedullary site, after a complete response. A complete response, in this context, may involve <5% BM blast. More generally, in some embodiments, a response (for example, complete response or partial response) can involve the absence of detectable MRD (minimal residual disease). In some embodiments, the initial period of responsiveness lasts at least 1, 2, 3, 4, 5, or 6 days; at least 1, 2, 3, or 4 weeks; at least 1, 2, 3, 4, 6, 8, 10, or 12 months; or at least 1, 2, 3, 4, or 5 years.

Ranges: throughout this disclosure, various embodiments of this disclosure can be presented in a range format. It should be understood that the description in range format is merely for convenience and brevity and should not be construed as an inflexible limitation on the scope of this disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98%, or 99% identity, and includes subranges such as 96-99%, 96-98%, 96-97%, 97-99%, 97-98%, and 98-99% identity. This applies regardless of the breadth of the range.

A “gene editing system” as the term is used herein, refers to a system, for example, one or more molecules, that direct and effect an alteration, for example, a deletion, of one or more nucleic acids at or near a site of genomic DNA targeted by said system. Gene editing systems are known in the art and are described more fully below.

Administered “in combination”, as used herein, means that two (or more) different treatments are delivered to the subject during the course of the subject affliction with the disorder, for example, the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, for example, an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The term “depletion” or “depleting”, as used interchangeably herein, refers to the decrease or reduction of the level or amount of a cell, a protein, or macromolecule in a sample after a process, for example, a selection step, for example, a negative selection, is performed. The depletion can be a complete or partial depletion of the cell, protein, or macromolecule. In some embodiments, the depletion is at least a 1%, 2%, 5%, 10%, 15%, 20%, 25%, 30%, 35%, 40%, 45%, 50%, 55%, 60%, 65%, 70%, 75%, 80%, 85%, 90%, 95%, or 99% decrease or reduction of the level or amount of a cell, a protein, or macromolecule, as compared to the level or amount of the cell, protein or macromolecule in the sample before the process was performed.

As used herein, a “naïve T cell” refers to a T cell that is antigen-inexperienced. In some embodiments, an antigen-inexperienced T cell has encountered its cognate antigen in the thymus but not in the periphery. In some embodiments, naïve T cells are precursors of memory cells. In some embodiments, naïve T cells express both CD45RA and CCR7, but do not express CD45RO. In some embodiments, naïve T cells may be characterized by expression of CD62L, CD27, CCR7, CD45RA, CD28, and CD127, and the absence of CD95 or CD45RO isoform. In some embodiments, naïve T cells express CD62L, IL-7 receptor-α, IL-6 receptor, and CD132, but do not express CD25, CD44, CD69, or CD45RO. In some embodiments, naïve T cells express CD45RA, CCR7, and CD62L and do not express CD95 or IL-2 receptor β. In some embodiments, surface expression levels of markers are assessed using flow cytometry.

The term “central memory T cells” refers to a subset of T cells that in humans are CD45RO positive and express CCR7. In some embodiments, central memory T cells express CD95. In some embodiments, central memory T cells express IL-2R, IL-7R and/or IL-15R. In some embodiments, central memory T cells express CD45RO, CD95, IL-2 receptor β, CCR7, and CD62L. In some embodiments, surface expression levels of markers are assessed using flow cytometry.

The term “stem memory T cells,” “stem cell memory T cells,” “stem cell-like memory T cells,” “memory stem T cells,” “T memory stem cells,” “T stem cell memory cells” or “TSCM cells” refers to a subset of memory T cells with stem cell-like ability, for example, the ability to self-renew and/or the multipotent capacity to reconstitute memory and/or effector T cell subsets. In some embodiments, stem memory T cells express CD45RA, CD95, IL-2 receptor β, CCR7, and CD62L. In some embodiments, surface expression levels of markers are assessed using flow cytometry. In some embodiments, exemplary stem memory T cells are disclosed in Gattinoni et al., Nat Med. 2017 Jan. 6; 23(1): 18-27, herein incorporated by reference in its entirety.

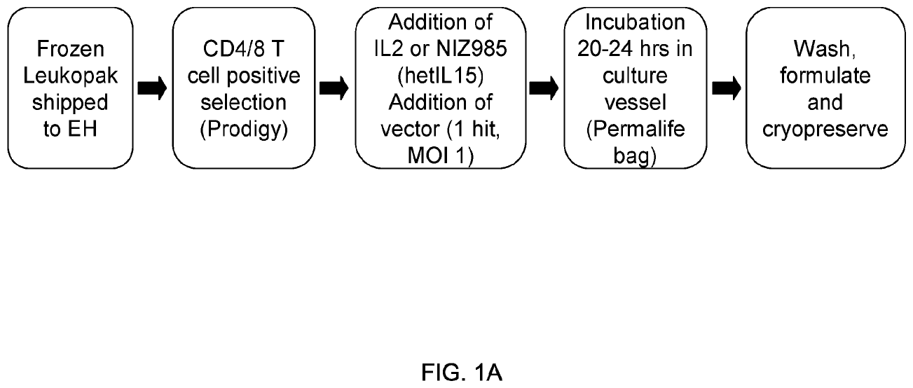

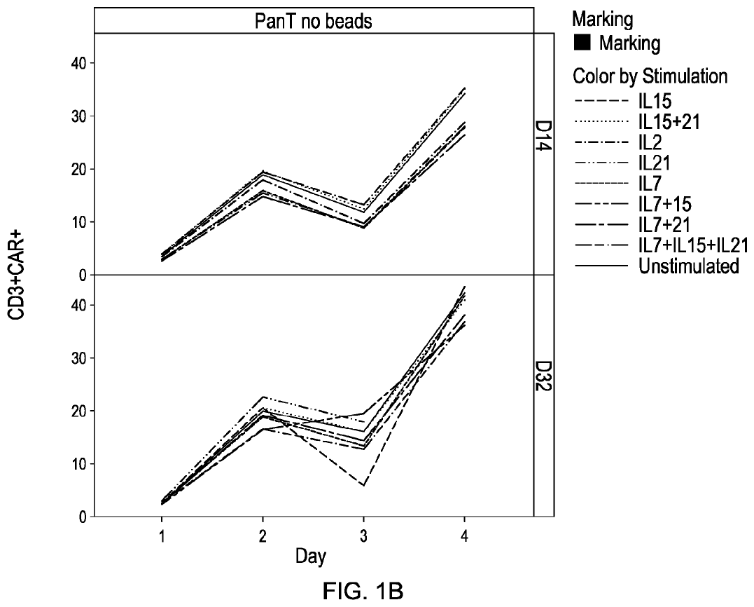

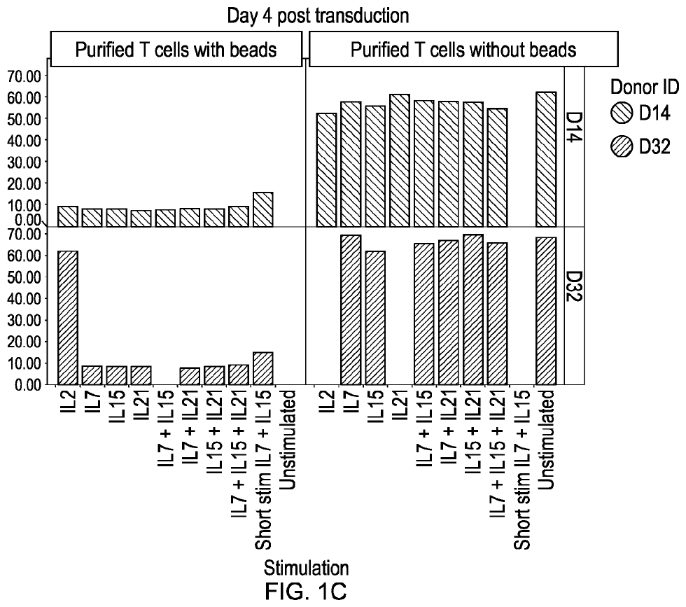

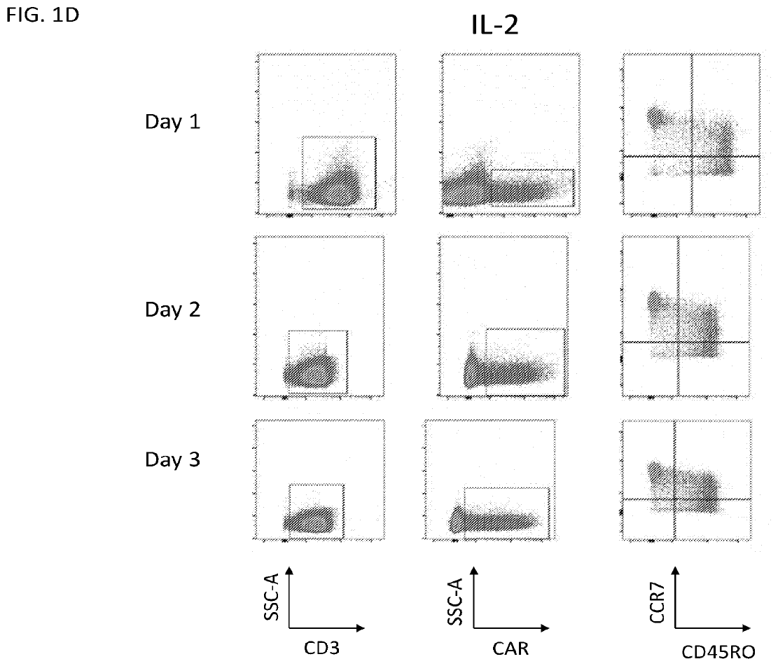

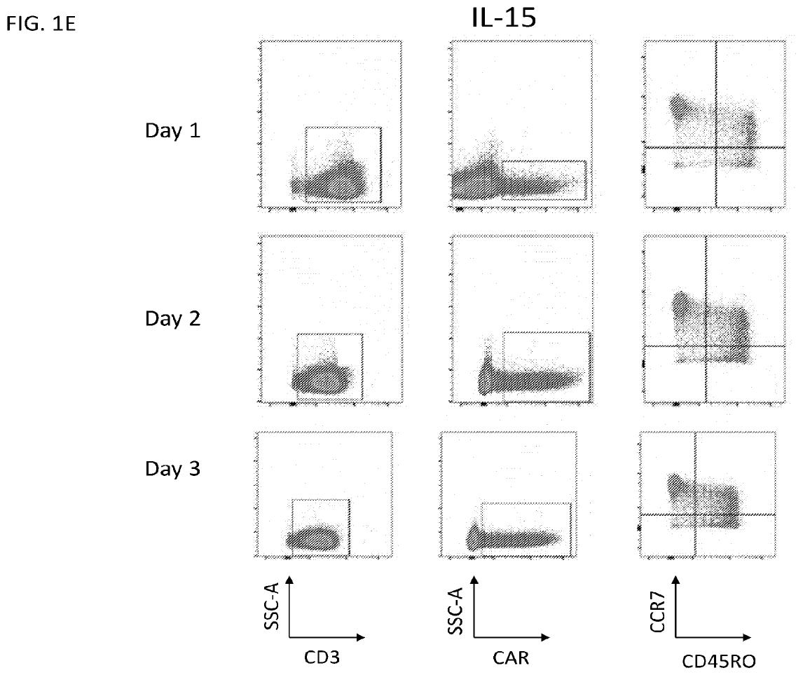

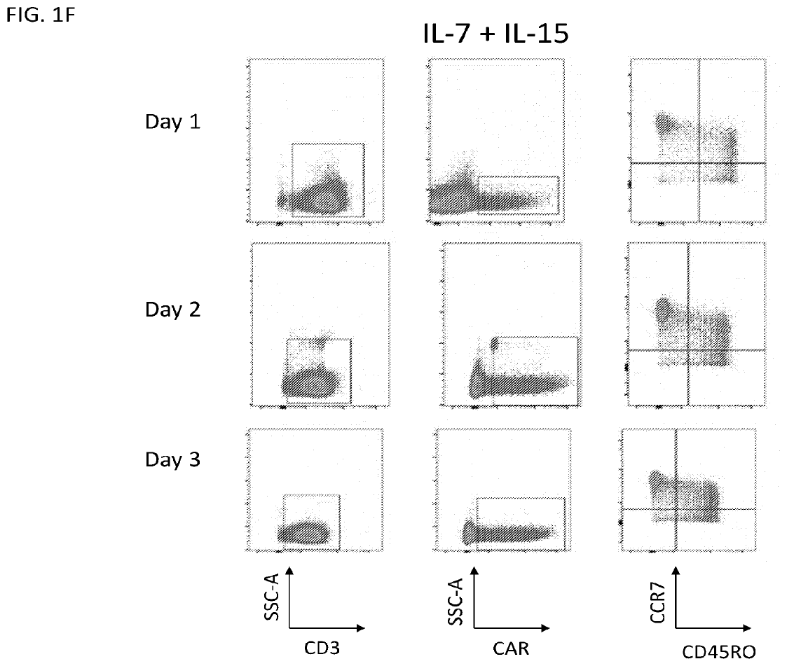

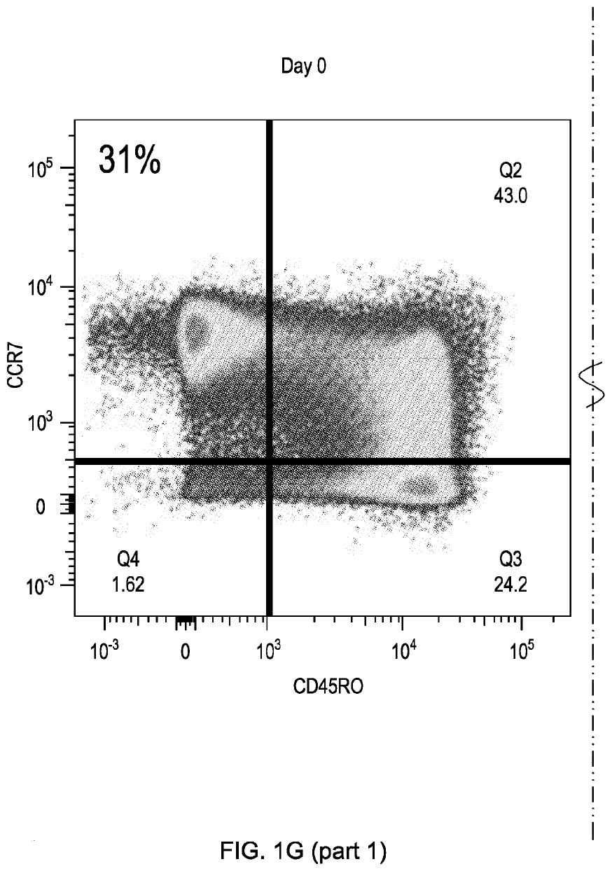

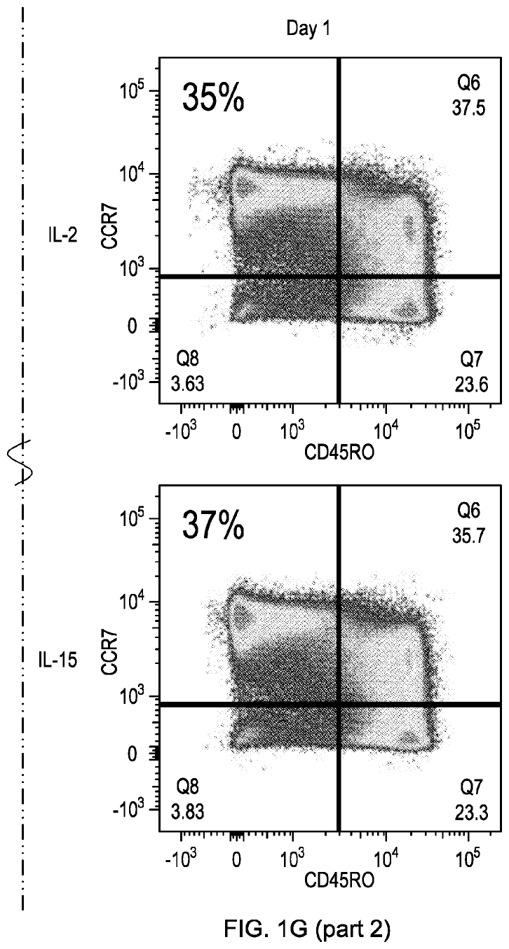

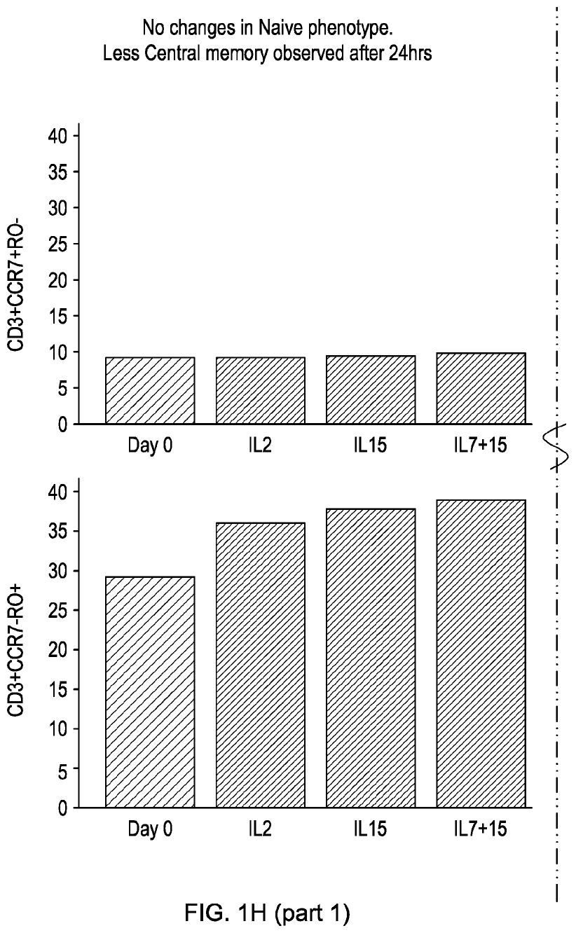

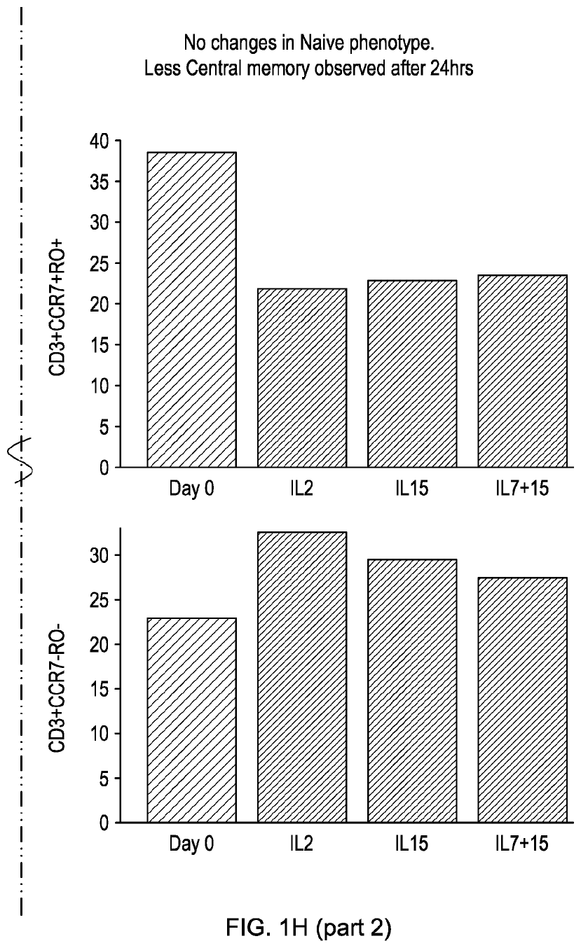

For clarity purposes, unless otherwise noted, classifying a cell or a population of cells as “not expressing,” or having an “absence of” or being “negative for” a particular marker may not necessarily mean an absolute absence of the marker. The skilled artisan can readily compare the cell against a positive and/or a negative control, and/or set a predetermined threshold, and classify the cell or population of cells as not expressing or being negative for the marker when the cell has an expression level below the predetermined threshold or a population of cells has an overall expression level below the predetermined threshold using conventional detection methods, e.g., using flow cytometry, for example, as described in the Examples herein. For example, representative gating strategies are shown in FIG. 1G. For example, CCR7 positive, CD45RO negative cells are shown in the top left quadrant in FIG. 1G.

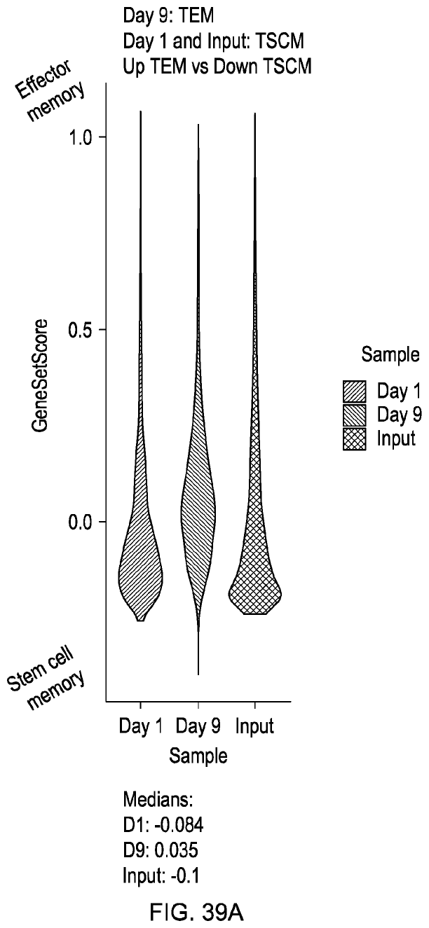

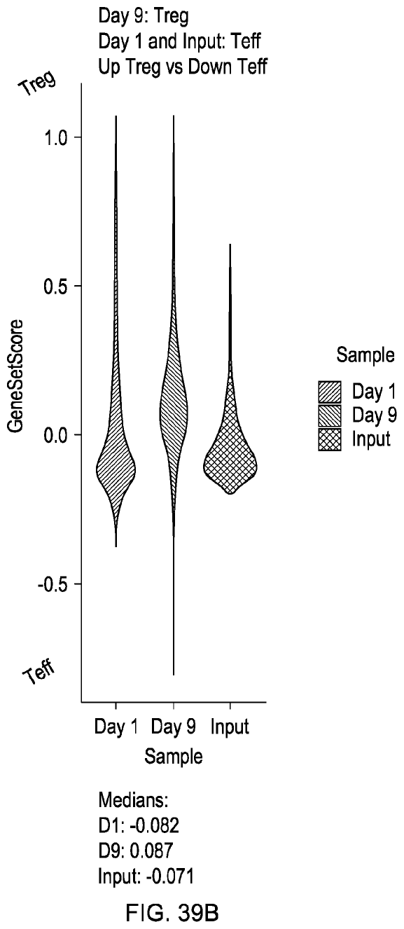

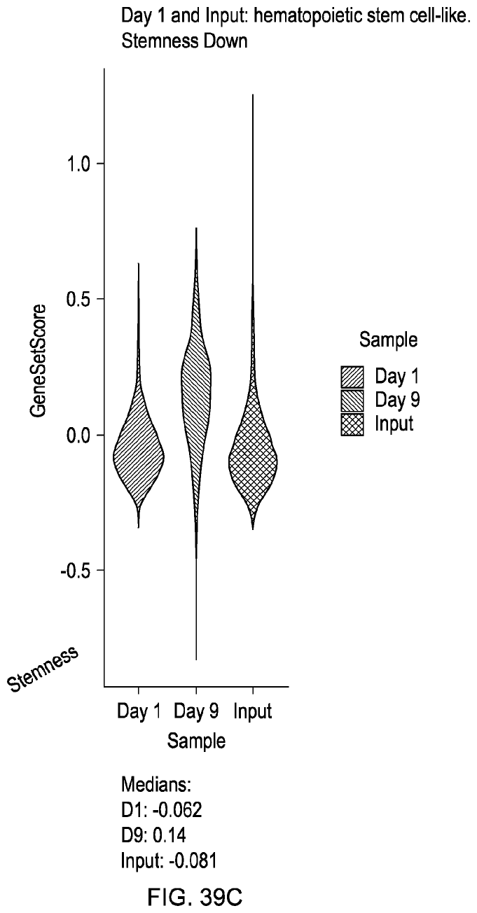

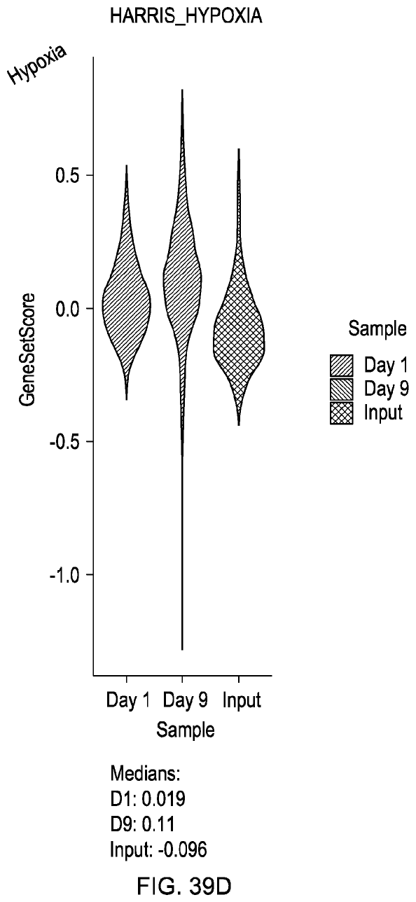

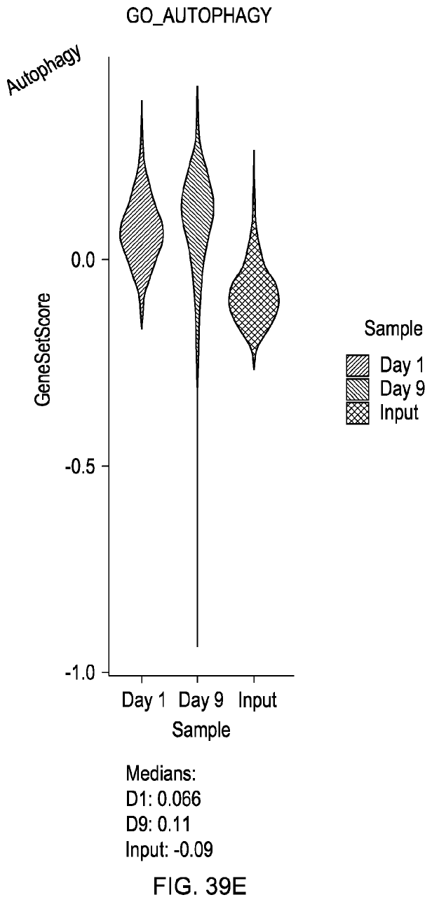

As used herein, the term “GeneSetScore (Up TEM vs. Down TSCM)” of a cell refers to a score that reflects the degree at which the cell shows an effector memory T cell (TEM) phenotype vs. a stem cell memory T cell (TSCM) phenotype. A higher GeneSetScore (Up TEM vs. Down TSCM) indicates an increasing TEM phenotype, whereas a lower GeneSetScore (Up TEM vs. Down TSCM) indicates an increasing TSCM phenotype. In some embodiments, the GeneSetScore (Up TEM vs. Down TSCM) is determined by measuring the expression of one or more genes that are up-regulated in TEM cells and/or down-regulated in TSCM cells, for example, one or more genes selected from the group consisting of MXRA7, CLIC1, NAT13, TBC1D2B, GLCCI1, DUSP10, APOBEC3D, CACNB3, ANXA2P2, TPRG1, EOMES, MATK, ARHGAP10, ADAMS, MAN1A1, SLFN12L, SH2D2A, EIF2C4, CD58, MYO1F, RAB27B, ERN1, NPC1, NBEAL2, APOBEC3G, SYTL2, SLC4A4, PIK3AP1, PTGDR, MAF, PLEKHAS, ADRB2, PLXND1, GNAO1, THBS1, PPP2R2B, CYTH3, KLRF1, FLJ16686, AUTS2, PTPRM, GNLY, and GFPT2. In some embodiments, the GeneSetScore (Up TEM vs. Down TSCM) is determined for each cell using RNA-seq, for example, single-cell RNA-seq (scRNA-seq), for example, as exemplified in Example 10 with respect to FIG. 39A. In some embodiments, the GeneSetScore (Up TEM vs. Down TSCM) is calculated by taking the mean log normalized gene expression value of all of the genes in the gene set.

As used herein, the term “GeneSetScore (Up Treg vs. Down Teff)” of a cell refers to a score that reflects the degree at which the cell shows a regulatory T cell (Treg) phenotype vs. an effector T cell (Teff) phenotype. A higher GeneSetScore (Up Treg vs. Down Teff) indicates an increasing Treg phenotype, whereas a lower GeneSetScore (Up Treg vs. Down Teff) indicates an increasing Teff phenotype. In some embodiments, the GeneSetScore (Up Treg vs. Down Teff) is determined by measuring the expression of one or more genes that are up-regulated in Treg cells and/or down-regulated in Teff cells, for example, one or more genes selected from the group consisting of C12orf75, SELPLG, SWAP70, RGS1, PRR11, SPATS2L, SPATS2L, TSHR, C14orf145, CASP8, SYT11, ACTN4, ANXAS, GLRX, HLA-DMB, PMCH, RAB11FIP1, IL32, FAM160B1, SHMT2, FRMD4B, CCR3, TNFRSF13B, NTNG2, CLDND1, BARD1, FCER1G, TYMS, ATP1B1, GJB6, FGL2, TK1, SLC2A8, CDKN2A, SKAP2, GPR55, CDCA7, S100A4, GDPDS, PMAIP1, ACOT9, CEP55, SGMS1, ADPRH, AKAP2, HDAC9, IKZF4, CARD17, VAV3, OBFC2A, ITGB1, CIITA, SETD7, HLA-DMA, CCR10, KIAA0101, SLC14A1, PTTG3P, DUSP10, FAM164A, PYHINL MYO1F, SLC1A4, MYBL2, PTTG1, RRM2, TP53INP1, CCRS, ST8SIA6, TOX, BFSP2, ITPRIPLL NCAPH, HLA-DPB2, SYT4, NINJ2, FAM46C, CCR4, GBPS, C15orf53, LMCD1, MKI67, NUSAP1, PDE4A, E2F2, CD58, ARHGEF12, LOC100188949, FAS, HLA-DPB1, SELP, WEE1, HLA-DPA1, FCRL1, ICA1, CNTNAP1, OAS1, METTL7A, CCR6, HLA-DRB4, ANXA2P3, STAM, HLA-DQB2, LGALS1, ANXA2, PI16, DUSP4, LAYN, ANXA2P2, PTPLA, ANXA2P1, ZNF365, LAIR2, L00541471, RASGRP4, BCAS1, UTS2, MIAT, PRDM1, SEMA3G, FAM129A, HPGD, NCF4, LGALS3, CEACAM4, JAKMIPL TIGIT, HLA-DRA, IKZF2, HLA-DRB1, FANK1, RTKN2, TRIB1, FCRL3, and FOXP3. In some embodiments, the GeneSetScore (Up Treg vs. Down Teff) is determined using RNA-seq, for example, single-cell RNA-seq (scRNA-seq), for example, as exemplified in Example 10 with respect to FIG. 39B. In some embodiments, the GeneSetScore (Up Treg vs. Down Teff) is calculated by taking the mean log normalized gene expression value of all of the genes in the gene set.

As used herein, the term “GeneSetScore (Down stemness)” of a cell refers to a score that reflects the degree at which the cell shows a stemness phenotype. A lower GeneSetScore (Down stemness) indicates an increasing stemness phenotype. In some embodiments, the GeneSetScore (Down stemness) is determined by measuring the expression of one or more genes that are upregulated in a differentiating stem cell vs downregulated in a hematopoietic stem cell, for example, one or more genes selected from the group consisting of ACE, BATF, CDK6, CHD2, ERCC2, HOXB4, MEOX1, SFRP1, SP7, SRF, TAL1, and XRCC5. In some embodiments, the GeneSetScore (Down stemness) is determined using RNA-seq, for example, single-cell RNA-seq (scRNA-seq), for example, as exemplified in Example 10 with respect to FIG. 39C. In some embodiments, the GeneSetScore (Down stemness) is calculated by taking the mean log normalized gene expression value of all of the genes in the gene set.

As used herein, the term “GeneSetScore (Up hypoxia)” of a cell refers to a score that reflects the degree at which the cell shows a hypoxia phenotype. A higher GeneSetScore (Up hypoxia) indicates an increasing hypoxia phenotype. In some embodiments, the GeneSetScore (Up hypoxia) is determined by measuring the expression of one or more genes that are up-regulated in cells undergoing hypoxia, for example, one or more genes selected from the group consisting of ABCB1, ACAT1, ADM, ADORA2B, 25 AK2, AK3, ALDH1A1, ALDH1A3, ALDOA, ALDOC, ANGPT2, ANGPTL4, ANXA1, ANXA2, ANXA5, ARHGAP5, ARSE, ART1, BACE2, BATF3, BCL2L1, BCL2L2, BHLHE40, BHLHE41, BIK, BIRC2, BNIP3, BNIP3L, BPI, BTG1, C11orf2, C7orf68, CA12, CA9, CALD1, CCNG2, CCT6A, CD99, CDK1, CDKN1A, CDKN1B, CITED2, CLK1, CNOT7, COL4A5, COL5A1, COL5A2, COL5A3, CP, CTSD, CXCR4, D4S234E, DDIT3, DDIT4, 1-Dec, DKC1, DR1, EDN1, EDN2, EFNA1, EGF, EGR1, EIF4A3, ELF3, ELL2, ENG, ENO1, ENO3, ENPEP, EPO, ERRFI1, ETS1, F3, FABPS, FGF3, FKBP4, FLT1, FN1, FOS, FTL, GAPDH, GBE1, GLRX, GPI, GPRCSA, HAP1, HBP1, HDAC1, HDAC9, HERC3, HERPUD1, HGF, HIF1A, HK1, HK2, HLA-DQB1, HMOX1, HMOX2, HSPAS, HSPD1, HSPH1, HYOU1, ICAM1, ID2, IFI27, IGF2, IGFBP1, IGFBP2, IGFBP3, IGFBP5, IL6, IL8, INSIG1, IRF6, ITGA5, JUN, KDR, KRT14, KRT18, KRT19, LDHA, LDHB, LEP, LGALS1, LONP1, LOX, LRP1, MAP4, MET, MIF, MMP13, MMP2, MMPI, MPI, MT1L, MTL3P, MUC1, MXI1, NDRG1, NFIL3, NFKB1, NFKB2, NOS1, NOS2, NOS2P1, NOS2P2, NOS3, NR3C1, NR4A1, NT5E, ODC1, P4HA1, P4HA2, PAICS, PDGFB, PDK3, PFKFB1, PFKFB3, PFKFB4, PFKL, PGAM1, PGF, PGK1, PGK2, PGM1, PIM1, PIM2, PKM2, PLAU, PLAUR, PLIN2, PLOD2, PNN, PNP, POLM, PPARA, PPAT, PROK1, PSMA3, PSMD9, PTGS1, PTGS2, QSOX1, RBPJ, RELA, RIOK3, RNASEL, RPL36A, RRP9, SAT1, SERPINB2, SERPINE1, SGSM2, SIAH2, SIN3A, SIRPA, SLC16A1, SLC16A2, SLC20A1, SLC2A1, SLC2A3, SLC3A2, SLC6A10P, SLC6A16, SLC6A6, SLC6A8, SORL1, SPP1, SRSF6, SSSCA1, STC2, STRA13, SYT7, TBPL1, TCEAL1, TEK, TF, TFF3, TFRC, TGFA, TGFB1, TGFB3, TGFBI, TGM2, TH, THBS1, THBS2, TIMM17A, TNFAIP3, TP53, TPBG, TPD52, TPI1, TXN, TXNIP, UMPS, VEGFA, VEGFB, VEGFC, VIM, VPS11, and XRCC6. In some embodiments, the GeneSetScore (Up hypoxia) is determined using RNA-seq, for example, single-cell RNA-seq (scRNA-seq), for example, as exemplified in Example 10 with respect to FIG. 39D. In some embodiments, the GeneSetScore (Up hypoxia) is calculated by taking the mean log normalized gene expression value of all of the genes in the gene set.

As used herein, the term “GeneSetScore (Up autophagy)” of a cell refers to a score that reflects the degree at which the cell shows an autophagy phenotype. A higher GeneSetScore (Up autophagy) indicates an increasing autophagy phenotype. In some embodiments, the GeneSetScore (Up autophagy) is determined by measuring the expression of one or more genes that are up-regulated in cells undergoing autophagy, for example, one or more genes selected from the group consisting of ABL1, ACBD5, ACIN1, ACTRT1, ADAMTS7, AKR1E2, ALKBH5, ALPK1, AMBRA1, ANXA5, ANXA7, ARSB, ASB2, ATG10, ATG12, ATG13, ATG14, ATG16L1, ATG16L2, ATG2A, ATG2B, ATG3, ATG4A, ATG4B, ATG4C, ATG4D, ATG5, ATG7, ATG9A, ATG9B, ATP13A2, ATP1B1, ATPAF1-AS1, ATPIF1, BECN1, BECN1P1, BLOC1S1, BMP2KL, BNIP1, BNIP3, BOC, C11orf2, C11orf41, C12orf44, C12orf5, C14orf133, C1orf210, C5, C6orf106, C7orf59, C7orf68, C8orf59, C9orf72, CA7, CALCB, CALCOCO2, CAPS, CCDC36, CD163L1, CD93, CDC37, CDKN2A, CHAF1B, CHMP2A, CHMP2B, CHMP3, CHMP4A, CHMP4B, CHMP4C, CHMP6, CHST3, CISD2, CLDN7, CLEC16A, CLN3, CLVS1, COX8A, CPA3, CRNKL1, CSPG5, CTSA, CTSB, CTSD, CXCR7, DAP, DKKL1, DNAAF2, DPF3, DRAM1, DRAM2, DYNLL1, DYNLL2, DZANK1, EI24, EIF2S1, EPG5, EPM2A, FABP1, FAM125A, FAM131B, FAM134B, FAM13B, FAM176A, FAM176B, FAM48A, FANCC, FANCF, FANCL, FBXO7, FCGR3B, FGF14, FGF7, FGFBP1, FIS1, FNBP1L, FOXO1, FUNDC1, FUNDC2, FXR2, GABARAP, GABARAPL1, GABARAPL2, GABARAPL3, GABRA5, GDF5, GMIP, HAP1, HAPLN1, HBXIP, HCAR1, HDAC6, HGS, HIST1H3A, HIST1H3B, HIST1H3C, HIST1H3D, HIST1H3E, HIST1H3F, HIST1H3G, HIST1H3H, HIST1H3I, HIST1H3J, HK2, HMGB1, HPR, HSF2BP, HSP90AA1, HSPA8, IFI16, IPPK, IRGM, IST1, ITGB4, ITPKC, KCNK3, KCNQ1, KIAA0226, KIAA1324, KRCC1, KRT15, KRT73, LAMP1, LAMP2, LAMTOR1, LAMTOR2, LAMTOR3, LARP1B, LENG9, LGALS8, LIX1, LIX1L, LMCD1, LRRK2, LRSAM1, LSM4, MAP1A, MAP1LC3A, MAP1LC3B, MAP1LC3B2, MAP1LC3C, MAP1S, MAP2K1, MAP3K12, MARK2, MBD5, MDH1, MEX3C, MFN1, MFN2, MLST8, MRPS10, MRPS2, MSTN, MTERFD1, MTMR14, MTMR3, MTOR, MTSS1, MYH11, MYLK, MYOM1, NBR1, NDUFB9, NEFM, NHLRC1, NME2, NPC1, NR2C2, NRBF2, NTHL1, NUP93, OBSCN, OPTN, P2RX5, PACS2, PARK2, PARK7, PDK1, PDK4, PEX13, PEX3, PFKP, PGK2, PHF23, PHYHIP, PI4K2A, PIK3C3, PIK3CA, PIK3CB, PIK3R4, PINK1, PLEKHM1, PLOD2, PNPO, PPARGC1A, PPY, PRKAA1, PRKAA2, PRKAB1, PRKAB2, PRKAG1, PRKAG2, PRKAG3, PRKD2, PRKG1, PSEN1, PTPN22, RAB12, RAB1A, RAB1B, RAB23, RAB24, RAB33B, RAB39, RAB7A, RB1CC1, RBM18, REEP2, REP15, RFWD3, RGS19, RHEB, RIMS3, RNF185, RNF41, RPS27A, RPTOR, RRAGA, RRAGB, RRAGC, RRAGD, S100A8, S100A9, SCN1A, SERPINB10, SESN2, SFRP4, SH3GLB1, SIRT2, SLC1A3, SLC1A4, SLC22A3, SLC25A19, SLC35B3, SLC35C1, SLC37A4, SLC6A1, SLCO1A2, SMURF1, SNAP29, SNAPIN, SNF8, SNRPB, SNRPB2, SNRPD1, SNRPF, SNTG1, SNX14, SPATA18, SQSTM1, SRPX, STAM, STAM2, STAT2, STBD1, STK11, STK32A, STOM, STX12, STX17, SUPT3H, TBC1D17, TBC1D25, TBC1D5, TCIRG1, TEAD4, TECPR1, TECPR2, TFEB, TM9SF1, TMBIM6, TMEM203, TMEM208, TMEM39A, TMEM39B, TMEM59, TMEM74, TMEM93, TNIK, TOLLIP, TOMM20, TOMM22, TOMM40, TOMM5, TOMM6, TOMM7, TOMM70A, TP53INP1, TP53INP2, TRAPPC8, TREM1, TRIM17, TRIM5, TSG101, TXLNA, UBA52, UBB, UBC, UBQLN1, UBQLN2, UBQLN4, ULK1, ULK2, ULK3, USP10, USP13, USP30, UVRAG, VAMP7, VAMP5, VDAC1, VMP1, VPS11, VPS16, VPS18, VPS25, VPS28, VPS33A, VPS33B, VPS36, VPS37A, VPS37B, VPS37C, VPS37D, VPS39, VPS41, VPS4A, VPS4B, VTA1, VTI1A, VTI1B, WDFY3, WDR45, WDR45L, WIPI1, WIPI2, XBP1, YIPF1, ZCCHC17, ZFYVE1, ZKSCAN3, ZNF189, ZNF593, and ZNF681. In some embodiments, the GeneSetScore (Up autophagy) is determined using RNA-seq, for example, single-cell RNA-seq (scRNA-seq), for example, as exemplified in Example 10 with respect to FIG. 39E. In some embodiments, the GeneSetScore (Up autophagy) is calculated by taking the mean log normalized gene expression value of all of the genes in the gene set.

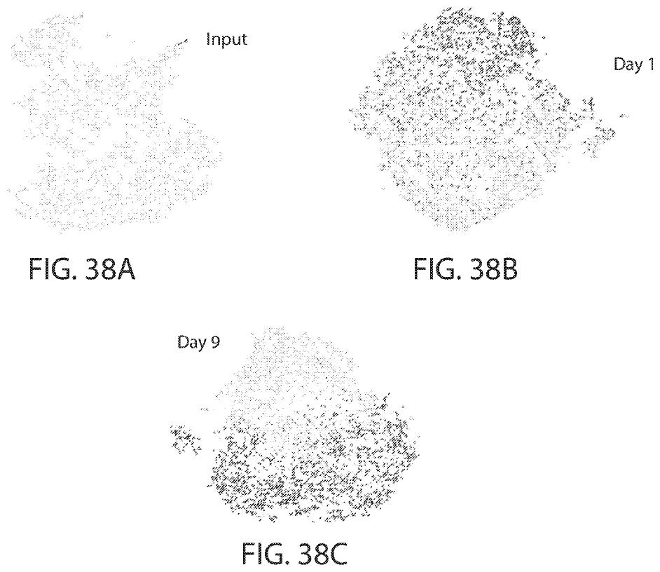

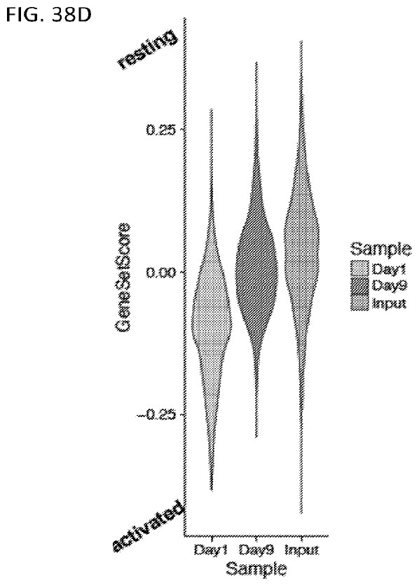

As used herein, the term “GeneSetScore (Up resting vs. Down activated)” of a cell refers to a score that reflects the degree at which the cell shows a resting T cell phenotype vs. an activated T cell phenotype. A higher GeneSetScore (Up resting vs. Down activated) indicates an increasing resting T cell phenotype, whereas a lower GeneSetScore (Up resting vs. Down activated) indicates an increasing activated T cell phenotype. In some embodiments, the GeneSetScore (Up resting vs. Down activated) is determined by measuring the expression of one or more genes that are up-regulated in resting T cells and/or down-regulated in activated T cells, for example, one or more genes selected from the group consisting of ABCA7, ABCF3, ACAP2, AMT, ANKH, ATF7IP2, ATG14, ATP1A1, ATXN7, ATXN7L3B, BCL7A, BEX4, BSDC1, BTG1, BTG2, BTN3A1, C11orf2l, C19orf22, C21orf2, CAMK2G, CARS2, CCNL2, CD248, CD5, CD55, CEP164, CHKB, CLK1, CLK4, CTSL1, DBP, DCUN1D2, DENND1C, DGKD, DLG1, DUSP1, EAPP, ECE1, ECHDC2, ERBB2IP, FAM117A, FAM134B, FAM134C, FAM169A, FAM190B, FAU, FLJ10038, FOXJ2, FOXJ3, FOXL1, FOXO1, FXYD5, FYB, HLA-E, HSPA1L, HYAL2, ICAM2, IFIT5, IFITM1, IKBKB, IQSEC1, IRS4, KIAA0664L3, KIAA0748, KLF3, KLF9, KRT18, LEF1, LINC00342, LIPA, LIPT1, LLGL2, LMBR1L, LPAR2, LTBP3, LYPD3, LZTFL1, MANBA, MAP2K6, MAP3K1, MARCH8, MAU2, MGEA5, MMP8, MPO, MSL1, MSL3, MYH3, MYLIP, NAGPA, NDST2, NISCH, NKTR, NLRP1, NOSIP, NPIP, NUMA1, PAIP2B, PAPD7, PBXIP1, PCIF1, PI4KA, PLCL2, PLEKHAL PLEKHF2, PNISR, PPFIBP2, PRKCA, PRKCZ, PRKD3, PRMT2, PTP4A3, PXN, RASA2, RASA3, RASGRP2, RBM38, REPIN1, RNF38, RNF44, ROR1, RPL30, RPL32, RPLP1, RPS20, RPS24, RPS27, RPS6, RPS9, RXRA, RYK, SCAND2, SEMA4C, SETD1B, SETD6, SETX, SF3B1, SH2B1, SLC2A4RG, SLC35E2B, SLC46A3, SMAGP, SMARCE1, SMPD1, SNPH, SP140L, SPATA6, SPG7, SREK1IP1, SRSF5, STAT5B, SVIL, SYF2, SYNJ2BP, TAF1C, TBC1D4, TCF20, TECTA, TES, TMEM127, TMEM159, TMEM30B, TMEM66, TMEM8B, TP53TG1, TPCN1, TRIM22, TRIM44, TSC1, TSC22D1, TSC22D3, TSPYL2, TTC9, TTN, UBE2G2, USP33, USP34, VAMP1, VILL, VIPR1, VPS13C, ZBED5, ZBTB25, ZBTB40, ZC3H3, ZFP161, ZFP36L1, ZFP36L2, ZHX2, ZMYMS, ZNF136, ZNF148, ZNF318, ZNF350, ZNF512B, ZNF609, ZNF652, ZNF83, ZNF862, and ZNF91. In some embodiments, the GeneSetScore (Up resting vs. Down activated) is determined using RNA-seq, for example, single-cell RNA-seq (scRNA-seq), for example, as exemplified in Example 10 with respect to FIG. 38D. In some embodiments, the GeneSetScore (Up resting vs. Down activated) is calculated by taking the mean log normalized gene expression value of all of the genes in the gene set.

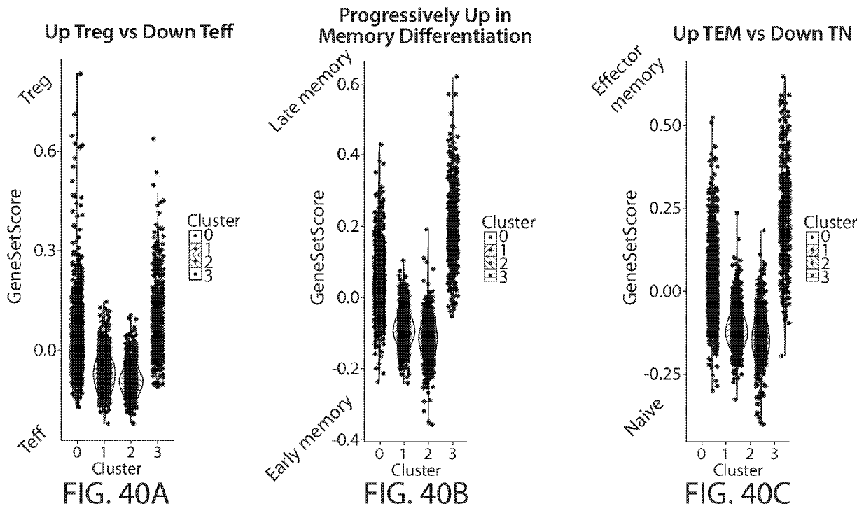

As used herein, the term “GeneSetScore (Progressively up in memory differentiation)” of a cell refers to a score that reflects the stage of the cell in memory differentiation. A higher GeneSetScore (Progressively up in memory differentiation) indicates an increasing late memory T cell phenotype, whereas a lower GeneSetScore (Progressively up in memory differentiation) indicates an increasing early memory T cell phenotype. In some embodiments, the GeneSetScore (Up autophagy) is determined by measuring the expression of one or more genes that are up-regulated during memory differentiation, for example, one or more genes selected from the group consisting of MTCH2, RAB6C, KIAA0195, SETD2, C2orf24, NRD1, GNA13, COPA, SELT, TNIP1, CBFA2T2, LRP10, PRKCI, BRE, ANKS1A, PNPLA6, ARL6IP1, WDFY1, MAPK1, GPR153, SHKBP1, MAP1LC3B2, PIP4K2A, HCN3, GTPBP1, TLN1, C4orf34, KIF3B, TCIRG1, PPP3CA, ATG4D, TYMP, TRAF6, C17orf76, WIPF1, FAM108A1, MYL6, NRM, SPCS2, GGT3P, GALK1, CLIP4, ARL4C, YWHAQ, LPCAT4, ATG2A, IDS, TBC1D5, DMPK, ST6GALNAC6, REEP5, ABHD6, KIAA0247, EMB, TSEN54, SPIRE2, PIWIL4, ZSCAN22, ICAM1, CHD9, LPIN2, SETD8, ZC3H12A, ULBP3, IL15RA, HLA-DQB2, LCP1, CHP, RUNX3, TMEM43, REEP4, MEF2D, ABL1, TMEM39A, PCBP4, PLCD1, CHST12, RASGRP1, C1orf58, C11orf63, C6orf129, FHOD1, DKFZp434F142, PIK3CG, ITPR3, BTG3, C4orf50, CNNM3, IFI16, AK1, CDK2AP1, REL, BCL2L1, MVD, TTC39C, PLEKHA2, FKBP11, EML4, FANCA, CDCA4, FUCA2, MFSD10, TBCD, CAPN2, IQGAP1, CHST11, PIK3R1, MYO5A, KIR2DL3, DLG3, MXD4, RALGDS, S1PR5, WSB2, CCR3, TIPARP, SP140, CD151, SOX13, KRTAP5-2, NF1, PEA15, PARP8, RNF166, UEVLD, LIMK1, CACNB1, TMX4, SLC6A6, LBA1, SV2A, LLGL2, IRF1, PPP2R5C, CD99, RAPGEF1, PPP4R1, OSBPL7, FOXP4, SLA2, TBC1D2B, ST7, JAZF1, GGA2, PI4K2A, CD68, LPGAT1, STX11, ZAK, FAM160B1, RORA, C8orf80, APOBEC3F, TGFBI, DNAJC1, GPR114, LRP8, CD69, CMIP, NAT13, TGFB1, FLJ00049, ANTXR2, NR4A3, IL12RB1, NTNG2, RDX, MLLT4, GPRIN3, ADCY9, CD300A, SCD5, ABI3, PTPN22, LGALS1, SYTL3, BMPR1A, TBK1, PMAIP1, RASGEF1A, GCNT1, GABARAPL1, STOM, CALHM2, ABCA2, PPP1R16B, SYNE2, PAM, C12orf75, CLCF1, MXRA7, APOBEC3C, CLSTN3, ACOT9, HIP1, LAG3, TNFAIP3, DCBLD1, KLF6, CACNB3, RNF19A, RAB27A, FADS3, DLG5, APOBEC3D, TNFRSF1B, ACTN4, TBKBP1, ATXN1, ARAP2, ARHGEF12, FAM53B, MAN1A1, FAM38A, PLXNC1, GRLF1, SRGN, HLA-DRBS, B4GALT5, WIPI1, PTPRJ, SLFN11, DUSP2, ANXA5, AHNAK, NEO1, CLIC1, EIF2C4, MAP3K5, IL2RB, PLEKHG1, MYO6, GTDC1, EDARADD, GALM, TARP, ADAMS, MSC, HNRPLL, SYT11, ATP2B4, NHSL2, MATK, ARHGAP18, SLFN12L, SPATS2L, RAB27B, PIK3R3, TP53INP1, MBOAT1, GYG1, KATNAL1, FAM46C, ZC3HAV1L, ANXA2P2, CTNNA1, NPC1, C3AR1, CRIM1, SH2D2A, ERN1, YPEL1, TBX21, SLC1A4, FASLG, PHACTR2, GALNT3, ADRB2, PIK3AP1, TLR3, PLEKHAS, DUSP10, GNAO1, PTGDR, FRMD4B, ANXA2, EOMES, CADM1, MAF, TPRG1, NBEAL2, PPP2R2B, PELO, SLC4A4, KLRF1, FOSL2, RGS2, TGFBR3, PRF1, MYO1F, GAB3, C17orf66, MICAL2, CYTH3, TOX, HLA-DRA, SYNE1, WEE1, PYHIN1, F2R, PLD1, THBS1, CD58, FAS, NETO2, CXCR6, ST6GALNAC2, DUSP4, AUTS2, C1orf2l, KLRG1, TNIP3, GZMA, PRR5L, PRDM1, ST8SIA6, PLXND1, PTPRM, GFPT2, MYBL1, SLAMF7, FLJ16686, GNLY, ZEB2, CST7, IL18RAP, CCL5, KLRD1, and KLRB1. In some embodiments, the GeneSetScore (Progressively up in memory differentiation) is determined using RNA-seq, for example, single-cell RNA-seq (scRNA-seq), for example, as exemplified in Example 10 with respect to FIG. 40B. In some embodiments, the GeneSetScore (Progressively up in memory differentiation) is calculated by taking the mean log normalized gene expression value of all of the genes in the gene set.

As used herein, the term “GeneSetScore (Up TEM vs. Down TN)” of a cell refers to a score that reflects the degree at which the cell shows an effector memory T cell (TEM) phenotype vs. a naïve T cell (TN) phenotype. A higher GeneSetScore (Up TEM vs. Down TN) indicates an increasing TEM phenotype, whereas a lower GeneSetScore (Up TEM vs. Down TN) indicates an increasing TN phenotype. In some embodiments, the GeneSetScore (Up TEM vs. Down TN) is determined by measuring the expression of one or more genes that are up-regulated in TEM cells and/or down-regulated in TN cells, for example, one or more genes selected from the group consisting of MYO5A, MXD4, STK3, S1PR5, GLCCI1, CCR3, SOX13, KRTAP5-2, PEA15, PARP8, RNF166, UEVLD, LIMK1, SLC6A6, SV2A, KPNA2, OSBPL7, ST7, GGA2, PI4K2A, CD68, ZAK, RORA, TGFBI, DNAJC1, JOSD1, ZFYVE28, LRP8, OSBPL3, CMIP, NAT13, TGFB1, ANTXR2, NR4A3, RDX, ADCY9, CHN1, CD300A, SCD5, PTPN22, LGALS1, RASGEF1A, GCNT1, GLUL, ABCA2, CLDND1, PAM, CLCF1, MXRA7, CLSTN3, ACOT9, METRNL, BMPR1A, LRIG1, APOBEC3G, CACNB3, RNF19A, RAB27A, FADS3, ACTN4, TBKBP1, FAM53B, MAN1A1, FAM38A, GRLF1, B4GALT5, WIPI1, DUSP2, ANXA5, AHNAK, CLIC1, MAP3K5, ST8SIA1, TARP, ADAMS, MATK, SLFN12L, PIK3R3, FAM46C, ANXA2P2, CTNNA1, NPC1, SH2D2A, ERN1, YPEL1, TBX21, STOM, PHACTR2, GBP5, ADRB2, PIK3AP1, DUSP10, PTGDR, EOMES, MAF, TPRG1, NBEAL2, NCAPH, SLC4A4, FOSL2, RGS2, TGFBR3, MYO1F, C17orf66, CYTH3, WEE1, PYHIN1, F2R, THBS1, CD58, AUTS2, FAM129A, TNIP3, GZMA, PRR5L, PRDM1, PLXND1, PTPRM, GFPT2, MYBL1, SLAMF7, ZEB2, CST7, CCL5, GZMK, and KLRB1. In some embodiments, the GeneSetScore (Up TEM vs. Down TN) is determined using RNA-seq, for example, single-cell RNA-seq (scRNA-seq), for example, as exemplified in Example 10 with respect to FIG. 40C. In some embodiments, the GeneSetScore (Up TEM vs. Down TN) is calculated by taking the mean log normalized gene expression value of all of the genes in the gene set.

In the context of GeneSetScore values (e.g., median GeneSetScore values), when a positive GeneSetScore is reduced by 100%, the value becomes 0. When a negative GeneSetScore is increased by 100%, the value becomes 0. For example, in FIG. 39A, the median GeneSetScore of the Dayl sample is −0.084; the median GeneSetScore of the Day9 sample is 0.035; and the median GeneSetScore of the input sample is −0.1. In FIG. 39A, increasing the median GeneSetScore of the input sample by 100% leads to a GeneSetScore value of 0; and increasing the median GeneSetScore of the input sample by 200% leads to a GeneSetScore value of 0.1. In FIG. 39A, decreasing the median GeneSetScore of the Day9 sample by 100% leads to a GeneSetScore value of 0; and decreasing the median GeneSetScore of the Day9 sample by 200% leads to a GeneSetScore value of −0.035.

As used herein, the term “bead” refers to a discrete particle with a solid surface, ranging in size from approximately 0.1 μm to several millimeters in diameter. Beads may be spherical (for example, microspheres) or have an irregular shape. Beads may comprise a variety of materials including, but not limited to, paramagnetic materials, ceramic, plastic, glass, polystyrene, methylstyrene, acrylic polymers, titanium, latex, Sepharose™, cellulose, nylon and the like. In some embodiments, the beads are relatively uniform, about 4.5 μm in diameter, spherical, superparamagnetic polystyrene beads, for example, coated, for example, covalently coupled, with a mixture of antibodies against CD3 (for example, CD3 epsilon) and CD28. In some embodiments, the beads are Dynabeads®. In some embodiments, both anti-CD3 and anti-CD28 antibodies are coupled to the same bead, mimicking stimulation of T cells by antigen presenting cells. The property of Dynabeads® and the use of Dynabeads® for cell isolation and expansion are well known in the art, for example, see, Neurauter et al., Cell isolation and expansion using Dynabeads, Adv Biochem Eng Biotechnol. 2007; 106:41-73, herein incorporated by reference in its entirety.

As used herein, the term “nanomatrix” refers to a nanostructure comprising a matrix of mobile polymer chains. The nanomatrix is 1 to 500 nm, for example, 10 to 200 nm, in size. In some embodiments, the matrix of mobile polymer chains is attached to one or more agonists which provide activation signals to T cells, for example, agonist anti-CD3 and/or anti-CD28 antibodies. In some embodiments, the nanomatrix comprises a colloidal polymeric nanomatrix attached, for example, covalently attached, to an agonist of one or more stimulatory molecules and/or an agonist of one or more costimulatory molecules. In some embodiments, the agonist of one or more stimulatory molecules is a CD3 agonist (for example, an anti-CD3 agonistic antibody). In some embodiments, the agonist of one or more costimulatory molecules is a CD28 agonist (for example, an anti-CD28 agonistic antibody). In some embodiments, the nanomatrix is characterized by the absence of a solid surface, for example, as the attachment point for the agonists, such as anti-CD3 and/or anti-CD28 antibodies. In some embodiments, the nanomatrix is the nanomatrix disclosed in WO2014/048920A1 or as given in the MACS® GMP T Cell TransAct™ kit from Miltenyi Biotcc GmbH, herein incorporated by reference in their entirety. MACS® GMP T Cell TransAct™ consists of a colloidal polymeric nanomatrix covalently attached to humanized recombinant agonist antibodies against human CD3 and CD28.

As used herein, “ubiquitination” refers to the addition of a ubiquitin molecule, e.g., a single ubiquitin (mono-ubiquitination) or more than one ubiquitin (e.g., a chain of ubiquitin molecules, or poly-ubiquitination). Ubiquitination can be performed by an enzyme machinery including one or more of a ubiquitin-activating enzyme (E1), a ubiquitin-conjugating enzyme (E2), and a ubiquitin ligase (E3).

As used herein, the term “CRBN” refers to a protein that in humans is encoded by the CRBN gene, or fragment or variant thereof (e.g., an amino acid sequence substantially identical thereto, e.g., 30 least 85, 87, 90, 95, 97, 98, 99, or 100% identical thereto). Swiss-Prot accession number Q96SW2 provides exemplary human CRBN amino acid sequences.

As used herein, an “IKZF polypeptide” refers to an IKZF, or fragment or variant thereof (e.g., an amino acid sequence substantially identical thereto, e.g., least 85, 87, 90, 95, 97, 98, 99, or 100% identical thereto).

As used herein, the term “IKZF3” refers to a protein that in humans is encoded by the IKZF3 gene. Swiss-Prot accession number Q9UKT9 provides exemplary human IKZF3 amino acid sequences. An exemplary human IKZF3 amino acid sequence is provided in SEQ ID NO: 328. The term “IKZF3 polypeptide” refers to IKZF3, or fragment or variant thereof (e.g., an amino acid sequence substantially identical thereto, e.g., least 85, 87, 90, 95, 97, 98, 99, or 100% identical thereto).

As used herein, the term “IKZF1” refers to a protein that in humans is encoded by the IKZF1 gene. Swiss-Prot accession number Q13422 provides exemplary human IKZF1 amino acid sequences. An exemplary human IKZF1 amino acid sequence is provided in SEQ ID NO: 329. The term “IKZF1 polypeptide” refers to IKZF1, or fragment or variant thereof (e.g., an amino acid sequence substantially identical thereto, e.g., least 85, 87, 90, 95, 97, 98, 99, or 100% identical thereto).

As used herein, the term “IKZF2” refers to a protein that in humans is encoded by the IKZF2 gene. Swiss-Prot accession number Q9UKS7 provides exemplary human IKZF2 amino acid sequences. An exemplary human IKZF2 amino acid sequence is provided in SEQ ID NO: 330. The term “IKZF2 polypeptide” refers to IKZF2, or fragment or variant thereof (e.g., an amino acid sequence substantially identical thereto, e.g., least 85, 87, 90, 95, 97, 98, 99, or 100% identical thereto).

As used herein, the term “IKZF4” refers to a protein that in humans is encoded by the IKZF4 gene. Swiss-Prot accession number Q9H2S9 provides exemplary human IKZF4 amino acid sequences. An exemplary human IKZF4 amino acid sequence is provided in SEQ ID NO: 331. The term “IKZF4 polypeptide” refers to IKZF4, or fragment or variant thereof (e.g., an amino acid sequence substantially identical thereto, e.g., least 85, 87, 90, 95, 97, 98, 99, or 100% identical thereto).

As used herein, the term “IKZF5” refers to a protein that in humans is encoded by the IKZF5 gene. Swiss-Prot accession number Q9H5V7 provides exemplary human IKZF5 amino acid sequences. An exemplary human IKZF5 amino acid sequence is provided in SEQ ID NO: 332. The term “IKZF5 polypeptide” refers to IKZF5, or fragment or variant thereof (e.g., an amino acid sequence substantially identical thereto, e.g., least 85, 87, 90, 95, 97, 98, 99, or 100% identical thereto).

As used herein, a “fusion polypeptide” or “chimeric polypeptide” refers to a polypeptide that includes two or more heterologous amino acid sequences and/or protein domains in a single, continuous polypeptide. In some embodiments, the two or more heterologous protein domains are covalently linked directly or indirectly, e.g., via a linker.

As used herein, the term “estrogen receptor (ER)” refers to a protein that in humans is encoded by the ESR1 gene. Swiss-Prot accession number P03372 provides exemplary human estrogen receptor (ER) amino acid sequences. An “estrogen receptor (ER) domain” refers to estrogen receptor, or fragment or variant thereof (e.g., an amino acid sequence substantially identical thereto, e.g., least 85, 87, 90, 95, 97, 98, 99, or 100% identical thereto). Exemplary estrogen receptor (ER) domain amino acid sequences are provided in SEQ ID NOs: 340, 342 and 344. Exemplary estrogen receptor (ER) domain nucleotide sequences are provided in SEQ ID NOs: 341, 343 and 345.

As used herein, an “FKB protein (FKBP) domain” refers to FKBP, or fragment or variant thereof. An exemplary FKB protein (FKBP) domain amino acid sequence is provided in SEQ ID NO: 346.

As used herein, the term “dihydrofolate reductase (DHFR)” refers to a protein that in humans is encoded by the DHFR gene. Swiss-Prot accession number P00374 provides exemplary human dihydrofolate reductase (DHFR) amino acid sequences. A “dihydrofolate reductase (DHFR) domain” refers to DHFR, or fragment or variant thereof. An exemplary dihydrofolate reductase (DHFR) domain amino acid sequence is provided in SEQ ID NO: 347.

As used herein, the term “degradation domain” refers to a domain of a fusion polypeptide that assumes a stable conformation when expressed in the presence of a stabilization compound. Absent the stable conformation when expressed in a cell of interest, a large fraction of degradation domains (and, typically, any protein to which they are fused to) will be degraded by endogenous cellular machinery. Notably, a degradation domain is not a naturally occurring domain of a protein but is rather engineered to be unstable absent contact with the stabilization compound. Thus, a degradation domain is identifiable by the following characteristics: (1) it is not naturally occurring; (2) its expression is regulated co-translationally or post-translationally through increased or decreased degradation rates; (3) the rate of degradation is substantially decreased in the presence of a stabilization compound. In some embodiments, absent a stabilization compound, the degradation domain or other domain of the fusion polypeptide is not substantially detectable in or on the cell. In some embodiments, the degradation domain is in a destabilized state in the absence of a stabilization compound. In some embodiments, the degradation domain does not self-associate, e.g., does not homodimerize, in the absence of a stabilization compound. In some embodiments, the degradation domain is fused to a heterologous protease cleavage site, wherein in the presence of the stabilization compound, the cleavage of the heterologous protease cleavage site is more efficient than in the absence of the stabilization compound.

The degradation domain is not an aggregation domain as defined in PCT Application Number PCT/US2017/027778.

By “stabilization compound” or “stabilizing compound” is meant a compound that, when added to a cell expressing a degradation domain, stabilizes the degradation domain and any protein that is fused to it, and decreases the rate at which it is subsequently degraded. Stabilization compounds or stabilizing compounds can be naturally occurring or synthetic.

Furthermore, by “heterologous protease cleavage site” is meant a protease cleavage site that has a different origin than one or more protein domains to which it is fused (e.g., is not naturally fused to at least one of the other referenced domains)

By “protease” is meant a protein that cleaves another protein based on the presence of a cleavage site in the to-be-cleaved protein.

By “intracellular protease” is meant a protease that is natively expressed inside a cell of interest.

By “extracellular protease” is meant a protease that is natively expressed in an organism (e.g., a mammal) and secreted or exposed to the outside of cells (e.g., in the blood or the surface of the skin).

As used herein, the term “cleavage” refers to the breakage of covalent bonds, such as in the backbone of a nucleic acid molecule or the hydrolysis of peptide bonds. Cleavage can be initiated by a variety of methods, including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The term “alkyl,” as used herein, refers to a monovalent saturated, straight- or branched-chain hydrocarbon such as a straight or branched group of 1-12, 1-10, or 1-6 carbon atoms, referred to herein as C₁-C₁₂ alkyl, C₁-C₁₀ alkyl, and C₁-C₆ alkyl, respectively. Examples of alkyl groups include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, and the like.

The terms “alkenyl” and “alkynyl” as used herein refer to unsaturated aliphatic groups analogous in length and possible substitution to the alkyls described above, but that contain at least one double or triple bond, respectively. Exemplary alkenyl groups include, but are not limited to, —CH═CH₂ and —CH₂CH═CH₂.

The term “alkoxy” as used herein refers to a straight or branched chain saturated hydrocarbon containing 1-12 carbon atoms containing a terminal “O” in the chain, e.g., —O(alkyl). Examples of alkoxy groups include, without limitation, methoxy, ethoxy, propoxy, butoxy, t-butoxy, or pentoxy groups.

The term “aryl” as used herein refers to a monocyclic, bicyclic or polycyclic hydrocarbon ring system, wherein at least one ring is aromatic. Representative aryl groups include fully aromatic ring systems, such as phenyl (e.g., (C₆) aryl), naphthyl (e.g., (C₁₀) aryl), and anthracenyl (e.g., (C₁₄) aryl), and ring systems where an aromatic carbon ring is fused to one or more non-aromatic carbon rings, such as indanyl, phthalimidyl, naphthimidyl, or tetrahydronaphthyl, and the like.

The term “carbocyclyl” as used herein refers to monocyclic, or fused, spiro-fused, and/or bridged bicyclic or polycyclic hydrocarbon ring system containing 3-18 carbon atoms, wherein each ring is either completely saturated or contains one or more units of unsaturation, but where no ring is aromatic. Representative carbocyclyl groups include cycloalkyl groups (e.g., cyclopentyl, cyclobutyl, cyclopentyl, cyclohexyl and the like), and cycloalkenyl groups (e.g., cyclopentenyl, cyclohexenyl, cyclopentadienyl, and the like).

The term “carbonyl” as used herein refers to —C═O.

The term “cyano” as used herein refers to —CN.

The terms “halo” or “halogen” as used herein refer to fluorine (fluoro, —F), chlorine (chloro, —Cl), bromine (bromo, —Br), or iodine (iodo, —I).

The term “haloalkyl” as used herein refers to a monovalent saturated straight or branched alkyl chain wherein at least one carbon atom in the chain is substituted with one or more halogen atoms. In some embodiments, a haloalkyl group may comprise, e.g., 1-12, 1-10, or 1-6 carbon atoms, referred to herein as C₁-C₁₂ haloalkyl, C₁-C₁₀ haloalkyl, and C₁-C₆haloalkyl. Examples of haloalkyl groups include, but are not limited to, trifluoromethyl, difluoromethyl, pentafluoroethyl, trichloromethyl, etc.

The term “haloalkoxy” to a straight or branched chain saturated hydrocarbon containing 1-12 carbon atoms containing a terminal “O” in the chain, wherein at least one carbon atom in the chain is substituted with one or more halogens. Examples of haloalkoxy groups include, but are not limited to, trifluoromethoxy, difluoromethoxy, pentafluoroethoxy, trichloromethoxy, etc.

The term “heteroalkyl” as used herein refers to a monovalent saturated straight or branched alkyl chain wherein at least one carbon atom in the chain is replaced with a heteroatom, such as O, S, or N, provided that upon substitution, the chain comprises at least one carbon atom. In some embodiments, a heteroalkyl group may comprise, e.g., 1-12, 1-10, or 1-6 carbon atoms, referred to herein as C₁-C₁₂ heteroalkyl, C₁-C₁₀ heteroalkyl, and C₁-C₆ heteroalkyl. In certain instances, a heteroalkyl group comprises 1, 2, 3, or 4 independently selected heteroatoms in place of 1, 2, 3, or 4 individual carbon atoms in the alkyl chain. Representative heteroalkyl groups include —CH₂NHC(O)CH₃, —CH₂CH₂OCH₃, —CH₂CH₂NHCH₃, —CH₂CH₂N(CH₃)CH₃, and the like.

The terms “alkylene,” “alkenylene”, “alkynylene,” and “heteroalkylene” as used herein refer to a divalent radical of an alkyl, alkenyl, alkynyl, or heteroalkyl group, respectively. Any of a monovalent alkyl, alkenyl, alkynyl, or heteroalkyl group may be an alkylene, alkenylene, alkynylene, or heteroalkylene by abstraction of a second hydrogen atom from the alkyl, alkenyl, alkynyl, or heteroalkyl group.

The term “heteroaryl” as used herein refers to a monocyclic, bicyclic or polycyclic ring system wherein at least one ring is both aromatic and comprises a heteroatom; and wherein no other rings are heterocyclyl (as defined below). Representative heteroaryl groups include ring systems where (i) each ring comprises a heteroatom and is aromatic, e.g., imidazolyl, oxazolyl, thiazolyl, triazolyl, pyrrolyl, furanyl, thiophenyl pyrazolyl, pyridinyl, pyrazinyl, pyridazinyl, pyrimidinyl, indolizinyl, purinyl, naphthyridinyl, and pteridinyl; (ii) each ring is aromatic or carbocyclyl, at least one aromatic ring comprises a heteroatom and at least one other ring is a hydrocarbon ring or e.g., indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, carbazolyl, acridinyl, phenazinyl, phenothiazinyl, phenoxazinyl, pyrido[2,3-b]-1,4-oxazin-3(4H)-one, thiazolo[4,5-c]-pyridinyl, 4,5,6,7-tetrahydrothieno[2,3-c]pyridinyl, 5,6-dihydro-4H-thieno[2,3-c]pyrrolyl, 4,5,6,7,8-tetrahydroquinolinyl and 5,6,7,8-tetrahydroisoquinolinyl; and (iii) each ring is aromatic or carbocyclyl, and at least one aromatic ring shares a bridgehead heteroatom with another aromatic ring, e.g., 4H-quinolizinyl. In certain embodiments, the heteroaryl is a monocyclic or bicyclic ring, wherein each of said rings contains 5 or 6 ring atoms where 1, 2, 3, or 4 of said ring atoms are a heteroatom independently selected from N, O, and S.

The term “heterocyclyl” as used herein refers to a monocyclic, or fused, spiro-fused, and/or bridged bicyclic and polycyclic ring systems where at least one ring is saturated or partially unsaturated (but not aromatic) and comprises a heteroatom. A heterocyclyl can be attached to its pendant group at any heteroatom or carbon atom that results in a stable structure and any of the ring atoms can be optionally substituted. Representative heterocyclyls include ring systems in which (i) every ring is non-aromatic and at least one ring comprises a heteroatom, e.g., tetrahydrofuranyl, tetrahydrothienyl, pyrrolidinyl, pyrrolidonyl, piperidinyl, pyrrolinyl, decahydroquinolinyl, oxazolidinyl, piperazinyl, dioxanyl, dioxolanyl, diazepinyl, oxazepinyl, thiazepinyl, morpholinyl, and quinuclidinyl; (ii) at least one ring is non-aromatic and comprises a heteroatom and at least one other ring is an aromatic carbon ring, e.g., 1,2,3,4-tetrahydroquinolinyl; and (iii) at least one ring is non-aromatic and comprises a heteroatom and at least one other ring is aromatic and comprises a heteroatom, e.g., 3,4-dihydro-1H-pyrano[4,3-c]pyridinyl, and 1,2,3,4-tetrahydro-2,6-naphthyridinyl. In certain embodiments, the heterocyclyl is a monocyclic or bicyclic ring, wherein each of said rings contains 3-7 ring atoms where 1, 2, 3, or 4 of said ring atoms are a heteroatom independently selected from N, O, and S.

As described herein, compounds of this disclosure may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under this disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

The term “oxo” as used herein refers to ═O.

The term “thiocarbonyl” as used herein refers to C═S.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formula (I), Formula (I-a), and/or Formula (II) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R.x H₂O, wherein R is the compound and wherein x is a number greater than 0. A given compound may form more than one type of hydrates, including, e.g., monohydrates (xis 1), lower hydrates (xis a number greater than 0 and smaller than 1, e.g., hemihydrates (R.0.5 H₂O)), and polyhydrates (x is a number greater than 1, e.g., dihydrates (R.2 H₂O) and hexahydrates (R.6 H₂O)).

It is to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups and a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane that are likewise formed by treatment with acid or base.

Tautomeric forms may be relevant to the attainment of the optimal chemical reactivity and biological activity of a compound of interest.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of this disclosure. Unless otherwise stated, all tautomeric forms of the compounds of this disclosure are within the scope of this disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. In an embodiment, the hydrogen atoms present within any one of the compounds disclosed herein (for example, a compound of Formula (I)) are isotopically enriched in deuterium. Such compounds are useful, for example, as analytical tools, as probes in biological assays, or as therapeutic agents in accordance with the present disclosure.

Where a particular enantiomer is preferred, it may, in some embodiments be provided substantially free of the corresponding enantiomer, and may also be referred to as “optically enriched.” “Optically-enriched,” as used herein, means that the compound is made up of a significantly greater proportion of one enantiomer. In certain embodiments the compound is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques et al., Enantiomers, Racemates and Resolutions (Wiley Interscience, New York, 1981); Wilen, et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972).

Various embodiments of the compositions and methods herein are described in further detail below. Additional definitions are set out throughout the specification.

### Description

Provided herein are methods of manufacturing immune effector cells (for example, T cells or NK cells) engineered to express a CAR, for example, a controllable CAR (CCAR) described herein, compositions comprising such cells, and methods of using such cells for treating a disease, such as cancer, in a subject. In some embodiments, the methods disclosed herein may manufacture immune effector cells engineered to express a CAR in less than 24 hours. Without wishing to be bound by theory, the methods provided herein preserve the undifferentiated phenotype of T cells, such as naïve T cells, during the manufacturing process. These CAR-expressing cells with an undifferentiated phenotype may persist longer and/or expand better in vivo after infusion. In some embodiments, CART cells produced by the manufacturing methods provided herein comprise a higher percentage of stem cell memory T cells, compared to CART cells produced by the traditional manufacturing process, e.g., as measured using scRNA-seq (e.g., as measured using methods described in Example 10 with respect to FIG. 39A). In some embodiments, CART cells produced by the manufacturing methods provided herein comprise a higher percentage of effector T cells, compared to CART cells produced by the traditional manufacturing process, e.g., as measured using scRNA-seq (e.g., as measured using methods described in Example 10 with respect to FIG. 39B). In some embodiments, CART cells produced by the manufacturing methods provided herein better preserve the stemness of T cells, compared to CART cells produced by the traditional manufacturing process, e.g., as measured using scRNA-seq (e.g., as measured using methods described in Example 10 with respect to FIG. 39C). In some embodiments, CART cells produced by the manufacturing methods provided herein show a lower level of hypoxia, compared to CART cells produced by the traditional manufacturing process, e.g., as measured using scRNA-seq (e.g., as measured using methods described in Example 10 with respect to FIG. 39D). In some embodiments, CART cells produced by the manufacturing methods provided herein show a lower level of autophagy, compared to CART cells produced by the traditional manufacturing process, e.g., as measured using scRNA-seq (e.g., as measured using methods described in Example 10 with respect to FIG. 39E).

In some embodiments, the methods disclosed herein do not involve using a bead, such as Dynabeads® (for example, CD3/CD28 Dynabeads®), and do not involve a de-beading step. In some embodiments, the CART cells manufactured by the methods disclosed herein may be administered to a subject with minimal ex vivo expansion, for example, less than 1 day, less than 12 hours, less than 8 hours, less than 6 hours, less than 4 hours, less than 3 hours, less than 2 hours, less than 1 hour, or no ex vivo expansion. Accordingly, the methods described herein provide a fast manufacturing process of making improved CAR-expressing cell products for use in treating a disease in a subject.

### Cytokine Process

In some embodiments, the present disclosure provides methods of making a population of cells (for example, T cells) that express a chimeric antigen receptor (CAR), e.g., a CAR disclosed herein, e.g., a CCAR disclosed herein. In some embodiments, the population of cells further express a regulatory molecule. In some embodiments, the population of cells express a CCAR disclosed herein. In some embodiments, the population of cells express a CAR disclosed herein and a regulatory molecule disclosed herein. In some embodiments, the method comprises: (1) contacting a population of cells with a cytokine chosen from IL-2, IL-7, IL-15, IL-21, IL-6, or a combination thereof, (2) contacting the population of cells (for example, T cells) with a nucleic acid molecule (for example, a DNA or RNA molecule) encoding the CAR, thereby providing a population of cells (for example, T cells) comprising the nucleic acid molecule, and (3) harvesting the population of cells (for example, T cells) for storage (for example, reformulating the population of cells in cryopreservation media) or administration, wherein: (a) step (2) is performed together with step (1) or no later than 5 hours after the beginning of step (1), for example, no later than 1, 2, 3, 4, or 5 hours after the beginning of step (1), and step (3) is performed no later than 26 hours after the beginning of step (1), for example, no later than 22, 23, or 24 hours after the beginning of step (1), for example, no later than 24 hours after the beginning of step (1), or (b) the population of cells from step (3) are not expanded, or expanded by no more than 5, 10, 15, 20, 25, 30, 35, or 40%, for example, no more than 10%, for example, as assessed by the number of living cells, compared to the population of cells at the beginning of step (1). In some embodiments, the nucleic acid molecule in step (2) is a DNA molecule. In some embodiments, the nucleic acid molecule in step (2) is an RNA molecule. In some embodiments, the nucleic acid molecule in step (2) is on a viral vector, for example, a viral vector chosen from a lentivirus vector, an adenoviral vector, or a retrovirus vector. In some embodiments, the nucleic acid molecule in step (2) is on a non-viral vector. In some embodiments, the nucleic acid molecule in step (2) is on a plasmid. In some embodiments, the nucleic acid molecule in step (2) is not on any vector. In some embodiments, step (2) comprises transducing the population of cells (for example, T cells) with a viral vector comprising a nucleic acid molecule encoding the CAR.

In some embodiments, the population of cells (for example, T cells) is collected from an apheresis sample (for example, a leukapheresis sample) from a subject.

In some embodiments, the apheresis sample (for example, a leukapheresis sample) is collected from the subject and shipped as a frozen sample (for example, a cryopreserved sample) to a cell manufacturing facility. The frozen apheresis sample is then thawed, and T cells (for example, CD4+ T cells and/or CD8+ T cells) are selected from the apheresis sample, for example, using a cell sorting machine (for example, a CliniMACS® Prodigy® device). The selected T cells (for example, CD4+ T cells and/or CD8+ T cells) are then seeded for CART manufacturing using the cytokine process described herein. In some embodiments, at the end of the cytokine process, the CAR T cells are cryopreserved and later thawed and administered to the subject. In some embodiments, the selected T cells (for example, CD4+ T cells and/or CD8+ T cells) undergo one or more rounds of freeze-thaw before being seeded for CART manufacturing.

In some embodiments, the apheresis sample (for example, a leukapheresis sample) is collected from the subject and shipped as a fresh product (for example, a product that is not frozen) to a cell manufacturing facility. T cells (for example, CD4+ T cells and/or CD8+ T cells) are selected from the apheresis sample, for example, using a cell sorting machine (for example, a CliniMACS® Prodigy® device). The selected T cells (for example, CD4+ T cells and/or CD8+ T cells) are then seeded for CART manufacturing using the cytokine process described herein. In some embodiments, the selected T cells (for example, CD4+ T cells and/or CD8+ T cells) undergo one or more rounds of freeze-thaw before being seeded for CART manufacturing.

In some embodiments, the apheresis sample (for example, a leukapheresis sample) is collected from the subject. T cells (for example, CD4+ T cells and/or CD8+ T cells) are selected from the apheresis sample, for example, using a cell sorting machine (for example, a CliniMACS® Prodigy® device). The selected T cells (for example, CD4+ T cells and/or CD8+ T cells) are then shipped as a frozen sample (for example, a cryopreserved sample) to a cell manufacturing facility. The selected T cells (for example, CD4+ T cells and/or CD8+ T cells) are later thawed and seeded for CART manufacturing using the cytokine process described herein.

In some embodiments, after cells (for example, T cells) are seeded, one or more cytokines (for example, one or more cytokines chosen from IL-2, IL-7, IL-15 (for example, hetIL-15 (IL15/sIL-15Ra)), IL-21, or IL-6 (for example, IL-6/sIL-6R)) as well as vectors (for example, lentiviral vectors) encoding a CAR are added to the cells. After incubation for 20-24 hours, the cells are washed and formulated for storage or administration.

Different from traditional CART manufacturing approaches, the cytokine process provided herein does not involve CD3 and/or CD28 stimulation, or ex vivo T cell expansion. T cells that are contacted with anti-CD3 and anti-CD28 antibodies and expanded extensively ex vivo tend to show differentiation towards a central memory phenotype. Without wishing to be bound by theory, the cytokine process provided herein preserves or increases the undifferentiated phenotype of T cells during CART manufacturing, generating a CART product that may persist longer after being infused into a subject.

In some embodiments, the population of cells is contacted with one or more cytokines (for example, one or more cytokines chosen from IL-2, IL-7, IL-15 (for example, hetIL-15 (IL15/sIL-15Ra)), IL-21, or IL-6 (for example, IL-6/sIL-6Ra).

In some embodiments, the population of cells is contacted with IL-2. In some embodiments, the population of cells is contacted with IL-7. In some embodiments, the population of cells is contacted with IL-15 (for example, hetIL-15 (IL15/sIL-15Ra)). In some embodiments, the population of cells is contacted with IL-21. In some embodiments, the population of cells is contacted with IL-6 (for example, IL-6/sIL-6Ra). In some embodiments, the population of cells is contacted with IL-2 and IL-7. In some embodiments, the population of cells is contacted with IL-2 and IL-15 (for example, hetIL-15 (IL15/sIL-15Ra)). In some embodiments, the population of cells is contacted with IL-2 and IL-21. In some embodiments, the population of cells is contacted with IL-2 and IL-6 (for example, IL-6/sIL-6Ra). In some embodiments, the population of cells is contacted with IL-7 and IL-15 (for example, hetIL-15 (IL15/sIL-15Ra)). In some embodiments, the population of cells is contacted with IL-7 and IL-21. In some embodiments, the population of cells is contacted with IL-7 and IL-6 (for example, IL-6/sIL-6Ra). In some embodiments, the population of cells is contacted with IL-15 (for example, hetIL-15 (IL15/sIL-15Ra)) and IL-21. In some embodiments, the population of cells is contacted with IL-15 (for example, hetIL-15 (IL15/sIL-15Ra)) and IL-6 (for example, IL-6/sIL-6Ra). In some embodiments, the population of cells is contacted with IL-21 and IL-6 (for example, IL-6/sIL-6Ra). In some embodiments, the population of cells is contacted with IL-7, IL-15 (for example, hetIL-15 (IL15/sIL-15Ra)), and IL-21. In some embodiments, the population of cells is further contacted with a LSD1 inhibitor. In some embodiments, the population of cells is further contacted with a MALT1 inhibitor.

In some embodiments, the population of cells is contacted with 20, 30, 40, 50, 60, 70, 80, 90, 100, 110, 120, 130, 140, 150, 160, 170, 180, 190, 200, 210, 220, 230, 240, 250, 260, 270, 280, 290, or 300 U/ml of IL-2. In some embodiments, the population of cells is contacted with 1, 2, 3, 4, 5, 6, 7, 8, 9, 10, 11, 12, 13, 14, 15, 16, 17, 18, 19, or 20 ng/ml of IL-7. In some embodiments, the population of cells is contacted with 1, 2, 3, 4, 5, 6, 7, 8, 9, 10, 11, 12, 13, 14, 15, 16, 17, 18, 19, or 20 ng/ml of IL-15.

In some embodiments, the population of cells is contacted with a nucleic acid molecule encoding a CAR. In some embodiments, the population of cells is transduced with a DNA molecule encoding a CAR. In some embodiments, the population of cells is contacted with a nucleic acid molecule encoding a CCAR. In some embodiments, the population of cells is transduced with a DNA molecule encoding a CCAR. In some embodiments, the population of cells is contacted with a nucleic acid molecule encoding a CAR and a regulatory molecule. In some embodiments, the population of cells is transduced with a DNA molecule encoding a CAR and a regulatory molecule.

In some embodiments, contacting the population of cells with the nucleic acid molecule encoding the CAR, e.g., the CCAR occurs simultaneously with contacting the population of cells with the one or more cytokines described above. In some embodiments, contacting the population of cells with the nucleic acid molecule encoding the CAR occurs no later than 1, 1.5, 2, 2.5, 3, 3.5, 4, 4.5, 5, 5.5, 6, 6.5, 7, 7.5, 8, 8.5, 9, 9.5 or 10 hours after the beginning of contacting the population of cells with the one or more cytokines described above. In some embodiments, contacting the population of cells with the nucleic acid molecule encoding the CAR, e.g., the CCAR, occurs no later than 5 hours after the beginning of contacting the population of cells with the one or more cytokines described above. In some embodiments, contacting the population of cells with the nucleic acid molecule encoding the CAR, e.g., the CCAR, occurs no later than 4 hours after the beginning of contacting the population of cells with the one or more cytokines described above. In some embodiments, contacting the population of cells with the nucleic acid molecule encoding the CAR, e.g., the CCAR, occurs no later than 3 hours after the beginning of contacting the population of cells with the one or more cytokines described above. In some embodiments, contacting the population of cells with the nucleic acid molecule encoding the CAR, e.g., the CCAR, occurs no later than 2 hours after the beginning of contacting the population of cells with the one or more cytokines described above. In some embodiments, contacting the population of cells with the nucleic acid molecule encoding the CAR, e.g., the CCAR, occurs no later than 1 hour after the beginning of contacting the population of cells with the one or more cytokines described above.

In some embodiments, the population of cells is harvested for storage or administration.

In some embodiments, the population of cells is harvested for storage or administration no later than 72, 60, 48, 36, 32, 31, 30, 29, 28, 27, 26, 25, 24, 23, 22, 21, 20, 19, or 18 hours after the beginning of contacting the population of cells with the one or more cytokines described above. In some embodiments, the population of cells is harvested for storage or administration no later than 26 hours after the beginning of contacting the population of cells with the one or more cytokines described above. In some embodiments, the population of cells is harvested for storage or administration no later than 25 hours after the beginning of contacting the population of cells with the one or more cytokines described above. In some embodiments, the population of cells is harvested for storage or administration no later than 24 hours after the beginning of contacting the population of cells with the one or more cytokines described above. In some embodiments, the population of cells is harvested for storage or administration no later than 23 hours after the beginning of contacting the population of cells with the one or more cytokines described above. In some embodiments, the population of cells is harvested for storage or administration no later than 22 hours after the beginning of contacting the population of cells with the one or more cytokines described above.

In some embodiments, the population of cells is not expanded ex vivo.

In some embodiments, the population of cells is expanded by no more than 5, 6, 7, 8, 9, 10, 11, 12, 13, 14, 15, 16, 17, 18, 19, 20, 25, 30, 35, 40, 45, 50, 55, or 60%, for example, as assessed by the number of living cells, compared to the population of cells before it is contacted with the one or more cytokines described above. In some embodiments, the population of cells is expanded by no more than 5%, for example, as assessed by the number of living cells, compared to the population of cells before it is contacted with the one or more cytokines described above. In some embodiments, the population of cells is expanded by no more than 10%, for example, as assessed by the number of living cells, compared to the population of cells before it is contacted with the one or more cytokines described above. In some embodiments, the population of cells is expanded by no more than 15%, for example, as assessed by the number of living cells, compared to the population of cells before it is contacted with the one or more cytokines described above. In some embodiments, the population of cells is expanded by no more than 20%, for example, as assessed by the number of living cells, compared to the population of cells before it is contacted with the one or more cytokines described above. In some embodiments, the population of cells is expanded by no more than 25%, for example, as assessed by the number of living cells, compared to the population of cells before it is contacted with the one or more cytokines described above. In some embodiments, the population of cells is expanded by no more than 30%, for example, as assessed by the number of living cells, compared to the population of cells before it is contacted with the one or more cytokines described above. In some embodiments, the population of cells is expanded by no more than 35%, for example, as assessed by the number of living cells, compared to the population of cells before it is contacted with the one or more cytokines described above. In some embodiments, the population of cells is expanded by no more than 40%, for example, as assessed by the number of living cells, compared to the population of cells before it is contacted with the one or more cytokines described above.

In some embodiments, the population of cells is expanded by no more than 1, 1.5, 2, 2.5, 3, 3.5, 4, 4.5, 5, 6, 7, 8, 9, 10, 11, 12, 16, 20, 24, 36, or 48 hours, for example, as assessed by the number of living cells, compared to the population of cells before it is contacted with the one or more cytokines described above.

In some embodiments, the population of cells is not contacted in vitro with an agent that stimulates a CD3/TCR complex (for example, an anti-CD3 antibody) and/or an agent that stimulates a costimulatory molecule on the surface of the cells (for example, an anti-CD28 antibody), or if contacted, the contacting step is less than 1, 1.5, 2, 2.5, 3, 3.5, 4, 4.5, or 5 hours.

In some embodiments, the population of cells is contacted in vitro with an agent that stimulates a CD3/TCR complex (for example, an anti-CD3 antibody) and/or an agent that stimulates a costimulatory molecule on the surface of the cells (for example, an anti-CD28 antibody) for 20, 21, 22, 23, 24, 25, 26, 27, or 28 hours.

In some embodiments, the population of cells manufactured using the cytokine process provided herein shows a higher percentage of naïve cells among CAR-expressing cells (for example, at least 5, 6, 7, 8, 9, 10, 11, 12, 13, 14, 15, 16, 17, 18, 19, 20, 25, 30, 35, 40, 45, 50, 55, or 60% higher), compared with cells made by an otherwise similar method which further comprises contacting the population of cells with, for example, an agent that binds a CD3/TCR complex (for example, an anti-CD3 antibody) and/or an agent that binds a costimulatory molecule on the surface of the cells (for example, an anti-CD28 antibody).

In some embodiments, the cytokine process provided herein is conducted in cell media comprising no more than 0, 0.5, 1, 1.5, 2, 2.5, 3, 3.5, 4, 4.5, 5, 5.5, 6, 6.5, 7, 7.5, or 8% serum. In some embodiments, the cytokine process provided herein is conducted in cell media comprising a LSD1 inhibitor, a MALT1 inhibitor, or a combination thereof.

### Activation Process

In some embodiments, the present disclosure provides methods of making a population of cells (for example, T cells) that express a chimeric antigen receptor (CAR), e.g., a CAR disclosed herein, e.g., a CCAR disclosed herein. In some embodiments, the population of cells further express a regulatory molecule. In some embodiments, the population of cells express a CCAR disclosed herein. In some embodiments, the population of cells express a CAR disclosed herein and a regulatory molecule disclosed herein. In some embodiments, the method comprises: (i) contacting a population of cells (for example, T cells, for example, T cells isolated from a frozen or fresh leukapheresis product) with an agent that stimulates a CD3/TCR complex and/or an agent that stimulates a costimulatory molecule on the surface of the cells; (ii) contacting the population of cells (for example, T cells) with a nucleic acid molecule (for example, a DNA or RNA molecule) encoding the CAR, e.g., the CCAR, thereby providing a population of cells (for example, T cells) comprising the nucleic acid molecule, and (iii) harvesting the population of cells (for example, T cells) for storage (for example, reformulating the population of cells in cryopreservation media) or administration, wherein: (a) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and step (iii) is performed no later than 26 hours after the beginning of step (i), for example, no later than 22, 23, or 24 hours after the beginning of step (i), for example, no later than 24 hours after the beginning of step (i); (b) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and step (iii) is performed no later than 30 hours after the beginning of step (ii), for example, no later than 22, 23, 24, 25, 26, 27, 28, 29, or 30 hours after the beginning of step (ii); or (c) the population of cells from step (iii) are not expanded, or expanded by no more than 5, 10, 15, 20, 25, 30, 35, or 40%, for example, no more than 10%, for example, as assessed by the number of living cells, compared to the population of cells at the beginning of step (i). In some embodiments, the nucleic acid molecule in step (ii) is a DNA molecule. In some embodiments, the nucleic acid molecule in step (ii) is an RNA molecule. In some embodiments, the nucleic acid molecule in step (ii) is on a viral vector, for example, a viral vector chosen from a lentivirus vector, an adenoviral vector, or a retrovirus vector. In some embodiments, the nucleic acid molecule in step (ii) is on a non-viral vector. In some embodiments, the nucleic acid molecule in step (ii) is on a plasmid. In some embodiments, the nucleic acid molecule in step (ii) is not on any vector. In some embodiments, step (ii) comprises transducing the population of cells (for example, T cells) a viral vector comprising a nucleic acid molecule encoding the CAR, e.g., the CCAR.

In some embodiments, the population of cells (for example, T cells) is collected from an apheresis sample (for example, a leukapheresis sample) from a subject.

In some embodiments, the apheresis sample (for example, a leukapheresis sample) is collected from the subject and shipped as a frozen sample (for example, a cryopreserved sample) to a cell manufacturing facility. Then the frozen apheresis sample is thawed, and T cells (for example, CD4+ T cells and/or CD8+ T cells) are selected from the apheresis sample, for example, using a cell sorting machine (for example, a CliniMACS® Prodigy® device). The selected T cells (for example, CD4+ T cells and/or CD8+ T cells) are then seeded for CART manufacturing using the activation process described herein. In some embodiments, the selected T cells (for example, CD4+ T cells and/or CD8+ T cells) undergo one or more rounds of freeze-thaw before being seeded for CART manufacturing.

In some embodiments, the apheresis sample (for example, a leukapheresis sample) is collected from the subject and shipped as a fresh product (for example, a product that is not frozen) to a cell manufacturing facility. T cells (for example, CD4+ T cells and/or CD8+ T cells) are selected from the apheresis sample, for example, using a cell sorting machine (for example, a CliniMACS® Prodigy® device). The selected T cells (for example, CD4+ T cells and/or CD8+ T cells) are then seeded for CART manufacturing using the activation process described herein. In some embodiments, the selected T cells (for example, CD4+ T cells and/or CD8+ T cells) undergo one or more rounds of freeze-thaw before being seeded for CART manufacturing.

In some embodiments, the apheresis sample (for example, a leukapheresis sample) is collected from the subject. T cells (for example, CD4+ T cells and/or CD8+ T cells) are selected from the apheresis sample, for example, using a cell sorting machine (for example, a CliniMACS® Prodigy® device). The selected T cells (for example, CD4+ T cells and/or CD8+ T cells) are then shipped as a frozen sample (for example, a cryopreserved sample) to a cell manufacturing facility. The selected T cells (for example, CD4+ T cells and/or CD8+ T cells) are later thawed and seeded for CART manufacturing using the activation process described herein.

In some embodiments, cells (for example, T cells) are contacted with anti-CD3 and anti-CD28 antibodies for, for example, 12 hours, followed by transduction with a vector (for example, a lentiviral vector) encoding a CAR, e.g., the CCAR. 24 hours after culture initiation, the cells are washed and formulated for storage or administration.

Without wishing to be bound by theory, brief CD3 and CD28 stimulation may promote efficient transduction of self-renewing T cells. Compared to traditional CART manufacturing approaches, the activation process provided herein does not involve prolonged ex vivo expansion. Similar to the cytokine process, the activation process provided herein also preserves undifferentiated T cells during CART manufacturing.

In some embodiments, the population of cells is contacted with an agent that stimulates a CD3/TCR complex and/or an agent that stimulates a costimulatory molecule on the surface of the cells.

In some embodiments, the agent that stimulates a CD3/TCR complex is an agent that stimulates CD3. In some embodiments, the agent that stimulates a costimulatory molecule is an agent that stimulates CD28, ICOS, CD27, HVEM, LIGHT, CD40, 4-1BB, OX40, DR3, GITR, CD30, TIM1, CD2, CD226, or any combination thereof. In some embodiments, the agent that stimulates a costimulatory molecule is an agent that stimulates CD28. In some embodiments, the agent that stimulates a CD3/TCR complex is chosen from an antibody (for example, a single-domain antibody (for example, a heavy chain variable domain antibody), a peptibody, a Fab fragment, or a scFv), a small molecule, or a ligand (for example, a naturally-existing, recombinant, or chimeric ligand). In some embodiments, the agent that stimulates a CD3/TCR complex is an antibody. In some embodiments, the agent that stimulates a CD3/TCR complex is an anti-CD3 antibody. In some embodiments, the agent that stimulates a costimulatory molecule is chosen from an antibody (for example, a single-domain antibody (for example, a heavy chain variable domain antibody), a peptibody, a Fab fragment, or a scFv), a small molecule, or a ligand (for example, a naturally-existing, recombinant, or chimeric ligand). In some embodiments, the agent that stimulates a costimulatory molecule is an antibody. In some embodiments, the agent that stimulates a costimulatory molecule is an anti-CD28 antibody. In some embodiments, the agent that stimulates a CD3/TCR complex or the agent that stimulates a costimulatory molecule does not comprise a bead. In some embodiments, the agent that stimulates a CD3/TCR complex comprises an anti-CD3 antibody covalently attached to a colloidal polymeric nanomatrix. In some embodiments, the agent that stimulates a costimulatory molecule comprises an anti-CD28 antibody covalently attached to a colloidal polymeric nanomatrix. In some embodiments, the agent that stimulates a CD3/TCR complex and the agent that stimulates a costimulatory molecule comprise T Cell TransAct™.

In some embodiments, the matrix comprises or consists of a polymeric, for example, biodegradable or biocompatible inert material, for example, which is non-toxic to cells. In some embodiments, the matrix is composed of hydrophilic polymer chains, which obtain maximal mobility in aqueous solution due to hydration of the chains. In some embodiments, the mobile matrix may be of collagen, purified proteins, purified peptides, polysaccharides, glycosaminoglycans, or extracellular matrix compositions. A polysaccharide may include for example, cellulose ethers, starch, gum arabic, agarose, dextran, chitosan, hyaluronic acid, pectins, xanthan, guar gum or alginate. Other polymers may include polyesters, polyethers, polyacrylates, polyacrylamides, polyamines, polyethylene imines, polyquaternium polymers, polyphosphazenes, polyvinylalcohols, polyvinylacetates, polyvinylpyrrolidones, block copolymers, or polyurethanes. In some embodiments, the mobile matrix is a polymer of dextran.

In some embodiments, the population of cells is contacted with a nucleic acid molecule encoding a CAR. In some embodiments, the population of cells is transduced with a DNA molecule encoding a CAR. In some embodiments, the population of cells is contacted with a nucleic acid molecule encoding a CCAR. In some embodiments, the population of cells is transduced with a DNA molecule encoding a CCAR. In some embodiments, the population of cells is contacted with a nucleic acid molecule encoding a CAR and a regulatory molecule. In some embodiments, the population of cells is transduced with a DNA molecule encoding a CAR and a regulatory molecule.

In some embodiments, contacting the population of cells with the nucleic acid molecule encoding the CAR, e.g., the CCAR, occurs simultaneously with contacting the population of cells with the agent that stimulates a CD3/TCR complex and/or the agent that stimulates a costimulatory molecule on the surface of the cells described above. In some embodiments, contacting the population of cells with the nucleic acid molecule encoding the CAR, e.g., the CCAR, occurs no later than 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0.5 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule encoding the CAR, e.g., the CCAR, occurs no later than 20 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule encoding the CAR, e.g., the CCAR, occurs no later than 19 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule encoding the CAR, e.g., the CCAR, occurs no later than 18 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule encoding the CAR, e.g., the CCAR, occurs no later than 17 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule encoding the CAR, e.g., the CCAR, occurs no later than 16 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule encoding the CAR, e.g., the CCAR, occurs no later than 15 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule encoding the CAR, e.g., the CCAR, occurs no later than 14 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule encoding the CAR, e.g., the CCAR, occurs no later than 14 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule encoding the CAR, e.g., the CCAR, occurs no later than 13 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule encoding the CAR occurs no later than 12 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule encoding the CAR, e.g., the CCAR, occurs no later than 11 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule encoding the CAR, e.g., the CCAR, occurs no later than 10 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule encoding the CAR, e.g., the CCAR, occurs no later than 9 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule encoding the CAR, e.g., the CCAR, occurs no later than 8 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule encoding the CAR, e.g., the CCAR, occurs no later than 7 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule encoding the CAR, e.g., the CCAR, occurs no later than 6 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule encoding the CAR, e.g., the CCAR, occurs no later than 5 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule encoding the CAR, e.g., the CCAR, occurs no later than 4 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule encoding the CAR, e.g., the CCAR, occurs no later than 3 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule encoding the CAR, e.g., the CCAR, occurs no later than 2 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule encoding the CAR, e.g., the CCAR, occurs no later than 1 hour after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule encoding the CAR, e.g., the CCAR, occurs no later than 30 minutes after the beginning of contacting the population of cells with the agent that stimulates a CD3/TCR complex and/or the agent that stimulates a costimulatory molecule on the surface of the cells described above.

In some embodiments, the population of cells is harvested for storage or administration.

In some embodiments, the population of cells is harvested for storage or administration no later than 72, 60, 48, 36, 32, 31, 30, 29, 28, 27, 26, 25, 24, 23, 22, 21, 20, 19, or 18 hours after the beginning of contacting the population of cells with the agent that stimulates a CD3/TCR complex and/or the agent that stimulates a costimulatory molecule on the surface of the cells described above. In some embodiments, the population of cells is harvested for storage or administration no later than 26 hours after the beginning of contacting the population of cells with the agent that stimulates a CD3/TCR complex and/or the agent that stimulates a costimulatory molecule on the surface of the cells described above. In some embodiments, the population of cells is harvested for storage or administration no later than 25 hours after the beginning of contacting the population of cells with the agent that stimulates a CD3/TCR complex and/or the agent that stimulates a costimulatory molecule on the surface of the cells described above. In some embodiments, the population of cells is harvested for storage or administration no later than 24 hours after the beginning of contacting the population of cells with the agent that stimulates a CD3/TCR complex and/or the agent that stimulates a costimulatory molecule on the surface of the cells described above. In some embodiments, the population of cells is harvested for storage or administration no later than 23 hours after the beginning of contacting the population of cells with the agent that stimulates a CD3/TCR complex and/or the agent that stimulates a costimulatory molecule on the surface of the cells described above. In some embodiments, the population of cells is harvested for storage or administration no later than 22 hours after the beginning of contacting the population of cells with the agent that stimulates a CD3/TCR complex and/or the agent that stimulates a costimulatory molecule on the surface of the cells described above.

In some embodiments, the population of cells is not expanded ex vivo.

In some embodiments, the population of cells is expanded by no more than 5, 6, 7, 8, 9, 10, 11, 12, 13, 14, 15, 16, 17, 18, 19, 20, 25, 30, 35, 40, 45, 50, 55, or 60%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 5%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 10%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 15%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 20%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 25%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 30%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 35%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 40%, for example, as assessed by the number of living cells, compared to the population of cells before it is contacted with the agent that stimulates a CD3/TCR complex and/or the agent that stimulates a costimulatory molecule on the surface of the cells described above.

In some embodiments, the population of cells is expanded by no more than 1, 1.5, 2, 2.5, 3, 3.5, 4, 4.5, 5, 6, 7, 8, 9, 10, 11, 12, 16, 20, 24, 36, or 48 hours, for example, as assessed by the number of living cells, compared to the population of cells before it is contacted with the one or more cytokines described above.

In some embodiments, the activation process is conducted in serum free cell media. In some embodiments, the activation process is conducted in cell media comprising one or more cytokines chosen from: IL-2, IL-15 (for example, hetIL-15 (IL15/sIL-15Ra)), or IL-6 (for example, IL-6/sIL-6Ra). In some embodiments, hetIL-15 comprises the amino acid sequence of NWVNVISDLKKIEDLIQSMHIDATLYTESDVHPSCKVTAMKCFLLELQVISLESGDASIHDTVENL IILANNSLSSNGNVTESGCKECEELEEKNIKEFLQSFVHIVQMFINTSITCPPPMSVEHADIWVKSY SLYSRERYICNSGFKRKAGTSSLTECVLNKATNVAHWTTPSLKCIRDPALVHQRPAPPSTVTTAG VTPQPESLSPSGKEPAASSPSSNNTAATTAAIVPGSQLMPSKSPSTGTTEISSHESSHGTPSQTTAK NWELTASASHQPPGVYPQG (SEQ ID NO: 309). In some embodiments, hetIL-15 comprises an amino acid sequence having at least about 70, 75, 80, 85, 90, 95, or 99% identity to SEQ ID NO: 309. In some embodiments, the activation process is conducted in cell media comprising a LSD1 inhibitor. In some embodiments, the activation process is conducted in cell media comprising a MALT1 inhibitor. In some embodiments, the serum free cell media comprises a serum replacement. In some embodiments, the serum replacement is CTS™ Immune Cell Serum Replacement (ICSR). In some embodiments, the level of ICSR can be, for example, up to 5%, for example, about 1%, 2%, 3%, 4%, or 5%. Without wishing to be bound by theory, using cell media, for example, Rapid Media shown in Table 21 or Table 25, comprising ICSR, for example, 2% ICSR, may improve cell viability during a manufacture process described herein.

In some embodiments, the present disclosure provides methods of making a population of cells (for example, T cells) that express a chimeric antigen receptor (CAR) comprising: (a) providing an apheresis sample (for example, a fresh or cryopreserved leukapheresis sample) collected from a subject; (b) selecting T cells from the apheresis sample (for example, using negative selection, positive selection, or selection without beads); (c) seeding isolated T cells at, for example, 1×10⁶ to 1×10⁷ cells/mL; (d) contacting T cells with an agent that stimulates T cells, for example, an agent that stimulates a CD3/TCR complex and/or an agent that stimulates a costimulatory molecule on the surface of the cells (for example, contacting T cells with anti-CD3 and/or anti-CD28 antibody, for example, contacting T cells with TransAct); (e) contacting T cells with a nucleic acid molecule (for example, a DNA or RNA molecule) encoding the CAR (for example, contacting T cells with a virus comprising a nucleic acid molecule encoding the CAR) for, for example, 6-48 hours, for example, 20-28 hours; and (f) washing and harvesting T cells for storage (for example, reformulating T cells in cryopreservation media) or administration. In some embodiments, step (f) is performed no later than 30 hours after the beginning of step (d) or (e), for example, no later than 22, 23, 24, 25, 26, 27, 28, 29, or 30 hours after the beginning of step (d) or (e).

### Additional Exemplary Manufacturing Methods

In some embodiments, the CAR manufacturing methods described herein (e.g., the Activated Rapid Manufacturing (ARM) process) are compared with a CAR manufacturing process called the “traditional manufacturing (TM)” process. Under the traditional manufacturing process, in some embodiments, cells, e.g., T cells or NK cells are activated, e.g., using anti-CD3/anti-CD28 antibody coated Dynabeads®, contacted with one or more nucleic acid molecules encoding a CAR, and expanded in vitro for, for example, 7, 8, 9, 10, or 11 days, before the cells are harvested. In some embodiments, the cells, e.g., T cells or NK cells, are selected from a fresh or cryopreserved leukapheresis sample, e.g., using positive or negative selection.

### Population of CAR-Expressing Cells Manufactured by the Processes Disclosed Herein

In some embodiments, this disclosure features an immune effector cell (for example, T cell or NK cell), for example, made by any of the manufacturing methods described herein, engineered to express a CAR, e.g., a CCAR, wherein the engineered immune effector cell exhibits an antitumor property. In some embodiments, the immune effector cell is engineered to express a CCAR disclosed herein. In some embodiments, the immune effector cell is engineered to express a CAR disclosed herein and a regulatory molecule disclosed herein.

In some embodiments, the CAR comprises an antigen binding domain, a transmembrane domain, and an intracellular signaling domain. An exemplary antigen is a cancer associated antigen described herein. In some embodiments, the cell (for example, T cell or NK cell) is transformed with the CAR, e.g., the CCAR, and the CAR, e.g., the CCAR, is expressed on the cell surface. In some embodiments, the cell (for example, T cell or NK cell) is transduced with a viral vector encoding the CAR, e.g., the CCAR. In some embodiments, the viral vector is a retroviral vector. In some embodiments, the viral vector is a lentiviral vector. In some such embodiments, the cell may stably express the CAR, e.g., the CCAR. In some embodiments, the cell (for example, T cell or NK cell) is transfected with a nucleic acid, for example, mRNA, cDNA, or DNA, encoding a CAR, e.g., a CCAR. In some such embodiments, the cell may transiently express the CAR, e.g., the CCAR.

In some embodiments, provided herein is a population of cells (for example, immune effector cells, for example, T cells or NK cells) made by any of the manufacturing processes described herein (for example, the cytokine process, or the activation process described herein), engineered to express a CAR.

In some embodiments, the percentage of naïve cells, for example, naïve T cells, for example, CD45RA+ CD45RO− CCR7+ T cells, in the population of cells at the end of the manufacturing process (for example, at the end of the cytokine process or the activation process described herein) (1) is the same as, (2) differs, for example, by no more than 4, 5, 6, 7, 8, 9, 10, 11, 12, 13, 14, or 15%, from, or (3) is increased, for example, by at least 5, 6, 7, 8, 9, 10, 11, 12, 13, 14, 15, 16, 17, 18, 19, 20, 21, 22, 23, 24, or 25%, as compared to, the percentage of naïve cells, for example, naïve T cells, for example, CD45RA+ CD45RO− CCR7+ cells, in the population of cells at the beginning of the manufacturing process (for example, at the beginning of the cytokine process or the activation process described herein). In some embodiments, the population of cells at the end of the manufacturing process (for example, at the end of the cytokine process or the activation process described herein) shows a higher percentage of naïve cells, for example, naïve T cells, for example, CD45RA+ CD45RO− CCR7+ T cells (for example, at least 5, 6, 7, 8, 9, 10, 11, 12, 13, 14, 15, 16, 17, 18, 19, 20, 25, 30, 35, 40, 45, or 50% higher), compared with cells made by an otherwise similar method which lasts, for example, more than 26 hours (for example, which lasts more than 5, 6, 7, 8, 9, 10, 11, or 12 days) or which involves expanding the population of cells in vitro for, for example, more than 3 days (for example, expanding the population of cells in vitro for 3, 4, 5, 6, 7, 8, 9, 10, 11, 12, 13, 14, or 15 days).

In some embodiments, the percentage of naïve cells, for example, naïve T cells, for example, CD45RA+ CD45RO− CCR7+ T cells, in the population of cells at the end of the manufacturing process (for example, at the end of the cytokine process or the activation process described herein) is not less than 20, 25, 30, 35, 40, 45, 50, 55, or 60%.

In some embodiments, the percentage of central memory cells, for example, central memory T cells, for example, CD95+ central memory T cells, in the population of cells at the end of the manufacturing process (for example, at the end of the cytokine process or the activation process described herein) (1) is the same as, (2) differs, for example, by no more than 4, 5, 6, 7, 8, 9, 10, 11, 12, 13, 14, or 15% from, or (3) is decreased, for example, by at least 5, 6, 7, 8, 9, 10, 11, 12, 13, 14, 15, 16, 17, 18, 19, 20, 21, 22, 23, 24, or 25%, as compared to, the percentage of central memory cells, for example, central memory T cells, for example, CD95+ central memory T cells, in the population of cells at the beginning of the manufacturing process (for example, at the beginning of the cytokine process or the activation process described herein). In some embodiments, the population of cells at the end of the manufacturing process (for example, at the end of the cytokine process or the activation process described herein) shows a lower percentage of central memory cells, for example, central memory T cells, for example, CD95+ central memory T cells (for example, at least 5, 6, 7, 8, 9, 10, 11, 12, 13, 14, 15, 16, 17, 18, 19, 20, 25, 30, 35, 40, 45, or 50% lower), compared with cells made by an otherwise similar method which lasts, for example, more than 26 hours (for example, which lasts more than 5, 6, 7, 8, 9, 10, 11, or 12 days) or which involves expanding the population of cells in vitro for, for example, more than 3 days (for example, expanding the population of cells in vitro for 3, 4, 5, 6, 7, 8, 9, 10, 11, 12, 13, 14, or 15 days).

In some embodiments, the percentage of central memory cells, for example, central memory T cells, for example, CD95+ central memory T cells, in the population of cells at the end of the manufacturing process (for example, at the end of the cytokine process or the activation process described herein) is no more than 40, 45, 50, 55, 60, 65, 70, 75, or 80%.

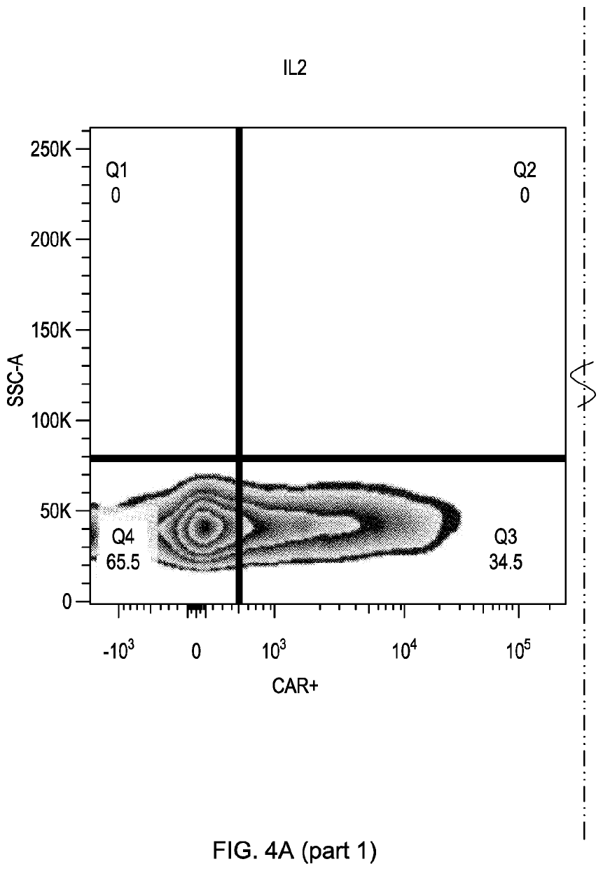

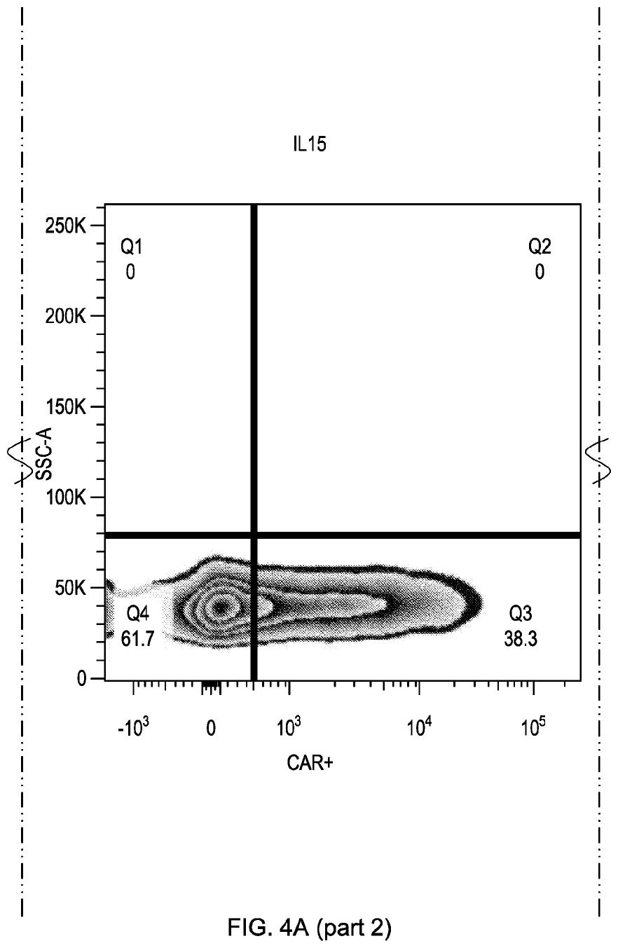

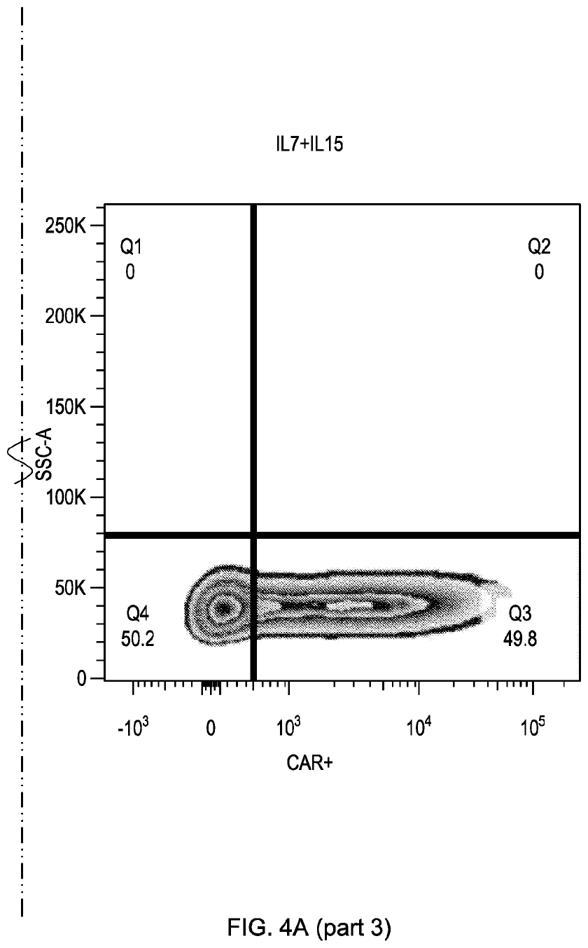

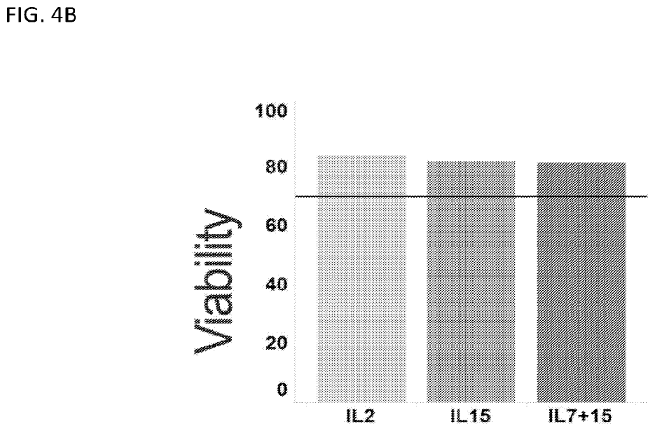

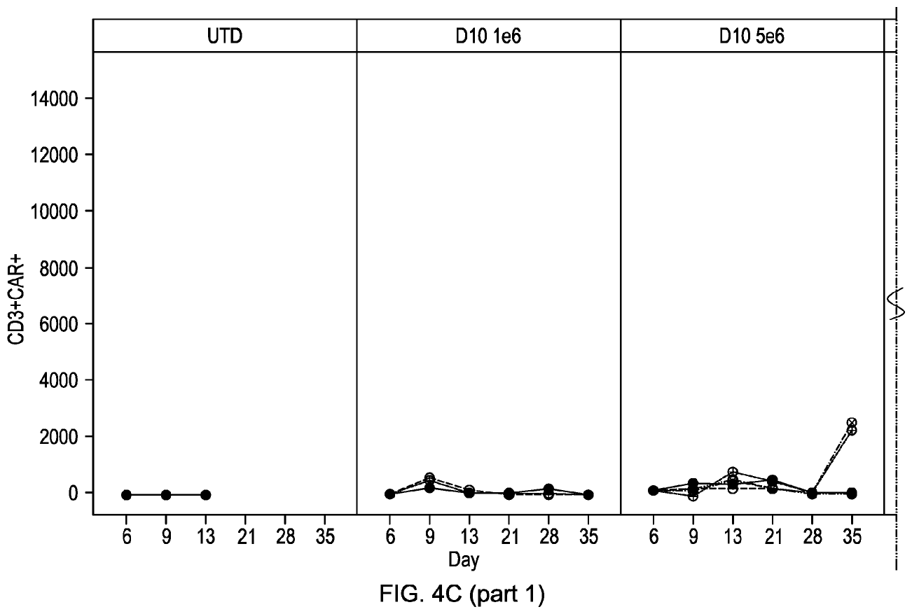

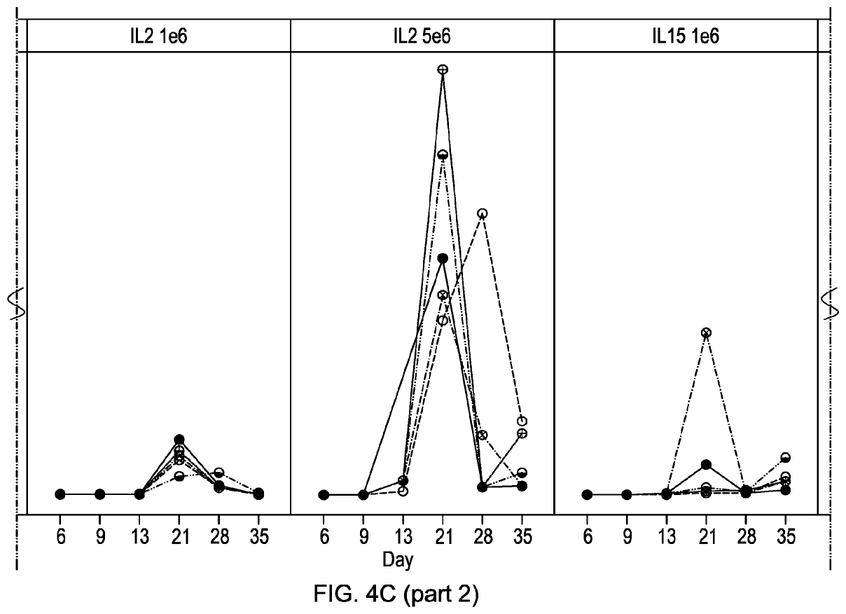

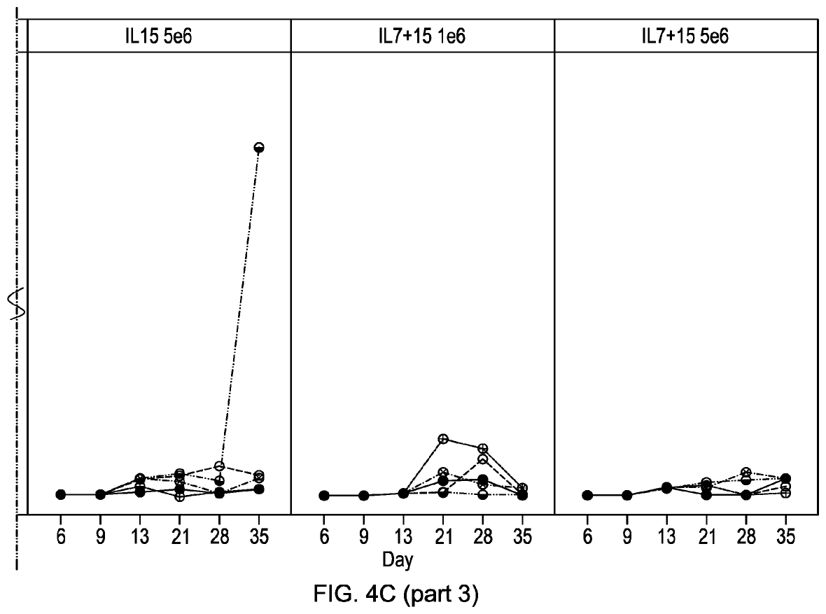

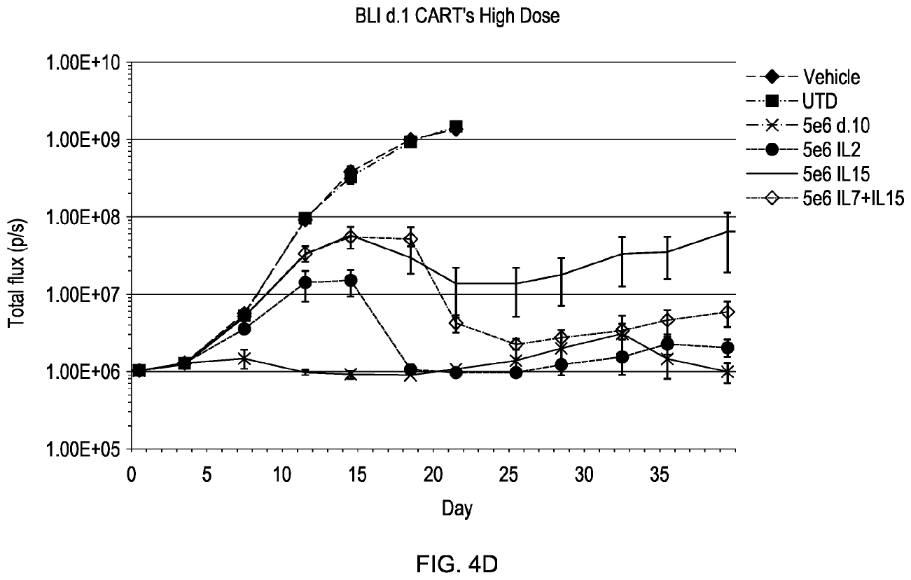

In some embodiments, the population of cells at the end of the manufacturing process (for example, at the end of the cytokine process or the activation process described herein) after being administered in vivo, persists longer or expands at a higher level (for example, at least 20, 25, 30, 35, 40, 45, 50, 55, 60, 65, 70, 75, 80, 85, or 90% higher) (for example, as assessed using methods described in Example 1 with respect to FIG. 4C), compared with cells made by an otherwise similar method which lasts, for example, more than 26 hours (for example, which lasts more than 5, 6, 7, 8, 9, 10, 11, or 12 days) or which involves expanding the population of cells in vitro for, for example, more than 3 days (for example, expanding the population of cells in vitro for 3, 4, 5, 6, 7, 8, 9, 10, 11, 12, 13, 14, or 15 days).

In some embodiments, the population of cells has been enriched for IL6R-expressing cells (for example, cells that are positive for IL6Rα and/or IL6Rβ) prior to the beginning of the manufacturing process (for example, prior to the beginning of the cytokine process or the activation process described 10 herein). In some embodiments, the population of cells comprises, for example, no less than 30, 35, 40, 45, 50, 55, 60, 65, 70, 75, or 80% of IL6R-expressing cells (for example, cells that are positive for IL6Rα and/or IL6Rβ) at the beginning of the manufacturing process (for example, at the beginning of the cytokine process or the activation process described herein).

### Pharmaceutical Composition

Furthermore, the present disclosure provides CAR, e.g., CCAR, —expressing cell compositions and their use in medicaments or methods for treating, among other diseases, cancer or any malignancy or autoimmune diseases involving cells or tissues which express a tumor antigen as described herein. In some embodiments, provided herein are pharmaceutical compositions comprising a CAR, e.g., CCAR, —expressing cell, for example, a plurality of CAR, e.g., CCAR, —expressing cells, made by a manufacturing process described herein (for example, the cytokine process, or the activation process described herein), in combination with one or more pharmaceutically or physiologically acceptable carriers, diluents or excipients. In some embodiments, the CAR-expressing cell expresses a CCAR disclosed herein. In some embodiments, the CAR-expressing cell expresses a CAR disclosed herein and a regulatory molecule disclosed herein.

### Strategies for Regulating Chimeric Antigen Receptors

There are many ways CAR activities can be regulated. In some embodiments, a regulatable CAR (RCAR) where the CAR activity can be controlled is desirable to optimize the safety and efficacy of a CAR therapy. Alternative strategies for regulating the CAR therapy of the instant disclosure include utilizing small molecules or antibodies that degrade a CAR, e.g., a CCAR, or deactivate or turn off CAR activity, e.g., by deleting CAR-expressing cells, e.g., by inducing antibody dependent cell-mediated cytotoxicity (ADCC). For example, CAR-expressing cells described herein may also express an antigen that is recognized by molecules capable of inducing cell death, e.g., ADCC or compliment-induced cell death. For example, CAR expressing cells described herein may also express a receptor capable of being targeted by an antibody or antibody fragment. Examples of such receptors include EpCAM, VEGFR, integrins (e.g., integrins αvβ3, α4, αI¾β3, α4β7, α5β1, aαvβ3, αv), members of the TNF receptor superfamily (e.g., TRAIL-R1 , TRAIL-R2), PDGF Receptor, interferon receptor, folate receptor, GPNMB, ICAM-1 , HLA-DR, CEA, CA-125, MUC1 , TAG-72, IL-6 receptor, 5T4, GD2, GD3, CD2, CD3, CD4, CD5, CD11, CD11a/LFA-1 , CD15, CD18/ITGB2, CD19, CD20, CD22, CD23/IgE Receptor, CD25, CD28, CD30, CD33, CD38, CD40, CD41 , CD44, CD51 , CD52, CD62L, CD74, CD80, CD125, CD147/basigin, CD152/CTLA-4, CD154/CD40L, CD195/CCRS, CD319/SLAMF7, and EGFR, and truncated versions thereof (e.g., versions preserving one or more extracellular epitopes but lacking one or more regions within the cytoplasmic domain). For example, CAR-expressing cells described herein may also express a truncated epidermal growth factor receptor (EGFR) which lacks signaling capacity but retains the epitope that is recognized by molecules capable of inducing ADCC, e.g., cetuximab (ERBITUX®), such that administration of cetuximab induces ADCC and subsequent depletion of the CAR-expressing cells (see, e.g., WO2011/056894, and Jonnalagadda et al., Gene Ther. 2013; 20(8)853-860). Another strategy includes expressing a highly compact marker/suicide gene that combines target epitopes from both CD32 and CD20 antigens in the CAR-expressing cells described herein, which binds rituximab, resulting in selective depletion of the CAR-expressing cells, e.g., by ADCC (see, e.g., Philip et al., Blood. 2014; 124(8)1277-1287). Other methods for depleting CAR-expressing cells described herein include administration of CAMPATH®, a monoclonal anti-CD52 antibody that selectively binds and targets mature lymphocytes, e.g., CAR-expressing cells, for destruction, e.g., by inducing ADCC. In other embodiments, CAR-expressing cells can be selectively targeted using a CAR ligand, e.g., an anti-idiotypic antibody. In some embodiments, the anti-idiotypic antibody can cause effector cell activity, e.g, ADCC or ADC activities, thereby reducing the number of CAR-expressing cells. In other embodiments, the CAR ligand, e.g., the anti-idiotypic antibody, can be coupled to an agent that induces cell killing, e.g., a toxin, thereby reducing the number of CAR-expressing cells. Alternatively, the CAR molecules themselves can be configured such that the activity can be regulated, e.g., turned on and off, as described below.

### Degradation of CCAR Mediated by Degradation Polypeptides and Degradation Compounds

In some embodiments, provided herein is a fusion polypeptide comprising a degradation polypeptide and a heterologous polypeptide. In some embodiments, the degradation polypeptide is fused to the C-terminus or N-terminus of the heterologous polypeptide. In some embodiments, the degradation polypeptide is at the middle of the heterologous polypeptide. In some embodiments, the heterologous polypeptide is a CAR, e.g., a CAR disclosed herein, e.g., a CAR comprising an antigen binding domain, a transmembrane domain, and an intracellular signaling domain. In some embodiments, provided herein is a controllable CAR (CCAR) comprising a degradation polypeptide and a CAR.

In some embodiments, in the presence of a degradation compound disclosed herein, e.g., COF1 or COF2, e.g., an IMiD (e.g., thalidomide and derivatives thereof, e.g., lenalidomide, pomalidomide, and thalidomide) or COF3, e.g., a compound disclosed in Table 29 (e.g., Compound I-112 disclosed in Table 29), the degradation polypeptide alters the level and/or activity of the fusion polypeptide, e.g., CCAR. In some embodiments, in the presence of a degradation compound disclosed herein, the degradation polypeptide increases a post-translational modification and/or degradation of the fusion polypeptide, e.g., CCAR. In some embodiments, post-translational modification can include ubiquitination (e.g., mono- or poly-ubiquitination) of one or more amino acid residues, e.g., one or more of lysine or methionine, in the fusion polypeptide, e.g., CCAR (e.g., one or more of: all or a part of a heterologous polypeptide, e.g., CAR, and/or the degradation polypeptide). In some embodiments, the degradation polypeptide is a degradation polypeptide disclosed in WO2019079569, herein incorporated by reference in its entirety, e.g., a COF1/CRBN-binding polypeptide, COF2/CRBN-binding polypeptide, or COF3/CRBN-binding polypeptide disclosed in WO2019079569, e.g., pages 114-120 of WO2019079569. In some embodiments, the degradation compound is a degradation compound disclosed in WO2019079569, e.g., pages 120-216 of WO2019079569.

In some embodiments, one or more lysine residues of the fusion polypeptide, e.g., CCAR (e.g., all or a part of a heterologous polypeptide, e.g., CAR, and/or the degradation polypeptide) are ubiquitinated. In some embodiments, one or more methionine residues of the fusion polypeptide, e.g., CCAR (e.g., all or a part of a heterologous polypeptide, e.g., CAR, and/or the degradation polypeptide) are ubiquitinated (e.g., mono- or poly-ubiquitinated).

Without wishing to be bound by theory, in some embodiments, inactivation, e.g., degradation, of a fusion polypeptide, e.g., CCAR, described herein can include one, two, three or all of following steps, e.g., in a cell or a reaction mixture:

(1) association of the fusion polypeptide, e.g., CCAR, that comprises the degradation polypeptide to one or more subunits (e.g., CRBN) of a ubiquitin ligase complex (e.g., an E3 ubiquitin ligase complex) in the presence of a degradation compound disclosed herein, e.g., COF1 or COF2, e.g., an IMiD (e.g., thalidomide and derivatives thereof (e.g., lenalidomide)) or COF3, e.g., a compound disclosed in Table 29 (e.g., Compound I-112 disclosed in Table 29);

(2) ubiquitination of the fusion polypeptide, e.g., CCAR (e.g., ubiquitination at a heterologous polypeptide, e.g., CAR, and/or the degradation polypeptide), thereby providing a ubiquitinated fusion polypeptide, e.g., CCAR; and

(3) degradation of the ubiquitinated fusion polypeptide, e.g., CCAR.

In some embodiments, any degradation polypeptide described herein increases a post-translational modification and/or degradation of the fusion polypeptide, e.g., CCAR, in the presence of a degradation compound disclosed herein, e.g., an IMiD or Compound I-112, e.g., relative to the modification and/or degradation in the absence of the degradation compound disclosed herein, e.g., the IMiD or Compound I-112. In one embodiment, the degradation polypeptide increases selective ubiquitination of the fusion polypeptide, e.g., CCAR, in the presence of a degradation compound disclosed herein, e.g., an IMiD or Compound I-112, e.g., relative to the ubiquitination in the absence of the degradation compound disclosed herein, e.g., the IMiD or Compound I-112.

In some embodiments, provided herein is a nucleic acid molecule encoding a fusion polypeptide, e.g., CCAR, disclosed herein. In some embodiments, provided herein is a vector comprising the nucleic acid molecule. In some embodiments, provided herein is a cell comprising the nucleic acid molecule or the vector.

In some embodiments, provided herein is a method of selectively regulating (e.g., degrading) a fusion polypeptide, e.g., CCAR (e.g., a fusion polypeptide, e.g., CCAR, comprising a degradation polypeptide and a heterologous polypeptide, e.g., CAR). Such methods can include contacting a cell comprising any of the fusion polypeptides, e.g., CCARs, described herein or a nucleic acid encoding such a fusion polypeptide, e.g., CCAR, with any of the degradation compounds described herein. In some embodiments, the cell is contacted with the degradation compound in vivo. In some embodiments, the cell is contacted with the degradation compound in ex vivo. As used herein, “selectively degrading” a fusion polypeptide, e.g., CCAR, or target polypeptide, or the like, refers to an increase in degradation (e.g. an increased level and/or rate of degradation, e.g., at least 5%, 10%, 15%, 20%, 25%, 30%, 35%, 40%, 45%, 50%, 55%, 60%, 65%, 70%, 75%, 80%, 85%, 90%, 95%, 100%, 200%, 500%, 10 times, 100 times, 1,000 times, or higher) of the fusion polypeptide, e.g., CCAR, or target polypeptide, relative to a reference polypeptide, e.g., a polypeptide without a degradation polypeptide.

In some embodiments, the present disclosure provides methods comprising administering a fusion polypeptide, e.g., CCAR, of the present disclosure as a therapy. In some embodiments, such administration is in the form of cells (e.g., autologous or allogeneic host cells) expressing the fusion polypeptide, e.g., CCAR, of the present disclosure to the subject. Accordingly, through administration of a degradation compound (either in vivo or ex vivo), the expression of the therapeutic (e.g., the heterologous polypeptide, e.g., CAR) can be regulated. Accordingly, through administration of a degradation compound (either in vivo or ex vivo), the expression of the therapeutic (e.g., the heterologous polypeptide, e.g., CAR) can be regulated. Thus, expression of known synthetic therapeutic proteins or transmembrane receptors (e.g., a fusion polypeptide, e.g., CCAR, e.g., as described herein, e.g., comprising a domain that includes a CAR molecule described herein) can be regulated. In one embodiment, the subject has a disorder described herein, e.g., the subject has cancer, e.g., the subject has a cancer which expresses a target antigen described herein. In one embodiment, the subject is a human.

### Degradation Polypeptides

In some embodiments, a degradation polypeptide is derived from an amino acid sequence and/or structural motif (e.g., a domain) that binds to one or more components of a ubiquitin ligase complex (e.g., the E3 ubiquitin ligase complex) in the presence of a degradation compound disclosed herein, e.g., COF1, or COF2, an IMiD, e.g., a thalidomide class of compounds (e.g., lenalidomide, pomalidomide, and thalidomide) or COF3, e.g., a compound disclosed in Table 29, e.g., Compound I-112 disclosed in Table 29. In some embodiments, the degradation polypeptide comprises a zinc finger domain (e.g., a zinc finger 2 domain) or a portion thereof. In some embodiments, the degradation polypeptide comprises a β turn. In some embodiments, the degradation polypeptide comprises an IKZF polypeptide or a structural motif thereof. In some embodiments, the IKZF polypeptide is an IKZF1 polypeptide, an IKZF2 polypeptide, an IKZF3 polypeptide, an IKZF2 polypeptide having H141Q substitution (numbered according to SEQ ID NO: 330), or an IKZF4 polypeptide having H188Q substitution (numbered according to SEQ ID NO: 331).

In some embodiments, the degradation polypeptide comprises a β turn of an Ikaros family of transcription factors, e.g., IKZF1 or IKZF3, or a sequence substantially identical thereto (e.g., at least 85, 87, 90, 95, 97, 98, 99, or 100% identical thereto). In some embodiments, the degradation polypeptide comprises a β hairpin of IKZF1 or IKZF3, or a sequence substantially identical thereto (e.g., at least 85, 87, 90, 95, 97, 98, 99, or 100% thereto). In some embodiments, the degradation polypeptide comprises a beta strand of IKZF1 or IKZF3, or a sequence substantially identical thereto (e.g., at least 85, 87, 90, 95, 97, 98, 99, or 100% identical thereto). In some embodiments, the degradation polypeptide comprises an alpha helix of IKZF1 or IKZF3, or a sequence substantially identical thereto (e.g., at least 85, 87, 90, 95, 97, 98, 99, or 100% identical thereto). In some embodiments, the degradation polypeptide comprises, from N-terminus to C-terminus, a first beta strand, a beta hairpin, a second beta strand, and a first alpha helix of IKZF1 or IKZF3. In some embodiments, the degradation polypeptide comprises, from N-terminus to C-terminus, a first beta strand, a beta hairpin, a second beta strand, a first alpha helix, and a second alpha helix of IKZF1 or IKZF3. In some embodiments, the beta hairpin and the second alpha helix are separated by no more than 60, 50, 40, or 30 amino acid residues.

In some embodiments, the degradation polypeptide comprises about 10 to about 95 amino acid residues, about 15 to about 90 amino acid residues, about 20 to about 85 amino acid residues, about 25 to about 80 amino acid residues, about 30 to about 75 amino acid residues, about 35 to about 70 amino acid residues, about 40 to about 65 amino acid residues, about 45 to about 65 amino acid residues, about 50 to about 65 amino acid residues, or about 55 to about 65 amino acid residues of IKZF1 (e.g., SEQ ID NO: 329) or IKZF3 (e.g., SEQ ID NO: 328) or a sequence substantially identical thereto (e.g., at least 85, 87, 90, 95, 97, 98, 99, or 100% identical thereto). In some embodiments, the degradation polypeptide comprises at least 10 amino acids, at least 15 amino acids, at least 20 amino acids, at least 25 amino acids, at least 30 amino acids, at least 35 amino acids, at least 40 amino acids, at least 45 amino acids, at least 50 amino acids, at least 55 amino acids, at least 60 amino acids, at least 65 amino acids, at least 70 amino acids, at least 75 amino acids, at least 80 amino acids, at least 85 amino acids, at least 90 amino acids, at least 90 amino acids, or at least 95 amino acids of IKZF1 (e.g., SEQ ID NO: 329) or IKZF3 (e.g., SEQ ID NO: 328), or a sequence substantially identical thereto (e.g., at least 85, 87, 90, 95, 97, 98, 99, or 100% identical thereto). In some embodiments, the degradation polypeptide comprises or consists of an amino acid sequence selected from the group consisting of SEQ ID NOs: 310-315, 320-324, 337-339, 360-361, 367-369 and 374 (or a sequence having at least 85, 87, 90, 95, 97, 98, 99, or 100% identity thereto). In some embodiments, the degradation polypeptide comprises or consists of the amino acid sequence of SEQ ID NO: 312. In some embodiments, the degradation compound is a thalidomide class of compounds (e.g., lenalidomide, pomalidomide, and thalidomide), e.g., as described herein. In some embodiments, the degradation compound is COF1 or COF2.

In some embodiments, the degradation polypeptide comprises a β turn of IKZF2, or a sequence substantially identical thereto (e.g., at least 85, 87, 90, 95, 97, 98, 99, or 100% identical thereto). In some embodiments, the degradation polypeptide comprises a β hairpin of IKZF2, or a sequence substantially identical thereto (e.g., at least 85, 87, 90, 95, 97, 98, 99, or 100% identical thereto). In some embodiments, the degradation polypeptide comprises a beta strand of IKZF2, or a sequence substantially identical thereto (e.g., at least 85, 87, 90, 95, 97, 98, 99, or 100% identical thereto). In some embodiments, the degradation polypeptide comprises an alpha helix of IKZF2, or a sequence substantially identical thereto (e.g., at least 85, 87, 90, 95, 97, 98, 99, or 100% identical thereto). In some embodiments, the degradation polypeptide comprises, from N-terminus to C-terminus, a first beta strand, a beta hairpin, a second beta strand, and a first alpha helix of IKZF2. In some embodiments, the degradation polypeptide comprises, from N-terminus to C-terminus, a first beta strand, a beta hairpin, a second beta strand, a first alpha helix, and a second alpha helix of IKZF2. In some embodiments, the beta hairpin and the second alpha helix are separated by no more than 60, 50, 40, or 30 amino acid residues.

In some embodiments, the degradation polypeptide comprises about 10 to about 95 amino acid residues, about 15 to about 90 amino acid residues, about 20 to about 85 amino acid residues, about 25 to about 80 amino acid residues, about 30 to about 75 amino acid residues, about 35 to about 70 amino acid residues, about 40 to about 65 amino acid residues, about 45 to about 65 amino acid residues, about 50 to about 65 amino acid residues, or about 55 to about 65 amino acid residues of IKZF2 (e.g., SEQ ID NO: 21) or a sequence substantially identical thereto (e.g., at least 85, 87, 90, 95, 97, 98, 99, or 100% identical thereto). In some embodiments, the degradation polypeptide comprises at least 10 amino acids, at least 15 amino acids, at least 20 amino acids, at least 25 amino acids, at least 30 amino acids, at least 35 amino acids, at least 40 amino acids, at least 45 amino acids, at least 50 amino acids, at least 55 amino acids, at least 60 amino acids, at least 65 amino acids, at least 70 amino acids, at least 75 amino acids, at least 80 amino acids, at least 85 amino acids, at least 90 amino acids, at least 90 amino acids, or at least 95 amino acids of IKZF2 (e.g., SEQ ID NO: 21), or a sequence substantially identical thereto (e.g., at least 85, 87, 90, 95, 97, 98, 99, or 100% identical thereto). In some embodiments, the degradation polypeptide comprises or consists of an amino acid sequence selected from the group consisting of SEQ ID NOs: 375-377 (or a sequence having at least 85, 87, 90, 95, 97, 98, 99, or 100% identity thereto). In some embodiments, the degradation polypeptide comprises or consists of the amino acid sequence of SEQ ID NO: 375. In some embodiments, the degradation compound is a compound disclosed in Table 29, e.g., Compound I-112 disclosed in Table 29. In some embodiments, the degradation compound is COF3.

In some embodiments, exemplary degradation polypeptides are disclosed in Table 30. Table 31 discloses exemplary full-length sequences of IKZF1, IKZF2, IKZF3, IKZF4, and IKZF5 or fragment thereof.

TABLE 30
Exemplary degradation polypeptides

| SEQ ID | | |
|---|---|---|
| NO | Comment | Sequence |
| SEQ ID | IKZF3 | MHKRSHTGERPFQCNQCGASFTQKGNLLRHI |
| NO: 310 | 136-180 | KLHTGEKPFKCHLCNTASAEARHIKAEMG |
| | and | |
| | 236-249 | |
| | (with | |
| | N- | |
| | terminal | |
| | meth- | |
| | ionine) | |
| SEQ ID | IKZF3 | HKRSHTGERPFQCNQCGASFTQKGNLLRHIK |
| NO: 312 | 136-180 | LHTGEKPFKCHLCNTASAEARHIKAEMG |
| | and | |
| | 236-249 | |
| | (without | |
| | N- | |
| | terminal | |
| | meth- | |
| | ionine) | |
| SEQ ID | Lysine- | MHRRSHTGERPFQCNQCGASFTQRGNLLRHI |
| NO: 311 | free | RLHTGERPFRCHLCNTASAEARHIRAEMG |
| | IKZF3 | |
| | 136-180 | |
| | and | |
| | 236-249 | |
| | variant | |
| | (with | |
| | N- | |
| | terminal | |
| | meth- | |
| | ionine) | |
| SEQ ID | Lysine- | HRRSHTGERPFQCNQCGASFTQRGNLLRHIR |
| NO: 313 | free | LHTGERPFRCHLCNTASAEARHIRAEMG |
| | IKZF3 | |
| | 136-180 | |
| | and | |
| | 236-249 | |
| | variant | |
| | (without | |
| | N- | |
| | terminal | |
| | meth- | |
| | ionine) | |
| SEQ ID | IKZF3 | MHKRSHTGERPFQCNQCGASFTQKGNLLRHI |
| NO: 360 | 136-180 | KLHTGEKPFKCHLCN |
| | (with | |
| | N- | |
| | terminal | |
| | meth- | |
| | ionine) | |
| SEQ ID | IKZF3 | HKRSHTGERPFQCNQCGASFTQKGNLLRHIK |
| NO: 314 | 136-180 | LHTGEKPFKCHLCN |
| | (without | |
| | N- | |
| | terminal | |
| | meth- | |
| | ionine) | |
| SEQ ID | Lysine- | HRRSHTGERPFQCNQCGASFTQRGNLLRHIR |
| NO: 337 | free | LHTGERPFRCHLCN |
| | IKZF3 | |
| | 136-180 | |
| SEQ ID | IKZF3 | MHKRSHTGERPFQCNQCGASFTQKGNLLRHI |
| NO: 361 | 136-170 | KLHTG |
| | (with | |
| | N- | |
| | terminal | |
| | meth- | |
| | ionine) | |
| SEQ ID | IKZF3 | HKRSHTGERPFQCNQCGASFTQKGNLLRHIK |
| NO: 315 | 136-170 | LHTG |
| | (without | |
| | N- | |
| | terminal | |
| | meth- | |
| | ionine) | |
| SEQ ID | Lysine- | HRRSHTGERPFQCNQCGASFTQRGNLLRHIR |
| NO: 338 | free | LHTG |
| | IKZF3 | |
| | 136-170 | |
| SEQ ID | IKZF3 | MHTGERPFQCNQCGASFTQKGNLLRHIKLHT |
| NO: 362 | 140-170 | G |
| | (with | |
| | N- | |
| | terminal | |
| | meth- | |
| | ionine) | |
| SEQ ID | IKZF3 | HTGERPFQCNQCGASFTQKGNLLRHIKLHTG |
| NO: 316 | 140-170 | |
| | (without | |
| | N- | |
| | terminal | |
| | meth- | |
| | ionine) | |
| SEQ ID | IKZF3 | MHTGERPFQCNQCGASFTQKGNLLRHIKLHT |
| NO: 363 | 140-169 | |
| | (with | |
| | N- | |
| | terminal | |
| | meth- | |
| | ionine) | |
| SEQ ID | IKZF3 | HTGERPFQCNQCGASFTQKGNLLRHIKLHT |
| NO: 333 | 140-169 | |
| | (without | |
| | N- | |
| | terminal | |
| | meth- | |
| | ionine) | |
| SEQ ID | IKZF3 | MTGERPFQCNQCGASFTQKGNLLR |
| NO: 364 | 141-163 | |
| | (with | |
| | N- | |
| | terminal | |
| | meth- | |
| | ionine) | |
| SEQ ID | IKZF3 | TGERPFQCNQCGASFTQKGNLLR |
| NO: 317 | 141-163 | |
| | (without | |
| | N- | |
| | terminal | |
| | meth- | |
| | ionine) | |
| SEQ ID | IKZF3 | MPFQCNQCGASFTQKGNLLRHIKLHTG |
| NO: 365 | 145-170 | |
| | (with | |
| | N- | |
| | terminal | |
| | meth- | |
| | ionine) | |
| SEQ ID | IKZF3 | PFQCNQCGASFTQKGNLLRHIKLHTG |
| NO: 318 | 145-170 | |
| | (without | |
| | N- | |
| | terminal | |
| | meth- | |
| | ionine) | |
| SEQ ID | IKZF3 | MPFQCNQCGASF |
| NO: 366 | 145-155 | |
| | (with | |
| | N- | |
| | terminal | |
| | meth- | |
| | ionine) | |
| SEQ ID | IKZF3 | PFQCNQCGASF |
| NO: 319 | 145-155 | |
| | (without | |
| | N- | |
| | terminal | |
| | meth- | |
| | ionine) | |
| SEQ ID | IKZF3 | TASAEARHIKAEMG |
| NO: 320 | 236-249 | |
| SEQ ID | Lysine- | TASAEARHIRAEMG |
| NO: 339 | free | |
| | IKZF3 | |
| | 236-249 | |
| SEQ ID | IKZF3 | MHKRSHTGERPFQCNQCGASFTQKGNLLRH |
| NO: 321 | 136-180 | IKLHTGEKPFKCHLCNTASAEARHIRAEMG |
| | and | |
| | 236-249 | |
| | K245R | |
| | (with | |
| | N- | |
| | terminal | |
| | meth- | |
| | ionine) | |
| SEQ ID | IKZF3 | HKRSHTGERPFQCNQCGASFTQKGNLLRHIK |
| NO: 367 | 136-180 | LHTGEKPFKCHLCNTASAEARHIRAEMG |
| | and | |
| | 236-249 | |
| | K245R | |
| | (without | |
| | N- | |
| | terminal | |
| | meth- | |
| | ionine) | |
| SEQ ID | IKZF3 | MHKRSHTGERPFQCNQCGASFTQKGNLLRHI |
| NO: 322 | 136-180 | KLHTGEKPFKCHLCNTASAEARHISAEMG |
| | and | |
| | 236-249 | |
| | K245S | |
| | (with | |
| | N- | |
| | terminal | |
| | meth- | |
| | ionine) | |
| SEQ ID | IKZF3 | HKRSHTGERPFQCNQCGASFTQKGNLLRHIK |
| NO: 374 | 136-180 | LHTGEKPFKCHLCNTASAEARHISAEMG |
| | and | |
| | 236-249 | |
| | K245S | |
| | (without | |
| | N- | |
| | terminal | |
| | meth- | |
| | ionine) | |
| SEQ ID | IKZF3 | MHKRSHTGERPFQCNQCGASFTQKGNLLRHI |
| NO: 323 | 136-180 | KLHTGEKPFKCHLCNMALEKMALEKMALE |
| | MALEK | |
| | (with | |
| | N- | |
| | terminal | |
| | meth- | |
| | ionine) | |
| SEQ ID | IKZF3 | HKRSHTGERPFQCNQCGASFTQKGNLLRHIK |
| NO: 368 | 136-180 | LHTGEKPFKCHLCNMALEKMALEKMALE |
| | MALEK | |
| | (without | |
| | N- | |
| | terminal | |
| | meth- | |
| | ionine) | |
| SEQ ID | IKZF3 | MHKRSHTGERPFQCNQCGASFTQKGNLLRHI |
| NO: 324 | 136-170 | KLHTGMALEKMALEKMALE |
| | MALEK | |
| | (with | |
| | N- | |
| | terminal | |
| | meth- | |
| | ionine) | |
| SEQ ID | IKZF3 | HKRSHTGERPFQCNQCGASFTQKGNLLRHIK |
| NO: 369 | 136-170 | LHTGMALEKMALEKMALE |
| | MALEK | |
| | (without | |
| | N- | |
| | terminal | |
| | meth- | |
| | ionine) | |
| SEQ ID | IKZF3 | MHTGERPFQCNQCGASFTQKGNLLRHIKLHT |
| NO: 325 | 140-170 | GMALEKMALEKMALE |
| | MALEK | |
| | (with | |
| | N- | |
| | terminal | |
| | meth- | |
| | ionine) | |
| SEQ ID | IKZF3 | HTGERPFQCNQCGASFTQKGNLLRHIKLHTG |
| NO: 370 | 140-170 | MALEKMALEKMALE |
| | MALEK | |
| | (without | |
| | N- | |
| | terminal | |
| | meth- | |
| | ionine) | |
| SEQ ID | IKZF3 | MTGERPFQCNQCGASFTQKGNLLRMALEKMA |
| NO: 326 | 141-163 | LEKMALE |
| | MALEK | |
| | (with | |
| | N- | |
| | terminal | |
| | meth- | |
| | ionine) | |
| SEQ ID | IKZF3 | TGERPFQCNQCGASFTQKGNLLRMALEKMAL |
| NO: 371 | 141-163 | EKMALE |
| | MALEK | |
| | (without | |
| | N- | |
| | terminal | |
| | meth- | |
| | ionine) | |
| SEQ ID | IKZF3 | MPFQCNQCGASFMALEKMALEKMALE |
| NO: 327 | 145-155 | |
| | MALEK | |
| | (with | |
| | N- | |
| | terminal | |
| | meth- | |
| | ionine) | |
| SEQ ID | IKZF3 | PFQCNQCGASFMALEKMALEKMALE |
| NO: 372 | 145-155 | |
| | MALEK | |
| | (without | |
| | N- | |
| | terminal | |
| | meth- | |
| | ionine) | |
| SEQ ID | IKZF3 | MHKRSHTGERPFHCNQCGASFTQKGNLLRHI |
| NO: 334 | 136-180 | KLHTGEKPFKCHLCN |
| | Q147H | |
| | (with | |
| | N- | |
| | terminal | |
| | meth- | |
| | ionine) | |
| SEQ ID | IKZF3 | HKRSHTGERPFHCNQCGASFTQKGNLLRHIK |
| NO: 373 | 136-180 | GLHTEKPFKCHLCN |
| | Q147H | |
| | (without | |
| | N- | |
| | terminal | |
| | meth- | |
| | ionine) | |
| SEQ ID | IKZF2 | HKRSHTGERPFHCNQCGASFTQKGNLLRHIK |
| NO: 375 | 130-174 | LHSGEKPFKCPFCSAGQVMSHHVPPMED |
| | and | |
| | 230-243 | |
| SEQ ID | IKZF2 | HKRSHTGERPFHCNQCGASFTQKGNLLRHIK |
| NO: 376 | 130-174 | LHSGEKPFKCPFCS |
| SEQ ID | IKZF2 | AGQVMSHHVPPMED |
| NO: 377 | 230-243 | |

TABLE 31
Exemplary IKZF sequences

| SEQ | | |
|---|---|---|
| ID | Com- | |
| NO | ment | Sequence |
| SEQ | IKZF1 | MDADEGQDMSQVSGKESPPVSDTPDEGDEPMPIPEDL |
| ID | full | STTSGGQQSSKSDRVVASNVKVETQSDEENGRACEMN |
| NO: | length | GEECAEDLRMLDASGEKMNGSHRDQGSSALSGVGGIR |
| 329 | | LPNGKLKCDICGIICIGPNVLMVHKRSHTGERPFQCN |
| | | QCGASFTQKGNLLRHIKLHSGEKPFKCHLCNYACRRR |
| | | DALTGHLRTHSVGKPHKCGYCGRSYKQRSSLEEHKER |
| | | CHNYLESMGLPGTLYPVIKEETNHSEMAEDLCKIGSE |
| | | RSLVLDRLASNVAKRKSSMPQKFLGDKGLSDTPYDSS |
| | | ASYEKENEMMKSHVMDQAINNAINYLGAESLRPLVQT |
| | | PPGGSEVVPVISPMYQLHKPLAEGTPRSNHSAQDSAV |
| | | ENLLLLSKAKLVPSEREASPSNSCQDSTDTESNNEEQ |
| | | RSGLIYLTNHIAPHARNGLSLKEEHRAYDLLRAASEN |
| | | SQDALRVVSTSGEQMKVYKCEHCRVLFLDHVMYTIHM |
| | | GCHGFRDPFECNMCGYHSQDRYEFSSHITRGEHRFHM |
| | | S |
| SEQ | IKZF2 | METEAIDGYITCDNELSPEREHSNMAIDLTSSTPNGQ |
| ID | full | HASPSHMTSTNSVKLEMQSDEECDRKPLSREDEIRGH |
| NO: | length | DEGSSLEEPLIESSEVADNRKVQELQGEGGIRLPNGK |
| 330 | | LKCDVCGMVCIGPNVLMVHKRSHTGERPFHCNQCGAS |
| | | FTQKGNLLRHIKLHSGEKPFKCPFCSYACRRRDALTG |
| | | HLRTHSVGKPHKCNYCGRSYKQRSSLEEHKERCHNYL |
| | | QNVSMEAAGQVMSHHVPPMEDCKEQEPIMDNNISLVP |
| | | FERPAVIEKLTGNMGKRKSSTPQKFVGEKLMRFSYPD |
| | | IHFDMNLTYEKEAELMQSHMMDQAINNAITYLGAEAL |
| | | HPLMQHPPSTIAEVAPVISSAYSQVYHPNRIERPISR |
| | | ETADSHENNMDGPISLIRPKSRPQEREASPSNSCLDS |
| | | TDSESSHDDHQSYQGHPALNPKRKQSPAYMKEDVKAL |
| | | DTTKAPKGSLKDIYKVFNGEGEQIRAFKCEHCRVLFL |
| | | DHVMYTIHMGCHGYRDPLECNICGYRSQDRYEFSSHI |
| | | VRGEHTFH |
| SEQ | IKZF3 | MEDIQTNAELKSTQEQSVPAESAAVLNDYSLTKSHEM |
| ID | full | ENVDSGEGPANEDEDIGDDSMKVKDEYSERDENVLKS |
| NO: | length | EPMGNAEEPEIPYSYSREYNEYENIKLERHVVSFDSS |
| 328 | | RPTSGKMNCDVCGLSCISFNVLMVHKRSHTGERPFQC |
| | | NQCGASFTQKGNLLRHIKLHTGEKPFKCHLCNYACQR |
| | | RDALTGHLRTHSVEKPYKCEFCGRSYKQRSSLEEHKE |
| | | RCRTFLQSTDPGDTASAEARHIKAEMGSERALVLDRL |
| | | ASNVAKRKSSMPQKFIGEKRHCFDVNYNSSYMYEKES |
| | | ELIQTRMMDQAINNAISYLGAEALRPLVQTPPAPTSE |
| | | MVPVISSMYPIALTRAEMSNGAPQELEKKSIHLPEKS |
| | | VPSERGLSPNNSGHDSTDTDSNHEERQNHIYQQNHMV |
| | | LSRARNGMPLLKEVPRSYELLKPPPICPRDSVKVINK |
| | | EGEVMDVYRCDHCRVLFLDYVMFTIHMGCHGFRDPFE |
| | | CNMCGYRSHDRYEFSSHIARGEHRALLK |
| SEQ | IKZF4 | MHTPPALPRRFQGGGRVRTPGSHRQGKDNLERDPSGG |
| ID | full | CVPDFLPQAQDSNHFIMESLFCESSGDSSLEKEFLGA |
| NO: | length | PVGPSVSTPNSQHSSPSRSLSANSIKVEMYSDEESSR |
| 331 | | LLGPDERLLEKDDSVIVEDSLSEPLGYCDGSGPEPHS |
| | | PGGIRLPNGKLKCDVCGMVCIGPNVLMVHKRSHTGER |
| | | PFHCNQCGASFTQKGNLLRHIKLHSGEKPFKCPFCNY |
| | | ACRRRDALTGHLRTHSVSSPTVGKPYKCNYCGRSYKQ |
| | | QSTLEEHKERCHNYLQSLSTEAQALAGQPGDEIRDLE |
| | | MVPDSMLHSSSERPTFIDRLANSLTKRKRSTPQKFVG |
| | | EKQMRFSLSDLPYDVNSGGYEKDVELVAHHSLEPGFG |
| | | SSLAFVGAEHLRPLRLPPTNCISELTPVISSVYTQMQ |
| | | PLPGRLELPGSREAGEGPEDLADGGPLLYRPRGPLTD |
| | | PGASPSNGCQDSTDTESNHEDRVAGVVSLPQGPPPQP |
| | | PPTIVVGRHSPAYAKEDPKPQEGLLRGTPGPSKEVLR |
| | | VVGESGEPVKAFKCEHCRILFLDHVMFTIHMGCHGFR |
| | | DPFECNICGYHSQDRYEFSSHIVRGEHKVG |
| SEQ | IKZF5 | MGEKKPEPLDFVKDFQEYLTQQTHHVNMISGSVSGDK |
| ID | full | EAEALQGAGTDGDQNGLDHPSVEVSLDENSGMLVDGF |
| NO: | length | ERTFDGKLKCRYCNYASKGTARLIEHIRIHTGEKPHR |
| 332 | | CHLCPFASAYERHLEAHMRSHTGEKPYKCELCSFRCS |
| | | DRSNLSHHRRRKHKMVPIKGTRSSLSSKKMWGVLQKK |
| | | TSNLGYSRRALINLSPPSMVVQKPDYLNDFTHEIPNI |
| | | QTDSYESMAKTTPTGGLPRDPQELMVDNPLNQLSTLA |
| | | GQLSSLPPENQNPASPDVVPCPDEKPFMIQQPSTQAV |
| | | VSAVSASIPQSSSPTSPEPRPSHSQRNYSPVAGPSSE |
| | | PSAHTSTPSIGNSQPSTPAPALPVQDPQLLHHCQHCD |
| | | MYFADNILYTIHMGCHGYENPFQCNICGCKCKNKYDF |
| | | ACHFARGQHNQH |

### Degradation Compounds

Disclosed herein are degradation compounds that can, e.g., increase the ubiquitination and/or degradation of a fusion polypeptide, e.g., CCAR, comprising a degradation polypeptide.

In some embodiments, the degradation compound is an immunomodulatory imide drug (IMiD). In some embodiments, the degradation compound comprises a member of the thalidomide class of compounds. In some embodiments, members of the thalidomide class of compounds include, but are not limited to, lenalidomide (CC-5013), pomalidomide (CC-4047 or ACTIMID), thalidomide, or salts or derivatives thereof. In some embodiments, the degradation compound can be a mixture of one, two, three, or more members of the thalidomide class of compounds. Thalidomide analogs and immunomodulatory properties of thalidomide analogs are described in Bodera and Stankiewicz, Recent Pat Endocr Metab Immune Drug Discov. 2011 September; 5(3):192-6, which is hereby incorporated by reference in its entirety. The structural complex of thalidomide analogs and the E3 ubiquitin is described in Gandhi et al., Br J Haematol. 2014 March; 164(6):811-21, which is hereby incorporated by reference in its entirety. The modulation of the E3 ubiquitin ligase by thalidomide analogs is described in Fischer et al., Nature. 2014 Aug. 7; 512(7512):49-53, which is hereby incorporated by reference in its entirety.

In some embodiments, the degradation compound is a compound of Formula (I) (COF1), wherein the COF1 is:

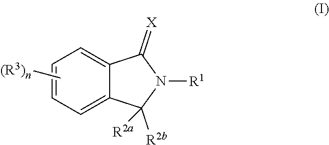

or a pharmaceutically acceptable salt, ester, hydrate, solvate, or tautomer thereof, wherein:

X is O or S;

R¹ is C₁-C₆ alkyl, C₂-C₆ alkenyl, C₂-C₆ alkynyl, C₁-C₆ heteroalkyl, carbocyclyl, heterocyclyl, aryl, or heteroaryl, each of which is independently and optionally substituted by one or more R⁴;

each of R^{2a} and R^{2b} is independently hydrogen or C₁-C₆ alkyl; or R^{2a} and R^{2b} together with the carbon atom to which they are attached form a carbonyl group or a thiocarbonyl group;

each of R³ is independently C₁-C₆ alkyl, C₂-C₆ alkenyl, C₂-C₆ alkynyl, C₁-C₆ heteroalkyl, halo, cyano, —C(O)R^{A}, —C(O)OR^{B}, —OR^{B}, —N(R^{C})(R^{D}), —C(O)N(R^{C})(R^{D}), —N(R^{C})C(O)R^{A}, —S(O)ₓR^{E}, —S(O)ₓN(R^{C})(R^{D}), or —N(R^{C})S(O)ₓR^{E}, wherein each alkyl, alkenyl, alkynyl, and heteroalkyl is independently and optionally substituted with one or more R⁶;

each R⁴ is independently C₁-C₆ alkyl, C₂-C₆ alkenyl, C₂-C₆ alkynyl, C₁-C₆ heteroalkyl, halo, cyano, oxo, —C(O)R^{A}, —C(O)OR^{B}, —OR^{B}, —N(R^{C})(R^{D}), —C(O)N(R^{C})(R^{D}), —N(R^{C})C(O)R^{A}, —S(O)ₓR^{E}, —S(O)ₓN(R^{C})(R^{D}), —N(R^{C})S(O)ₓR^{E}, carbocyclyl, heterocyclyl, aryl, or heteroaryl, wherein each alkyl, alkenyl, alkynyl, heteroalkyl, carbocyclyl, heterocyclyl, aryl, and heteroaryl is independently and optionally substituted with one or more R⁷;

each of R^{A}, R^{B}, R^{C}, R^{D}, and R^{E} is independently hydrogen or C₁-C₆ alkyl;

each R⁶ is independently C₁-C₆ alkyl, oxo, cyano, —OR^{B}, —N(R^{C})(R^{D}), —C(O)N(R^{C})(R^{D}), —N(R^{C})C(O)R^{A}, aryl, or heteroaryl, wherein each aryl and heteroaryl is independently and optionally substituted with one or more R⁸;

each R⁷ is independently halo, oxo, cyano, —OR^{B}, —N(R^{C})(R^{D}), —C(O)N(R^{C})(R^{D}), or —N(R^{C})C(O)R^{A};

each R⁸ is independently C₁-C₆ alkyl, cyano, —OR^{B}, —N(R^{C})(R^{D}), —C(O)N(R^{C})(R^{D}), or —N(R^{C})C(O)R^{A};

n is 0, 1, 2, 3 or 4; and

x is 0, 1, or 2.

In some embodiments, the degradation compound is a compound of Formula (II) (COF2), wherein the COF2 is:

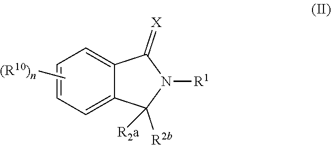

or a pharmaceutically acceptable salt, ester, hydrate, tautomer, or prodrug thereof, wherein:

X is O or S;

R¹ is C₁-C₆ alkyl, C₂-C₆ alkenyl, C₂-C₆ alkynyl, C₁-C₆ heteroalkyl, carbocyclyl, heterocyclyl, aryl, or heteroaryl, each of which is independently and optionally substituted by one or more R⁴;

each of R^{2a} and R^{2b} is independently hydrogen or C₁-C₆ alkyl; or R^{2a} and R^{2b} together with the carbon atom to which they are attached to form carbonyl group or thiocarbonyl group;

each of R¹⁰ is independently C₁-C₆ alkyl, C₂-C₆ alkenyl, C₂-C₆ alkynyl, C₁-C₆ heteroalkyl, halo, cyano, —C(O)R^{A}, —C(O)OR^{B}, —OR^{B}, —N(R^{C})(R^{D}), —C(O)N(R^{C})(R^{D}), —N(R^{C})C(O)R^{A}, —S(O)ₓR^{E}, —S(O)ₓN(R^{C})(R^{D}), or —N(R^{C})S(O)ₓR^{E}, or L-Tag; wherein each alkyl, alkenyl, alkynyl, and heteroalkyl is independently and optionally substituted with one or more R¹¹;

each R⁴ is independently C₁-C₆ alkyl, C₂-C₆ alkenyl, C₂-C₆ alkynyl, C₁-C₆ heteroalkyl, halo, cyano, oxo, C(O)R^{A}, —C(O)OR^{B}, OR^{B}, —N(R^{C})(R^{D}), —C(O)N(R^{C})(R^{D}), —N(R^{C})C(O)R^{A}, S(O)ₓR^{E}, —S(O)ₓN(R^{C})(R^{D}), —N(R^{C})S(O)ₓR^{E}, carbocyclyl, heterocyclyl, aryl, or heteroaryl, wherein each alkyl, alkenyl, alkynyl, heteroalkyl, carbocyclyl, heterocyclyl, aryl, and heteroaryl is independently and optionally substituted with one or more R⁷;

each of R^{A}, R^{B}, R^{C}, R^{D}, and R^{E} is independently hydrogen or C₁-C₆ alkyl;

each R¹¹ is independently C₁-C₆ alkyl, halo, oxo, cyano, —OR^{B}, —N(R^{C})(R^{D}), —C(O)N(R^{C})(R^{D}), —N(R^{C})C(O)R^{A}, aryl, or heteroaryl, wherein each aryl and heteroaryl is independently and optionally substituted with one or more R⁸;

each R⁷ is independently halo, oxo, cyano, —OR^{B}, —N(R^{C})(R^{D}), —C(O)N(R^{C})(RD), or —N(R^{C})C(O)R^{A};

each R⁸ is independently C₁-C₆ alkyl, halo, cyano, —OR^{B}, —N(R^{C})(R^{D}), —C(O)N(R^{C})(R^{D}), or —N(R^{C})C(O)R^{A};

each L is independently C₁-C₆ alkyl, C₂-C₆ alkenyl, C₂-C₆ alkynyl, C₁-C₆ heteroalkyl, —C(O)R^{A1}, —C(O)OR^{B1}, —OR^{B1}, —N(R^{C1})(R^{D1}), —C(O)N(R^{C1})(R^{D1}), —N(R^{C1})C(O)R^{A1}, —S(O)ₓR^{E1}, —S(O)ₓN(R^{C1})(R^{D1}), or —N(R^{C1})S(O)ₓR^{E1}, wherein each alkyl, alkenyl, alkynyl, and heteroalkyl is independently and optionally substituted with one or more R¹²;

each Tag is a targeting moiety capable of binding to a target protein;

each of R^{A1}, R^{B1}, R^{C1}, R^{D1}, and R^{E1} is independently hydrogen, C₁-C₆ alkyl, C₂-C₆ alkenyl, C₂-C₆ alkynyl, C₁-C₆ heteroalkyl, carbocyclyl, heterocyclyl, aryl, or heteroaryl, wherein each alkyl, alkenyl, alkynyl, heteroalkyl, carbocyclyl, heterocyclyl, aryl, and heteroaryl is independently and optionally substituted with one or more R¹²;

each R¹² is independently C₁-C₆ alkyl, halo, cyano, carbocyclyl, or heterocyclyl;

n is 0, 1, 2, 3 or 4; and

x is 0, 1, or 2.

In some embodiments, the degradation compound is a compound of Formula (III) (COF3), wherein the COF3 is:

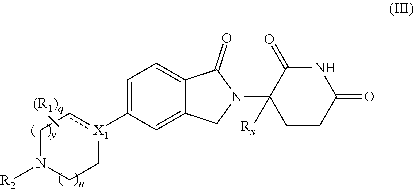

or a pharmaceutically acceptable salt, ester, hydrate, solvate, or tautomer thereof, wherein:

X₁ is CR₃;

[symbol] is optionally a double bond when X₁ is CR₃ and R₃ is absent;

each R₁ is independently C₁-C₆ alkyl, C₁-C₆ haloalkyl, C₁-C₆ hydroxyalkyl, or halo, or

two R₁ together with the carbon atoms to which they are attached form a 5- or 6-membered heterocyclyl ring, or

two R₁, when on adjacent atoms, together with the atoms to which they are attached form a C₆-C₁₀ aryl or 5- or 6-membered heteroaryl ring comprising 1 to 3 heteroatoms selected from O, N, and S;

R₂ is hydrogen, C₁-C₆ alkyl, —C(O)C₁-C₆ alkyl, —C(O)(CH₂)₀₋₃—C₆-C₁₀ aryl, —C(O)O(CH₂)₀₋₃—C₆-C₁₀aryl, C₆-C₁₀ aryl, or 5- or 6-membered heteroaryl comprising 1 to 3 heteroatoms selected from O, N, and S, C₃-C₈ carbocyclyl, or 5- to 7-heterocyclyl comprising 1 to 3 heteroatoms selected from O, N, and S, wherein the alkyl is optionally substituted with one or more R₄; and the aryl, heteroaryl, carbocyclyl, and heterocyclyl are optionally substituted with one or more R₅, or

R₁ and R₂, when on adjacent atoms, together with the atoms to which they are attached form a 5- or 6-membered heterocyclyl ring;

R₃ is hydrogen, or R₃ is absent when [symbol] is a double bond;

each R₄ is independently selected from —C(O)OR₆, —C(O)NR₆R₆, —NR₆C(O)R₆, halo, —OH, —NH₂, cyano, C₆-C₁₀ aryl, 5- or 6-membered heteroaryl comprising 1 to 4 heteroatoms selected from O, N, and S, C₃-C₈ carbocyclyl, and 5- to 7-membered heterocyclyl ring comprising 1 to 3 heteroatoms selected from O, N, and S, wherein the aryl, heteroaryl, carbocyclyl, and heterocyclyl are optionally substituted with one or more R₇;

each R₅ is independently selected from C₁-C₆ alkyl, C₂-C₆ alkenyl, C₂-C₆ alkynyl, C₁-C₆ alkoxy, C₁-C₆ haloalkyl, C₁-C₆ haloalkoxy, C₁-C₆ hydroxyalkyl, halo, —OH, —NH₂, cyano, C₃-C₇ carbocyclyl, 5- to 7-membered heterocyclyl comprising 1 to 3 heteroatoms selected from O, N, and S, C₆-C₁₀ aryl, and 5- or 6-membered heteroaryl comprising 1 to 3 heteroatoms selected from O, N, and S, or

two R₅, when on adjacent atoms, together with the atoms to which they are attached form a C₆-C₁₀ aryl or 5- or 6-membered heteroaryl comprising 1 to 3 heteroatoms selected from O, N, and S, optionally substituted with one or more R₁₀, or

two R5, when on adjacent atoms, together with the atoms to which they are attached form a C₅-C₇ carbocyclyl or 5- to 7-membered heterocyclyl comprising 1 to 3 heteroatoms selected from O, N, and S optionally substituted with one or more R₁₀;

R₆ and R₆ are each independently hydrogen, C₁-C₆ alkyl, or C₆-C₁₀ aryl;

each R₇ is independently selected from C₁-C₆ alkyl, C₂-C₆ alkenyl, C₂-C₆ alkynyl, C₁-C₆ alkoxy, C₁-C₆ haloalkyl, C₁-C₆ haloalkoxy, —C(O)R₈, —(CH₂)₀₋₃C(O)OR₈, —C(O)NR₈R₉, —NR₈C(O)R₉, —NR₈C(O)OR₉, —S(O)ₚNR₈R₉, —S(O)ₚR₁₂, (C₁-C₆)hydroxyalkyl, halo, —OH, —O(CH₂)₁₋₃CN, —NH₂, cyano, —O(CH₂)₀₋₃—C₆-C₁₀ aryl, adamantyl, —O(CH₂)₀₋₃-5- or 6-membered heteroaryl comprising 1 to 3 heteroatoms selected from O, N, and S, C₆-C₁₀ aryl, monocyclic or bicyclic 5- to 10-membered heteroaryl comprising 1 to 3 heteroatoms selected from O, N, and S, C₃-C₇ carbocyclyl, and 5- to 7-membered heterocyclyl comprising 1 to 3 heteroatoms selected from O, N, and S, wherein the alkyl is optionally substituted with one or more R₁₁, and the aryl, heteroaryl, and heterocyclyl are optionally substituted with one or more substituents each independently selected from halogen, C₁-C₆ alkyl, C₁-C₆ haloalkyl, and C₁-C₆ alkoxy, or

two R₇ together with the carbon atom to which they are attached form a ═(O), or

two R₇, when on adjacent atoms, together with the atoms to which they are attached form a C₆-C₁₀ aryl or 5- or 6-membered heteroaryl comprising 1 to 3 heteroatoms selected from O, N, and S, optionally substituted with one or more R₁₀, or

two R₇ together with the atoms to which they are attached form a C₅-C₇ carbocyclyl or a 5- to 7-membered heterocyclyl comprising 1 to 3 heteroatoms selected from O, N, and S, optionally substituted with one or more R₁₀;

R₈ and R₉ are each independently hydrogen or C₁-C₆ alkyl;

each R₁₀ is independently selected from C₁-C₆ alkyl, C₁-C₆ alkoxy, C₁-C₆ haloalkyl, C₁-C₆ haloalkoxy, C₁-C₆ hydroxyalkyl, halo, —OH, —NH₂, and cyano, or

two R₁₀ together with the carbon atom to which they are attached form a ═(O);

each R₁₁ is independently selected from cyano, C₁-C₆ alkoxy, C₆-C₁₀ aryl, and 5- to 7-membered heterocyclyl comprising 1 to 3 heteroatoms selected from O, N, and S, wherein each aryl and heterocyclyl is optionally substituted with one or more substituents each independently selected from C₁-C₆ alkyl, C₁-C₆ alkoxy, C₁-C₆ haloalkyl, C₁-C₆ haloalkoxy, C₁-C₆ hydroxyalkyl, halo, —OH, —NH₂, and cyano;

R₁₂ is C₁-C₆ alkyl, C₁-C₆ haloalkyl, C₆-C₁₀ aryl, or 5- to 7-membered heterocyclyl comprising 1 to 3 heteroatoms selected from O, N, and S;

Rₓ is hydrogen or deuterium;

p is 0, 1, or 2;

n is 0, 1, or 2;

y is 1 or 2, wherein n+y≤3; and

q is 0, 1, 2, 3, or 4.

Additional exemplary degradation compounds are disclosed in Table 29.

TABLE 29
Exemplary degradation compounds

| Cmpd No. | Compound Name |
|---|---|
| I-1 | 3-(5-(1-ethylpiperidin-4-yl)-1- |
| | oxoisoindolin-2-yl)piperidine- |
| | 2,6-dione |
| I-2 | 3-(1-oxo-5-(1-propylpiperidin-4- |
| | yl)isoindolin-2-yl)piperidine- |
| | 2,6-dione |
| I-3 | 3-(5-(1- |
| | (cyclopropylmethyl)piperidin-4- |
| | yl)-1-oxoisoindolin-2- |
| | yl)piperidine-2,6-dione |
| I-4 | 3-(5-(1-isobutylpiperidin-4-yl)- |
| | 1-oxoisoindolin-2-yl)piperidine- |
| | 2,6-dione |
| I-5 | 3-(5-(1- |
| | (cyclobutylmethyl)piperidin-4- |
| | yl)-1-oxoisoindolin-2-yl)piperidine- |
| | 2,6-dione |
| I-6 | 3-(5-(1-(oxazol-2- |
| | ylmethyl)piperidin-4-yl)-1- |
| | oxoisoindolin-2-yl)piperidine- |
| | 2,6-dione |
| I-7 | 3-(1-oxo-5-(1-(thiazol-2- |
| | ylmethyl)piperidin-4- |
| | yl)isoindolin-2-yl)piperidine- |
| | 2,6-dione |
| I-8 | 3-(5-(1- |
| | (cyclopentylmethyl)piperidin-4- |
| | yl)-1-oxoisoindolin-2- |
| | yl)piperidine-2,6-dione |
| I-9 | 3-(5-(1-((5-chlorothiophen-2- |
| | yl)methyl)piperidin-4-yl)-1- |
| | oxoisoindolin-2-yl)piperidine- |
| | 2,6-dione |
| I-10 | 3-(5-(1-((2-chlorothiazol-5- |
| | yl)methyl)piperidin-4-yl)-1- |
| | oxoisoindolin-2-yl)piperidine- |
| | 2,6-dione |
| I-11 | 3-(5-(1- |
| | (cyclohexylmethyl)piperidin-4- |
| | yl)-1-oxoisoindolin-2- |
| | yl)piperidine-2,6-dione |
| I-12 | 3-(1-oxo-5-(1-(2-(pyrrolidin-1- |
| | yl)ethyl)piperidin-4- |
| | yl)isoindolin-2-yl)piperidine- |
| | 2,6-dione |
| I-13 | 3-(1-oxo-5-(1-((tetrahydro-2H- |
| | pyran-4-yl)methyl)piperidin-4- |
| | yl)isoindolin-2-yl)piperidine- |
| | 2,6-dione |
| I-14 | 3-(1-oxo-5-(1- |
| | phenethylpiperidin-4- |
| | yl)isoindolin-2-yl)piperidine- |
| | 2,6-dione |
| I-15 | 3-(5-(1-(3- |
| | fluorobenzyl)piperidin-4-yl)-1- |
| | oxoisoindolin-2-yl)piperidine- |
| | 2,6-dione |
| I-16 | 3-(5-(1-(3- |
| | chlorobenzyl)piperidin-4-yl)-1- |
| | oxoisoindolin-2-yl)piperidine- |
| | 2,6-dione |
| I-17 | 3-(5-(1-(2- |
| | fluorobenzyl)piperidin-4-yl)-1- |
| | oxoisoindolin-2-yl)piperidine- |
| | 2,6-dione |
| I-18 | 3-(5-(1-(2- |
| | chlorobenzyl)piperidin-4-yl)-1- |
| | oxoisoindolin-2-yl)piperidine- |
| | 2,6-dione |
| I-19 | 3-(1-oxo-5-(1-(2-(piperidin-1- |
| | yl)ethyl)piperidin-4- |
| | yl)isoindolin-2-yl)piperidine- |
| | 2,6-dione |
| I-20 | 3-(5-(1-((3,5-dimethylisoxazol- |
| | 4-yl)methyl)piperidin-4-yl)-1- |
| | oxoisoindolin-2-yl)piperidine- |
| | 2,6-dione |
| I-21 | 3-(5-(1-((1,3-dimethyl-1H- |
| | pyrazol-5-yl)methyl)piperidin-4- |
| | yl)-1-oxoisoindolin-2- |
| | yl)piperidine-2,6-dione |
| I-22 | 3-(5-(1-((6-methylpyridin-2- |
| | yl)methyl)piperidin-4-yl)-1- |
| | oxoisoindolin-2-yl)piperidine- |
| | 2,6-dione |
| I-23 | 3-(5-(1-(3- |
| | morpholinopropyl)piperidin-4- |
| | yl)-1-oxoisoindolin-2- |
| | yl)piperidine-2,6-dione |
| I-24 | 3-(5-(1-(2,6- |
| | difluorobenzyl)piperidin-4-yl)-1- |
| | oxoisoindolin-2-yl)piperidine- |
| | 2,6-dione |
| I-25 | 3-(5-(1-(2,6- |
| | dichlorobenzyl)piperidin-4-yl)- |
| | 1-oxoisoindolin-2-yl)piperidine- |
| | 2,6-dione |
| I-26 | 3-(5-(1-(3,5- |
| | difluorobenzyl)piperidin-4-yl)-1- |
| | oxoisoindolin-2-yl)piperidine- |
| | 2,6-dione |
| I-27 | 3-(5-(1-(3,5- |
| | dibromobenzyl)piperidin-4-yl)- |
| | 1-oxoisoindolin-2-yl)piperidine- |
| | 2,6-dione |
| I-28 | 3-(5-(1-(3-chloro-5- |
| | fluorobenzyl)piperidin-4-yl)-1- |
| | oxoisoindolin-2-yl)piperidine- |
| | 2,6-dione |
| I-29 | 3-(5-(1-(2,5- |
| | difluorobenzyl)piperidin-4-yl)-1- |
| | oxoisoindolin-2-yl)piperidine- |
| | 2,6-dione |
| I-30 | 3-(5-(1-(2,5- |
| | dichlorobenzyl)piperidin-4-yl)- |
| | 1-oxoisoindolin-2-yl)piperidine- |
| | 2,6-dione |
| I-31 | 4-((4-(2-(2,6-dioxopiperidin-3- |
| | yl)-1-oxoisoindolin-5- |
| | yl)piperidin-1- |
| | yl)methyl)benzonitrile |
| | (or 3-(5-(1-(4- |
| | nitrilebenzyl)piperidin-4-yl)-1- |
| | oxoisoindolin-2-yl)piperidine- |
| | 2,6-dione) |
| I-32 | 3-(5-(1-(4- |
| | (hydroxymethyl)benzyl)piperidin- |
| | 4-yl)-1-oxoisoindolin-2- |
| | yl)piperidine-2,6-dione |
| I-33 | 3-(5-(1-(3,4- |
| | dichlorobenzyl)piperidin-4-yl)- |
| | 1-oxoisoindolin-2-yl)piperidine- |
| | 2,6-dione |
| I-34 | 3-(5-(1-(4-chloro-2- |
| | fluorobenzyl)piperidin-4-yl)-1- |
| | oxoisoindolin-2-yl)piperidine- |
| | 2,6-dione |
| I-35 | 3-(5-(1-(2-chloro-4- |
| | fluorobenzyl)piperidin-4-yl)-1- |
| | oxoisoindolin-2-yl)piperidine- |
| | 2,6-dione |
| I-36 | 3-((4-(2-(2,6-dioxopiperidin-3- |
| | yl)-1-oxoisoindolin-5- |
| | yl)piperidin-1- |
| | yl)methyl)benzonitrile |
| I-37 | 3-(5-(1-(2,3- |
| | difluorobenzyl)piperidin-4-yl)-1- |
| | oxoisoindolin-2-yl)piperidine- |
| | 2,6-dione |
| I-38 | 2-((4-(2-(2,6-dioxopiperidin-3- |
| | yl)-1-oxoisoindolin-5- |
| | yl)piperidin-1- |
| | yl)methyl)benzonitrile |
| I-39 | 3-(5-(1-(4- |
| | methoxybenzyl)piperidin-4-yl)- |
| | 1-oxoisoindolin-2-yl)piperidine- |
| | 2,6-dione |
| I-40 | 3-(5-(1-(2,5- |
| | dimethylbenzyl)piperidin-4-yl)- |
| | 1-oxoisoindolin-2-yl)piperidine- |
| | 2,6-dione |
| I-41 | 3-(5-(1-(3,4- |
| | dimethylbenzyl)piperidin-4-yl)- |
| | 1-oxoisoindolin-2-yl)piperidine- |
| | 2,6-dione |
| I-42 | 3-(5-(1-(2,4- |
| | dimethylbenzyl)piperidin-4-yl)- |
| | 1-oxoisoindolin-2-yl)piperidine- |
| | 2,6-dione |
| I-43 | 3-(5-(1-((1H-indazol-4- |
| | yl)methyl)piperidin-4-yl)-1- |
| | oxoisoindolin-2-yl)piperidine- |
| | 2,6-dione |
| I-44 | 3-(5-(1-((1H-benzo[d]imidazol- |
| | 2-yl)methyl)piperidin-4-yl)-1- |
| | oxoisoindolin-2-yl)piperidine- |
| | 2,6-dione |
| I-45 | 3-(5-(1-(4- |
| | isopropylbenzyl)piperidin-4-yl)- |
| | 1-oxoisoindolin-2-yl)piperidine- |
| | 2,6-dione |
| I-46 | methyl 5-((4-(2-(2,6- |
| | dioxopiperidin-3-yl)-1- |
| | oxoisoindolin-5-yl)piperidin-1- |
| | yl)methyl)furan-2-carboxylate |
| I-47 | 3-(5-(1-(naphthalen-2- |
| | ylmethyl)piperidin-4-yl)-1- |
| | oxoisoindolin-2-yl)piperidine- |
| | 2,6-dione |
| I-48 | 3-(1-oxo-5-(1-(quinolin-2- |
| | ylmethyl)piperidin-4- |
| | yl)isoindolin-2-yl)piperidine- |
| | 2,6-dione |
| I-49 | 3-(5-(1-(naphthalen-1- |
| | ylmethyl)piperidin-4-yl)-1- |
| | oxoisoindolin-2-yl)piperidine- |
| | 2,6-dione |
| I-50 | 3-(5-(1-((1-methyl-1H- |
| | benzo[d]imidazol-2- |
| | yl)methyl)piperidin-4-yl)-1- |
| | oxoisoindolin-2-yl)piperidine- |
| | 2,6-dione |
| I-51 | 3-(1-oxo-5-(1-(4- |
| | (trifluoromethoxy)benzyl)piperidin- |
| | 4-yl)isoindolin-2- |
| | yl)piperidine-2,6-dione |
| I-52 | 3-(5-(1-(4-(1H-pyrrol-1- |
| | yl)benzyl)piperidin-4-yl)-1- |
| | oxoisoindolin-2-yl)piperidine- |
| | 2,6-dione |
| I-53 | 3-(5-(1-(4-(1H-1,2,4-triazol-1- |
| | yl)benzyl)piperidin-4-yl)-1- |
| | oxoisoindolin-2-yl)piperidine- |
| | 2,6-dione |
| I-54 | 3-(1-oxo-5-(1-(3- |
| | (trifluoromethoxy)benzyl)piperidin- |
| | 4-yl)isoindolin-2- |
| | yl)piperidine-2,6-dione |
| I-55 | 3-(1-oxo-5-(1-(2- |
| | (trifluoromethoxy)benzyl)piperidin- |
| | 4-yl)isoindolin-2- |
| | yl)piperidine-2,6-dione |
| I-56 | 3-(1-oxo-5-(1-((3-phenyl-1,2,4- |
| | oxadiazol-5-yl)methyl)piperidin- |
| | 4-yl)isoindolin-2-yl)piperidine- |
| | 2,6-dione |
| I-57 | 3-(5-(1-benzyipiperidin-4-yl)-1- |
| | oxoisoindolin-2-yl)piperidine- |
| | 2,6-dione |
| I-58 | 3-(1-oxo-5-(1-(pyridin-2- |
| | ylmethyl)piperidin-4- |
| | yl)isoindolin-2-yl)piperidine- |
| | 2,6-dione |
| I-59 | 3-(1-oxo-5-(1-(pyridin-3- |
| | ylmethyl)piperidin-4- |
| | yl)isoindolin-2-yl)piperidine- |
| | 2,6-dione |
| I-60 | 3-(1-oxo-5-(1-(pyridin-4- |
| | ylmethyl)piperidin-4- |
| | yl)isoindolin-2-yl)piperidine- |
| | 2,6-dione |
| I-61 | 3-(1-oxo-5-(1-(pyrimidin-5- |
| | ylmethyl)piperidin-4- |
| | yl)isoindolin-2-yl)piperidine- |
| | 2,6-dione |
| I-62 | 3-(1-oxo-5-(1-(1- |
| | phenylethyl)piperidin-4- |
| | yl)isoindolin-2-yl)piperidine- |
| | 2,6-dione |
| I-63 | 3-(5-(1-(4- |
| | (fluoromethyl)benzyl)piperidin- |
| | 4-yl)-1-oxoisoindolin-2- |
| | yl)piperidine-2,6-dione |
| I-64 | 3-(5-(1-(3,4- |
| | difluorobenzyl)piperidin-4-yl)-1- |
| | oxoisoindolin-2-yl)piperidine- |
| | 2.6-dione |
| I-65 | 2-((4-(2-(2,6-dioxopiperidin-3- |
| | yl)-1-oxoisoindolin-5- |
| | yl)piperidin-1- |
| | yl)methyl)pyrimidine-5- |
| | carbonitrile |
| I-66 | 3-(5-(1-(4-ethylbenzyl)piperidin- |
| | 4-yl)-1-oxoisoindolin-2- |
| | yl)piperidine-2,6-dione |
| I-67 | 3-(5-(1-(2- |
| | methoxybenzyl)piperidin-4-yl)- |
| | 1-oxoisoindolin-2-yl)piperidine- |
| | 2,6-dione |
| I-68 | 3-(5-(1-((2-methoxypyrimidin-5- |
| | yl)methyl)piperidin-4-yl)-1- |
| | oxoisoindolin-2-yl)piperidine- |
| | 2,6-dione |
| I-69 | 3-(5-(1-(3-fluoro-4- |
| | methylbenzyl)piperidin-4-yl)-1- |
| | oxoisoindolin-2-yl)piperidine- |
| | 2.6-dione |
| I-70 | 3-(5-(1-(4- |
| | (difluoromethyl)benzyl)piperidin- |
| | 4-yl)-1-oxoisoindolin-2- |
| | yl)piperidine-2,6-dione |
| I-71 | 4-((4-(2-(2,6-dioxopiperidin-3- |
| | yl)-1-oxoisoindolin-5- |
| | yl)piperidin-1- |
| | yl)methyl)benzamide |
| I-72 | 4-((4-(2-(2,6-dioxopiperidin-3- |
| | yl)-1-oxoisoindolin-5- |
| | yl)piperidin-1-yl)methyl)benzoic |
| | acid |
| I-73 | 3-(5-(1-(3- |
| | (difluoromethyl)benzyl)piperidin- |
| | 4-yl)-1-oxoisoindolin-2- |
| | yl)piperidine-2,6-dione |
| I-74 | 3-((4-(2-(2,6-dioxopiperidin-3- |
| | yl)-1-oxoisoindolin-5- |
| | yl)piperidin-1-yl)methyl)benzoic |
| | acid |
| I-75 | 3-(1-oxo-5-(1-(4- |
| | propylbenzyl)piperidin-4- |
| | yl)isoindolin-2-yl)piperidine- |
| | 2,6-dione |
| I-76 | 3-(1-oxo-5-(1-(4- |
| | (trifluoromethyl)benzyl)piperidin- |
| | 4-yl)isoindolin-2- |
| | yl)piperidine-2,6-dione |
| I-77 | 3-(5-(1-(4- |
| | (difluoromethoxy)benzyl)piperidin- |
| | 4-yl)-1-oxoisoindolin-2- |
| | yl)piperidine-2,6-dione |
| I-78 | 3-(1-oxo-5-(1-((5- |
| | (trifluoromethyl)pyridin-2- |
| | yl)methyl)piperidin-4- |
| | yl)isoindolin-2-yl)piperidine- |
| | 2,6-dione |
| I-79 | 3-(5-(1-(3- |
| | (difluoromethoxy)benzyl)piperidin- |
| | 4-yl)-1-oxoisoindolin-2- |
| | yl)piperidine-2,6-dione |
| I-80 | 3-(5-(1-(2- |
| | (difluoromethoxy)benzyl)piperidin- |
| | 4-yl)-1-oxoisoindolin-2- |
| | yl)piperidine-2,6-dione |
| I-81 | 3-(5-(1-(4- |
| | cyclobutylbenzyl)piperidin-4- |
| | yl)-1-oxoisoindolin-2- |
| | yl)piperidine-2,6-dione |
| I-82 | 3-(5-(1-((2,3- |
| | dihydrobenzo[b][1,4]dioxin-5- |
| | yl)methyl)piperidin-4-yl)-1- |
| | oxoisoindolin-2-yl)piperidine- |
| | 2,6-dione |
| I-83 | 3-(5-(1-((2,3- |
| | dihydrobenzo[b][1,4]dioxin-6- |
| | yl)methyl)piperidin-4-yl)-1- |
| | oxoisoindolin-2-yl)piperidine- |
| | 2,6-dione |
| I-84 | 3-(5-(1-(4-(tert- |
| | butyl)benzyl)piperidin-4-yl)-1- |
| | oxoisoindolin-2-yl)piperidine- |
| | 2,6-dione |
| I-85 | 3-(5-(1-(4- |
| | isobutylbenzyl)piperidin-4-yl)-1- |
| | oxoisoindolin-2-yl)piperidine- |
| | 2,6-dione |
| I-86 | N-(4-((4-(2-(2,6-dioxopiperidin- |
| | 3-yl)-1-oxoisoindolin-5- |
| | yl)piperidin-1- |
| | yl)methyl)phenyl)acetamide |
| I-87 | 3-(5-(1-((2,2- |
| | difluorobenzo[d][1,3]dioxol-5- |
| | yl)methyl)piperidin-4-yl)-1- |
| | oxoisoindolin-2-yl)piperidine- |
| | 2,6-dione |
| I-88 | 3-(5-(1-((3,4-dihydro-2H- |
| | benzo[b][1,4]dioxepin-7- |
| | yl)methyl)piperidin-4-yl)-1- |
| | oxoisoindolin-2-yl)piperidine- |
| | 2,6-dione |
| I-89 | 3-(1-oxo-5-(1-(4-(tert- |
| | pentyl)benzyl)piperidin-4- |
| | yl)isoindolin-2-yl)piperidine- |
| | 2,6-dione |
| I-90 | 3-(5-(1-([1,1′-biphenyl]-4- |
| | ylmethyl)piperidin-4-yl)-1- |
| | oxoisoindolin-2-yl)piperidine- |
| | 2,6-dione |
| I-91 | 3-(5-(1-(4-(1H-pyrazol-1- |
| | yl)benzyl)piperidin-4-yl)-1- |
| | oxoisoindolin-2-yl)piperidine- |
| | 2,6-dione |
| I-92 | 3-(5-(1-(4-(1H-imidazol-1- |
| | yl)benzyl)piperidin-4-yl)-1- |
| | oxoisoindolin-2-yl)piperidine- |
| | 2,6-dione |
| I-93 | 3-(5-(1-(3-(1H-pyrazol-1- |
| | yl)benzyl)piperidin-4-yl)-1- |
| | oxoisoindolin-2-yl)piperidine- |
| | 2,6-dione |
| I-94 | 3-(5-(1-(4- |
| | cyclohexylbenzyl)piperidin-4- |
| | yl)-1-oxoisoindolin-2- |
| | yl)piperidine-2,6-dione |
| I-95 | 3-(1-oxo-5-(1-(pyrimidin-2- |
| | ylmethyl)piperidin-4- |
| | yl)isoindolin-2-yl)piperidine- |
| | 2,6-dione |
| I-96 | 3-(5-(1-(4- |
| | bromobenzyl)piperidin-4-yl)-1- |
| | oxoisoindolin-2-yl)piperidine- |
| | 2,6-dione |
| I-97 | 3-(5-(1-(4- |
| | chlorobenzyl)piperidin-4-yl)-1- |
| | oxoisoindolin-2-yl)piperidine- |
| | 2,6-dione |
| I-98 | 3-(5-(1-(3,5- |
| | dichlorobenzyl)piperidin-4-yl)- |
| | 1-oxoisoindolin-2-yl)piperidine- |
| | 2,6-dione |
| I-99 | 3-(5-(1-(4-chloro-3- |
| | fluorobenzyl)piperidin-4-yl)-1- |
| | oxoisoindolin-2-yl)piperidine- |
| | 2,6-dione |
| I-100 | 3-(5-(1-(3-chloro-4- |
| | fluorobenzyl)piperidin-4-yl)-1- |
| | oxoisoindolin-2-yl)piperidine- |
| | 2,6-dione |
| I-101 | 3-(5-(1-(2,4- |
| | difluorobenzyl)piperidin-4-yl)-1- |
| | oxoisoindolin-2-yl)piperidine- |
| | 2,6-dione |
| I-102 | 3-(5-(1-(3- |
| | methoxybenzyl)piperidin-4-yl)- |
| | 1-oxoisoindolin-2-yl)piperidine- |
| | 2,6-dione |
| I-103 | 3-(5-(1- |
| | (benzo[c][1,2,5]oxadiazol-5- |
| | ylmethyl)piperidin-4-yl)-1- |
| | oxoisoindolin-2-yl)piperidine- |
| | 2,6-dione |
| I-104 | 3-(5-(1-(2- |
| | cyclopropylbenzyl)piperidin-4- |
| | yl)-1-oxoisoindolin-2- |
| | yl)piperidine-2,6-dione |
| I-105 | 3-(5-(1-((1,3- |
| | dihydroisobenzofuran-5- |
| | yl)methyl)piperidin-4-yl)-1- |
| | oxoisoindolin-2-yl)piperidine- |
| | 2,6-dione |
| I-106 | 3-(1-oxo-5-(1-(2- |
| | (trifluoromethyl)benzyl)piperidin- |
| | 4-yl)isoindolin-2- |
| | yl)piperidine-2,6-dione |
| I-107 | 3-(5-(1-(3-(tert- |
| | butyl)benzyl)piperidin-4-yl)-1- |
| | oxoisoindolin-2-yl)piperidine- |
| | 2,6-dione |
| I-108 | 3-(5-(1-(3- |
| | isopropoxybenzyl)piperidin-4- |
| | yl)-1-oxoisoindolin-2- |
| | yl)piperidine-2,6-dione |
| I-109 | 3-(1-oxo-5-(1-(4-(thiophen-3- |
| | yl)benzyl)piperidin-4- |
| | yl)isoindolin-2-yl)piperidine- |
| | 2,6-dione |
| I-110 | 3-(5-(1-(4- |
| | cyclopentylbenzyl)piperidin-4- |
| | yl)-1-oxoisoindolin-2- |
| | yl)piperidine-2,6-dione |
| I-111 | 3-(1-oxo-5-(1-(4-(pyrrolidin-1- |
| | yl)benzyl)piperidin-4- |
| | yl)isoindolin-2-yl)piperidine- |
| | 2,6-dione |
| I-112 | 3-(5-(1-(4- |
| | fluorobenzyl)piperidin-4-yl)-1- |
| | oxoisoindolin-2-yl)piperidine- |
| | 2,6-dione |
| I-113 | 3-(5-(1-(2,4- |
| | dichlorobenzyl)piperidin-4-yl)- |
| | 1-oxoisoindolin-2-yl)piperidine- |
| | 2,6-dione |
| I-114 | 3-(1-oxo-5-(1-(quinolin-8- |
| | ylmethyl)piperidin-4- |
| | yl)isoindolin-2-yl)piperidine- |
| | 2,6-dione |
| I-115 | 3-(5-(1-((1-methyl-1H-pyrazol- |
| | 4-yl)methyl)piperidin-4-yl)-1- |
| | oxoisoindolin-2-yl)piperidine- |
| | 2,6-dione |
| I-116 | 3-(5-(1-((1H-pyrazol-4- |
| | yl)methyl)piperidin-4-yl)-1- |
| | oxoisoindolin-2-yl)piperidine- |
| | 2,6-dione |
| I-117 | 3-(5-(1-((1-methyl-1H-pyrazol- |
| | 3-yl)methyl)piperidin-4-yl)-1- |
| | oxoisoindolin-2-yl)piperidine- |
| | 2,6-dione |
| I-118 | 3-(5-(1-((1H-pyrazol-3- |
| | yl)methyl)piperidin-4-yl)-1- |
| | oxoisoindolin-2-yl)piperidine- |
| | 2,6-dione |
| I-119 | 3-(5-(1-((1H-pyrrol-3- |
| | yl)methyl)piperidin-4-yl)-1- |
| | oxoisoindolin-2-yl)piperidine- |
| | 2,6-dione |
| I-120 | 3-(5-(1-((1H-imidazol-5- |
| | yl)methyl)piperidin-4-yl)-1- |
| | oxoisoindolin-2-yl)piperidine- |
| | 2,6-dione |
| I-121 | 3-(5-(1-((1-ethyl-1H-pyrazol-3- |
| | yl)methyl)piperidin-4-yl)-1- |
| | oxoisoindolin-2-yl)piperidine- |
| | 2,6-dione |
| I-122 | 3-(5-(1-((2-aminopyrimidin-5- |
| | yl)methyl)piperidin-4-yl)-1- |
| | oxoisoindolin-2-yl)piperidine- |
| | 2,6-dione |
| I-123 | 3-(5-(1-((6-aminopyridin-3- |
| | yl)methyl)piperidin-4-yl)-1- |
| | oxoisoindolin-2-yl)piperidine- |
| | 2,6-dione |
| I-124 | 3-(5-(1-((5-amino-1-methyl-1H- |
| | pyrazol-4-yl)methyl)piperidin-4- |
| | yl)-1-oxoisoindolin-2- |
| | yl)piperidine-2,6-dione |
| I-125 | 3-(5-(1-((6-methylimidazo[2,1- |
| | b]thiazol-5-yl)methyl)piperidin- |
| | 4-yl)-1-oxoisoindolin-2- |
| | yl)piperidine-2,6-dione |
| I-126 | 3-(5-(1-(imidazo[1,2-a]pyrazin- |
| | 3-ylmethyl)piperidin-4-yl)-1- |
| | oxoisoindolin-2-yl)piperidine- |
| | 2,6-dione |
| I-127 | 3-(5-(1-([1,2,4]triazolo[1,5- |
| | a]pyridin-5-ylmethyl)piperidin- |
| | 4-yl)-1-oxoisoindolin-2- |
| | yl)piperidine-2,6-dione |
| I-128 | 3-(1-oxo-5-(1-(pyrazolo[1,5- |
| | a]pyridin-4-ylmethyl)piperidin- |
| | 4-yl)isoindolin-2-yl)piperidine- |
| | 2,6-dione |
| I-129 | 3-(5-(1-((1,4-dimethyl-1H- |
| | imidazol-2-yl)methyl)piperidin- |
| | 4-yl)-1-oxoisoindolin-2- |
| | yl)piperidine-2,6-dione |
| I-130 | 3-(5-(1-(benzo[d]thiazol-5- |
| | ylmethyl)piperidin-4-yl)-1- |
| | oxoisoindolin-2-yl)piperidine- |
| | 2,6-dione |
| I-131 | 3-(1-oxo-5-(1-(pyrazolo[1,5- |
| | a]pyrimidin-6- |
| | ylmethyl)piperidin-4- |
| | yl)isoindolin-2-yl)piperidine- |
| | 2,6-dione |
| I-132 | 3-(5-(1-(imidazo[1,2- |
| | a]pyrimidin-3- |
| | ylmethyl)piperidin-4-yl)-1- |
| | oxoisoindolin-2-yl)piperidine- |
| | 2,6-dione |
| I-133 | 3-(5-(1-(imidazo[1,2- |
| | a]pyrimidin-2- |
| | ylmethyl)piperidin-4-yl)-1- |
| | oxoisoindolin-2-yl)piperidine- |
| | 2,6-dione |
| I-134 | 3-(5-(1-((1-cyclobutyl-1H-1,2,3- |
| | triazol-4-yl)methyl)piperidin-4- |
| | yl)-1-oxoisoindolin-2- |
| | yl)piperidine-2,6-dione |
| I-135 | 3-(1-oxo-5-(1-((4,5,6,7- |
| | tetrahydropyrazolo[1,5- |
| | a]pyridin-2-yl)methyl)piperidin- |
| | 4-yl)isoindolin-2-yl)piperidine- |
| | 2,6-dione |
| I-136 | 3-(5-(1-((1H-indol-2- |
| | yl)methyl)piperidin-4-yl)-1- |
| | oxoisoindolin-2-yl)piperidine- |
| | 2,6-dione |
| I-137 | 3-(5-(1-((1H-indazol-6- |
| | yl)methyl)piperidin-4-yl)-1- |
| | oxoisoindolin-2-yl)piperidine- |
| | 2,6-dione |
| I-138 | 3-(5-(1-((1H-pyrrolo[2,3- |
| | b]pyridin-3-yl)methyl)piperidin- |
| | 4-yl)-1-oxoisoindolin-2- |
| | yl)piperidine-2,6-dione |
| I-139 | 3-((4-(2-(2,6-dioxopiperidin-3- |
| | yl)-1-oxoisoindolin-5- |
| | yl)piperidin-1- |
| | yl)methyl)benzamide |
| I-140 | 3-(5-(1-((1H-pyrrolo[2,3- |
| | b]pyridin-6-yl)methyl)piperidin- |
| | 4-yl)-1-oxoisoindolin-2- |
| | yl)piperidine-2,6-dione |
| I-141 | 3-(5-(1-((3,4-dihydro-2H- |
| | benzo[b][1,4]thiazin-6- |
| | yl)methyl)piperidin-4-yl)-1- |
| | oxoisoindolin-2-yl)piperidine- |
| | 2,6-dione |
| I-142 | 3-(1-oxo-5-(1-((2-(pyrrolidin-1- |
| | yl)pyrimidin-5- |
| | yl)methyl)piperidin-4- |
| | yl)isoindolin-2-yl)piperidine- |
| | 2,6-dione |
| I-143 | 3-(5-(1-((2-(tert-butyl)thiazol-4- |
| | yl)methyl)piperidin-4-yl)-1- |
| | oxoisoindolin-2-yl)piperidine- |
| | 2,6-dione |
| I-144 | 3-(1-oxo-5-(1-((2-(thiophen-2- |
| | yl)thiazol-5-yl)methyl)piperidin- |
| | 4-yl)isoindolin-2-yl)piperidine- |
| | 2,6-dione |
| I-145 | 3-(5-(1-((2-cyclohexylthiazol-5- |
| | yl)methyl)piperidin-4-yl)-1- |
| | oxoisoindolin-2-yl)piperidine- |
| | 2,6-dione |
| I-146 | 3-(5-(1-((5-cyclopropyl-1H- |
| | pyrazol-3-yl)methyl)piperidin-4- |
| | yl)-1-oxoisoindolin-2- |
| | yl)piperidine-2,6-dione |
| I-147 | 3-(5-(1-((2- |
| | morpholinopyrimidin-5- |
| | yl)methyl)piperidin-4-yl)-1- |
| | oxoisoindolin-2-yl)piperidine- |
| | 2,6-dione |
| I-148 | 3-(1-oxo-5-(1-((3-phenyl-1H- |
| | pyrazol-4-yl)methyl)piperidin-4- |
| | yl)isoindolin-2-yl)piperidine- |
| | 2,6-dione |
| I-149 | 3-(5-(1-((6-methyl-1H-indol-3- |
| | yl)methyl)piperidin-4-yl)-1- |
| | oxoisoindolin-2-yl)piperidine- |
| | 2,6-dione |
| I-150 | methyl 4-((4-(2-(2,6- |
| | dioxopiperidin-3-yl)-1- |
| | oxoisoindolin-5-yl)piperidin-1- |
| | yl)methyl)-1H-pyrrole-2- |
| | carboxylate |
| I-151 | 3-(1-oxo-5-(1-((3-(pyridin-3-yl)- |
| | 1H-pyrazol-4- |
| | yl)methyl)piperidin-4- |
| | yl)isoindolin-2-yl)piperidine- |
| | 2,6-dione |
| I-152 | 3-(1-oxo-5-(1-((2-phenyl-1H- |
| | imidazol-4-yl)methyl)piperidin- |
| | 4-yl)isoindolin-2-yl)piperidine- |
| | 2,6-dione |
| I-153 | 3-(1-oxo-5-(1-((5-(pyridin-2-yl)- |
| | 1H-pyrazol-3- |
| | yl)methyl)piperidin-4- |
| | yl)isoindolin-2-yl)piperidine- |
| | 2,6-dione |
| I-154 | 3-(1-oxo-5-(1-((4-phenyl-1H- |
| | imidazol-2-yl)methyl)piperidin- |
| | 4-yl)isoindolin-2-yl)piperidine- |
| | 2,6-dione |
| I-155 | 3-(1-oxo-5-(piperidin-4- |
| | yl)isoindolin-2-yl)piperidine- |
| | 2,6-dione |
| I-156 | 3-(5-(1-(3,5-difluoro-4- |
| | hydroxybenzyl)piperidin-4-yl)- |
| | 1-oxoisoindolin-2-yl)piperidine- |
| | 2,6-dione |
| I-157 | 3-(5-(1-(2- |
| | methylbenzyl)piperidin-4-yl)-1- |
| | oxoisoindolin-2-yl)piperidine- |
| | 2,6-dione |
| I-158 | 3-(5-(1-(4- |
| | methylbenzyl)piperidin-4-yl)-1- |
| | oxoisoindolin-2-yl)piperidine- |
| | 2,6-dione |
| I-159 | 3-(5-(1-(3,5- |
| | dimethylbenzyl)piperidin-4-yl)- |
| | 1-oxoisoindolin-2-yl)piperidine- |
| | 2,6-dione |
| I-160 | 3-(5-((2S)-1-benzyl-2- |
| | methylpiperidin-4-yl)-1- |
| | oxoisoindolin-2-yl)piperidine- |
| | 2,6-dione |
| I-161 | 3-(5-((2R)-1-benzyl-2- |
| | methylpiperidin-4-yl)-1- |
| | oxoisoindolin-2-yl)piperidine- |
| | 2,6-dione |
| I-162 | 3-(5-(1-benzyl-2- |
| | methylpiperidin-4-yl)-1- |
| | oxoisoindolin-2-yl)piperidine- |
| | 2,6-dione |
| I-163 | 3-(5-(1-methyl-1,2,3,6- |
| | tetrahydropyridin-4-yl)-1- |
| | oxoisoindolin-2-yl)piperidine- |
| | 2,6-dione |
| I-164 | 3-(1-oxo-5-(1-((5,6,7,8- |
| | tetrahydronaphthalen-1- |
| | yl)methyl)piperidin-4- |
| | yl)isoindolin-2-yl)piperidine- |
| | 2,6-dione |
| I-165 | 3-(5-(azepan-4-yl)-1- |
| | oxoisoindolin-2-yl)piperidine- |
| | 2,6-dione |
| I-166 | 3-(5-((R)-azepan-4-yl)-1- |
| | oxoisoindolin-2-yl)piperidine- |
| | 2,6-dione |
| I-167 | 3-(5-((S)-azepan-4-yl)-1- |
| | oxoisoindolin-2-yl)piperidine- |
| | 2,6-dione |
| I-168 | 3-(1-oxo-5-(1-((l,2,3,4- |
| | tetrahydronaphthalen-1- |
| | yl)methyl)piperidin-4- |
| | yl)isoindolin-2-yl)piperidine- |
| | 2,6-dione |
| I-169 | methyl 2-(4-(2-(2,6- |
| | dioxopiperidin-3-yl)-1- |
| | oxoisoindolin-5-yl)piperidin-1- |
| | yl)acetate |
| I-170 | 3-(1-oxo-5-(1-phenylpiperidin-4- |
| | yl)isoindolin-2-yl)piperidine- |
| | 2,6-dione |
| I-171 | 3-(1-oxo-5-(2,2,6,6- |
| | tetramethylpiperidin-4- |
| | yl)isoindolin-2-yl)piperidine- |
| | 2,6-dione |
| I-172 | 3-(5-(1-benzyl-1,2,3,6- |
| | tetrahydropyridin-4-yl)-1- |
| | oxoisoindolin-2-yl)piperidine- |
| | 2,6-dione |
| I-173 | 3-(5-(1-(3- |
| | methylbenzyl)piperidin-4-yl)-1- |
| | oxoisoindolin-2-yl)piperidine- |
| | 2,6-dione |
| I-174 | 3-(5-(1-(2,6- |
| | dimethylbenzyl)piperidin-4-yl)- |
| | 1-oxoisoindolin-2-yl)piperidine- |
| | 2,6-dione |
| I-175 | 3-(1-oxo-5-(1-((5,6,7,8- |
| | tetrahydronaphthalen-2- |
| | yl)methyl)piperidin-4- |
| | yl)isoindolin-2-yl)piperidine- |
| | 2,6-dione |
| I-176 | ethyl 2-(4-(2-(2,6- |
| | dioxopiperidin-3-yl)-1- |
| | oxoisoindolin-5-yl)piperidin-1- |
| | yl)acetate |
| I-177 | tert-butyl 2-(4-(2-(2,6- |
| | dioxopiperidin-3-yl)-1- |
| | oxoisoindolin-5-yl)piperidin-1- |
| | yl)acetate |
| I-178 | 2-(4-(2-(2,6-dioxopiperidin-3- |
| | yl)-1-oxoisoindolin-5- |
| | yl)piperidin-1-yl)acetic acid |
| I-179 | 3-(1-oxo-5-(1-(3,3,3- |
| | trifluoropropyl)piperidin-4- |
| | yl)isoindolin-2-yl)piperidine- |
| | 2,6-dione |
| I-180 | 2-(4-(2-(2,6-dioxopiperidin-3- |
| | yl)-1-oxoisoindolin-5- |
| | yl)piperidin-1-yl)-N- |
| | phenylacetamide |
| I-181 | 3-(5-(1-(3- |
| | fluoropropyl)piperidin-4-yl)-1- |
| | oxoisoindolin-2-yl)piperidine- |
| | 2,6-dione |
| I-182 | tert-butyl 4-((4-(2-(2,6- |
| | dioxopiperidin-3-yl)-1- |
| | oxoisoindolin-5-yl)piperidin-1- |
| | yl)methyl)benzoate |
| I-183 | 3-(5-(2-methylpiperidin-4-yl)-1- |
| | oxoisoindolin-2-yl)piperidine- |
| | 2,6-dione |
| I-184 | 3-(5-(3,3-dimethylpiperidin-4- |
| | yl)-1-oxoisoindolin-2- |
| | yl)piperidine-2,6-dione |
| I-185 | 3-(5-(1-benzyl-3,3- |
| | dimethylpiperidin-4-yl)-1- |
| | oxoisoindolin-2-yl)piperidine- |
| | 2,6-dione |
| I-186 | 5-(3-methylpiperidin-4-yl)-2-(2- |
| | oxopiperidin-3-yl)isoindolin-1- |
| | one |
| I-187 | 3-(5-(1-benzyl-3- |
| | methylpiperidin-4-yl)-1- |
| | oxoisoindolin-2-yl)piperidine- |
| | 2,6-dione |
| I-188 | 3-(5-(8-azabicyclo[3,2,1]octan- |
| | 3-yl)-1-oxoisoindolin-2- |
| | yl)piperidine-2,6-dione |
| I-189 | 3-(5-(1-(2-hydroxy-1- |
| | phenylethyl)piperidin-4-yl)-1- |
| | oxoisoindolin-2-yl)piperidine- |
| | 2,6-dione |
| I-190 | 3-(5-((S)-1-benzylazepan-4-yl)- |
| | 1-oxoisoindolin-2-yl)piperidine- |
| | 2,6-dione |
| I-191 | 3-(5-(1-benzyl-2,5-dihydro-1H- |
| | pyrrol-3-yl)-1-oxoisoindolin-2- |
| | yl)piperidine-2,6-dione |
| I-192 | 3-(5-(1-benzyl-2-oxo-1,2- |
| | dihydropyridin-4-yl)-1- |
| | oxoisoindolin-2-yl)piperidine- |
| | 2,6-dione |
| I-193 | 3-(5-(1-benzyl-2-oxopiperidin-4- |
| | yl)-1-oxoisoindolin-2- |
| | yl)piperidine-2,6-dione |
| I-194 | 3-(1-oxo-5-(2-oxopiperidin-4- |
| | yl)isoindolin-2-yl)piperidine- |
| | 2,6-dione |
| I-195 | 3-(1-oxo-5-(2-oxo-1,2- |
| | dihydropyridin-4-yl)isoindolin- |
| | 2-yl)piperidine-2,6-dione |
| I-196 | 3-(1-oxo-5-(1,2,3,4- |
| | tetrahydroquinolin-4- |
| | yl)isoindolin-2-yl)piperidine- |
| | 2,6-dione |
| I-197 | 3-(5-(1-benzyl-1,2,3,4- |
| | tetrahydroquinolin-4-yl)-1- |
| | oxoisoindolin-2-yl)piperidine- |
| | 2,6-dione |
| I-198 | 3-(5-(1-((1-benzyl-1H-tetrazol- |
| | 5-yl)methyl)piperidin-4-yl)-1- |
| | oxoisoindolin-2-yl)piperidine- |
| | 2,6-dione |
| I-199 | 3-(1-oxo-5-(1-((5-phenyl-1,3,4- |
| | oxadiazol-2-yl)methyl)piperidin- |
| | 4-yl)isoindolin-2-yl)piperidine- |
| | 2,6-dione |
| I-200 | 3-(5-(1-(benzo[d]thiazol-2- |
| | ylmethyl)piperidin-4-yl)-1- |
| | oxoisoindolin-2-yl)piperidine- |
| | 2,6-dione |
| I-201 | 3-(1-oxo-5-(1-((3-(pyridin-2-yl)- |
| | 1H-pyrazol-5- |
| | yl)methyl)piperidin-4- |
| | yl)isoindolin-2-yl)piperidine- |
| | 2,6-dione |
| I-202 | 3-(5-(1-((R)-2-hydroxy-1- |
| | phenylethyl)piperidin-4-yl)-1- |
| | oxoisoindolin-2-yl)piperidine- |
| | 2,6-dione |
| I-203 | 3-(5-(1-((1-methyl-1H-indazol- |
| | 3-yl)methyl)piperidin-4-yl)-1- |
| | oxoisoindolin-2-yl)piperidine- |
| | 2,6-dione |
| I-204 | 3-(5-(1-((1,2,4-oxadiazol-3- |
| | yl)methyl)piperidin-4-yl)-1- |
| | oxoisoindolin-2-yl)piperidine- |
| | 2,6-dione |
| I-205 | 3-(5-(1-(4-hydroxy-3-((4- |
| | methylpiperazin-1- |
| | yl)methyl)benzyl)piperidin-4- |
| | yl)-1-oxoisoindolin-2- |
| | yl)piperidine-2,6-dione |
| I-206 | 2-(4-((4-(2-(2,6-dioxopiperidin- |
| | 3-yl)-1-oxoisoindolin-5- |
| | yl)piperidin-1- |
| | yl)methyl)phenyl)acetonitrile |
| I-207 | 3-(5-(1-((2-(4-chlorophenyl)-5- |
| | methyloxazol-4- |
| | yl)methyl)piperidin-4-yl)-1- |
| | oxoisoindolin-2-yl)piperidine- |
| | 2,6-dione |
| I-208 | 3-(5-(1-((7-hydroxy-2- |
| | methylpyrazolo[1,5-a]pyrimidin- |
| | 5-yl)methyl)piperidin-4-yl)-1- |
| | oxoisoindolin-2-yl)piperidine- |
| | 2,6-dione |
| I-209 | 3-(5-(1-(2,2-difluoro-1- |
| | phenylethyl)piperidin-4-yl)-1- |
| | oxoisoindolin-2-yl)piperidine- |
| | 2,6-dione |
| I-210 | 3-(5-(1-((3- |
| | fluorobicyclo[1.1.1]pentan-1- |
| | yl)methyl)piperidin-4-yl)-1- |
| | oxoisoindolin-2-yl)piperidine- |
| | 2,6-dione |
| I-211 | 3-(1-oxo-5-(1-((2-phenylthiazol- |
| | 4-yl)methyl)piperidin-4- |
| | yl)isoindolin-2-yl)piperidine- |
| | 2,6-dione |
| I-212 | 3-(5-(1-(2-fluoro-1- |
| | phenylethyl)piperidin-4-yl)-1- |
| | oxoisoindolin-2-yl)piperidine- |
| | 2,6-dione |
| I-213 | 3-(1-oxo-5-(1-((4-oxo-3,4- |
| | dihydrothieno[3,2-d]pyrimidin- |
| | 2-yl)methyl)piperidin-4- |
| | yl)isoindolin-2-yl)piperidine- |
| | 2,6-dione |
| I-214 | 3-(1-oxo-5-(1-(quinolin-4- |
| | ylmethyl)piperidin-4- |
| | yl)isoindolin-2-yl)piperidine- |
| | 2,6-dione |
| I-215 | 3-(5-(1-(3,5- |
| | bis(trifluoromethyl)benzyl)piperidin- |
| | 4-yl)-1-oxoisoindolin-2- |
| | yl)piperidine-2,6-dione |
| I-216 | 3-((4-(2-(2,6-dioxopiperidin-3- |
| | yl)-1-oxoisoindolin-5- |
| | yl)piperidin-1-yl)methyl)-N,N- |
| | dimethylbenzenesulfonamide |
| I-217 | 6-((4-(2-(2,6-dioxopiperidin-3- |
| | yl)-1-oxoisoindolin-5- |
| | yl)piperidin-1- |
| | yl)methyl)picolinonitrile |
| I-218 | 2-(4-((4-(2-(2,6-dioxopiperidin- |
| | 3-yl)-1-oxoisoindolin-5- |
| | yl)piperidin-1- |
| | yl)methyl)phenoxy)acetonitrile |
| I-219 | 3-(5-(1-((1H-indazol-5- |
| | yl)methyl)piperidin-4-yl)-1- |
| | oxoisoindolin-2-yl)piperidine- |
| | 2,6-dione |
| I-220 | 3-(5-(1-(2,2- |
| | difluoroethyl)piperidin-4-yl)-1- |
| | oxoisoindolin-2-yl)piperidine- |
| | 2,6-dione |
| I-221 | 3-(5-(1-((7-methyl-4-oxo-4H- |
| | pyrido[1,2-a]pyrimidin-2- |
| | yl)methyl)piperidin-4-yl)-1- |
| | oxoisoindolin-2-yl)piperidine- |
| | 2,6-dione |
| I-222 | benzyl 4-(2-(2,6-dioxopiperidin- |
| | 3-yl)-1-oxoisoindolin-5- |
| | yl)piperidine-1-carboxylate |
| I-223 | 3-(1-oxo-5-(1-(2- |
| | phenylacetyl)piperidin-4- |
| | yl)isoindolin-2-yl)piperidine- |
| | 2,6-dione |
| I-224 | 3-(1-oxo-5-(1-(2,2,2-trifluoro-1- |
| | phenylethyl)piperidin-4- |
| | yl)isoindolin-2-yl)piperidine- |
| | 2,6-dione |
| I-225 | 3-(5-(1-(4-(5- |
| | methylbenzo[d]thiazol-2- |
| | yl)benzyl)piperidin-4-yl)-1- |
| | oxoisoindolin-2-yl)piperidine- |
| | 2,6-dione |
| I-226 | 3-(5-(1-(isoquinolin-1- |
| | ylmethyl)piperidin-4-yl)-1- |
| | oxoisoindolin-2-yl)piperidine- |
| | 2,6-dione |
| I-227 | 3-(5-(1-(4-(4-methoxypiperidin- |
| | 1-yl)benzyl)piperidin-4-yl)-1- |
| | oxoisoindolin-2-yl)piperidine- |
| | 2,6-dione |
| I-228 | 3-(5-(1-(4- |
| | (isopropylthio)benzyl)piperidin- |
| | 4-yl)-1-oxoisoindolin-2- |
| | yl)piperidine-2,6-dione |
| I-229 | tert-butyl (5-((4-(2-(2,6- |
| | dioxopiperidin-3-yl)-1- |
| | oxoisoindolin-5-yl)piperidin-1- |
| | yl)methyl)-4- |
| | (trifluoromethyl)thiazol-2- |
| | yl)carbamate |
| I-230 | 3-(1-oxo-5-(1-((S)-1- |
| | phenylethyl)piperidin-4- |
| | yl)isoindolin-2-yl)piperidine- |
| | 2,6-dione |
| I-231 | 2-(4-((4-(2-(2,6-dioxopiperidin- |
| | 3-yl)-1-oxoisoindolin-5- |
| | yl)piperidin-1- |
| | yl)methyl)phenyl)acetic acid |
| I-232 | 3-(5-(1-((7-fluoroquinolin-2- |
| | yl)methyl)piperidin-4-yl)-1- |
| | oxoisoindolin-2-yl)piperidine- |
| | 2,6-dione |
| I-233 | 3-(5-(1-((5-methyl-2-(4- |
| | (trifluoromethyl)phenyl)oxazol- |
| | 4-yl)methyl)piperidin-4-yl)-1- |
| | oxoisoindolin-2-yl)piperidine- |
| | 2,6-dione |
| I-234 | 3-(5-(1-((2-amino-4- |
| | (trifluoromethyl)thiazol-5- |
| | yl)methyl)piperidin-4-yl)-1- |
| | oxoisoindolin-2-yl)piperidine- |
| | 2,6-dione |
| I-235 | 3-((4-(2-(2,6-dioxopiperidin-3- |
| | yl)-1-oxoisoindolin-5- |
| | yl)piperidin-1-yl)methyl)-1,2,4- |
| | oxadiazole-5-carboxamide |
| I-236 | 3-(5-(1-(3- |
| | (morpholinosulfonyl)benzyl)piperidin- |
| | 4-yl)-1-oxoisoindolin-2- |
| | yl)piperidine-2,6-dione |
| I-237 | 4-((4-(2-(2,6-dioxopiperidin-3- |
| | yl)-1-oxoisoindolin-5- |
| | yl)piperidin-1-yl)methyl)-N.N- |
| | dimethylbenzenesulfonamide |
| I-238 | 3-(1-oxo-5-(1-(thiazol-4- |
| | ylmethyl)piperidin-4- |
| | yl)isoindolin-2-yl)piperidine- |
| | 2,6-dione |
| I-239 | 3-(1-oxo-5-(1-(quinoxalin-6- |
| | ylmethyl)piperidin-4- |
| | yl)isoindolin-2-yl)piperidine- |
| | 2,6-dione |
| I-240 | 3-(5-(1-((2-(4-fluorophenyl)-5- |
| | methyloxazol-4- |
| | yl)methyl)piperidin-4-yl)-1- |
| | oxoisoindolin-2-yl)piperidine- |
| | 2,6-dione |
| I-241 | 3-(1-oxo-5-(1-((3-(m-tolyl)- |
| | 1,2,4-oxadiazol-5- |
| | yl)methyl)piperidin-4- |
| | yl)isoindolin-2-yl)piperidine- |
| | 2,6-dione |
| I-242 | 3-(5-(1-(4-(tert- |
| | butyl)benzoyl)piperidin-4-yl)-1- |
| | oxoisoindolin-2-yl)piperidine- |
| | 2,6-dione |
| I-243 | 3-(1-oxo-5-(1-((5-(4- |
| | (trifluoromethyl)phenyl)-1,2,4- |
| | oxadiazol-3-yl)methyl)piperidin- |
| | 4-yl)isoindolin-2-yl)piperidine- |
| | 2,6-dione |
| I-244 | 3-(5-(1-(4-((4- |
| | fluorobenzyl)oxy)benzyl)piperidin- |
| | 4-yl)-1-oxoisoindolin-2- |
| | yl)piperidine-2,6-dione |
| I-245 | 3-(5-(1-((3-methylisoxazol-5- |
| | yl)methyl)piperidin-4-yl)-1- |
| | oxoisoindolin-2-yl)piperidine- |
| | 2,6-dione |
| I-246 | 3-(5-(1-(isoxazol-3- |
| | ylmethyl)piperidin-4-yl)-1- |
| | oxoisoindolin-2-yl)piperidine- |
| | 2,6-dione |
| I-247 | 3-(1-oxo-5-(1-((R)-1- |
| | phenylethyl)piperidin-4- |
| | yl)isoindolin-2-yl)piperidine- |
| | 2,6-dione |
| I-248 | 3-(5-(1-(4-(methoxymethyl)benz- |
| | yl)piperidin-4-yl)-1-oxoisoindolin-2- |
| | yl)piperidine-2,6-dione |
| I-249 | 3-(5-(1-((S)-2-hydroxy-1- |
| | phenylethyl)piperidin-4-yl)-1- |
| | oxoisoindolin-2-yl)piperidine- |
| | 2,6-dione |
| I-250 | 3-(1-oxo-5-(1- |
| | (phenylsulfonyl)piperidin-4- |
| | yl)isoindolin-2-yl)piperidine- |
| | 2,6-dione |
| I-251 | 3-(5-(1-((5-methyl-3- |
| | phenylisoxazol-4- |
| | yl)methyl)piperidin-4-yl)-1- |
| | oxoisoindolin-2-yl)piperidine- |
| | 2,6-dione |
| I-252 | 3-(5-(1-(4- |
| | ((difluoromethyl)sulfonyl)benz- |
| | yl)piperidin-4-yl)-1-oxoisoindolin- |
| | 2-yl)piperidine-2,6-dione |
| I-253 | 3-(1-oxo-5-(1-(2,2,2- |
| | trifluoroethyl)piperidin-4- |
| | yl)isoindolin-2-yl)piperidine- |
| | 2,6-dione |
| I-254 | methyl 2-((4-(2-(2,6- |
| | dioxopiperidin-3-yl)-1- |
| | oxoisoindolin-5-yl)piperidin-1- |
| | yl)methyl)oxazole-4-carboxylate |
| I-255 | 3-(1-oxo-5-(1-(4-(pyridin-2- |
| | ylmethoxy)benzyl)piperidin-4- |
| | yl)isoindolin-2-yl)piperidine- |
| | 2,6-dione |
| I-256 | 3-(5-(1-acetylpiperidin-4-yl)-1- |
| | oxoisoindolin-2-yl)piperidine- |
| | 2,6-dione |
| I-257 | 3-(5-(1-((5-methyl-2- |
| | phenyloxazol-4- |
| | yl)methyl)piperidin-4-yl)-1- |
| | oxoisoindolin-2-yl)piperidine- |
| | 2,6-dione |
| I-258 | 3-(5-(1-((3-cyclohexylisoxazol- |
| | 5-yl)methyl)piperidin-4-yl)-1- |
| | oxoisoindolin-2-yl)piperidine- |
| | 2,6-dione |
| I-259 | 3-(1-oxo-5-(1-((2-oxo-2,3- |
| | dihydro-1H-benzo[d]imidazol-5- |
| | yl)methyl)piperidin-4- |
| | yl)isoindolin-2-yl)piperidine- |
| | 2,6-dione |
| I-260 | 3-(5-(1-benzylpyrrolidin-3-yl)-1- |
| | oxoisoindolin-2-yl)piperidine- |
| | 2,6-dione |
| I-261 | (R)-3-(5-((R)-1-benzylazepan-4- |
| | yl)-1-oxoisoindolin-2-yl)piperidine- |
| | 2,6-dione |
| I-262 | (S)-3-(5-((S)-1-benzylazepan-4- |
| | yl)-1-oxoisoindolin-2-yl)piperidine- |
| | 2,6-dione |
| I-263 | 3-(5-(1-benzylazepan-4-yl)-1- |
| | oxoisoindolin-2-yl)piperidine- |
| | 2,6-dione |
| I-264 | 3-(5-(1-methyl-2,3,6,7- |
| | tetrahydro-1H-azepin-4-yl)-1- |
| | oxoisoindolin-2-yl)piperidine- |
| | 2,6-dione |
| I-265 | 3-(5-(8-benzyl-8- |
| | azabicyclo[3.2.1]octan-3-yl)-1- |
| | oxoisoindolin-2-yl)piperidine- |
| | 2,6-dione |
| I-266 | trans-3-(1-oxo-5-(1-((4- |
| | (trifluoromethyl)cyclohexyl)meth- |
| | yl)piperidin-4-yl)isoindolin-2- |
| | yl)piperidine-2,6-dione |
| I-267 | (S)-3-(1-oxo-5-((S)-piperidin-3- |
| | yl)isoindolin-2-yl)piperidine- |
| | 2,6-dione |
| I-268 | 3-(5-(1-acetyl-1,2,5,6- |
| | tetrahydropyridin-3-yl)-1- |
| | oxoisoindolin-2-yl)piperidine- |
| | 2,6-dione |
| I-269 | (R)-3-(5-((R)-1-acetylpyrrolidin- |
| | 3-yl)-1-oxoisoindolin-2- |
| | yl)piperidine-2,6-dione |
| I-270 | 3-(5-(1-acetyl-1,2,3,6- |
| | tetrahydropyridin-4-yl)-1- |
| | oxoisoindolin-2-yl)piperidine- |
| | 2,6-dione |
| I-271 | 3-(5-(octahydroindolizin-7-yl)-1- |
| | oxoisoindolin-2-yl)piperidine- |
| | 2,6-dione |
| I-272 | (R)-3-(5-((S)-1-benzylazepan-4- |
| | yl)-1-oxoisoindolin-2-yl)piperidine- |
| | 2,6-dione |
| I-273 | 3-(5-((R)-1-benzylazepan-4-yl)- |
| | 1-oxoisoindolin-2-yl)piperidine- |
| | 2,6-dione |
| I-274 | 3-(5-(2,5-dihydro-1H-pyrrol-3- |
| | yl)-1-oxoisoindolin-2- |
| | yl)piperidine-2,6-dione |
| I-275 | 3-(5-(1-acetyl-2,5-dihydro-1H- |
| | pyrrol-3-yl)-1-oxoisoindolin-2- |
| | yl)piperidine-2,6-dione |
| I-276 | cis-3-(1-oxo-5-(1-((4- |
| | (trifluoromethyl)cyclohexyl)meth- |
| | yl)piperidin-4-yl)isoindolin-2- |
| | yl)piperidine-2,6-dione |
| I-277 | 3-(1-oxo-5-(2,3,6,7-tetrahydro- |
| | 1H-azepin-4-yl)isoindolin-2- |
| | yl)piperidine-2,6-dione |
| I-278 | 3-(5-(1-methylazepan-4-yl)-1- |
| | oxoisoindolin-2-yl)piperidine- |
| | 2,6-dione |
| I-279 | (R)-3-(1-oxo-5-((S)-piperidin-3- |
| | yl)isoindolin-2-yl)piperidine- |
| | 2,6-dione |
| I-280 | 3-(1-oxo-5-(1,2,3,6- |
| | tetrahydropyridin-4- |
| | yl)isoindolin-2-yl)piperidine- |
| | 2,6-dione |
| I-281 | (S)-3-(5-((R)-1-benzylazepan-4- |
| | yl)-1-oxoisoindolin-2- |
| | yl)piperidine-2,6-dione |
| I-282 | 3-(1-oxo-5-(1,2,5,6- |
| | tetrahydropyridin-3- |
| | yl)isoindolin-2-yl)piperidine- |
| | 2,6-dione |
| I-283 | 3-(1-oxo-5-(2,2,6,6-tetramethyl- |
| | 1,2,3,6-tetrahydropyridin-4- |
| | yl)isoindolin-2-yl)piperidine- |
| | 2,6-dione |
| I-284 | (S)-3-(5-((R)-1-acetylpyrrolidin- |
| | 3-yl)-1-oxoisoindolin-2- |
| | yl)piperidine-2,6-dione |
| I-285 | 3-(5-(1-((6-isopropoxypyridin-3- |
| | yl)methyl)piperidin-4-yl)-1- |
| | oxoisoindolin-2-yl)piperidine- |
| | 2,6-dione |
| I-286 | 3-(1-oxo-5-(1-((1-phenyl-1H- |
| | pyrazol-5-yl)methyl)piperidin-4- |
| | yl)isoindolin-2-yl)piperidine- |
| | 2,6-dione |
| I-287 | 3-(5-(1-(4- |
| | ethoxybenzyl)piperidin-4-yl)-1- |
| | oxoisoindolin-2-yl)piperidine- |
| | 2,6-dione |
| I-288 | 3-(1-oxo-5-(1-((1-phenyl-1H- |
| | pyrazol-4-yl)methyl)piperidin-4- |
| | yl)isoindolin-2-yl)piperidine- |
| | 2,6-dione |
| I-289 | 3-(5-(1-((1-isopropyl-1H- |
| | pyrazol-5-yl)methyl)piperidin-4- |
| | yl)-1-oxoisoindolin-2- |
| | yl)piperidine-2,6-dione |
| I-290 | 3-(5-(1-(isothiazol-5- |
| | ylmethyl)piperidin-4-yl)-1- |
| | oxoisoindolin-2-yl)piperidine- |
| | 2,6-dione |
| I-291 | 3-(5-(1-((1-isopropyl-1H- |
| | pyrazol-4-yl)methyl)piperidin-4- |
| | yl)-1-oxoisoindolin-2- |
| | yl)piperidine-2,6-dione |
| I-292 | 3-(5-(1-((1H-pyrazol-5- |
| | yl)methyl)piperidin-4-yl)-1- |
| | oxoisoindolin-2-yl)piperidine- |
| | 2,6-dione |
| I-293 | 3-(5-(1-((5-isopropoxypyridin-2- |
| | yl)methyl)piperidin-4-yl)-1- |
| | oxoisoindolin-2-yl)piperidine- |
| | 2,6-dione |
| I-294 | 3-(1-oxo-5-(1-((1-(pyridin-3-yl)- |
| | 1H-pyrazol-5- |
| | yl)methyl)piperidin-4- |
| | yl)isoindolin-2-yl)piperidine- |
| | 2,6-dione |
| I-295 | 3-(1-oxo-5-(1-((1-(pyridin-3-yl)- |
| | 1H-pyrazol-4- |
| | yl)methyl)piperidin-4- |
| | yl)isoindolin-2-yl)piperidine- |
| | 2,6-dione |
| I-296 | 5-((4-(2-(2,6-dioxopiperidin-3- |
| | yl)-1-oxoisoindolin-5- |
| | yl)piperidin-1-yl)methyl)-2- |
| | fluorobenzonitrile |
| I-297 | 3-(5-(1-((5-fluoropyridin-2- |
| | yl)methyl)piperidin-4-yl)-1- |
| | oxoisoindolin-2-yl)piperidine- |
| | 2,6-dione |
| I-298 | 3-(5-(1-((1-ethyl-3-(pyridin-3- |
| | yl)-1H-pyrazol-4- |
| | yl)methyl)piperidin-4-yl)-1- |
| | oxoisoindolin-2-yl)piperidine- |
| | 2,6-dione |
| I-299 | 3-(5-(1-((6-methoxypyridin-2- |
| | yl)methyl)piperidin-4-yl)-1- |
| | oxoisoindolin-2-yl)piperidine- |
| | 2,6-dione |
| I-300 | 3-(5-(1-((3-((3S,5S)-adamantan- |
| | 1-yl)-1H-pyrazol-5- |
| | yl)methyl)piperidin-4-yl)-1- |
| | oxoisoindolin-2-yl)piperidine- |
| | 2,6-dione |
| I-301 | 3-(5-(1-((6-isopropoxypyridin-2- |
| | yl)methyl)piperidin-4-yl)-1- |
| | oxoisoindolin-2-yl)piperidine- |
| | 2,6-dione |
| I-302 | 3-(5-(1-((1-benzyl-5-(pyridin-2- |
| | yl)-1H-pyrazol-3- |
| | yl)methyl)piperidin-4-yl)-1- |
| | oxoisoindolin-2-yl)piperidine- |
| | 2,6-dione |
| I-303 | trans-3-(5-(1-((4- |
| | methoxycyclohexyl)methyl)pipe |
| | ridin-4-yl)-1-oxoisoindolin-2- |
| | yl)piperidine-2,6-dione |

### Degradation of CCAR Mediated by Degradation Domains and Stabilization Compounds

In some embodiments, provided herein is a fusion polypeptide, e.g., CCAR, comprising a degradation domain and a heterologous polypeptide, e.g., CAR. In some embodiments, the degradation domain has a first state and a second state, e.g., states of stabilization/destabilization, or states of folding/misfolding. The first state is associated with, causes, or mediates expression of the fusion polypeptide, e.g., CCAR, at a first rate or level and the second state is associated with, causes, or mediates expression of the fusion polypeptide, e.g., CCAR, at a second rate or level. In some embodiments, the second state has a level or rate that is greater, e.g., 2, 3, 4, 5, 6, 7, 8, 9, 10, 20, or 30 fold greater, than the rate or level of the first state. In some embodiments, the second state is associated with, maintained by, or caused by the presence of a stabilization compound. In some embodiments, the presence of the stabilization compound can be associated with, cause, or mediate the transformation of a first folding state to a second folding state, e.g., from misfolded to more properly folded state, e.g., a first state susceptible to degradation to a second state less susceptible to degradation than the first state; or from a first folding state that has a first level of degradation to a second folding state what has a second, lessor, level of degradation, e.g., in a cell of interest.

Without wishing to be bound by theory, in some embodiments, the degradation domain is unstable and/or unable to fold into a stable conformation in the absence of a stabilization compound. This misfolded/unfolded degradation domain can be degraded by intracellular degradation pathway along with the rest of the fusion polypeptide, e.g., CCAR. In the presence of the stabilization compound, the degradation domain assumes a proper conformation and is less susceptible to intercellular degradation pathways. Thus, the expression level of the fusion polypeptide, e.g., CCAR, can be regulated by the presence or absence of the stabilization compound. In some embodiments, the expression level of the fusion polypeptide, e.g., CCAR, in the presence of the stabilization compound is increased by at least, e.g., 1.5-, 2-, 3-, 4-, 5-, 10-, 20-, 30-, 40-, or 50-fold, compared to the expression level of the fusion polypeptide, e.g., CCAR, in the absence of the stabilization compound, e.g., as measured by an assay described herein, e.g., a Western blot analysis or a flow cytometry analysis.

In some embodiments, the degradation domain is separated from the heterologous polypeptide, e.g., CAR, by a heterologous protease cleavage site. In some embodiments, the proper folding of the degradation domain exposes the heterologous protease cleavage site, leading to the cleavage of the heterologous protease cleavage site and the removal of the degradation domain from the rest of the fusion polypeptide, e.g., CCAR.

### Degradation Domains and Stabilization Compounds

The present disclosure encompasses degradation domains derived from any naturally occurring protein. Preferably, fusion polypeptides, e.g., CCARs, of this disclosure will include a degradation domain for which there is no ligand natively expressed in the cell compartments of interest. For example, if the fusion polypeptide, e.g., CCAR, is designed for expression in T cells, it is preferable to select a degradation domain for which there is no naturally occurring ligand present in T cells. Thus, the degradation domain, when expressed in the cell of interest, will only be stabilized in the presence of an exogenously added compound. Notably, this property can be engineered by either engineering the degradation domain to no longer bind a natively expressed ligand (in which case the degradation domain will only be stable in the presence of a synthetic compound) or by expressing the degradation domain in a compartment where the natively expressed ligand does not occur (e.g., the degradation domain can be derived from a species other than the species in which the fusion polypeptide, e.g., CCAR, will be expressed).

Degradation domain—stabilization compound pairs can be derived from any naturally occurring or synthetically developed protein. Stabilization compounds can be any naturally occurring or synthetic compounds. In certain embodiments, the stabilization compounds will be existing prescription or over-the-counter medicines. Examples of proteins that can be engineered to possess the properties of a degradation domain are set forth in Table 32 below along with a corresponding stabilization compound.

In some embodiments, the degradation domain is based on FKBP (e.g., using a “Shield” stabilization compound) as described in: Banaszynski, et al., Cell, 2006, 126, 995-1004; based on DHFR (e.g., using trimethoprim as a stabilization compound) as described in Iwamoto, et al., Chemistry & Biology, 2010, 17, 981-988; or based on estrogen receptor alpha (e.g., where 4OHT is used as a stabilization compound) as described in Miyazaki, et al., J. Am. Chem. Soc. 2012, 134, 3942-3945. Each of these references is incorporated by reference in its entirety.

In some embodiments, the degradation domain is derived from a protein listed in Table 32.

In some embodiments, the degradation domain is derived from an estrogen receptor (ER). In some embodiments, the degradation domain comprises an amino acid sequence selected from SEQ ID NO: 342 or a sequence having at least 90%, 95%, 97%, 98%, or 99% identity thereto, or SEQ ID NO: 344 or a sequence having at least 90%, 95%, 97%, 98%, or 99% identity thereto. In some embodiments, the degradation domain comprises the amino acid sequence of SEQ ID NO: 342 or 344. When the degradation domain is derived from an estrogen receptor, the stabilization compound can be selected from Bazedoxifene or 4-hydroxy tamoxifen (4-OHT). In some embodiments, the stabilization compound is Bazedoxifene. Tamoxifen and Bazedoxifene are FDA approved drugs, and thus are safe to use in human.

In some embodiments, the degradation domain is derived from an FKB protein (FKBP). In some embodiments, the degradation domain comprises the amino acid sequence of SEQ ID NO: 346 or a sequence having at least 90%, 95%, 97%, 98%, or 99% identity thereto. In some embodiments, the degradation domain comprises the amino acid sequence of SEQ ID NO: 346. When the degradation domain is derived from a FKBP, the stabilization compound can be Shield-1.

In some embodiments, the degradation domain is derived from dihydrofolate reductase (DHFR). In some embodiments, the degradation domain comprises the amino acid sequence of SEQ ID NO: 347 or a sequence having at least 90%, 95%, 97%, 98%, or 99% identity thereto. In some embodiments, the degradation domain comprises the amino acid sequence of SEQ ID NO: 347. When the degradation domain is derived from a DHFR, the stabilization compound can be Trimethoprim.

In some embodiments, the degradation domain is not derived from an FKB protein, estrogen receptor, or DHFR.

TABLE 32
Exemplary proteins for generating degradation domains

| | | |
|---|---|---|
| Type | Activity of drug | Drug examples |

Oxidoreductases

| | | |
|---|---|---|
| Aldehyde dehydrogenase | Inhibitor | Disulfiram |
| Monoamine oxidases (MAOs) | MAO-A inhibitor | Tranylcypromine, moclobemide |
| | MAO-B inhibitor | Tranylcypromine |
| Cyclooxygenases (COXs) | COX1 inhibitor | Acetylsalicylic acid, profens, |
| | | acetaminophen and dipyrone (as |
| | | arachidonylamides) |
| | COX2 inhibitor | Acetylsalicylic acid, profens, |
| | | acetaminophen and dipyrone (as |
| | | arachidonylamides) |
| Vitamin K epoxide reductase | Inhibitor | Warfarin, phenprocoumon |
| Aromatase | Inhibitor | Exemestane |
| Lanosterol demethylase | Inhibitor | Azole antifungals |
| (fungal) | | |
| Lipoxygenases | Inhibitor | Mesalazine |
| | 5-lipoxygenase inhibitor | Zileuton |
| Thyroidal peroxidase | Inhibitor | Thiouracils |
| Iodothyronine-5′ deiodinase | Inhibitor | Propylthiouracil |
| Inosine monophosphate | Inhibitor | Mycophenolate mofetil |
| dehydrogenase | | |
| HMG-CoA reductase | Inhibitor | Statins |
| α-5-Testosterone reductase | Inhibitor | Finasteride, dutasteride |
| Dihydrofolate reductase | Inhibitor | Trimethoprim |
| (bacterial) | | |
| Dihydrofolate reductase | Inhibitor | Methotrexate, pemetrexed |
| (human) | | |
| Dihydrofolate reductase | Inhibitor | Proguanil |
| (parasitic) | | |
| Dihydroorotate reductase | Inhibitor | Leflunomide |
| Enoyl reductase | Inhibitor | Isoniazid |
| (mycobacterial) | | |
| Squalene epoxidase (fungal) | Inhibitor | Terbinafin |
| Δ-14 reductase (fungal) | Inhibitor | Amorolfin |
| Xanthine oxidase | Inhibitor | Allopurinol |
| 4-Hydroxyphenylpyruvate | Inhibitor | Nitisinone |
| dioxygenase | | |
| Ribonucleoside diphosphate | Inhibitor | Hydroxycarbamide |
| reductase | | |

Transferases

| | | |
|---|---|---|
| Protein kinase C | Inhibitor | Miltefosine |
| Bacterial peptidyl transferase | Inhibitor | Chloramphenicol |
| Catecholamine-O- | Inhibitor | Entacapone |
| methyltransferase | | |
| RNA polymerase (bacterial) | Inhibitor | Ansamycins |
| Reverse transcriptases (viral) | Competitive inhibitors | Zidovudine |
| | Allosteric inhibitors | Efavirenz |
| DNA polymerases | Inhibitor | Acyclovir, suramin |
| GABA transaminase | Inhibitor | Valproic acid, vigabatrin |
| Tyrosine kinases | PDGFR/ABL/KIT inhibitor | Imatinib |
| | EGFR inhibitor | Erlotinib |
| | β-VEGFR2/PDGFR/KIT/FLT3 | Sunitinib |
| | β-VEGFR2/PDGFR/RAF | Sorafenib |
| Glycinamide ribonucleotide | Inhibitor | Pemetrexed |
| formyl transferase | | |
| Phosphoenolpyruvate | Inhibitor | Fosfomycin |
| transferase (MurA, bacterial) | | |
| Human cytosolic branched- | Inhibitor | Gabapentin |
| chain aminotransferase | | |
| (hBCATc) | | |

Hydrolases (proteases)

| | | |
|---|---|---|
| Aspartyl proteases (viral) | HIV protease inhibitor | Saquinavir, indinavir |

Hydrolases (serine proteases)

| | | |
|---|---|---|
| Unspecific | Unspecific inhibitors | Aprotinine |
| Bacterial serine protease | Direct inhibitor | β-lactams |
| Bacterial serine protease | Indirect inhibitor | Glycopeptides |
| Bacterial lactamases | Direct inhibitor | Sulbactam |
| Human antithrombin | Activator | Heparins |
| Human plasminogen | Activator | Streptokinase |
| Human coagulation factor | Activator | Factor IX complex, Factor VIII |
| Human factor Xa | Inhibitor | Fondaparinux |

Hydrolases (metalloproteases)

| | | |
|---|---|---|
| Human ACE | Inhibitor | Captopril |
| Human HRD | Inhibitor | Cilastatin |
| Human carboxypeptidase A | Inhibitor | Penicillamine |
| (Zn) | | |
| Human enkephalinase | Inhibitor | Racecadotril |

Hydrolases (other)

| | | |
|---|---|---|
| 26S proteasome | Inhibitor | Bortezomib |
| Esterases | AChE inhibitor | Physostigmine |
| | AChE reactivators | Obidoxime |
| | PDE inhibitor | Caffeine |
| | PDE3 inhibitor | Amrinon, milrinone |
| | PDE4 inhibitor | Papaverine |
| | PDE5 inhibitor | Sildenafil |
| | HDAC inhibitor | Valproic acid |
| | HDAC3/HDAC7 inhibitor | Carbamezepine |
| Glycosidases (viral) | α-glycosidase inhibitor | Zanamivir, oseltamivir |
| Glycosidases (human) | α-glycosidase inhibitor | Acarbose |
| Lipases | Gastrointestinal lipases inhibitor | Orlistat |
| Phosphatases | Calcineurin inhibitor | Cyclosporin |
| | Inositol polyphosphate | Lithium ions |
| | phosphatase inhibitor | |
| GTPases | Rac1 inhibitor | 6-Thio-GTP (azathioprine |
| | | metabolite) |
| Phosphorylases | Bacterial C55-lipid phosphate | Bacitracin |
| | dephosphorylase inhibitor | |

Lyases

| | | |
|---|---|---|
| DOPA decarboxylase | Inhibitor | Carbidopa |
| Carbonic anhydrase | Inhibitor | Acetazolamide |
| Histidine decarboxylase | Inhibitor | Tritoqualine |
| Ornithine decarboxylase | Inhibitor | Eflornithine |
| Soluble guanylyl cyclase | Activator | Nitric acid esters, molsidomine |

Isomerases

| | | |
|---|---|---|
| Alanine racemase | Inhibitor | D-Cycloserine |
| DNA gyrases (bacterial) | Inhibitor | Fluoroquinolones |
| Topoisomerases | Topoisomerase I inhibitor | Irinotecan |
| | Topoisomerase II inhibitor | Etoposide |
| 8,7 isomerase (fungal) | Inhibitor | Amorolfin |

Ligases (also known as synthases)

| | | |
|---|---|---|
| Dihydropteroate synthase | Inhibitor | Sulphonamides |
| Thymidylate synthase (fungal | Inhibitor | Fluorouracil |
| and human) | | |
| Thymidylate synthase (human) | Inhibitor | Methotrexate, pemetrexed |
| Phosphofructokinase | Inhibitor | Antimony compounds |
| mTOR | Inhibitor | Rapamycin |
| Haem polymerase | Inhibitor | Quinoline antimalarials |
| (Plasmodium) | | |
| β-1,3--D-glucansynthase | Inhibitor | Caspofungin |
| (fungi) | | |
| Glucosylceramide synthase | Inhibitor | Miglustat |

| Substrate | Drug substance |
|---|---|
| Asparagine | Asparaginase |
| Urate | Rasburicase (a urate oxidase) |
| VAMP-synaptobrevin, | Light chain of the botulinum |
| SNAP25, Syntaxin | neurotoxin (Zn-endopeptidase) |

| | | |
|---|---|---|
| Type | Activity of drug | Drug examples |

Direct ligand-gated ion channel receptors

| | | |
|---|---|---|
| GABA_{A} receptors | Barbiturate binding site agonists | Barbiturate |
| | Benzodiazepine binding site | Benzodiazepines |
| | agonists | |
| | Benzodiazepine binding site | Flumazenil |
| | antagonists | |
| Acetylcholine receptors | Nicotinic receptor agonists | Pyrantel (of Angiostrongylus), |
| | | levamisole |
| | Nicotinic receptor stabilizing | Alcuronium |
| | antagonists | |
| | Nicotinic receptor depolarizing | Suxamethonium |
| | antagonists | |
| | Nicotinic receptor allosteric | Galantamine |
| | modulators | |
| Glutamate receptors | NMDA subtype antagonists | Memantine |
| (ionotropic) | | |
| | NMDA subtype expression | Acamprosate |
| | modulators | |
| | NMDA subtype phencyclidine | Ketamine |
| | binding site antagonists | |

G-protein-coupled receptors

| | | |
|---|---|---|
| Acetylcholine receptors | Muscarinic receptor agonists | Pilocarpine |
| | Muscarinic receptor antagonists | Tropane derivatives |
| | Muscarinic receptor | Darifenacine |
| | M₃ antagonists | |
| Adenosine receptors | Agonists | Adenosine |
| | Adenosine A₁ receptor agonists | Lignans from valerian |
| | Adenosine A1 receptor | Caffeine, theophylline |
| | antagonists | |
| | Adenosine A_{2A} receptor | Caffeine, theophylline |
| | antagonists | |
| Adrenoceptors | Agonists | Adrenaline, noradrenaline, |
| | | ephedrine |
| | α₁- and α₂-receptors agonists | Xylometazoline |
| | α₁-receptor antagonists | Ergotamine |
| | α₂-receptor, central agonists | Methyldopa (as |
| | | methylnoradrenaline) |
| | β-adrenoceptor antagonists | Isoprenaline |
| | β₁-receptor antagonists | Propranolol, atenolol |
| | β₂-receptor agonists | Salbutamol |
| | β₂-receptor antagonists | Propranolol |
| Angiotensin receptors | AT₁-receptors antagonists | Sartans |
| Calcium-sensing receptor | Agonists | Strontium ions |
| | Allosteric activators | Cinacalcet |
| Cannabinoid receptors | CB₁ - and CB₂-receptors | Dronabinol |
| | agonists | |
| Cysteinyl-leukotriene receptors | Antagonists | Montelukast |
| Dopamine receptors | Dopamine receptor subtype | Dopamine, levodopa |
| | direct agonists | |
| | D₂, D₃ and D₄ agonists | Apomorphine |
| | D₂, D₃ and D₄ antagonists | Chlorpromazine, fluphenazine, |
| | | haloperidol, metoclopramide, |
| | | ziprasidone |
| Endothelin receptors (ET_{A}, ET_{B}) | Antagonists | Bosentan |
| GABA_{B} receptors | Agonists | Baclofen |
| Glucagon receptors | Agonists | Glucagon |
| Glucagon-like peptide-1 | Agonists | Exenatide |
| receptor | | |
| Histamine receptors | H₁-antagonists | Diphenhydramine |
| | H₂-antagonists | Cimetidine |
| Opioid receptors | μ-opioid agonists | Morphine, buprenorphine |
| | μ-, κ- and δ-opioid antagonists | Naltrexone |
| | κ-opioid antagonists | Buprenorphine |
| Neurokinin receptors | NK₁ receptor antagonists | Aprepitant |
| Prostanoid receptors | Agonists | Misoprostol, sulprostone, iloprost |
| Prostamide receptors | Agonists | Bimatoprost |
| Purinergic receptors | P₂Y₁₂ antagonists | Clopidogrel |
| Serotonin receptors | Subtype-specific (partial) | Ergometrine, ergotamine |
| | agonists | |
| | 5-HT_{1A} partial agonists | Buspirone |
| | 5-HT_{1B/1D} agonists | Triptans |
| | 5-HT_{2A} antagonists | Quetiapine, ziprasidone |
| | 5-HT₃ antagonists | Granisetron |
| | 5-HT₄ partial agonists | Tegaserode |
| Vasopressin receptors | Agonists | Vasopressin |
| | V₁ agonists | Terlipressin |
| | V₂ agonists | Desmopressin |
| | OT agonists | Oxytocin |
| | OT antagonists | Atosiban |

Cytokine receptors

| | | |
|---|---|---|
| Class I cytokine receptors | Growth hormone receptor | Pegvisomant |
| | antagonists | |
| | Erythropoietin receptor | Erythropoietin |
| | agonists | |
| | Granulocyte colony stimulating | Filgrastim |
| | factor agonists | |
| | Granulocyte-macrophage | Molgramostim |
| | colony stimulating factor | |
| | agonists | |
| | Interleukin-1 receptor | Anakinra |
| | antagonists | |
| | Interleukin-2 receptor agonists | Aldesleukin |
| TNFα receptors | Mimetics (soluble) | Etanercept |

Integrin receptors

| | | |
|---|---|---|
| Glycoprotein IIb/IIIa receptor | Antagonists | Tirofiban |

Receptors associated with a tyrosine kinase

| | | |
|---|---|---|
| Insulin receptor | Direct agonists | Insulin |
| Insulin receptor | Sensitizers | Biguanides |

Nuclear receptors (steroid hormone receptors)

| | | |
|---|---|---|
| Mineralocorticoid receptor | Agonists | Aldosterone |
| | Antagonists | Spironolactone |
| Glucocorticoid receptor | Agonists | Glucocorticoids |
| Progesterone receptor | Agonists | Gestagens |
| Estrogen receptor | Agonists | Oestrogens |
| | (Partial) antagonists | Clomifene |
| | Antagonists | Fulvestrant |
| | Modulators | Tamoxifen, raloxifene |
| | Androgen receptor Agonists | Testosterone |
| | Antagonists | Cyproterone acetate |
| Vitamin D receptor | Agonists | Retinoids |
| ACTH receptor agonists | Agonists | Tetracosactide (also known as |
| | | cosyntropin) |

Nuclear receptors (other)

| | | |
|---|---|---|
| | α-Retinoic acid receptors | Isotretinoin |
| | RAR agonists | |
| | β-RAR agonists | Adapalene, isotretinoin |
| | γ-RAR agonists | Adapalene, isotretinoin |
| Peroxisome proliferator- | α-PPAR agonists | Fibrates |
| activated receptor (PPAR) | | |
| | γ-PPAR agonists | Glitazones |
| Thyroid hormone receptors | Agonists | L-Thyroxine |

Voltage-gated Ca²⁺ channels

| | | |
|---|---|---|
| General | Inhibitor | Oxcarbazepine |
| In Schistosoma sp. | Inhibitor | Praziquantel |
| L-type channels | Inhibitor | Dihydropyridines, diltiazem, |
| | | lercanidipine, pregabalin, |
| | | verapamil |
| T-type channels | Inhibitor | Succinimides |

K+ channels

| | | |
|---|---|---|
| Epithelial K⁺ channels | Opener Inhibitor | Diazoxide, minoxidil |
| | | Nateglinide, sulphonylureas |
| Voltage-gated K⁺ channels | Inhibitor | Amiodarone |

Na⁺ channels

| | | |
|---|---|---|
| Epithelial Na+ channels (ENaC) | Inhibitor | Amiloride, bupivacaine, |
| | | lidocaine, procainamide, |
| | | quinidine |
| Voltage-gated Na⁺ channels | Inhibitor | Carbamazepine, flecainide, |
| | | lamotrigine, phenytoin, |
| | | propafenone, topiramate, |
| | | valproic acid |

Ryanodine-inositol 1,4,5-triphosphate receptor Ca²⁺ channel (RIR-CaC) family

| | | |
|---|---|---|
| Ryanodine receptors | Inhibitor | Dantrolene |

Transient receptor potential Ca²⁺ channel (TRP-CC) family

| | | |
|---|---|---|
| TRPV1 receptors | Inhibitor | Acetaminophen (as |
| | | arachidonylamide) |

Cl− channels

| | | |
|---|---|---|
| Cl⁻channel | Inhibitor (mast cells) Opener | Cromolyn sodium Ivermectin |
| | (parasites) | |
| Cation-chloride cotransporter | Thiazide-sensitive NaCl | Thiazide diuretics |
| (CCC) family | symporter, human inhibitor | |
| | Bumetanide-sensitive | Furosemide |
| | NaCl/KCl symporters, human | |
| | inhibitor | |
| Na⁺/H⁺ antiporters | Inhibitor | Amiloride, triamterene |
| Proton pumps | Ca²⁺-dependent ATPase | Artemisinin and derivatives |
| | (PfATP6; Plasmodia) inhibitor | |
| H⁺/K⁺-ATPase | Inhibitor | Omeprazole |
| Na⁺/K⁺ ATPase | Inhibitor | Cardiac glycosides |
| Eukaryotic (putative) sterol | Niemann-Pick C1 like 1 | Ezetimibe |
| transporter (EST) family | (NPC1L1) protein inhibitor | |
| Neurotransmitter/Na⁺ symporter | Serotonin/Na⁺ symporter | Cocaine, tricyclic |
| (NSS) family | inhibitor | antidepressants, paroxetine |
| | Noradrenaline/Na⁺ symporter | Bupropion, venlafaxine |
| | inhibitor | |
| | Dopamine/Na⁺ symporter | Tricyclic antidepressants, |
| | inhibitor | cocaine, amphetamines |
| | Vesicular monoamine | Reserpine |
| | transporter inhibitor | |

Nucleic acids

| | | |
|---|---|---|
| DNA and RNA | Alkylation | Chlorambucil, |
| | | cyclophosphamide, dacarbazine |
| | Complexation | Cisplatin |
| | Intercalation | Doxorubicin |
| | Oxidative degradation | Bleomycin |
| | Strand breaks | Nitroimidazoles |
| RNA | Interaction with 16S-rRNA | Aminoglycoside antiinfectives |
| | Interaction with 23S-rRNA | Macrolide antiinfectives |
| | 23S-rRNA/tRNA/2-polypeptide | Oxazolidinone antiinfectives |
| | complex | |
| Spindle | Inhibition of development | Vinca alkaloids |
| | Inhibition of desaggregation | Taxanes |
| Inhibition of mitosis | — | Colchicine |

Ribosome

| | | |
|---|---|---|
| 30S subunit (bacterial) | Inhibitors | Tetracyclines |
| 50S subunit (bacterial) | Inhibitors | Lincosamides, quinupristin- |
| | | dalfopristin |

TABLE 27
Exemplary sequences of a degradation domain

| SEQ | De- | |
|---|---|---|
| ID | scrip- | |
| NO | tion | Sequence |
| SEQ | ER1 WT | SLALSLTADQMVSALLDAEPPILYSEYDPTRPFSE |
| ID | (305aa- | ASMMGLLTNLADRELVHMINWAKRVPGFVDLTLHD |
| NO: | 549aa) | QVHLLECAWLEILMIGLVWRSMEHPGKLLFAPNLL |
| 340 | amino | LDRNQGKCVEGMVEIFDMLLATSSRFRMMNLQGEE |
| | acid | FVCLKSIILLNSGVYTFLSSTLKSLEEKDHIHRVL |
| | se- | DKITDTLIHLMAKAGLTLQQQHQRLAQLLLILSHI |
| | quence | RHMSNKGMEHLYSMKCKNVVPLYDLLLEMLDAHRL |
| SEQ | ER1 WT | tcgttggcactttccctgactgccgaccagatggt |
| ID | (305aa- | gtccgcccttctggacgccgagcctccaattctgt |
| NO: | 549aa) | actcggagtacgatccgactcgcccgttctccgaa |
| 341 | nucleo- | gccagcatgatgggcctgttgactaacctggcgga |
| | tide | ccgcgagttggtgcacatgattaactgggctaagc |
| | se- | gggtgccgggcttcgtggacctgactctgcacgac |
| | quence | caagtgcacctcctggaatgcgcctggctggaaat |
| | | cctcatgatcggcctcgtgtggagatccatggagc |
| | | atcccggaaagctcctgtttgcacccaacctcctg |
| | | cttgatcgcaaccagggaaaatgcgtggaagggat |
| | | ggtcgagattttcgacatgctgctcgccacctctt |
| | | cccggttccggatgatgaatctgcagggagaagag |
| | | ttcgtgtgtctgaagtcaatcatcctgctgaactc |
| | | cggggtctataccttcctgagctcgaccctcaagt |
| | | cactggaggaaaaagaccacatccatcgcgtgctc |
| | | gataagatcaccgacacccttatccatctcatggc |
| | | gaaggctggactgaccctgcaacagcagcaccaga |
| | | ggctggcccagttgctgctgattctgagccacatc |
| | | cggcacatgtcgaacaaggggatggaacacctgta |
| | | cagcatgaagtgcaagaacgtcgtgcctctgtacg |
| | | atctgctcctggaaatgctggacgcgcacagactc |
| SEQ | ERmut1 | SLALSLTADQMVSALLDAEPPILYSEYDPTRPFSE |
| ID | (6 | ASMMGLLTNLADRELVHMINWAKRVPGFVDLALHD |
| NO: | muta- | QVHLLECAWMEILMIGLVWRSMEHPGKLLFAPNLL |
| 342 | tions) | LDRNQGKCVEGGVEIFDMLLATSSRFRMMNLQGEE |
| | amino | FVCLKSIILLNSGVYTFLSSTLKSLEEKDHIHRVL |
| | acid | DKITDTLIHLMAKAGLTLQQQHQRLAQLLLILSHI |
| | se- | RHMSSKRMEHLYSMKCKNVVPLSDLLLEMLDAHRL |
| | quence | |
| SEQ | ERmut1 | tcgttggcactttccctgactgccgaccagatggt |
| ID | (6 | gtccgcccttctggacgccgagcctccaattctgt |
| NO: | muta- | actcggagtacgatccgactcgcccgttctccgaa |
| 343 | tions) | gccagcatgatgggcctgttgactaacctggcgga |
| | nucleo- | ccgcgagttggtgcacatgattaactgggctaagc |
| | tide | gggtgccgggcttcgtggacctggccctgcacgac |
| | se- | caagtgcacctcctggaatgcgcctggatggaaat |
| | quence | cctcatgatcggcctcgtgtggagatccatggagc |
| | | atcccggaaagctcctgtttgcacccaacctcctg |
| | | cttgatcgcaaccagggaaaatgcgtggaaggggg |
| | | tgtcgagattttcgacatgctgctcgccacctctt |
| | | cccggttccggatgatgaatctgcagggagaagag |
| | | ttcgtgtgtctgaagtcaatcatcctgctgaactc |
| | | cggggtctataccttcctgagctcgaccctcaagt |
| | | cactggaggaaaaagaccacatccatcgcgtgctc |
| | | gataagatcaccgacacccttatccatctcatggc |
| | | gaaggctggactgaccctgcaacagcagcaccaga |
| | | ggctggcccagttgctgctgattctgagccacatc |
| | | cggcacatgtcgtccaagaggatggaacacctgta |
| | | cagcatgaagtgcaagaacgtcgtgcctctgtccg |
| | | atctgctcctggaaatgctggacgcgcacagactc |
| SEQ | ERmut2 | SLALSLTADQMVSALLDAEPPILYSEYDPTRPFSE |
| ID | (4 | ASMMGLLTNLADRELVHMINWAKRVPGFVDLTLHD |
| NO: | muta- | QVHLLECAWMEILMIGLVWRSMEHPGKLLFAPNLL |
| 344 | tions) | LDRNQGKCVEGGVEIFDMLLATSSRFRMMNLQGEE |
| | amino | FVCLKSIILLNSGVYTFLSSTLKSLEEKDHIHRVL |
| | acid | DKITDTLIHLMAKAGLTLQQQHQRLAQLLLILSHI |
| | se- | RHMSNKRMEHLYSMKCKNVVPLSDLLLEMLDAHRL |
| | quence | |
| SEQ | ERmut2 | tcgttggcactttccctgactgccgaccagatggt |
| ID | (4 | gtccgcccttctggacgccgagcctccaattctgt |
| NO: | muta- | actcggagtacgatccgactcgcccgttctccgaa |
| 345 | tions) | gccagcatgatgggcctgttgactaacctggcgga |
| | nucleo- | ccgcgagttggtgcacatgattaactgggctaagc |
| | tide | gggtgccgggcttcgtggacctgaccctgcacgac |
| | se- | caagtgcacctcctggaatgcgcctggatggaaat |
| | quence | cctcatgatcggcctcgtgtggagatccatggagc |
| | | atcccggaaagctcctgtttgcacccaacctcctg |
| | | cttgatcgcaaccagggaaaatgcgtggaaggggg |
| | | tgtcgagattttcgacatgctgctcgccacctctt |
| | | cccggttccggatgatgaatctgcagggagaagag |
| | | ttcgtgtgtctgaagtcaatcatcctgctgaactc |
| | | cggggtctataccttcctgagctcgaccctcaagt |
| | | cactggaggaaaaagaccacatccatcgcgtgctc |
| | | gataagatcaccgacacccttatccatctcatggc |
| | | gaaggctggactgaccctgcaacagcagcaccaga |
| | | ggctggcccagttgctgctgattctgagccacatc |
| | | cggcacatgtcgaacaagaggatggaacacctgta |
| | | cagcatgaagtgcaagaacgtcgtgcctctgtccg |
| | | atctgctcctggaaatgctggacgcgcacagactc |
| SEQ | FKBP | GVQVETISPGDGRTFPKRGQTCVVHYTGMLEDGKK |
| ID | L106P | VDSSRDRNKPFKFMLGKQEVIRGWEEGVAQMSVGQ |
| NO: | amino | RAKLTISPDYAYGATGHPGIIPPHATLVFDVELLK |
| 346 | acid | PE |
| | se- | |
| | quence | |
| SEQ | DHFR | ISLIAALAVDYVIGMENAMPWNLPADLAWFKRNTL |
| ID | R12Y/ | NKPVIMGRHTWESIGRPLPGRKNIILSSQPSTDDR |
| NO: | G27S/ | VTWVKSVDEAIAACGDVPEIMVIGGGRVIEQFLPK |
| 347 | Y100I | AQKLYLTHIDAEVEGDTHFPDYEPDDWESVFSEFH |
| | amino | DADAQNSHSYCFEILERR |
| | acid | |
| | se- | |
| | quence | |

### Cleavage Site

In some embodiments, the fusion polypeptide, e.g., CCAR, of this disclosure comprises a degradation domain and a heterologous polypeptide, e.g., CAR, separated by a heterologous cleavage site.

The cleavage site can be a protease cleavage site. The cleavage site can be designed to be cleaved by any site-specific protease that is expressed in a cell of interest (either through recombinant expression or endogenous expression) at adequate levels to cleave off the degradation domain. In some embodiments, the protease cleavage site is chosen to correspond to a protease natively (or by virtue of cell engineering) to be present in a cellular compartment relevant to the expression of the protein of interest. The intracellular trafficking of the protease should overlap or partially overlap with the intracellular trafficking of the protein of interest that contains the degradation domain employed. For example, if the protein of interest is located at the cell surface, the enzyme to cleave it can be added exogenously to the cell.

If the protein of interest resides in the endosomal/lysosomal system a protease cleavage site for an enzyme resident in those compartments can be used. Such protease/consensus motifs include, e.g.,

Furin: RX(K/R)R consensus motif (X can be any amino acid; SEQ ID NO: 348)

PCSK1: RX(K/R)R consensus motif (X can be any amino acid; SEQ ID NO: 348)

PCSK5: RX(K/R)R consensus motif (X can be any amino acid; SEQ ID NO: 348)

PCSK6: RX(K/R)R consensus motif (X can be any amino acid; SEQ ID NO: 348)

PCSK7: RXXX[KR]R consensus motif (X can be any amino acid; SEQ ID NO: 349)

Cathepsin B:
(SEQ ID NO: 350)
RRX
Granzyme B:
(SEQ ID NO: 351)
I-E-P-D-X
Factor XA:
(SEQ ID NO: 352)
Ile-Glu/Asp-Gly-Arg
Enterokinase:
(SEQ ID NO: 353)
Asp-Asp-Asp-Asp-Lys
Genenase:
(SEQ ID NO: 354)
Pro-Gly-Ala-Ala-His-Tyr
Sortase:
(SEQ ID NO: 355)
LPXTG/A
PreScission protease:
(SEQ ID NO: 356)
Leu-Glu-Val-Phe-Gln-Gly-Pro
Thrombin:
(SEQ ID NO: 357)
Leu-Val-Pro-Arg-Gly-Ser
TEV protease:
(SEQ ID NO: 358)
E-N-L-Y-F-Q-G

Elastase 1: [AGSV]-X (X can be any amino acid; SEQ ID NO: 359)

In some embodiments, the fusion polypeptide, e.g., CCAR, described herein includes a furin cleavage site. In some embodiments, the fusion polypeptide, e.g., CCAR, described herein includes any one of furin cleavage sites listed in Table 28.

In some embodiments, the fusion polypeptides, e.g., CCARs, described herein include a furin cleavage site selected from RTKR (SEQ ID NO: 378) or a sequence having at least 90%, 95%, 97%, 98%, or 99% identity thereto; GTGAEDPRPSRKRRSLGDVG (SEQ ID NO: 379) or a sequence having at least 90%, 95%, 97%, 98%, or 99% identity thereto; GTGAEDPRPSRKRR (SEQ ID NO: 381) or a sequence having at least 90%, 95%, 97%, 98%, or 99% identity thereto; LQWLEQQVAKRRTKR (SEQ ID NO: 383) or a sequence having at least 90%, 95%, 97%, 98%, or 99% identity thereto; GTGAEDPRPSRKRRSLGG (SEQ ID NO: 385) or a sequence having at least 90%, 95%, 97%, 98%, or 99% identity thereto; GTGAEDPRPSRKRRSLG (SEQ ID NO: 387) or a sequence having at least 90%, 95%, 97%, 98%, or 99% identity thereto; SLNLTESHNSRKKR (SEQ ID NO: 389) or a sequence having at least 90%, 95%, 97%, 98%, or 99% identity thereto; or CKINGYPKRGRKRR (SEQ ID NO: 391) or a sequence having at least 90%, 95%, 97%, 98%, or 99% identity thereto.

In some embodiments, the fusion polypeptide, e.g., CCAR, described herein includes a furin cleavage site selected from RTKR (SEQ ID NO: 378); GTGAEDPRPSRKRRSLGDVG (SEQ ID NO: 379); GTGAEDPRPSRKRR (SEQ ID NO: 381); LQWLEQQVAKRRTKR (SEQ ID NO: 383); GTGAEDPRPSRKRRSLGG (SEQ ID NO: 385); GTGAEDPRPSRKRRSLG (SEQ ID NO: 387); SLNLTESHNSRKKR (SEQ ID NO: 389); or CKINGYPKRGRKRR (SEQ ID NO: 391).

In some embodiments, the fusion polypeptide, e.g., CCAR, described herein includes a furin cleavage site selected from GTGAEDPRPSRKRRSLGDVG (SEQ ID NO: 379) or a sequence having at least 90%, 95%, 97%, 98%, or 99% identity thereto, or GTGAEDPRPSRKRR (SEQ ID NO: 381) or a sequence having at least 90%, 95%, 97%, 98%, or 99% identity thereto.

In some embodiments, the fusion polypeptide, e.g., CCAR, described herein includes a furin cleavage site selected from GTGAEDPRPSRKRRSLGDVG (SEQ ID NO: 379) or GTGAEDPRPSRKRR (SEQ ID NO: 381).

In some embodiments, the fusion polypeptide, e.g., CCAR, described herein includes the furin cleavage site of GTGAEDPRPSRKRRSLGDVG (SEQ ID NO: 379).

TABLE 28
Exemplary furin cleavage site

| | Amino acid | Nucleic acid |
|---|---|---|
| | sequence | sequence |
| Furin | RTKR | cgtactaaaaga |
| cleavage | (SEQ ID NO: 378) | (SEQ ID NO: 393) |
| site 1 | | |
| Furin | GTGAEDPRPSRKRRSL | ggaaccggcgcggaag |
| cleavage | GDVG | acccccggccctccag |
| site 2 | (SEQ ID NO: 379) | gaagcgaaggtccctc |
| | | ggagacgtgggt |
| | | (SEQ ID NO: 380) |
| Furin | GTGAEDPRPSRKRR | ggaaccggcgcggaag |
| cleavage | (SEQ ID NO: 381) | aacccccggccctcca |
| site 3 | | ggaagcgagg |
| | | (SEQ ID NO: 382) |
| Furin | LQWLEQQVAKRRTKR | ctgcaatggctggagc |
| cleavage | (SEQ ID NO: 383) | agcaggtggcgaagcg |
| site 4 | | gagaactaagcgg |
| | | (SEQ ID NO: 384) |
| Furin | GTGAEDPRPSRKRRSL | ggcacaggtgccgagg |
| cleavage | GG | accctcggccaagccg |
| site 5 | (SEQ ID NO: 385) | caaaaggaggtcactt |
| | | ggcggc |
| | | (SEQ ID NO: 386) |
| Furin | GTGAEDPRPSRKRRSL | ggaaccggagcagaag |
| cleavage | G | atcccagaccaagccg |
| site 6 | (SEQ ID NO: 387) | gaaaaggcggtccctg |
| | | ggt |
| | | (SEQ ID NO: 388) |
| Furin | SLNLTESHNSRKKR | agtctcaatttgactg |
| cleavage | (SEQ ID NO: 389) | agtcacacaattccag |
| site 7 | | gaagaaaagg |
| | | (SEQ ID NO: 390) |
| Furin | CKINGYPKRGRKRR | tgcaagatcaacggct |
| cleavage | (SEQ ID NO: 391) | accctaagaggggcag |
| site 8 | | aaagcggcgg |
| | | (SEQ ID NO: 392) |

### Regulatable CAR (RCAR)

In some embodiments, the CCAR described herein can be a regulatable CAR (RCAR). In some embodiments, an RCAR comprises a set of polypeptides, typically two in the simplest embodiments, in which the components of a standard CAR described herein, e.g., an antigen binding domain and an intracellular signaling domain, are partitioned on separate polypeptides or members. In some embodiments, the set of polypeptides include a dimerization switch that, upon the presence of a dimerization molecule, can couple the polypeptides to one another, e.g., can couple an antigen binding domain to an intracellular signaling domain. Additional description and exemplary configurations of such regulatable CARs are provided herein and in International Publication No. WO 2015/090229, hereby incorporated by reference in its entirety.

In an embodiment, an RCAR comprises two polypeptides or members: 1) an intracellular signaling member comprising an intracellular signaling domain, e.g., a primary intracellular signaling domain described herein, and a first switch domain; 2) an antigen binding member comprising an antigen binding domain, e.g., that targets a tumor antigen described herein, as described herein and a second switch domain. Optionally, the RCAR comprises a transmembrane domain described herein. In an embodiment, a transmembrane domain can be disposed on the intracellular signaling member, on the antigen binding member, or on both. (Unless otherwise indicated, when members or elements of an RCAR are described herein, the order can be as provided, but other orders are included as well. In other words, in an embodiment, the order is as set out in the text, but in other embodiments, the order can be different. E.g., the order of elements on one side of a transmembrane region can be different from the example, e.g., the placement of a switch domain relative to a intracellular signaling domain can be different, e.g., reversed).

In an embodiment, the first and second switch domains can form an intracellular or an extracellular dimerization switch. In an embodiment, the dimerization switch can be a homodimerization switch, e.g., where the first and second switch domain are the same, or a heterodimerization switch, e.g., where the first and second switch domain are different from one another.

In embodiments, an RCAR can comprise a “multi switch.” A multi switch can comprise heterodimerization switch domains or homodimerization switch domains. A multi switch comprises a plurality of, e.g., 2, 3, 4, 5, 6, 7, 8, 9, or 10, switch domains, independently, on a first member, e.g., an antigen binding member, and a second member, e.g., an intracellular signaling member. In an embodiment, the first member can comprise a plurality of first switch domains, e.g., FKBP-based switch domains, and the second member can comprise a plurality of second switch domains, e.g., FRB-based switch domains. In an embodiment, the first member can comprise a first and a second switch domain, e.g., a FKBP-based switch domain and a FRB-based switch domain, and the second member can comprise a first and a second switch domain, e.g., a FKBP-based switch domain and a FRB-based switch domain.

In an embodiment, the intracellular signaling member comprises one or more intracellular signaling domains, e.g., a primary intracellular signaling domain and one or more costimulatory signaling domains.

In an embodiment, the antigen binding member may comprise one or more intracellular signaling domains, e.g., one or more costimulatory signaling domains. In an embodiment, the antigen binding member comprises a plurality, e.g., 2 or 3 costimulatory signaling domains described herein, e.g., selected from 4-1BB, CD28, CD27, ICOS, and OX40, and in embodiments, no primary intracellular signaling domain. In an embodiment, the antigen binding member comprises the following costimulatory signaling domains, from the extracellular to intracellular direction: 4-1BB-CD27; 4-1BB-CD27; CD27-4-1BB; 4-1BB-CD28; CD28-4-1BB; OX40-CD28; CD28-OX40; CD28-4-1BB; or 4-1BB-CD28. In such embodiments, the intracellular binding member comprises a CD3zeta domain. In one such embodiment the RCAR comprises (1) an antigen binding member comprising, an antigen binding domain, a transmembrane domain, and two costimulatory domains and a first switch domain; and (2) an intracellular signaling domain comprising a transmembrane domain or membrane tethering domain and at least one primary intracellular signaling domain, and a second switch domain.

An embodiment provides RCARs wherein the antigen binding member is not tethered to the surface of the CAR-expressing cell. This allows a cell having an intracellular signaling member to be conveniently paired with one or more antigen binding domains, without transforming the cell with a sequence that encodes the antigen binding member. In such embodiments, the RCAR comprises: 1) an intracellular signaling member comprising: a first switch domain, a transmembrane domain, an intracellular signaling domain, e.g., a primary intracellular signaling domain, and a first switch domain; and 2) an antigen binding member comprising: an antigen binding domain, and a second switch domain, wherein the antigen binding member does not comprise a transmembrane domain or membrane tethering domain, and, optionally, does not comprise an intracellular signaling domain. In some embodiments, the RCAR may further comprise 3) a second antigen binding member comprising: a second antigen binding domain, e.g., a second antigen binding domain that binds a different antigen than is bound by the antigen binding domain; and a second switch domain.

Also provided herein are RCARs wherein the antigen binding member comprises bispecific activation and targeting capacity. In this embodiment, the antigen binding member can comprise a plurality, e.g., 2, 3, 4, or 5 antigen binding domains, e.g., scFvs, wherein each antigen binding domain binds to a target antigen, e.g. different antigens or the same antigen, e.g., the same or different epitopes on the same antigen. In an embodiment, the plurality of antigen binding domains are in tandem, and optionally, a linker or hinge region is disposed between each of the antigen binding domains. Suitable linkers and hinge regions are described herein.

An embodiment provides RCARs having a configuration that allows switching of proliferation. In this embodiment, the RCAR comprises: 1) an intracellular signaling member comprising: optionally, a transmembrane domain or membrane tethering domain; one or more co-stimulatory signaling domain, e.g., selected from 4-1BB, CD28, CD27, ICOS, and OX40, and a switch domain; and 2) an antigen binding member comprising: an antigen binding domain, a transmembrane domain, and a primary intracellular signaling domain, e.g., a CD3zeta domain, wherein the antigen binding member does not comprise a switch domain, or does not comprise a switch domain that dimerizes with a switch domain on the intracellular signaling member. In an embodiment, the antigen binding member does not comprise a co-stimulatory signaling domain. In an embodiment, the intracellular signaling member comprises a switch domain from a homodimerization switch. In an embodiment, the intracellular signaling member comprises a first switch domain of a heterodimerization switch and the RCAR comprises a second intracellular signaling member which comprises a second switch domain of the heterodimerization switch. In such embodiments, the second intracellular signaling member comprises the same intracellular signaling domains as the intracellular signaling member. In an embodiment, the dimerization switch is intracellular. In an embodiment, the dimerization switch is extracellular.

In any of the RCAR configurations described here, the first and second switch domains comprise a FKBP-FRB based switch as described herein.

Also provided herein are cells comprising an RCAR described herein. Any cell that is engineered to express an RCAR can be used as an RCARX cell. In an embodiment the RCARX cell is a T cell, and is referred to as an RCART cell. In an embodiment the RCARX cell is an NK cell, and is referred to as an RCARN cell.

Also provided herein are nucleic acids and vectors comprising RCAR encoding sequences. Sequence encoding various elements of an RCAR can be disposed on the same nucleic acid molecule, e.g., the same plasmid or vector, e.g., viral vector, e.g., lentiviral vector. In an embodiment, (i) sequence encoding an antigen binding member and (ii) sequence encoding an intracellular signaling member, can be present on the same nucleic acid, e.g., vector. Production of the corresponding proteins can be achieved, e.g., by the use of separate promoters, or by the use of a bicistronic transcription product (which can result in the production of two proteins by cleavage of a single translation product or by the translation of two separate protein products). In an embodiment, a sequence encoding a cleavable peptide, e.g., a P2A or F2A sequence, is disposed between (i) and (ii). In an embodiment, a sequence encoding an IRES, e.g., an EMCV or EV71 IRES, is disposed between (i) and (ii). In these embodiments, (i) and (ii) are transcribed as a single RNA. In an embodiment, a first promoter is operably linked to (i) and a second promoter is operably linked to (ii), such that (i) and (ii) are transcribed as separate mRNAs.

Alternatively, the sequence encoding various elements of an RCAR can be disposed on the different nucleic acid molecules, e.g., different plasmids or vectors, e.g., viral vector, e.g., lentiviral vector. E.g., the (i) sequence encoding an antigen binding member can be present on a first nucleic acid, e.g., a first vector, and the (ii) sequence encoding an intracellular signaling member can be present on the second nucleic acid, e.g., the second vector.

Dimerization Switches

Dimerization switches can be non-covalent or covalent. In a non-covalent dimerization switch, the dimerization molecule promotes a non-covalent interaction between the switch domains. In a covalent dimerization switch, the dimerization molecule promotes a covalent interaction between the switch domains.

In an embodiment, the RCAR comprises a FKBP/FRAP, or FKBP/FRB,-based dimerization switch. FKBP12 (FKBP, or FK506 binding protein) is an abundant cytoplasmic protein that serves as the initial intracellular target for the natural product immunosuppressive drug, rapamycin. Rapamycin binds to FKBP and to the large PI3K homolog FRAP (RAFT, mTOR). FRB is a 93 amino acid portion of FRAP, that is sufficient for binding the FKBP-rapamycin complex (Chen, J., Zheng, X. F., Brown, E. J. & Schreiber, S. L. (1995) Identification of an 11-kDa FKBP12-rapamycin-binding domain within the 289-kDa FKBP12-rapamycin-associated protein and characterization of a critical serine residue. Proc Natl Acad Sci USA 92: 4947-51.)

In embodiments, an FKBP/FRAP, e.g., an FKBP/FRB, based switch can use a dimerization molecule, e.g., rapamycin or a rapamycin analog.

An exemplary amino acid sequence of FKBP is as follows:

(SEQ ID NO: 275)
DVPDYASLGGPSSPKKKRKVSRGVQVETISPGDGRTFPKRGQTCVVHYTG
MLEDGKKFDSSRDRNKPFKFMLGKQEVIRGWEEGVAQMSVGQRAKLTISP
DYAYGATGHPGIIPPHATLVFDVELLKLETSY

In embodiments, an FKBP switch domain can comprise a fragment of FKBP having the ability to bind with FRB, or a fragment or analog thereof, in the presence of rapamycin or a rapalog. In one embodiment, the FKBP switch domain comprises the amino acid sequence of:

(SEQ ID NO: 276)
VQVETISPGDGRTFPKRGQTCVVHYTGMLEDGKKFDSSRDRNKPFKFMLG
KQEVIRGWEEGVAQMSVGQRAKLTISPDYAYGATGHPGIIPPHATLVFDV
ELLKLETS

The amino acid sequence of FRB is as follows:

(SEQ ID NO: 277)
ILWHEMWHEGLEEASRLYFGERNVKGMFEVLEPLHAMMERGPQTLKETSF
NQAYGRDLMEAQEWCRKYMKSGNVKDLTQAWDLYYHVFRRISK

“FKBP/FRAP, e.g., an FKBP/FRB, based switch” as that term is used herein, refers to a dimerization switch comprising: a first switch domain, which comprises an FKBP fragment or analog thereof having the ability to bind with FRB, or a fragment or analog thereof, in the presence of rapamycin or a rapalog, e.g., RAD001, and has at least 70, 75, 80, 85, 90, 95, 96, 97, 98, or 99% identity with, or differs by no more than 30, 25, 20, 15, 10, 5, 4, 3, 2, or 1 amino acid residues from, the FKBP sequence of SEQ ID NO: 275 or 276; and a second switch domain, which comprises an FRB fragment or analog thereof having the ability to bind with FRB, or a fragment or analog thereof, in the presence of rapamycin or a rapalog, and has at least 70, 75, 80, 85, 90, 95, 96, 97, 98, or 99% identity with, or differs by no more than 30, 25, 20, 15, 10, 5, 4, 3, 2, or 1 amino acid residues from, the FRB sequence of SEQ ID NO: 277. In an embodiment, an RCAR described herein comprises one switch domain comprises amino acid residues disclosed in SEQ ID NO: 275 (or SEQ ID NO: 276), and one switch domain comprises amino acid residues disclosed in SEQ ID NO: 277.

In embodiments, the FKBP/FRB dimerization switch comprises a modified FRB switch domain that exhibits altered, e.g., enhanced, complex formation between an FRB-based switch domain, e.g., the modified FRB switch domain, a FKBP-based switch domain, and the dimerization molecule, e.g., rapamycin or a rapalogue, e.g., RAD001. In an embodiment, the modified FRB switch domain comprises one or more mutations, e.g., 2, 3, 4, 5, 6, 7, 8, 9, 10 or more, selected from mutations at amino acid position(s) L2031, E2032, S2035, R2036, F2039, G2040, T2098, W2101, D2102, Y2105, and F2108, where the wild-type amino acid is mutated to any other naturally-occurring amino acid. In an embodiment, a mutant FRB comprises a mutation at E2032, where E2032 is mutated to phenylalanine (E2032F), methionine (E2032M), arginine (E2032R), valine (E2032V), tyrosine (E2032Y), isoleucine (E20321), e.g., SEQ ID NO: 278, or leucine (E2032L), e.g., SEQ ID NO: 279. In an embodiment, a mutant FRB comprises a mutation at T2098, where T2098 is mutated to phenylalanine (T2098F) or leucine (T2098L), e.g., SEQ ID NO: 280. In an embodiment, a mutant FRB comprises a mutation at E2032 and at T2098, where E2032 is mutated to any amino acid, and where T2098 is mutated to any amino acid, e.g., SEQ ID NO: 281. In an embodiment, a mutant FRB comprises an E20321 and a T2098L mutation, e.g., SEQ ID NO: 282. In an embodiment, a mutant FRB comprises an E2032L and a T2098L mutation, e.g., SEQ ID NO: 283.

TABLE 18
Exemplary mutant FRB having increased affinity
for a dimerization molecule.

| | | SEQ |
|---|---|---|
| FRB | | ID |
| mutant | Amino Acid Sequence | NO: |
| E2032I | ILWHEMWHEGLIEASRLYFGERNVKGMFEVLEPLHA | 278 |
| mutant | MMERGPQTLKETSFNQAYGRDLMEAQEWCRKYMKSG | |
| | NVKDLTQAWDLYYHVFRRISKTS | |
| E2032L | ILWHEMWHEGLLEASRLYFGERNVKGMFEVLEPLHA | 279 |
| mutant | MMERGPQTLKETSFNQAYGRDLMEAQEWCRKYMKSG | |
| | NVKDLTQAWDLYYHVFRRISKTS | |
| T2098L | ILWHEMWHEGLEEASRLYFGERNVKGMFEVLEPLHA | 280 |
| mutant | MMERGPQTLKETSFNQAYGRDLMEAQEWCRKYMKSG | |
| | NVKDLLQAWDLYYHVFRRISKTS | |
| E2032, | ILWHEMWHEGLXEASRLYFGERNVKGMFEVLEPLHA | 281 |
| T2098 | MMERGPQTLKETSFNQAYGRDLMEAQEWCRKYMKSG | |
| mutant | NVKDLXQAWDLYYHVFRRISKTS, | |
| | wherein X is any amino acid residue | |
| E2032I, | ILWHEMWHEGLXEASRLYFGERNVKGMFEVLEPLHA | 282 |
| T2098L | MMERGPQTLKETSFNQAYGRDLMEAQEWCRKYMKSG | |
| mutant | NVKDLLQAWDLYYHVFRRISKTS | |
| E2032L, | ILWHEMWHEGLLEASRLYFGERNVKGMFEVLEPLHA | 283 |
| T2098L | MMERGPQTLKETSFNQAYGRDLMEAQEWCRKYMKSG | |
| mutant | NVKDLLQAWDLYYHVFRRISKTS | |

Other suitable dimerization switches include a GyrB-GyrB based dimerization switch, a Gibberellin-based dimerization switch, a tag/binder dimerization switch, and a halo-tag/snap-tag dimerization switch. Following the guidance provided herein, such switches and relevant dimerization molecules will be apparent to one of ordinary skill.

Dimerization Molecule

Association between the switch domains is promoted by the dimerization molecule. In the presence of dimerization molecule interaction or association between switch domains allows for signal transduction between a polypeptide associated with, e.g., fused to, a first switch domain, and a polypeptide associated with, e.g., fused to, a second switch domain. In the presence of non-limiting levels of dimerization molecule signal transduction is increased by 1.1, 1.2, 1.3, 1.4, 1.5, 1.6, 1.7, 1.8, 1.9, 2, 5, 10, 50, 100 fold, e.g., as measured in a system described herein.

Rapamycin and rapamycin analogs (sometimes referred to as rapalogues), e.g., RAD001, can be used as dimerization molecules in a FKBP/FRB-based dimerization switch described herein. In an embodiment the dimerization molecule can be selected from rapamycin (sirolimus), RAD001 (everolimus), zotarolimus, temsirolimus, AP-23573 (ridaforolimus), biolimus and AP21967. Additional rapamycin analogs suitable for use with FKBP/FRB-based dimerization switches are further described in the section entitled “Combination Therapies”, or in the subsection entitled “Combination with a Low, Immune Enhancing, Dose of an mTOR inhibitor”.

### Induciable Caspase for Depletion of CAR-Expressing Cells

In some embodiments, inducing apoptosis using, e.g., a caspase fused to a dimerization domain (see, e.g., Di et al., N Engl. J. Med. 2011 Nov. 3; 365(18):1673-1683), can be used as a safety switch in the CAR therapy of the instant disclosure. In some embodiments, CAR-expressing cells can also express an inducible Caspase-9 (iCaspase-9) molecule that, upon administration of a dimerizer drug (e.g., rimiducid (also called AP1903 (Bellicum Pharmaceuticals) or AP20187 (Ariad)) leads to activation of Caspase-9 and apoptosis of the CAR-expressing cells. The iCaspase-9 molecule contains a chemical inducer of dimerization (CID) binding domain that mediates dimerization in the presence of a CID. This results in inducible and selective depletion of CAR-expressing cells. Thus, the iCaspase-9 can provide a safety switch to avoid any toxicity of CAR-expressing cells. See, e.g., Song et al. Cancer Gene Ther. 2008; 15(10):667-75; Clinical Trial Id. No. NCT02107963; Di Stasi et al. N. Engl. J. Med. 2011; 365:1673-83; and Straathof et al., Blood. 2005 Jun. 1; 105(11):4247-54, herein incorporated by reference in their entireties.

In some embodiments, a cell provided herein comprises a nucleic acid molecule encoding a CAR and a nucleic acid molecule encoding an iCaspase-9 molecule. In some embodiments, the iCaspase-9 molecule comprises a chimeric protein comprising (i) a multimeric ligand binding region and (ii) a caspase 9 molecule. In some embodiments, the caspase 9 molecule is a truncated caspase 9. In some embodiments, the caspase 9 molecule lacks the caspase recruitment domain. In some embodiments, the caspase 9 molecule is a caspase 9 polypeptide or a modified caspase 9 polypeptide disclosed in WO2011146862, WO2014164348, or WO2016100236, herein incorporated by reference in their entireties.

As used herein, the term “caspase 9 molecule” includes a naturally existing caspase 9, a truncated version of caspase 9 (e.g., truncated caspase 9 that lacks a Caspase Activation and Recruitment Domain (CARD) domain), and a variant of caspase 9 (e.g., caspase 9 comprising one or more mutations that reduce its basal activity in the absence of a multimeric ligand).

As used herein, the term “multimeric ligand binding region” refers to a ligand binding region that binds to a multimeric ligand. The term “multimeric ligand” includes a dimeric ligand. A dimeric ligand has two binding sites capable of binding to the ligand receptor domain. A variety of pairs of synthetic ligands and receptors can be employed. For example, in some embodiments involving natural receptors, dimeric FK506 can be used with an FKBP12 receptor, dimerized cyclosporin A can be used with the cyclophilin receptor, dimerized estrogen with an estrogen receptor, dimerized glucocorticoids with a glucocorticoid receptor, dimerized tetracycline with the tetracycline receptor, dimerized vitamin D with the vitamin D receptor, and the like. For embodiments involving unnatural receptors, e.g., antibody subunits, modified antibody subunits, single chain antibodies comprised of heavy and light chain variable regions in tandem, separated by a flexible linker domain, or modified receptors, and mutated sequences thereof, and the like, any of a large variety of compounds can be used. A significant characteristic of these ligand units is that each binding site is able to bind the receptor with high affinity and they are able to be dimerized chemically.

In some embodiments, binding of a multimeric ligand to the multimeric ligand binding region leads to oligomerization (e.g., dimerization) of the chimeric protein, which induces activation of the caspase 9 molecule and apoptosis of the cell. In some embodiments, the multimeric ligand binding region is selected from the group consisting of FKBP, cyclophilin receptor, steroid receptor, tetracycline receptor, heavy chain antibody subunit, light chain antibody subunit, single chain antibodies comprised of heavy and light chain variable regions in tandem separated by a flexible linker domain, and mutated sequences thereof. In some embodiments, the multimeric ligand binding region is an FKBP12 region. In some embodiments, the multimeric ligand is an FK506 dimer or a dimeric FK506 analog ligand. In some embodiments, the multimeric ligand is AP1903. In some embodiments, the multimeric ligand binding region is a multimeric ligand binding region disclosed in WO2011146862, WO2014164348, or WO2016100236. In some embodiments, the multimeric ligand is a multimeric ligand disclosed in WO2011146862, WO2014164348, or WO2016100236.

In some embodiments, the iCaspase-9 molecule is encoded by a nucleic acid molecule separate from the CAR-encoding vector(s). In some embodiments, the iCaspase-9 molecule is encoded by the same nucleic acid molecule as the CAR-encoding vector.

### Truncated EGFR for Depletion of CAR-Expressing Cells

In some embodiments, a cell provided herein comprises a nucleic acid molecule encoding a CAR and a nucleic acid molecule encoding a truncated epidermal growth factor receptor (EGFRt). In some embodiments, the EGFRt lacks the membrane distal EGF-binding domain and the cytoplasmic signaling tail, but retains an extracellular epitope. In some embodiments, the EGFRt comprises one or both of an EGFR Domain III and an EGFR Domain IV. In some embodiments, the EGFRt does not comprise 1, 2, 3, or all of: an EGFR Domain I, an EGFR Domain II, an EGFR juxtamembrane domain, and an EGFR tyrosine kinase domain. In some embodiments, the EGFRt is not immunogenic. In some embodiments, the EGFRt does not mediate signaling or trafficking function. In some embodiments, the EGFRt does not bind an endogenous EGFR ligand, e.g., epidermal growth factor (EGF). In some embodiments, the EGFRt comprises an EGFRt sequence disclosed in WO2011056894 or WO2013123061, incorporated herein by reference in their entireties.

In some embodiments, the EGFRt, when expressed in a cell (e.g., a CAR-expressing cell) can be used to mediate depletion, tracking, and/or purification of the cell. In some embodiments, the EGFRt binds to an anti-EGFR-antibody molecule, an EGFR-specific siRNA, or a small molecule that targets EGFR. In some embodiments, the EGFRt binds to an anti-EGFR antibody selected from the group consisting of cetuximab, matuzumab, necitumumab and panitumumab.

In some embodiments, the EGFRt is encoded by a nucleic acid molecule separate from the CAR-encoding vector(s). In some embodiments, the EGFRt is encoded by the same nucleic acid molecule as the CAR-encoding vector.

### Chimeric Antigen Receptor (CAR)

The present disclosure provides immune effector cells (for example, T cells or NK cells) that are engineered to contain one or more CARs, e.g., CCARs, that direct the immune effector cells to cancer. In some embodiments, the immune effector cells are engineered to express a CCAR disclosed herein. In some embodiments, the immune effector cells are engineered to express a CAR disclosed herein and a regulatory molecule disclosed herein.

This is achieved through an antigen binding domain on the CAR that is specific for a cancer associated antigen. There are two classes of cancer associated antigens (tumor antigens) that can be targeted by the CARs described herein: (1) cancer associated antigens that are expressed on the surface of cancer cells; and (2) cancer associated antigens that themselves are intracellular, however, fragments (peptides) of such antigens are presented on the surface of the cancer cells by MHC (major histocompatibility complex).

Accordingly, an immune effector cell, for example, obtained by a method described herein, can be engineered to contain a CAR that targets one of the following cancer associated antigens (tumor antigens): CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, VEGFR2, LewisY, CD24, PDGFR-beta, PRSS21, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGSS, HMWMAA, o-acetyl-GD2, Folate receptor beta, TEM1/CD248, TEM7R, CLDN6, TSHR, GPRC5D, CXORF61, CD97, CD179a, ALK, Plysialic acid, PLAC1, GloboH, NY-BR-1, UPK2, HAVCR1, ADRB3, PANX3, GPR20, LY6K, OR51E2, TARP, WT1, NY-ESO-1, LAGE-1a, legumain, HPV E6,E7, MAGE-A1, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, and mut hsp70-2.

Sequences of non-limiting examples of various components that can be part of a CAR molecule described herein are listed in Table 1, where “aa” stands for amino acids, and “na” stands for nucleic acids that encode the corresponding peptide.

TABLE 1
Sequences of various components of CAR

| SEQ | | |
|---|---|---|
| ID | Descrip | |
| NO | tion | Sequence |
| SEQ | EF-1α | CGTGAGGCTCCGGTGCCCGTCAGTGGGCAGAGCGCACAT |
| ID | promoter | CGCCCACAGTCCCCGAGAAGTTGGGGGGAGGGGTCGGC |
| NO: | (na) | AATTGAACCGGTGCCTAGAGAAGGTGGCGCGGGGTAAA |
| 11 | | CTGGGAAAGTGATGTCGTGTACTGGCTCCGCCTTTTTCCC |
| | | GAGGGTGGGGGAGAACCGTATATAAGTGCAGTAGTCGC |
| | | CGTGAACGTTCTTTTTCGCAACGGGTTTGCCGCCAGAAC |
| | | ACAGGTAAGTGCCGTGTGTGGTTCCCGCGGGCCTGGCCT |
| | | CTTTACGGGTTATGGCCCTTGCGTGCCTTGAATTACTTCC |
| | | ACCTGGCTGCAGTACGTGATTCTTGATCCCGAGCTTCGG |
| | | GTTGGAAGTGGGTGGGAGAGTTCGAGGCCTTGCGCTTAA |
| | | GGAGCCCCTTCGCCTCGTGCTTGAGTTGAGGCCTGGCCT |
| | | GGGCGCTGGGGCCGCCGCGTGCGAATCTGGTGGCACCTT |
| | | CGCGCCTGTCTCGCTGCTTTCGATAAGTCTCTAGCCATTT |
| | | AAAATTTTTGATGACCTGCTGCGACGCTTTTTTTCTGGCA |
| | | AGATAGTCTTGTAAATGCGGGCCAAGATCTGCACACTGG |
| | | TATTTCGGTTTTTGGGGCCGCGGGCGGCGACGGGGCCCG |
| | | TGCGTCCCAGCGCACATGTTCGGCGAGGCGGGGCCTGCG |
| | | AGCGCGGCCACCGAGAATCGGACGGGGGTAGTCTCAAG |
| | | CTGGCCGGCCTGCTCTGGTGCCTGGCCTCGCGCCGCCGT |
| | | GTATCGCCCCGCCCTGGGCGGCAAGGCTGGCCCGGTCGG |
| | | CACCAGTTGCGTGAGCGGAAAGATGGCCGCTTCCCGGCC |
| | | CTGCTGCAGGGAGCTCAAAATGGAGGACGCGGCGCTCG |
| | | GGAGAGCGGGCGGGTGAGTCACCCACACAAAGGAAAAG |
| | | GGCCTTTCCGTCCTCAGCCGTCGCTTCATGTGACTCCACG |
| | | GAGTACCGGGCGCCGTCCAGGCACCTCGATTAGTTCTCG |
| | | AGCTTTTGGAGTACGTCGTCTTTAGGTTGGGGGGAGGGG |
| | | TTTTATGCGATGGAGTTTCCCCACACTGAGTGGGTGGAG |
| | | ACTGAAGTTAGGCCAGCTTGGCACTTGATGTAATTCTCC |
| | | TTGGAATTTGCCCTTTTTGAGTTTGGATCTTGGTTCATTC |
| | | TCAAGCCTCAGACAGTGGTTCAAAGTTTTTTTCTTCCATT |
| | | TCAGGTGTCGTGA |
| SEQ | Leader | MALPVTALLLPLALLLHAARP |
| ID | (aa) | |
| NO: | | |
| 1 | | |
| SEQ | Leader | ATGGCCCTGCCTGTGACAGCCCTGCTGCTGCCTCTGGCTC |
| ID | (na) | TGCTGCTGCATGCCGCTAGACCC |
| NO: | | |
| 12 | | |
| SEQ | Leader | ATGGCCCTCCCTGTCACCGCCCTGCTGCTTCCGCTGGCTC |
| ID | (na) | TTCTGCTCCACGCCGCTCGGCCC |
| NO: | | |
| 199 | | |
| SEQ | Leader | MLLLVTSLLLCELPHPAFLLIP |
| ID | (aa) | |
| NO: | | |
| 394 | | |
| SEQ | Leader | ATGCTTCTCCTGGTGACAAGCCTTCTGCTCTGTGAGTTAC |
| ID | (na) | CACACCCAGCATTCCTCCTGATCCCA |
| NO: | | |
| 395 | | |
| SEQ | Leader | ATGCTGCTGCTGGTGACCAGCCTGCTGCTGTGCGAGCTG |
| ID | (na) | CCCCACCCCGCCTTTCTGCTGATCCCC |
| NO: | | |
| 396 | | |
| SEQ | CD 8 | TTTPAPRPPTPAPTIASQPLSLRPEACRPAAGGAVHTRGLDF |
| ID | hinge | ACD |
| NO: | (aa) | |
| 2 | | |
| SEQ | CD 8 | ACCACGACGCCAGCGCCGCGACCACCAACACCGGCGCC |
| ID | hinge | CACCATCGCGTCGCAGCCCCTGTCCCTGCGCCCAGAGGC |
| NO: | (na) | GTGCCGGCCAGCGGCGGGGGGCGCAGTGCACACGAGGG |
| 13 | | GGCTGGACTTCGCCTGTGAT |
| SEQ | Ig4 | ESKYGPPCPPCPAPEFLGGPSVFLFPPKPKDTLMISRTPEVTC |
| ID | hinge | VVVDVSQEDPEVQFNWYVDGVEVHNAKTKPREEQFNSTY |
| NO: | (aa) | RVVSVLTVLHQDWLNGKEYKCKVSNKGLPSSIEKTISKAK |
| 3 | | GQPREPQVYTLPPSQEEMTKNQVSLTCLVKGFYPSDIAVEW |
| | | ESNGQPENNYKTTPPVLDSDGSFFLYSRLTVDKSRWQEGN |
| | | VFSCSVMHEALHNHYTQKSLSLSLGKM |
| SEQ | Ig4 | GAGAGCAAGTACGGCCCTCCCTGCCCCCCTTGCCCTGCC |
| ID | hinge | CCCGAGTTCCTGGGCGGACCCAGCGTGTTCCTGTTCCCC |
| NO: | (na) | CCCAAGCCCAAGGACACCCTGATGATCAGCCGGACCCCC |
| 14 | | GAGGTGACCTGTGTGGTGGTGGACGTGTCCCAGGAGGAC |
| | | CCCGAGGTCCAGTTCAACTGGTACGTGGACGGCGTGGAG |
| | | GTGCACAACGCCAAGACCAAGCCCCGGGAGGAGCAGTT |
| | | CAATAGCACCTACCGGGTGGTGTCCGTGCTGACCGTGCT |
| | | GCACCAGGACTGGCTGAACGGCAAGGAATACAAGTGTA |
| | | AGGTGTCCAACAAGGGCCTGCCCAGCAGCATCGAGAAA |
| | | ACCATCAGCAAGGCCAAGGGCCAGCCTCGGGAGCCCCA |
| | | GGTGTACACCCTGCCCCCTAGCCAAGAGGAGATGACCAA |
| | | GAACCAGGTGTCCCTGACCTGCCTGGTGAAGGGCTTCTA |
| | | CCCCAGCGACATCGCCGTGGAGTGGGAGAGCAACGGCC |
| | | AGCCCGAGAACAACTACAAGACCACCCCCCCTGTGCTGG |
| | | ACAGCGACGGCAGCTTCTTCCTGTACAGCCGGCTGACCG |
| | | TGGACAAGAGCCGGTGGCAGGAGGGCAACGTCTTTAGC |
| | | TGCTCCGTGATGCACGAGGCCCTGCACAACCACTACACC |
| | | CAGAAGAGCCTGAGCCTGTCCCTGGGCAAGATG |
| SEQ | IgD | RWPESPKAQASSVPTAQPQAEGSLAKATTAPATTRNTGRG |
| ID | hinge | GEEKKKEKEKEEQEERETKTPECPSHTQPLGVYLLTPAVQD |
| NO: | (aa) | LWLRDKATFTCFVVGSDLKDAHLTWEVAGKVPTGGVEEG |
| 4 | | LLERHSNGSQSQHSRLTLPRSLWNAGTSVTCTLNHPSLPPQ |
| | | RLMALREPAAQAPVKLSLNLLASSDPPEAASWLLCEVSGFS |
| | | PPNILLMWLEDQREVNTSGFAPARPPPQPGSTTFWAWSVLR |
| | | VPAPPSPQPATYTCVVSHEDSRTLLNASRSLEVSYVTDH |
| SEQ | IgD | AGGTGGCCCGAAAGTCCCAAGGCCCAGGCATCTAGTGTT |
| ID | hinge | CCTACTGCACAGCCCCAGGCAGAAGGCAGCCTAGCCAA |
| NO: | (na) | AGCTACTACTGCACCTGCCACTACGCGCAATACTGGCCG |
| 15 | | TGGCGGGGAGGAGAAGAAAAAGGAGAAAGAGAAAGAA |
| | | GAACAGGAAGAGAGGGAGACCAAGACCCCTGAATGTCC |
| | | ATCCCATACCCAGCCGCTGGGCGTCTATCTCTTGACTCCC |
| | | GCAGTACAGGACTTGTGGCTTAGAGATAAGGCCACCTTT |
| | | ACATGTTTCGTCGTGGGCTCTGACCTGAAGGATGCCCAT |
| | | TTGACTTGGGAGGTTGCCGGAAAGGTACCCACAGGGGG |
| | | GGTTGAGGAAGGGTTGCTGGAGCGCCATTCCAATGGCTC |
| | | TCAGAGCCAGCACTCAAGACTCACCCTTCCGAGATCCCT |
| | | GTGGAACGCCGGGACCTCTGTCACATGTACTCTAAATCA |
| | | TCCTAGCCTGCCCCCACAGCGTCTGATGGCCCTTAGAGA |
| | | GCCAGCCGCCCAGGCACCAGTTAAGCTTAGCCTGAATCT |
| | | GCTCGCCAGTAGTGATCCCCCAGAGGCCGCCAGCTGGCT |
| | | CTTATGCGAAGTGTCCGGCTTTAGCCCGCCCAACATCTT |
| | | GCTCATGTGGCTGGAGGACCAGCGAGAAGTGAACACCA |
| | | GCGGCTTCGCTCCAGCCCGGCCCCCACCCCAGCCGGGTT |
| | | CTACCACATTCTGGGCCTGGAGTGTCTTAAGGGTCCCAG |
| | | CACCACCTAGCCCCCAGCCAGCCACATACACCTGTGTTG |
| | | TGTCCCATGAAGATAGCAGGACCCTGCTAAATGCTTCTA |
| | | GGAGTCTGGAGGTTTCCTACGTGACTGACCATT |
| SEQ | CD8 | IYIWAPLAGTCGVLLLSLVITLYC |
| ID | Trans- | |
| NO: | membrane | |
| 6 | (aa) | |
| SEQ | CD8 | ATCTACATCTGGGCGCCCTTGGCCGGGACTTGTGGGGTC |
| ID | Trans- | CTTCTCCTGTCACTGGTTATCACCCTTTACTGC |
| NO: | membrane | |
| 17 | (na) | |
| SEQ | 4-1BB | KRGRKKLLYIFKQPFMRPVQTTQEEDGCSCRFPEEEEGGCE |
| ID | intra- | L |
| NO: | cellular | |
| 7 | domain | |
| | (aa) | |
| SEQ | 4-1BB | AAACGGGGCAGAAAGAAACTCCTGTATATATTCAAACA |
| ID | intra- | ACCATTTATGAGACCAGTACAAACTACTCAAGAGGAAG |
| NO: | cellular | ATGGCTGTAGCTGCCGATTTCCAGAAGAAGAAGAAGGA |
| 18 | domain | GGATGTGAACTG |
| | (na) | |
| SEQ | CD27 | QRRKYRSNKGESPVEPAEPCRYSCPREEEGSTIPIQEDYRKP |
| ID | (aa) | EPACSP |
| NO: | | |
| 8 | | |
| SEQ | CD27 | AGGAGTAAGAGGAGCAGGCTCCTGCACAGTGACTACAT |
| ID | (na) | GAACATGACTCCCCGCCGCCCCGGGCCCACCCGCAAGCA |
| NO: | | TTACCAGCCCTATGCCCCACCACGCGACTTCGCAGCCTA |
| 19 | | TCGCTCC |
| SEQ | CD3-zeta | RVKFSRSADAPAYKQGQNQLYNELNLGRREEYDVLDKRR |
| ID | (aa) | GRDPEMGGKPRRKNPQEGLYNELQKDKMAEAYSEIGMKG |
| NO: | (Q/K | ERRRGKGHDGLYQGLSTATKDTYDALHMQALPPR |
| 9 | mutant) | |
| SEQ | CD3-zeta | AGAGTGAAGTTCAGCAGGAGCGCAGACGCCCCCGCGTA |
| ID | (na) | CAAGCAGGGCCAGAACCAGCTCTATAACGAGCTCAATCT |
| NO: | (Q/K | AGGACGAAGAGAGGAGTACGATGTTTTGGACAAGAGAC |
| 20 | mutant) | GTGGCCGGGACCCTGAGATGGGGGGAAAGCCGAGAAGG |
| | | AAGAACCCTCAGGAAGGCCTGTACAATGAACTGCAGAA |
| | | AGATAAGATGGCGGAGGCCTACAGTGAGATTGGGATGA |
| | | AAGGCGAGCGCCGGAGGGGCAAGGGGCACGATGGCCTT |
| | | TACCAGGGTCTCAGTACAGCCACCAAGGACACCTACGAC |
| | | GCCCTTCACATGCAGGCCCTGCCCCCTCGC |
| SEQ | CD3-zeta | RVKFSRSADAPAYQQGQNQLYNELNLGRREEYDVLDKRR |
| ID | (aa) | GRDPEMGGKPRRKNPQEGLYNELQKDKMAEAYSEIGMKG |
| NO: | (NCBI | ERRRGKGHDGLYQGLSTATKDTYDALHMQALPPR |
| 10 | Reference | |
| | Sequence | |
| | NM_ | |
| | 000734.3) | |
| SEQ | CD3-zeta | AGAGTGAAGTTCAGCAGGAGCGCAGACGCCCCCGCGTA |
| ID | (na) | CCAGCAGGGCCAGAACCAGCTCTATAACGAGCTCAATCT |
| NO: | (NCBI | AGGACGAAGAGAGGAGTACGATGTTTTGGACAAGAGAC |
| 21 | Reference | GTGGCCGGGACCCTGAGATGGGGGGAAAGCCGAGAAGG |
| | Sequence | AAGAACCCTCAGGAAGGCCTGTACAATGAACTGCAGAA |
| | NM_ | AGATAAGATGGCGGAGGCCTACAGTGAGATTGGGATGA |
| | 000734.3) | AAGGCGAGCGCCGGAGGGGCAAGGGGCACGATGGCCTT |
| | | TACCAGGGTCTCAGTACAGCCACCAAGGACACCTACGAC |
| | | GCCCTTCACATGCAGGCCCTGCCCCCTCGC |
| SEQ | CD28 | RSKRSRLLHSDYMNMTPRRPGPTRKHYQPYAPPRDFAAYR |
| ID | Intra- | S |
| NO: | cellular | |
| 36 | domain | |
| | (amino | |
| | acid | |
| | sequence) | |
| SEQ | CD28 | AGGAGTAAGAGGAGCAGGCTCCTGCACAGTGACTACAT |
| ID | Intra | GAACATGACTCCCCGCCGCCCCGGGCCCACCCGCAAGCA |
| NO: | cellular | TTACCAGCCCTATGCCCCACCACGCGACTTCGCAGCCTA |
| 37 | domain | TCGCTCC |
| | (nucleo- | |
| | tide | |
| | sequence) | |
| SEQ | ICOS | T K K K Y S S S V H D P N G E Y M F M |
| ID | Intra- | R A V N T A K K S R L T D V T L |
| NO: | cellular | |
| 38 | domain | |
| | (amino | |
| | acid | |
| | sequence) | |
| SEQ | ICOS | ACAAAAAAGAAGTATTCATCCAGTGTGCACGACCCTAAC |
| ID | Intra- | GGTGAATACATGTTCATGAGAGCAGTGAACACAGCCAA |
| NO: | cellular | AAAATCCAGACTCACAGATGTGACCCTA |
| 39 | domain | |
| | (nucleo- | |
| | tide | |
| | sequence) | |
| SEQ | GS hinge/ | GGGGSGGGGS |
| ID | linker | |
| NO: | (aa) | |
| 5 | | |
| SEQ | GS hinge/ | GGTGGCGGAGGTTCTGGAGGTGGAGGTTCC |
| ID | linker | |
| NO: | (na) | |
| 16 | | |
| SEQ | GS hinge/ | GGTGGCGGAGGTTCTGGAGGTGGGGGTTCC |
| ID | linker | |
| NO: | (na) | |
| 40 | | |
| SEQ | linker | GGGGS |
| ID | | |
| NO: | | |
| 25 | | |
| SEQ | linker | (Gly-Gly-Gly-Gly-Ser)n, where n = 1-6, for example, |
| ID | | GGGGSGGGGS GGGGSGGGGS GGGGSGGGGS |
| NO: | | |
| 26 | | |
| SEQ | linker | GGGGSGGGGSGGGGSGGGGS |
| ID | | |
| NO: | | |
| 27 | | |
| SEQ | linker | GGGGSGGGGSGGGGS |
| ID | | |
| NO: | | |
| 28 | | |
| SEQ | linker | GGGS |
| ID | | |
| NO: | | |
| 29 | | |
| SEQ | linker | (Gly-Gly-Gly-Ser)n where n is a positive integer |
| ID | | equal to or greater than 1 |
| NO: | | |
| 41 | | |
| SEQ | linker | (Gly-Gly-Gly-Ser)n, where n = 1-10, for example, |
| ID | | GGGSGGGSGG GSGGGSGGGS GGGSGGGSGG |
| NO: | | GSGGGSGGGS |
| 42 | | |
| SEQ | linker | GSTSGSGKPGSGEGSTKG |
| ID | | |
| NO: | | |
| 43 | | |
| SEQ | poly(A) | (A)₅₀₀₀ |
| ID | | This sequence may encompass 50-5000 adenines. |
| NO: | | |
| 30 | | |
| SEQ | polyT | (T)₁₀₀ |
| ID | | |
| NO: | | |
| 31 | | |
| SEQ | polyT | (T)₅₀₀₀ |
| ID | | This sequence may encompass 50-5000 thymines. |
| NO: | | |
| 32 | | |
| SEQ | poly(A) | (A)₅₀₀₀ |
| ID | | This sequence may encompass 100-5000 adenines. |
| NO: | | |
| 33 | | |
| SEQ | poly(A) | (A)₄₀₀ |
| ID | | This sequence may encompass 100-400 adenines. |
| NO: | | |
| 34 | | |
| SEQ | poly(A) | (A)₂₀₀₀ |
| ID | | This sequence may encompass 50-2000 adenines. |
| NO: | | |
| 35 | | |
| SEQ | PD1 | pgwfldspdrpwnpptfspallvvtegdnatftcsfsntsesfvlnwyrmspsnqtdklaaf |
| ID | CAR(aa) | pedrsqpgqdcrfrvtqlpngrdfhmsvvrarrndsgtylcgaislapkaqikeslraelrvt |
| NO: | | erraevptahpspsprpagqfqtlvtttpaprpptpaptiasqplslrpeacrpaaggavhtrg |
| 22 | | ldfacdiyiwaplagtcgvlllslvitlyckrgrkkllyifkqpfmrpvqttqeedgcscrfpe |
| | | eeeggcelrvkfsrsadapaykqgqnqlynelnlgrreeydvldkrrgrdpemggkprrk |
| | | npqeglynelqkdkmaeayseigmkgerrrgkghdglyqglstatkdtydalhmqalpp |
| | | r |
| SEQ | PD-1 | atggccctccctgtcactgccctgcttctccccctcgcactcctgctccacgccgctagaccac |
| ID | CAR(na) | ccggatggtttctggactctccggatcgcccgtggaatcccccaaccttctcaccggcactctt |
| NO: | (PD1 ECD | ggttgtgactgagggcgataatgcgaccttcacgtgctcgttctccaacacctccgaatcattc |
| 23 | under- | gtgctgaactggtaccgcatgagcccgtcaaaccagaccgacaagctcgccgcgtttccgga |
| | lined) | agatcggtcgcaaccgggacaggattgtcggttccgcgtgactcaactgccgaatggcagag |
| | | acttccacatgagcgtggtccgcgctaggcgaaacgactccgggacctacctgtgcggagc |
| | | catctcgctggcgcctaaggcccaaatcaaagagagcttgagggccgaactgagagtgacc |
| | | gagcgcagagctgaggtgccaactgcacatccatccccatcgcctcggcctgcggggcagt |
| | | ttcagaccctggtcacgaccactccggcgccgcgcccaccgactccggccccaactatcgc |
| | | gagccagcccctgtcgctgaggccggaagcatgccgccctgccgccggaggtgctgtgcat |
| | | acccggggattggacttcgcatgcgacatctacatttgggctcctctcgccggaacttgtggcg |
| | | tgctccttctgtccctggtcatcaccctgtactgcaagcggggtcggaaaaagcttctgtacat |
| | | tttcaagcagcccttcatgaggcccgtgcaaaccacccaggaggaggacggttgctcctgccg |
| | | gttccccgaagaggaagaaggaggttgcgagctgcgcgtgaagttctcccggagcgccgac |
| | | gcccccgcctataagcagggccagaaccagctgtacaacgaactgaacctgggacggcgg |
| | | gaagagtacgatgtgctggacaagcggcgcggccgggaccccgaaatgggcgggaagcc |
| | | tagaagaaagaaccctcaggaaggcctgtataacgagctgcagaaggacaagatggccga |
| | | ggcctactccgaaattgggatgaagggagagcggcggaggggaaaggggcacgacggcc |
| | | tgtaccaaggactgtccaccgccaccaaggacacatacgatgccctgcacatgcaggccctt |
| | | ccccctcgc |
| SEQ | PD-1 | Malpvtalllplalllhaarppgwfldspdrpwnpptfspallvvtegdnatftcsfsntsesf |
| ID | CAR(aa) | vlnwyrmspsnqtdklaafpedrsqpgqdcrfrvtqlpngrdfhmsvvrarrndsgtylc |
| NO: | (PD1 ECD | gaislapkaqikeslraelrvterraevptahpspsprpagqfqtlvtttpaprpptpaptias |
| 24 | under- | qplslrpeacrpaaggavhtrgldfacdiyiwaplagtcgvlllslvitlyckrgrkkllyifk |
| | lined) | qpfmrpvqttqeedgcscrfpeeeeggcelrvkfsrsadapaykqgqnqlynelnlgrreeyd |
| | with | vldkrrgrdpemggkprrknpqeglynelqkdkmaeayseigmkgerrrgkghdglyq |
| | signal | glstatkdtydalhmqalppr |

In some embodiments the antigen binding domain comprises the extracellular domain, or a counter-ligand binding fragment thereof, of molecule that binds a counterligand on the surface of a target cell.

The immune effector cells can comprise a recombinant DNA construct comprising sequences encoding a CAR, e.g., a CCAR, wherein the CAR comprises an antigen binding domain (for example, antibody or antibody fragment, TCR or TCR fragment) that binds specifically to a tumor antigen, for example, a tumor antigen described herein, and an intracellular signaling domain. The intracellular signaling domain can comprise a costimulatory signaling domain and/or a primary signaling domain, for example, a zeta chain. As described elsewhere, the methods described herein can include transducing a cell, for example, from the population of T regulatory-depleted cells, with a nucleic acid encoding a CAR, for example, a CCAR described herein.

In some embodiments, a CAR comprises a scFv domain, wherein the scFv may be preceded by an optional leader sequence such as provided in SEQ ID NO: 1, and followed by an optional hinge sequence such as provided in SEQ ID NO:2 or SEQ ID NO:36 or SEQ ID NO:38, a transmembrane region such as provided in SEQ ID NO:6, an intracellular signaling domain that includes SEQ ID NO:7 or SEQ ID NO:16 and a CD3 zeta sequence that includes SEQ ID NO:9 or SEQ ID NO:10, for example, wherein the domains are contiguous with and in the same reading frame to form a single fusion protein.

In some embodiments, an exemplary CAR constructs comprise an optional leader sequence (for example, a leader sequence described herein), an extracellular antigen binding domain (for example, an antigen binding domain described herein), a hinge (for example, a hinge region described herein), a transmembrane domain (for example, a transmembrane domain described herein), and an intracellular stimulatory domain (for example, an intracellular stimulatory domain described herein). In some embodiments, an exemplary CAR construct comprises an optional leader sequence (for example, a leader sequence described herein), an extracellular antigen binding domain (for example, an antigen binding domain described herein), a hinge (for example, a hinge region described herein), a transmembrane domain (for example, a transmembrane domain described herein), an intracellular costimulatory signaling domain (for example, a costimulatory signaling domain described herein) and/or an intracellular primary signaling domain (for example, a primary signaling domain described herein).

An exemplary leader sequence is provided as SEQ ID NO: 1. An exemplary hinge/spacer sequence is provided as SEQ ID NO: 2 or SEQ ID NO:36 or SEQ ID NO:38. An exemplary transmembrane domain sequence is provided as SEQ ID NO:6. An exemplary sequence of the intracellular signaling domain of the 4-1BB protein is provided as SEQ ID NO: 7. An exemplary sequence of the intracellular signaling domain of CD27 is provided as SEQ ID NO:16. An exemplary CD3zeta domain sequence is provided as SEQ ID NO: 9 or SEQ ID NO:10.

In some embodiments, the immune effector cell comprises a recombinant nucleic acid construct comprising a nucleic acid molecule encoding a CAR, wherein the nucleic acid molecule comprises a nucleic acid sequence encoding an antigen binding domain, wherein the sequence is contiguous with and in the same reading frame as the nucleic acid sequence encoding an intracellular signaling domain. An exemplary intracellular signaling domain that can be used in the CAR includes, but is not limited to, one or more intracellular signaling domains of, for example, CD3-zeta, CD28, CD27, 4-1BB, and the like. In some instances, the CAR can comprise any combination of CD3-zeta, CD28, 4-1BB, and the like.

The nucleic acid sequences coding for the desired molecules can be obtained using recombinant methods known in the art, such as, for example by screening libraries from cells expressing the nucleic acid molecule, by deriving the nucleic acid molecule from a vector known to include the same, or by isolating directly from cells and tissues containing the same, using standard techniques. Alternatively, the nucleic acid of interest can be produced synthetically, rather than cloned.

Nucleic acids encoding a CAR can be introduced into the immune effector cells using, for example, a retroviral or lentiviral vector construct.

Nucleic acids encoding a CAR can also be introduced into the immune effector cell using, for example, an RNA construct that can be directly transfected into a cell. A method for generating mRNA for use in transfection involves in vitro transcription (IVT) of a template with specially designed primers, followed by poly(A) addition, to produce a construct containing 3′ and 5′ untranslated sequence (“UTR”) (for example, a 3′ and/or 5′ UTR described herein), a 5′ cap (for example, a 5′ cap described herein) and/or Internal Ribosome Entry Site (IRES) (for example, an IRES described herein), the nucleic acid to be expressed, and a poly(A) tail, typically 50-2000 bases in length (for example, described in the Examples, for example, SEQ ID NO:35). RNA so produced can efficiently transfect different kinds of cells. In some embodiments, the template includes sequences for the CAR. In some embodiments, an RNA CAR vector is transduced into a cell, for example, a T cell by electroporation.

### Antigen Binding Domain

In some embodiments, a plurality of the immune effector cells, for example, the population of T regulatory-depleted cells, include a nucleic acid encoding a CAR (e.g., a CCAR) that comprises a target-specific binding element otherwise referred to as an antigen binding domain. The choice of binding element depends upon the type and number of ligands that define the surface of a target cell. For example, the antigen binding domain may be chosen to recognize a ligand that acts as a cell surface marker on target cells associated with a particular disease state. Thus, examples of cell surface markers that may act as ligands for the antigen binding domain in a CAR described herein include those associated with viral, bacterial and parasitic infections, autoimmune disease and cancer cells.

In some embodiments, the portion of the CAR (e.g., a CCAR) comprising the antigen binding domain comprises an antigen binding domain that targets a tumor antigen, for example, a tumor antigen described herein.

The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, a T cell receptor (TCR), or a fragment there of, for example, single chain TCR, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment.

### CD19 CAR

In some embodiments, the CAR-expressing cell described herein is a CD19 CAR-expressing cell (for example, a cell expressing a CAR that binds to human CD19).

In some embodiments, the antigen binding domain of the CD19 CAR has the same or a similar binding specificity as the FMC63 scFv fragment described in Nicholson et al. Mol. Immun. 34 (16-17): 1157-1165 (1997). In some embodiments, the antigen binding domain of the CD19 CAR includes the scFv fragment described in Nicholson et al. Mol. Immun. 34 (16-17): 1157-1165 (1997).

In some embodiments, the CD19 CAR includes an antigen binding domain (for example, a humanized antigen binding domain) according to Table 3 of WO2014/153270, incorporated herein by reference. WO2014/153270 also describes methods of assaying the binding and efficacy of various CAR constructs.

In some embodiments, the parental murine scFv sequence is the CAR19 construct provided in PCT publication WO2012/079000 (incorporated herein by reference). In some embodiments, the anti-CD19 binding domain is a scFv described in WO2012/079000.

In some embodiments, the CAR molecule comprises the fusion polypeptide sequence provided as SEQ ID NO: 12 in PCT publication WO2012/079000, which provides an scFv fragment of murine origin that specifically binds to human CD19.

In some embodiments, the CD19 CAR comprises an amino acid sequence provided as SEQ ID NO: 12 in PCT publication WO2012/079000.

In some embodiments, the amino acid sequence is:

Diqmtqttsslsaslgdrvtiscrasqdiskylnwyqqkpdgtvklliyhtsrlhsgvpsrfsgsgsgtdysltisnleqediatyfcqqgn tlpytfgggtkleitggggsggggsggggsevklqesgpglvapsqslsvtctvsgvslpdygvswirqpprkglewlgviwgsettyynsalksr ltiikdnsksqvflkmnslqtddtaiyycakhyyyggsyamdywgqgtsvtvsstttpaprpptpaptiasqplslrpeacrpaaggavhtrgldfa cdiyiwaplagtcgvlllslvitlyckrgrkkllyifkqpfmrpvqttqeedgcscrfpeeeeggcelrvkfsrsadapaykqgqnqlynelnlgrre eydvldkagrdpemggkprrknpqeglynelqkdkmaeayseigmkgeragkghdglyqglstatkdtydalhmqalppr (SEQ ID NO: 292), or a sequence substantially homologous thereto.

In some embodiments, the CD19 CAR has the USAN designation TISAGENLECLEUCEL-T. In embodiments, CTL019 is made by a gene modification of T cells is mediated by stable insertion via transduction with a self-inactivating, replication deficient Lentiviral (LV) vector containing the CTL019 transgene under the control of the EF-1 alpha promoter. CTL019 can be a mixture of transgene positive and negative T cells that are delivered to the subject on the basis of percent transgene positive T cells.

In one embodiment, the CART cell that specifically binds to CD19 has the INN designation Axicabtagene ciloleucel. In one embodiment, the CART cell that specifically binds to CD19 has the USAN designation brexucabtagene autoleucel. In some embodiments, Axicabtagene ciloleucel is also known as YESCARTA®, Axi-cel, or KTE-C19. In some embodiments, brexucabtagene autoleucel is also known as KTE-X19 or TECARTUS ®.

In one embodiment, the CART cell that specifically binds to CD19 has the INN designation Lisocabtagene maraleucel. In some embodiments, Lisocabtagene maraleucel is also known as JCAR017.

In other embodiments, the CD19 CAR comprises an antigen binding domain (for example, a humanized antigen binding domain) according to Table 3 of WO2014/153270, incorporated herein by reference.

Humanization of murine CD19 antibody is desired for the clinical setting, where the mouse-specific residues may induce a human-anti-mouse antigen (HAMA) response in patients who receive CART19 treatment, i.e., treatment with T cells transduced with the CAR19 construct. The production, characterization, and efficacy of humanized CD19 CAR sequences is described in International Application WO2014/153270 which is herein incorporated by reference in its entirety, including Examples 1-5 (p. 115-159).

In some embodiments, the CAR molecule is a humanized CD19 CAR comprising the amino acid sequence of:

(SEQ ID NO: 293)
EIVMTQSPATLSLSPGERATLSCRASQDISKYLNWYQQKPGQAPRLLIYH
TSRLHSGIPARFSGSGSGTDYTLTISSLQPEDFAVYFCQQGNTLPYTFGQ
GTKLEIKGGGGSGGGGSGGGGSQVQLQESGPGLVKPSETLSLTCTVSGVS
LPDYGVSWIRQPPGKGLEWIGVIWGSETTYYQSSLKSRVTISKDNSKNQV
SLKLSSVTAADTAVYYCAKHYYYGGSYAMDYWGQGTLVTVSS

In some embodiments, the CAR molecule is a humanized CD19 CAR comprising the amino acid sequence of:

(SEQ ID NO: 294)
EIVMTQSPATLSLSPGERATLSCRASQDISKYLNWYQQKPGQAPRLLIYH
TSRLHSGIPARFSGSGSGTDYTLTISSLQPEDFAVYFCQQGNTLPYTFGQ
GTKLEIKGGGGSGGGGSGGGGSQVQLQESGPGLVKPSETLSLTCTVSGVS
LPDYGVSWIRQPPGKGLEWIGVIWGSETTYYQSSLKSRVTISKDNSKNQV
SLKLSSVTAADTAVYYCAKHYYYGGSYAMDYWGQGTLVTVSSTTTPAPRP
PTPAPTIASQPLSLRPEACRPAAGGAVHTRGLDFACDIYIWAPLAGTCGV
LLLSLVITLYCKRGRKKLLYIFKQPFMRPVQTTQEEDGCSCRFPEEEEGG
CELRVKFSRSADAPAYKQGQNQLYNELNLGRREEYDVLDKRRGRDPEMGG
KPRRKNPQEGLYNELQKDKMAEAYSEIGMKGERRRGKGHDGLYQGLSTAT
KDTYDALHMQALPPR

In some embodiments, the CAR molecule is a humanized CD19 CAR comprising the amino acid sequence of:

(SEQ ID NO: 400)
MALPVTALLLPLALLLHAARPEIVMTQSPATLSLSPGERATLSCRASQD
ISKYLNWYQQKPGQAPRLLIYHTSRLHSGIPARFSGSGSGTDYTLTISS
LQPEDFAVYFCQQGNTLPYTFGQGTKLEIKGGGGSGGGGSGGGGSQVQL
QESGPGLVKPSETLSLTCTVSGVSLPDYGVSWIRQPPGKGLEWIGVIWG
SETTYYQSSLKSRVTISKDNSKNQVSLKLSSVTAADTAVYYCAKHYYYG
GSYAMDYWGQGTLVTVSSTTTPAPRPPTPAPTIASQPLSLRPEACRPAA
GGAVHTRGLDFACDIYIWAPLAGTCGVLLLSLVITLYCKRGRKKLLYIF
KQPFMRPVQTTQEEDGCSCRFPEEEEGGCELRVKFSRSADAPAYQQGQN
QLYNELNLGRREEYDVLDKRRGRDPEMGGKPRRKNPQEGLYNELQKDKM
AEAYSEIGMKGERRRGKGHDGLYQGLSTATKDTYDALHMQALPPR

In some embodiments, the CAR molecule is a humanized CD19 CAR comprising the amino acid sequence of:

(SEQ ID NO: 401)
EIVMTQSPATLSLSPGERATLSCRASQDISKYLNWYQQKPGQAPRLLIYH
TSRLHSGIPARFSGSGSGTDYTLTISSLQPEDFAVYFCQQGNTLPYTFGQ
GTKLEIKGGGGSGGGGSGGGGSQVQLQESGPGLVKPSETLSLTCTVSGVS
LPDYGVSWIRQPPGKGLEWIGVIWGSETTYYQSSLKSRVTISKDNSKNQV
SLKLSSVTAADTAVYYCAKHYYYGGSYAMDYWGQGTLVTVSSTTTPAPRP
PTPAPTIASQPLSLRPEACRPAAGGAVHTRGLDFACDIYIWAPLAGTCGV
LLLSLVITLYCKRGRKKLLYIFKQPFMRPVQTTQEEDGCSCRFPEEEEGG
CELRVKFSRSADAPAYQQGQNQLYNELNLGRREEYDVLDKRRGRDPEMGG
KPRRKNPQEGLYNELQKDKMAEAYSEIGMKGERRRGKGHDGLYQGLSTAT
KDTYDALHMQALPPR

Any known CD19 CAR, for example, the CD19 antigen binding domain of any known CD19 CAR, in the art can be used in accordance with the present disclosure. For example, LG-740; CD19 CAR described in the U.S. Pat. Nos. 8,399,645; 7,446,190; Xu et al., Leuk Lymphoma. 2013 54(2):255-260(2012); Cruz et al., Blood 122(17):2965-2973 (2013); Brentjens et al., Blood, 118(18):4817-4828 (2011); Kochenderfer et al., Blood 116(20):4099-102 (2010); Kochenderfer et al., Blood 122 (25):4129-39(2013); and 16th Annu Meet Am Soc Gen Cell Ther (ASGCT) (May 15-18, Salt Lake City) 2013, Abst 10.

Exemplary CD19 CARs include CD19 CARs described herein or an anti-CD19 CAR described in Xu et al. Blood 123.24(2014):3750-9; Kochenderfer et al. Blood 122.25(2013):4129-39, Cruz et al. Blood 122.17(2013):2965-73, NCT00586391, NCT01087294, NCT02456350, NCT00840853, NCT02659943, NCT02650999, NCT02640209, NCT01747486, NCT02546739, NCT02656147, NCT02772198, NCT00709033, NCT02081937, NCT00924326, NCT02735083, NCT02794246, NCT02746952, NCT01593696, NCT02134262, NCT01853631, NCT02443831, NCT02277522, NCT02348216, NCT02614066, NCT02030834, NCT02624258, NCT02625480, NCT02030847, NCT02644655, NCT02349698, NCT02813837, NCT02050347, NCT01683279, NCT02529813, NCT02537977, NCT02799550, NCT02672501, NCT02819583, NCT02028455, NCT01840566, NCT01318317, NCT01864889, NCT02706405, NCT01475058, NCT01430390, NCT02146924, NCT02051257, NCT02431988, NCT01815749, NCT02153580, NCT01865617, NCT02208362, NCT02685670, NCT02535364, NCT02631044, NCT02728882, NCT02735291, NCT01860937, NCT02822326, NCT02737085, NCT02465983, NCT02132624, NCT02782351, NCT01493453, NCT02652910, NCT02247609, NCT01029366, NCT01626495, NCT02721407, NCT01044069, NCT00422383, NCT01680991, NCT02794961, or NCT02456207, each of which is incorporated herein by reference in its entirety.

In some embodiments, CD19 CARs comprise a sequence, for example, a CDR, VH, VL, scFv, or full-CAR sequence, disclosed in Table 2, or a sequence having at least 80%, 85%, 90%, 95%, or 99% identity thereto.

TABLE 2
Amino acid sequences of exemplary anti-CD19 molecules

| SEQ ID NO | Region | Sequence |
|---|---|---|
| CTL019 | | |
| 295 | HCDR1 | DYGVS |
| | (Kabat) | |
| 296 | HCDR2 | VIWGSETTYYNSALKS |
| | (Kabat) | |
| 297 | HCDR3 | HYYYGGSYAMDY |
| | (Kabat) | |
| 298 | LCDR1 | RASQDISKYLN |
| | (Kabat) | |
| 299 | LCDR2 | HTSRLHS |
| | (Kabat) | |
| 300 | LCDR3 | QQGNTLPYT |
| | (Kabat) | |
| 301 | CTL019 | MALPVTALLLPLALLLHAARPDIQMTQTTSSLSASLGDRVTISCR |
| | Full amino | ASQDISKYLNWYQQKPDGTVKLLIYHTSRLHSGVPSRFSGSGSG |
| | acid | TDYSLTISNLEQEDIATYFCQQGNTLPYTFGGGTKLEITGGGGSG |
| | sequence | GGGSGGGGSEVKLQESGPGLVAPSQSLSVTCTVSGVSLPDYGVS |
| | | WIRQPPRKGLEWLGVIWGSETTYYNSALKSRLTIIKDNSKSQVF |
| | | LKMNSLQTDDTAIYYCAKHYYYGGSYAMDYWGQGTSVTVSST |
| | | TTPAPRPPTPAPTIASQPLSLRPEACRPAAGGAVHTRGLDFACDI |
| | | YIWAPLAGTCGVLLLSLVITLYCKRGRKKLLYIFKQPFMRPVQT |
| | | TQEEDGCSCRFPEEEEGGCELRVKFSRSADAPAYKQGQNQLYN |
| | | ELNLGRREEYDVLDKRRGRDPEMGGKPRRKNPQEGLYNELQK |
| | | DKMAEAYSEIGMKGERRRGKGHDGLYQGLSTATKDTYDALH |
| | | MQALPPR |
| 302 | CTL019 | ATGGCCTTACCAGTGACCGCCTTGCTCCTGCCGCTGGCCTTG |
| | Full | CTGCTCCACGCCGCCAGGCCGGACATCCAGATGACACAGACT |
| | nucleotide | ACATCCTCCCTGTCTGCCTCTCTGGGAGACAGAGTCACCATC |
| | sequence | AGTTGCAGGGCAAGTCAGGACATTAGTAAATATTTAAATTGG |
| | | TATCAGCAGAAACCAGATGGAACTGTTAAACTCCTGATCTAC |
| | | CATACATCAAGATTACACTCAGGAGTCCCATCAAGGTTCAGT |
| | | GGCAGTGGGTCTGGAACAGATTATTCTCTCACCATTAGCAAC |
| | | CTGGAGCAAGAAGATATTGCCACTTACTTTTGCCAACAGGGT |
| | | AATACGCTTCCGTACACGTTCGGAGGGGGGACCAAGCTGGA |
| | | GATCACAGGTGGCGGTGGCTCGGGCGGTGGTGGGTCGGGTG |
| | | GCGGCGGATCTGAGGTGAAACTGCAGGAGTCAGGACCTGGC |
| | | CTGGTGGCGCCCTCACAGAGCCTGTCCGTCACATGCACTGTC |
| | | TCAGGGGTCTCATTACCCGACTATGGTGTAAGCTGGATTCGC |
| | | CAGCCTCCACGAAAGGGTCTGGAGTGGCTGGGAGTAATATG |
| | | GGGTAGTGAAACCACATACTATAATTCAGCTCTCAAATCCAG |
| | | ACTGACCATCATCAAGGACAACTCCAAGAGCCAAGTTTTCTT |
| | | AAAAATGAACAGTCTGCAAACTGATGACACAGCCATTTACTA |
| | | CTGTGCCAAACATTATTACTACGGTGGTAGCTATGCTATGGA |
| | | CTACTGGGGCCAAGGAACCTCAGTCACCGTCTCCTCAACCAC |
| | | GACGCCAGCGCCGCGACCACCAACACCGGCGCCCACCATCG |
| | | CGTCGCAGCCCCTGTCCCTGCGCCCAGAGGCGTGCCGGCCAG |
| | | CGGCGGGGGGCGCAGTGCACACGAGGGGGCTGGACTTCGCC |
| | | TGTGATATCTACATCTGGGCGCCCTTGGCCGGGACTTGTGGG |
| | | GTCCTTCTCCTGTCACTGGTTATCACCCTTTACTGCAAACGGG |
| | | GCAGAAAGAAACTCCTGTATATATTCAAACAACCATTTATGA |
| | | GACCAGTACAAACTACTCAAGAGGAAGATGGCTGTAGCTGC |
| | | CGATTTCCAGAAGAAGAAGAAGGAGGATGTGAACTGAGAGT |
| | | GAAGTTCAGCAGGAGCGCAGACGCCCCCGCGTACAAGCAGG |
| | | GCCAGAACCAGCTCTATAACGAGCTCAATCTAGGACGAAGA |
| | | GAGGAGTACGATGTTTTGGACAAGAGACGTGGCCGGGACCC |
| | | TGAGATGGGGGGAAAGCCGAGAAGGAAGAACCCTCAGGAA |
| | | GGCCTGTACAATGAACTGCAGAAAGATAAGATGGCGGAGGC |
| | | CTACAGTGAGATTGGGATGAAAGGCGAGCGCCGGAGGGGCA |
| | | AGGGGCACGATGGCCTTTACCAGGGTCTCAGTACAGCCACCA |
| | | AGGACACCTACGACGCCCTTCACATGCAGGCCCTGCCCCCTC |
| | | GC |
| 303 | CTL019 | DIQMTQTTSSLSASLGDRVTISCRASQDISKYLNWYQQKPDGTV |
| | scFv domain | KLLIYHTSRLHSGVPSRFSGSGSGTDYSLTISNLEQEDIATYFCQQ |
| | | GNTLPYTFGGGTKLEITGGGGSGGGGSGGGGSEVKLQESGPGL |
| | | VAPSQSLSVTCTVSGVSLPDYGVSWIRQPPRKGLEWLGVIWGSE |
| | | TTYYNSALKSRLTIIKDNSKSQVFLKMNSLQTDDTAIYYCAKHY |
| | | YYGGSYAMDYWGQGTSVTVSS |
| Humanized | | |
| CAR2 | | |
| 295 | HCDR1 | DYGVS |
| | (Kabat) | |
| 304 | HCDR2 | VIWGSETTYYQSSLKS |
| | (Kabat) | |
| 297 | HCDR3 | HYYYGGSYAMDY |
| | (Kabat) | |
| 298 | LCDR1 | RASQDISKYLN |
| | (Kabat) | |
| 299 | LCDR2 | HTSRLHS |
| | (Kabat) | |
| 300 | LCDR3 | QQGNTLPYT |
| | (Kabat) | |
| 293 | CAR2 scFv | EIVMTQSPATLSLSPGERATLSCRASQDISKYLNWYQQKPGQAP |
| | domain-aa | RLLIYHTSRLHSGIPARFSGSGSGTDYTLTISSLQPEDFAVYFCQQ |
| | (Linker is | GNTLPYTFGQGTKLEIKGGGGSGGGGSGGGGSQVQLQESGPGL |
| | underlined) | VKPSETLSLTCTVSGVSLPDYGVSWIRQPPGKGLEWIGVIWGSE |
| | | TTYYQSSLKSRVTISKDNSKNQVSLKLSSVTAADTAVYYCAKH |
| | | YYYGGSYAMDYWGQGTLVTVSS |
| 305 | CAR2 scFv | atggccctccctgtcaccgccctgctgcttccgctggctcttctgctccacgccgctcggcccgaaatt |
| | domain-nt | gtgatgacccagtcacccgccactcttagcctttcacccggtgagcgcgcaaccctgtcttgcagagc |
| | | ctcccaagacatctcaaaataccttaattggtatcaacagaagcccggacaggctcctcgccttctgat |
| | | ctaccacaccagccggctccattctggaatccctgccaggttcagcggtagcggatctgggaccgac |
| | | tacaccctcactatcagctcactgcagccagaggacttcgctgtctatttctgtcagcaagggaacacc |
| | | ctgccctacacctttggacagggcaccaagctcgagattaaaggtggaggtggcagcggaggaggt |
| | | gggtccggcggtggaggaagccaggtccaactccaagaaagcggaccgggtcttgtgaagccatc |
| | | agaaactctttcactgacttgtactgtgagcggagtgtctctccccgattacggggtgtcttggatcaga |
| | | cagccaccggggaagggtctggaatggattggagtgatttggggctctgagactacttactaccaatc |
| | | atccctcaagtcacgcgtcaccatctcaaaggacaactctaagaatcaggtgtcactgaaactgtcatc |
| | | tgtgaccgcagccgacaccgccgtgtactattgcgctaagcattactattatggcgggagctacgcaa |
| | | tggattactggggacagggtactctggtcaccgtgtccagccaccaccatcatcaccatcaccat |
| 306 | CAR 2- | MALPVTALLLPLALLLHAARPEIVMTQSPATLSLSPGERATLSCR |
| | Full-aa | ASQDISKYLNWYQQKPGQAPRLLIYHTSRLHSGIPARFSGSGSG |
| | | TDYTLTISSLQPEDFAVYFCQQGNTLPYTFGQGTKLEIKGGGGS |
| | | GGGGSGGGGSQVQLQESGPGLVKPSETLSLTCTVSGVSLPDYG |
| | | VSWIRQPPGKGLEWIGVIWGSETTYYQSSLKSRVTISKDNSKNQ |
| | | VSLKLSSVTAADTAVYYCAKHYYYGGSYAMDYWGQGTLVTV |
| | | SSTTTPAPRPPTPAPTIASQPLSLRPEACRPAAGGAVHTRGLDFA |
| | | CDIYIWAPLAGTCGVLLLSLVITLYCKRGRKKLLYIFKQPFMRP |
| | | VQTTQEEDGCSCRFPEEEEGGCELRVKFSRSADAPAYKQGQNQ |
| | | LYNELNLGRREEYDVLDKRRGRDPEMGGKPRRKNPQEGLYNE |
| | | LQKDKMAEAYSEIGMKGERRRGKGHDGLYQGLSTATKDTYDA |
| | | LHMQALPPR |
| 307 | CAR 2- | atggccctccctgtcaccgccctgctgcttccgctggctcttctgctccacgccgctcggcccgaaatt |
| | Full-nt | gtgatgacccagtcacccgccactcttagcctttcacccggtgagcgcgcaaccctgtcttgcagagc |
| | | ctcccaagacatctcaaaataccttaattggtatcaacagaagcccggacaggctcctcgccttctgat |
| | | ctaccacaccagccggctccattctggaatccctgccaggttcagcggtagcggatctgggaccgac |
| | | tacaccctcactatcagctcactgcagccagaggacttcgctgtctatttctgtcagcaagggaacacc |
| | | ctgccctacacctttggacagggcaccaagctcgagattaaaggtggaggtggcagcggaggaggt |
| | | gggtccggcggtggaggaagccaggtccaactccaagaaagcggaccgggtcttgtgaagccatc |
| | | agaaactctttcactgacttgtactgtgagcggagtgtctctccccgattacggggtgtcttggatcaga |
| | | cagccaccggggaagggtctggaatggattggagtgatttggggctctgagactacttactaccaatc |
| | | atccctcaagtcacgcgtcaccatctcaaaggacaactctaagaatcaggtgtcactgaaactgtcatc |
| | | tgtgaccgcagccgacaccgccgtgtactattgcgctaagcattactattatggcgggagctacgcaa |
| | | tggattactggggacagggtactctggtcaccgtgtccagcaccactaccccagcaccgaggccac |
| | | ccaccccggctcctaccatcgcctcccagcctctgtccctgcgtccggaggcatgtagacccgcagc |
| | | tggtggggccgtgcatacccggggtcttgacttcgcctgcgatatctacatttgggcccctctggctgg |
| | | tacttgcggggtcctgctgctttcactcgtgatcactctttactgtaagcgcggtcggaagaagctgctg |
| | | tacatctttaagcaacccttcatgaggcctgtgcagactactcaagaggaggacggctgttcatgccg |
| | | gttcccagaggaggaggaaggcggctgcgaactgcgcgtgaaattcagccgcagcgcagatgctc |
| | | cagcctacaagcaggggcagaaccagctctacaacgaactcaatcttggtcggagagaggagtacg |
| | | acgtgctggacaagcggagaggacgggacccagaaatgggcgggaagccgcgcagaaagaatc |
| | | cccaagagggcctgtacaacgagctccaaaaggataagatggcagaagcctatagcgagattggta |
| | | tgaaaggggaacgcagaagaggcaaaggccacgacggactgtaccagggactcagcaccgcca |
| | | ccaaggacacctatgacgctcttcacatgcaggccctgccgcctcgg |
| 397 | CAR 2- | gaaattgtgatgacccagtcacccgccactcttagcctttcacccggtgagcgcgcaaccctgtcttgc |
| | Full-nt; no | agagcctcccaagacatctcaaaataccttaattggtatcaacagaagcccggacaggctcctcgcct |
| | leader | tctgatctaccacaccagccggctccattctggaatccctgccaggttcagcggtagcggatctggga |
| | | ccgactacaccctcactatcagctcactgcagccagaggacttcgctgtctatttctgtcagcaaggga |
| | | acaccctgccctacacctttggacagggcaccaagctcgagattaaaggtggaggtggcagcggag |
| | | gaggtgggtccggcggtggaggaagccaggtccaactccaagaaagcggaccgggtcttgtgaag |
| | | ccatcagaaactctttcactgacttgtactgtgagcggagtgtctctccccgattacggggtgtcttggat |
| | | cagacagccaccggggaagggtctggaatggattggagtgatttggggctctgagactacttactac |
| | | caatcatccctcaagtcacgcgtcaccatctcaaaggacaactctaagaatcaggtgtcactgaaact |
| | | gtcatctgtgaccgcagccgacaccgccgtgtactattgcgctaagcattactattatggcgggagcta |
| | | cgcaatggattactggggacagggtactctggtcaccgtgtccagcaccactaccccagcaccgag |
| | | gccacccaccccggctcctaccatcgcctcccagcctctgtccctgcgtccggaggcatgtagaccc |
| | | gcagctggtggggccgtgcatacccggggtcttgacttcgcctgcgatatctacatttgggcccctctg |
| | | gctggtacttgcggggtcctgctgctttcactcgtgatcactctttactgtaagcgcggtcggaagaag |
| | | ctgctgtacatctttaagcaacccttcatgaggcctgtgcagactactcaagaggaggacggctgttca |
| | | tgccggttcccagaggaggaggaaggcggctgcgaactgcgcgtgaaattcagccgcagcgcag |
| | | atgctccagcctacaagcaggggcagaaccagctctacaacgaactcaatcttggtcggagagagg |
| | | agtacgacgtgctggacaagcggagaggacgggacccagaaatgggcgggaagccgcgcagaa |
| | | agaatccccaagagggcctgtacaacgagctccaaaaggataagatggcagaagcctatagcgag |
| | | attggtatgaaaggggaacgcagaagaggcaaaggccacgacggactgtaccagggactcagca |
| | | ccgccaccaaggacacctatgacgctcttcacatgcaggccctgccgcctcgg |
| 398 | CAR 2- | atggccctccctgtcaccgccctgctgcttccgctggctcttctgctccacg |
| | Full-nt | ccgctcggcccgaaattgtgatgacccagtcacccgccactcttagcctttc |
| | with hairpin | acccggtgagcgcgcaaccctgtcttgcagagcctcccaagacatctcaaaa |
| | modification | taccttaattggtatcaacagaagcccggacaggctcctcgccttctgatct |
| | | accacaccagccggctccattctggaatccctgccaggttcagcggtagcgg |
| | | atctgggaccgactacaccctcactatcagctcactgcagccagaggacttc |
| | | gctgtctatttctgtcagcaagggaacaccctgccctacacctttggacagg |
| | | gcaccaagctcgagattaaaggtggaggtggcagcggaggaggtgggtccgg |
| | | cggtggaggaagccaggtccaactccaagaaagcggaccgggtcttgtgaag |
| | | ccatcagaaactctttcactgacttgtactgtgagcggagtgtctctccccg |
| | | attacggggtgtcttggatcagacagccaccggggaagggtctggaatggat |
| | | tggagtgatttggggctcAgagactacttactaccaatcatccctcaagtcT |
| | | cgcgtcaccatctcaaaggacaactctaagaatcaggtgtcactgaaactgt |
| | | catctgtgaccgcagccgacaccgccgtgtactattgcgctaagcattacta |
| | | ttatggcgggagctacgcaatggattactggggacagggtactctggtcacc |
| | | gtgtccagcaccactaccccagcaccgaggccacccaccccggctcctacca |
| | | tcgcctcccagcctctgtccctgcgtccggaggcatgtagacccgcagctgg |
| | | tggggccgtgcatacccggggtcttgacttcgcctgcgatatctacatttgg |
| | | gcccctctggctggtacttgcggggtcctgctgctttcactcgtgatcactc |
| | | tttactgtaagcgcggtcggaagaagctgctgtacatctttaagcaaccctt |
| | | catgaggcctgtgcagactactcaagaggaggacggctgttcatgccggttc |
| | | ccagaggaggaggaaggcggctgcgaactgcgcgtgaaattcagccgcagcg |
| | | cagatgctccagcctacAagcaggggcagaaccagctctacaacgaactcaa |
| | | tcttggtcggagagaggagtacgacgtgctggacaagcggagaggacgggac |
| | | ccagaaatgggcgggaagccgcgcagaaagaatccccaagagggcctgtaca |
| | | acgagctccaaaaggataagatggcagaagcctatagcgagattggtatgaa |
| | | aggggaacgcagaagaggcaaaggccacgacggactgtaccagggactcagc |
| | | accgccaccaaggacacctatgacgctcttcacatgcaggccctgccgcctc |
| | | gg |
| 399 | CAR 2- | gaaattgtgatgacccagtcacccgccactcttagcctttcacccggtgagc |
| | Full-nt | gcgcaaccctgtcttgcagagcctcccaagacatctcaaaataccttaattg |
| | with hairpin | gtatcaacagaagcccggacaggctcctcgccttctgatctaccacaccagc |
| | modification | cggctccattctggaatccctgccaggttcagcggtagcggatctgggaccg |
| | and no | actacaccctcactatcagctcactgcagccagaggacttcgctgtctattt |
| | leader | ctgtcagcaagggaacaccctgccctacacctttggacagggcaccaagctc |
| | | gagattaaaggtggaggtggcagcggaggaggtgggtccggcggtggaggaa |
| | | gccaggtccaactccaagaaagcggaccgggtcttgtgaagccatcagaaac |
| | | tctttcactgacttgtactgtgagcggagtgtctctccccgattacggggtg |
| | | tcttggatcagacagccaccggggaagggtctggaatggattggagtgattt |
| | | ggggctcAgagactacttactaccaatcatccctcaagtcTcgcgtcaccat |
| | | ctcaaaggacaactctaagaatcaggtgtcactgaaactgtcatctgtgacc |
| | | gcagccgacaccgccgtgtactattgcgctaagcattactattatggcggga |
| | | gctacgcaatggattactggggacagggtactctggtcaccgtgtccagcac |
| | | cactaccccagcaccgaggccacccaccccggctcctaccatcgcctcccag |
| | | cctctgtccctgcgtccggaggcatgtagacccgcagctggtggggccgtgc |
| | | atacccggggtcttgacttcgcctgcgatatctacatttgggcccctctggc |
| | | tggtacttgcggggtcctgctgctttcactcgtgatcactctttactgtaag |
| | | cgcggtcggaagaagctgctgtacatctttaagcaacccttcatgaggcctg |
| | | tgcagactactcaagaggaggacggctgttcatgccggttcccagaggagga |
| | | ggaaggcggctgcgaactgcgcgtgaaattcagccgcagcgcagatgctcca |
| | | gcctacAagcaggggcagaaccagctctacaacgaactcaatcttggtcgga |
| | | gagaggagtacgacgtgctggacaagcggagaggacgggacccagaaatggg |
| | | cgggaagccgcgcagaaagaatccccaagagggcctgtacaacgagctccaa |
| | | aaggataagatggcagaagcctatagcgagattggtatgaaaggggaacgca |
| | | gaagaggcaaaggccacgacggactgtaccagggactcagcaccgccaccaa |
| | | ggacacctatgacgctcttcacatgcaggccctgccgcctcgg |
| 400 | CAR2A- | MALPVTALLLPLALLLHAARPEIVMTQSPATLSLSPGERATLSCRASQDISK |
| | leader | YLNWYQQKPGQAPRLLIYHTSRLHSGIPARFSGSGSGTDYTLTISSLQPEDF |
| | underlined | AVYFCQQGNTLPYTFGQGTKLEIKGGGGSGGGGSGGGGSQVQLQESGPGLVK |
| | Full-aa; | PSETLSLTCTVSGVSLPDYGVSWIRQPPGKGLEWIGVIWGSETTYYQSSLKS |
| | | RVTISKDNSKNQVSLKLSSVTAADTAVYYCAKHYYYGGSYAMDYWGQGTLVT |
| | | VSSTTTPAPRPPTPAPTIASQPLSLRPEACRPAAGGAVHTRGLDFACDIYIW |
| | | APLAGTCGVLLLSLVITLYCKRGRKKLLYIFKQPFMRPVQTTQEEDGCSCRF |
| | | PEEEEGGCELRVKFSRSADAPAYQQGQNQLYNELNLGRREEYDVLDKRRGRD |
| | | PEMGGKPRRKNPQEGLYNELQKDKMAEAYSEIGMKGERRRGKGHDGLYQGLS |
| | | TATKDTYDALHMQALPPR |
| 401 | CAR2A- | EIVMTQSPATLSLSPGERATLSCRASQDISKYLNWYQQKPGQAPRLLIYHTS |
| | Full-aa; no | RLHSGIPARFSGSGSGTDYTLTISSLQPEDFAVYFCQQGNTLPYTFGQGTKL |
| | leader | EIKGGGGSGGGGSGGGGSQVQLQESGPGLVKPSETLSLTCTVSGVSLPDYGV |
| | | SWIRQPPGKGLEWIGVIWGSETTYYQSSLKSRVTISKDNSKNQVSLKLSSVT |
| | | AADTAVYYCAKHYYYGGSYAMDYWGQGTLVTVSSTTTPAPRPPTPAPTIASQ |
| | | PLSLRPEACRPAAGGAVHTRGLDFACDIYIWAPLAGTCGVLLLSLVITLYCK |
| | | RGRKKLLYIFKQPFMRPVQTTQEEDGCSCRFPEEEEGGCELRVKFSRSADAP |
| | | AYQQGQNQLYNELNLGRREEYDVLDKRRGRDPEMGGKPRRKNPQEGLYNELQ |
| | | KDKMAEAYSEIGMKGERRRGKGHDGLYQGLSTATKDTYDALHMQALPPR |
| 402 | CAR2A- | atccccctccctctcacccccctcctccttcccctccctcttctcctccacccccctccccccgaaatt |
| | leader | gtgatgacccagtcacccgccactcttagcctttcacccggtgagcgcgcaaccctgtcttgcagagc |
| | underlined | ctcccaagacatctcaaaataccttaattggtatcaacagaagcccggacaggctcctcgccttctgat |
| | Full-nt; | ctaccacaccagccggctccattctggaatccctgccaggttcagcggtagcggatctgggaccgac |
| | | tacaccctcactatcagctcactgcagccagaggacttcgctgtctatttctgtcagcaagggaacacc |
| | | ctgccctacacctttggacagggcaccaagctcgagattaaaggtggaggtggcagcggaggaggt |
| | | gggtccggcggtggaggaagccaggtccaactccaagaaagcggaccgggtcttgtgaagccatc |
| | | agaaactctttcactgacttgtactgtgagcggagtgtctctccccgattacggggtgtcttggatcaga |
| | | cagccaccggggaagggtctggaatggattggagtgatttggggctctgagactacttactaccaatc |
| | | atccctcaagtcacgcgtcaccatctcaaaggacaactctaagaatcaggtgtcactgaaactgtcatc |
| | | tgtgaccgcagccgacaccgccgtgtactattgcgctaagcattactattatggcgggagctacgcaa |
| | | tggattactggggacagggtactctggtcaccgtgtccagcaccactaccccagcaccgaggccac |
| | | ccaccccggctcctaccatcgcctcccagcctctgtccctgcgtccggaggcatgtagacccgcagc |
| | | tggtggggccgtgcatacccggggtcttgacttcgcctgcgatatctacatttgggcccctctggctgg |
| | | tacttgcggggtcctgctgctttcactcgtgatcactctttactgtaagcgcggtcggaagaagctgctg |
| | | tacatctttaagcaacccttcatgaggcctgtgcagactactcaagaggaggacggctgttcatgccg |
| | | gttcccagaggaggaggaaggcggctgcgaactgcgcgtgaaattcagccgcagcgcagatgctc |
| | | cagcctaccagcaggggcagaaccagctctacaacgaactcaatcttggtcggagagaggagtacg |
| | | acgtgctggacaagcggagaggacgggacccagaaatgggcgggaagccgcgcagaaagaatc |
| | | cccaagagggcctgtacaacgagctccaaaaggataagatggcagaagcctatagcgagattggta |
| | | tgaaaggggaacgcagaagaggcaaaggccacgacggactgtaccagggactcagcaccgcca |
| | | ccaaggacacctatgacgctcttcacatgcaggccctgccgcctcgg |
| 403 | CAR2A- | gaaattgtgatgacccagtcacccgccactcttagcctttcacccggtgagcgcgcaaccctgtcttgc |
| | Full-nt; no | agagcctcccaagacatctcaaaataccttaattggtatcaacagaagcccggacaggctcctcgcct |
| | leader | tctgatctaccacaccagccggctccattctggaatccctgccaggttcagcggtagcggatctggga |
| | | ccgactacaccctcactatcagctcactgcagccagaggacttcgctgtctatttctgtcagcaaggga |
| | | acaccctgccctacacctttggacagggcaccaagctcgagattaaaggtggaggtggcagcggag |
| | | gaggtgggtccggcggtggaggaagccaggtccaactccaagaaagcggaccgggtcttgtgaag |
| | | ccatcagaaactctttcactgacttgtactgtgagcggagtgtctctccccgattacggggtgtcttggat |
| | | cagacagccaccggggaagggtctggaatggattggagtgatttggggctctgagactacttactac |
| | | caatcatccctcaagtcacgcgtcaccatctcaaaggacaactctaagaatcaggtgtcactgaaact |
| | | gtcatctgtgaccgcagccgacaccgccgtgtactattgcgctaagcattactattatggcgggagcta |
| | | cgcaatggattactggggacagggtactctggtcaccgtgtccagcaccactaccccagcaccgag |
| | | gccacccaccccggctcctaccatcgcctcccagcctctgtccctgcgtccggaggcatgtagaccc |
| | | gcagctggtggggccgtgcatacccggggtcttgacttcgcctgcgatatctacatttgggcccctctg |
| | | gctggtacttgcggggtcctgctgctttcactcgtgatcactctttactgtaagcgcggtcggaagaag |
| | | ctgctgtacatctttaagcaacccttcatgaggcctgtgcagactactcaagaggaggacggctgttca |
| | | tgccggttcccagaggaggaggaaggcggctgcgaactgcgcgtgaaattcagccgcagcgcag |
| | | atgctccagcctaccagcaggggcagaaccagctctacaacgaactcaatcttggtcggagagagg |
| | | agtacgacgtgctggacaagcggagaggacgggacccagaaatgggcgggaagccgcgcagaa |
| | | agaatccccaagagggcctgtacaacgagctccaaaaggataagatggcagaagcctatagcgag |
| | | attggtatgaaaggggaacgcagaagaggcaaaggccacgacggactgtaccagggactcagca |
| | | ccgccaccaaggacacctatgacgctcttcacatgcaggccctgccgcctcgg |
| 404 | CAR2A- | atggccctccctgtcaccgccctgctgcttccgctggctcttctgctccacg |
| | with hairpin | ccgctcggcccgaaattgtgatgacccagtcacccgccactcttagcctttc |
| | modification | acccggtgagcgcgcaaccctgtcttgcagagcctcccaagacatctcaaaa |
| | and leader | taccttaattggtatcaacagaagcccggacaggctcctcgccttctgatct |
| | underlined | accacaccagccggctccattctggaatccctgccaggttcagcggtagcgg |
| | Full-nt; | atctgggaccgactacaccctcactatcagctcactgcagccagaggacttc |
| | | gctgtctatttctgtcagcaagggaacaccctgccctacacctttggacagg |
| | | gcaccaagctcgagattaaaggtggaggtggcagcggaggaggtgggtccgg |
| | | cggtggaggaagccaggtccaactccaagaaagcggaccgggtcttgtgaag |
| | | ccatcagaaactctttcactgacttgtactgtgagcggagtgtctctccccg |
| | | attacggggtgtcttggatcagacagccaccggggaagggtctggaatggat |
| | | tggagtgatttggggctcAgagactacttactaccaatcatccctcaagtcT |
| | | cgcgtcaccatctcaaaggacaactctaagaatcaggtgtcactgaaactgt |
| | | catctgtgaccgcagccgacaccgccgtgtactattgcgctaagcattacta |
| | | ttatggcgggagctacgcaatggattactggggacagggtactctggtcacc |
| | | gtgtccagcaccactaccccagcaccgaggccacccaccccggctcctacca |
| | | tcgcctcccagcctctgtccctgcgtccggaggcatgtagacccgcagctgg |
| | | tggggccgtgcatacccggggtcttgacttcgcctgcgatatctacatttgg |
| | | gcccctctggctggtacttgcggggtcctgctgctttcactcgtgatcactc |
| | | tttactgtaagcgcggtcggaagaagctgctgtacatctttaagcaaccctt |
| | | catgaggcctgtgcagactactcaagaggaggacggctgttcatgccggttc |
| | | ccagaggaggaggaaggcggctgcgaactgcgcgtgaaattcagccgcagcg |
| | | cagatgctccagcctacCagcaggggcagaaccagctctacaacgaactcaa |
| | | tcttggtcggagagaggagtacgacgtgctggacaagcggagaggacgggac |
| | | ccagaaatgggcgggaagccgcgcagaaagaatccccaagagggcctgtaca |
| | | acgagctccaaaaggataagatggcagaagcctatagcgagattggtatgaa |
| | | aggggaacgcagaagaggcaaaggccacgacggactgtaccagggactcagc |
| | | accgccaccaaggacacctatgacgctcttcacatgcaggccctgccgcctc |
| | | gg |
| 405 | CAR2A- | gaaattgtgatgacccagtcacccgccactcttagcctttcacccggtgagc |
| | Full-nt; | gcgcaaccctgtcttgcagagcctcccaagacatctcaaaataccttaattg |
| | with hairpin | gtatcaacagaagcccggacaggctcctcgccttctgatctaccacaccagc |
| | modification | cggctccattctggaatccctgccaggttcagcggtagcggatctgggaccg |
| | and no | actacaccctcactatcagctcactgcagccagaggacttcgctgtctattt |
| | leader | ctgtcagcaagggaacaccctgccctacacctttggacagggcaccaagctc |
| | | gagattaaaggtggaggtggcagcggaggaggtgggtccggcggtggaggaa |
| | | gccaggtccaactccaagaaagcggaccgggtcttgtgaagccatcagaaac |
| | | tctttcactgacttgtactgtgagcggagtgtctctccccgattacggggtg |
| | | tcttggatcagacagccaccggggaagggtctggaatggattggagtgattt |
| | | ggggctcAgagactacttactaccaatcatccctcaagtcTcgcgtcaccat |
| | | ctcaaaggacaactctaagaatcaggtgtcactgaaactgtcatctgtgacc |
| | | gcagccgacaccgccgtgtactattgcgctaagcattactattatggcggga |
| | | gctacgcaatggattactggggacagggtactctggtcaccgtgtccagcac |
| | | cactaccccagcaccgaggccacccaccccggctcctaccatcgcctcccag |
| | | cctctgtccctgcgtccggaggcatgtagacccgcagctggtggggccgtgc |
| | | atacccggggtcttgacttcgcctgcgatatctacatttgggcccctctggc |
| | | tggtacttgcggggtcctgctgctttcactcgtgatcactctttactgtaag |
| | | cgcggtcggaagaagctgctgtacatctttaagcaacccttcatgaggcctg |
| | | tgcagactactcaagaggaggacggctgttcatgccggttcccagaggagga |
| | | ggaaggcggctgcgaactgcgcgtgaaattcagccgcagcgcagatgctcca |
| | | gcctacCagcaggggcagaaccagctctacaacgaactcaatcttggtcgga |
| | | gagaggagtacgacgtgctggacaagcggagaggacgggacccagaaatggg |
| | | cgggaagccgcgcagaaagaatccccaagagggcctgtacaacgagctccaa |
| | | aaggataagatggcagaagcctatagcgagattggtatgaaaggggaacgca |
| | | gaagaggcaaaggccacgacggactgtaccagggactcagcaccgccaccaa |
| | | ggacacctatgacgctcttcacatgcaggccctgccgcctcgg |
| CAR A | | |
| 406 | CARA- | atgcttctcctggtgacaagccttctgctctgtgagttaccacacccagcat |
| | with leader | tcctcctgatcccagacatccagatgacacagactacatcctccctgtctgc |
| | Full-nt; | ctctctgggagacagagtcaccatcagttgcagggcaagtcaggacattagt |
| | | aaatatttaaattggtatcagcagaaaccagatggaactgttaaactcctga |
| | | tctaccatacatcaagattacactcaggagtcccatcaaggttcagtggcag |
| | | tgggtctggaacagattattctctcaccattagcaacctggagcaagaagat |
| | | attgccacttacttttgccaacagggtaatacgcttccgtacacgttcggag |
| | | gggggactaagttggaaataacaggctccacctctggatccggcaagcccgg |
| | | atctggcgagggatccaccaagggcgaggtgaaactgcaggagtcaggacct |
| | | ggcctggtggcgccctcacagagcctgtccgtcacatgcactgtctcagggg |
| | | tctcattacccgactatggtgtaagctggattcgccagcctccacgaaaggg |
| | | tctggagtggctgggagtaatatggggtagtgaaaccacatactataattca |
| | | gctctcaaatccagactgaccatcatcaaggacaactccaagagccaagttt |
| | | tcttaaaaatgaacagtctgcaaactgatgacacagccatttactactgtgc |
| | | caaacattattactacggtggtagctatgctatggactactggggtcaagga |
| | | acctcagtcaccgtctcctcagcggccgcaattgaagttatgtatcctcctc |
| | | cttacctagacaatgagaagagcaatggaaccattatccatgtgaaagggaa |
| | | acacctttgtccaagtcccctatttcccggaccttctaagcccttttgggtg |
| | | ctggtggtggttgggggagtcctggcttgctatagcttgctagtaacagtgg |
| | | cctttattattttctgggtgaggagtaagaggagcaggctcctgcacagtga |
| | | ctacatgaacatgactccccgccgccccgggcccacccgcaagcattaccag |
| | | ccctatgccccaccacgcgacttcgcagcctatcgctccagagtgaagttca |
| | | gcaggagcgcagacgcccccgcgtaccagcagggccagaaccagctctataa |
| | | cgagctcaatctaggacgaagagaggagtacgatgttttggacaagagacgt |
| | | ggccgggaccctgagatggggggaaagccgagaaggaagaaccctcaggaag |
| | | gcctgtacaatgaactgcagaaagataagatggcggaggcctacagtgagat |
| | | tgggatgaaaggcgagcgccggaggggcaaggggcacgatggcctttaccag |
| | | ggtctcagtacagccaccaaggacacctacgacgcccttcacatgcaggccc |
| | | tgccccctcgc |
| 407 | CARA-full | MLLLVTSLLLCELPHPAFLLIPDIQMTQTTSSLSASLGDRVTISCRASQDIS |
| | amino acid | KYLNWYQQKPDGTVKLLIYHTSRLHSGVPSRFSGSGSGTDYSLTISNLEQED |
| | transgene | IATYFCQQGNTLPYTFGGGTKLEITGSTSGSGKPGSGEGSTKGEVKLQESGP |
| | sequence; | GLVAPSQSLSVTCTVSGVSLPDYGVSWIRQPPRKGLEWLGVIWGSETTYYNS |
| | with leader | ALKSRLTIIKDNSKSQVFLKMNSLQTDDTAIYYCAKHYYYGGSYAMDYWGQG |
| | underlined | TSVTVSSAAAIEVMYPPPYLDNEKSNGTIIHVKGKHLCPSPLFPGPSKPFWV |
| | | LVVVGGVLACYSLLVTVAFIIFWVRSKRSRLLHSDYMNMTPRRPGPTRKHYQ |
| | | PYAPPRDFAAYRSRVKFSRSADAPAYQQGQNQLYNELNLGRREEYDVLDKRR |
| | | GRDPEMGGKPRRKNPQEGLYNELQKDKMAEAYSEIGMKGERRRGKGHDGLYQ |
| | | GLSTATKDTYDALHMQALPPR |
| 408 | CARA- | atgcttctcctggtgacaagccttctgctctgtgagttaccacacccagcat |
| | nucleotide | tcctcctgatcccagacatccagatgacacagactacatcctccctgtctgc |
| | sequence | ctctctgggagacagagtcaccatcagttgcagggcaagtcaggacattagt |
| | with leader | aaatatttaaattggtatcagcagaaaccagatggaactgttaaactcctga |
| | underlined | tctaccatacatcaagattacactcaggagtcccatcaaggttcagtggcag |
| | CD19 scFv | tgggtctggaacagattattctctcaccattagcaacctggagcaagaagat |
| | | attgccacttacttttgccaacagggtaatacgcttccgtacacgttcggag |
| | | gggggactaagttggaaataacaggctccacctctggatccggcaagcccgg |
| | | atctggcgagggatccaccaagggcgaggtgaaactgcaggagtcaggacct |
| | | ggcctggtggcgccctcacagagcctgtccgtcacatgcactgtctcagggg |
| | | tctcattacccgactatggtgtaagctggattcgccagcctccacgaaaggg |
| | | tctggagtggctgggagtaatatggggtagtgaaaccacatactataattca |
| | | gctctcaaatccagactgaccatcatcaaggacaactccaagagccaagttt |
| | | tcttaaaaatgaacagtctgcaaactgatgacacagccatttactactgtgc |
| | | caaacattattactacggtggtagctatgctatggactactggggtcaagga |
| | | acctcagtcaccgtctcctca |
| 409 | CARA- | MLLLVTSLLLCELPHPAFLLIPDIQMTQTTSSLSASLGDRVTISCRASQDIS |
| | CD 19 scFv | KYLNWYQQKPDGTVKLLIYHTSRLHSGVPSRFSGSGSGTDYSLTISNLEQED |
| | amino acid | IATYFCQQGNTLPYTFGGGTKLEITGSTSGSGKPGSGEGSTKGEVKLQESGP |
| | sequence; | GLVAPSQSLSVTCTVSGVSLPDYGVSWIRQPPRKGLEWLGVIWGSETTYYNS |
| | with leader | ALKSRLTIIKDNSKSQVFLKMNSLQTDDTAIYYCAKHYYYGGSYAMDYWGQG |
| | underlined | TSVTVS |
| 410 | CARA-full | gacatccagatgacacagactacatcctccctgtctgcctctctgggagaca |
| | nucleotide | gagtcaccatcagttgcagggcaagtcaggacattagtaaatatttaaattg |
| | sequence; | gtatcagcagaaaccagatggaactgttaaactcctgatctaccatacatca |
| | no leader | agattacactcaggagtcccatcaaggttcagtggcagtgggtctggaacag |
| | | attattctctcaccattagcaacctggagcaagaagatattgccacttactt |
| | | ttgccaacagggtaatacgcttccgtacacgttcggaggggggactaagttg |
| | | gaaataacaggctccacctctggatccggcaagcccggatctggcgagggat |
| | | ccaccaagggcgaggtgaaactgcaggagtcaggacctggcctggtggcgcc |
| | | ctcacagagcctgtccgtcacatgcactgtctcaggggtctcattacccgac |
| | | tatggtgtaagctggattcgccagcctccacgaaagggtctggagtggctgg |
| | | gagtaatatggggtagtgaaaccacatactataattcagctctcaaatccag |
| | | actgaccatcatcaaggacaactccaagagccaagttttcttaaaaatgaac |
| | | agtctgcaaactgatgacacagccatttactactgtgccaaacattattact |
| | | acggtggtagctatgctatggactactggggtcaaggaacctcagtcaccgt |
| | | ctcctcagcggccgcaattgaagttatgtatcctcctccttacctagacaat |
| | | gagaagagcaatggaaccattatccatgtgaaagggaaacacctttgtccaa |
| | | gtcccctatttcccggaccttctaagcccttttgggtgctggtggtggttgg |
| | | gggagtcctggcttgctatagcttgctagtaacagtggcctttattattttc |
| | | tgggtgaggagtaagaggagcaggctcctgcacagtgactacatgaacatga |
| | | ctccccgccgccccgggcccacccgcaagcattaccagccctatgccccacc |
| | | acgcgacttcgcagcctatcgctccagagtgaagttcagcaggagcgcagac |
| | | gcccccgcgtaccagcagggccagaaccagctctataacgagctcaatctag |
| | | gacgaagagaggagtacgatgttttggacaagagacgtggccgggaccctga |
| | | gatggggggaaagccgagaaggaagaaccctcaggaaggcctgtacaatgaa |
| | | ctgcagaaagataagatggcggaggcctacagtgagattgggatgaaaggcg |
| | | agcgccggaggggcaaggggcacgatggcctttaccagggtctcagtacagc |
| | | caccaaggacacctacgacgcccttcacatgcaggccctgccccctcgc |
| 411 | CARA-full | DIQMTQTTSSLSASLGDRVTISCRASQDISKYLNWYQQKPDGTVKLLIYHTS |
| | amino acid | RLHSGVPSRFSGSGSGTDYSLTISNLEQEDIATYFCQQGNTLPYTFGGGTKL |
| | transgene | EITGSTSGSGKPGSGEGSTKGEVKLQESGPGLVAPSQSLSVTCTVSGVSLPD |
| | sequence; | YGVSWIRQPPRKGLEWLGVIWGSETTYYNSALKSRLTIIKDNSKSQVFLKMN |
| | no leader | SLQTDDTAIYYCAKHYYYGGSYAMDYWGQGTSVTVSSAAAIEVMYPPPYLDN |
| | | EKSNGTIIHVKGKHLCPSPLFPGPSKPFWVLVWGGVLACYSLLVTVAFIIF |
| | | WVRSKRSRLLHSDYMNMTPRRPGPTRKHYQPYAPPRDFAAYRSRVKFSRSAD |
| | | APAYQQGQNQLYNELNLGRREEYDVLDKRRGRDPEMGGKPRRKNPQEGLYNE |
| | | LQKDKMAEAYSEIGMKGERRRGKGHDGLYQGLSTATKDTYDALHMQALPPR |
| 412 | CARA- | gacatccagatgacacagactacatcctccctgtctgcctctctgggagaca |
| | CD 19 scFv | gagtcaccatcagttgcagggcaagtcaggacattagtaaatatttaaattg |
| | nucleotide; | gtatcagcagaaaccagatggaactgttaaactcctgatctaccatacatca |
| | no leader | agattacactcaggagtcccatcaaggttcagtggcagtgggtctggaacag |
| | | attattctctcaccattagcaacctggagcaagaagatattgccacttactt |
| | | ttgccaacagggtaatacgcttccgtacacgttcggaggggggactaagttg |
| | | gaaataacaggctccacctctggatccggcaagcccggatctggcgagggat |
| | | ccaccaagggcgaggtgaaactgcaggagtcaggacctggcctggtggcgcc |
| | | ctcacagagcctgtccgtcacatgcactgtctcaggggtctcattacccgac |
| | | tatggtgtaagctggattcgccagcctccacgaaagggtctggagtggctgg |
| | | gagtaatatggggtagtgaaaccacatactataattcagctctcaaatccag |
| | | actgaccatcatcaaggacaactccaagagccaagttttcttaaaaatgaac |
| | | agtctgcaaactgatgacacagccatttactactgtgccaaacattattact |
| | | acggtggtagctatgctatggactactggggtcaaggaacctcagtcaccgt |
| | | ctcctca |
| 413 | CARA- | DIQMTQTTSSLSASLGDRVTISCRASQDISKYLNWYQQKPDGTVKLLIYHTS |
| | CD 19 scFv | RLHSGVPSRFSGSGSGTDYSLTISNLEQEDIATYFCQQGNTLPYTFGGGTKL |
| | amino acid | EITGSTSGSGKPGSGEGSTKGEVKLQESGPGLVAPSQSLSVTCTVSGVSLPD |
| | sequence; | YGVSWIRQPPRKGLEWLGVIWGSETTYYNSALKSRLTIIKDNSKSQVFLKMN |
| | no leader | SLQTDDTAIYYCAKHYYYGGSYAMDYWGQGTSVTVSS |
| CAR B | | |
| 414 | CARB-full | ATGCTGCTGCTGGTGACCAGCCTGCTGCTGTGCGAGCTGCCCCACCCCGCCT |
| | with leader | TTCTGCTGATCCCCGACATCCAGATGACCCAGACCACCTCCAGCCTGAGCGC |
| | underlined | CAGCCTGGGCGACCGGGTGACCATCAGCTGCCGGGCCAGCCAGGACATCAGC |
| | nucleotide | AAGTACCTGAACTGGTATCAGCAGAAGCCCGACGGCACCGTCAAGCTGCTGA |
| | sequence; | TCTACCACACCAGCCGGCTGCACAGCGGCGTGCCCAGCCGGTTTAGCGGCAG |
| | | CGGCTCCGGCACCGACTACAGCCTGACCATCTCCAACCTGGAACAGGAAGAT |
| | | ATCGCCACCTACTTTTGCCAGCAGGGCAACACACTGCCCTACACCTTTGGCG |
| | | GCGGAACAAAGCTGGAAATCACCGGCAGCACCTCCGGCAGCGGCAAGCCTGG |
| | | CAGCGGCGAGGGCAGCACCAAGGGCGAGGTGAAGCTGCAGGAAAGCGGCCCT |
| | | GGCCTGGTGGCCCCCAGCCAGAGCCTGAGCGTGACCTGCACCGTGAGCGGCG |
| | | TGAGCCTGCCCGACTACGGCGTGAGCTGGATCCGGCAGCCCCCCAGGAAGGG |
| | | CCTGGAATGGCTGGGCGTGATCTGGGGCAGCGAGACCACCTACTACAACAGC |
| | | GCCCTGAAGAGCCGGCTGACCATCATCAAGGACAACAGCAAGAGCCAGGTGT |
| | | TCCTGAAGATGAACAGCCTGCAGACCGACGACACCGCCATCTACTACTGCGC |
| | | CAAGCACTACTACTACGGCGGCAGCTACGCCATGGACTACTGGGGCCAGGGC |
| | | ACCAGCGTGACCGTGAGCAGCGAATCTAAGTACGGACCGCCCTGCCCCCCTT |
| | | GCCCTATGTTCTGGGTGCTGGTGGTGGTCGGAGGCGTGCTGGCCTGCTACAG |
| | | CCTGCTGGTCACCGTGGCCTTCATCATCTTTTGGGTGAAACGGGGCAGAAAG |
| | | AAACTCCTGTATATATTCAAACAACCATTTATGAGACCAGTACAAACTACTC |
| | | AAGAGGAAGATGGCTGTAGCTGCCGATTTCCAGAAGAAGAAGAAGGAGGATG |
| | | TGAACTGCGGGTGAAGTTCAGCAGAAGCGCCGACGCCCCTGCCTACCAGCAG |
| | | GGCCAGAATCAGCTGTACAACGAGCTGAACCTGGGCAGAAGGGAAGAGTACG |
| | | ACGTCCTGGATAAGCGGAGAGGCCGGGACCCTGAGATGGGCGGCAAGCCTCG |
| | | GCGGAAGAACCCCCAGGAAGGCCTGTATAACGAACTGCAGAAAGACAAGATG |
| | | GCCGAGGCCTACAGCGAGATCGGCATGAAGGGCGAGCGGAGGCGGGGCAAGG |
| | | GCCACGACGGCCTGTATCAGGGCCTGTCCACCGCCACCAAGGATACCTACGA |
| | | CGCCCTGCACATGCAGGCCCTGCCCCCAAGG |
| 415 | CARB-full | MLLLVTSLLLCELPHPAFLLIPDIQMTQTTSSLSASLGDRVTISCRASQDIS |
| | transgene | KYLNWYQQKPDGTVKLLIYHTSRLHSGVPSRFSGSGSGTDYSLTISNLEQED |
| | amino acid | IATYFCQQGNTLPYTFGGGTKLEITGSTSGSGKPGSGEGSTKGEVKLQESGP |
| | sequence; | GLVAPSQSLSVTCTVSGVSLPDYGVSWIRQPPRKGLEWLGVIWGSETTYYNS |
| | with leader | ALKSRLTIIKDNSKSQVFLKMNSLQTDDTAIYYCAKHYYYGGSYAMDYWGQG |
| | underlined | TSVTVSSESKYGPPCPPCPMFWVLVWGGVLACYSLLVTVAFIIFWVKRGRK |
| | | KLLYIFKQPFMRPVQTTQEEDGCSCRFPEEEEGGCELRVKFSRSADAPAYQQ |
| | | GQNQLYNELNLGRREEYDVLDKRRGRDPEMGGKPRRKNPQEGLYNELQKDKM |
| | | AEAYSEIGMKGERRRGKGHDGLYQGLSTATKDTYDALHMQALPPR |
| 416 | CARB- | ATGCTGCTGCTGGTGACCAGCCTGCTGCTGTGCGAGCTGCCCCACCCCGCCT |
| | sequence; | TTCTGCTGATCCCCGACATCCAGATGACCCAGACCACCTCCAGCCTGAGCGC |
| | with leader | CAGCCTGGGCGACCGGGTGACCATCAGCTGCCGGGCCAGCCAGGACATCAGC |
| | underlined | AAGTACCTGAACTGGTATCAGCAGAAGCCCGACGGCACCGTCAAGCTGCTGA |
| | CD19 scFv | TCTACCACACCAGCCGGCTGCACAGCGGCGTGCCCAGCCGGTTTAGCGGCAG |
| | nucleotide | CGGCTCCGGCACCGACTACAGCCTGACCATCTCCAACCTGGAACAGGAAGAT |
| | | ATCGCCACCTACTTTTGCCAGCAGGGCAACACACTGCCCTACACCTTTGGCG |
| | | GCGGAACAAAGCTGGAAATCACCGGCAGCACCTCCGGCAGCGGCAAGCCTGG |
| | | CAGCGGCGAGGGCAGCACCAAGGGCGAGGTGAAGCTGCAGGAAAGCGGCCCT |
| | | GGCCTGGTGGCCCCCAGCCAGAGCCTGAGCGTGACCTGCACCGTGAGCGGCG |
| | | TGAGCCTGCCCGACTACGGCGTGAGCTGGATCCGGCAGCCCCCCAGGAAGGG |
| | | CCTGGAATGGCTGGGCGTGATCTGGGGCAGCGAGACCACCTACTACAACAGC |
| | | GCCCTGAAGAGCCGGCTGACCATCATCAAGGACAACAGCAAGAGCCAGGTGT |
| | | TCCTGAAGATGAACAGCCTGCAGACCGACGACACCGCCATCTACTACTGCGC |
| | | CAAGCACTACTACTACGGCGGCAGCTACGCCATGGACTACTGGGGCCAGGGC |
| | | ACCAGCGTGACCGTGAGCAGC |
| 417 | CARB- | MLLLVTSLLLCELPHPAFLLIPDIQMTQTTSSLSASLGDRVTISCRASQDIS |
| | CD19 scFv | KYLNWYQQKPDGTVKLLIYHTSRLHSGVPSRFSGSGSGTDYSLTISNLEQED |
| | amino acid | IATYFCQQGNTLPYTFGGGTKLEITGSTSGSGKPGSGEGSTKGEVKLQESGP |
| | sequence; | GLVAPSQSLSVTCTVSGVSLPDYGVSWIRQPPRKGLEWLGVIWGSETTYYNS |
| | with leader | ALKSRLTIIKDNSKSQVFLKMNSLQTDDTAIYYCAKHYYYGGSYAMDYWGQG |
| | underlined | TSVTVSS |
| 418 | CARB-full | GACATCCAGATGACCCAGACCACCTCCAGCCTGAGCGCCAGCCTGGGCGACC |
| | nucleotide | GGGTGACCATCAGCTGCCGGGCCAGCCAGGACATCAGCAAGTACCTGAACTG |
| | sequence; | GTATCAGCAGAAGCCCGACGGCACCGTCAAGCTGCTGATCTACCACACCAGC |
| | no leader | CGGCTGCACAGCGGCGTGCCCAGCCGGTTTAGCGGCAGCGGCTCCGGCACCG |
| | | ACTACAGCCTGACCATCTCCAACCTGGAACAGGAAGATATCGCCACCTACTT |
| | | TTGCCAGCAGGGCAACACACTGCCCTACACCTTTGGCGGCGGAACAAAGCTG |
| | | GAAATCACCGGCAGCACCTCCGGCAGCGGCAAGCCTGGCAGCGGCGAGGGCA |
| | | GCACCAAGGGCGAGGTGAAGCTGCAGGAAAGCGGCCCTGGCCTGGTGGCCCC |
| | | CAGCCAGAGCCTGAGCGTGACCTGCACCGTGAGCGGCGTGAGCCTGCCCGAC |
| | | TACGGCGTGAGCTGGATCCGGCAGCCCCCCAGGAAGGGCCTGGAATGGCTGG |
| | | GCGTGATCTGGGGCAGCGAGACCACCTACTACAACAGCGCCCTGAAGAGCCG |
| | | GCTGACCATCATCAAGGACAACAGCAAGAGCCAGGTGTTCCTGAAGATGAAC |
| | | AGCCTGCAGACCGACGACACCGCCATCTACTACTGCGCCAAGCACTACTACT |
| | | ACGGCGGCAGCTACGCCATGGACTACTGGGGCCAGGGCACCAGCGTGACCGT |
| | | GAGCAGCGAATCTAAGTACGGACCGCCCTGCCCCCCTTGCCCTATGTTCTGG |
| | | GTGCTGGTGGTGGTCGGAGGCGTGCTGGCCTGCTACAGCCTGCTGGTCACCG |
| | | TGGCCTTCATCATCTTTTGGGTGAAACGGGGCAGAAAGAAACTCCTGTATAT |
| | | ATTCAAACAACCATTTATGAGACCAGTACAAACTACTCAAGAGGAAGATGGC |
| | | TGTAGCTGCCGATTTCCAGAAGAAGAAGAAGGAGGATGTGAACTGCGGGTGA |
| | | AGTTCAGCAGAAGCGCCGACGCCCCTGCCTACCAGCAGGGCCAGAATCAGCT |
| | | GTACAACGAGCTGAACCTGGGCAGAAGGGAAGAGTACGACGTCCTGGATAAG |
| | | CGGAGAGGCCGGGACCCTGAGATGGGCGGCAAGCCTCGGCGGAAGAACCCCC |
| | | AGGAAGGCCTGTATAACGAACTGCAGAAAGACAAGATGGCCGAGGCCTACAG |
| | | CGAGATCGGCATGAAGGGCGAGCGGAGGCGGGGCAAGGGCCACGACGGCCTG |
| | | TATCAGGGCCTGTCCACCGCCACCAAGGATACCTACGACGCCCTGCACATGC |
| | | AGGCCCTGCCCCCAAGG |
| 419 | CARB-full | DIQMTQTTSSLSASLGDRVTISCRASQDISKYLNWYQQKPDGTVKLLIYHTS |
| | amino acid | RLHSGVPSRFSGSGSGTDYSLTISNLEQEDIATYFCQQGNTLPYTFGGGTKL |
| | transgene | EITGSTSGSGKPGSGEGSTKGEVKLQESGPGLVAPSQSLSVTCTVSGVSLPD |
| | sequence; | YGVSWIRQPPRKGLEWLGVIWGSETTYYNSALKSRLTIIKDNSKSQVFLKMN |
| | no leader | SLQTDDTAIYYCAKHYYYGGSYAMDYWGQGTSVTVSSESKYGPPCPPCPMFW |
| | | VLVWGGVLACYSLLVTVAFIIFWVKRGRKKLLYIFKQPFMRPVQTTQEEDG |
| | | CSCRFPEEEEGGCELRVKFSRSADAPAYQQGQNQLYNELNLGRREEYDVLDK |
| | | RRGRDPEMGGKPRRKNPQEGLYNELQKDKMAEAYSEIGMKGERRRGKGHDGL |
| | | YQGLSTATKDTYDALHMQALPPR |
| 420 | CARB- | GACATCCAGATGACCCAGACCACCTCCAGCCTGAGCGCCAGCCTGGGCGACC |
| | CD19 scFv | GGGTGACCATCAGCTGCCGGGCCAGCCAGGACATCAGCAAGTACCTGAACTG |
| | sequence; | GTATCAGCAGAAGCCCGACGGCACCGTCAAGCTGCTGATCTACCACACCAGC |
| | no leader | CGGCTGCACAGCGGCGTGCCCAGCCGGTTTAGCGGCAGCGGCTCCGGCACCG |
| | | ACTACAGCCTGACCATCTCCAACCTGGAACAGGAAGATATCGCCACCTACTT |
| | | TTGCCAGCAGGGCAACACACTGCCCTACACCTTTGGCGGCGGAACAAAGCTG |
| | | GAAATCACCGGCAGCACCTCCGGCAGCGGCAAGCCTGGCAGCGGCGAGGGCA |
| | | GCACCAAGGGCGAGGTGAAGCTGCAGGAAAGCGGCCCTGGCCTGGTGGCCCC |
| | | CAGCCAGAGCCTGAGCGTGACCTGCACCGTGAGCGGCGTGAGCCTGCCCGAC |
| | | TACGGCGTGAGCTGGATCCGGCAGCCCCCCAGGAAGGGCCTGGAATGGCTGG |
| | | GCGTGATCTGGGGCAGCGAGACCACCTACTACAACAGCGCCCTGAAGAGCCG |
| | | GOTGACCATCATCAAGGACAACAGCAAGAGCCAGGTGTTCCTGAAGATGAAC |
| | | AGCCTGCAGACCGACGACACCGCCATCTACTACTGCGCCAAGCACTACTACT |
| | | ACGGCGGCAGCTACGCCATGGACTACTGGGGCCAGGGCACCAGCGTGACCGT |
| | | GAGCAGC |
| 413 | CARB- | DIQMTQTTSSLSASLGDRVTISCRASQDISKYLNWYQQKPDGTVKLLIYHTS |
| | CD19 scFv | RLHSGVPSRFSGSGSGTDYSLTISNLEQEDIATYFCQQGNTLPYTFGGGTKL |
| | sequence; | EITGSTSGSGKPGSGEGSTKGEVKLQESGPGLVAPSQSLSVTCTVSGVSLPD |
| | no leader | YGVSWIRQPPRKGLEWLGVIWGSETTYYNSALKSRLTIIKDNSKSQVFLKMN |
| | | SLQTDDTAIYYCAKHYYYGGSYAMDYWGQGTSVTVSS |

### BCMA CAR

In some embodiments, the CAR-expressing cell described herein is a BCMA CAR-expressing cell (for example, a cell expressing a CAR that binds to human BCMA). Exemplary BCMA CARs can include sequences disclosed in Table 1 or 16 of WO2016/014565, incorporated herein by reference. The BCMA CAR construct can include an optional leader sequence; an optional hinge domain, for example, a CD8 hinge domain; a transmembrane domain, for example, a CD8 transmembrane domain; an intracellular domain, for example, a 4-1BB intracellular domain; and a functional signaling domain, for example, a CD3 zeta domain. In certain embodiments, the domains are contiguous and in the same reading frame to form a single fusion protein. In other embodiments, the domains are in separate polypeptides, for example, as in an RCAR molecule as described herein.

In some embodiments, the BCMA CAR molecule includes one or more CDRs, VH, VL, scFv, or full-length sequences of BCMA-1, BCMA-2, BCMA-3, BCMA-4, BCMA-5, BCMA-6, BCMA-7, BCMA-8, BCMA-9, BCMA-10, BCMA-11, BCMA-12, BCMA-13, BCMA-14, BCMA-15, 149362, 149363, 149364, 149365, 149366, 149367, 149368, 149369, BCMA_EBB-C1978-A4, BCMA_EBB-C1978-G1, BCMA_EBB-C1979-C1, BCMA_EBB-C1978-C7, BCMA_EBB-C1978-D10, BCMA_EBB-C1979-C12, BCMA_EBB-C1980-G4, BCMA_EBB-C1980-D2, BCMA_EBB-C1978-A10, BCMA_EBB-C1978-D4, BCMA_EBB-C1980-A2, BCMA_EBB-C1981-C3, BCMA_EBB-C1978-G4, A7D12.2, C11D5.3, C12A3.2, or C13F12.1 disclosed in WO2016/014565, or a sequence substantially (for example, 95-99%) identical thereto.

Additional exemplary BCMA-targeting sequences that can be used in the anti-BCMA CAR constructs are disclosed in WO 2017/021450, WO 2017/011804, WO 2017/025038, WO 2016/090327, WO 2016/130598, WO 2016/210293, WO 2016/090320, WO 2016/014789, WO 2016/094304, WO 2016/154055, WO 2015/166073, WO 2015/188119, WO 2015/158671, U.S. Pat. Nos. 9,243,058, 8,920,776, 9,273,141, 7,083,785, 9,034,324, US 2007/0049735, US 2015/0284467, US 2015/0051266, US 2015/0344844, US 2016/0131655, US 2016/0297884, US 2016/0297885, US 2017/0051308, US 2017/0051252, US 2017/0051252, WO 2016/020332, WO 2016/087531, WO 2016/079177, WO 2015/172800, WO 2017/008169, U.S. Pat. No. 9,340,621, US 2013/0273055, US 2016/0176973, US 2015/0368351, US 2017/0051068, US 2016/0368988, and US 2015/0232557, herein incorporated by reference in their entirety. In some embodiments, additional exemplary BCMA CAR constructs are generated using the VH and VL sequences from PCT Publication WO2012/0163805 (the contents of which are hereby incorporated by reference in its entirety).

In some embodiments, BCMA CARs comprise a sequence, for example, a CDR, VH, VL, scFv, or full-CAR sequence, disclosed in Tables 3-15, or a sequence having at least 80%, 85%, 90%, 95%, or 99% identity thereto. In some embodiments, the antigen binding domain comprises a human antibody or a human antibody fragment. In some embodiments, the human anti-BCMA binding domain comprises one or more (for example, all three) LC CDR1, LC CDR2, and LC CDR3 of a human anti-BCMA binding domain described herein (for example, in Tables 3-10 and 12-15), and/or one or more (for example, all three) HC CDR1, HC CDR2, and HC CDR3 of a human anti-BCMA binding domain described herein (for example, in Tables 3-10 and 12-15). In some embodiments, the human anti-BCMA binding domain comprises a human VL described herein (for example, in Tables 3, 7, and 12) and/or a human VH described herein (for example, in Tables 3, 7, and 12). In some embodiments, the anti-BCMA binding domain is a scFv comprising a VL and a VH of an amino acid sequence of Tables 3, 7, and 12. In some embodiments, the anti-BCMA binding domain (for example, an scFv) comprises: a VL comprising an amino acid sequence having at least one, two or three modifications (for example, substitutions, for example, conservative substitutions) but not more than 30, 20 or 10 modifications (for example, substitutions, for example, conservative substitutions) of an amino acid sequence provided in Tables 3, 7, and 12, or a sequence with 95-99% identity with an amino acid sequence of Tables 3, 7, and 12, and/or a VH comprising an amino acid sequence having at least one, two or three modifications (for example, substitutions, for example, conservative substitutions) but not more than 30, 20 or 10 modifications (for example, substitutions, for example, conservative substitutions) of an amino acid sequence provided in Tables 3, 7, and 12, or a sequence with 95-99% identity to an amino acid sequence 5 of Tables 3, 7, and 12.

TABLE 3
Amino acid and nucleic acid sequences of exemplary
PALLAS-derived anti-BCMA mlecules

| | Name/ | |
|---|---|---|
| SEQ ID NO | Description | Sequence |
| R1B6 | | |
| SEQ ID | HCDR1 | SYAMS |
| NO: 44 | (Kabat) | |
| SEQ ID | HCDR2 | AISGSGGSTYYADSVKG |
| NO: 45 | (Kabat) | |
| SEQ ID | HCDR3 | REWVPYDVSWYFDY |
| NO: 46 | (Kabat) | |
| SEQ ID | HCDR1 | GFTFSSY |
| NO: 47 | (Chothia) | |
| SEQ ID | HCDR2 | SGSGGS |
| NO: 48 | (Chothia) | |
| SEQ ID | HCDR3 | REWVPYDVSWYFDY |
| NO: 46 | (Chothia) | |
| SEQ ID | HCDR1 | GFTFSSYA |
| NO: 49 | (IMGT) | |
| SEQ ID | HCDR2 | ISGSGGST |
| NO: 50 | (IMGT) | |
| SEQ ID | HCDR3 | ARREWVPYDVSWYFDY |
| NO: 51 | (IMGT) | |
| SEQ ID | VH | EVQLLESGGGLVQPGGSLRLSCAASGFTFSSYAMSWVRQAPGKGL |
| NO: 52 | | EWVSAISGSGGSTYYADSVKGRFTISRDNSKNTLYLQMNSLRAED |
| | | TAVYYCARREWVPYDVSWYFDYWGQGTLVTVSS |
| SEQ ID | DNA VH | GAAGTGCAGTTGCTGGAGTCAGGCGGAGGACTGGTGCAGCCCG |
| NO: 53 | | GAGGATCGCTTCGCTTGAGCTGCGCAGCCTCAGGCTTTACCTTC |
| | | TCCTCCTACGCCATGTCCTGGGTCAGACAGGCTCCCGGGAAGG |
| | | GACTGGAATGGGTGTCCGCCATTAGCGGTTCCGGCGGAAGCAC |
| | | TTACTATGCCGACTCTGTGAAGGGCCGCTTCACTATCTCCCGGG |
| | | ACAACTCCAAGAACACCCTGTATCTCCAAATGAATTCCCTGAGG |
| | | GCCGAAGATACCGCGGTGTACTACTGCGCTAGACGGGAGTGGG |
| | | TGCCCTACGATGTCAGCTGGTACTTCGACTACTGGGGACAGGGC |
| | | ACTCTCGTGACTGTGTCCTCC |
| SEQ ID | LCDR1 | RASQSISSYLN |
| NO: 54 | (Kabat) | |
| SEQ ID | LCDR2 | AASSLQS |
| NO: 55 | (Kabat) | |
| SEQ ID | LCDR3 | QQSYSTPLT |
| NO: 56 | (Kabat) | |
| SEQ ID | LCDR1 | SQSISSY |
| NO: 57 | (Chothia) | |
| SEQ ID | LCDR2 | AAS |
| NO: 58 | (Chothia) | |
| SEQ ID | LCDR3 | SYSTPL |
| NO: 59 | (Chothia) | |
| SEQ ID | LCDR1 | QSISSY |
| NO: 60 | (IMGT) | |
| SEQ ID | LCDR2 | AAS |
| NO: 58 | (IMGT) | |
| SEQ ID | LCDR3 | QQSYSTPLT |
| NO: 56 | (IMGT) | |
| SEQ ID | VL | DIQMTQSPSSLSASVGDRVTITCRASQSISSYLNWYQQKPGKAPKL |
| NO: 61 | | LIYAASSLQSGVPSRFSGSGSGTDFTLTISSLQPEDFATYYCQQSYS |
| | | TPLTFGQGTKVEIK |
| SEQ ID | DNA VL | GACATTCAAATGACTCAGTCCCCGTCCTCCCTCTCCGCCTCCGT |
| NO: 62 | | GGGAGATCGCGTCACGATCACGTGCAGGGCCAGCCAGAGCATC |
| | | TCCAGCTACCTGAACTGGTACCAGCAGAAGCCAGGGAAGGCAC |
| | | CGAAGCTCCTGATCTACGCCGCTAGCTCGCTGCAGTCCGGCGTC |
| | | CCTTCACGGTTCTCGGGATCGGGCTCAGGCACCGACTTCACCCT |
| | | GACCATTAGCAGCCTGCAGCCGGAGGACTTCGCGACATACTAC |
| | | TGTCAGCAGTCATACTCCACCCCTCTGACCTTCGGCCAAGGGAC |
| | | CAAAGTGGAGATCAAG |
| SEQ ID | Linker | GGGGSGGGGSGGGGSGGGGS |
| NO: 63 | | |
| SEQ ID | scFv (VH- | EVQLLESGGGLVQPGGSLRLSCAASGFTFSSYAMSWVRQAPGKGL |
| NO: 64 | linker-VL) | EWVSAISGSGGSTYYADSVKGRFTISRDNSKNTLYLQMNSLRAED |
| | | TAVYYCARREWVPYDVSWYFDYWGQGTLVTVSSGGGGSGGGGS |
| | | GGGGSGGGGSDIQMTQSPSSLSASVGDRVTITCRASQSISSYLNWY |
| | | QQKPGKAPKLLIYAASSLQSGVPSRFSGSGSGTDFTLTISSLQPEDF |
| | | ATYYCQQSYSTPLTFGQGTKVEIK |
| SEQ ID | DNA scFv | GAAGTGCAGTTGCTGGAGTCAGGCGGAGGACTGGTGCAGCCCG |
| NO: 65 | | GAGGATCGCTTCGCTTGAGCTGCGCAGCCTCAGGCTTTACCTTC |
| | | TCCTCCTACGCCATGTCCTGGGTCAGACAGGCTCCCGGGAAGG |
| | | GACTGGAATGGGTGTCCGCCATTAGCGGTTCCGGCGGAAGCAC |
| | | TTACTATGCCGACTCTGTGAAGGGCCGCTTCACTATCTCCCGGG |
| | | ACAACTCCAAGAACACCCTGTATCTCCAAATGAATTCCCTGAGG |
| | | GCCGAAGATACCGCGGTGTACTACTGCGCTAGACGGGAGTGGG |
| | | TGCCCTACGATGTCAGCTGGTACTTCGACTACTGGGGACAGGGC |
| | | ACTCTCGTGACTGTGTCCTCCGGTGGTGGTGGATCGGGGGGTGG |
| | | TGGTTCGGGCGGAGGAGGATCTGGAGGAGGAGGGTCGGACATT |
| | | CAAATGACTCAGTCCCCGTCCTCCCTCTCCGCCTCCGTGGGAGA |
| | | TCGCGTCACGATCACGTGCAGGGCCAGCCAGAGCATCTCCAGC |
| | | TACCTGAACTGGTACCAGCAGAAGCCAGGGAAGGCACCGAAGC |
| | | TCCTGATCTACGCCGCTAGCTCGCTGCAGTCCGGCGTCCCTTCA |
| | | CGGTTCTCGGGATCGGGCTCAGGCACCGACTTCACCCTGACCAT |
| | | TAGCAGCCTGCAGCCGGAGGACTTCGCGACATACTACTGTCAG |
| | | CAGTCATACTCCACCCCTCTGACCTTCGGCCAAGGGACCAAAGT |
| | | GGAGATCAAG |
| SEQ ID | Full CAR | EVQLLESGGGLVQPGGSLRLSCAASGFTFSSYAMSWVRQAPGKGL |
| NO: 66 | amino acid | EWVSAISGSGGSTYYADSVKGRFTISRDNSKNTLYLQMNSLRAED |
| | sequence | TAVYYCARREWVPYDVSWYFDYWGQGTLVTVSSGGGGSGGGGS |
| | | GGGGSGGGGSDIQMTQSPSSLSASVGDRVTITCRASQSISSYLNWY |
| | | QQKPGKAPKLLIYAASSLQSGVPSRFSGSGSGTDFTLTISSLQPEDF |
| | | ATYYCQQSYSTPLTFGQGTKVEIKTTTPAPRPPTPAPTIASQPLSLRP |
| | | EACRPAAGGAVHTRGLDFACDIYIWAPLAGTCGVLLLSLVITLYC |
| | | KRGRKKLLYIFKQPFMRPVQTTQEEDGCSCRFPEEEEGGCELRVKF |
| | | SRSADAPAYQQGQNQLYNELNLGRREEYDVLDKRRGRDPEMGG |
| | | KPRRKNPQEGLYNELQKDKMAEAYSEIGMKGERRRGKGHDGLY |
| | | QGLSTATKDTYDALHMQALPPR |
| SEQ ID | Full CAR | GAAGTGCAGTTGCTGGAGTCAGGCGGAGGACTGGTGCAGCCCG |
| NO: 67 | DNA | GAGGATCGCTTCGCTTGAGCTGCGCAGCCTCAGGCTTTACCTTC |
| | sequence | TCCTCCTACGCCATGTCCTGGGTCAGACAGGCTCCCGGGAAGG |
| | | GACTGGAATGGGTGTCCGCCATTAGCGGTTCCGGCGGAAGCAC |
| | | TTACTATGCCGACTCTGTGAAGGGCCGCTTCACTATCTCCCGGG |
| | | ACAACTCCAAGAACACCCTGTATCTCCAAATGAATTCCCTGAGG |
| | | GCCGAAGATACCGCGGTGTACTACTGCGCTAGACGGGAGTGGG |
| | | TGCCCTACGATGTCAGCTGGTACTTCGACTACTGGGGACAGGGC |
| | | ACTCTCGTGACTGTGTCCTCCGGTGGTGGTGGATCGGGGGGTGG |
| | | TGGTTCGGGCGGAGGAGGATCTGGAGGAGGAGGGTCGGACATT |
| | | CAAATGACTCAGTCCCCGTCCTCCCTCTCCGCCTCCGTGGGAGA |
| | | TCGCGTCACGATCACGTGCAGGGCCAGCCAGAGCATCTCCAGC |
| | | TACCTGAACTGGTACCAGCAGAAGCCAGGGAAGGCACCGAAGC |
| | | TCCTGATCTACGCCGCTAGCTCGCTGCAGTCCGGCGTCCCTTCA |
| | | CGGTTCTCGGGATCGGGCTCAGGCACCGACTTCACCCTGACCAT |
| | | TAGCAGCCTGCAGCCGGAGGACTTCGCGACATACTACTGTCAG |
| | | CAGTCATACTCCACCCCTCTGACCTTCGGCCAAGGGACCAAAGT |
| | | GGAGATCAAGACCACTACCCCAGCACCGAGGCCACCCACCCCG |
| | | GCTCCTACCATCGCCTCCCAGCCTCTGTCCCTGCGTCCGGAGGC |
| | | ATGTAGACCCGCAGCTGGTGGGGCCGTGCATACCCGGGGTCTT |
| | | GACTTCGCCTGCGATATCTACATTTGGGCCCCTCTGGCTGGTAC |
| | | TTGCGGGGTCCTGCTGCTTTCACTCGTGATCACTCTTTACTGTAA |
| | | GCGCGGTCGGAAGAAGCTGCTGTACATCTTTAAGCAACCCTTCA |
| | | TGAGGCCTGTGCAGACTACTCAAGAGGAGGACGGCTGTTCATG |
| | | CCGGTTCCCAGAGGAGGAGGAAGGCGGCTGCGAACTGCGCGTG |
| | | AAATTCAGCCGCAGCGCAGATGCTCCAGCCTACCAGCAGGGGC |
| | | AGAACCAGCTCTACAACGAACTCAATCTTGGTCGGAGAGAGGA |
| | | GTACGACGTGCTGGACAAGCGGAGAGGACGGGACCCAGAAAT |
| | | GGGCGGGAAGCCGCGCAGAAAGAATCCCCAAGAGGGCCTGTA |
| | | CAACGAGCTCCAAAAGGATAAGATGGCAGAAGCCTATAGCGAG |
| | | ATTGGTATGAAAGGGGAACGCAGAAGAGGCAAAGGCCACGAC |
| | | GGACTGTACCAGGGACTCAGCACCGCCACCAAGGACACCTATG |
| | | ACGCTCTTCACATGCAGGCCCTGCCGCCTCGG |
| R1F2 | | |
| SEQ ID | HCDR1 | SYAMS |
| NO: 44 | (Kabat) | |
| SEQ ID | HCDR2 | AISGSGGSTYYADSVKG |
| NO: 45 | (Kabat) | |
| SEQ ID | HCDR3 | REWWYDDWYLDY |
| NO: 68 | (Kabat) | |
| SEQ ID | HCDR1 | GFTFSSY |
| NO: 47 | (Chothia) | |
| SEQ ID | HCDR2 | SGSGGS |
| NO: 48 | (Chothia) | |
| SEQ ID | HCDR3 | REWWYDDWYLDY |
| NO: 68 | (Chothia) | |
| SEQ ID | HCDR1 | GFTFSSYA |
| NO: 49 | (IMGT) | |
| SEQ ID | HCDR2 | ISGSGGST |
| NO: 50 | (IMGT) | |
| SEQ ID | HCDR3 | ARREWWYDDWYLDY |
| NO: 69 | (IMGT) | |
| SEQ ID | VH | EVQLLESGGGLVQPGGSLRLSCAASGFTFSSYAMSWVRQAPGKGL |
| NO: 70 | | EWVSAISGSGGSTYYADSVKGRFTISRDNSKNTLYLQMNSLRAED |
| | | TAVYYCARREWWYDDWYLDYWGQGTLVTVSS |
| SEQ ID | DNA VH | GAAGTGCAGTTGCTGGAGTCAGGCGGAGGACTGGTGCAGCCCG |
| NO: 71 | | GAGGATCGCTTCGCTTGAGCTGCGCAGCCTCAGGCTTTACCTTC |
| | | TCCTCCTACGCCATGTCCTGGGTCAGACAGGCTCCCGGGAAGG |
| | | GACTGGAATGGGTGTCCGCCATTAGCGGTTCCGGCGGAAGCAC |
| | | TTACTATGCCGACTCTGTGAAGGGCCGCTTCACTATCTCCCGGG |
| | | ACAACTCCAAGAACACCCTGTATCTCCAAATGAATTCCCTGAGG |
| | | GCCGAAGATACCGCGGTGTACTACTGCGCTAGACGGGAGTGGT |
| | | GGTACGACGATTGGTACCTGGACTACTGGGGACAGGGCACTCT |
| | | CGTGACTGTGTCCTCC |
| SEQ ID | LCDR1 | RASQSISSYLN |
| NO: 54 | (Kabat) | |
| SEQ ID | LCDR2 | AASSLQS |
| NO: 55 | (Kabat) | |
| SEQ ID | LCDR3 | QQSYSTPLT |
| NO: 56 | (Kabat) | |
| SEQ ID | LCDR1 | SQSISSY |
| NO: 57 | (Chothia) | |
| SEQ ID | LCDR2 | AAS |
| NO: 58 | (Chothia) | |
| SEQ ID | LCDR3 | SYSTPL |
| NO: 59 | (Chothia) | |
| SEQ ID | LCDR1 | QSISSY |
| NO: 60 | (IMGT) | |
| SEQ ID | LCDR2 | AAS |
| NO: 58 | (IMGT) | |
| SEQ ID | LCDR3 | QQSYSTPLT |
| NO: 56 | (IMGT) | |
| SEQ ID | VL | DIQMTQSPSSLSASVGDRVTITCRASQSISSYLNWYQQKPGKAPKL |
| NO: 61 | | LIYAASSLQSGVPSRFSGSGSGTDFTLTISSLQPEDFATYYCQQSYS |
| | | TPLTFGQGTKVEIK |
| SEQ ID | DNA VL | GACATTCAAATGACTCAGTCCCCGTCCTCCCTCTCCGCCTCCGT |
| NO: 62 | | GGGAGATCGCGTCACGATCACGTGCAGGGCCAGCCAGAGCATC |
| | | TCCAGCTACCTGAACTGGTACCAGCAGAAGCCAGGGAAGGCAC |
| | | CGAAGCTCCTGATCTACGCCGCTAGCTCGCTGCAGTCCGGCGTC |
| | | CCTTCACGGTTCTCGGGATCGGGCTCAGGCACCGACTTCACCCT |
| | | GACCATTAGCAGCCTGCAGCCGGAGGACTTCGCGACATACTAC |
| | | TGTCAGCAGTCATACTCCACCCCTCTGACCTTCGGCCAAGGGAC |
| | | CAAAGTGGAGATCAAG |
| SEQ ID | Linker | GGGGSGGGGSGGGGSGGGGS |
| NO: 63 | | |
| SEQ ID | scFv (VH- | EVQLLESGGGLVQPGGSLRLSCAASGFTFSSYAMSWVRQAPGKGL |
| NO: 72 | linker-VL) | EWVSAISGSGGSTYYADSVKGRFTISRDNSKNTLYLQMNSLRAED |
| | | TAVYYCARREWWYDDWYLDYWGQGTLVTVSSGGGGSGGGGSG |
| | | GGGSGGGGSDIQMTQSPSSLSASVGDRVTITCRASQSISSYLNWYQ |
| | | QKPGKAPKLLIYAASSLQSGVPSRFSGSGSGTDFTLTISSLQPEDFA |
| | | TYYCQQSYSTPLTFGQGTKVEIK |
| SEQ ID | DNA scFv | GAAGTGCAGTTGCTGGAGTCAGGCGGAGGACTGGTGCAGCCCG |
| NO: 73 | | GAGGATCGCTTCGCTTGAGCTGCGCAGCCTCAGGCTTTACCTTC |
| | | TCCTCCTACGCCATGTCCTGGGTCAGACAGGCTCCCGGGAAGG |
| | | GACTGGAATGGGTGTCCGCCATTAGCGGTTCCGGCGGAAGCAC |
| | | TTACTATGCCGACTCTGTGAAGGGCCGCTTCACTATCTCCCGGG |
| | | ACAACTCCAAGAACACCCTGTATCTCCAAATGAATTCCCTGAGG |
| | | GCCGAAGATACCGCGGTGTACTACTGCGCTAGACGGGAGTGGT |
| | | GGTACGACGATTGGTACCTGGACTACTGGGGACAGGGCACTCT |
| | | CGTGACTGTGTCCTCCGGTGGTGGTGGATCGGGGGGTGGTGGTT |
| | | CGGGCGGAGGAGGATCTGGAGGAGGAGGGTCGGACATTCAAA |
| | | TGACTCAGTCCCCGTCCTCCCTCTCCGCCTCCGTGGGAGATCGC |
| | | GTCACGATCACGTGCAGGGCCAGCCAGAGCATCTCCAGCTACC |
| | | TGAACTGGTACCAGCAGAAGCCAGGGAAGGCACCGAAGCTCCT |
| | | GATCTACGCCGCTAGCTCGCTGCAGTCCGGCGTCCCTTCACGGT |
| | | TCTCGGGATCGGGCTCAGGCACCGACTTCACCCTGACCATTAGC |
| | | AGCCTGCAGCCGGAGGACTTCGCGACATACTACTGTCAGCAGT |
| | | CATACTCCACCCCTCTGACCTTCGGCCAAGGGACCAAAGTGGA |
| | | GATCAAG |
| SEQ ID | Full CAR | EVQLLESGGGLVQPGGSLRLSCAASGFTFSSYAMSWVRQAPGKGL |
| NO: 74 | amino acid | EWVSAISGSGGSTYYADSVKGRFTISRDNSKNTLYLQMNSLRAED |
| | sequence | TAVYYCARREWWYDDWYLDYWGQGTLVTVSSGGGGSGGGGSG |
| | | GGGSGGGGSDIQMTQSPSSLSASVGDRVTITCRASQSISSYLNWYQ |
| | | QKPGKAPKLLIYAASSLQSGVPSRFSGSGSGTDFTLTISSLQPEDFA |
| | | TYYCQQSYSTPLTFGQGTKVEIKTTTPAPRPPTPAPTIASQPLSLRPE |
| | | ACRPAAGGAVHTRGLDFACDIYIWAPLAGTCGVLLLSLVITLYCK |
| | | RGRKKLLYIFKQPFMRPVQTTQEEDGCSCRFPEEEEGGCELRVKFS |
| | | RSADAPAYQQGQNQLYNELNLGRREEYDVLDKRRGRDPEMGGK |
| | | PRRKNPQEGLYNELQKDKMAEAYSEIGMKGERRRGKGHDGLYQ |
| | | GLSTATKDTYDALHMQALPPR |
| SEQ ID | Full CAR | GAAGTGCAGTTGCTGGAGTCAGGCGGAGGACTGGTGCAGCCCG |
| NO: 75 | DNA | GAGGATCGCTTCGCTTGAGCTGCGCAGCCTCAGGCTTTACCTTC |
| | sequence | TCCTCCTACGCCATGTCCTGGGTCAGACAGGCTCCCGGGAAGG |
| | | GACTGGAATGGGTGTCCGCCATTAGCGGTTCCGGCGGAAGCAC |
| | | TTACTATGCCGACTCTGTGAAGGGCCGCTTCACTATCTCCCGGG |
| | | ACAACTCCAAGAACACCCTGTATCTCCAAATGAATTCCCTGAGG |
| | | GCCGAAGATACCGCGGTGTACTACTGCGCTAGACGGGAGTGGT |
| | | GGTACGACGATTGGTACCTGGACTACTGGGGACAGGGCACTCT |
| | | CGTGACTGTGTCCTCCGGTGGTGGTGGATCGGGGGGTGGTGGTT |
| | | CGGGCGGAGGAGGATCTGGAGGAGGAGGGTCGGACATTCAAA |
| | | TGACTCAGTCCCCGTCCTCCCTCTCCGCCTCCGTGGGAGATCGC |
| | | GTCACGATCACGTGCAGGGCCAGCCAGAGCATCTCCAGCTACC |
| | | TGAACTGGTACCAGCAGAAGCCAGGGAAGGCACCGAAGCTCCT |
| | | GATCTACGCCGCTAGCTCGCTGCAGTCCGGCGTCCCTTCACGGT |
| | | TCTCGGGATCGGGCTCAGGCACCGACTTCACCCTGACCATTAGC |
| | | AGCCTGCAGCCGGAGGACTTCGCGACATACTACTGTCAGCAGT |
| | | CATACTCCACCCCTCTGACCTTCGGCCAAGGGACCAAAGTGGA |
| | | GATCAAGACCACTACCCCAGCACCGAGGCCACCCACCCCGGCT |
| | | CCTACCATCGCCTCCCAGCCTCTGTCCCTGCGTCCGGAGGCATG |
| | | TAGACCCGCAGCTGGTGGGGCCGTGCATACCCGGGGTCTTGAC |
| | | TTCGCCTGCGATATCTACATTTGGGCCCCTCTGGCTGGTACTTG |
| | | CGGGGTCCTGCTGCTTTCACTCGTGATCACTCTTTACTGTAAGC |
| | | GCGGTCGGAAGAAGCTGCTGTACATCTTTAAGCAACCCTTCATG |
| | | AGGCCTGTGCAGACTACTCAAGAGGAGGACGGCTGTTCATGCC |
| | | GGTTCCCAGAGGAGGAGGAAGGCGGCTGCGAACTGCGCGTGAA |
| | | ATTCAGCCGCAGCGCAGATGCTCCAGCCTACCAGCAGGGGCAG |
| | | AACCAGCTCTACAACGAACTCAATCTTGGTCGGAGAGAGGAGT |
| | | ACGACGTGCTGGACAAGCGGAGAGGACGGGACCCAGAAATGG |
| | | GCGGGAAGCCGCGCAGAAAGAATCCCCAAGAGGGCCTGTACA |
| | | ACGAGCTCCAAAAGGATAAGATGGCAGAAGCCTATAGCGAGAT |
| | | TGGTATGAAAGGGGAACGCAGAAGAGGCAAAGGCCACGACGG |
| | | ACTGTACCAGGGACTCAGCACCGCCACCAAGGACACCTATGAC |
| | | GCTCTTCACATGCAGGCCCTGCCGCCTCGG |
| R1G5 | | |
| SEQ ID | HCDR1 | SYAMS |
| NO: 44 | (Kabat) | |
| SEQ ID | HCDR2 | AISGSGGSTYYADSVKG |
| NO: 45 | (Kabat) | |
| SEQ ID | HCDR3 | REWWGESWLFDY |
| NO: 76 | (Kabat) | |
| SEQ ID | HCDR1 | GFTFSSY |
| NO: 47 | (Chothia) | |
| SEQ ID | HCDR2 | SGSGGS |
| NO: 48 | (Chothia) | |
| SEQ ID | HCDR3 | REWWGESWLFDY |
| NO: 76 | (Chothia) | |
| SEQ ID | HCDR1 | GFTFSSYA |
| NO: 49 | (IMGT) | |
| SEQ ID | HCDR2 | ISGSGGST |
| NO: 50 | (IMGT) | |
| SEQ ID | HCDR3 | ARREWWGESWLFDY |
| NO: 77 | (IMGT) | |
| SEQ ID | VH | EVQLLESGGGLVQPGGSLRLSCAASGFTFSSYAMSWVRQAPGKGL |
| NO: 78 | | EWVSAISGSGGSTYYADSVKGRFTISRDNSKNTLYLQMNSLRAED |
| | | TAVYYCARREWWGESWLFDYWGQGTLVTVSS |
| SEQ ID | DNA VH | GAAGTGCAGTTGCTGGAGTCAGGCGGAGGACTGGTGCAGCCCG |
| NO: 79 | | GAGGATCGCTTCGCTTGAGCTGCGCAGCCTCAGGCTTTACCTTC |
| | | TCCTCCTACGCCATGTCCTGGGTCAGACAGGCTCCCGGGAAGG |
| | | GACTGGAATGGGTGTCCGCCATTAGCGGTTCCGGCGGAAGCAC |
| | | TTACTATGCCGACTCTGTGAAGGGCCGCTTCACTATCTCCCGGG |
| | | ACAACTCCAAGAACACCCTGTATCTCCAAATGAATTCCCTGAGG |
| | | GCCGAAGATACCGCGGTGTACTACTGCGCTAGACGGGAGTGGT |
| | | GGGGAGAAAGCTGGCTGTTCGACTACTGGGGACAGGGCACTCT |
| | | CGTGACTGTGTCCTCC |
| SEQ ID | LCDR1 | RASQSISSYLN |
| NO: 54 | (Kabat) | |
| SEQ ID | LCDR2 | AASSLQS |
| NO: 55 | (Kabat) | |
| SEQ ID | LCDR3 | QQSYSTPLT |
| NO: 56 | (Kabat) | |
| SEQ ID | LCDR1 | SQSISSY |
| NO: 57 | (Chothia) | |
| SEQ ID | LCDR2 | AAS |
| NO: 58 | (Chothia) | |
| SEQ ID | LCDR3 | SYSTPL |
| NO: 59 | (Chothia) | |
| SEQ ID | LCDR1 | QSISSY |
| NO: 60 | (IMGT) | |
| SEQ ID | LCDR2 | AAS |
| NO: 58 | (IMGT) | |
| SEQ ID | LCDR3 | QQSYSTPLT |
| NO: 56 | (IMGT) | |
| SEQ ID | VL | DIQMTQSPSSLSASVGDRVTITCRASQSISSYLNWYQQKPGKAPKL |
| NO: 61 | | LIYAASSLQSGVPSRFSGSGSGTDFTLTISSLQPEDFATYYCQQSYS |
| | | TPLTFGQGTKVEIK |
| SEQ ID | DNA VL | GACATTCAAATGACTCAGTCCCCGTCCTCCCTCTCCGCCTCCGT |
| NO: 62 | | GGGAGATCGCGTCACGATCACGTGCAGGGCCAGCCAGAGCATC |
| | | TCCAGCTACCTGAACTGGTACCAGCAGAAGCCAGGGAAGGCAC |
| | | CGAAGCTCCTGATCTACGCCGCTAGCTCGCTGCAGTCCGGCGTC |
| | | CCTTCACGGTTCTCGGGATCGGGCTCAGGCACCGACTTCACCCT |
| | | GACCATTAGCAGCCTGCAGCCGGAGGACTTCGCGACATACTAC |
| | | TGTCAGCAGTCATACTCCACCCCTCTGACCTTCGGCCAAGGGAC |
| | | CAAAGTGGAGATCAAG |
| SEQ ID | Linker | GGGGSGGGGSGGGGSGGGGS |
| NO: 63 | | |
| SEQ ID | scFv (VH- | EVQLLESGGGLVQPGGSLRLSCAASGFTFSSYAMSWVRQAPGKGL |
| NO: 80 | linker-VL) | EWVSAISGSGGSTYYADSVKGRFTISRDNSKNTLYLQMNSLRAED |
| | | TAVYYCARREWWGESWLFDYWGQGTLVTVSSGGGGSGGGGSGG |
| | | GGSGGGGSDIQMTQSPSSLSASVGDRVTITCRASQSISSYLNWYQQ |
| | | KPGKAPKLLIYAASSLQSGVPSRFSGSGSGTDFTLTISSLQPEDFAT |
| | | YYCQQSYSTPLTFGQGTKVEIK |
| SEQ ID | DNA scFv | GAAGTGCAGTTGCTGGAGTCAGGCGGAGGACTGGTGCAGCCCG |
| NO: 81 | | GAGGATCGCTTCGCTTGAGCTGCGCAGCCTCAGGCTTTACCTTC |
| | | TCCTCCTACGCCATGTCCTGGGTCAGACAGGCTCCCGGGAAGG |
| | | GACTGGAATGGGTGTCCGCCATTAGCGGTTCCGGCGGAAGCAC |
| | | TTACTATGCCGACTCTGTGAAGGGCCGCTTCACTATCTCCCGGG |
| | | ACAACTCCAAGAACACCCTGTATCTCCAAATGAATTCCCTGAGG |
| | | GCCGAAGATACCGCGGTGTACTACTGCGCTAGACGGGAGTGGT |
| | | GGGGAGAAAGCTGGCTGTTCGACTACTGGGGACAGGGCACTCT |
| | | CGTGACTGTGTCCTCCGGTGGTGGTGGATCGGGGGGTGGTGGTT |
| | | CGGGCGGAGGAGGATCTGGAGGAGGAGGGTCGGACATTCAAA |
| | | TGACTCAGTCCCCGTCCTCCCTCTCCGCCTCCGTGGGAGATCGC |
| | | GTCACGATCACGTGCAGGGCCAGCCAGAGCATCTCCAGCTACC |
| | | TGAACTGGTACCAGCAGAAGCCAGGGAAGGCACCGAAGCTCCT |
| | | GATCTACGCCGCTAGCTCGCTGCAGTCCGGCGTCCCTTCACGGT |
| | | TCTCGGGATCGGGCTCAGGCACCGACTTCACCCTGACCATTAGC |
| | | AGCCTGCAGCCGGAGGACTTCGCGACATACTACTGTCAGCAGT |
| | | CATACTCCACCCCTCTGACCTTCGGCCAAGGGACCAAAGTGGA |
| | | GATCAAG |
| SEQ ID | Full CAR | EVQLLESGGGLVQPGGSLRLSCAASGFTFSSYAMSWVRQAPGKGL |
| NO: 82 | amino acid | EWVSAISGSGGSTYYADSVKGRFTISRDNSKNTLYLQMNSLRAED |
| | sequence | TAVYYCARREWWGESWLFDYWGQGTLVTVSSGGGGSGGGGSGG |
| | | GGSGGGGSDIQMTQSPSSLSASVGDRVTITCRASQSISSYLNWYQQ |
| | | KPGKAPKLLIYAASSLQSGVPSRFSGSGSGTDFTLTISSLQPEDFAT |
| | | YYCQQSYSTPLTFGQGTKVEIKTTTPAPRPPTPAPTIASQPLSLRPEA |
| | | CRPAAGGAVHTRGLDFACDIYIWAPLAGTCGVLLLSLVITLYCKR |
| | | GRKKLLYIFKQPFMRPVQTTQEEDGCSCRFPEEEEGGCELRVKFSR |
| | | SADAPAYQQGQNQLYNELNLGRREEYDVLDKRRGRDPEMGGKP |
| | | RRKNPQEGLYNELQKDKMAEAYSEIGMKGERRRGKGHDGLYQG |
| | | LSTATKDTYDALHMQALPPR |
| SEQ ID | Full CAR | GAAGTGCAGTTGCTGGAGTCAGGCGGAGGACTGGTGCAGCCCG |
| NO: 83 | DNA | GAGGATCGCTTCGCTTGAGCTGCGCAGCCTCAGGCTTTACCTTC |
| | sequence | TCCTCCTACGCCATGTCCTGGGTCAGACAGGCTCCCGGGAAGG |
| | | GACTGGAATGGGTGTCCGCCATTAGCGGTTCCGGCGGAAGCAC |
| | | TTACTATGCCGACTCTGTGAAGGGCCGCTTCACTATCTCCCGGG |
| | | ACAACTCCAAGAACACCCTGTATCTCCAAATGAATTCCCTGAGG |
| | | GCCGAAGATACCGCGGTGTACTACTGCGCTAGACGGGAGTGGT |
| | | GGGGAGAAAGCTGGCTGTTCGACTACTGGGGACAGGGCACTCT |
| | | CGTGACTGTGTCCTCCGGTGGTGGTGGATCGGGGGGTGGTGGTT |
| | | CGGGCGGAGGAGGATCTGGAGGAGGAGGGTCGGACATTCAAA |
| | | TGACTCAGTCCCCGTCCTCCCTCTCCGCCTCCGTGGGAGATCGC |
| | | GTCACGATCACGTGCAGGGCCAGCCAGAGCATCTCCAGCTACC |
| | | TGAACTGGTACCAGCAGAAGCCAGGGAAGGCACCGAAGCTCCT |
| | | GATCTACGCCGCTAGCTCGCTGCAGTCCGGCGTCCCTTCACGGT |
| | | TCTCGGGATCGGGCTCAGGCACCGACTTCACCCTGACCATTAGC |
| | | AGCCTGCAGCCGGAGGACTTCGCGACATACTACTGTCAGCAGT |
| | | CATACTCCACCCCTCTGACCTTCGGCCAAGGGACCAAAGTGGA |
| | | GATCAAGACCACTACCCCAGCACCGAGGCCACCCACCCCGGCT |
| | | CCTACCATCGCCTCCCAGCCTCTGTCCCTGCGTCCGGAGGCATG |
| | | TAGACCCGCAGCTGGTGGGGCCGTGCATACCCGGGGTCTTGAC |
| | | TTCGCCTGCGATATCTACATTTGGGCCCCTCTGGCTGGTACTTG |
| | | CGGGGTCCTGCTGCTTTCACTCGTGATCACTCTTTACTGTAAGC |
| | | GCGGTCGGAAGAAGCTGCTGTACATCTTTAAGCAACCCTTCATG |
| | | AGGCCTGTGCAGACTACTCAAGAGGAGGACGGCTGTTCATGCC |
| | | GGTTCCCAGAGGAGGAGGAAGGCGGCTGCGAACTGCGCGTGAA |
| | | ATTCAGCCGCAGCGCAGATGCTCCAGCCTACCAGCAGGGGCAG |
| | | AACCAGCTCTACAACGAACTCAATCTTGGTCGGAGAGAGGAGT |
| | | ACGACGTGCTGGACAAGCGGAGAGGACGGGACCCAGAAATGG |
| | | GCGGGAAGCCGCGCAGAAAGAATCCCCAAGAGGGCCTGTACA |
| | | ACGAGCTCCAAAAGGATAAGATGGCAGAAGCCTATAGCGAGAT |
| | | TGGTATGAAAGGGGAACGCAGAAGAGGCAAAGGCCACGACGG |
| | | ACTGTACCAGGGACTCAGCACCGCCACCAAGGACACCTATGAC |
| | | GCTCTTCACATGCAGGCCCTGCCGCCTCGG |

TABLE 4
Kabat CDRs of exemplary PALLAS-derived anti-BCMA molecules

| Kabat | HCDR1 | HCDR2 | HCDR3 | LCDR1 | LCDR2 | LCDR3 |
|---|---|---|---|---|---|---|
| R1B6 | SYAMS | AISGSGGSTY | REWVPYDVSWYFDY | RASQSISSYLN | AASSLQS | QQSYSTPLT |
| | (SEQ ID | YADSVKG | (SEQ ID | (SEQ ID | (SEQ ID | (SEQ ID |
| | NO: 44) | (SEQ ID | NO: 46) | NO: 54) | NO: 55) | NO: 56) |
| | | NO: 45) | | | | |
| R1F2 | SYAMS | AISGSGGSTY | REWWYDDWYLDY | RASQSISSYLN | AASSLQS | QQSYSTPLT |
| | (SEQ ID | YADSVKG | (SEQ ID | (SEQ ID | (SEQ ID | (SEQ ID |
| | NO: 44) | (SEQ ID | NO: 68) | NO: 54) | NO: 55) | NO: 56) |
| | | NO: 45) | | | | |
| R1G5 | SYAMS | AISGSGGSTY | REWWGESWLFDY | RASQSISSYLN | AASSLQS | QQSYSTPLT |
| | (SEQ ID | YADSVKG | (SEQ ID | (SEQ ID | (SEQ ID | (SEQ ID |
| | NO: 44) | (SEQ ID | NO: 76) | NO: 54) | NO: 55) | NO: 56) |
| | | NO: 45) | | | | |
| Consensus | SYAMS | AISGSGGSTY | REWX₁X₂X₃X₄ | RASQSISSYLN | AASSLQS | QQSYSTPLT |
| | (SEQ ID | YADSVKG | X₅X₆WX₇X₈DY, | (SEQ ID | (SEQ ID | (SEQ ID |
| | NO: 44) | (SEQ ID | wherein X₁ is | NO: 54) | NO: 55) | NO: 56) |
| | | NO: 45) | absent or V; | | | |
| | | | X₂ is absent or P; | | | |
| | | | X₃ is W or Y; | | | |
| | | | X₄ is G, Y, or D; | | | |
| | | | X₅ is E, D, or V; | | | |
| | | | X₆ is S or D; | | | |
| | | | X₇ is L or Y; and | | | |
| | | | X₈ is F or L | | | |
| | | | (SEQ ID NO: 84) | | | |
| | | | ID NO: 84) | | | |

TABLE 5
Chothia CDRs of exemplary PALLAS-derived anti-BCMA molecules

| Chothia | HCDR1 | HCDR2 | HCDR3 | LCDR1 | LCDR2 | LCDR3 |
|---|---|---|---|---|---|---|
| R1B6 | GFTFSSY | SGSGGS | REWVPYDVS | SQSISSY | AAS | SYSTPL |
| | (SEQ ID | (SEQ ID) | WYFDY (SEQ | (SEQ ID | (SEQ ID | (SEQ ID |
| | NO: 47) | NO: 48 | ID NO: 46) | NO: 57) | NO: 58) | NO: 59) |
| R1F2 | GFTFSSY | SGSGGS | REWWYDD | SQSISSY | AAS | SYSTPL |
| | (SEQ ID | (SEQ ID) | WYLDY (SEQ | (SEQ ID | (SEQ ID | (SEQ ID |
| | NO: 47) | NO: 48 | ID NO: 68) | NO: 57) | NO: 58) | NO: 59) |
| R1G5 | GFTFSSY | SGSGGS | REWWGESW | SQSISSY | AAS | SYSTPL |
| | (SEQ ID | (SEQ ID) | LFDY (SEQ | (SEQ ID | (SEQ ID | (SEQ ID |
| | NO: 47) | NO: 48 | ID NO: 76) | NO: 57) | NO: 58) | NO: 59) |
| Consensus | GFTFSSY | SGSGGS | REWX₁X₂X₃X₄ | SQSISSY | AAS | SYSTPL |
| | (SEQ ID | (SEQ ID) | X₅X₆WX₇X₈DY, | (SEQ ID | (SEQ ID | (SEQ ID |
| | NO: 47) | NO: 48 | wherein X₁ is | NO: 57) | NO: 58) | NO: 59) |
| | | | absent or V; | | | |
| | | | X₂ is absent or P; | | | |
| | | | X₃ is W or Y; | | | |
| | | | X₄ is G, Y, or D; | | | |
| | | | X₅ is E, D, or V; | | | |
| | | | X₆ is S or D; | | | |
| | | | X₇ is L or Y; and | | | |
| | | | X₈ is F or L | | | |
| | | | (SEQ ID NO: 84) | | | |

TABLE 6
IMGT CDRs of exemplary PALLAS-derived anti-BCMA molecules

| IMGT | HCDR1 | HCDR2 | HCDR3 | LCDR1 | LCDR2 | LCDR3 |
|---|---|---|---|---|---|---|
| R1B6 | GFTFSSYA | ISGSGGST | ARREWVPYDV | QSISSY | AAS | QQSYSTP |
| | (SEQ ID | (SEQ ID | SWYFDY | (SEQ ID | (SEQ ID | LT (SEQ |
| | NO: 49) | NO: 50) | (SEQ ID | NO: 60) | NO: 58) | ID NO: |
| | | | NO: 51) | | | 56) |
| R1F2 | GFTFSSYA | ISGSGGST | ARREWWYD | QSISSY | AAS | QQSYSTP |
| | (SEQ ID | (SEQ ID | DWYLDY | (SEQ ID | (SEQ ID | LT (SEQ |
| | NO: 49) | NO: 50) | (SEQ ID NO: | NO: 60) | NO: 58) | ID NO: |
| | | | 69) | | | 56) |
| R1G5 | GFTFSSYA | ISGSGGST | ARREWWGE | QSISSY | AAS | QQSYSTP |
| | (SEQ ID | (SEQ ID | SWLFDY | (SEQ ID | (SEQ ID | LT (SEQ |
| | NO: 49) | NO: 50) | (SEQ ID NO: | NO: 60) | NO: 58) | ID NO: |
| | | | 77) | | | 56) |
| Consensus | GFTFSSYA | ISGSGGST | ARREWX₁X₂X₃X₄X₅X₆WX₇X₈DY, | QSISSY | AAS | QQSYSTP |
| | (SEQ ID | (SEQ ID | wherein | (SEQ ID | (SEQ ID | LT (SEQ |
| | NO: 49) | NO: 50) | X₁ is absent or V; | NO: 60) | NO: 58) | ID NO: |
| | | | X₂ is absent or P; | | | 56) |
| | | | X₃ is W or Y; | | | |
| | | | X₄ is G, Y, or D; | | | |
| | | | X₅ is E, D, or V; | | | |
| | | | X₆ is is S or D; | | | |
| | | | X₇ is L or Y; and | | | |
| | | | X₈ is F or L | | | |
| | | | (SEQ ID NO: 85) | | | |

TABLE 7
Amino acid and nucleic acid sequences of exemplary
B cell-derived anti-BCMA molecules

| | Name/ | |
|---|---|---|
| SEQ ID NO | Description | Sequence |
| PI61 | | |
| SEQ ID | HCDR1 | SYGMH |
| NO: 86 | (Kabat) | |
| SEQ ID | HCDR2 | VISYDGSNKYYADSVKG |
| NO: 87 | (Kabat) | |
| SEQ ID | HCDR3 | SGYALHDDYYGLDV |
| NO: 88 | (Kabat) | |
| SEQ ID | HCDR1 | GFTFSSY |
| NO: 47 | (Chothia) | |
| SEQ ID | HCDR2 | SYDGSN |
| NO: 89 | (Chothia) | |
| SEQ ID | HCDR3 | SGYALHDDYYGLDV |
| NO: 88 | (Chothia) | |
| SEQ ID | HCDR1 | GFTFSSYG |
| NO: 90 | (IMGT) | |
| SEQ ID | HCDR2 | ISYDGSNK |
| NO: 91 | (IMGT) | |
| SEQ ID | HCDR3 | GGSGYALHDDYYGLDV |
| NO: 92 | (IMGT) | |
| SEQ ID | VH | QVQLQESGGGVVQPGRSLRLSCAASGFTFSSYGMHWVRQAPGKGL |
| NO: 93 | | EWVAVISYDGSNKYYADSVKGRFTISRDNSKNTLYLQMNSLRAED |
| | | TAVYYCGGSGYALHDDYYGLDVWGQGTLVTVSS |
| SEQ ID | DNA VH | CAAGTGCAGCTGCAGGAATCCGGTGGCGGAGTCGTGCAGCCTGG |
| NO: 94 | | AAGGAGCCTGAGACTCTCATGCGCCGCGTCAGGGTTCACCTTTT |
| | | CCTCCTACGGGATGCATTGGGTCAGACAGGCCCCCGGAAAGGGA |
| | | CTCGAATGGGTGGCTGTGATCAGCTACGACGGCTCCAACAAGTA |
| | | CTACGCCGACTCCGTGAAAGGCCGGTTCACTATCTCCCGGGACA |
| | | ACTCCAAGAACACGCTGTATCTGCAAATGAATTCACTGCGCGCG |
| | | GAGGATACCGCTGTGTACTACTGCGGTGGCTCCGGTTACGCCCT |
| | | GCACGATGACTATTACGGCCTTGACGTCTGGGGCCAGGGAACCC |
| | | TCGTGACTGTGTCCAGC |
| SEQ ID | LCDR1 | TGTSSDVGGYNYVS |
| NO: 95 | (Kabat) | |
| SEQ ID | LCDR2 | DVSNRPS |
| NO: 96 | (Kabat) | |
| SEQ ID | LCDR3 | SSYTSSSTLYV |
| NO: 97 | (Kabat) | |
| SEQ ID | LCDR1 | TSSDVGGYNY |
| NO: 98 | (Chothia) | |
| SEQ ID | LCDR2 | DVS |
| NO: 99 | (Chothia) | |
| SEQ ID | LCDR3 | YTSSSTLY |
| NO: 100 | (Chothia) | |
| SEQ ID | LCDR1 | SSDVGGYNY |
| NO: 101 | (IMGT) | |
| SEQ ID | LCDR2 | DVS |
| NO: 99 | (IMGT) | |
| SEQ ID | LCDR3 | SSYTSSSTLYV |
| NO: 97 | (IMGT) | |
| SEQ ID | VL | QSALTQPASVSGSPGQSITISCTGTSSDVGGYNYVSWYQQHPGKAP |
| NO: 102 | | KLMIYDVSNRPSGVSNRFSGSKSGNTASLTISGLQAEDEADYYCSSY |
| | | TSSSTLYVFGSGTKVTVL |
| SEQ ID | DNA VL | CAGAGCGCACTGACTCAGCCGGCATCCGTGTCCGGTAGCCCCGG |
| NO: 103 | | ACAGTCGATTACCATCTCCTGTACCGGCACCTCCTCCGACGTGG |
| | | GAGGGTACAACTACGTGTCGTGGTACCAGCAGCACCCAGGAAA |
| | | GGCCCCTAAGTTGATGATCTACGATGTGTCAAACCGCCCGTCTG |
| | | GAGTCTCCAACCGGTTCTCCGGCTCCAAGTCCGGCAACACCGCC |
| | | AGCCTGACCATTAGCGGGCTGCAAGCCGAGGATGAGGCCGACT |
| | | ACTACTGCTCGAGCTACACATCCTCGAGCACCCTCTACGTGTTCG |
| | | GCTCGGGGACTAAGGTCACCGTGCTG |
| SEQ ID | Linker | GGGGSGGGGSGGGGS |
| NO: 104 | | |
| SEQ ID | scFv (VH- | QVQLQESGGGVVQPGRSLRLSCAASGFTFSSYGMHWVRQAPGKGL |
| NO: 105 | linker-VL) | EWVAVISYDGSNKYYADSVKGRFTISRDNSKNTLYLQMNSLRAED |
| | | TAVYYCGGSGYALHDDYYGLDVWGQGTLVTVSSGGGGSGGGGS |
| | | GGGGSQSALTQPASVSGSPGQSITISCTGTSSDVGGYNYVSWYQQH |
| | | PGKAPKLMIYDVSNRPSGVSNRFSGSKSGNTASLTISGLQAEDEAD |
| | | YYCSSYTSSSTLYVFGSGTKVTVL |
| SEQ ID | DNA scFv | CAAGTGCAGCTGCAGGAATCCGGTGGCGGAGTCGTGCAGCCTGG |
| NO: 106 | | AAGGAGCCTGAGACTCTCATGCGCCGCGTCAGGGTTCACCTTTT |
| | | CCTCCTACGGGATGCATTGGGTCAGACAGGCCCCCGGAAAGGGA |
| | | CTCGAATGGGTGGCTGTGATCAGCTACGACGGCTCCAACAAGTA |
| | | CTACGCCGACTCCGTGAAAGGCCGGTTCACTATCTCCCGGGACA |
| | | ACTCCAAGAACACGCTGTATCTGCAAATGAATTCACTGCGCGCG |
| | | GAGGATACCGCTGTGTACTACTGCGGTGGCTCCGGTTACGCCCT |
| | | GCACGATGACTATTACGGCCTTGACGTCTGGGGCCAGGGAACCC |
| | | TCGTGACTGTGTCCAGCGGTGGAGGAGGTTCGGGCGGAGGAGG |
| | | ATCAGGAGGGGGTGGATCGCAGAGCGCACTGACTCAGCCGGCA |
| | | TCCGTGTCCGGTAGCCCCGGACAGTCGATTACCATCTCCTGTACC |
| | | GGCACCTCCTCCGACGTGGGAGGGTACAACTACGTGTCGTGGTA |
| | | CCAGCAGCACCCAGGAAAGGCCCCTAAGTTGATGATCTACGATG |
| | | TGTCAAACCGCCCGTCTGGAGTCTCCAACCGGTTCTCCGGCTCCA |
| | | AGTCCGGCAACACCGCCAGCCTGACCATTAGCGGGCTGCAAGCC |
| | | GAGGATGAGGCCGACTACTACTGCTCGAGCTACACATCCTCGAG |
| | | CACCCTCTACGTGTTCGGCTCGGGGACTAAGGTCACCGTGCTG |
| SEQ ID | Full CAR | QVQLQESGGGVVQPGRSLRLSCAASGFTFSSYGMHWVRQAPGKGL |
| NO: 107 | amino acid | EWVAVISYDGSNKYYADSVKGRFTISRDNSKNTLYLQMNSLRAED |
| | sequence | TAVYYCGGSGYALHDDYYGLDVWGQGTLVTVSSGGGGSGGGGS |
| | | GGGGSQSALTQPASVSGSPGQSITISCTGTSSDVGGYNYVSWYQQH |
| | | PGKAPKLMIYDVSNRPSGVSNRFSGSKSGNTASLTISGLQAEDEAD |
| | | YYCSSYTSSSTLYVFGSGTKVTVLTTTPAPRPPTPAPTIASQPLSLRP |
| | | EACRPAAGGAVHTRGLDFACDIYIWAPLAGTCGVLLLSLVITLYCK |
| | | RGRKKLLYIFKQPFMRPVQTTQEEDGCSCRFPEEEEGGCELRVKFSR |
| | | SADAPAYQQGQNQLYNELNLGRREEYDVLDKRRGRDPEMGGKPR |
| | | RKNPQEGLYNELQKDKMAEAYSEIGMKGERRRGKGHDGLYQGLS |
| | | TATKDTYDALHMQALPPR |
| SEQ ID | Full CAR | CAAGTGCAGCTGCAGGAATCCGGTGGCGGAGTCGTGCAGCCTGG |
| NO: 108 | DNA | AAGGAGCCTGAGACTCTCATGCGCCGCGTCAGGGTTCACCTTTT |
| | sequence | CCTCCTACGGGATGCATTGGGTCAGACAGGCCCCCGGAAAGGGA |
| | | CTCGAATGGGTGGCTGTGATCAGCTACGACGGCTCCAACAAGTA |
| | | CTACGCCGACTCCGTGAAAGGCCGGTTCACTATCTCCCGGGACA |
| | | ACTCCAAGAACACGCTGTATCTGCAAATGAATTCACTGCGCGCG |
| | | GAGGATACCGCTGTGTACTACTGCGGTGGCTCCGGTTACGCCCT |
| | | GCACGATGACTATTACGGCCTTGACGTCTGGGGCCAGGGAACCC |
| | | TCGTGACTGTGTCCAGCGGTGGAGGAGGTTCGGGCGGAGGAGG |
| | | ATCAGGAGGGGGTGGATCGCAGAGCGCACTGACTCAGCCGGCA |
| | | TCCGTGTCCGGTAGCCCCGGACAGTCGATTACCATCTCCTGTACC |
| | | GGCACCTCCTCCGACGTGGGAGGGTACAACTACGTGTCGTGGTA |
| | | CCAGCAGCACCCAGGAAAGGCCCCTAAGTTGATGATCTACGATG |
| | | TGTCAAACCGCCCGTCTGGAGTCTCCAACCGGTTCTCCGGCTCCA |
| | | AGTCCGGCAACACCGCCAGCCTGACCATTAGCGGGCTGCAAGCC |
| | | GAGGATGAGGCCGACTACTACTGCTCGAGCTACACATCCTCGAG |
| | | CACCCTCTACGTGTTCGGCTCGGGGACTAAGGTCACCGTGCTGA |
| | | CCACTACCCCAGCACCGAGGCCACCCACCCCGGCTCCTACCATC |
| | | GCCTCCCAGCCTCTGTCCCTGCGTCCGGAGGCATGTAGACCCGC |
| | | AGCTGGTGGGGCCGTGCATACCCGGGGTCTTGACTTCGCCTGCG |
| | | ATATCTACATTTGGGCCCCTCTGGCTGGTACTTGCGGGGTCCTGC |
| | | TGCTTTCACTCGTGATCACTCTTTACTGTAAGCGCGGTCGGAAGA |
| | | AGCTGCTGTACATCTTTAAGCAACCCTTCATGAGGCCTGTGCAG |
| | | ACTACTCAAGAGGAGGACGGCTGTTCATGCCGGTTCCCAGAGGA |
| | | GGAGGAAGGCGGCTGCGAACTGCGCGTGAAATTCAGCCGCAGC |
| | | GCAGATGCTCCAGCCTACCAGCAGGGGCAGAACCAGCTCTACAA |
| | | CGAACTCAATCTTGGTCGGAGAGAGGAGTACGACGTGCTGGACA |
| | | AGCGGAGAGGACGGGACCCAGAAATGGGCGGGAAGCCGCGCAG |
| | | AAAGAATCCCCAAGAGGGCCTGTACAACGAGCTCCAAAAGGAT |
| | | AAGATGGCAGAAGCCTATAGCGAGATTGGTATGAAAGGGGAAC |
| | | GCAGAAGAGGCAAAGGCCACGACGGACTGTACCAGGGACTCAG |
| | | CACCGCCACCAAGGACACCTATGACGCTCTTCACATGCAGGCCC |
| | | TGCCGCCTCGG |
| B61-02 | | |
| SEQ ID | HCDR1 | SYGMH |
| NO: 86 | (Kabat) | |
| SEQ ID | HCDR2 | VISYKGSNKYYADSVKG |
| NO: 109 | (Kabat) | |
| SEQ ID | HCDR3 | SGYALHDDYYGLDV |
| NO: 88 | (Kabat) | |
| SEQ ID | HCDR1 | GFTFSSY |
| NO: 47 | (Chothia) | |
| SEQ ID | HCDR2 | SYKGSN |
| NO: 110 | (Chothia) | |
| SEQ ID | HCDR3 | SGYALHDDYYGLDV |
| NO: 88 | (Chothia) | |
| SEQ ID | HCDR1 | GFTFSSYG |
| NO: 90 | (IMGT) | |
| SEQ ID | HCDR2 | ISYKGSNK |
| NO: 111 | (IMGT) | |
| SEQ ID | HCDR3 | GGSGYALHDDYYGLDV |
| NO: 92 | (IMGT) | |
| SEQ ID | VH | QVQLVESGGGVVQPGRSLRLSCAASGFTFSSYGMHWVRQAPGKGL |
| NO: 112 | | EWVAVISYKGSNKYYADSVKGRFTISRDNSKNTLYLQMNSLRAED |
| | | TAVYYCGGSGYALHDDYYGLDVWGQGTLVTVSS |
| SEQ ID | DNA VH | CAAGTGCAGCTTGTCGAATCGGGAGGCGGAGTGGTGCAGCCTGG |
| NO: 113 | | ACGATCGCTCCGGCTCTCATGTGCCGCGAGCGGATTCACCTTCTC |
| | | GAGCTACGGCATGCACTGGGTCAGACAAGCCCCAGGAAAGGGC |
| | | CTGGAATGGGTGGCTGTCATCTCGTACAAGGGCTCAAACAAGTA |
| | | CTACGCCGACTCCGTGAAGGGCCGGTTCACCATCTCCCGCGATA |
| | | ACTCCAAGAATACCCTCTATCTGCAAATGAACAGCCTGAGGGCC |
| | | GAGGATACTGCAGTGTACTACTGCGGGGGTTCAGGCTACGCGCT |
| | | GCACGACGACTACTACGGATTGGACGTCTGGGGCCAAGGAACTC |
| | | TTGTGACCGTGTCCTCT |
| SEQ ID | LCDR1 | TGTSSDVGGYNYVS |
| NO: 95 | (Kabat) | |
| SEQ ID | LCDR2 | EVSNRLR |
| NO: 114 | (Kabat) | |
| SEQ ID | LCDR3 | SSYTSSSALYV |
| NO: 115 | (Kabat) | |
| SEQ ID | LCDR1 | TSSDVGGYNY |
| NO: 98 | (Chothia) | |
| SEQ ID | LCDR2 | EVS |
| NO: 116 | (Chothia) | |
| SEQ ID | LCDR3 | YTSSSALY |
| NO: 117 | (Chothia) | |
| SEQ ID | LCDR1 | SSDVGGYNY |
| NO: 101 | (IMGT) | |
| SEQ ID | LCDR2 | EVS |
| NO: 116 | (IMGT) | |
| SEQ ID | LCDR3 | SSYTSSSALYV |
| NO: 115 | (IMGT) | |
| SEQ ID | VL | QSALTQPASVSGSPGQSITISCTGTSSDVGGYNYVSWYQQHPGKAP |
| NO: 118 | | KLMIYEVSNRLRGVSNRFSGSKSGNTASLTISGLQAEDEADYYCSS |
| | | YTSSSALYVFGSGTKVTVL |
| SEQ ID | DNA VL | CAGAGCGCGCTGACTCAGCCTGCCTCCGTGAGCGGTTCGCCGGG |
| NO: 119 | | ACAGTCCATTACCATTTCGTGCACCGGGACCTCCTCCGACGTGG |
| | | GAGGCTACAACTACGTGTCCTGGTACCAGCAGCATCCCGGAAAG |
| | | GCCCCGAAGCTGATGATCTACGAAGTGTCGAACAGACTGCGGGG |
| | | AGTCTCCAACCGCTTTTCCGGGTCCAAGTCCGGCAACACCGCCA |
| | | GCCTGACCATCAGCGGGCTCCAGGCAGAAGATGAGGCTGACTAT |
| | | TACTGCTCCTCCTACACGTCAAGCTCCGCCCTCTACGTGTTCGGG |
| | | TCCGGGACCAAAGTCACTGTGCTG |
| SEQ ID | Linker | GGGGSGGGGSGGGGSGGGGS |
| NO: 63 | | |
| SEQ ID | scFv (VH- | QVQLVESGGGVVQPGRSLRLSCAASGFTFSSYGMHWVRQAPGKGL |
| NO: 120 | linker-VL) | EWVAVISYKGSNKYYADSVKGRFTISRDNSKNTLYLQMNSLRAED |
| | | TAVYYCGGSGYALHDDYYGLDVWGQGTLVTVSSGGGGSGGGGS |
| | | GGGGSGGGGSQSALTQPASVSGSPGQSITISCTGTSSDVGGYNYVS |
| | | WYQQHPGKAPKLMIYEVSNRLRGVSNRFSGSKSGNTASLTISGLQA |
| | | EDEADYYCSSYTSSSALYVFGSGTKVTVL |
| SEQ ID | DNA scFv | CAAGTGCAGCTTGTCGAATCGGGAGGCGGAGTGGTGCAGCCTGG |
| NO: 121 | | ACGATCGCTCCGGCTCTCATGTGCCGCGAGCGGATTCACCTTCTC |
| | | GAGCTACGGCATGCACTGGGTCAGACAAGCCCCAGGAAAGGGC |
| | | CTGGAATGGGTGGCTGTCATCTCGTACAAGGGCTCAAACAAGTA |
| | | CTACGCCGACTCCGTGAAGGGCCGGTTCACCATCTCCCGCGATA |
| | | ACTCCAAGAATACCCTCTATCTGCAAATGAACAGCCTGAGGGCC |
| | | GAGGATACTGCAGTGTACTACTGCGGGGGTTCAGGCTACGCGCT |
| | | GCACGACGACTACTACGGATTGGACGTCTGGGGCCAAGGAACTC |
| | | TTGTGACCGTGTCCTCTGGTGGAGGCGGATCAGGGGGTGGCGGA |
| | | TCTGGGGGTGGTGGTTCCGGGGGAGGAGGATCGCAGAGCGCGC |
| | | TGACTCAGCCTGCCTCCGTGAGCGGTTCGCCGGGACAGTCCATT |
| | | ACCATTTCGTGCACCGGGACCTCCTCCGACGTGGGAGGCTACAA |
| | | CTACGTGTCCTGGTACCAGCAGCATCCCGGAAAGGCCCCGAAGC |
| | | TGATGATCTACGAAGTGTCGAACAGACTGCGGGGAGTCTCCAAC |
| | | CGCTTTTCCGGGTCCAAGTCCGGCAACACCGCCAGCCTGACCAT |
| | | CAGCGGGCTCCAGGCAGAAGATGAGGCTGACTATTACTGCTCCT |
| | | CCTACACGTCAAGCTCCGCCCTCTACGTGTTCGGGTCCGGGACC |
| | | AAAGTCACTGTGCTG |
| SEQ ID | Full CAR | QVQLVESGGGVVQPGRSLRLSCAASGFTFSSYGMHWVRQAPGKGL |
| NO: 122 | amino acid | EWVAVISYKGSNKYYADSVKGRFTISRDNSKNTLYLQMNSLRAED |
| | sequence | TAVYYCGGSGYALHDDYYGLDVWGQGTLVTVSSGGGGSGGGGS |
| | | GGGGSGGGGSQSALTQPASVSGSPGQSITISCTGTSSDVGGYNYVS |
| | | WYQQHPGKAPKLMIYEVSNRLRGVSNRFSGSKSGNTASLTISGLQA |
| | | EDEADYYCSSYTSSSALYVFGSGTKVTVLTTTPAPRPPTPAPTIASQP |
| | | LSLRPEACRPAAGGAVHTRGLDFACDIYIWAPLAGTCGVLLLSLVIT |
| | | LYCKRGRKKLLYIFKQPFMRPVQTTQEEDGCSCRFPEEEEGGCELR |
| | | VKFSRSADAPAYQQGQNQLYNELNLGRREEYDVLDKRRGRDPEM |
| | | GGKPRRKNPQEGLYNELQKDKMAEAYSEIGMKGERRRGKGHDGL |
| | | YQGLSTATKDTYDALHMQALPPR |
| SEQ ID | Full CAR | CAAGTGCAGCTTGTCGAATCGGGAGGCGGAGTGGTGCAGCCTGG |
| NO: 123 | DNA | ACGATCGCTCCGGCTCTCATGTGCCGCGAGCGGATTCACCTTCTC |
| | sequence | GAGCTACGGCATGCACTGGGTCAGACAAGCCCCAGGAAAGGGC |
| | | CTGGAATGGGTGGCTGTCATCTCGTACAAGGGCTCAAACAAGTA |
| | | CTACGCCGACTCCGTGAAGGGCCGGTTCACCATCTCCCGCGATA |
| | | ACTCCAAGAATACCCTCTATCTGCAAATGAACAGCCTGAGGGCC |
| | | GAGGATACTGCAGTGTACTACTGCGGGGGTTCAGGCTACGCGCT |
| | | GCACGACGACTACTACGGATTGGACGTCTGGGGCCAAGGAACTC |
| | | TTGTGACCGTGTCCTCTGGTGGAGGCGGATCAGGGGGTGGCGGA |
| | | TCTGGGGGTGGTGGTTCCGGGGGAGGAGGATCGCAGAGCGCGC |
| | | TGACTCAGCCTGCCTCCGTGAGCGGTTCGCCGGGACAGTCCATT |
| | | ACCATTTCGTGCACCGGGACCTCCTCCGACGTGGGAGGCTACAA |
| | | CTACGTGTCCTGGTACCAGCAGCATCCCGGAAAGGCCCCGAAGC |
| | | TGATGATCTACGAAGTGTCGAACAGACTGCGGGGAGTCTCCAAC |
| | | CGCTTTTCCGGGTCCAAGTCCGGCAACACCGCCAGCCTGACCAT |
| | | CAGCGGGCTCCAGGCAGAAGATGAGGCTGACTATTACTGCTCCT |
| | | CCTACACGTCAAGCTCCGCCCTCTACGTGTTCGGGTCCGGGACC |
| | | AAAGTCACTGTGCTGACCACTACCCCAGCACCGAGGCCACCCAC |
| | | CCCGGCTCCTACCATCGCCTCCCAGCCTCTGTCCCTGCGTCCGGA |
| | | GGCATGTAGACCCGCAGCTGGTGGGGCCGTGCATACCCGGGGTC |
| | | TTGACTTCGCCTGCGATATCTACATTTGGGCCCCTCTGGCTGGTA |
| | | CTTGCGGGGTCCTGCTGCTTTCACTCGTGATCACTCTTTACTGTA |
| | | AGCGCGGTCGGAAGAAGCTGCTGTACATCTTTAAGCAACCCTTC |
| | | ATGAGGCCTGTGCAGACTACTCAAGAGGAGGACGGCTGTTCATG |
| | | CCGGTTCCCAGAGGAGGAGGAAGGCGGCTGCGAACTGCGCGTG |
| | | AAATTCAGCCGCAGCGCAGATGCTCCAGCCTACCAGCAGGGGCA |
| | | GAACCAGCTCTACAACGAACTCAATCTTGGTCGGAGAGAGGAGT |
| | | ACGACGTGCTGGACAAGCGGAGAGGACGGGACCCAGAAATGGG |
| | | CGGGAAGCCGCGCAGAAAGAATCCCCAAGAGGGCCTGTACAAC |
| | | GAGCTCCAAAAGGATAAGATGGCAGAAGCCTATAGCGAGATTG |
| | | GTATGAAAGGGGAACGCAGAAGAGGCAAAGGCCACGACGGACT |
| | | GTACCAGGGACTCAGCACCGCCACCAAGGACACCTATGACGCTC |
| | | TTCACATGCAGGCCCTGCCGCCTCGG |
| B61-10 | | |
| SEQ ID | HCDR1 | SYGMH |
| NO: 86 | (Kabat) | |
| SEQ ID | HCDR2 | VISYKGSNKYYADSVKG |
| NO: 109 | (Kabat) | |
| SEQ ID | HCDR3 | SGYALHDDYYGLDV |
| NO: 88 | (Kabat) | |
| SEQ ID | HCDR1 | GFTFSSY |
| NO: 47 | (Chothia) | |
| SEQ ID | HCDR2 | SYKGSN |
| NO: 110 | (Chothia) | |
| SEQ ID | HCDR3 | SGYALHDDYYGLDV |
| NO: 88 | (Chothia) | |
| SEQ ID | HCDR1 | GFTFSSYG |
| NO: 90 | (IMGT) | |
| SEQ ID | HCDR2 | ISYKGSNK |
| NO: 111 | (IMGT) | |
| SEQ ID | HCDR3 | GGSGYALHDDYYGLDV |
| NO: 92 | (IMGT) | |
| SEQ ID | VH | QVQLVESGGGVVQPGRSLRLSCAASGFTFSSYGMHWVRQAPGKGL |
| NO: 112 | | EWVAVISYKGSNKYYADSVKGRFTISRDNSKNTLYLQMNSLRAED |
| | | TAVYYCGGSGYALHDDYYGLDVWGQGTLVTVSS |
| SEQ ID | DNA VH | CAAGTGCAGCTTGTCGAATCGGGAGGCGGAGTGGTGCAGCCTGG |
| NO: 113 | | ACGATCGCTCCGGCTCTCATGTGCCGCGAGCGGATTCACCTTCTC |
| | | GAGCTACGGCATGCACTGGGTCAGACAAGCCCCAGGAAAGGGC |
| | | CTGGAATGGGTGGCTGTCATCTCGTACAAGGGCTCAAACAAGTA |
| | | CTACGCCGACTCCGTGAAGGGCCGGTTCACCATCTCCCGCGATA |
| | | ACTCCAAGAATACCCTCTATCTGCAAATGAACAGCCTGAGGGCC |
| | | GAGGATACTGCAGTGTACTACTGCGGGGGTTCAGGCTACGCGCT |
| | | GCACGACGACTACTACGGATTGGACGTCTGGGGCCAAGGAACTC |
| | | TTGTGACCGTGTCCTCT |
| SEQ ID | LCDR1 | TGTSSDVGGYNYVS |
| NO: 95 | (Kabat) | |
| SEQ ID | LCDR2 | EVSNRLR |
| NO: 114 | (Kabat) | |
| SEQ ID | LCDR3 | SSYTSSSTLYV |
| NO: 97 | (Kabat) | |
| SEQ ID | LCDR1 | TSSDVGGYNY |
| NO: 98 | (Chothia) | |
| SEQ ID | LCDR2 | EVS |
| NO: 116 | (Chothia) | |
| SEQ ID | LCDR3 | YTSSSTLY |
| NO: 100 | (Chothia) | |
| SEQ ID | LCDR1 | SSDVGGYNY |
| NO: 101 | (IMGT) | |
| SEQ ID | LCDR2 | EVS |
| NO: 116 | (IMGT) | |
| SEQ ID | LCDR3 | SSYTSSSTLYV |
| NO: 97 | (IMGT) | |
| SEQ ID | VL | QSALTQPASVSGSPGQSITISCTGTSSDVGGYNYVSWYQQHPGKAP |
| NO: 124 | | KLMIYEVSNRLRGVSNRFSGSKSGNTASLTISGLQAEDEADYYCSS |
| | | YTSSSTLYVFGSGTKVTVL |
| SEQ ID | DNA VL | CAGAGCGCGCTGACTCAGCCTGCCTCCGTGAGCGGTTCGCCGGG |
| NO: 125 | | ACAGTCCATTACCATTTCGTGCACCGGGACCTCCTCCGACGTGG |
| | | GAGGCTACAACTACGTGTCCTGGTACCAGCAGCATCCCGGAAAG |
| | | GCCCCGAAGCTGATGATCTACGAAGTGTCGAACAGACTGCGGGG |
| | | AGTCTCCAACCGCTTTTCCGGGTCCAAGTCCGGCAACACCGCCA |
| | | GCCTGACCATCAGCGGGCTCCAGGCAGAAGATGAGGCTGACTAT |
| | | TACTGCTCCTCCTACACGTCAAGCTCCACCCTCTACGTGTTCGGG |
| | | TCCGGGACCAAAGTCACTGTGCTG |
| SEQ ID | Linker | GGGGSGGGGSGGGGSGGGGS |
| NO: 63 | | |
| SEQ ID | scFv (VH- | QVQLVESGGGVVQPGRSLRLSCAASGFTFSSYGMHWVRQAPGKGL |
| NO: 126 | linker-VL) | EWVAVISYKGSNKYYADSVKGRFTISRDNSKNTLYLQMNSLRAED |
| | | TAVYYCGGSGYALHDDYYGLDVWGQGTLVTVSSGGGGSGGGGS |
| | | GGGGSGGGGSQSALTQPASVSGSPGQSITISCTGTSSDVGGYNYVS |
| | | WYQQHPGKAPKLMIYEVSNRLRGVSNRFSGSKSGNTASLTISGLQA |
| | | EDEADYYCSSYTSSSTLYVFGSGTKVTVL |
| SEQ ID | DNA scFv | CAAGTGCAGCTTGTCGAATCGGGAGGCGGAGTGGTGCAGCCTGG |
| NO: 127 | | ACGATCGCTCCGGCTCTCATGTGCCGCGAGCGGATTCACCTTCTC |
| | | GAGCTACGGCATGCACTGGGTCAGACAAGCCCCAGGAAAGGGC |
| | | CTGGAATGGGTGGCTGTCATCTCGTACAAGGGCTCAAACAAGTA |
| | | CTACGCCGACTCCGTGAAGGGCCGGTTCACCATCTCCCGCGATA |
| | | ACTCCAAGAATACCCTCTATCTGCAAATGAACAGCCTGAGGGCC |
| | | GAGGATACTGCAGTGTACTACTGCGGGGGTTCAGGCTACGCGCT |
| | | GCACGACGACTACTACGGATTGGACGTCTGGGGCCAAGGAACTC |
| | | TTGTGACCGTGTCCTCTGGTGGAGGCGGATCAGGGGGTGGCGGA |
| | | TCTGGGGGTGGTGGTTCCGGGGGAGGAGGATCGCAGAGCGCGC |
| | | TGACTCAGCCTGCCTCCGTGAGCGGTTCGCCGGGACAGTCCATT |
| | | ACCATTTCGTGCACCGGGACCTCCTCCGACGTGGGAGGCTACAA |
| | | CTACGTGTCCTGGTACCAGCAGCATCCCGGAAAGGCCCCGAAGC |
| | | TGATGATCTACGAAGTGTCGAACAGACTGCGGGGAGTCTCCAAC |
| | | CGCTTTTCCGGGTCCAAGTCCGGCAACACCGCCAGCCTGACCAT |
| | | CAGCGGGCTCCAGGCAGAAGATGAGGCTGACTATTACTGCTCCT |
| | | CCTACACGTCAAGCTCCACCCTCTACGTGTTCGGGTCCGGGACC |
| | | AAAGTCACTGTGCTG |
| SEQ ID | Full CAR | QVQLVESGGGVVQPGRSLRLSCAASGFTFSSYGMHWVRQAPGKGL |
| NO: 128 | amino acid | EWVAVISYKGSNKYYADSVKGRFTISRDNSKNTLYLQMNSLRAED |
| | sequence | TAVYYCGGSGYALHDDYYGLDVWGQGTLVTVSSGGGGSGGGGS |
| | | GGGGSGGGGSQSALTQPASVSGSPGQSITISCTGTSSDVGGYNYVS |
| | | WYQQHPGKAPKLMIYEVSNRLRGVSNRFSGSKSGNTASLTISGLQA |
| | | EDEADYYCSSYTSSSTLYVFGSGTKVTVLTTTPAPRPPTPAPTIASQP |
| | | LSLRPEACRPAAGGAVHTRGLDFACDIYIWAPLAGTCGVLLLSLVIT |
| | | LYCKRGRKKLLYIFKQPFMRPVQTTQEEDGCSCRFPEEEEGGCELR |
| | | VKFSRSADAPAYQQGQNQLYNELNLGRREEYDVLDKRRGRDPEM |
| | | GGKPRRKNPQEGLYNELQKDKMAEAYSEIGMKGERRRGKGHDGL |
| | | YQGLSTATKDTYDALHMQALPPR |
| SEQ ID | Full CAR | CAAGTGCAGCTTGTCGAATCGGGAGGCGGAGTGGTGCAGCCTGG |
| NO: 129 | DNA | ACGATCGCTCCGGCTCTCATGTGCCGCGAGCGGATTCACCTTCTC |
| | sequence | GAGCTACGGCATGCACTGGGTCAGACAAGCCCCAGGAAAGGGC |
| | | CTGGAATGGGTGGCTGTCATCTCGTACAAGGGCTCAAACAAGTA |
| | | CTACGCCGACTCCGTGAAGGGCCGGTTCACCATCTCCCGCGATA |
| | | ACTCCAAGAATACCCTCTATCTGCAAATGAACAGCCTGAGGGCC |
| | | GAGGATACTGCAGTGTACTACTGCGGGGGTTCAGGCTACGCGCT |
| | | GCACGACGACTACTACGGATTGGACGTCTGGGGCCAAGGAACTC |
| | | TTGTGACCGTGTCCTCTGGTGGAGGCGGATCAGGGGGTGGCGGA |
| | | TCTGGGGGTGGTGGTTCCGGGGGAGGAGGATCGCAGAGCGCGC |
| | | TGACTCAGCCTGCCTCCGTGAGCGGTTCGCCGGGACAGTCCATT |
| | | ACCATTTCGTGCACCGGGACCTCCTCCGACGTGGGAGGCTACAA |
| | | CTACGTGTCCTGGTACCAGCAGCATCCCGGAAAGGCCCCGAAGC |
| | | TGATGATCTACGAAGTGTCGAACAGACTGCGGGGAGTCTCCAAC |
| | | CGCTTTTCCGGGTCCAAGTCCGGCAACACCGCCAGCCTGACCAT |
| | | CAGCGGGCTCCAGGCAGAAGATGAGGCTGACTATTACTGCTCCT |
| | | CCTACACGTCAAGCTCCACCCTCTACGTGTTCGGGTCCGGGACC |
| | | AAAGTCACTGTGCTGACCACTACCCCAGCACCGAGGCCACCCAC |
| | | CCCGGCTCCTACCATCGCCTCCCAGCCTCTGTCCCTGCGTCCGGA |
| | | GGCATGTAGACCCGCAGCTGGTGGGGCCGTGCATACCCGGGGTC |
| | | TTGACTTCGCCTGCGATATCTACATTTGGGCCCCTCTGGCTGGTA |
| | | CTTGCGGGGTCCTGCTGCTTTCACTCGTGATCACTCTTTACTGTA |
| | | AGCGCGGTCGGAAGAAGCTGCTGTACATCTTTAAGCAACCCTTC |
| | | ATGAGGCCTGTGCAGACTACTCAAGAGGAGGACGGCTGTTCATG |
| | | CCGGTTCCCAGAGGAGGAGGAAGGCGGCTGCGAACTGCGCGTG |
| | | AAATTCAGCCGCAGCGCAGATGCTCCAGCCTACCAGCAGGGGCA |
| | | GAACCAGCTCTACAACGAACTCAATCTTGGTCGGAGAGAGGAGT |
| | | ACGACGTGCTGGACAAGCGGAGAGGACGGGACCCAGAAATGGG |
| | | CGGGAAGCCGCGCAGAAAGAATCCCCAAGAGGGCCTGTACAAC |
| | | GAGCTCCAAAAGGATAAGATGGCAGAAGCCTATAGCGAGATTG |
| | | GTATGAAAGGGGAACGCAGAAGAGGCAAAGGCCACGACGGACT |
| | | GTACCAGGGACTCAGCACCGCCACCAAGGACACCTATGACGCTC |
| | | TTCACATGCAGGCCCTGCCGCCTCGG |

TABLE 8
Kabat CDRs of exemplary B cell-derived anti-BCMA molecules

| Kabat | HCDR1 | HCDR2 | HCDR3 | LCDR1 | LCDR2 | LCDR3 |
|---|---|---|---|---|---|---|
| PI61 | SYGMH | VISYDGSNKYY | SGYALHDD | TGTSSDVGGY | DVSNRPS | SSYTSSSTL |
| | (SEQ ID | ADSVKG | YYGLDV | NYVS | (SEQ ID NO: 96) | YV |
| | NO: 86) | (SEQ ID | (SEQ ID | (SEQ ID | | (SEQ ID |
| | | NO: 87) | NO: 88) | NO: 95) | | NO: 97) |
| B61-02 | SYGMH | VISYKGSNKYY | SGYALHDD | TGTSSDVGGY | EVSNRLR | SSYTSSSAL |
| | (SEQ ID | ADSVKG | YYGLDV | NYVS | (SEQ ID NO: 114) | YV |
| | NO: 86) | (SEQ ID | (SEQ ID | (SEQ ID | | (SEQ ID |
| | | NO: 109) | NO: 88) | NO: 95) | | NO: 115) |
| B61-10 | SYGMH | VISYKGSNKYY | SGYALHDD | TGTSSDVGGY | EVSNRLR | SSYTSSSTL |
| | (SEQ ID | ADSVKG | YYGLDV | NYVS | (SEQ ID NO: 114) | YV |
| | NO: 86) | (SEQ ID | (SEQ ID | (SEQ ID | | (SEQ ID |
| | | NO: 109) | NO: 88) | NO: 95) | | NO: 97) |
| Consensus | SYGMH | VISYXGSNKYY | SGYALHDD | TGTSSDVGGY | X₁VSNRX₂X₃, | SSYTSSSXL |
| | (SEQ ID | ADSVKG, | YYGLDV | NYVS | wherein X₁ is | YV, |
| | NO: 86) | wherein X | (SEQ ID | (SEQ ID | D or E; X₂ is P | wherein X |
| | | is D or K | NO: 88) | NO: 95) | or L; and X₃ is | is T or A |
| | | (SEQ ID | | | S or R | (SEQ ID |
| | | NO: 130) | | | (SEQ ID NO: 131) | NO: 132) |

TABLE 9
Chothia CDRs of exemplary B cell-derived anti-BCMA molecules

| Chothia | HCDR1 | HCDR2 | HCDR3 | LCDR1 | LCDR2 | LCDR3 |
|---|---|---|---|---|---|---|
| PI61 | GFTFSSY | SYDGSN | SGYALHDDY | TSSDVGG | DVS | YTSSSTLY |
| | (SEQ ID | (SEQ ID | YGLDV (SEQ | YNY (SEQ | (SEQ ID | (SEQ ID |
| | NO: 47) | NO: 89) | ID NO: 88) | ID NO: 98) | NO: 99) | NO: 100) |
| B61-02 | GFTFSSY | SYKGSN | SGYALHDDY | TSSDVGG | EVS | YTSSSALY |
| | (SEQ ID | (SEQ ID | YGLDV (SEQ | YNY (SEQ | (SEQ ID | (SEQ ID |
| | NO: 47) | NO: 110) | ID NO: 88) | ID NO: 98) | NO: 116) | NO: 117) |
| B61-10 | GFTFSSY | SYKGSN | SGYALHDDY | TSSDVGG | EVS | YTSSSTLY |
| | (SEQ ID | (SEQ ID | YGLDV (SEQ | YNY (SEQ | (SEQ ID | (SEQ ID |
| | NO: 47) | NO: 110) | ID NO: 88) | ID NO: 98) | NO: 116) | NO: 100) |
| Consensus | GFTFSSY | SYXGSN, | SGYALHDDY | TSSDVGG | XVS, | YTSSSXLY, |
| | (SEQ ID | wherein X | YGLDV (SEQ | YNY (SEQ | wherein X | wherein X |
| | NO: 47) | is D or K | ID NO: 88) | ID NO: 98) | is D or E | is T or A |
| | | (SEQ ID | | | (SEQ ID | (SEQ ID |
| | | NO: 133) | | | NO: 134) | NO: 135) |

TABLE 10
IMGT CDRs of exemplary B cell-derived anti-BCMA molecules

| IMGT | HCDR1 | HCDR2 | HCDR3 | LCDR1 | LCDR2 | LCDR3 |
|---|---|---|---|---|---|---|
| PI61 | GFTFSSYG | ISYDGSNK | GGSGYALHDDY | SSDVGGYNY | DVS | SSYTSSSTLYV |
| | (SEQ ID | (SEQ ID | YGLDV (SEQ | (SEQ ID | (SEQ ID | (SEQ ID |
| | NO: 90) | NO: 91) | ID NO: 92) | NO: 101) | NO: 99) | NO: 97) |
| B61-02 | GFTFSSYG | ISYKGSNK | GGSGYALHDDY | SSDVGGYNY | EVS | SSYTSSSALYV |
| | (SEQ ID | (SEQ ID | YGLDV (SEQ | (SEQ ID | (SEQ ID | (SEQ ID |
| | NO: 90) | NO: 111) | ID NO: 92) | NO: 101) | NO: 116) | NO: 115) |
| B61-10 | GFTFSSYG | ISYKGSNK | GGSGYALHDDY | SSDVGGYNY | EVS | SSYTSSSTLYV |
| | (SEQ ID | (SEQ ID | YGLDV (SEQ | (SEQ ID | (SEQ ID | (SEQ ID |
| | NO: 90) | NO: 111) | ID NO: 92) | NO: 101) | NO: 116) | NO: 97) |
| Consensus | GFTFSSYG | ISYXGSNK, | GGSGYALHDDY | SSDVGGYNY | XVS, | SSYTSSSXLYV, |
| | (SEQ ID | wherein X | YGLDV (SEQ | (SEQ ID | wherein X | wherein X is |
| | NO: 90) | is D or K | ID NO: 92) | NO: 101) | is D or E | Tor A (SEQ |
| | | (SEQ ID | | | (SEQ ID | ID NO: 132) |
| | | NO: 136) | | | NO: 134) | |

TABLE 11
Amino acid and nucleic acid sequences of exemplary anti-BCMA molecules based on PI61

| Identification | Protein sequence | DNA sequence (5′-3′) |
|---|---|---|
| Signal peptide | MALPVTALLLPLALLLHAA | Atggccctccctgtcaccgctctgttgctgccgcttgctctgctg |
| | RP (SEQ ID NO: 1) | ctccacgcagcgcgaccg (SEQ ID NO: 252) |
| ScFvPI61 | QVQLQESGGGVVQPGRSLR | CaggtacaattgcaggagtctggaggcggtgtgGtgcaacc |
| | LSCAASGFTFSSYGMHWVR | cggtcgcagcttgcgcctgagttgtGctgcgtctggatttacatt |
| | QAPGKGLEWVAVISYDGSN | ttcatcttacggaAtgcattgggtacgccaggcaccggggaa |
| | KYYADSVKGRFTISRDNSK | aggcCttgaatgggtggctgtaatttcatacgatggtTccaac |
| | NTLYLQMNSLRAEDTAVYY | aaatactatgctgactcagtcaagggtCgatttacaattagtcg |
| | CGGSGYALHDDYYGLDVW | ggacaactccaagaacAccctttatcttcaaatgaattcccttag |
| | GQGTLVTVSSGGGGSGGGG | agcaGaggatacggcggtctattactgtggtggcagtGgttat |
| | SGGGGSQSALTQPASVSGSP | gcacttcatgatgattactatggcttgGatgtctgggggcaagg |
| | GQSITISCTGTSSDVGGYNY | gacgcttgtaactgtaTcctctggtggtggtggtagtggtggg |
| | VSWYQQHPGKAPKLMIYD | ggaggcTccggcggtggcggctctcaatctgctctgactCaa |
| | VSNRPSGVSNRFSGSKSGNT | ccagcaagcgtatcagggtcaccgggacagAgtattaccata |
| | ASLTISGLQAEDEADYYCSS | agttgcacggggacctctagcGatgtaggggggtataattatg |
| | YTSSSTLYVFGSGTKVTVL | tatcttggtatCaacaacaccccgggaaagcccctaaattgatg |
| | (SEQ ID NO: 105) | AtctacgacgtgagcaatcgacctagtggcgtaTcaaatcgc |
| | | ttctctggtagcaagagtgggaatAcggcgtcccttactattag |
| | | cggattgcaagcaGaagatgaggccgattactactgcagctc |
| | | ctatActagctcttctacattgtacgtctttgggagcggaacaaa |
| | | agtaacagtactc (SEQ ID NO: 253) |
| Transmembrane | TTTPAPRPPTPAPTIASQPLS | AcaacaacacctgccccgagaccgcctacaccaGccccga |
| domain and hinge | LRPEACRPAAGGAVHTRGL | ctattgccagccagcctctgagcctcAggcctgaggcctgtag |
| | DFACDIYIWAPLAGTCGVLL | gcccgcagcgggcggcGcagttcatacacggggcttggattt |
| | LSLVITLYC | cgcttgtGatatttatatttgggctcctttggcggggacaTgtgg |
| | (SEQ ID NO: 202) | cgtgctgcttctgtcacttgttattacactgtactgt |
| | | (SEQ ID NO: 254) |
| 4-1BB | KRGRKKLLYIFKQPFMRPV | AaacgcgggcgaaaaaaattgctgtatatttttAagcagccat |
| | QTTQEEDGCSCRFPEEEEGG | ttatgaggcccgttcagacgacgCaggaggaggacggttgct |
| | CEL (SEQ ID NO: 7) | cttgcaggttcccagaagaggaagaagggggctgtgaattg |
| | | (SEQ ID NO: 255) |
| CD3zeta | RVKFSRSADAPAYQQGQNQ | CgggttaaattttcaagatccgcagacgctccaGcataccaac |
| | LYNELNLGRREEYDVLDKR | agggacaaaaccaactctataacGagctgaatcttggaagaa |
| | RGRDPEMGGKPRRKNPQEG | gggaggaatatgatGtgctggataaacggcgcggtagagatc |
| | LYNELQKDKMAEAYSEIGM | cggagAtgggcggaaaaccaaggcgaaaaaaccctcagG |
| | KGERRRGKGHDGLYQGLST | agggactctacaacgaactgcagaaagacaaaAtggcggag |
| | ATKDTYDALHMQALPPR | gcttattccgaaataggcatgaagGgcgagcggaggcgagg |
| | (SEQ ID NO: 10) | gaaagggcacgacggaCtgtatcaaggcctctcaaccgcga |
| | | ctaaggatAcgtacgacgccctgcacatgcaggccctgcctc |
| | | cgaga (SEQ ID NO: 256) |
| PI61 full CAR | MALPVTALLLPLALLLHAA | ATGGCCCTCCCTGTCACCGCTCTGTTG |
| construct | RPQVQLQESGGGVVQPGRS | CTGCCGCTTGCTCTGCTGCTCCACGCA |
| | LRLSCAASGFTFSSYGMHW | GCGCGACCGCAGGTACAATTGCAGGA |
| | VRQAPGKGLEWVAVISYDG | GTCTGGAGGCGGTGTGGTGCAACCCG |
| | SNKYYADSVKGRFTISRDNS | GTCGCAGCTTGCGCCTGAGTTGTGCTG |
| | KNTLYLQMNSLRAEDTAVY | CGTCTGGATTTACATTTTCATCTTACGG |
| | YCGGSGYALHDDYYGLDV | AATGCATTGGGTACGCCAGGCACCGG |
| | WGQGTLVTVSSGGGGSGG | GGAAAGGCCTTGAATGGGTGGCTGTA |
| | GGSGGGGSQSALTQPASVS | ATTTCATACGATGGTTCCAACAAATAC |
| | GSPGQSITISCTGTSSDVGGY | TATGCTGACTCAGTCAAGGGTCGATTT |
| | NYVSWYQQHPGKAPKLMI | ACAATTAGTCGGGACAACTCCAAGAA |
| | YDVSNRPSGVSNRFSGSKSG | CACCCTTTATCTTCAAATGAATTCCCTT |
| | NTASLTISGLQAEDEADYYC | AGAGCAGAGGATACGGCGGTCTATTA |
| | SSYTSSSTLYVFGSGTKVTV | CTGTGGTGGCAGTGGTTATGCACTTCA |
| | LTTTPAPRPPTPAPTIASQPL | TGATGATTACTATGGCTTGGATGTCTG |
| | SLRPEACRPAAGGAVHTRG | GGGGCAAGGGACGCTTGTAACTGTATC |
| | LDFACDIYIWAPLAGTCGVL | CTCTGGTGGTGGTGGTAGTGGTGGGGG |
| | LLSLVITLYCKRGRKKLLYI | AGGCTCCGGCGGTGGCGGCTCTCAATC |
| | FKQPFMRPVQTTQEEDGCS | TGCTCTGACTCAACCAGCAAGCGTATC |
| | CRFPEEEEGGCELRVKFSRS | AGGGTCACCGGGACAGAGTATTACCA |
| | ADAPAYQQGQNQLYNELN | TAAGTTGCACGGGGACCTCTAGCGATG |
| | LGRREEYDVLDKRRGRDPE | TAGGGGGGTATAATTATGTATCTTGGT |
| | MGGKPRRKNPQEGLYNELQ | ATCAACAACACCCCGGGAAAGCCCCT |
| | KDKMAEAYSEIGMKGERRR | AAATTGATGATCTACGACGTGAGCAAT |
| | GKGHDGLYQGLSTATKDTY | CGACCTAGTGGCGTATCAAATCGCTTC |
| | DALHMQALPPR (SEQ ID | TCTGGTAGCAAGAGTGGGAATACGGC |
| | NO: 257) | GTCCCTTACTATTAGCGGATTGCAAGC |
| | | AGAAGATGAGGCCGATTACTACTGCA |
| | | GCTCCTATACTAGCTCTTCTACATTGTA |
| | | CGTCTTTGGGAGCGGAACAAAAGTAA |
| | | CAGTACTCACAACAACACCTGCCCCGA |
| | | GACCGCCTACACCAGCCCCGACTATTG |
| | | CCAGCCAGCCTCTGAGCCTCAGGCCTG |
| | | AGGCCTGTAGGCCCGCAGCGGGCGGC |
| | | GCAGTTCATACACGGGGCTTGGATTTC |
| | | GCTTGTGATATTTATATTTGGGCTCCTT |
| | | TGGCGGGGACATGTGGCGTGCTGCTTC |
| | | TGTCACTTGTTATTACACTGTACTGTA |
| | | AACGCGGGCGAAAAAAATTGCTGTAT |
| | | ATTTTTAAGCAGCCATTTATGAGGCCC |
| | | GTTCAGACGACGCAGGAGGAGGACGG |
| | | TTGCTCTTGCAGGTTCCCAGAAGAGGA |
| | | AGAAGGGGGCTGTGAATTGCGGGTTA |
| | | AATTTTCAAGATCCGCAGACGCTCCAG |
| | | CATACCAACAGGGACAAAACCAACTC |
| | | TATAACGAGCTGAATCTTGGAAGAAG |
| | | GGAGGAATATGATGTGCTGGATAAAC |
| | | GGCGCGGTAGAGATCCGGAGATGGGC |
| | | GGAAAACCAAGGCGAAAAAACCCTCA |
| | | GGAGGGACTCTACAACGAACTGCAGA |
| | | AAGACAAAATGGCGGAGGCTTATTCC |
| | | GAAATAGGCATGAAGGGCGAGCGGAG |
| | | GCGAGGGAAAGGGCACGACGGACTGT |
| | | ATCAAGGCCTCTCAACCGCGACTAAGG |
| | | ATACGTACGACGCCCTGCACATGCAGG |
| | | CCCTGCCTCCGAGA (SEQ ID NO: 258) |
| PI61 mature | QVQLQESGGGVVQPGRSLR | |
| CAR protein | LSCAASGFTFSSYGMHWVR | |
| | QAPGKGLEWVAVISYDGSN | |
| | KYYADSVKGRFTISRDNSK | |
| | NTLYLQMNSLRAEDTAVYY | |
| | CGGSGYALHDDYYGLDVW | |
| | GQGTLVTVSSGGGGSGGGG | |
| | SGGGGSQSALTQPASVSGSP | |
| | GQSITISCTGTSSDVGGYNY | |
| | VSWYQQHPGKAPKLMIYD | |
| | VSNRPSGVSNRFSGSKSGNT | |
| | ASLTISGLQAEDEADYYCSS | |
| | YTSSSTLYVFGSGTKVTVLT | |
| | TTPAPRPPTPAPTIASQPLSL | |
| | RPEACRPAAGGAVHTRGLD | |
| | FACDIYIWAPLAGTCGVLLL | |
| | SLVITLYCKRGRKKLLYIFK | |
| | QPFMRPVQTTQEEDGCSCR | |
| | FPEEEEGGCELRVKFSRSAD | |
| | APAYQQGQNQLYNELNLG | |
| | RREEYDVLDKRRGRDPEMG | |
| | GKPRRKNPQEGLYNELQKD | |
| | KMAEAYSEIGMKGERRRGK | |
| | GHDGLYQGLSTATKDTYDA | |
| | LHMQALPPR | |
| | (SEQ ID NO: 107) | |

TABLE 12
Amino acid and nucleic acid sequences of exemplary
hybridoma-derived anti-BCMA molecules

| | Name/ | |
|---|---|---|
| SEQ ID NO | Description | Sequence |
| Hy03 | | |
| SEQ ID NO: 137 | HCDR1 | GFWMS |
| | (Kabat) | |
| SEQ ID NO: 138 | HCDR2 | NIKQDGSEKYYVDSVRG |
| | (Kabat) | |
| SEQ ID NO: 139 | HCDR3 | ALDYYGMDV |
| | (Kabat) | |
| SEQ ID NO: 140 | HCDR1 | GFTFSGF |
| | (Chothia) | |
| SEQ ID NO: 141 | HCDR2 | KQDGSE |
| | (Chothia) | |
| SEQ ID NO: 139 | HCDR3 | ALDYYGMDV |
| | (Chothia) | |
| SEQ ID NO: 142 | HCDR1 | GFTFSGFW |
| | (IMGT) | |
| SEQ ID NO: 143 | HCDR2 | IKQDGSEK |
| | (IMGT) | |
| SEQ ID NO: 144 | HCDR3 | ARALDYYGMDV |
| | (IMGT) | |
| SEQ ID NO: 145 | VH | EVQLVESGGGLVQPGGSLRLSCAASGFTFSGFWMSWVRQAPGKG |
| | | LEWVANIKQDGSEKYYVDSVRGRFTISRDNAKNSLYLQMNSLRAE |
| | | DTAVYYCARALDYYGMDVWGQGTTVTVSS |
| SEQ ID NO: 146 | DNA VH | GAAGTGCAACTGGTGGAGAGCGGTGGAGGGCTTGTCCAGCCCG |
| | | GAGGATCGCTGCGGCTGTCCTGTGCTGCGTCCGGGTTCACCTTC |
| | | TCCGGCTTCTGGATGTCCTGGGTCAGACAGGCACCGGGAAAGG |
| | | GCCTCGAATGGGTGGCCAACATCAAGCAGGATGGCTCCGAGAA |
| | | GTACTACGTCGACTCCGTGAGAGGCCGCTTCACCATCTCCCGGG |
| | | ACAACGCCAAGAACTCGCTGTACCTCCAAATGAATAGCCTCAG |
| | | GGCGGAAGATACTGCTGTGTATTACTGCGCACGCGCCCTTGACT |
| | | ACTACGGCATGGACGTCTGGGGCCAAGGGACCACTGTGACCGT |
| | | GTCTAGC |
| SEQ ID NO: 147 | LCDR1 | RSSQSLLDSDDGNTYLD |
| | (Kabat) | |
| SEQ ID NO: 148 | LCDR2 | TLSYRAS |
| | (Kabat) | |
| SEQ ID NO: 149 | LCDR3 | TQRLEFPSIT |
| | (Kabat) | |
| SEQ ID NO: 150 | LCDR1 | SQSLLDSDDGNTY |
| | (Chothia) | |
| SEQ ID NO: 151 | LCDR2 | TLS |
| | (Chothia) | |
| SEQ ID NO: 152 | LCDR3 | RLEFPSI |
| | (Chothia) | |
| SEQ ID NO: 153 | LCDR1 | QSLLDSDDGNTY |
| | (IMGT) | |
| SEQ ID NO: 151 | LCDR2 | TLS |
| | (IMGT) | |
| SEQ ID NO: 149 | LCDR3 | TQRLEFPSIT |
| | (IMGT) | |
| SEQ ID NO: 154 | VL | DIVMTQTPLSLPVTPGEPASISCRSSQSLLDSDDGNTYLDWYLQKP |
| | | GQSPRLLIYTLSYRASGVPDRFSGSGSGTDFTLKISRVEAEDVGLYY |
| | | CTQRLEFPSITFGQGTRLEIK |
| SEQ ID NO: 155 | DNA VL | GATATCGTGATGACCCAGACTCCCCTGTCCCTGCCTGTGACTCC |
| | | CGGAGAACCAGCCTCCATTTCCTGCCGGTCCTCCCAGTCCCTGC |
| | | TGGACAGCGACGACGGCAACACTTACCTGGACTGGTACTTGCA |
| | | GAAGCCGGGCCAATCGCCTCGCCTGCTGATCTATACCCTGTCAT |
| | | ACCGGGCCTCAGGAGTGCCTGACCGCTTCTCGGGATCAGGGAG |
| | | CGGGACCGATTTCACCCTGAAAATTTCCCGAGTGGAAGCCGAG |
| | | GACGTCGGACTGTACTACTGCACCCAGCGCCTCGAATTCCCGTC |
| | | GATTACGTTTGGACAGGGTACCCGGCTTGAGATCAAG |
| SEQ ID NO: 63 | Linker | GGGGSGGGGSGGGGSGGGGS |
| SEQ ID NO: 156 | scFv (VH- | EVQLVESGGGLVQPGGSLRLSCAASGFTFSGFWMSWVRQAPGKG |
| | linker-VL) | LEWVANIKQDGSEKYYVDSVRGRFTISRDNAKNSLYLQMNSLRAE |
| | | DTAVYYCARALDYYGMDVWGQGTTVTVSSGGGGSGGGGSGGG |
| | | GSGGGGSDIVMTQTPLSLPVTPGEPASISCRSSQSLLDSDDGNTYLD |
| | | WYLQKPGQSPRLLIYTLSYRASGVPDRFSGSGSGTDFTLKISRVEA |
| | | EDVGLYYCTQRLEFPSITFGQGTRLEIK |
| SEQ ID NO: 157 | DNA scFv | GAAGTGCAACTGGTGGAGAGCGGTGGAGGGCTTGTCCAGCCCG |
| | | GAGGATCGCTGCGGCTGTCCTGTGCTGCGTCCGGGTTCACCTTC |
| | | TCCGGCTTCTGGATGTCCTGGGTCAGACAGGCACCGGGAAAGG |
| | | GCCTCGAATGGGTGGCCAACATCAAGCAGGATGGCTCCGAGAA |
| | | GTACTACGTCGACTCCGTGAGAGGCCGCTTCACCATCTCCCGGG |
| | | ACAACGCCAAGAACTCGCTGTACCTCCAAATGAATAGCCTCAG |
| | | GGCGGAAGATACTGCTGTGTATTACTGCGCACGCGCCCTTGACT |
| | | ACTACGGCATGGACGTCTGGGGCCAAGGGACCACTGTGACCGT |
| | | GTCTAGCGGAGGCGGAGGTTCAGGGGGCGGTGGATCAGGCGGA |
| | | GGAGGATCGGGGGGTGGTGGATCGGATATCGTGATGACCCAGA |
| | | CTCCCCTGTCCCTGCCTGTGACTCCCGGAGAACCAGCCTCCATT |
| | | TCCTGCCGGTCCTCCCAGTCCCTGCTGGACAGCGACGACGGCAA |
| | | CACTTACCTGGACTGGTACTTGCAGAAGCCGGGCCAATCGCCTC |
| | | GCCTGCTGATCTATACCCTGTCATACCGGGCCTCAGGAGTGCCT |
| | | GACCGCTTCTCGGGATCAGGGAGCGGGACCGATTTCACCCTGA |
| | | AAATTTCCCGAGTGGAAGCCGAGGACGTCGGACTGTACTACTG |
| | | CACCCAGCGCCTCGAATTCCCGTCGATTACGTTTGGACAGGGTA |
| | | CCCGGCTTGAGATCAAG |
| SEQ ID NO: 158 | Full CAR | EVQLVESGGGLVQPGGSLRLSCAASGFTFSGFWMSWVRQAPGKG |
| | amino acid | LEWVANIKQDGSEKYYVDSVRGRFTISRDNAKNSLYLQMNSLRAE |
| | sequence | DTAVYYCARALDYYGMDVWGQGTTVTVSSGGGGSGGGGSGGG |
| | | GSGGGGSDIVMTQTPLSLPVTPGEPASISCRSSQSLLDSDDGNTYLD |
| | | WYLQKPGQSPRLLIYTLSYRASGVPDRFSGSGSGTDFTLKISRVEA |
| | | EDVGLYYCTQRLEFPSITFGQGTRLEIKTTFPAPRPPTPAPTIASQPL |
| | | SLRPEACRPAAGGAVHTRGLDFACDIYIWAPLAGTCGVLLLSLVIT |
| | | LYCKRGRKKLLYIFKQPFMRPVQTTQEEDGCSCRFPEEEEGGCELR |
| | | VKFSRSADAPAYQQGQNQLYNELNLGRREEYDVLDKRRGRDPEM |
| | | GGKPRRKNPQEGLYNELQKDKMAEAYSEIGMKGERRRGKGHDG |
| | | LYQGLSTATKDTYDALHMQALPPR |
| SEQ ID NO: 159 | Full CAR | GAAGTGCAACTGGTGGAGAGCGGTGGAGGGCTTGTCCAGCCCG |
| | DNA | GAGGATCGCTGCGGCTGTCCTGTGCTGCGTCCGGGTTCACCTTC |
| | sequence | TCCGGCTTCTGGATGTCCTGGGTCAGACAGGCACCGGGAAAGG |
| | | GCCTCGAATGGGTGGCCAACATCAAGCAGGATGGCTCCGAGAA |
| | | GTACTACGTCGACTCCGTGAGAGGCCGCTTCACCATCTCCCGGG |
| | | ACAACGCCAAGAACTCGCTGTACCTCCAAATGAATAGCCTCAG |
| | | GGCGGAAGATACTGCTGTGTATTACTGCGCACGCGCCCTTGACT |
| | | ACTACGGCATGGACGTCTGGGGCCAAGGGACCACTGTGACCGT |
| | | GTCTAGCGGAGGCGGAGGTTCAGGGGGCGGTGGATCAGGCGGA |
| | | GGAGGATCGGGGGGTGGTGGATCGGATATCGTGATGACCCAGA |
| | | CTCCCCTGTCCCTGCCTGTGACTCCCGGAGAACCAGCCTCCATT |
| | | TCCTGCCGGTCCTCCCAGTCCCTGCTGGACAGCGACGACGGCAA |
| | | CACTTACCTGGACTGGTACTTGCAGAAGCCGGGCCAATCGCCTC |
| | | GCCTGCTGATCTATACCCTGTCATACCGGGCCTCAGGAGTGCCT |
| | | GACCGCTTCTCGGGATCAGGGAGCGGGACCGATTTCACCCTGA |
| | | AAATTTCCCGAGTGGAAGCCGAGGACGTCGGACTGTACTACTG |
| | | CACCCAGCGCCTCGAATTCCCGTCGATTACGTTTGGACAGGGTA |
| | | CCCGGCTTGAGATCAAGACCACTACCCCAGCACCGAGGCCACC |
| | | CACCCCGGCTCCTACCATCGCCTCCCAGCCTCTGTCCCTGCGTC |
| | | CGGAGGCATGTAGACCCGCAGCTGGTGGGGCCGTGCATACCCG |
| | | GGGTCTTGACTTCGCCTGCGATATCTACATTTGGGCCCCTCTGG |
| | | CTGGTACTTGCGGGGTCCTGCTGCTTTCACTCGTGATCACTCTTT |
| | | ACTGTAAGCGCGGTCGGAAGAAGCTGCTGTACATCTTTAAGCA |
| | | ACCCTTCATGAGGCCTGTGCAGACTACTCAAGAGGAGGACGGC |
| | | TGTTCATGCCGGTTCCCAGAGGAGGAGGAAGGCGGCTGCGAAC |
| | | TGCGCGTGAAATTCAGCCGCAGCGCAGATGCTCCAGCCTACCA |
| | | GCAGGGGCAGAACCAGCTCTACAACGAACTCAATCTTGGTCGG |
| | | AGAGAGGAGTACGACGTGCTGGACAAGCGGAGAGGACGGGAC |
| | | CCAGAAATGGGCGGGAAGCCGCGCAGAAAGAATCCCCAAGAG |
| | | GGCCTGTACAACGAGCTCCAAAAGGATAAGATGGCAGAAGCCT |
| | | ATAGCGAGATTGGTATGAAAGGGGAACGCAGAAGAGGCAAAG |
| | | GCCACGACGGACTGTACCAGGGACTCAGCACCGCCACCAAGGA |
| | | CACCTATGACGCTCTTCACATGCAGGCCCTGCCGCCTCGG |
| Hy52 | | |
| SEQ ID NO: 160 | HCDR1 | SFRMN |
| | (Kabat) | |
| SEQ ID NO: 161 | HCDR2 | SISSSSSYIYYADSVKG |
| | (Kabat) | |
| SEQ ID NO: 162 | HCDR3 | WLSYYGMDV |
| | (Kabat) | |
| SEQ ID NO: 163 | HCDR1 | GFTFSSF |
| | (Chothia) | |
| SEQ ID NO: 164 | HCDR2 | SSSSSY |
| | (Chothia) | |
| SEQ ID NO: 162 | HCDR3 | WLSYYGMDV |
| | (Chothia) | |
| SEQ ID NO: 165 | HCDR1 | GFTFSSFR |
| | (IMGT) | |
| SEQ ID NO: 166 | HCDR2 | ISSSSSYI |
| | (IMGT) | |
| SEQ ID NO: 167 | HCDR3 | ARWLSYYGMDV |
| | (IMGT) | |
| SEQ ID NO: 168 | VH | EVQLVESGGGLVKPGGSLRLSCAASGFTFSSFRMNWVRQAPGKGL |
| | | EWVSSISSSSSYIYYADSVKGRFTISRDNAKNSLYLQMNSLRAEDT |
| | | AVYYCARWLSYYGMDVWGQGTTVTVSS |
| SEQ ID NO: 169 | DNA VH | GAAGTGCAACTGGTGGAGAGCGGTGGAGGGCTTGTCAAGCCCG |
| | | GAGGATCGCTGCGGCTGTCCTGTGCTGCGTCCGGGTTCACCTTC |
| | | TCCTCGTTCCGCATGAACTGGGTCAGACAGGCACCGGGAAAGG |
| | | GCCTCGAATGGGTGTCCTCAATCTCATCGTCCTCGTCCTACATC |
| | | TACTACGCCGACTCCGTGAAAGGCCGCTTCACCATCTCCCGGGA |
| | | CAACGCCAAGAACTCGCTGTACCTCCAAATGAATAGCCTCAGG |
| | | GCGGAAGATACTGCTGTGTATTACTGCGCACGCTGGCTTTCCTA |
| | | CTACGGCATGGACGTCTGGGGCCAAGGGACCACTGTGACCGTG |
| | | TCTAGC |
| SEQ ID NO: 147 | LCDR1 | RSSQSLLDSDDGNTYLD |
| | (Kabat) | |
| SEQ ID NO: 170 | LCDR2 | TLSFRAS |
| | (Kabat) | |
| SEQ ID NO: 171 | LCDR3 | MQRIGFPIT |
| | (Kabat) | |
| SEQ ID NO: 150 | LCDR1 | SQSLLDSDDGNTY |
| | (Chothia) | |
| SEQ ID NO: 151 | LCDR2 | TLS |
| | (Chothia) | |
| SEQ ID NO: 172 | LCDR3 | RIGFPI |
| | (Chothia) | |
| SEQ ID NO: 153 | LCDR1 | QSLLDSDDGNTY |
| | (IMGT) | |
| SEQ ID NO: 151 | LCDR2 | TLS |
| | (IMGT) | |
| SEQ ID NO: 171 | LCDR3 | MQRIGFPIT |
| | (IMGT) | |
| SEQ ID NO: 173 | VL | DIVMTQTPLSLPVTPGEPASISCRSSQSLLDSDDGNTYLDWYLQKP |
| | | GQSPQLLIYTLSFRASGVPDRFSGSGSGTDFTLKIRRVEAEDVGVY |
| | | YCMQRIGFPITFGQGTRLEIK |
| SEQ ID NO: 174 | DNA VL | GATATCGTGATGACCCAGACTCCCCTGTCCCTGCCTGTGACTCC |
| | | CGGAGAACCAGCCTCCATTTCCTGCCGGTCCTCCCAGTCCCTGC |
| | | TGGACAGCGACGACGGCAACACTTACCTGGACTGGTACTTGCA |
| | | GAAGCCGGGCCAATCGCCTCAGCTGCTGATCTATACCCTGTCAT |
| | | TCCGGGCCTCAGGAGTGCCTGACCGCTTCTCGGGATCAGGGAG |
| | | CGGGACCGATTTCACCCTGAAAATTAGGCGAGTGGAAGCCGAG |
| | | GACGTCGGAGTGTACTACTGCATGCAGCGCATCGGCTTCCCGAT |
| | | TACGTTTGGACAGGGTACCCGGCTTGAGATCAAG |
| SEQ ID NO: 63 | Linker | GGGGSGGGGSGGGGSGGGGS |
| SEQ ID NO: 175 | scFv (VH- | EVQLVESGGGLVKPGGSLRLSCAASGFTFSSFRMNWVRQAPGKGL |
| | linker-VL) | EWVSSISSSSSYIYYADSVKGRFTISRDNAKNSLYLQMNSLRAEDT |
| | | AVYYCARWLSYYGMDVWGQGTTVTVSSGGGGSGGGGSGGGGS |
| | | GGGGSDIVMTQTPLSLPVTPGEPASISCRSSQSLLDSDDGNTYLDW |
| | | YLQKPGQSPQLLIYTLSFRASGVPDRFSGSGSGTDFTLKIRRVEAED |
| | | VGVYYCMQRIGFPITFGQGTRLEIK |
| SEQ ID NO: 176 | DNA scFv | GAAGTGCAACTGGTGGAGAGCGGTGGAGGGCTTGTCAAGCCCG |
| | | GAGGATCGCTGCGGCTGTCCTGTGCTGCGTCCGGGTTCACCTTC |
| | | TCCTCGTTCCGCATGAACTGGGTCAGACAGGCACCGGGAAAGG |
| | | GCCTCGAATGGGTGTCCTCAATCTCATCGTCCTCGTCCTACATC |
| | | TACTACGCCGACTCCGTGAAAGGCCGCTTCACCATCTCCCGGGA |
| | | CAACGCCAAGAACTCGCTGTACCTCCAAATGAATAGCCTCAGG |
| | | GCGGAAGATACTGCTGTGTATTACTGCGCACGCTGGCTTTCCTA |
| | | CTACGGCATGGACGTCTGGGGCCAAGGGACCACTGTGACCGTG |
| | | TCTAGCGGAGGCGGAGGTTCAGGGGGCGGTGGATCAGGCGGAG |
| | | GAGGATCGGGGGGTGGTGGATCGGATATCGTGATGACCCAGAC |
| | | TCCCCTGTCCCTGCCTGTGACTCCCGGAGAACCAGCCTCCATTT |
| | | CCTGCCGGTCCTCCCAGTCCCTGCTGGACAGCGACGACGGCAA |
| | | CACTTACCTGGACTGGTACTTGCAGAAGCCGGGCCAATCGCCTC |
| | | AGCTGCTGATCTATACCCTGTCATTCCGGGCCTCAGGAGTGCCT |
| | | GACCGCTTCTCGGGATCAGGGAGCGGGACCGATTTCACCCTGA |
| | | AAATTAGGCGAGTGGAAGCCGAGGACGTCGGAGTGTACTACTG |
| | | CATGCAGCGCATCGGCTTCCCGATTACGTTTGGACAGGGTACCC |
| | | GGCTTGAGATCAAG |
| SEQ ID NO: 177 | Full CAR | EVQLVESGGGLVKPGGSLRLSCAASGFTFSSFRMNWVRQAPGKGL |
| | amino acid | EWVSSISSSSSYIYYADSVKGRFTISRDNAKNSLYLQMNSLRAEDT |
| | sequence | AVYYCARWLSYYGMDVWGQGTTVTVSSGGGGSGGGGSGGGGS |
| | | GGGGSDIVMTQTPLSLPVTPGEPASISCRSSQSLLDSDDGNTYLDW |
| | | YLQKPGQSPQLLIYTLSFRASGVPDRFSGSGSGTDFTLKIRRVEAED |
| | | VGVYYCMQRIGFPITFGQGTRLEIKTTTPAPRPPTPAPTIASQPLSLR |
| | | PEACRPAAGGAVHTRGLDFACDIYIWAPLAGTCGVLLLSLVITLYC |
| | | KRGRKKLLYIFKQPFMRPVQTTQEEDGCSCRFPEEEEGGCELRVKF |
| | | SRSADAPAYQQGQNQLYNELNLGRREEYDVLDKRRGRDPEMGG |
| | | KPRRKNPQEGLYNELQKDKMAEAYSEIGMKGERRRGKGHDGLY |
| | | QGLSTATKDTYDALHMQALPPR |
| SEQ ID NO: 178 | Full CAR | GAAGTGCAACTGGTGGAGAGCGGTGGAGGGCTTGTCAAGCCCG |
| | DNA | GAGGATCGCTGCGGCTGTCCTGTGCTGCGTCCGGGTTCACCTTC |
| | sequence | TCCTCGTTCCGCATGAACTGGGTCAGACAGGCACCGGGAAAGG |
| | | GCCTCGAATGGGTGTCCTCAATCTCATCGTCCTCGTCCTACATC |
| | | TACTACGCCGACTCCGTGAAAGGCCGCTTCACCATCTCCCGGGA |
| | | CAACGCCAAGAACTCGCTGTACCTCCAAATGAATAGCCTCAGG |
| | | GCGGAAGATACTGCTGTGTATTACTGCGCACGCTGGCTTTCCTA |
| | | CTACGGCATGGACGTCTGGGGCCAAGGGACCACTGTGACCGTG |
| | | TCTAGCGGAGGCGGAGGTTCAGGGGGCGGTGGATCAGGCGGAG |
| | | GAGGATCGGGGGGTGGTGGATCGGATATCGTGATGACCCAGAC |
| | | TCCCCTGTCCCTGCCTGTGACTCCCGGAGAACCAGCCTCCATTT |
| | | CCTGCCGGTCCTCCCAGTCCCTGCTGGACAGCGACGACGGCAA |
| | | CACTTACCTGGACTGGTACTTGCAGAAGCCGGGCCAATCGCCTC |
| | | AGCTGCTGATCTATACCCTGTCATTCCGGGCCTCAGGAGTGCCT |
| | | GACCGCTTCTCGGGATCAGGGAGCGGGACCGATTTCACCCTGA |
| | | AAATTAGGCGAGTGGAAGCCGAGGACGTCGGAGTGTACTACTG |
| | | CATGCAGCGCATCGGCTTCCCGATTACGTTTGGACAGGGTACCC |
| | | GGCTTGAGATCAAGACCACTACCCCAGCACCGAGGCCACCCAC |
| | | CCCGGCTCCTACCATCGCCTCCCAGCCTCTGTCCCTGCGTCCGG |
| | | AGGCATGTAGACCCGCAGCTGGTGGGGCCGTGCATACCCGGGG |
| | | TCTTGACTTCGCCTGCGATATCTACATTTGGGCCCCTCTGGCTG |
| | | GTACTTGCGGGGTCCTGCTGCTTTCACTCGTGATCACTCTTTACT |
| | | GTAAGCGCGGTCGGAAGAAGCTGCTGTACATCTTTAAGCAACC |
| | | CTTCATGAGGCCTGTGCAGACTACTCAAGAGGAGGACGGCTGT |
| | | TCATGCCGGTTCCCAGAGGAGGAGGAAGGCGGCTGCGAACTGC |
| | | GCGTGAAATTCAGCCGCAGCGCAGATGCTCCAGCCTACCAGCA |
| | | GGGGCAGAACCAGCTCTACAACGAACTCAATCTTGGTCGGAGA |
| | | GAGGAGTACGACGTGCTGGACAAGCGGAGAGGACGGGACCCA |
| | | GAAATGGGCGGGAAGCCGCGCAGAAAGAATCCCCAAGAGGGC |
| | | CTGTACAACGAGCTCCAAAAGGATAAGATGGCAGAAGCCTATA |
| | | GCGAGATTGGTATGAAAGGGGAACGCAGAAGAGGCAAAGGCC |
| | | ACGACGGACTGTACCAGGGACTCAGCACCGCCACCAAGGACAC |
| | | CTATGACGCTCTTCACATGCAGGCCCTGCCGCCTCGG |

TABLE 13
Kabat CDRs of exemplary hybridoma-derived anti-BCMA molecules

| Kabat | HCDR1 | HCDR2 | HCDR3 | LCDR1 | LCDR2 | LCDR3 |
|---|---|---|---|---|---|---|
| Hy03 | GFWMS | NIKQDGSEKYYVDSVR | ALDYYGMDV | RSSQSLLDS | TLSYRAS | TQRLEFPSIT |
| | (SEQ ID | G (SEQ ID | (SEQ ID | DDGNTYLD | (SEQ ID | (SEQ ID |
| | NO: 137) | NO:138) | NO: 139) | (SEQ ID | NO: 148) | NO: 149) |
| | | | | NO: 147) | | |
| Hy52 | SFRMN | SISSSSSYIYYADSVK | WLSYYGMDV | RSSQSLLDS | TLSFRAS | MQRIGFPIT |
| | (SEQ ID | G (SEQ ID | (SEQ ID | DDGNTYLD | (SEQ ID | (SEQ ID |
| | NO: 160) | NO: 161) | NO: 162) | (SEQ ID | NO: 170) | NO: 171) |
| | | | | NO: 147) | | |
| Consensus | X₁FX₂MX₃, | X₁IX₂X₃X₄X₅SX₆X₇YY | X₁LX₂YYGMDV, | RSSQSLLDS | TLSXRAS, | X₁QRX₂X₃FPX₄ |
| | wherein | X₈DSVXG, wherein | wherein | DDGNTYLD | wherein | IT, |
| | X₁ is G or S; | X₁ is N or S; | X₁ is A or W; | (SEQ ID | X is Y or | wherein |
| | X₂ is W or 4; | X₂ is K or S; | and X₂ is D or | NO: 147) | F (SEQ ID | X₁ is T or M; |
| | and | X₃ is Q or S; | S (SEQ ID | | NO: 182) | X₂ is L or I; |
| | X₃ is S or N | X₄ is D or S; | NO: 181) | | | X₃ is E or G; |
| | (SEQ ID | X₅ is G or S; | | | | and X₄ is S |
| | NO: 179) | X₆ is E or Y; | | | | or absent |
| | | X₇ is K or I; | | | | (SEQ ID |
| | | X₈ is V or A; and | | | | NO: 183) |
| | | X₉ is R or K | | | | |
| | | (SEQ ID NO: 180) | | | | |

TABLE 14
Chothia CDRs of exemplary hybridoma-derived anti-BCMA molecules

| Chothia | HCDR1 | HCDR2 | HCDR3 | LCDR1 | LCDR2 | LCDR3 |
|---|---|---|---|---|---|---|
| Hy03 | GFTFSGF | KQDGSE | ALDYYGMDV | SQSLLDSD | TLS | RLEFPSI |
| | (SEQ ID | (SEQ ID NO: 141) | (SEQ ID | DGNTY | (SEQ ID | (SEQ ID |
| | NO: 140) | | NO: 139) | (SEQ ID | NO: 151) | NO: 152) |
| | | | | NO: 150) | | |
| Hy52 | GFTFSSF | SSSSSY | WLSYYGMDV | SQSLLDSD | TLS | RIGFPI |
| | (SEQ ID | (SEQ ID NO: 164) | (SEQ ID | DGNTY | (SEQ ID | (SEQ ID |
| | NO: 163) | | NO: 162) | (SEQ ID | NO: 151) | NO: 172) |
| | | | | NO: 150) | | |
| Consensus | GFTFSXF, | X₁X₂X₃X₄SX₅, | X₁LX₂YYGMDV, | SQSLLDSD | TLS | RX₁X₂FPX₃I, |
| | wherein X is | wherein X₁ is K | wherein | DGNTY | (SEQ ID | wherein |
| | G or S (SEQ | or S; X₂ is Q or | X₁ is A or W; | (SEQ ID | NO: 151) | X₁ is L or |
| | ID NO: 184) | S; X₃ is D or S; | and X₂ is D or | NO: 150) | | I; X₂ is E |
| | | X₄ is G or S; and | S (SEQ ID | | | or G; and |
| | | X₅ is E or Y | NO: 181) | | | X₃ is S or |
| | | (SEQ ID NO: 185) | | | | absent |
| | | | | | | (SEQ ID |
| | | | | | | NO: 186) |

TABLE 15
IMGT CDRs of exemplary hybridoma-derived anti-BCMA molecules

| IMGT | HCDR1 | HCDR2 | HCDR3 | LCDR1 | LCDR2 | LCDR3 |
|---|---|---|---|---|---|---|
| Hy03 | GFTFSGFW | IKQDGSEK | ARALDYYG | QSLLDSDDGNTY | TLS | TQRLEFPSIT |
| | (SEQ ID | (SEQ ID NO: | MDV (SEQ | (SEQ ID | (SEQ ID | (SEQ ID |
| | NO: 142) | 143) | ID NO: 144) | NO: 153) | NO: 151) | NO: 149) |
| Hy52 | GFTFSSFR | ISSSSSYI | ARWLSYYG | QSLLDSDDGNTY | TLS | MQRIGFPIT |
| | (SEQ ID | (SEQ ID NO: | MDV (SEQ | (SEQ ID | (SEQ ID | (SEQ ID |
| | NO: 165) | 166) | ID NO: 167) | NO: 153) | NO: 151) | NO: 171) |
| Consensus | GFTFSX₁F | IX₁X₂X₃X₄SX₅ | ARX₁LX₂YY | QSLLDSDDGNTY | TLS | X₁QRX₂X₃FPX₄IT, |
| | X₂, wherein | X₆, wherein | GMDV, | (SEQ ID | (SEQ ID | wherein X₁ |
| | X₁ is G or S; | X₁ is K or S; | wherein X₁ is | NO: 153) | NO: 151) | is T or M; |
| | and X₂ is W | X₂ is Q or S; | A or W; and | | | X2 is L or I; |
| | or R (SEQ | X₃ is D or S; | X₂ is D or S | | | X3 is E or |
| | ID NO: 187) | X₄ is G or S; | (SEQ ID | | | G; and X4 is |
| | | X₅ is E or Y; | NO: 189) | | | S or absent |
| | | and X₆ is K or | | | | (SEQ ID |
| | | I (SEQ ID | | | | NO: 183) |
| | | NO: 188) | | | | |

In some embodiments, the human anti-BCMA binding domain comprises a HC CDR1, HC CDR2, HC CDR3, LC CDR1, LC CDR2, and LC CDR3.

In certain embodiments, the CAR molecule described herein or the anti-BCMA binding domain described herein includes:

(1) one, two, or three light chain (LC) CDRs chosen from:

(i) a LC CDR1 of SEQ ID NO: 54, LC CDR2 of SEQ ID NO: 55 and LC CDR3 of SEQ ID NO: 56; and/or

(2) one, two, or three heavy chain (HC) CDRs from one of the following:

(i) a HC CDR1 of SEQ ID NO: 44, HC CDR2 of SEQ ID NO: 45 and HC CDR3 of SEQ ID NO: 84; (ii) a HC CDR1 of SEQ ID NO: 44, HC CDR2 of SEQ ID NO: 45 and HC CDR3 of SEQ ID NO: 46; (iii) a HC CDR1 of SEQ ID NO: 44, HC CDR2 of SEQ ID NO: 45 and HC CDR3 of SEQ ID NO: 68; or (iv) a HC CDR1 of SEQ ID NO: 44, HC CDR2 of SEQ ID NO: 45 and HC CDR3 of SEQ ID NO: 76.

In certain embodiments, the CAR molecule described herein or the anti-BCMA binding domain described herein includes:

(1) one, two, or three light chain (LC) CDRs from one of the following:

(i) a LC CDR1 of SEQ ID NO: 95, LC CDR2 of SEQ ID NO: 131 and LC CDR3 of SEQ ID NO: 132; (ii) a LC CDR1 of SEQ ID NO: 95, LC CDR2 of SEQ ID NO: 96 and LC CDR3 of SEQ ID NO: 97; (iii) a LC CDR1 of SEQ ID NO: 95, LC CDR2 of SEQ ID NO: 114 and LC CDR3 of SEQ ID NO: 115; or (iv) a LC CDR1 of SEQ ID NO: 95, LC CDR2 of SEQ ID NO: 114 and LC CDR3 of SEQ ID NO: 97; and/or

(2) one, two, or three heavy chain (HC) CDRs from one of the following:

(i) a HC CDR1 of SEQ ID NO: 86, HC CDR2 of SEQ ID NO: 130 and HC CDR3 of SEQ ID NO: 88; (ii) a HC CDR1 of SEQ ID NO: 86, HC CDR2 of SEQ ID NO: 87 and HC CDR3 of SEQ ID NO: 88; or (iii) a HC CDR1 of SEQ ID NO: 86, HC CDR2 of SEQ ID NO: 109 and HC CDR3 of SEQ ID NO: 88.

In certain embodiments, the CAR molecule described herein or the anti-BCMA binding domain described herein includes:

(1) one, two, or three light chain (LC) CDRs from one of the following:

(i) a LC CDR1 of SEQ ID NO: 147, LC CDR2 of SEQ ID NO: 182 and LC CDR3 of SEQ ID NO: 183; (ii) a LC CDR1 of SEQ ID NO: 147, LC CDR2 of SEQ ID NO: 148 and LC CDR3 of SEQ ID NO: 149; or (iii) a LC CDR1 of SEQ ID NO: 147, LC CDR2 of SEQ ID NO: 170 and LC CDR3 of SEQ ID NO: 171; and/or

(2) one, two, or three heavy chain (HC) CDRs from one of the following:

(i) a HC CDR1 of SEQ ID NO: 179, HC CDR2 of SEQ ID NO: 180 and HC CDR3 of SEQ ID NO: 181; (ii) a HC CDR1 of SEQ ID NO: 137, HC CDR2 of SEQ ID NO: 138 and HC CDR3 of SEQ ID NO: 139; or (iii) a HC CDR1 of SEQ ID NO: 160, HC CDR2 of SEQ ID NO: 161 and HC CDR3 of SEQ ID NO: 162.

In some embodiments, the HC CDR1, HC CDR2, HC CDR3, LC CDR1, LC CDR2, and LC CDR3 comprise the amino acid sequences of SEQ ID NOs: 44, 45, 84, 54, 55, and 56, respectively. In some embodiments, the HC CDR1, HC CDR2, HC CDR3, LC CDR1, LC CDR2, and LC CDR3 comprise the amino acid sequences of SEQ ID NOs: 44, 45, 46, 54, 55, and 56, respectively. In some embodiments, the HC CDR1, HC CDR2, HC CDR3, LC CDR1, LC CDR2, and LC CDR3 comprise the amino acid sequences of SEQ ID NOs: 44, 45, 68, 54, 55, and 56, respectively. In some embodiments, the HC CDR1, HC CDR2, HC CDR3, LC CDR1, LC CDR2, and LC CDR3 comprise the amino acid sequences of SEQ ID NOs: 44, 45, 76, 54, 55, and 56, respectively.

In some embodiments, the HC CDR1, HC CDR2, HC CDR3, LC CDR1, LC CDR2, and LC CDR3 comprise the amino acid sequences of SEQ ID NOs: 47, 48, 84, 57, 58, and 59, respectively. In some embodiments, the HC CDR1, HC CDR2, HC CDR3, LC CDR1, LC CDR2, and LC CDR3 comprise the amino acid sequences of SEQ ID NOs: 47, 48, 46, 57, 58, and 59, respectively. In some embodiments, the HC CDR1, HC CDR2, HC CDR3, LC CDR1, LC CDR2, and LC CDR3 comprise the amino acid sequences of SEQ ID NOs: 47, 48, 68, 57, 58, and 59, respectively. In some embodiments, the HC CDR1, HC CDR2, HC CDR3, LC CDR1, LC CDR2, and LC CDR3 comprise the amino acid sequences of SEQ ID NOs: 47, 48, 76, 57, 58, and 59, respectively.

In some embodiments, the HC CDR1, HC CDR2, HC CDR3, LC CDR1, LC CDR2, and LC CDR3 comprise the amino acid sequences of SEQ ID NOs: 49, 50, 85, 60, 58, and 56, respectively. In some embodiments, the HC CDR1, HC CDR2, HC CDR3, LC CDR1, LC CDR2, and LC CDR3 comprise the amino acid sequences of SEQ ID NOs: 49, 50, 51, 60, 58, and 56, respectively. In some embodiments, the HC CDR1, HC CDR2, HC CDR3, LC CDR1, LC CDR2, and LC CDR3 comprise the amino acid sequences of SEQ ID NOs: 49, 50, 69, 60, 58, and 56, respectively. In some embodiments, the HC CDR1, HC CDR2, HC CDR3, LC CDR1, LC CDR2, and LC CDR3 comprise the amino acid sequences of SEQ ID NOs: 49, 50, 77, 60, 58, and 56, respectively.

In some embodiments, the human anti-BCMA binding domain comprises a scFv comprising a VH (for example, a VH described herein) and VL (for example, a VL described herein). In some embodiments, the VH is attached to the VL via a linker, for example, a linker described herein, for example, a linker described in Table 1. In some embodiments, the human anti-BCMA binding domain comprises a (Gly₄-Ser)n linker, wherein n is 1, 2, 3, 4, 5, or 6, preferably 3 or 4 (SEQ ID NO: 26). The light chain variable region and heavy chain variable region of a scFv can be, for example, in any of the following orientations: light chain variable region-linker-heavy chain variable region or heavy chain variable region-linker-light chain variable region.

In some embodiments, the anti-BCMA binding domain is a fragment, for example, a single chain variable fragment (scFv). In some embodiments, the anti-BCMA binding domain is a Fv, a Fab, a (Fab)2, or a bi-functional (for example bi-specific) hybrid antibody (for example, Lanzavecchia et al., Eur. J. Immunol. 17, 105 (1987)). In some embodiments, the antibodies and fragments thereof of this disclosure binds a BCMA protein with wild-type or enhanced affinity.

In some instances, scFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (for example, a Ser-Gly linker) with an optimized length and/or amino acid composition. The linker length can greatly affect how the variable regions of a scFv fold and interact. In fact, if a short polypeptide linker is employed (for example, between 5-10 amino acids) intrachain folding is prevented. Interchain folding is also required to bring the two variable regions together to form a functional epitope binding site. For examples of linker orientation and size see, for example, Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In some embodiments, the linker sequence comprises sets of glycine and serine repeats such as (Gly₄Ser)n, where n is a positive integer equal to or greater than 1 (SEQ ID NO: 25). In some embodiments, the linker can be (Gly₄Ser)₄ (SEQ ID NO: 27) or (Gly₄Ser)₃(SEQ ID NO: 28). Variation in the linker length may retain or enhance activity, giving rise to superior efficacy in activity studies.

### CD20 CAR

In some embodiments, the CAR-expressing cell described herein is a CD20 CAR-expressing cell (for example, a cell expressing a CAR that binds to human CD20). In some embodiments, the CD20 CAR-expressing cell includes an antigen binding domain according to WO2016164731 and WO2018067992, incorporated herein by reference. Exemplary CD20-binding sequences or CD20 CAR sequences are disclosed in, for example, Tables 1-5 of WO2018067992. In some embodiments, the CD20 CAR comprises a CDR, variable region, scFv, or full-length sequence of a CD20 CAR disclosed in WO2018067992 or WO2016164731.

### CD22 CAR

In some embodiments, the CAR-expressing cell described herein is a CD22 CAR-expressing cell (for example, a cell expressing a CAR that binds to human CD22). In some embodiments, the CD22 CAR-expressing cell includes an antigen binding domain according to WO2016164731 and WO2018067992, incorporated herein by reference. Exemplary CD22-binding sequences or CD22 CAR sequences are disclosed in, for example, Tables 6A, 6B, 7A, 7B, 7C, 8A, 8B, 9A, 9B, 10A, and 10B of WO2016164731 and Tables 6-10 of WO2018067992. In some embodiments, the CD22 CAR sequences comprise a CDR, variable region, scFv or full-length sequence of a CD22 CAR disclosed in WO2018067992 or WO2016164731.

In embodiments, the CAR molecule comprises an antigen binding domain that binds to CD22 (CD22 CAR). In some embodiments, the antigen binding domain targets human CD22. In some embodiments, the antigen binding domain includes a single chain Fv sequence as described herein.

The sequences of human CD22 CAR are provided below. In some embodiments, a human CD22 CAR is CAR22-65.

Human CD22 CAR scFv sequence
(SEQ ID NO: 285)
EVQLQQSGPGLVKPSQTLSLTCAISGDSMLSNSDTWNWIRQSPSRGLEW
LGRTYHRSTWYDDYASSVRGRVSINVDTSKNQYSLQLNAVTPEDTGVYY
CARVRLQDGNSWSDAFDVWGQGTMVTVSSGGGGSGGGGSGGGGSQSALT
QPASASGSPGQSVTISCTGTSSDVGGYNYVSWYQQHPGKAPKLMIYDVS
NRPSGVSNRFSGSKSGNTASLTISGLQAEDEADYYCSSYTSSSTLYVFG
TGTQLTVL
Human CD22 CAR heavy chain variable region
(SEQ ID NO 286)
EVQLQQSGPGLVKPSQTLSLTCAISGDSMLSNSDTWNWIRQSPSRGLEW
LGRTYHRSTWYDDYASSVRGRVSINVDTSKNQYSLQLNAVTPEDTGVYY
CARVRLQDGNSWSDAFDVWGQGTMVTVSS
Human CD22 CAR light chain variable region
(SEQ ID NO 287)
QSALTQPASASGSPGQSVTISCTGTSSDVGGYNYVSWYQQHPGKAPKLM
IYDVSNRPSGVSNRFSGSKSGNTASLTISGLQAEDEADYYCSSYTSSST
LYVFGTGTQLTVL

TABLE 16
Heavy Chain Variable Domain CDRs
of CD22 CAR (CAR22-65)

| | | SEQ | | SEQ | | SEQ |
|---|---|---|---|---|---|---|
| Candidate | HCDR1 | ID NO: | HCDR2 | ID NO: | HCDR3 | ID NO: |
| CAR22-65 | GDSML | 288 | RTYHRST | 290 | VRLQDG | 291 |
| Combined | SNSDT | | WYDDYAS | | NSWSDA | |
| | WN | | SVRG | | FDV | |
| CAR22-65 | SNSDT | 289 | RTYHRST | 290 | VRLQDG | 291 |
| Kabat | WN | | SSVRG | | NSWSDA | |
| | | | WYDDYA | | FDV | |

TABLE 17
Light Chain Variable Domain CDRs of CD22 CAR
(CAR22-65). The LC CDR sequences
in this table have the same sequence under
the Kabat or combined definitions.

| | | SEQ | | SEQ | | SEQ |
|---|---|---|---|---|---|---|
| Candidate | LCDR1 | ID NO: | LCDR2 | ID NO: | LCDR3 | ID NO: |
| CAR22-65 | TGTSSDV | 95 | DVSNRPS | 96 | SSYTSS | 97 |
| Combined | GGYNYVS | | | | STLYV | |

In some embodiments, the antigen binding domain comprises a HC CDR1, a HC CDR2, and a HC CDR3 of any heavy chain binding domain amino acid sequences listed in Table 16. In embodiments, the antigen binding domain further comprises a LC CDR1, a LC CDR2, and a LC CDR3. In embodiments, the antigen binding domain comprises a LC CDR1, a LC CDR2, and a LC CDR3 amino acid sequences listed in Table 17.

In some embodiments, the antigen binding domain comprises one, two or all of LC CDR1, LC CDR2, and LC CDR3 of any light chain binding domain amino acid sequences listed in Table 17, and one, two or all of HC CDR1, HC CDR2, and HC CDR3 of any heavy chain binding domain amino acid sequences listed in Table 16.

In some embodiments, the CDRs are defined according to the Kabat numbering scheme, the Chothia numbering scheme, or a combination thereof.

The order in which the VL and VH domains appear in the scFv can be varied (i.e., VL-VH, or VH-VL orientation), and where any of one, two, three or four copies of the “G4S” subunit (SEQ ID NO: 25), in which each subunit comprises the sequence GGGGS (SEQ ID NO: 25) (for example, (G4S)₃ (SEQ ID NO: 28) or (G4S)₄ (SEQ ID NO: 27)), can connect the variable domains to create the entirety of the scFv domain. Alternatively, the CAR construct can include, for example, a linker including the sequence GSTSGSGKPGSGEGSTKG (SEQ ID NO: 43). Alternatively, the CAR construct can include, for example, a linker including the sequence LAEAAAK (SEQ ID NO: 308). In some embodiments, the CAR construct does not include a linker between the VL and VH domains.

These clones all contained a Q/K residue change in the signal domain of the co-stimulatory domain derived from CD3zeta chain.

### EGFR CAR

In some embodiments, the CAR-expressing cell described herein is an EGFR CAR-expressing cell (for example, a cell expressing a CAR that binds to human EGFR). In some embodiments, the CAR-expressing cell described herein is an EGFRvIII CAR-expressing cell (for example, a cell expressing a CAR that binds to human EGFRvIII). Exemplary EGFRvIII CARs can include sequences disclosed in WO2014/130657, for example, Table 2 of WO2014/130657, incorporated herein by reference.

Exemplary EGFRvIII-binding sequences or EGFR CAR sequences may comprise a CDR, a variable region, an scFv, or a full-length CAR sequence of a EGFR CAR disclosed in WO2014/130657.

### Mesothelin CAR

In some embodiments, the CAR-expressing cell described herein is a mesothelin CAR-expressing cell (for example, a cell expressing a CAR that binds to human mesothelin). Exemplary mesothelin CARs can include sequences disclosed in WO2015090230 and WO2017112741, for example, Tables 2, 3, 4, and 5 of WO2017112741, incorporated herein by reference.

### Other Exemplary CARs

In other embodiments, the CAR-expressing cells can specifically bind to CD123, for example, can include a CAR molecule (for example, any of the CAR1 to CAR8), or an antigen binding domain according to Tables 1-2 of WO 2014/130635, incorporated herein by reference. The amino acid and nucleotide sequences encoding the CD123 CAR molecules and antigen binding domains (for example, including one, two, three VH CDRs; and one, two, three VL CDRs according to Kabat or Chothia), are specified in WO 2014/130635. In other embodiments, the CAR-expressing cells can specifically bind to CD123, for example, can include a CAR molecule (for example, any of the CAR123-1 to CAR123-4 and hzCAR123-1 to hzCAR123-32), or an antigen binding domain according to Tables 2, 6, and 9 of WO2016/028896, incorporated herein by reference. The amino acid and nucleotide sequences encoding the CD123 CAR molecules and antigen binding domains (for example, including one, two, three VH CDRs; and one, two, three VL CDRs according to Kabat or Chothia), are specified in WO2016/028896.

In some embodiments, the CAR molecule comprises a CLL1 CAR described herein, for example, a CLL1 CAR described in US2016/0051651A1, incorporated herein by reference. In embodiments, the CLL1 CAR comprises an amino acid, or has a nucleotide sequence shown in US2016/0051651A1, incorporated herein by reference. In other embodiments, the CAR-expressing cells can specifically bind to CLL-1, for example, can include a CAR molecule, or an antigen binding domain according to Table 2 of WO2016/014535, incorporated herein by reference. The amino acid and nucleotide sequences encoding the CLL-1 CAR molecules and antigen binding domains (for example, including one, two, three VH CDRs; and one, two, three VL CDRs according to Kabat or Chothia), are specified in WO2016/014535.

In some embodiments, the CAR molecule comprises a CD33 CAR described herein, e.ga CD33 CAR described in US2016/0096892A1, incorporated herein by reference. In embodiments, the CD33 CAR comprises an amino acid, or has a nucleotide sequence shown in US2016/0096892A1, incorporated herein by reference. In other embodiments, the CAR-expressing cells can specifically bind to CD33, for example, can include a CAR molecule (for example, any of CAR33-1 to CAR-33-9), or an antigen binding domain according to Table 2 or 9 of WO2016/014576, incorporated herein by reference. The amino acid and nucleotide sequences encoding the CD33 CAR molecules and antigen binding domains (for example, including one, two, three VH CDRs; and one, two, three VL CDRs according to Kabat or Chothia), are specified in WO2016/014576.

In some embodiments, the antigen binding domain comprises one, two three (for example, all three) heavy chain CDRs, HC CDR1, HC CDR2 and HC CDR3, from an antibody described herein (for example, an antibody described in WO2015/142675, US-2015-0283178-Al, US-2016-0046724-A1, US2014/0322212A1, US2016/0068601A1, US2016/0051651A1, US2016/0096892A1, US2014/0322275A1, or WO2015/090230, incorporated herein by reference), and/or one, two, three (for example, all three) light chain CDRs, LC CDR1, LC CDR2 and LC CDR3, from an antibody described herein (for example, an antibody described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference). In some embodiments, the antigen binding domain comprises a heavy chain variable region and/or a variable light chain region of an antibody listed above.

In embodiments, the antigen binding domain is an antigen binding domain described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference.

In embodiments, the antigen binding domain targets BCMA and is described in US-2016-0046724-A1. In embodiments, the antigen binding domain targets CD19 and is described in US-2015-0283178-A1. In embodiments, the antigen binding domain targets CD123 and is described in US2014/0322212A1, US2016/0068601A1. In embodiments, the antigen binding domain targets CLL1 and is described in US2016/0051651A1. In embodiments, the antigen binding domain targets CD33 and is described in US2016/0096892A1.

Exemplary target antigens that can be targeted using the CAR-expressing cells, include, but are not limited to, CD19, CD123, EGFRvIII, CD33, mesothelin, BCMA, and GFR ALPHA-4, among others, as described in, for example, WO2014/153270, WO 2014/130635, WO2016/028896, WO 2014/130657, WO2016/014576, WO 2015/090230, WO2016/014565, WO2016/014535, and WO2016/025880, each of which is herein incorporated by reference in its entirety.

In other embodiments, the CAR-expressing cells can specifically bind to GFR ALPHA-4, for example, can include a CAR molecule, or an antigen binding domain according to Table 2 of WO2016/025880, incorporated herein by reference. The amino acid and nucleotide sequences encoding the GFR ALPHA-4 CAR molecules and antigen binding domains (for example, including one, two, three VH CDRs; and one, two, three VL CDRs according to Kabat or Chothia), are specified in WO2016/025880.

In some embodiments, the antigen binding domain of any of the CAR molecules described herein (for example, any of CD19, CD123, EGFRvIII, CD33, mesothelin, BCMA, and GFR ALPHA-4) comprises one, two three (for example, all three) heavy chain CDRs, HC CDR1, HC CDR2 and HC CDR3, from an antibody listed above, and/or one, two, three (for example, all three) light chain CDRs, LC CDR1, LC CDR2 and LC CDR3, from an antigen binding domain listed above. In some embodiments, the antigen binding domain comprises a heavy chain variable region and/or a variable light chain region of an antibody listed or described above.

In some embodiments, the antigen binding domain comprises one, two three (for example, all three) heavy chain CDRs, HC CDR1, HC CDR2 and HC CDR3, from an antibody listed above, and/or one, two, three (for example, all three) light chain CDRs, LC CDR1, LC CDR2 and LC CDR3, from an antibody listed above. In some embodiments, the antigen binding domain comprises a heavy chain variable region and/or a variable light chain region of an antibody listed or described above.

In some embodiments, the tumor antigen is a tumor antigen described in International Application WO2015/142675, filed Mar. 13, 2015, which is herein incorporated by reference in its entirety. In some embodiments, the tumor antigen is chosen from one or more of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGSS); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAXS); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

In some embodiments, the antigen binding domain comprises one, two three (for example, all three) heavy chain CDRs, HC CDR1, HC CDR2 and HC CDR3, from an antibody listed above, and/or one, two, three (for example, all three) light chain CDRs, LC CDR1, LC CDR2 and LC CDR3, from an antibody listed above. In some embodiments, the antigen binding domain comprises a heavy chain variable region and/or a variable light chain region of an antibody listed or described above.

In some embodiments, the anti-tumor antigen binding domain is a fragment, for example, a single chain variable fragment (scFv). In some embodiments, the anti-a cancer associate antigen as described herein binding domain is a Fv, a Fab, a (Fab)2, or a bi-functional (for example bi-specific) hybrid antibody (for example, Lanzavecchia et al., Eur. J. Immunol. 17, 105 (1987)). In some embodiments, the antibodies and fragments thereof of this disclosure binds a cancer associate antigen as described herein protein with wild-type or enhanced affinity.

In some instances, scFvs can be prepared according to a method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (for example, a Ser-Gly linker) with an optimized length and/or amino acid composition. The linker length can greatly affect how the variable regions of a scFv fold and interact. In fact, if a short polypeptide linker is employed (for example, between 5-10 amino acids) intrachain folding is prevented. Interchain folding is also required to bring the two variable regions together to form a functional epitope binding site. For examples of linker orientation and size see, for example, Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, which are incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In some embodiments, the linker sequence comprises sets of glycine and serine repeats such as (Gly₄Ser)n, where n is a positive integer equal to or greater than 1 (SEQ ID NO: 25). In some embodiments, the linker can be (Gly₄Ser)₄ (SEQ ID NO: 27) or (Gly₄Ser)₃(SEQ ID NO: 28). Variation in the linker length may retain or enhance activity, giving rise to superior efficacy in activity studies.

In some embodiments, the antigen binding domain is a T cell receptor (“TCR”), or a fragment thereof, for example, a single chain TCR (scTCR). Methods to make such TCRs are known in the art. See, for example, Willemsen R A et al, Gene Therapy 7: 1369-1377 (2000); Zhang T et al, Cancer Gene Ther 11: 487-496 (2004); Aggen et al, Gene Ther. 19(4):365-74 (2012) (references are incorporated herein by its entirety). For example, scTCR can be engineered that contains the Vα and Vβ genes from a T cell clone linked by a linker (for example, a flexible peptide). This approach is very useful to cancer associated target that itself is intracellular, however, a fragment of such antigen (peptide) is presented on the surface of the cancer cells by MHC.

### Transmembrane Domain

With respect to the transmembrane domain, in various embodiments, a CAR (e.g., a CCAR) can be designed to comprise a transmembrane domain that is attached to the extracellular domain of the CAR. A transmembrane domain can include one or more additional amino acids adjacent to the transmembrane region, for example, one or more amino acid associated with the extracellular region of the protein from which the transmembrane was derived (for example, 1, 2, 3, 4, 5, 6, 7, 8, 9, 10 up to 15 amino acids of the extracellular region) and/or one or more additional amino acids associated with the intracellular region of the protein from which the transmembrane protein is derived (for example, 1, 2, 3, 4, 5, 6, 7, 8, 9, 10 up to 15 amino acids of the intracellular region). In some embodiments, the transmembrane domain is one that is associated with one of the other domains of the CAR is used. In some instances, the transmembrane domain can be selected or modified by amino acid substitution to avoid binding of such domains to the transmembrane domains of the same or different surface membrane proteins, for example, to minimize interactions with other members of the receptor complex. In some embodiments, the transmembrane domain is capable of homodimerization with another CAR on the CAR-expressing cell, for example, CART cell, surface. In some embodiments the amino acid sequence of the transmembrane domain may be modified or substituted so as to minimize interactions with the binding domains of the native binding partner present in the same CAR-expressing cell, for example, CART.

The transmembrane domain may be derived either from a natural or from a recombinant source. Where the source is natural, the domain may be derived from any membrane-bound or transmembrane protein. In some embodiments the transmembrane domain is capable of signaling to the intracellular domain(s) whenever the CAR has bound to a target. A transmembrane domain of particular use in this disclosure may include at least the transmembrane region(s) of, for example, the alpha, beta or zeta chain of T-cell receptor, CD28, CD3 epsilon, CD45, CD4, CD5, CD8 (for example, CD8 alpha, CD8 beta), CD9, CD16, CD22, CD33, CD37, CD64, CD80, CD86, CD134, CD137, CD154. In some embodiments, a transmembrane domain may include at least the transmembrane region(s) of a costimulatory molecule, for example, MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CD5, ICAM-1, LFA-1 (CD11a/CD18), 4-1BB (CD137), B7-H3, CD5, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

In some instances, the transmembrane domain can be attached to the extracellular region of the CAR, for example, the antigen binding domain of the CAR, via a hinge, for example, a hinge from a human protein. For example, in some embodiments, the hinge can be a human Ig (immunoglobulin) hinge, for example, an IgG4 hinge, or a CD8a hinge. In some embodiments, the hinge or spacer comprises (for example, consists of) the amino acid sequence of SEQ ID NO: 2. In some embodiments, the transmembrane domain comprises (for example, consists of) a transmembrane domain of SEQ ID NO: 6.

In some embodiments, the hinge or spacer comprises an IgG4 hinge. For example, in some embodiments, the hinge or spacer comprises a hinge of SEQ ID NO: 3. In some embodiments, the hinge or spacer comprises a hinge encoded by the nucleotide sequence of SEQ ID NO: 14.

In some embodiments, the hinge or spacer comprises an IgD hinge. For example, in some embodiments, the hinge or spacer comprises a hinge of the amino acid sequence of SEQ ID NO: 4. In some embodiments, the hinge or spacer comprises a hinge encoded by the nucleotide sequence of SEQ ID NO:15.

In some embodiments, the transmembrane domain may be recombinant, in which case it will comprise predominantly hydrophobic residues such as leucine and valine. In some embodiments, a triplet of phenylalanine, tryptophan and valine can be found at each end of a recombinant transmembrane domain.

Optionally, a short oligo- or polypeptide linker, between 2 and 10 amino acids in length may form the linkage between the transmembrane domain and the cytoplasmic region of the CAR. A glycine-serine doublet provides a particularly suitable linker. For example, in some embodiments, the linker comprises the amino acid sequence of SEQ ID NO: 5. In some embodiments, the linker is encoded by a nucleotide sequence of SEQ ID NO: 16.

In some embodiments, the hinge or spacer comprises a KIR2DS2 hinge.

### Cytoplasmic Domain

The cytoplasmic domain or region of a CAR (e.g., a CCAR) of the present disclosure includes an intracellular signaling domain. An intracellular signaling domain is generally responsible for activation of at least one of the normal effector functions of the immune cell in which the CAR has been introduced.

Examples of intracellular signaling domains for use in the CAR of this disclosure include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, for example, a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM containing primary intracellular signaling domains that are of particular use in this disclosure include those of TCR zeta, FcR gamma, FcR beta, CD3 gamma, CD3 delta, CD3 epsilon, CD5, CD22, CD79a, CD79b, CD278 (also known as “ICOS”), FcεRI, DAP10, DAP12, and CD66d. In some embodiments, a CAR of this disclosure comprises an intracellular signaling domain, for example, a primary signaling domain of CD3-zeta.

In some embodiments, a primary signaling domain comprises a modified ITAM domain, for example, a mutated ITAM domain which has altered (for example, increased or decreased) activity as compared to the native ITAM domain. In some embodiments, a primary signaling domain comprises a modified ITAM-containing primary intracellular signaling domain, for example, an optimized and/or truncated ITAM-containing primary intracellular signaling domain. In some embodiments, a primary signaling domain comprises one, two, three, four or more ITAM motifs.

Further examples of molecules containing a primary intracellular signaling domain that are of particular use in this disclosure include those of DAP10, DAP12, and CD32.

The intracellular signaling domain of the CAR can comprise the primary signaling domain, for example, CD3-zeta signaling domain, by itself or it can be combined with any other desired intracellular signaling domain(s) useful in the context of a CAR of this disclosure. For example, the intracellular signaling domain of the CAR can comprise a primary signaling domain, for example, CD3 zeta chain portion, and a costimulatory signaling domain. The costimulatory signaling domain refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CD5, ICAM-1, LFA-1 (CD11a/CD18), 4-1BB (CD137), B7-H3, CD5, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83, and the like. For example, CD27 costimulation has been demonstrated to enhance expansion, effector function, and survival of human CART cells in vitro and augments human T cell persistence and antitumor activity in vivo (Song et al. Blood. 2012; 119(3):696-706). The intracellular signaling sequences within the cytoplasmic portion of the CAR of this disclosure may be linked to each other in a random or specified order. Optionally, a short oligo- or polypeptide linker, for example, between 2 and 10 amino acids (for example, 2, 3, 4, 5, 6, 7, 8, 9, or 10 amino acids) in length may form the linkage between intracellular signaling sequence. In some embodiments, a glycine-serine doublet can be used as a suitable linker. In some embodiments, a single amino acid, for example, an alanine, a glycine, can be used as a suitable linker.

In some embodiments, the intracellular signaling domain is designed to comprise two or more, for example, 2, 3, 4, 5, or more, costimulatory signaling domains. In some embodiments, the two or more, for example, 2, 3, 4, 5, or more, costimulatory signaling domains, are separated by a linker molecule, for example, a linker molecule described herein. In some embodiments, the intracellular signaling domain comprises two costimulatory signaling domains. In some embodiments, the linker molecule is a glycine residue. In some embodiments, the linker is an alanine residue.

In some embodiments, the intracellular signaling domain is designed to comprise the signaling domain of CD3-zeta and the signaling domain of CD28. In some embodiments, the intracellular signaling domain is designed to comprise the signaling domain of CD3-zeta and the signaling domain of 4-1BB. In some embodiments, the signaling domain of 4-1BB is a signaling domain of SEQ ID NO: 7. In some embodiments, the signaling domain of CD3-zeta is a signaling domain of SEQ ID NO: 9 (mutant CD3zeta) or SEQ ID NO: 10 (wild type human CD3zeta).

In some embodiments, the intracellular signaling domain is designed to comprise the signaling domain of CD3-zeta and the signaling domain of CD27. In some embodiments, the signaling domain of CD27 comprises the amino acid sequence of SEQ ID NO: 8. In some embodiments, the signaling domain of CD27 is encoded by the nucleic acid sequence of SEQ ID NO: 19.

In some embodiments, the intracellular is designed to comprise the signaling domain of CD3-zeta and the signaling domain of CD28. In some embodiments, the signaling domain of CD28 comprises the amino acid sequence of SEQ ID NO: 36. In some embodiments, the signaling domain of CD28 is encoded by the nucleic acid sequence of SEQ ID NO: 37.

In some embodiments, the intracellular is designed to comprise the signaling domain of CD3-zeta and the signaling domain of ICOS. In some embodiments, the signaling domain of ICOS comprises the amino acid sequence of SEQ ID NO: 38. In some embodiments, the signaling domain of ICOS is encoded by the nucleic acid sequence of SEQ ID NO: 39.

### CAR Configurations

### Dual CARs

In an embodiment, an immune cell (e.g., a T cell or NK cell) expresses two CARs, e.g., a first CAR that binds to a first antigen and a second CAR that binds to a second antigen. In an embodiment, the first antigen and the second antigen are different. In an embodiment, the first or second antigen is chosen from an antigen expressed on B cells, an antigen expressed on acute myeloid leukemia cells, or an antigen on solid tumor cells. In an embodiment, the first or second antigen is chosen from CD10, CD19, CD20, CD22, CD34, CD123, BCMA, FLT-3, ROR1, CD79b, CD179b, CD79a, CD34, CLL-1, folate receptor beta, FLT3, EGFRvIII, mesothelin, GD2, Tn antigen, sTn antigen, Tn-O-Glycopeptides, sTn-O-Glycopeptides, PSMA, CD97, TAG72, CD44v6, CEA, EPCAM, KIT, IL-13Ra2, leguman, GD3, CD171, IL-11Ra, PSCA, MAD-CT-1, MAD-CT-2, VEGFR2, LewisY, CD24, PDGFR-beta, SSEA-4, folate receptor alpha, ERBBs (e.g., ERBB2), Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, NY-ESO-1, GPR20, Ly6k, OR51E2, TARP, GFRα4, or a peptide of any of these antigens presented on MHC.

In an embodiment, the first CAR is encoded by a first nucleic acid sequence. In an embodiment, the second CAR is encoded by a second nucleic acid sequence. In an embodiment, the first and second nucleic acid sequences are disposed on a single nucleic acid molecule. In an embodiment, the first and second nucleic acid sequences are disposed on separate nucleic acid molecules. In an embodiment, the nucleic acid molecule or nucleic acid molecules are DNA or RNA molecules. In embodiments, the first and second nucleic acid sequences are situated in the same orientation, e.g., transcription of the first and second nucleic acid sequences proceeds in the same direction. In embodiments, the first and second nucleic acid sequences are situated in different orientations. In embodiments, a single promoter controls expression of the first and second nucleic acid sequences. In embodiments, a nucleic acid encoding a protease cleavage site (such as a T2A, P2A, E2A, or F2A cleavage site) is situated between the first and second nucleic acid sequences. In embodiments, the protease cleavage site is placed such that a cell can express a fusion protein comprising the first CAR and the second CAR and the fusion protein is subsequently processed into two peptides by proteolytic cleavage. In some embodiments, the first nucleic acid sequence is upstream of the second nucleic acid sequence, or the second nucleic acid sequence is upstream of the first nucleic acid sequence. In embodiments, a first promoter controls expression of the first nucleic acid sequence and a second promoter controls expression of the second nucleic acid sequence. In embodiments, the nucleic acid molecule is a plasmid. In embodiments, the nucleic acid molecule comprises a viral packaging element. In embodiments, the immune cell may comprise a protease (e.g., endogenous or exogenous protease) that cleaves a T2A, P2A, E2A, or F2A cleavage site.

In an embodiment, the first CAR comprises a first antigen-binding domain and the second CAR comprises a second antigen-binding domain. In an embodiment, the first or second antigen binding domain comprises a CDR, a VH, a VL, or a scFv disclosed herein, or an amino acid sequence having at least about 85%, 90%, 95%, or 99% sequence identity thereto.

### Multi-Specific CARs

In an embodiment, a CAR of this disclosure is a multi-specific CAR. In one embodiment, the multi-specific CAR is a bispecific CAR. In one embodiment, the bispecific CAR comprises an antigen binding domain which is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv, or fragment thereof, have binding specificity for a first epitope and a scFv, or fragment thereof, have binding specificity for a second epitope.

In some embodiments, a CAR of this disclosure comprises an antigen binding domain that is a multi-specific (e.g., a bispecific or a trispecific) antibody molecule. Protocols for generating bispecific or heterodimeric antibody molecules are known in the art; including but not limited to, for example, the “knob in a hole” approach described in, e.g., U.S. Pat. No. 5,731,168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bi-specific structure using a heterobifunctional reagent having an amine-reactive group and a sulfhydryl reactive group as described in, e.g., U.S. Pat. No. 4,433,059; bispecific antibody determinants generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies, e.g., three Fab fragments cross-linked through sulfhdryl reactive groups, as described in, e.g., U.S. Pat. No. 5,273,743; biosynthetic binding proteins, e.g., pair of scFvs cross-linked through C-terminal tails preferably through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono-and oligovalent receptors, e.g., VH-CH1 regions of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multispecific binding proteins, e.g., dimer of polypeptides having first domain with binding region of Ig heavy chain variable region, and second domain with binding region of Ig light chain variable region, generally termed diabodies (higher order structures are also encompassed creating for bispecifc, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VH and VL domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; String of VH domains (or VL domains in family members) connected by peptide linkages with crosslinkable groups at the C-terminus futher associated with VL domains to form a series of FVs (or scFvs), as described in, e.g., U.S. Pat. No. 5,864,019; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFV or diabody type format, as described in, e.g., U.S. Pat. No. 5,869,620. Additional exemplary multispecific and bispecific molecules and methods of making the same are found, for example, in U.S. Pat. Nos. 5,910,573, 5,932,448, 5,959,083, 5,989,830, 6,005,079, 6,239,259, 6,294,353, 6,333,396, 6,476,198, 6,511,663, 6,670,453, 6,743,896, 6,809,185, 6,833,441, 7,129,330, 7,183,076, 7,521,056, 7,527,787, 7,534,866, 7,612,181, US2002004587A1, US2002076406A1, US2002103345A1, US2003207346A1, US2003211078A1, US2004219643A1, US2004220388A1, US2004242847A1, US2005003403A1, US2005004352A1, US2005069552A1, US2005079170A1, US2005100543A1, US2005136049A1, US2005136051A1, US2005163782A1, US2005266425A1, US2006083747A1, US2006120960A1, US2006204493A1, US2006263367A1, US2007004909A1, US2007087381A1, US2007128150A1, US2007141049A1, US2007154901A1, US2007274985A1, US2008050370A1, US2008069820A1, US2008152645A1, US2008171855A1, US2008241884A1, US2008254512A1, US2008260738A1, US2009130106A1, US2009148905A1, US2009155275A1, US2009162359A1, US2009162360A1, US2009175851A1, US2009175867A1, US2009232811A1, US2009234105A1, US2009263392A1, US2009274649A1, EP346087A2, WO0006605A2, WO02072635A2, WO04081051A1, WO06020258A2, WO2007044887A2, WO2007095338A2, WO2007137760A2, WO2008119353A1, WO2009021754A2, WO2009068630A1, WO9103493A1, WO9323537A1, WO940913 1A1, WO9412625A2, WO9509917A1, WO9637621A2, WO9964460A1. The contents of the above-referenced applications are incorporated herein by reference in their entireties.

Within each antibody or antibody fragment (e.g., scFv) of a bispecific antibody molecule, the VH can be upstream or downstream of the VL. In some embodiments, the upstream antibody or antibody fragment (e.g., scFv) is arranged with its VH (VH₁) upstream of its VL (VL₁) and the downstream antibody or antibody fragment (e.g., scFv) is arranged with its VL (VL₂) upstream of its VH (VH₂), such that the overall bispecific antibody molecule has the arrangement VH₁-VL₁-VL₂-VH₂. In other embodiments, the upstream antibody or antibody fragment (e.g., scFv) is arranged with its VL (VL1) upstream of its VH (VH₁) and the downstream antibody or antibody fragment (e.g., scFv) is arranged with its VH (VH₂) upstream of its VL (VL₂), such that the overall bispecific antibody molecule has the arrangement VL₁-VH₁-VH₂-VL₂. Optionally, a linker is disposed between the two antibodies or antibody fragments (e.g., scFvs), e.g., between VL₁ and VL₂ if the construct is arranged as VH₁-VL₁-VL₂-VH₂, or between VH₁ and VH₂ if the construct is arranged as VL₁-VH₁-VH₂-VL₂. The linker may be a linker as described herein, e.g., a (Gly₄-Ser)n linker, wherein n is 1, 2, 3, 4, 5, or 6, preferably 4 (SEQ ID NO: 26). In general, the linker between the two scFvs should be long enough to avoid mispairing between the domains of the two scFvs. Optionally, a linker is disposed between the VL and VH of the first scFv. Optionally, a linker is disposed between the VL and VH of the second scFv. In constructs that have multiple linkers, any two or more of the linkers can be the same or different. Accordingly, in some embodiments, a bispecific CAR comprises VLs, VHs, and optionally one or more linkers in an arrangement as described herein.

### Diabody CAR

In some embodiments, a CAR of this disclosure is a bispecific CAR. In some embodiments, a CAR of this disclosure is a diabody CAR. In some embodiments, the diabody CAR comprises an antigen binding domain that binds to a first antigen and a second antigen. In some embodiments, the antigen binding domain comprises a VH1, a VL1, a VH2, and a VL2, wherein the VH1 and VL1 bind to the first antigen and the VH2 and VL2 bind to the second antigen. In some embodiments, the antigen binding domain has the arrangement VH1—optionally linker 1 (“L1”)-VH2—optionally linker 2 (“L2”)-VL2—optionally linker 3 (“L3”)-VL1 from the N-terminus to the C-terminus. In some embodiments, the antigen binding domain has the arrangement VH1—optionally L1-VL2—optionally L2-VH2—optionally L3-VL1 from the N-terminus to the C-terminus. In some embodiments, the antigen binding domain has the arrangement VL1—optionally L1-VH2—optionally L2-VL2 —optionally L3-VH1 from the N-terminus to the C-terminus. In some embodiments, the antigen binding domain has the arrangement VL1—optionally L1-VL2—optionally L2-VH2—optionally L3-VH1 from the N-terminus to the C-terminus. In some embodiments, the antigen binding domain has the arrangement VH2—optionally L1-VH1—optionally L2-VL1—optionally L3-VL2 from the N-terminus to the C-terminus. In some embodiments, the antigen binding domain has the arrangement VH2—optionally L1-VL1—optionally L2-VH1—optionally L3-VL2 from the N-terminus to the C-terminus. In some embodiments, the antigen binding domain has the arrangement VL2—optionally L1-VH1—optionally L2-VL1—optionally L3-VH2 from the N-terminus to the C-terminus. In some embodiments, the antigen binding domain has the arrangement VL2—optionally L1-VL1—optionally L2-VH1—optionally L3-VH2 from the N-terminus to the C-terminus. In some embodiments, the antigen binding domain has the arrangement VH1-linker 1 (“L1”)-VH2-linker 2 (“L2”)-VL2-linker 3 (“L3”)-VL1 from the N-terminus to the C-terminus. In some embodiments, the antigen binding domain has the arrangement VH1-L1-VL2-L2-VH2-L3-VL1 from the N-terminus to the C-terminus. In some embodiments, the antigen binding domain has the arrangement VL1-L1-VH2-L2-VL2-L3-VH1 from the N-terminus to the C-terminus. In some embodiments, the antigen binding domain has the arrangement VL1-L1-VL2-L2-VH2-L3-VH1 from the N-terminus to the C-terminus. In some embodiments, the antigen binding domain has the arrangement VH2-L1-VH1-L2-VL1-L3-VL2 from the N-terminus to the C-terminus. In some embodiments, the antigen binding domain has the arrangement VH2-L1-VL1-L2-VH1-L3-VL2 from the N-terminus to the C-terminus. In some embodiments, the antigen binding domain has the arrangement VL2-L1-VH1-L2-VL1-L3-VH2 from the N-terminus to the C-terminus. In some embodiments, the antigen binding domain has the arrangement VL2-L1-VL1-L2-VH1-L3-VH2 from the N-terminus to the C-terminus. In some embodiments, the variable regions are fused by a linker comprising the amino acid sequence of GGGGSGGGGS (SEQ ID NO: 5). In some embodiments, the variable regions are fused by a linker comprising the amino acid sequence of GGGGSGGGGSGGGGSGGGGS (SEQ ID NO: 63). In some embodiments, L1 comprises the amino acid sequence of SEQ ID NO: 5. In some embodiments, L2 comprises the amino acid sequence of SEQ ID NO: 63. In some embodiments, L3 comprises the amino acid sequence of SEQ ID NO: 5. In some embodiments, the VH1, VL1, VH2, or VL2 comprises a CDR, a VH, or a VL sequence disclosed herein, or an amino acid sequence having at least about 85%, 90%, 95%, or 99% sequence identity thereto. In some embodiments, a diabody disclosed herein comprises an engineered disulfide bridge, e.g., to stabilize the diabody and/or to facilitate correct pairing of the VH and VL. In some embodiments, the engineered disulfide bridge is between the variable region that is most proximal to the hinge region (e.g., the VH or VL region that is most proximal to the hinge region) and its corresponding pairing partner (e.g., the corresponding VL or the corresponding VH).

In some embodiments, the first antigen and the second antigen are different. In some embodiments, the first or second antigen is chosen from an antigen expressed on B cells, an antigen expressed on acute myeloid leukemia cells, or an antigen on solid tumor cells. In some embodiments, the first or second antigen is chosen from CD10, CD19, CD20, CD22, CD34, CD123, BCMA, FLT-3, ROR1, CD79b, CD179b, CD79a, CD34, CLL-1, folate receptor beta, FLT3, EGFRvIII, mesothelin, GD2, Tn antigen, sTn antigen, Tn-O-Glycopeptides, sTn-O-Glycopeptides, PSMA, CD97, TAG72, CD44v6, CEA, EPCAM, KIT, IL-13Ra2, leguman, GD3, CD171, IL-11Ra, PSCA, MAD-CT-1, MAD-CT-2, VEGFR2, LewisY, CD24, PDGFR-beta, SSEA-4, folate receptor alpha, ERBBs (e.g., ERBB2), Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, NY-ESO-1, GPR20, Ly6k, OR51E2, TARP, GFRα4, or a peptide of any of these antigens presented on MHC.

### Chimeric TCR

In one aspect, the antibodies and antibody fragments of the present disclosure can be grafted to one or more constant domain of a T cell receptor (“TCR”) chain, for example, a TCR alpha or TCR beta chain, to create a chimeric TCR. Without being bound by theory, it is believed that chimeric TCRs will signal through the TCR complex upon antigen binding. For example, a scFv as disclosed herein, can be grafted to the constant domain, e.g., at least a portion of the extracellular constant domain, the transmembrane domain and the cytoplasmic domain, of a TCR chain, for example, the TCR alpha chain and/or the TCR beta chain. As another example, an antibody fragment, for example a VL domain as described herein, can be grafted to the constant domain of a TCR alpha chain, and an antibody fragment, for example a VH domain as described herein, can be grafted to the constant domain of a TCR beta chain (or alternatively, a VL domain may be grafted to the constant domain of the TCR beta chain and a VH domain may be grafted to a TCR alpha chain). As another example, the CDRs of an antibody or antibody fragment, e.g., the CDRs of an antibody or antibody fragment as described herein may be grafted into a TCR alpha and/or beta chain to create a chimeric TCR. For example, the LCDRs disclosed herein may be grafted into the variable domain of a TCR alpha chain and the HCDRs disclosed herein may be grafted to the variable domain of a TCR beta chain, or vice versa. Such chimeric TCRs may be produced by methods known in the art (For example, Willemsen R A et al, Gene Therapy 2000; 7: 1369-1377; Zhang T et al, Cancer Gene Ther 2004; 11: 487-496; Aggen et al, Gene Ther. 2012 April; 19(4):365-74).

### Additional Embodiments

In one embodiment, when the CAR-expressing cell comprises two or more different CARs, the antigen binding domains of the different CARs can be such that the antigen binding domains do not interact with one another. For example, a cell expressing a first and second CAR can have an antigen binding domain of the first CAR, e.g., as a fragment, e.g., an scFv, that does not form an association with the antigen binding domain of the second CAR, e.g., the antigen binding domain of the second CAR is a VHH.

In some embodiments, the antigen binding domain comprises a single domain antigen binding (SDAB) molecules include molecules whose complementary determining regions are part of a single domain polypeptide. Examples include, but are not limited to, heavy chain variable domains, binding molecules naturally devoid of light chains, single domains derived from conventional 4-chain antibodies, engineered domains and single domain scaffolds other than those derived from antibodies. SDAB molecules may be any of the art, or any future single domain molecules. SDAB molecules may be derived from any species including, but not limited to mouse, human, camel, llama, lamprey, fish, shark, goat, rabbit, and bovine. This term also includes naturally occurring single domain antibody molecules from species other than Camelidae and sharks.

In one aspect, an SDAB molecule can be derived from a variable region of the immunoglobulin found in fish, such as, for example, that which is derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov (2005) Protein Sci. 14:2901-2909.

According to another aspect, an SDAB molecule is a naturally occurring single domain antigen binding molecule known as heavy chain devoid of light chains. Such single domain molecules are disclosed in WO 9404678 and Hamers-Casterman, C. et al. (1993) Nature 363:446-448, for example. For clarity reasons, this variable domain derived from a heavy chain molecule naturally devoid of light chain is known herein as a VHH or nanobody to distinguish it from the conventional VH of four chain immunoglobulins. Such a VHH molecule can be derived from Camelidae species, for example in camel, llama, dromedary, alpaca and guanaco. Other species besides Camelidae may produce heavy chain molecules naturally devoid of light chain; such VHHs are within the scope of this disclosure.

The SDAB molecules can be recombinant, CDR-grafted, humanized, camelized, de-immunized and/or in vitro generated (e.g., selected by phage display).

It has also been discovered, that cells having a plurality of chimeric membrane embedded receptors comprising an antigen binding domain that interactions between the antigen binding domain of the receptors can be undesirable, e.g., because it inhibits the ability of one or more of the antigen binding domains to bind its cognate antigen. Accordingly, disclosed herein are cells having a first and a second non-naturally occurring chimeric membrane embedded receptor comprising antigen binding domains that minimize such interactions. Also disclosed herein are nucleic acids encoding a first and a second non-naturally occurring chimeric membrane embedded receptor comprising antigen binding domains that minimize such interactions, as well as methods of making and using such cells and nucleic acids. In an embodiment the antigen binding domain of one of said first said second non-naturally occurring chimeric membrane embedded receptor, comprises an scFv, and the other comprises a single VH domain, e.g., a camelid, shark, or lamprey single VH domain, or a single VH domain derived from a human or mouse sequence.

In some embodiments, this disclosure comprises a first and second CAR, wherein the antigen binding domain of one of said first CAR said second CAR does not comprise a variable light domain and a variable heavy domain. In some embodiments, the antigen binding domain of one of said first CAR said second CAR is an scFv, and the other is not an scFv. In some embodiments, the antigen binding domain of one of said first CAR said second CAR comprises a single VH domain, e.g., a camelid, shark, or lamprey single VH domain, or a single VH domain derived from a human or mouse sequence. In some embodiments, the antigen binding domain of one of said first CAR said second CAR comprises a nanobody. In some embodiments, the antigen binding domain of one of said first CAR said second CAR comprises a camelid VHH domain.

In some embodiments, the antigen binding domain of one of said first CAR said second CAR comprises an scFv, and the other comprises a single VH domain, e.g., a camelid, shark, or lamprey single VH domain, or a single VH domain derived from a human or mouse sequence. In some embodiments, the antigen binding domain of one of said first CAR said second CAR comprises an scFv, and the other comprises a nanobody. In some embodiments, the antigen binding domain of one of said first CAR said second CAR comprises an scFv, and the other comprises a camelid VHH domain.

In some embodiments, when present on the surface of a cell, binding of the antigen binding domain of said first CAR to its cognate antigen is not substantially reduced by the presence of said second CAR. In some embodiments, binding of the antigen binding domain of said first CAR to its cognate antigen in the presence of said second CAR is 85%, 90%, 95%, 96%, 97%, 98% or 99% of binding of the antigen binding domain of said first CAR to its cognate antigen in the absence of said second CAR.

In some embodiments, when present on the surface of a cell, the antigen binding domains of said first CAR said second CAR, associate with one another less than if both were scFv antigen binding domains. In some embodiments, the antigen binding domains of said first CAR said second CAR, associate with one another 85%, 90%, 95%, 96%, 97%, 98% or 99% less than if both were scFv antigen binding domains.

### Natural Killer Cell Receptor (NKR) CARs

In an embodiment, the CAR molecule described herein comprises one or more components of a natural killer cell receptor (NKR), thereby forming an NKR-CAR. The NKR component can be a transmembrane domain, a hinge domain, or a cytoplasmic domain from any of the following natural killer cell receptors: killer cell immunoglobulin-like receptor (KIR), e.g., KIR2DL1, KIR2DL2/L3, KIR2DL4, KIR2DL5A, KIR2DL5B, KIR2DS1, KIR2DS2, KIR2DS3, KIR2DS4, DIR2DS5, KIR3DL1/S1, KIR3DL2, KIR3DL3, KIR2DP1, and KIR3DP1; natural cytotoxicity receptor (NCR), e.g., NKp30, NKp44, NKp46; signaling lymphocyte activation molecule (SLAM) family of immune cell receptors, e.g., CD48, CD229, 2B4, CD84, NTB-A, CRACC, BLAME, and CD2F-10; Fc receptor (FcR), e.g., CD16, and CD64; and Ly49 receptors, e.g., LY49A, LY49C. The NKR-CAR molecules described herein may interact with an adaptor molecule or intracellular signaling domain, e.g., DAP12. Exemplary configurations and sequences of CAR molecules comprising NKR components are described in International Publication No. WO2014/145252, the contents of which are hereby incorporated by reference.

### Non-Antibody Scaffolds

In embodiments, the antigen binding domain comprises a non-antibody scaffold, for example, a fibronectin, ankyrin, domain antibody, lipocalin, small modular immuno-pharmaceutical, maxybody, Protein A, or affilin. The non-antibody scaffold has the ability to bind to target antigen on a cell. In embodiments, the antigen binding domain is a polypeptide or fragment thereof of a naturally occurring protein expressed on a cell. In some embodiments, the antigen binding domain comprises a non-antibody scaffold. A wide variety of non-antibody scaffolds can be employed so long as the resulting polypeptide includes at least one binding region which specifically binds to the target antigen on a target cell.

Non-antibody scaffolds include: fibronectin (Novartis, Mass.), ankyrin (Molecular Partners AG, Zurich, Switzerland), domain antibodies (Domantis, Ltd., Cambridge, Mass., and Ablynx nv, Zwijnaarde, Belgium), lipocalin (Pieris Proteolab AG, Freising, Germany), small modular immuno-pharmaceuticals (Trubion Pharmaceuticals Inc., Seattle, Wash.), maxybodies (Avidia, Inc., Mountain View, Calif.), Protein A (Affibody AG, Sweden), and affilin (gamma-crystallin or ubiquitin) (Scil Proteins GmbH, Halle, Germany).

### Split CAR

In some embodiments, the CAR-expressing cell uses a split CAR. The split CAR approach is described in more detail in publications WO2014/055442 and WO2014/055657, incorporated herein by reference. Briefly, a split CAR system comprises a cell expressing a first CAR having a first antigen binding domain and a costimulatory domain (e.g., 41BB), and the cell also expresses a second CAR having a second antigen binding domain and an intracellular signaling domain (e.g., CD3 zeta). When the cell encounters the first antigen, the costimulatory domain is activated, and the cell proliferates. When the cell encounters the second antigen, the intracellular signaling domain is activated and cell-killing activity begins. Thus, the CAR-expressing cell is only fully activated in the presence of both antigens. In embodiments the first antigen binding domain recognizes BCMA, e.g., comprises an antigen binding domain described herein, and the second antigen binding domain recognizes an antigen expressed on acute myeloid leukemia cells, e.g., CD123, CLL-1, CD34, FLT3, or folate receptor beta. In embodiments the first antigen binding domain recognizes BCMA, e.g., comprises an antigen binding domain described herein, and the second antigen binding domain recognizes an antigen expressed on B-cells, e.g., CD10, CD19, CD20, CD22, CD34, CD123, FLT-3, ROR1, CD79b, CD179b, or CD79a.

Co-Expression of CAR with Other Molecules or Agents

Co-Expression of a Second CAR

In some embodiments, the CAR-expressing cell described herein can further comprise a second CAR, for example, a second CAR that includes a different antigen binding domain, for example, to the same target (for example, CD19) or a different target (for example, a target other than CD19, for example, a target described herein). In some embodiments, the CAR-expressing cell comprises a first CAR that targets a first antigen and includes an intracellular signaling domain having a costimulatory signaling domain but not a primary signaling domain, and a second CAR that targets a second, different, antigen and includes an intracellular signaling domain having a primary signaling domain but not a costimulatory signaling domain. Placement of a costimulatory signaling domain, for example, 4-1BB, CD28, CD27, OX-40 or ICOS, onto the first CAR, and the primary signaling domain, for example, CD3 zeta, on the second CAR can limit the CAR activity to cells where both targets are expressed. In some embodiments, the CAR expressing cell comprises a first CAR that includes an antigen binding domain, a transmembrane domain and a costimulatory domain and a second CAR that targets another antigen and includes an antigen binding domain, a transmembrane domain and a primary signaling domain. In some embodiments, the CAR expressing cell comprises a first CAR that includes an antigen binding domain, a transmembrane domain and a primary signaling domain and a second CAR that targets another antigen and includes an antigen binding domain to the antigen, a transmembrane domain and a costimulatory signaling domain.

In some embodiments, the CAR-expressing cell comprises an XCAR described herein and an inhibitory CAR. In some embodiments, the inhibitory CAR comprises an antigen binding domain that binds an antigen found on normal cells but not cancer cells, for example, normal cells that also express X. In some embodiments, the inhibitory CAR comprises the antigen binding domain, a transmembrane domain and an intracellular domain of an inhibitory molecule. For example, the intracellular domain of the inhibitory CAR can be an intracellular domain of PD1, PD-L1, PD-L2, CTLA4, TIM3, CEACAM (CEACAM-1, CEACAM-3, and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF (for example, TGF beta).

In some embodiments, when the CAR-expressing cell comprises two or more different CARs, the antigen binding domains of the different CARs can be such that the antigen binding domains do not interact with one another. For example, a cell expressing a first and second CAR can have an antigen binding domain of the first CAR, for example, as a fragment, for example, an scFv, that does not form an association with the antigen binding domain of the second CAR, for example, the antigen binding domain of the second CAR is a VHH.

In some embodiments, the antigen binding domain comprises a single domain antigen binding (SDAB) molecules include molecules whose complementary determining regions are part of a single domain polypeptide. Examples include, but are not limited to, heavy chain variable domains, binding molecules naturally devoid of light chains, single domains derived from conventional 4-chain antibodies, engineered domains and single domain scaffolds other than those derived from antibodies. SDAB molecules may be any of the art, or any future single domain molecules. SDAB molecules may be derived from any species including, but not limited to mouse, human, camel, llama, lamprey, fish, shark, goat, rabbit, and bovine. This term also includes naturally occurring single domain antibody molecules from species other than Camelidae and sharks.

In some embodiments, an SDAB molecule can be derived from a variable region of the immunoglobulin found in fish, such as, for example, that which is derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov (2005) Protein Sci. 14:2901-2909.

In some embodiments, an SDAB molecule is a naturally occurring single domain antigen binding molecule known as heavy chain devoid of light chains. Such single domain molecules are disclosed in WO 9404678 and Hamers-Casterman, C. et al. (1993) Nature 363:446-448, for example. For clarity reasons, this variable domain derived from a heavy chain molecule naturally devoid of light chain is known herein as a VHH or nanobody to distinguish it from the conventional VH of four chain immunoglobulins. Such a VHH molecule can be derived from Camelidae species, for example in camel, llama, dromedary, alpaca and guanaco. Other species besides Camelidae may produce heavy chain molecules naturally devoid of light chain; such VHHs are within the scope of this disclosure.

The SDAB molecules can be recombinant, CDR-grafted, humanized, camelized, de-immunized and/or in vitro generated (for example, selected by phage display).

It has also been discovered, that cells having a plurality of chimeric membrane embedded receptors comprising an antigen binding domain that interactions between the antigen binding domain of the receptors can be undesirable, for example, because it inhibits the ability of one or more of the antigen binding domains to bind its cognate antigen. Accordingly, disclosed herein are cells having a first and a second non-naturally occurring chimeric membrane embedded receptor comprising antigen binding domains that minimize such interactions. Also disclosed herein are nucleic acids encoding a first and a second non-naturally occurring chimeric membrane embedded receptor comprising an antigen binding domains that minimize such interactions, as well as methods of making and using such cells and nucleic acids. In some embodiments the antigen binding domain of one of the first and the second non-naturally occurring chimeric membrane embedded receptor, comprises an scFv, and the other comprises a single VH domain, for example, a camelid, shark, or lamprey single VH domain, or a single VH domain derived from a human or mouse sequence.

In some embodiments, a composition herein comprises a first and second CAR, wherein the antigen binding domain of one of the first and the second CAR does not comprise a variable light domain and a variable heavy domain. In some embodiments, the antigen binding domain of one of the first and the second CAR is an scFv, and the other is not an scFv. In some embodiments, the antigen binding domain of one of the first and the second CAR comprises a single VH domain, for example, a camelid, shark, or lamprey single VH domain, or a single VH domain derived from a human or mouse sequence. In some embodiments, the antigen binding domain of one of the first and the second CAR comprises a nanobody. In some embodiments, the antigen binding domain of one of the first and the second CAR comprises a camelid VHH domain.

In some embodiments, the antigen binding domain of one of the first and the second CAR comprises an scFv, and the other comprises a single VH domain, for example, a camelid, shark, or lamprey single VH domain, or a single VH domain derived from a human or mouse sequence. In some embodiments, the antigen binding domain of one of the first and the second CAR comprises an scFv, and the other comprises a nanobody. In some embodiments, the antigen binding domain of one of the first and the second CAR comprises an scFv, and the other comprises a camelid VHH domain.

In some embodiments, when present on the surface of a cell, binding of the antigen binding domain of the first CAR to its cognate antigen is not substantially reduced by the presence of the second CAR. In some embodiments, binding of the antigen binding domain of the first CAR to its cognate antigen in the presence of the second CAR is at least 85%, 90%, 95%, 96%, 97%, 98% or 99%, for example, 85%, 90%, 95%, 96%, 97%, 98% or 99% of binding of the antigen binding domain of the first CAR to its cognate antigen in the absence of the second CAR.

In some embodiments, when present on the surface of a cell, the antigen binding domains of the first and the second CAR, associate with one another less than if both were scFv antigen binding domains. In some embodiments, the antigen binding domains of the first and the second CAR, associate with one another at least 85%, 90%, 95%, 96%, 97%, 98% or 99% less than, for example, 85%, 90%, 95%, 96%, 97%, 98% or 99% less than if both were scFv antigen binding domains.

Co-Expression of an Agent that Enhances CAR Activity

In some embodiments, the CAR-expressing cell described herein can further express another agent, for example, an agent that enhances the activity or fitness of a CAR-expressing cell.

For example, in some embodiments, the agent can be an agent which inhibits a molecule that modulates or regulates, for example, inhibits, T cell function. In some embodiments, the molecule that modulates or regulates T cell function is an inhibitory molecule. Inhibitory molecules, for example, PD1, can, in some embodiments, decrease the ability of a CAR-expressing cell to mount an immune effector response. Examples of inhibitory molecules include PD1, PD-L1, CTLA4, TIM3, LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, or TGF beta.

In embodiments, an agent, for example, an inhibitory nucleic acid, for example, a dsRNA, for example, an siRNA or shRNA; or for example, an inhibitory protein or system, for example, a clustered regularly interspaced short palindromic repeats (CRISPR), a transcription-activator like effector nuclease (TALEN), or a zinc finger endonuclease (ZFN), for example, as described herein, can be used to inhibit expression of a molecule that modulates or regulates, for example, inhibits, T-cell function in the CAR-expressing cell. In some embodiments the agent is an shRNA, for example, an shRNA described herein. In some embodiments, the agent that modulates or regulates, for example, inhibits, T-cell function is inhibited within a CAR-expressing cell. For example, a dsRNA molecule that inhibits expression of a molecule that modulates or regulates, for example, inhibits, T-cell function is linked to the nucleic acid that encodes a component, for example, all of the components, of the CAR.

In some embodiments, the agent which inhibits an inhibitory molecule comprises a first polypeptide, for example, an inhibitory molecule, associated with a second polypeptide that provides a positive signal to the cell, for example, an intracellular signaling domain described herein. In some embodiments, the agent comprises a first polypeptide, for example, of an inhibitory molecule such as PD1, PD-L1, CTLA4, TIM3, LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, or TGF beta, or a fragment of any of these (for example, at least a portion of an extracellular domain of any of these), and a second polypeptide which is an intracellular signaling domain described herein (for example, comprising a costimulatory domain (for example, 41BB, CD27 or CD28, for example, as described herein) and/or a primary signaling domain (for example, a CD3 zeta signaling domain described herein). In some embodiments, the agent comprises a first polypeptide of PD1 or a fragment thereof (for example, at least a portion of an extracellular domain of PD1), and a second polypeptide of an intracellular signaling domain described herein (for example, a CD28 signaling domain described herein and/or a CD3 zeta signaling domain described herein). PD1 is an inhibitory member of the CD28 family of receptors that also includes CD28, CTLA-4, ICOS, and BTLA. PD-1 is expressed on activated B cells, T cells and myeloid cells (Agata et al. 1996 Int. Immunol 8:765-75). Two ligands for PD1, PD-L1 and PD-L2 have been shown to downregulate T cell activation upon binding to PD1 (Freeman et a. 2000 J Exp Med 192:1027-34; Latchman et al. 2001 Nat Immunol 2:261-8; Carter et al. 2002 Eur J Immunol 32:634-43). PD-L1 is abundant in human cancers (Dong et al. 2003 J Mol Med 81:281-7; Blank et al. 2005 Cancer Immunol. Immunother 54:307-314; Konishi et al. 2004 Clin Cancer Res 10:5094). Immune suppression can be reversed by inhibiting the local interaction of PD1 with PD-L1.

In some embodiments, the agent comprises the extracellular domain (ECD) of an inhibitory molecule, for example, Programmed Death 1 (PD1), can be fused to a transmembrane domain and intracellular signaling domains such as 41BB and CD3 zeta (also referred to herein as a PD1 CAR). In some embodiments, the PD1 CAR, when used in combinations with an XCAR described herein, improves the persistence of the T cell. In some embodiments, the CAR is a PD1 CAR comprising the extracellular domain of PD1 indicated as underlined in SEQ ID NO: 24. In some embodiments, the PD1 CAR comprises the amino acid sequence of SEQ ID NO: 24.

In some embodiments, the PD1 CAR comprises the amino acid sequence of SEQ ID NO: 22.

In some embodiments, the agent comprises a nucleic acid sequence encoding the PD1 CAR, for example, the PD1 CAR described herein. In some embodiments, the nucleic acid sequence for the PD1 CAR is provided as SEQ ID NO: 23, with the PD1 ECD underlined.

In another example, in some embodiments, the agent which enhances the activity of a CAR-expressing cell can be a costimulatory molecule or costimulatory molecule ligand. Examples of costimulatory molecules include MHC class I molecule, BTLA and a Toll ligand receptor, as well as OX40, CD27, CD28, CD5, ICAM-1, LFA-1 (CD11a/CD18), ICOS (CD278), and 4-1BB (CD137). Further examples of such costimulatory molecules include CD5, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83, for example, as described herein. Examples of costimulatory molecule ligands include CD80, CD86, CD40L, ICOSL, CD70, OX40L, 4-1BBL, GITRL, and LIGHT. In embodiments, the costimulatory molecule ligand is a ligand for a costimulatory molecule different from the costimulatory molecule domain of the CAR. In embodiments, the costimulatory molecule ligand is a ligand for a costimulatory molecule that is the same as the costimulatory molecule domain of the CAR. In some embodiments, the costimulatory molecule ligand is 4-1BBL. In some embodiments, the costimulatory ligand is CD80 or CD86. In some embodiments, the costimulatory molecule ligand is CD70. In embodiments, a CAR-expressing immune effector cell described herein can be further engineered to express one or more additional costimulatory molecules or costimulatory molecule ligands.

Co-Expression of CAR with a Chemokine Receptor

In embodiments, the CAR-expressing cell described herein, for example, CD19 CAR-expressing cell, further comprises a chemokine receptor molecule. Transgenic expression of chemokine receptors CCR2b or CXCR2 in T cells enhances trafficking to CCL2- or CXCL1-secreting solid tumors including melanoma and neuroblastoma (Craddock et al., J Immunother. 2010 October; 33(8):780-8 and Kershaw et al., Hum Gene Ther. 2002 Nov. 1; 13(16):1971-80). Thus, without wishing to be bound by theory, it is believed that chemokine receptors expressed in CAR-expressing cells that recognize chemokines secreted by tumors, for example, solid tumors, can improve homing of the CAR-expressing cell to the tumor, facilitate the infiltration of the CAR-expressing cell to the tumor, and enhances antitumor efficacy of the CAR-expressing cell. The chemokine receptor molecule can comprise a naturally occurring or recombinant chemokine receptor or a chemokine-binding fragment thereof. A chemokine receptor molecule suitable for expression in a CAR-expressing cell (for example, CAR-Tx) described herein include a CXC chemokine receptor (for example, CXCR1, CXCR2, CXCR3, CXCR4, CXCRS, CXCR6, or CXCR7), a CC chemokine receptor (for example, CCR1, CCR2, CCR3, CCR4, CCRS, CCR6, CCR7, CCR8, CCR9, CCR10, or CCR11), a CX3C chemokine receptor (for example, CX3CR1), a XC chemokine receptor (for example, XCR1), or a chemokine-binding fragment thereof. In some embodiments, the chemokine receptor molecule to be expressed with a CAR described herein is selected based on the chemokine(s) secreted by the tumor. In some embodiments, the CAR-expressing cell described herein further comprises, for example, expresses, a CCR2b receptor or a CXCR2 receptor. In some embodiments, the CAR described herein and the chemokine receptor molecule are on the same vector or are on two different vectors. In embodiments where the CAR described herein and the chemokine receptor molecule are on the same vector, the CAR and the chemokine receptor molecule are each under control of two different promoters or are under the control of the same promoter.

### Nucleic Acid Constructs Encoding a CAR

The present disclosure also provides an immune effector cell, for example, made by a method described herein, that includes a nucleic acid molecule encoding one or more CAR constructs (e.g., one or more CCAR constructs) described herein. In some embodiments, the nucleic acid molecule is provided as a messenger RNA transcript. In some embodiments, the nucleic acid molecule is provided as a DNA construct.

The nucleic acid molecules described herein can be a DNA molecule, an RNA molecule, or a combination thereof. In some embodiments, the nucleic acid molecule is an mRNA encoding a CAR polypeptide as described herein. In other embodiments, the nucleic acid molecule is a vector that includes any of the aforesaid nucleic acid molecules.

In some embodiments, the antigen binding domain of a CAR of this disclosure (for example, a scFv) is encoded by a nucleic acid molecule whose sequence has been codon optimized for expression in a mammalian cell. In some embodiments, entire CAR construct of this disclosure is encoded by a nucleic acid molecule whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, for example, methods disclosed in at least U.S. Pat. Nos. 5,786,464 and 6,114,148.

Accordingly, in some embodiments, an immune effector cell, for example, made by a method described herein, includes a nucleic acid molecule encoding a chimeric antigen receptor (CAR), wherein the CAR comprises an antigen binding domain that binds to a tumor antigen described herein, a transmembrane domain (for example, a transmembrane domain described herein), and an intracellular signaling domain (for example, an intracellular signaling domain described herein) comprising a stimulatory domain, for example, a costimulatory signaling domain (for example, a costimulatory signaling domain described herein) and/or a primary signaling domain (for example, a primary signaling domain described herein, for example, a zeta chain described herein).

The present disclosure also provides vectors in which a nucleic acid molecule encoding a CAR, for example, a nucleic acid molecule described herein, is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A retroviral vector may also be, for example, a gammaretroviral vector. A gammaretroviral vector may include, for example, a promoter, a packaging signal (w), a primer binding site (PBS), one or more (for example, two) long terminal repeats (LTR), and a transgene of interest, for example, a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, for example, in Tobias Maetzig et al., “Gammaretroviral Vectors: Biology, Technology and Application” Viruses. 2011 June; 3(6): 677-713.

In some embodiments, the vector comprising the nucleic acid encoding the desired CAR is an adenoviral vector (A5/35). In some embodiments, the expression of nucleic acids encoding CARs can be accomplished using of transposons such as sleeping beauty, crisper, CAS9, and zinc finger nucleases. See below June et al. 2009 Nature Reviews Immunology 9.10: 704-716, is incorporated herein by reference.

In brief summary, the expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (for example, WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used.

Additional promoter elements, for example, enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription. Exemplary promoters include the CMV IE gene, EF-1α, ubiquitin C, or phosphoglycerokinase (PGK) promoters.

An example of a promoter that is capable of expressing a CAR encoding nucleic acid molecule in a mammalian T cell is the EF1α promoter. The native EF1α promoter drives expression of the alpha subunit of the elongation factor-1 complex, which is responsible for the enzymatic delivery of aminoacyl tRNAs to the ribosome. The EF1apromoter has been extensively used in mammalian expression plasmids and has been shown to be effective in driving CAR expression from nucleic acid molecules cloned into a lentiviral vector. See, for example, Milone et al., Mol. Ther. 17(8): 1453-1464 (2009). In some embodiments, the EF1a promoter comprises the sequence provided in the Examples.

Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1α promoter, the hemoglobin promoter, and the creatine kinase promoter. Further, this disclosure should not be limited to the use of constitutive promoters. Inducible promoters are also contemplated as part of this disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Another example of a promoter is the phosphoglycerate kinase (PGK) promoter. In embodiments, a truncated PGK promoter (for example, a PGK promoter with one or more, for example, 1, 2, 5, 10, 100, 200, 300, or 400, nucleotide deletions when compared to the wild-type PGK promoter sequence) may be desired.

The nucleotide sequences of exemplary PCTK promoters are provided below.

WT PGK Promoter:
(SEQ ID NO: 190)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG
TTCCTTGGAAGGGCTGAATCCCCGCCTCGTCCTTCGCAGCGGCCCCCCGG
GTGTTCCCATCGCCGCTTCTAGGCCCACTGCGACGCTTGCCTGCACTTCT
TACACGCTCTGGGTCCCAGCCGCGGCGACGCAAAGGGCCTTGGTGCGGGT
CTCGTCGGCGCAGGGACGCGTTTGGGTCCCGACGGAACCTTTTCCGCGTT
GGGGTTGGGGCACCATAAGCT
Exemplary truncated PGK Promoters:
PGK 100:
(SEQ ID NO: 198)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTG
PGK200:
(SEQ ID NO: 191)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACG
PGK300:
(SEQ ID NO: 192)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG
TTCCTTGGAAGGGCTGAATCCCCG
PGK400:
(SEQ ID NO: 193)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG

TTCCTTGGAAGGGCTGAATCCCCGCCTCGTCCTTCGCAGCGGCCCCCCGG
GTGTTCCCATCGCCGCTTCTAGGCCCACTGCGACGCTTGCCTGCACTTCT
TACACGCTCTGGGTCCCAGCCG

A vector may also include, for example, a signal sequence to facilitate secretion, a polyadenylation signal and transcription terminator (for example, from Bovine Growth Hormone (BGH) gene), an element allowing episomal replication and replication in prokaryotes (for example SV40 origin and ColE1 or others known in the art) and/or elements to allow selection (for example, ampicillin resistance gene and/or zeocin marker).

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In some embodiments, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, for example, enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (for example, Ui-Tei et al., 2000 FEBS Letters 479: 79-82). Suitable expression systems are well known and may be prepared using known techniques or obtained commercially. In general, the construct with the minimal 5 flanking region showing the highest level of expression of reporter gene is identified as the promoter. Such promoter regions may be linked to a reporter gene and used to evaluate agents for the ability to modulate promoter-driven transcription.

In embodiments, the vector may comprise two or more nucleic acid sequences encoding a CAR, for example, a CAR described herein, for example, a CD19 CAR, and a second CAR, for example, an inhibitory CAR or a CAR that specifically binds to an antigen other than CD19. In such embodiments, the two or more nucleic acid sequences encoding the CAR are encoded by a single nucleic molecule in the same frame and as a single polypeptide chain. In some embodiments, the two or more CARs, can, for example, be separated by one or more peptide cleavage sites. (for example, an auto-cleavage site or a substrate for an intracellular protease). Examples of peptide cleavage sites include T2A, P2A, E2A, or F2A sites.

Methods of introducing and expressing genes into a cell are known in the art. In the context of an expression vector, the vector can be readily introduced into a host cell, for example, mammalian, bacterial, yeast, or insect cell by any method, for example, one known in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY). A suitable method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, for example, human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (for example, an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In some embodiments, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present disclosure, in order to confirm the presence of the recombinant nucleic acid sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, for example, by immunological means (ELISAs and Western blots) or by assays described herein to identify agents falling within the scope of this disclosure.

### Natural Killer Cell Receptor (NKR) CARs

In some embodiments, the CAR molecule described herein comprises one or more components of a natural killer cell receptor (NKR), thereby forming an NKR-CAR. The NKR component can be a transmembrane domain, a hinge domain, or a cytoplasmic domain from any of the following natural killer cell receptors: killer cell immunoglobulin-like receptor (KIR), for example, KIR2DL1, KIR2DL2/L3, KIR2DL4, KIR2DL5A, KIR2DL5B, KIR2DS1, KIR2DS2, KIR2DS3, KIR2DS4, DIR2DS5, KIR3DL1/S1, KIR3DL2, KIR3DL3, KIR2DP1, and KIR3DP1; natural cytotoxicity receptor (NCR), for example, NKp30, NKp44, NKp46; signaling lymphocyte activation molecule (SLAM) family of immune cell receptors, for example, CD48, CD229, 2B4, CD84, NTB-A, CRACC, BLAME, and CD2F-10; Fc receptor (FcR), for example, CD16, and CD64; and Ly49 receptors, for example, LY49A, LY49C. The NKR-CAR molecules described herein may interact with an adaptor molecule or intracellular signaling domain, for example, DAP12. Exemplary configurations and sequences of CAR molecules comprising NKR components are described in International Publication No. WO2014/145252, the contents of which are hereby incorporated by reference.

### Split CAR

In some embodiments, the CAR-expressing cell uses a split CAR. The split CAR approach is described in more detail in publications WO2014/055442 and WO2014/055657. Briefly, a split CAR system comprises a cell expressing a first CAR having a first antigen binding domain and a costimulatory domain (for example, 41BB), and the cell also expresses a second CAR having a second antigen binding domain and an intracellular signaling domain (for example, CD3 zeta). When the cell encounters the first antigen, the costimulatory domain is activated, and the cell proliferates. When the cell encounters the second antigen, the intracellular signaling domain is activated and cell-killing activity begins. Thus, the CAR-expressing cell is only fully activated in the presence of both antigens.

### RNA Transfection

Disclosed herein are methods for producing an in vitro transcribed RNA CAR. RNA CAR and methods of using the same are described, for example, in paragraphs 553-570 of in International Application WO2015/142675, filed Mar. 13, 2015, which is herein incorporated by reference in its entirety.

An immune effector cell can include a CAR encoded by a messenger RNA (mRNA). In some embodiments, the mRNA encoding a CAR described herein is introduced into an immune effector cell, for example, made by a method described herein, for production of a CAR-expressing cell.

In some embodiments, the in vitro transcribed RNA CAR can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired temple for in vitro transcription is a CAR described herein. For example, the template for the RNA CAR comprises an extracellular region comprising a single chain variable domain of an antibody to a tumor associated antigen described herein; a hinge region (for example, a hinge region described herein), a transmembrane domain (for example, a transmembrane domain described herein such as a transmembrane domain of CD8a); and a cytoplasmic region that includes an intracellular signaling domain, for example, an intracellular signaling domain described herein, for example, comprising the signaling domain of CD3-zeta and the signaling domain of 4-1BB.

In some embodiments, the DNA to be used for PCR contains an open reading frame. The DNA can be from a naturally occurring DNA sequence from the genome of an organism. In some embodiments, the nucleic acid can include some or all of the 5 and/or 3 untranslated regions (UTRs). The nucleic acid can include exons and introns. In some embodiments, the DNA to be used for PCR is a human nucleic acid sequence. In some embodiments, the DNA to be used for PCR is a human nucleic acid sequence including the 5 and 3 UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

PCR is used to generate a template for in vitro transcription of mRNA which is used for transfection. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a nucleic acid that is normally transcribed in cells (the open reading frame), including 5 and 3 UTRs. The primers can also be designed to amplify a portion of a nucleic acid that encodes a particular domain of interest. In some embodiments, the primers are designed to amplify the coding region of a human cDNA, including all or portions of the 5 and 3 UTRs. Primers useful for PCR can be generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein. The reagents and polymerase are commercially available from a number of sources.

Chemical structures with the ability to promote stability and/or translation efficiency may also be used. The RNA in embodiments has 5 and 3 UTRs. In some embodiments, the 5 UTR is between one and 3000 nucleotides in length. The length of 5 and 3 UTR sequences to be added to the coding region can be altered by different methods, including, but not limited to, designing primers for PCR that anneal to different regions of the UTRs. Using this approach, one of ordinary skill in the art can modify the 5 and 3 UTR lengths required to achieve optimal translation efficiency following transfection of the transcribed RNA.

The 5 and 3 UTRs can be the naturally occurring, endogenous 5 and 3 UTRs for the nucleic acid of interest. Alternatively, UTR sequences that are not endogenous to the nucleic acid of interest can be added by incorporating the UTR sequences into the forward and reverse primers or by any other modifications of the template. The use of UTR sequences that are not endogenous to the nucleic acid of interest can be useful for modifying the stability and/or translation efficiency of the RNA. For example, it is known that AU-rich elements in 3 UTR sequences can decrease the stability of mRNA. Therefore, 3 UTRs can be selected or designed to increase the stability of the transcribed RNA based on properties of UTRs that are well known in the art.

In some embodiments, the 5 UTR can contain the Kozak sequence of the endogenous nucleic acid. Alternatively, when a 5 UTR that is not endogenous to the nucleic acid of interest is being added by PCR as described above, a consensus Kozak sequence can be redesigned by adding the 5 UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5 UTR can be 5′UTR of an RNA virus whose RNA genome is stable in cells. In other embodiments various nucleotide analogues can be used in the 3 or 5 UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5 end of the forward primer, the RNA polymerase promoter becomes incorporated into the PCR product upstream of the open reading frame that is to be transcribed. In some embodiments, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In some embodiments, the mRNA has both a cap on the 5 end and a 3 poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3 UTR results in normal sized mRNA which is not effective in eukaryotic transfection even if it is polyadenylated after transcription.

On a linear DNA template, phage T7 RNA polymerase can extend the 3 end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of poly(A)/T stretches into a DNA template is molecular cloning. However, poly(A)/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with poly(A)/T 3 stretch without cloning highly desirable.

The poly(A)/T segment of the transcriptional DNA template can be produced during PCR by using a reverse primer containing a polyT tail, such as 100T tail (SEQ ID NO: 31) (size can be 50-5000 T (SEQ ID NO: 32)), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In some embodiments, the poly(A) tail is between 100 and 5000 adenosines (for example, SEQ ID NO: 33).

Poly(A) tails of RNAs can be further extended following in vitro transcription with the use of a poly(A) polymerase, such as E. coli poly(A) polymerase (E-PAP). In some embodiments, increasing the length of a poly(A) tail from 100 nucleotides to between 300 and 400 nucleotides (SEQ ID NO: 34) results in about a two-fold increase in the translation efficiency of the RNA. Additionally, the attachment of different chemical groups to the 3 end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5 caps on also provide stability to RNA molecules. In some embodiments, RNAs produced by the methods disclosed herein include a 5 cap. The 5 cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001).

### Non-Viral Delivery Methods

In some embodiments, non-viral methods can be used to deliver a nucleic acid encoding a CAR described herein into a cell or tissue or a subject.

In some embodiments, the non-viral method includes the use of a transposon (also called a transposable element). In some embodiments, a transposon is a piece of DNA that can insert itself at a location in a genome, for example, a piece of DNA that is capable of self-replicating and inserting its copy into a genome, or a piece of DNA that can be spliced out of a longer nucleic acid and inserted into another place in a genome. For example, a transposon comprises a DNA sequence made up of inverted repeats flanking genes for transposition.

Exemplary methods of nucleic acid delivery using a transposon include a Sleeping Beauty transposon system (SBTS) and a piggyBac™ (PB) transposon system. See, for example, Aronovich et al. Hum. Mol. Genet. 20.R1(2011):R14-20; Singh et al. Cancer Res. 15(2008):2961-2971; Huang et al. Mol. Ther. 16(2008):580-589; Grabundzija et al. Mol. Ther. 18(2010):1200-1209; Kebriaei et al. Blood. 122.21(2013):166; Williams. Molecular Therapy 16.9(2008):1515-16; Bell et al. Nat. Protoc. 2.12(2007):3153-65; and Ding et al. Cell. 122.3(2005):473-83, all of which are incorporated herein by reference.

The SBTS includes two components: 1) a transposon containing a transgene and 2) a source of transposase enzyme. The transposase can transpose the transposon from a carrier plasmid (or other donor DNA) to a target DNA, such as a host cell chromosome/genome. For example, the transposase binds to the carrier plasmid/donor DNA, cuts the transposon (including transgene(s)) out of the plasmid, and inserts it into the genome of the host cell. See, for example, Aronovich et al. supra.

Exemplary transposons include a pT2-based transposon. See, for example, Grabundzija et al. Nucleic Acids Res. 41.3(2013):1829-47; and Singh et al. Cancer Res. 68.8(2008): 2961-2971, all of which are incorporated herein by reference. Exemplary transposases include a Tc1/mariner-type transposase, for example, the SB10 transposase or the SB11 transposase (a hyperactive transposase which can be expressed, for example, from a cytomegalovirus promoter). See, for example, Aronovich et al.; Kebriaei et al.; and Grabundzija et al., all of which are incorporated herein by reference.

Use of the SBTS permits efficient integration and expression of a transgene, for example, a nucleic acid encoding a CAR described herein. Provided herein are methods of generating a cell, for example, T cell or NK cell, that stably expresses a CAR described herein, for example, using a transposon system such as SBTS.

In accordance with methods described herein, in some embodiments, one or more nucleic acids, for example, plasmids, containing the SBTS components are delivered to a cell (for example, T or NK cell). For example, the nucleic acid(s) are delivered by standard methods of nucleic acid (for example, plasmid DNA) delivery, for example, methods described herein, for example, electroporation, transfection, or lipofection. In some embodiments, the nucleic acid contains a transposon comprising a transgene, for example, a nucleic acid encoding a CAR described herein. In some embodiments, the nucleic acid contains a transposon comprising a transgene (for example, a nucleic acid encoding a CAR described herein) as well as a nucleic acid sequence encoding a transposase enzyme. In other embodiments, a system with two nucleic acids is provided, for example, a dual-plasmid system, for example, where a first plasmid contains a transposon comprising a transgene, and a second plasmid contains a nucleic acid sequence encoding a transposase enzyme. For example, the first and the second nucleic acids are co-delivered into a host cell.

In some embodiments, cells, for example, T or NK cells, are generated that express a CAR described herein by using a combination of gene insertion using the SBTS and genetic editing using a nuclease (for example, Zinc finger nucleases (ZFNs), Transcription Activator-Like Effector Nucleases (TALENs), the CRISPR/Cas system, or engineered meganuclease re-engineered homing endonucleases).

In some embodiments, use of a non-viral method of delivery permits reprogramming of cells, for 5 example, T or NK cells, and direct infusion of the cells into a subject. Advantages of non-viral vectors include but are not limited to the ease and relatively low cost of producing sufficient amounts required to meet a patient population, stability during storage, and lack of immunogenicity.

Methods of Manufacture/Production

In some embodiments, the methods disclosed herein further include administering a T cell depleting agent after treatment with the cell (for example, an immune effector cell as described herein), thereby reducing (for example, depleting) the CAR-expressing cells (for example, the CD19CAR-expressing cells). Such T cell depleting agents can be used to effectively deplete CAR-expressing cells (for example, CD19CAR-expressing cells) to mitigate toxicity. In some embodiments, the CAR-expressing cells were manufactured according to a method herein, for example, assayed (for example, before or after transfection or transduction) according to a method herein.

In some embodiments, the T cell depleting agent is administered one, two, three, four, or five weeks after administration of the cell, for example, the population of immune effector cells, described herein.

In some embodiments, the T cell depleting agent is an agent that depletes CAR-expressing cells, for example, by inducing antibody dependent cell-mediated cytotoxicity (ADCC) and/or complement-induced cell death. For example, CAR-expressing cells described herein may also express an antigen (for example, a target antigen) that is recognized by molecules capable of inducing cell death, for example, ADCC or complement-induced cell death. For example, CAR expressing cells described herein may also express a target protein (for example, a receptor) capable of being targeted by an antibody or antibody fragment. Examples of such target proteins include, but are not limited to, EpCAM, VEGFR, integrins (for example, integrins αvβ3, α4, αI3/4β3, α4β7, α5β1, αvβ3, αv), members of the TNF receptor superfamily (for example, TRAIL-R1 , TRAIL-R2), PDGF Receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CD5, CD11 , CD11a/LFA-1, CD15, CD18/ITGB2, CD19, CD20, CD22, CD23/IgE Receptor, CD25, CD28, CD30, CD33, CD38, CD40, CD41, CD44, CD51 , CD52, CD62L, CD74, CD80, CD125, CD147/basigin, CD152/CTLA-4, CD154/CD40L, CD195/CCR5, CD319/SLAMF7, and EGFR, and truncated versions thereof (for example, versions preserving one or more extracellular epitopes but lacking one or more regions within the cytoplasmic domain).

In some embodiments, the CAR expressing cell co-expresses the CAR and the target protein, for example, naturally expresses the target protein or is engineered to express the target protein. For example, the cell, for example, the population of immune effector cells, can include a nucleic acid (for example, vector) comprising the CAR nucleic acid (for example, a CAR nucleic acid as described herein) and a nucleic acid encoding the target protein.

In some embodiments, the T cell depleting agent is a CD52 inhibitor, for example, an anti-CD52 antibody molecule, for example, alemtuzumab.

In other embodiments, the cell, for example, the population of immune effector cells, expresses a CAR molecule as described herein (for example, CD19CAR) and the target protein recognized by the T cell depleting agent. In some embodiments, the target protein is CD20. In embodiments where the target protein is CD20, the T cell depleting agent is an anti-CD20 antibody, for example, rituximab.

In further embodiments of any of the aforesaid methods, the methods further include transplanting a cell, for example, a hematopoietic stem cell, or a bone marrow, into the mammal.

In some embodiments, this disclosure features a method of conditioning a mammal prior to cell transplantation. The method includes administering to the mammal an effective amount of the cell comprising a CAR nucleic acid or polypeptide, for example, a CD19 CAR nucleic acid or polypeptide. In some embodiments, the cell transplantation is a stem cell transplantation, for example, a hematopoietic stem cell transplantation, or a bone marrow transplantation. In other embodiments, conditioning a subject prior to cell transplantation includes reducing the number of target-expressing cells in a subject, for example, CD19-expressing normal cells or CD19-expressing cancer cells.

### Elutriation

In some embodiments, the methods described herein feature an elutriation method that removes unwanted cells, for example, monocytes and blasts, thereby resulting in an improved enrichment of desired immune effector cells suitable for CAR expression. In some embodiments, the elutriation method described herein is optimized for the enrichment of desired immune effector cells suitable for CAR expression from a previously frozen sample, for example, a thawed sample. In some embodiments, the elutriation method described herein provides a preparation of cells with improved purity as compared to a preparation of cells collected from the elutriation protocols known in the art. In some embodiments, the elutriation method described herein includes using an optimized viscosity of the starting sample, for example, cell sample, for example, thawed cell sample, by dilution with certain isotonic solutions (for example, PBS), and using an optimized combination of flow rates and collection volume for each fraction collected by an elutriation device. Exemplary elutriation methods that could be applied in the present disclosure are described on pages 48-51 of WO 2017/117112, herein incorporated by reference in its entirety.

### Density Gradient Centrifugation

Manufacturing of adoptive cell therapeutic product requires processing the desired cells, for example, immune effector cells, away from a complex mixture of blood cells and blood elements present in peripheral blood apheresis starting materials. Peripheral blood-derived lymphocyte samples have been successfully isolated using density gradient centrifugation through Ficoll solution. However, Ficoll is not a preferred reagent for isolating cells for therapeutic use, as Ficoll is not qualified for clinical use. In addition, Ficoll contains glycol, which has toxic potential to the cells. Furthermore, Ficoll density gradient centrifugation of thawed apheresis products after cryopreservation yields a suboptimal T cell product, for example, as described in the Examples herein. For example, a loss of T cells in the final product, with a relative gain of non-T cells, especially undesirable B cells, blast cells and monocytes was observed in cell preparations isolated by density gradient centrifugation through Ficoll solution.

Without wishing to be bound by theory, it is believed that immune effector cells, for example, T cells, dehydrate during cryopreservation to become denser than fresh cells. Without wishing to be bound by theory, it is also believed that immune effector cells, for example, T cells, remain denser longer than the other blood cells, and thus are more readily lost during Ficoll density gradient separation as compared to other cells. Accordingly, without wishing to be bound by theory, a medium with a density greater than Ficoll is believed to provide improved isolation of desired immune effector cells in comparison to Ficoll or other mediums with the same density as Ficoll, for example, 1.077 g/mL.

In some embodiments, the density gradient centrifugation method described herein includes the use of a density gradient medium comprising iodixanol. In some embodiments, the density gradient medium comprises about 60% iodixanol in water.

In some embodiments, the density gradient centrifugation method described herein includes the use of a density gradient medium having a density greater than Ficoll. In some embodiments, the density gradient centrifugation method described herein includes the use of a density gradient medium having a density greater than 1.077 g/mL, for example, greater than 1.077 g/mL, greater than 1.1 g/mL, greater than 1.15 g/mL, greater than 1.2 g/mL, greater than 1.25 g/mL, greater than 1.3 g/mL, greater than 1.31 g/mL. In some embodiments, the density gradient medium has a density of about 1.32 g/mL.

Additional embodiments of density gradient centrifugation are described on pages 51-53 of WO 2017/117112, herein incorporated by reference in its entirety.

### Enrichment by Selection

Provided herein are methods for selection of specific cells to improve the enrichment of the desired immune effector cells suitable for CAR expression. In some embodiments, the selection comprises a positive selection, for example, selection for the desired immune effector cells. In some embodiments, the selection comprises a negative selection, for example, selection for unwanted cells, for example, removal of unwanted cells. In embodiments, the positive or negative selection methods described herein are performed under flow conditions, for example, by using a flow-through device, for example, a flow-through device described herein. Exemplary positive and negative selections are described on pages 53-57 of WO 2017/117112, herein incorporated by reference in its entirety. Selection methods can be performed under flow conditions, for example, by using a flow-through device, also referred to as a cell processing system, to further enrich a preparation of cells for desired immune effector cells, for example, T cells, suitable for CAR expression. Exemplary flow-through devices are described on pages 57-70 of WO 2017/117112, herein incorporated by reference in its entirety. Exemplary cell separation and debeading methods are described on pages 70-78 of WO 2017/117112, herein incorporated by reference in its entirety.

Selection procedures are not limited to ones described on pages 57-70 of WO 2017/117112. Negative T cell selection via removal of unwanted cells with CD19, CD14 and CD26 Miltenyi beads in combination with column technology (CliniMACS® Plus or CliniMACS® Prodigy®) or positive T cell selection with a combination of CD4 and CD8 Miltenyi beads and column technology (CliniMACS® Plus or CliniMACS® Prodigy®) can be used. Alternatively, column-free technology with releasable CD3 beads (GE Healthcare) can be used.

In addition, bead-free technologies such as ThermoGenesis X-series devices can be utilized as well.

### Clinical Applications

All of the processes herein may be conducted according to clinical good manufacturing practice (cGMP) standards.

The processes may be used for cell purification, enrichment, harvesting, washing, concentration or for cell media exchange, particularly during the collection of raw, starting materials (particularly cells) at the start of the manufacturing process, as well as during the manufacturing process for the selection or expansion of cells for cell therapy.

The cells may include any plurality of cells. The cells may be of the same cell type, or mixed cell types. In addition, the cells may be from one donor, such as an autologous donor or a single allogenic donor for cell therapy. The cells may be obtained from patients by, for example, leukapheresis or apheresis. The cells may include T cells, for example may include a population that has greater than 50% T cells, greater than 60% T cells, greater than 70% T cells, greater than 80% T cells, or 90% T cells.

Selection processes may be particularly useful in selecting cells prior to culture and expansion. For instance, paramagnetic particles coated with anti-CD3 and/or anti CD28 may be used to select T cells for expansion or for introduction of a nucleic acid encoding a chimeric antigen receptor (CAR) or other protein. Such a process is used to produce CTL019 T cells for treatment of acute lymphoblastic leukemia (ALL).

The debeading processes and modules disclosed herein may be particularly useful in the manufacture of cells for cell therapy, for example in purifying cells prior to, or after, culture and expansion. For instance, paramagnetic particles coated with anti-CD3 and/or anti CD28 antibodies may be used to selectively expand T cells, for example T cells that are, or will be, modified by introduction of a nucleic acid encoding a chimeric antigen receptor (CAR) or other protein, such that the CAR is expressed by the T cells. During the manufacture of such T cells, the debeading processes or modules may be used to separate T cells from the paramagnetic particles. Such a debeading process or module is used to produce, for example, CTL019 T cells for treatment of acute lymphoblastic leukemia (ALL).

In one such process, illustrated here by way of example, cells, for example, T cells, are collected from a donor (for example, a patient to be treated with an autologous chimeric antigen receptor T cell product) via apheresis (for example, leukapheresis). Collected cells may then be optionally purified, for example, by an elutriation step, or via positive or negative selection of target cells (for example, T cells). Paramagnetic particles, for example, anti-CD3/anti-CD28-coated paramagnetic particles, may then be added to the cell population, to expand the T cells. The process may also include a transduction step, wherein nucleic acid encoding one or more desired proteins, for example, a CAR, for example a CAR targeting CD19, is introduced into the cell. The nucleic acid may be introduced in a lentiviral vector. The cells, for example, the lentivirally transduced cells, may then be expanded for a period of days, for example, 1, 2, 3, 4, 5, 6, 7, 8, 9, 10, or more days, for example in the presence of a suitable medium. After expansion, the debeading processes/modules disclosed herein may be used to separate the desired T cells from the paramagnetic particles. The process may include one or more debeading steps according to the processes of the present disclosure. The debeaded cells may then be formulated for administration to the patient. Examples of CAR T cells and their manufacture are further described, for example, in WO2012/079000, which is incorporated herein by reference in its entirety. The systems and methods of the present disclosure may be used for any cell separation/purification/debeading processes described in or associated with WO2012/079000. Additional CART manufacturing processes are described in, for example, WO2016109410 and WO2017117112, herein incorporated by reference in their entireties.

The systems and methods herein may similarly benefit other cell therapy products by wasting fewer desirable cells, causing less cell trauma, and more reliably removing magnetic and any non-paramagnetic particles from cells with less or no exposure to chemical agents, as compared to conventional systems and methods.

Although only exemplary embodiments of this disclosure are specifically described above, it will be appreciated that modifications and variations of these examples are possible without departing from the spirit and intended scope of this disclosure. For example, the magnetic modules and systems containing them may be arranged and used in a variety of configurations in addition to those described. Besides, non-magnetic modules can be utilized as well. In addition, the systems and methods may include additional components and steps not specifically described herein. For instance, methods may include priming, where a fluid is first introduced into a component to remove bubbles and reduce resistance to cell suspension or buffer movement. Furthermore, embodiments may include only a portion of the systems described herein for use with the methods described herein. For example, embodiments may relate to disposable modules, hoses, etc. usable within non-disposable equipment to form a complete system able to separate or debead cells to produce a cell product.

Additional manufacturing methods and processes that can be combined with the present disclosure have been described in the art. For examples, pages 86-91 of WO 2017/117112 describe improved wash steps and improved manufacturing process.

### Sources of Immune Effector Cells

This section provides additional methods or steps for obtaining an input sample comprising desired immune effector cells, isolating and processing desired immune effector cells, for example, T cells, and removing unwanted materials, for example, unwanted cells. The additional methods or steps described in this section can be used in combination with any of the elutriation, density gradient centrifugation, selection under flow conditions, or improved wash step described in the preceding sections.

A source of cells, for example, T cells or natural killer (NK) cells, can be obtained from a subject. Examples of subjects include humans, monkeys, chimpanzees, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors.

In some embodiments of the present disclosure, immune effector cells, for example, T cells, can be obtained from a unit of blood collected from a subject using any number of techniques known to the skilled artisan, and any of the methods disclosed herein, in any combination of steps thereof. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and, optionally, to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. In some embodiments, the cells are washed using the improved wash step described herein.

Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate™, or the Haemonetics Cell Saver 5), Haemonetics Cell Saver Elite (GE Healthcare Sepax or Sefia), or a device utilizing the spinning membrane filtration technology (Fresenius Kabi LOVO), according to the manufacturer's instructions. After washing, the cells may be resuspended in a variety of biocompatible buffers, such as, for example, Ca-free, Mg-free PBS, PlasmaLyte A, PBS-EDTA supplemented with human serum albumin (HSA), or other saline solution with or without buffer. Alternatively, the undesirable components of the apheresis sample may be removed and the cells directly resuspended in culture media.

In some embodiments, desired immune effector cells, for example, T cells, are isolated from peripheral blood lymphocytes by lysing the red blood cells and depleting the monocytes, for example, by centrifugation through a PERCOLL™ gradient or by counterflow centrifugal elutriation.

The methods described herein can include, for example, selection of a specific subpopulation of immune effector cells, for example, T cells, that are a T regulatory cell-depleted population, for example, CD25+ depleted cells or CD25^{high} depleted cells, using, for example, a negative selection technique, for example, described herein. In some embodiments, the population of T regulatory-depleted cells contains less than 30%, 25%, 20%, 15%, 10%, 5%, 4%, 3%, 2%, 1% of CD25+ cells or CD25^{high} cells.

In some embodiments, T regulatory cells, for example, CD25+ T cells or CD25^{high} T cells, are removed from the population using an anti-CD25 antibody, or fragment thereof, or a CD25-binding ligand, for example IL-2. In some embodiments, the anti-CD25 antibody, or fragment thereof, or CD25-binding ligand is conjugated to a substrate, for example, a bead, or is otherwise coated on a substrate, for example, a bead. In some embodiments, the anti-CD25 antibody, or fragment thereof, is conjugated to a substrate as described herein.

In some embodiments, the T regulatory cells, for example, CD25+ T cells or CD25^{high} T cells, are removed from the population using CD25 depleting reagent from Miltenyi™. In some embodiments, the ratio of cells to CD25 depletion reagent is 1e7 cells to 20 μL, or 1e7 cells to 15 μL, or 1e7 cells to 10 μL, or 1e7 cells to 5 μL, or 1e7 cells to 2.5 μL, or 1e7 cells to 1.25 μL. In some embodiments, for example, for T regulatory cells, greater than 500 million cells/ml is used. In some embodiments, a concentration of cells of 600, 700, 800, or 900 million cells/ml is used.

In some embodiments, the population of immune effector cells to be depleted includes about 6×10⁹ CD25+ T cells. In some embodiments, the population of immune effector cells to be depleted include about 1×10⁹ to 1×10¹⁰ CD25+ T cell, and any integer value in between. In some embodiments, the resulting population T regulatory-depleted cells has 2×10⁹T regulatory cells, for example, CD25+ cells or CD25^{high} cells, or less (for example, 1×10⁹, 5×10⁸, 1×10⁸, 5×10⁷, 1×10⁷, or less T regulatory cells).

In some embodiments, the T regulatory cells, for example, CD25+ cells or CD25^{high} cells, are removed from the population using the CliniMAC system with a depletion tubing set, such as, for example, tubing 162-01. In some embodiments, the CliniMAC system is run on a depletion setting such as, for example, DEPLETION2.1.

Without wishing to be bound by a particular theory, decreasing the level of negative regulators of immune cells (for example, decreasing the number of unwanted immune cells, for example, Treg cells), in a subject prior to apheresis or during manufacturing of a CAR-expressing cell product significantly reduces the risk of subject relapse. For example, methods of depleting Treg cells are known in the art. Methods of decreasing Treg cells include, but are not limited to, cyclophosphamide, anti-GITR antibody (an anti-GITR antibody described herein), CD25-depletion, and combinations thereof.

In some embodiments, the manufacturing methods comprise reducing the number of (for example, depleting) Treg cells prior to manufacturing of the CAR-expressing cell. For example, manufacturing methods comprise contacting the sample, for example, the apheresis sample, with an anti-GITR antibody and/or an anti-CD25 antibody (or fragment thereof, or a CD25-binding ligand), for example, to deplete Treg cells prior to manufacturing of the CAR-expressing cell (for example, T cell, NK cell) product.

Without wishing to be bound by a particular theory, decreasing the level of negative regulators of immune cells (for example, decreasing the number of unwanted immune cells, for example, Treg cells), in a subject prior to apheresis or during manufacturing of a CAR-expressing cell product can reduce the risk of a subject's relapse. In some embodiments, a subject is pre-treated with one or more therapies that reduce Treg cells prior to collection of cells for CAR-expressing cell product manufacturing, thereby reducing the risk of subject relapse to CAR-expressing cell treatment. In some embodiments, methods of decreasing Treg cells include, but are not limited to, administration to the subject of one or more of cyclophosphamide, anti-GITR antibody, CD25-depletion, or a combination thereof. In some embodiments, methods of decreasing Treg cells include, but are not limited to, administration to the subject of one or more of cyclophosphamide, anti-GITR antibody, CD25-depletion, or a combination thereof. Administration of one or more of cyclophosphamide, anti-GITR antibody, CD25-depletion, or a combination thereof, can occur before, during or after an infusion of the CAR-expressing cell product. Administration of one or more of cyclophosphamide, anti-GITR antibody, CD25-depletion, or a combination thereof, can occur before, during or after an infusion of the CAR-expressing cell product.

In some embodiments, the manufacturing methods comprise reducing the number of (for example, depleting) Treg cells prior to manufacturing of the CAR-expressing cell. For example, manufacturing methods comprise contacting the sample, for example, the apheresis sample, with an anti-GITR antibody and/or an anti-CD25 antibody (or fragment thereof, or a CD25-binding ligand), for example, to deplete Treg cells prior to manufacturing of the CAR-expressing cell (for example, T cell, NK cell) product.

In some embodiments, a subject is pre-treated with cyclophosphamide prior to collection of cells for CAR-expressing cell product manufacturing, thereby reducing the risk of subject relapse to CAR-expressing cell treatment (for example, CTL019 treatment). In some embodiments, a subject is pre-treated with an anti-GITR antibody prior to collection of cells for CAR-expressing cell (for example, T cell or NK cell) product manufacturing, thereby reducing the risk of subject relapse to CAR-expressing cell treatment.

In some embodiments, the CAR-expressing cell (for example, T cell, NK cell) manufacturing process is modified to deplete Treg cells prior to manufacturing of the CAR-expressing cell (for example, T cell, NK cell) product (for example, a CTL019 product). In some embodiments, CD25-depletion is used to deplete Treg cells prior to manufacturing of the CAR-expressing cell (for example, T cell, NK cell) product (for example, a CTL019 product).

In some embodiments, the population of cells to be removed are neither the regulatory T cells or tumor cells, but cells that otherwise negatively affect the expansion and/or function of CART cells, for example cells expressing CD14, CD11b, CD33, CD15, or other markers expressed by potentially immune suppressive cells. In some embodiments, such cells are envisioned to be removed concurrently with regulatory T cells and/or tumor cells, or following said depletion, or in another order.

The methods described herein can include more than one selection step, for example, more than one depletion step. Enrichment of a T cell population by negative selection can be accomplished, for example, with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail can include antibodies to CD14, CD20, CD11b, CD16, HLA-DR, and CD8.

The methods described herein can further include removing cells from the population which express a tumor antigen, for example, a tumor antigen that does not comprise CD25, for example, CD19, CD30, CD38, CD123, CD20, CD14 or CD11b, to thereby provide a population of T regulatory-depleted, for example, CD25+ depleted or CD25^{high} depleted, and tumor antigen depleted cells that are suitable for expression of a CAR, for example, a CAR described herein. In some embodiments, tumor antigen expressing cells are removed simultaneously with the T regulatory, for example, CD25+ cells or CD25^{high} cells. For example, an anti-CD25 antibody, or fragment thereof, and an anti-tumor antigen antibody, or fragment thereof, can be attached to the same substrate, for example, bead, which can be used to remove the cells or an anti-CD25 antibody, or fragment thereof, or the anti-tumor antigen antibody, or fragment thereof, can be attached to separate beads, a mixture of which can be used to remove the cells. In other embodiments, the removal of T regulatory cells, for example, CD25+ cells or CD25^{high} cells, and the removal of the tumor antigen expressing cells is sequential, and can occur, for example, in either order.

Also provided are methods that include removing cells from the population which express a check point inhibitor, for example, a check point inhibitor described herein, for example, one or more of PD1+ cells, LAG3+ cells, and TIM3+ cells, to thereby provide a population of T regulatory-depleted, for example, CD25+ depleted cells, and check point inhibitor depleted cells, for example, PD1+, LAG3+ and/or TIM3+ depleted cells. Exemplary check point inhibitors include PD1, PD-L1, PD-L2, CTLA4, TIM3, CEACAM (for example, CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF (for example, TGF beta), for example, as described herein. In some embodiments, check point inhibitor expressing cells are removed simultaneously with the T regulatory, for example, CD25+ cells or CD25^{high} cells. For example, an anti-CD25 antibody, or fragment thereof, and an anti-check point inhibitor antibody, or fragment thereof, can be attached to the same bead which can be used to remove the cells, or an anti-CD25 antibody, or fragment thereof, and the anti-check point inhibitor antibody, or fragment there, can be attached to separate beads, a mixture of which can be used to remove the cells.

In other embodiments, the removal of T regulatory cells, for example, CD25+ cells or CD25^{high} cells, and the removal of the check point inhibitor expressing cells is sequential, and can occur, for example, in either order.

Methods described herein can include a positive selection step. For example, T cells can isolated by incubation with anti-CD3/anti-CD28 (for example, 3×28)-conjugated beads, such as Dynabeads® M-450 CD3/CD28 T, for a time period sufficient for positive selection of the desired T cells. In some embodiments, the time period is about 30 minutes. In some embodiments, the time period ranges from 30 minutes to 36 hours or longer and all integer values there between. In some embodiments, the time period is at least 1, 2, 3, 4, 5, or 6 hours. In some embodiments, the time period is 10 to 24 hours, for example, 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points.

In some embodiments, a T cell population can be selected that expresses one or more of IFN-γ, TNFα, IL-17A, IL-2, IL-3, IL-4, GM-CSF, IL-10, IL-13, granzyme B, and perform, or other appropriate molecules, for example, other cytokines. Methods for screening for cell expression can be determined, for example, by the methods described in PCT Publication No.: WO 2013/126712.

For isolation of a desired population of cells by positive or negative selection, the concentration of cells and surface (for example, particles such as beads) can be varied. In some embodiments, it may be desirable to significantly decrease the volume in which beads and cells are mixed together (for example, increase the concentration of cells), to ensure maximum contact of cells and beads. For example, in some embodiments, a concentration of 10 billion cells/ml, 9 billion/ml, 8 billion/ml, 7 billion/ml, 6 billion/ml, or 5 billion/ml is used. In some embodiments, a concentration of 1 billion cells/ml is used. In some embodiments, a concentration of cells from 75, 80, 85, 90, 95, or 100 million cells/ml is used. In some embodiments, concentrations of 125 or 150 million cells/ml can be used.

Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (for example, leukemic blood, tumor tissue, etc.). Such populations of cells may have therapeutic value and would be desirable to obtain. For example, using high concentration of cells allows more efficient selection of CD8+ T cells that normally have weaker CD28 expression.

In some embodiments, it may be desirable to use lower concentrations of cells. By significantly diluting the mixture of T cells and surface (for example, particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In some embodiments, the concentration of cells used is 5×10⁶/ml. In some embodiments, the concentration used can be from about 1×10⁵/ml to 1×10⁶/ml, and any integer value in between.

In some embodiments, the cells may be incubated on a rotator for varying lengths of time at varying speeds at either 2-10° C. or at room temperature.

In some embodiments, a plurality of the immune effector cells of the population do not express diaglycerol kinase (DGK), for example, is DGK-deficient. In some embodiments, a plurality of the immune effector cells of the population do not express Ikaros, for example, is Ikaros-deficient. In some embodiments, a plurality of the immune effector cells of the population do not express DGK and Ikaros, for example, is both DGK and Ikaros-deficient.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In some embodiments, cryopreserved cells are thawed and washed as described herein and allowed to rest for one hour at room temperature prior to activation using the methods of the present disclosure.

Also contemplated in the context of this disclosure is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in immune effector cell therapy for any number of diseases or conditions that would benefit from immune effector cell therapy, such as those described herein. In some embodiments a blood sample or an apheresis is taken from a generally healthy subject. In some embodiments, a blood sample or an apheresis is taken from a generally healthy subject who is at risk of developing a disease, but who has not yet developed a disease, and the cells of interest are isolated and frozen for later use. In some embodiments, the T cells may be expanded, frozen, and used at a later time. In some embodiments, samples are collected from a patient shortly after diagnosis of a particular disease as described herein but prior to any treatments. In some embodiments, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In some embodiments of the present disclosure, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present disclosure to collect blood cells, including T cells, dendritic cells, or other cells of the hematopoietic lineage, during this recovery phase. Further, in some embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

In some embodiments, the immune effector cells expressing a CAR molecule, for example, a CAR molecule described herein, are obtained from a subject that has received a low, immune enhancing dose of an mTOR inhibitor. In some embodiments, the population of immune effector cells, for example, T cells, to be engineered to express a CAR, are harvested after a sufficient time, or after sufficient dosing of the low, immune enhancing, dose of an mTOR inhibitor, such that the level of PD1 negative immune effector cells, for example, T cells, or the ratio of PD1 negative immune effector cells, for example, T cells/PD1 positive immune effector cells, for example, T cells, in the subject or harvested from the subject has been, at least transiently, increased.

In other embodiments, population of immune effector cells, for example, T cells, which have, or will be engineered to express a CAR, can be treated ex vivo by contact with an amount of an mTOR inhibitor that increases the number of PD1 negative immune effector cells, for example, T cells or increases the ratio of PD1 negative immune effector cells, for example, T cells/PD1 positive immune effector cells, for example, T cells.

It is recognized that the methods of the application can utilize culture media conditions comprising 5% or less, for example 2%, human AB serum, and employ known culture media conditions and compositions, for example those described in Smith et al., “Ex vivo expansion of human T cells for adoptive immunotherapy using the novel Xeno-free CTS™ Immune Cell Serum Replacement” Clinical & Translational Immunology (2015) 4, e31; doi:10.1038/cti.2014.31.

In some embodiments, the methods of the application can utilize media conditions comprising at least about 0.1%, 0.5%, 1.0%, 1.5%, 2%, 2.5%, 3%, 3.5%, 4%, 4.5%, 5%, 6%, 7%, 8%, 9% or 10% serum. In some embodiments, the media comprises about 0.5%-5%, about 0.5%-4.5%, about 0.5%-4%, about 0.5%-3.5%, about 0.5%-3%, about 0.5%-2.5%, about 0.5%-2%, about 0.5%-1.5%, about 0.5%-1.0%, about 1.0%-5%, about 1.5%-5%, about 2%-5%, about 2.5%-5%, about 3%-5%, about 3.5%-5%, about 4%-5%, or about 4.5%-5% serum. In some embodiments, the media comprises about 0.5% serum.

In some embodiments, the media comprises about 0.5% serum. In some embodiments, the media comprises about 1% serum. In some embodiments, the media comprises about 1.5% serum. In some embodiments, the media comprises about 2% serum. In some embodiments, the media comprises about 2.5% serum. In some embodiments, the media comprises about 3% serum. In some embodiments, the media comprises about 3.5% serum. In some embodiments, the media comprises about 4% serum. In some embodiments, the media comprises about 4.5% serum. In some embodiments, the media comprises about 5% serum. In some embodiments, the serum comprises human serum, e.g., human AB serum. In some embodiments, the serum is human serum that has been allowed to naturally coagulate after collection, e.g., off-the-clot (OTC) serum. In some embodiments, the serum is plasma-derived serum human serum. Plasma-derived serum can be produced by defibrinating pooled human plasma collected in the presence of an anticoagulant, e.g., sodium citrate.

In some embodiments, the methods of the application can utilize culture media conditions comprising serum-free medium. In some embodiments, the serum free medium is OpTmizer™ CTS™ (LifeTech), Immunocult™ XF (Stemcell technologies), CellGro™ (CellGenix), TexMacs™ (Miltenyi), Stemline™ (Sigma), Xvivol5™ (Lonza), PrimeXV® (Irvine Scientific), or StemXVivo® (RandD systems). The serum-free medium can be supplemented with a serum substitute such as ICSR (immune cell serum replacement) from LifeTech. The level of serum substitute (for example, ICSR) can be, for example, up to 5%, for example, about 1%, 2%, 3%, 4%, or 5%. In some embodiments, the serum-free medium can be supplemented with serum, e.g., human serum, e.g., human AB serum. In some embodiments, the serum is human serum that has been allowed to naturally coagulate after collection, e.g., off-the-clot (OTC) serum. In some embodiments, the serum is plasma-derived human serum. Plasma-derived serum can be produced by defibrinating pooled human plasma collected in the presence of an anticoagulant, e.g., sodium citrate.

In some embodiments, a T cell population is diaglycerol kinase (DGK)-deficient. DGK-deficient cells include cells that do not express DGK RNA or protein, or have reduced or inhibited DGK activity. DGK-deficient cells can be generated by genetic approaches, for example, administering RNA-interfering agents, for example, siRNA, shRNA, miRNA, to reduce or prevent DGK expression. Alternatively, DGK-deficient cells can be generated by treatment with DGK inhibitors described herein.

In some embodiments, a T cell population is Ikaros-deficient. Ikaros-deficient cells include cells that do not express Ikaros RNA or protein, or have reduced or inhibited Ikaros activity, Ikaros-deficient cells can be generated by genetic approaches, for example, administering RNA-interfering agents, for example, siRNA, shRNA, miRNA, to reduce or prevent Ikaros expression. Alternatively, Ikaros-deficient cells can be generated by treatment with Ikaros inhibitors, for example, lenalidomide.

In embodiments, a T cell population is DGK-deficient and Ikaros-deficient, for example, does not express DGK and Ikaros, or has reduced or inhibited DGK and Ikaros activity. Such DGK and Ikaros-deficient cells can be generated by any of the methods described herein.

In some embodiments, the NK cells are obtained from the subject. In some embodiments, the NK cells are an NK cell line, for example, NK-92 cell line (Conkwest).

### Allogeneic CAR-Expressing Cells

In embodiments described herein, the immune effector cell can be an allogeneic immune effector cell, for example, T cell or NK cell. For example, the cell can be an allogeneic T cell, for example, an allogeneic T cell lacking expression of a functional T cell receptor (TCR) and/or human leukocyte antigen (HLA), for example, HLA class I and/or HLA class II.

A T cell lacking a functional TCR can be, for example, engineered such that it does not express any functional TCR on its surface, engineered such that it does not express one or more subunits that comprise a functional TCR (for example, engineered such that it does not express (or exhibits reduced expression) of TCR alpha, TCR beta, TCR gamma, TCR delta, TCR epsilon, and/or TCR zeta) or engineered such that it produces very little functional TCR on its surface. Alternatively, the T cell can express a substantially impaired TCR, for example, by expression of mutated or truncated forms of one or more of the subunits of the TCR. The term “substantially impaired TCR” means that this TCR will not elicit an adverse immune reaction in a host.

A T cell described herein can be, for example, engineered such that it does not express a functional HLA on its surface. For example, a T cell described herein, can be engineered such that cell surface expression HLA, for example, HLA class 1 and/or HLA class II, is downregulated. In some embodiments, downregulation of HLA may be accomplished by reducing or eliminating expression of beta-2 microglobulin (B2M).

In some embodiments, the T cell can lack a functional TCR and a functional HLA, for example, HLA class I and/or HLA class II.

Modified T cells that lack expression of a functional TCR and/or HLA can be obtained by any suitable means, including a knock out or knock down of one or more subunit of TCR or HLA. For example, the T cell can include a knock down of TCR and/or HLA using siRNA, shRNA, clustered regularly interspaced short palindromic repeats (CRISPR) transcription-activator like effector nuclease (TALEN), or zinc finger endonuclease (ZFN).

In some embodiments, the allogeneic cell can be a cell which does not express or expresses at low levels an inhibitory molecule, for example by any method described herein. For example, the cell can be a cell that does not express or expresses at low levels an inhibitory molecule, for example, that can decrease the ability of a CAR-expressing cell to mount an immune effector response. Examples of inhibitory molecules include PD1, PD-L1, PD-L2, CTLA4, TIM3, CEACAM (for example, CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF (for example, TGF beta). Inhibition of an inhibitory molecule, for example, by inhibition at the DNA, RNA or protein level, can optimize a CAR-expressing cell performance. In embodiments, an inhibitory nucleic acid, for example, an inhibitory nucleic acid, for example, a dsRNA, for example, an siRNA or shRNA, a clustered regularly interspaced short palindromic repeats (CRISPR), a transcription-activator like effector nuclease (TALEN), or a zinc finger endonuclease (ZFN), for example, as described herein, can be used.

siRNA and shRNA to Inhibit TCR or HLA

In some embodiments, TCR expression and/or HLA expression can be inhibited using siRNA or shRNA that targets a nucleic acid encoding a TCR and/or HLA , and/or an inhibitory molecule described herein (for example, PD1, PD-L1, PD-L2, CTLA4, TIM3, CEACAM (for example, CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, for example, T cell.

Expression systems for siRNA and shRNAs, and exemplary shRNAs, are described, for example, in paragraphs 649 and 650 of International Application WO2015/142675, filed Mar. 13, 2015, which is incorporated by reference in its entirety.

### CRISPR to Inhibit TCR or HLA

“CRISPR” or “CRISPR to TCR and/or HLA” or “CRISPR to inhibit TCR and/or HLA” as used herein refers to a set of clustered regularly interspaced short palindromic repeats, or a system comprising such a set of repeats. “Cas”, as used herein, refers to a CRISPR-associated protein. A “CRISPR/Cas” system refers to a system derived from CRISPR and Cas which can be used to silence or mutate a TCR and/or HLA gene, and/or an inhibitory molecule described herein (for example, PD1, PD-L1, PD-L2, CTLA4, TIM3, CEACAM (for example, CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, for example, T cell.

The CRISPR/Cas system, and uses thereof, are described, for example, in paragraphs 651-658 of International Application WO2015/142675, filed March 13, 2015, which is incorporated by reference in its entirety.

TALEN to Inhibit TCR and/or HLA

“TALEN” or “TALEN to HLA and/or TCR” or “TALEN to inhibit HLA and/or TCR” refers to a transcription activator-like effector nuclease, an artificial nuclease which can be used to edit the HLA and/or TCR gene, and/or an inhibitory molecule described herein (for example, PD1, PD-L1, PD-L2, CTLA4, TIM3, CEACAM (for example, CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, for example, T cell.

TALENs , and uses thereof, are described, for example, in paragraphs 659-665 of International Application WO2015/142675, filed Mar. 13, 2015, which is incorporated by reference in its entirety.

Zinc Finger Nuclease to Inhibit HLA and/or TCR

“ZFN” or “Zinc Finger Nuclease” or “ZFN to HLA and/or TCR” or “ZFN to inhibit HLA and/or TCR” refer to a zinc finger nuclease, an artificial nuclease which can be used to edit the HLA and/or TCR gene, and/or an inhibitory molecule described herein (for example, PD1, PD-L1, PD-L2, CTLA4, TIM3, CEACAM (for example, CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF beta), in a cell, for example, T cell.

ZFNs, and uses thereof, are described, for example, in paragraphs 666-671 of International Application WO2015/142675, filed Mar. 13, 2015, which is incorporated by reference in its entirety.

### Telomerase Expression

Telomeres play a crucial role in somatic cell persistence, and their length is maintained by telomerase (TERT). Telomere length in CLL cells may be very short (Roth et al., “Significantly shorter telomeres in T-cells of patients with ZAP-70+/CD38 chronic lymphocytic leukaemia” British Journal of Haematology, 143, 383-386., Aug. 28, 2008), and may be even shorter in manufactured CAR-expressing cells, for example, CART19 cells, limiting their potential to expand after adoptive transfer to a patient. Telomerase expression can rescue CAR-expressing cells from replicative exhaustion.

While not wishing to be bound by any particular theory, in some embodiments, a therapeutic T cell has short term persistence in a patient, due to shortened telomeres in the T cell; accordingly, transfection with a telomerase gene can lengthen the telomeres of the T cell and improve persistence of the T cell in the patient. See Carl June, “Adoptive T cell therapy for cancer in the clinic”, Journal of Clinical Investigation, 117:1466-1476 (2007). Thus, in some embodiments, an immune effector cell, for example, a T cell, ectopically expresses a telomerase subunit, for example, the catalytic subunit of telomerase, for example, TERT, for example, hTERT. In some embodiments, this disclosure provides a method of producing a CAR-expressing cell, comprising contacting a cell with a nucleic acid encoding a telomerase subunit, for example, the catalytic subunit of telomerase, for example, TERT, for example, hTERT. The cell may be contacted with the nucleic acid before, simultaneous with, or after being contacted with a construct encoding a CAR.

Telomerase expression may be stable (for example, the nucleic acid may integrate into the cell's genome) or transient (for example, the nucleic acid does not integrate, and expression declines after a period of time, for example, several days). Stable expression may be accomplished by transfecting or transducing the cell with DNA encoding the telomerase subunit and a selectable marker, and selecting for stable integrants. Alternatively or in combination, stable expression may be accomplished by site-specific recombination, for example, using the Cre/Lox or FLP/FRT system.

Transient expression may involve transfection or transduction with a nucleic acid, for example, DNA or RNA such as mRNA. In some embodiments, transient mRNA transfection avoids the genetic instability sometimes associated with stable transfection with TERT. Transient expression of exogenous telomerase activity is described, for example, in International Application WO2014/130909, which is incorporated by reference herein in its entirety. In embodiments, mRNA-based transfection of a telomerase subunit is performed according to the messenger RNA Therapeutics™ platform commercialized by Moderna Therapeutics. For instance, the method may be a method described in U.S. Pat. No. 8,710,200, 8,822,663, 8,680,069, 8,754,062, 8,664,194, or 8,680,069.

In some embodiments, hTERT has the amino acid sequence of GenBank Protein ID AAC51724.1 (Meyerson et al., “hEST2, the Putative Human Telomerase Catalytic Subunit Gene, Is Up-Regulated in Tumor Cells and during Immortalization” Cell Volume 90, Issue 4, 22 Aug. 1997, Pages 785-795):

(SEQ ID NO: 284)
MPRAPRCRAVRSLLRSHYREVLPLATFVRRLGPQGWRLVQRGDPAAFRA
LVAQCLVCVPWDARPPPAAPSFRQVSCLKELVARVLQRLCERGAKNVLA
FGFALLDGARGGPPEAFTTSVRSYLPNTVTDALRGSGAWGLLLRRVGDD
VLVHLLARCALFVLVAPSCAYQVCGPPLYQLGAATQARPPPHASGPRRR
LGCERAWNHSVREAGVPLGLPAPGARRRGGSASRSLPLPKRPRRGAAPE
PERTPVGQGSWAHPGRTRGPSDRGFCVVSPARPAEEATSLEGALSGTRH
SHPSVGRQHHAGPPSTSRPPRPWDTPCPPVYAETKHFLYSSGDKEQLRP
SFLLSSLRPSLTGARRLVETIFLGSRPWMPGTPRRLPRLPQRYWQMRPL
FLELLGNHAQCPYGVLLKTHCPLRAAVTPAAGVCAREKPQGSVAAPEEE
DTDPRRLVQLLRQHSSPWQVYGFVRACLRRLVPPGLWGSRHNERRFLRN
TKKFISLGKHAKLSLQELTWKMSVRGCAWLRRSPGVGCVPAAEHRLREE
ILAKFLHWLMSVYVVELLRSFFYVTETTFQKNRLFFYRKSVWSKLQSIG
IRQHLKRVQLRELSEAEVRQHREARPALLTSRLRFIPKPDGLRPIVNMD
YVVGARTFRREKRAERLTSRVKALFSVLNYERARRPGLLGASVLGLDDI
HRAWRTFVLRVRAQDPPPELYFVKVDVTGAYDTIPQDRLTEVIASIIKP
QNTYCVRRYAVVQKAAHGHVRKAFKSHVSTLTDLQPYMRQFVAHLQETS
PLRDAVVIEQSSSLNEASSGLFDVFLRFMCHHAVRIRGKSYVQCQGIPQ
GSILSTLLCSLCYGDMENKLFAGIRRDGLLLRLVDDFLLVTPHLTHAKT
FLRTLVRGVPEYGCVVNLRKTVVNFPVEDEALGGTAFVQMPAHGLFPWC
GLLLDTRTLEVQSDYSSYARTSIRASLTFNRGFKAGRNMRRKLFGVLRL
KCHSLFLDLQVNSLQTVCTNIYKILLLQAYRFHACVLQLPFHQQVWKNP
TFFLRVISDTASLCYSILKAKNAGMSLGAKGAAGPLPSEAVQWLCHQAF
LLKLTRHRVTYVPLLGSLRTAQTQLSRKLPGTTLTALEAAANPALPSDF
KTILD

In some embodiments, the hTERT has a sequence at least 80%, 85%, 90%, 95%, 96%, 97%, 98%, or 99% identical to the sequence of SEQ ID NO: 284. In some embodiments, the hTERT has a sequence of SEQ ID NO: 284. In some embodiments, the hTERT comprises a deletion (for example, of no more than 5, 10, 15, 20, or 30 amino acids) at the N-terminus, the C-terminus, or both. In some embodiments, the hTERT comprises a transgenic amino acid sequence (for example, of no more than 5, 10, 15, 20, or 30 amino acids) at the N-terminus, the C-terminus, or both.

In some embodiments, the hTERT is encoded by the nucleic acid sequence of GenBank Accession No. AF018167 (Meyerson et al., “hEST2, the Putative Human Telomerase Catalytic Subunit Gene, Is Up-Regulated in Tumor Cells and during Immortalization” Cell Volume 90, Issue 4, 22 Aug. 1997, Pages 785-795).

### Activation and Expansion of Immune Effector Cells (For Example, T Cells)

Immune effector cells such as T cells generated or enriched by the methods described herein may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, a population of immune effector cells may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (for example, bryostatin) in conjunction with a calcium ionophore. For costimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In some embodiments, the primary stimulatory signal and the costimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In some embodiments, the agent providing the costimulatory signal is bound to a cell surface and the agent providing the primary activation signal is in solution or coupled to a surface. In some embodiments, both agents can be in solution. In some embodiments, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present disclosure.

In some embodiments, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the costimulatory signal is an anti-CD28 antibody or antigen-binding fragment thereof; and both agents are co-immobilized to the same bead in equivalent molecular amounts. In some embodiments, a 1:1 ratio of each antibody bound to the beads for CD4+ T cell expansion and T cell growth is used. In some embodiments of the present disclosure, a ratio of anti CD3:CD28 antibodies bound to the beads is used such that an increase in T cell expansion is observed as compared to the expansion observed using a ratio of 1:1. In some embodiments an increase of from about 1 to about 3 fold is observed as compared to the expansion observed using a ratio of 1:1. In some embodiments, the ratio of CD3:CD28 antibody bound to the beads ranges from 100:1 to 1:100 and all integer values there between. In some embodiments, more anti-CD28 antibody is bound to the particles than anti-CD3 antibody, i.e., the ratio of CD3:CD28 is less than one. In some embodiments, the ratio of anti CD28 antibody to anti CD3 antibody bound to the beads is greater than 2:1. In some embodiments, a 1:100 CD3:CD28 ratio of antibody bound to beads is used. In some embodiments, a 1:75 CD3:CD28 ratio of antibody bound to beads is used. In some embodiments, a 1:50 CD3:CD28 ratio of antibody bound to beads is used. In some embodiments, a 1:30 CD3:CD28 ratio of antibody bound to beads is used. In some embodiments, a 1:10 CD3:CD28 ratio of antibody bound to beads is used. In some embodiments, a 1:3 CD3:CD28 ratio of antibody bound to the beads is used. In some embodiments, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In some embodiments the ratio of cells to particles ranges from 1:100 to 100:1 and any integer values in-between and in some embodiments the ratio comprises 1:9 to 9:1 and any integer values in between, can also be used to stimulate T cells. The ratio of anti-CD3- and anti-CD28-coupled particles to T cells that result in T cell stimulation can vary as noted above, however certain suitable values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one suitable ratio being at least 1:1 particles per T cell. In some embodiments, a ratio of particles to cells of 1:1 or less is used. In some embodiments, a suitable particle: cell ratio is 1:5. In some embodiments, the ratio of particles to cells can be varied depending on the day of stimulation. For example, in some embodiments, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In some embodiments, the ratio of particles to cells is 1:1 on the first day of stimulation and adjusted to 1:5 on the third and fifth days of stimulation. In some embodiments, particles are added on a daily or every other day basis to a final ratio of 1:1 on the first day, and 1:5 on the third and fifth days of stimulation. In some embodiments, the ratio of particles to cells is 2:1 on the first day of stimulation and adjusted to 1:10 on the third and fifth days of stimulation. In some embodiments, particles are added on a daily or every other day basis to a final ratio of 1:1 on the first day, and 1:10 on the third and fifth days of stimulation. One of skill in the art will appreciate that a variety of other ratios may be suitable for use in the present disclosure. In particular, ratios will vary depending on particle size and on cell size and type. In some embodiments, the most typical ratios for use are in the neighborhood of 1:1, 2:1 and 3:1 on the first day.

In some embodiments, the cells, such as T cells, are combined with agent-coated beads, the beads and the cells are subsequently separated, and then the cells are cultured. In some embodiments, prior to culture, the agent-coated beads and cells are not separated but are cultured together. In some embodiments, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In some embodiments the cells (for example, 10⁴ to 10⁹ T cells) and beads (for example, Dynabeads® M-450 CD3/CD28 T paramagnetic beads at a ratio of 1:1) are combined in a buffer, for example PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present disclosure. In some embodiments, it may be desirable to significantly decrease the volume in which particles and cells are mixed together (i.e., increase the concentration of cells), to ensure maximum contact of cells and particles. For example, in some embodiments, a concentration of about 10 billion cells/ml, 9 billion/ml, 8 billion/ml, 7 billion/ml, 6 billion/ml, 5 billion/ml, or 2 billion cells/ml is used. In some embodiments, greater than 100 million cells/ml is used. In some embodiments, a concentration of cells of 10, 15, 20, 25, 30, 35, 40, 45, or 50 million cells/ml is used. In some embodiments, a concentration of cells from 75, 80, 85, 90, 95, or 100 million cells/ml is used. In some embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in some embodiments. For example, using high concentration of cells allows more efficient selection of CD8+ T cells that normally have weaker CD28 expression.

In some embodiments, cells transduced with a nucleic acid encoding a CAR, for example, a CAR described herein, for example, a CD19 CAR described herein, are expanded, for example, by a method described herein. In some embodiments, the cells are expanded in culture for a period of several hours (for example, about 2, 3, 4, 5, 6, 7, 8, 9, 10, 15, 18, 21 hours) to about 14 days (for example, 1, 2, 3, 4, 5, 6, 7, 8, 9, 10, 11, 12, 13 or 14 days). In some embodiments, the cells are expanded for a period of 4 to 9 days. In some embodiments, the cells are expanded for a period of 8 days or less, for example, 7, 6 or 5 days. In some embodiments, the cells are expanded in culture for 5 days, and the resulting cells are more potent than the same cells expanded in culture for 9 days under the same culture conditions. Potency can be defined, for example, by various T cell functions, for example proliferation, target cell killing, cytokine production, activation, migration, surface CAR expression, CAR quantitative PCR, or combinations thereof. In some embodiments, the cells, for example, a CD19 CAR cell described herein, expanded for 5 days show at least a one, two, three or four-fold increase in cells doublings upon antigen stimulation as compared to the same cells expanded in culture for 9 days under the same culture conditions. In some embodiments, the cells, for example, the cells expressing a CD19 CAR described herein, are expanded in culture for 5 days, and the resulting cells exhibit higher proinflammatory cytokine production, for example, IFN-γ and/or GM-CSF levels, as compared to the same cells expanded in culture for 9 days under the same culture conditions. In some embodiments, the cells, for example, a CD19 CAR cell described herein, expanded for 5 days show at least a one, two, three, four, five, ten-fold or more increase in pg/ml of proinflammatory cytokine production, for example, IFN-γ and/or GM-CSF levels, as compared to the same cells expanded in culture for 9 days under the same culture conditions.

Several cycles of stimulation may also be desired such that culture time of T cells can be 60 days or more. Conditions appropriate for T cell culture include an appropriate media (for example, Minimal Essential Media, α-MEM, RPMI Media 1640, AIM-V, DMEM, F-12, or X-vivo 15 (Lonza), X-Vivo 20, OpTmizer, and IMDM) that may contain factors necessary for proliferation and viability, including serum (for example, fetal bovine or human serum), interleukin-2 (IL-2), insulin, IFNγ, IL-4, IL-7, GM-CSF, IL-10, IL-12, IL-15, TGFβ, and TNFα or any other additives for the growth of cells known to the skilled artisan. Other additives for the growth of cells include, but are not limited to, surfactant, plasmanate, and reducing agents such as N-acetyl-cysteine and 2-mercaptoethanol. Media can include, but is not limited to RPMI 1640, AIM-V, DMEM, MEM, α-MEM, F-12, X-Vivo 15, X-Vivo 20, OpTmizer, and IMDM with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, for example, penicillin and streptomycin, are included only in experimental cultures, not in cultures of cells that are to be infused into a subject. The target cells are maintained under conditions necessary to support growth, for example, an appropriate temperature (for example, 37° C.) and atmosphere (for example, air plus 5% CO₂).

In some embodiments, the cells are expanded in an appropriate media (for example, media described herein) that includes one or more interleukin that result in at least a 200-fold (for example, 200-fold, 250-fold, 300-fold, 350-fold) increase in cells over a 14-day expansion period, for example, as measured by a method described herein such as flow cytometry. In some embodiments, the cells are expanded in the presence IL-15 and/or IL-7 (for example, IL-15 and IL-7).

In embodiments, methods described herein, for example, CAR-expressing cell manufacturing methods, comprise removing T regulatory cells, for example, CD25+ T cells or CD25^{high} T cells, from a cell population, for example, using an anti-CD25 antibody, or fragment thereof, or a CD25-binding ligand, IL-2. Methods of removing T regulatory cells, for example, CD25+ T cells or CD25^{high} T cells, from a cell population are described herein. In embodiments, the methods, for example, manufacturing methods, further comprise contacting a cell population (for example, a cell population in which T regulatory cells, such as CD25+ T cells or CD25^{high} T cells, have been depleted; or a cell population that has previously contacted an anti-CD25 antibody, fragment thereof, or CD25-binding ligand) with IL-15 and/or IL-7. For example, the cell population (for example, that has previously contacted an anti-CD25 antibody, fragment thereof, or CD25-binding ligand) is expanded in the presence of IL-15 and/or IL-7.

In some embodiments a CAR-expressing cell described herein is contacted with a composition comprising a interleukin-15 (IL-15) polypeptide, a interleukin-15 receptor alpha (IL-15Ra) polypeptide, or a combination of both a IL-15 polypeptide and a IL-15Ra polypeptide for example, hetIL-15, during the manufacturing of the CAR-expressing cell, for example, ex vivo. In embodiments, a CAR-expressing cell described herein is contacted with a composition comprising a IL-15 polypeptide during the manufacturing of the CAR-expressing cell, for example, ex vivo. In embodiments, a CAR-expressing cell described herein is contacted with a composition comprising a combination of both a IL-15 polypeptide and a IL-15 Ra polypeptide during the manufacturing of the CAR-expressing cell, for example, ex vivo. In embodiments, a CAR-expressing cell described herein is contacted with a composition comprising hetIL-15 during the manufacturing of the CAR-expressing cell, for example, ex vivo.

In some embodiments the CAR-expressing cell described herein is contacted with a composition comprising hetIL-15 during ex vivo expansion. In some embodiments, the CAR-expressing cell described herein is contacted with a composition comprising an IL-15 polypeptide during ex vivo expansion. In some embodiments, the CAR-expressing cell described herein is contacted with a composition comprising both an IL-15 polypeptide and an IL-15Ra polypeptide during ex vivo expansion. In some embodiments the contacting results in the survival and proliferation of a lymphocyte subpopulation, for example, CD8+ T cells.

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8+). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Once a CAR described herein is constructed, various assays can be used to evaluate the activity of the molecule, such as but not limited to, the ability to expand T cells following antigen stimulation, sustain T cell expansion in the absence of re-stimulation, and anti-cancer activities in appropriate in vitro and animal models. Assays to evaluate the effects of a CAR of the present disclosure are described in further detail below

Western blot analysis of CAR expression in primary T cells can be used to detect the presence of monomers and dimers, for example, as described in paragraph 695 of International Application WO2015/142675, filed Mar. 13, 2015, which is herein incorporated by reference in its entirety.

In vitro expansion of CAR+ T cells following antigen stimulation can be measured by flow cytometry. For example, a mixture of CD4+ and CD8+ T cells are stimulated with αCD3/αCD28 aAPCs followed by transduction with lentiviral vectors expressing GFP under the control of the promoters to be analyzed. Exemplary promoters include the CMV IE gene, EF-1α, ubiquitin C, or phosphoglycerokinase (PGK) promoters. GFP fluorescence is evaluated on day 6 of culture in the CD4⁺ and/or CD8⁺ T cell subsets by flow cytometry. See, for example, Milone et al., Molecular Therapy 17(8): 1453-1464 (2009). Alternatively, a mixture of CD4⁺ and CD8⁺ T cells are stimulated with αCD3/αCD28 coated magnetic beads on day 0, and transduced with CAR on day 1 using a bicistronic lentiviral vector expressing CAR along with eGFP using a 2A ribosomal skipping sequence. Cultures are re-stimulated with either a cancer associated antigen as described herein⁺ K562 cells (K562-expressing a cancer associated antigen as described herein), wild-type K562 cells (K562 wild type) or K562 cells expressing hCD32 and 4-1BBL in the presence of antiCD3 and anti-CD28 antibody (K562-BBL-3/28). Exogenous IL-2 is added to the cultures every other day at 100 IU/ml. GFP T cells are enumerated by flow cytometry using bead-based counting. See, for example, Milone et al., Molecular Therapy 17(8): 1453-1464 (2009).

Sustained CARP T cell expansion in the absence of re-stimulation can also be measured. See, for example, Milone et al., Molecular Therapy 17(8): 1453-1464 (2009). Briefly, mean T cell volume (fl) is measured on day 8 of culture using a Coulter Multisizer III particle counter or a higher version, a Nexcelom Cellometer Vision, Millipore Scepter or other cell counters, following stimulation with αCD3/αCD28 coated magnetic beads on day 0, and transduction with the indicated CAR on day 1.

Animal models can also be used to measure a CAR-expressing cell activity, for example, as described in paragraph 698 of International Application WO2015/142675, filed Mar. 13, 2015, which is herein incorporated by reference in its entirety.

Dose dependent CAR treatment response can be evaluated, for example, as described in paragraph 699 of International Application WO2015/142675, filed Mar. 13, 2015, which is herein incorporated by reference in its entirety.

Assessment of cell proliferation and cytokine production has been previously described, as described in paragraph 700 of International Application WO2015/142675, filed Mar. 13, 2015, which is herein incorporated by reference in its entirety.

Cytotoxicity can be assessed by a standard 51Cr-release assay, for example, as described in paragraph 701 of International Application WO2015/142675, filed Mar. 13, 2015, which is herein incorporated by reference in its entirety. Alternative non-radioactive methods can be utilized as well.

Cytotoxicity can also be assessed by measuring changes in adherent cell's electrical impedance, for example, using an xCELLigence real time cell analyzer (RTCA). In some embodiments, cytotoxicity is measured at multiple time points.

Imaging technologies can be used to evaluate specific trafficking and proliferation of CARs in tumor-bearing animal models, for example, as described in paragraph 702 of International Application WO2015/142675, filed Mar. 13, 2015, which is herein incorporated by reference in its entirety.

Other assays, including those described in the Example section herein as well as those that are known in the art can also be used to evaluate the CARs described herein.

Alternatively, or in combination to the methods disclosed herein, methods and compositions for one or more of: detection and/or quantification of CAR-expressing cells (for example, in vitro or in vivo (for example, clinical monitoring)); immune cell expansion and/or activation; and/or CAR-specific selection, that involve the use of a CAR ligand, are disclosed. In some embodiments, the CAR ligand is an antibody that binds to the CAR molecule, for example, binds to the extracellular antigen binding domain of CAR (for example, an antibody that binds to the antigen binding domain, for example, an anti-idiotypic antibody; or an antibody that binds to a constant region of the extracellular binding domain). In other embodiments, the CAR ligand is a CAR antigen molecule (for example, a CAR antigen molecule as described herein).

In some embodiments, a method for detecting and/or quantifying CAR-expressing cells is disclosed. For example, the CAR ligand can be used to detect and/or quantify CAR-expressing cells in vitro or in vivo (for example, clinical monitoring of CAR-expressing cells in a patient, or dosing a patient). The method includes:

providing the CAR ligand (optionally, a labelled CAR ligand, for example, a CAR ligand that includes a tag, a bead, a radioactive or fluorescent label);

acquiring the CAR-expressing cell (for example, acquiring a sample containing CAR-expressing cells, such as a manufacturing sample or a clinical sample);

contacting the CAR-expressing cell with the CAR ligand under conditions where binding occurs, thereby detecting the level (for example, amount) of the CAR-expressing cells present. Binding of the CAR-expressing cell with the CAR ligand can be detected using standard techniques such as FACS, ELISA and the like.

In some embodiments, a method of expanding and/or activating cells (for example, immune effector cells) is disclosed. The method includes:

providing a CAR-expressing cell (for example, a first CAR-expressing cell or a transiently expressing CAR cell);

contacting said CAR-expressing cell with a CAR ligand, for example, a CAR ligand as described herein), under conditions where immune cell expansion and/or proliferation occurs, thereby producing the activated and/or expanded cell population.

In certain embodiments, the CAR ligand is present on a substrate (for example, is immobilized or attached to a substrate, for example, a non-naturally occurring substrate). In some embodiments, the substrate is a non-cellular substrate. The non-cellular substrate can be a solid support chosen from, for example, a plate (for example, a microtiter plate), a membrane (for example, a nitrocellulose membrane), a matrix, a chip or a bead. In embodiments, the CAR ligand is present in the substrate (for example, on the substrate surface). The CAR ligand can be immobilized, attached, or associated covalently or non-covalently (for example, cross-linked) to the substrate. In some embodiments, the CAR ligand is attached (for example, covalently attached) to a bead. In the aforesaid embodiments, the immune cell population can be expanded in vitro or ex vivo. The method can further include culturing the population of immune cells in the presence of the ligand of the CAR molecule, for example, using any of the methods described herein.

In other embodiments, the method of expanding and/or activating the cells further comprises addition of a second stimulatory molecule, for example, CD28. For example, the CAR ligand and the second stimulatory molecule can be immobilized to a substrate, for example, one or more beads, thereby providing increased cell expansion and/or activation.

In some embodiments, a method for selecting or enriching for a CAR expressing cell is provided. The method includes contacting the CAR expressing cell with a CAR ligand as described herein; and selecting the cell on the basis of binding of the CAR ligand.

In yet other embodiments, a method for depleting, reducing and/or killing a CAR expressing cell is provided. The method includes contacting the CAR expressing cell with a CAR ligand as described herein; and targeting the cell on the basis of binding of the CAR ligand, thereby reducing the number, and/or killing, the CAR-expressing cell. In some embodiments, the CAR ligand is coupled to a toxic agent (for example, a toxin or a cell ablative drug). In some embodiments, the anti-idiotypic antibody can cause effector cell activity, for example, ADCC or ADC activities.

Exemplary anti-CAR antibodies that can be used in the methods disclosed herein are described, for example, in WO 2014/190273 and by Jena et al., “Chimeric Antigen Receptor (CAR)-Specific Monoclonal Antibody to Detect CD19-Specific T cells in Clinical Trials”, PLOS March 2013 8:3 e57838, the contents of which are incorporated by reference.

In some embodiments, the compositions and methods herein are optimized for a specific subset of T cells, for example, as described in US Serial No. PCT/US2015/043219 filed Jul. 31, 2015, the contents of which are incorporated herein by reference in their entirety. In some embodiments, the optimized subsets of T cells display an enhanced persistence compared to a control T cell, for example, a T cell of a different type (for example, CD8+ or CD4+) expressing the same construct.

In some embodiments, a CD4+ T cell comprises a CAR described herein, which CAR comprises an intracellular signaling domain suitable for (for example, optimized for, for example, leading to enhanced persistence in) a CD4+ T cell, for example, an ICOS domain. In some embodiments, a CD8+ T cell comprises a CAR described herein, which CAR comprises an intracellular signaling domain suitable for (for example, optimized for, for example, leading to enhanced persistence of) a CD8+ T cell, for example, a 4-1BB domain, a CD28 domain, or another costimulatory domain other than an ICOS domain. In some embodiments, the CAR described herein comprises an antigen binding domain described herein, for example, a CAR comprising an antigen binding domain.

In some embodiments, described herein is a method of treating a subject, for example, a subject having cancer. The method includes administering to said subject, an effective amount of:

1) a CD4+ T cell comprising a CAR (the CARCD4+) comprising:

an antigen binding domain, for example, an antigen binding domain described herein;

a transmembrane domain; and

an intracellular signaling domain, for example, a first costimulatory domain, for example, an ICOS domain; and

2) a CD8+ T cell comprising a CAR (the CARCD8+) comprising:

an antigen binding domain, for example, an antigen binding domain described herein;

a transmembrane domain; and

an intracellular signaling domain, for example, a second costimulatory domain, for example, a 4-1BB domain, a CD28 domain, or another costimulatory domain other than an ICOS domain;

wherein the CARCD4+ and the CARCD8+ differ from one another.

Optionally, the method further includes administering:

3) a second CD8+ T cell comprising a CAR (the second CARCD8+) comprising:

an antigen binding domain, for example, an antigen binding domain described herein;

a transmembrane domain; and

an intracellular signaling domain, wherein the second CARCD8+ comprises an intracellular signaling domain, for example, a costimulatory signaling domain, not present on the CARCD8+, and, optionally, does not comprise an ICOS signaling domain.

### Biopolymer Delivery Methods

In some embodiments, one or more CAR-expressing cells as disclosed herein can be administered or delivered to the subject via a biopolymer scaffold, for example, a biopolymer implant. Biopolymer scaffolds can support or enhance the delivery, expansion, and/or dispersion of the CAR-expressing cells described herein. A biopolymer scaffold comprises a biocompatible (for example, does not substantially induce an inflammatory or immune response) and/or a biodegradable polymer that can be naturally occurring or synthetic. Exemplary biopolymers are described, for example, in paragraphs 1004-1006 of International Application WO2015/142675, filed Mar. 13, 2015, which is herein incorporated by reference in its entirety.

### Pharmaceutical Compositions and Treatments

In some embodiments, this disclosure provides a method of treating a patient, comprising administering CAR-expressing cells produced as described herein, optionally in combination with one or more other therapies. In some embodiments, the CAR-expressing cells express a CCAR disclosed herein. In some embodiments, the CAR-expressing cells express a CAR disclosed herein and a regulatory molecule disclosed herein. In some embodiments, this disclosure provides a method of treating a patient, comprising administering a reaction mixture comprising CAR-expressing cells as described herein, optionally in combination with one or more other therapies. In some embodiments, this disclosure provides a method of shipping or receiving a reaction mixture comprising CAR-expressing cells as described herein. In some embodiments, this disclosure provides a method of treating a patient, comprising receiving a CAR-expressing cell that was produced as described herein, and further comprising administering the CAR-expressing cell to the patient, optionally in combination with one or more other therapies. In some embodiments, this disclosure provides a method of treating a patient, comprising producing a CAR-expressing cell as described herein, and further comprising administering the CAR-expressing cell to the patient, optionally in combination with one or more other therapies. The other therapy may be, for example, a cancer therapy such as chemotherapy.

In some embodiments, cells expressing a CAR described herein are administered to a subject in combination with a molecule that decreases the Treg cell population. Methods that decrease the number of (for example, deplete) Treg cells are known in the art and include, for example, CD25 depletion, cyclophosphamide administration, modulating GITR function. Without wishing to be bound by theory, it is believed that reducing the number of Treg cells in a subject prior to apheresis or prior to administration of a CAR-expressing cell described herein reduces the number of unwanted immune cells (for example, Tregs) in the tumor microenvironment and reduces the subject's risk of relapse.

In some embodiments, a therapy described herein, for example, a CAR-expressing cell, is administered to a subject in combination with a molecule targeting GITR and/or modulating GITR functions, such as a GITR agonist and/or a GITR antibody that depletes regulatory T cells (Tregs). In embodiments, cells expressing a CAR described herein are administered to a subject in combination with cyclophosphamide. In some embodiments, the GITR binding molecules and/or molecules modulating GITR functions (for example, GITR agonist and/or Treg depleting GITR antibodies) are administered prior to the CAR-expressing cell. For example, in some embodiments, a GITR agonist can be administered prior to apheresis of the cells. In embodiments, cyclophosphamide is administered to the subject prior to administration (for example, infusion or re-infusion) of the CAR-expressing cell or prior to apheresis of the cells. In embodiments, cyclophosphamide and an anti-GITR antibody are administered to the subject prior to administration (for example, infusion or re-infusion) of the CAR-expressing cell or prior to apheresis of the cells. In some embodiments, the subject has cancer (for example, a solid cancer or a hematological cancer such as ALL or CLL). In some embodiments, the subject has CLL. In embodiments, the subject has ALL. In embodiments, the subject has a solid cancer, for example, a solid cancer described herein. Exemplary GITR agonists include, for example, GITR fusion proteins and anti-GITR antibodies (for example, bivalent anti-GITR antibodies) such as, for example, a GITR fusion protein described in U.S. Pat. No. 6,111,090, European Patent No.: 090505B1, U.S Pat. No. 8,586,023, PCT Publication Nos.: WO 2010/003118 and 2011/090754, or an anti-GITR antibody described, for example, in U.S. Pat. No. 7,025,962, European Patent No.: 1947183B1, U.S. Pat. Nos. 7,812,135, 8,388,967, 8,591,886, European Patent No.: EP 1866339, PCT Publication No.: WO 2011/028683, PCT Publication No.: WO 2013/039954, PCT Publication No.: WO2005/007190, PCT Publication No.: WO 2007/133822, PCT Publication No.: WO2005/055808, PCT Publication No.: WO 99/40196, PCT Publication No.: WO 2001/03720, PCT Publication No.: WO99/20758, PCT Publication No.: WO2006/083289, PCT Publication No.: WO 2005/115451, U.S. Pat. No. 7,618,632, and PCT Publication No.: WO 2011/051726.

In some embodiments, a CAR expressing cell described herein is administered to a subject in combination with a GITR agonist, for example, a GITR agonist described herein. In some embodiments, the GITR agonist is administered prior to the CAR-expressing cell. For example, in some embodiments, the GITR agonist can be administered prior to apheresis of the cells. In some embodiments, the subject has CLL.

The methods described herein can further include formulating a CAR-expressing cell in a pharmaceutical composition. Pharmaceutical compositions may comprise a CAR-expressing cell, for example, a plurality of CAR-expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (for example, aluminum hydroxide); and preservatives. Compositions can be formulated, for example, for intravenous administration.

In some embodiments, the pharmaceutical composition is substantially free of, for example, there are no detectable levels of a contaminant, for example, selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In some embodiments, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an immunologically effective amount,” “an anti-cancer effective amount,” “a cancer-inhibiting effective amount,” or “therapeutic amount” is indicated, the precise amount of the compositions to be administered can be determined by a physician with consideration of individual differences in age, weight, tumor size, extent of infection or metastasis, and condition of the patient (subject). It can generally be stated that a pharmaceutical composition comprising the immune effector cells (for example, T cells, NK cells) described herein may be administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, for example, Rosenberg et al., New Eng. J. of Med. 319:1676, 1988).

In some embodiments, a dose of CAR cells (for example, CD19 CAR cells) comprises about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (for example, CD19 CAR cells) comprises at least about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (for example, CD19 CAR cells) comprises up to about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (for example, CD19 CAR cells) comprises about 1.1×10⁶-1.8×10⁷ cells/kg. In some embodiments, a dose of CAR cells (for example, CD19 CAR cells) comprises about 1×10⁷, 2×10⁷, 5×10⁷, 1×10⁸, 2×10⁸, 5×10⁸, 1×10⁹, 2×10⁹, or 5×10⁹ cells. In some embodiments, a dose of CAR cells (for example, CD19 CAR cells) comprises at least about 1×10⁷, 2×10⁷, 5×10⁷, 1×10⁸, 2×10⁸, 5×10⁸, 1×10⁹, 2×10⁹, or 5×10⁹ cells. In some embodiments, a dose of CAR cells (for example, CD19 CAR cells) comprises up to about 1×10⁷, 2×10⁷, 5×10⁷, 1×10⁸, 2×10⁸, 5×10⁸, 1×10⁹, 2×10⁹, or 5×10⁹ cells.

In some embodiments, it may be desired to administer activated immune effector cells (for example, T cells, NK cells) to a subject and then subsequently redraw blood (or have an apheresis performed), activate immune effector cells (for example, T cells, NK cells) therefrom, and reinfuse the patient with these activated and expanded immune effector cells (for example, T cells, NK cells). This process can be carried out multiple times every few weeks. In some embodiments, immune effector cells (for example, T cells, NK cells) can be activated from blood draws of from 10 cc to 400 cc. In some embodiments, immune effector cells (for example, T cells, NK cells) are activated from blood draws of 20 cc, 30 cc, 40 cc, 50 cc, 60 cc, 70 cc, 80 cc, 90 cc, or 100 cc.

The administration of the subject compositions may be carried out in any convenient manner. The compositions described herein may be administered to a patient trans arterially, subcutaneously, intradermally, intratumorally, intranodally, intramedullary, intramuscularly, by intravenous (i.v.) injection, or intraperitoneally, for example, by intradermal or subcutaneous injection. The compositions of immune effector cells (for example, T cells, NK cells) may be injected directly into a tumor, lymph node, or site of infection.

### Dosage Regimen

In some embodiments, a dose of viable CAR-expressing cells (for example, viable CD19, BCMA, CD20, or CD22 CAR-expressing cells) comprises about 0.5×10⁶ viable CAR-expressing cells to about 1.25×10⁹ viable CAR-expressing cells (for example, 0.5×10⁶ viable CAR-expressing cells to 1.25×10⁹ viable CAR-expressing cells). In some embodiments, a dose of viable CAR-expressing cells (for example, viable CD19, BCMA, CD20, or CD22 CAR-expressing cells) comprises about 1×10⁶, about 2.5×10⁶, about 5×10⁶, about 1.25×10⁷, about 2.5×10⁷, about 5×10⁷, about 5.75×10⁷, or about 8×10⁷ viable CAR-expressing cells.

### Patient Selection

In some embodiments of any of the methods of treating a subject, or composition for use disclosed herein, the subject has a cancer, for example, a hematological cancer. In some embodiments, the cancer is chosen from lymphocytic leukemia (CLL), mantle cell lymphoma (MCL), multiple myeloma, acute lymphoid leukemia (ALL), Hodgkin lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitts lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+ large B-cell lymphoma, large B-cell lymphoma arising in HHV8-associated multicentric Castleman disease, primary effusion lymphoma, B-cell lymphoma, acute myeloid leukemia (AML), or unclassifiable lymphoma. In some embodiments, the cancer is a relapsed and/or refractory cancer.

In some embodiments of any of the methods of treating a subject, or composition for use disclosed herein, the subject has CLL or SLL. In some embodiments, the subject having CLL or SLL has previously been administered a BTK inhibitor therapy, for example, ibrutinib, for least 1-12 months, for example, 6 months. In some embodiments, the BTK inhibitor therapy, for example, ibrutinib therapy, is a second line therapy. In some embodiments, the subject had a partial response, or had stable disease in response to the BTK inhibitor therapy. In some embodiments, the subject did not response to the BTK inhibitor therapy. In some embodiments, the subject developed resistance, for example, developed ibrutinib resistance mutations. In some embodiments, the ibrutinib resistance mutations comprise a mutation in the gene encoding BTK and/or the gene encoding PLCg2. In some embodiments, the subject is an adult, for example, at least 18 years of age.

In some embodiments of any of the methods of treating a subject, or composition for use disclosed herein, the subject has DLBCL, for example, relapsed and/or refractory DLBCL. In some embodiments, the subject having DLBCL, for example, relapsed and/or refractory DLBCL, has previously been administered at least 2 lines of chemotherapy, for example, an anti-CD20 therapy and/or an anthracycline-based chemotherapy. In some embodiments, the subject has previously received stem cell therapy, for example, autologous stem cell therapy, and has not responded to said stem cell therapy. In some embodiments, the subject is not eligible for stem cell therapy, for example, autologous stem cell therapy. In some embodiments, the subject is an adult, for example, at least 18 years of age.

### Biomarkers for Evaluating CAR-Effectiveness

In some embodiments, disclosed herein is a method of evaluating or monitoring the effectiveness of a CAR-expressing cell therapy (for example, a CD19 or BCMA CAR therapy), in a subject (for example, a subject having a cancer, for example, a hematological cancer). The method includes acquiring a value of effectiveness to the CAR therapy, wherein said value is indicative of the effectiveness or suitability of the CAR-expressing cell therapy.

In embodiments, the value of effectiveness to the CAR therapy in a subject having CLL or SLL, comprises a measure of one, two, three, or all of the following parameters:

(i) a mutation in a gene encoding BTK in a sample (for example, an apheresis sample or a manufactured CAR-expressing cell product sample);

(ii) a mutation in a gene encoding PLCg2 in a sample (for example, an apheresis sample or a manufactured CAR-expressing cell product sample);

(iii) minimal residual disease, for example, as evaluated by the level and/or activity of CD8, CD4, CD3, CD5, CD19, CD20, CD22, CD43, CD79b, CD27, CD45RO, CD45RA, CCR7, CD95, Lag3, PD-1, Tim-3, and/or CD81; or as evaluated by immunoglobulin deep sequencing; in a sample (for example, an apheresis sample or tumor sample from the subject); or

(iv) the level or activity of one, two, three, four, five, six, seven, eight, nine, ten or all of the cytokines chosen from IFN-g, IL-2, IL-4, IL-6, IL-8, IL-10, IL-15, TNF-a, IP-10, MCP1, MIP1a, in a sample, for example, an apheresis sample from the subject.

In embodiments, the value of effectiveness to the CAR therapy in a subject having DLBCL, for example, relapsed and/or refractory DLBCL, comprises a measure of one or both the following parameters:

(i) minimal residual disease, for example, as evaluated by the level and/or activity of CD8, CD4, CAR19, CD3, CD27, CD45RO, CD45RA, CCR7, CD95, Lag3, PD-1, and/or Tim-3; or as evaluated by immunoglobulin deep sequencing; in a sample (for example, an apheresis sample or tumor sample from the subject); or

(ii) the level or activity of one, two, three, four, five, six, seven, eight, nine, ten or all of the cytokines chosen from IFN-g, IL-2, IL-4, IL-6, IL-8, IL-10, IL-15, TNF-a, IP-10, MCP1, MIP1a, in a sample (for example, an apheresis sample from the subject).

In other embodiments, the value of effectiveness to the CAR therapy, further comprises a measure of one, two, three, four, five, six or more (all) of the following parameters:

(i) the level or activity of one, two, three, or more (for example, all) of resting T_{EFF} cells, resting T_{REG} cells, younger T cells (for example, naïve T cells (for example, naïve CD4 or CD8 T cells, naïve gamma/delta T cells), or stem memory T cells (for example, stem memory CD4 or CD8 T cells, or stem memory gamma/delta T cells), or early memory T cells, or a combination thereof, in a sample (for example, an apheresis sample or a manufactured CAR-expressing cell product sample);

(ii) the level or activity of one, two, three, or more (for example, all) of activated T_{EFF} cells, activated T_{REG} cells, older T cells (for example, older CD4 or CD8 cells), or late memory T cells, or a combination thereof, in a sample (for example, an apheresis sample or a manufactured CAR-expressing cell product sample);

(iii) the level or activity of an immune cell exhaustion marker, for example, one, two or more immune checkpoint inhibitors (for example, PD-1, PD-L1, TIM-3, TIGIT and/or LAG-3) in a sample (for example, an apheresis sample or a manufactured CAR-expressing cell product sample). In some embodiments, an immune cell has an exhausted phenotype, for example, co-expresses at least two exhaustion markers, for example, co-expresses PD-1 and TIM-3. In other embodiments, an immune cell has an exhausted phenotype, for example, co-expresses at least two exhaustion markers, for example, co-expresses PD-1 and LAG-3;

(iv) the level or activity of CD27 and/or CD45RO− (for example, CD27+ CD45RO−) immune effector cells, for example, in a CD4+ or a CD8+ T cell population, in a sample (for example, an apheresis sample or a manufactured CAR-expressing cell product sample);

(v) the level or activity of one, two, three, four, five, six, seven, eight, nine, ten, eleven or all of the biomarkers chosen from CCL20, IL-17a, IL-6, PD-1, PD-L1, LAG-3, TIM-3, CD57, CD27, CD122, CD62L, KLRG1;

(vi) a cytokine level or activity (for example, quality of cytokine reportoire) in a CAR-expressing cell product sample, for example, CLL-1-expressing cell product sample; or

(vii) a transduction efficiency of a CAR-expressing cell in a manufactured CAR-expressing cell product sample.

In some embodiments of any of the methods disclosed herein, the CAR-expressing cell therapy comprises a plurality (for example, a population) of CAR-expressing immune effector cells, for example, a plurality (for example, a population) of T cells or NK cells, or a combination thereof. In some embodiments, the CAR-expressing cell therapy is a CD19 CAR therapy.

In some embodiments of any of the methods disclosed herein, the measure of one or more of the parameters disclosed herein is obtained from an apheresis sample acquired from the subject. The apheresis sample can be evaluated prior to infusion or re-infusion.

In some embodiments of any of the methods disclosed herein, the measure of one or more of the parameters disclosed herein is obtained from a tumor sample acquired from the subject.

In some embodiments of any of the methods disclosed herein, the measure of one or more of the parameters disclosed herein is obtained from a manufactured CAR-expressing cell product sample, for example, CD19 CAR-expressing cell product sample. The manufactured CAR-expressing cell product can be evaluated prior to infusion or re-infusion.

In some embodiments of any of the methods disclosed herein, the subject is evaluated prior to receiving, during, or after receiving, the CAR-expressing cell therapy.

In some embodiments of any of the methods disclosed herein, the measure of one or more of the parameters disclosed herein evaluates a profile for one or more of gene expression, flow cytometry or protein expression.

In some embodiments of any of the methods disclosed herein, the method further comprises identifying the subject as a responder, a non-responder, a relapser or a non-relapser, based on a measure of one or more of the parameters disclosed herein.

In some embodiments of any of the methods disclosed herein, a responder, for example, complete responder has, or is identified as having, a greater, for example, a statistically significant

greater, percentage of CD8+ T cells compared to a reference value, for example, a non-responder percentage of CD8+ T cells.

In some embodiments of any of the methods disclosed herein, a responder, for example, complete responder has, or is identified as having, a greater percentage of CD27+ CD45RO− immune effector cells, for example, in the CD8+ population, compared to a reference value, for example, a non-responder number of CD27+ CD45RO− immune effector cells.

In some embodiments of any of the methods disclosed herein, a responder, for example, complete responder or a partial responder has, or is identified as having, a greater, for example, a statistically significant greater, percentage of CD4+ T cells compared to a reference value, for example, a non-responder percentage of CD4+ T cells.

In some embodiments of any of the methods disclosed herein, a responder, for example, complete responder has, or is identified as having, a greater percentage of one, two, three, or more (for example, all) of resting T_{EFF} cells, resting T_{REG} cells, younger T cells, or early memory T cells, or a combination thereof, compared to a reference value, for example, a non-responder number of resting T_{EFF} cells, resting T_{REG} cells, younger T cells, or early memory T cells.

In some embodiments of any of the methods disclosed herein, a non-responder has, or is identified as having, a greater percentage of one, two, three, or more (for example, all) of activated T_{EFF} cells, activated T_{REG} cells, older T cells (for example, older CD4 or CD8 cells), or late memory T cells, or a combination thereof, compared to a reference value, for example, a responder number of activated T_{EFF} cells, activated T_{REG} cells, older T cells (for example, older CD4 or CD8 cells), or late memory T cells.

In some embodiments of any of the methods disclosed herein, a non-responder has, or is identified as having, a greater percentage of an immune cell exhaustion marker, for example, one, two or more immune checkpoint inhibitors (for example, PD-1, PD-L1, TIM-3, TIGIT, and/or LAG-3). In some embodiments, a non-responder has, or is identified as having, a greater percentage of PD-1, PD-L1, or LAG-3 expressing immune effector cells (for example, CD4+ T cells and/or CD8+ T cells) (for example, CAR-expressing CD4+ cells and/or CD8+ T cells) compared to the percentage of PD-1 or LAG-3 expressing immune effector cells from a responder.

In some embodiments, a non-responder has, or is identified as having, a greater percentage of immune cells having an exhausted phenotype, for example, immune cells that co-express at least two exhaustion markers, for example, co-expresses PD-1, PD-L1 and/or TIM-3. In other embodiments, a non-responder has, or is identified as having, a greater percentage of immune cells having an exhausted phenotype, for example, immune cells that co-express at least two exhaustion markers, for example, co-expresses PD-1 and LAG-3.

In some embodiments of any of the methods disclosed herein, a non-responder has, or is identified as having, a greater percentage of PD-1/PD-L1+/LAG-3+ cells in the CAR-expressing cell population (for example, a CLL-1 CAR+ cell population) compared to a responder (for example, a complete responder) to the CAR-expressing cell therapy.

In some embodiments of any of the methods disclosed herein, the responder (for example, the complete or partial responder) has one, two, three or more (or all) of the following profile:

(i) has a greater number of CD27+ immune effector cells compared to a reference value, for example, a non-responder number of CD27+ immune effector cells;

(ii) has a greater number of CD8+ T cells compared to a reference value, for example, a non-responder number of CD8+ T cells;

(iii) has a lower number of immune cells expressing one or more checkpoint inhibitors, for example, a checkpoint inhibitor chosen from PD-1, PD-L1, LAG-3, TIM-3, or KLRG-1, or a combination, compared to a reference value, for example, a non-responder number of cells expressing one or more checkpoint inhibitors; or

(iv) has a greater number of one, two, three, four or more (all) of resting T_{EFF} cells, resting T_{REG} cells, naïve CD4 cells, unstimulated memory cells or early memory T cells, or a combination thereof, compared to a reference value, for example, a non-responder number of resting T_{EFF} cells, resting T_{REG} cells, naïve CD4 cells, unstimulated memory cells or early memory T cells.

In embodiments, a subject who is a responder, a non-responder, a relapser or a non-relapser identified by the methods herein can be further evaluated according to clinical criteria. For example, a complete responder has, or is identified as, a subject having a disease, for example, a cancer, who exhibits a complete response, for example, a complete remission, to a treatment. A complete response may be identified, for example, using the NCCN Guideline®, or the International Workshop on Chronic Lymphocytic Leukemia (iwCLL) 2018 guidelines as disclosed in Hallek M et al., Blood (2018) 131:2745-2760 “iwCLL guidelines for diagnosis, indications for treatment, response assessment, and supportive management of CLL,” the entire contents of which are hereby incorporated by reference in its entirety. A partial responder has, or is identified as, a subject having a disease, for example, a cancer, who exhibits a partial response, for example, a partial remission, to a treatment. A partial response may be identified, for example, using the NCCN Guidelines®, or iwCLL 2018 criteria as described herein. A non-responder has, or is identified as, a subject having a disease, for example, a cancer, who does not exhibit a response to a treatment, for example, the patient has stable disease or progressive disease. A non-responder may be identified, for example, using the NCCN Guidelines®, or iwCLL 2018 criteria as described herein.

Alternatively, or in combination with the methods disclosed herein, responsive to said value, performing one, two, three four or more of:

administering for example, to a responder or a non-relapser, a CAR-expressing cell therapy;

administered an altered dosing of a CAR-expressing cell therapy;

altering the schedule or time course of a CAR-expressing cell therapy;

administering, for example, to a non-responder or a partial responder, an additional agent in combination with a CAR-expressing cell therapy, for example, a checkpoint inhibitor, for example, a checkpoint inhibitor described herein;

administering to a non-responder or partial responder a therapy that increases the number of younger T cells in the subject prior to treatment with a CAR-expressing cell therapy;

modifying a manufacturing process of a CAR-expressing cell therapy, for example, enriching for younger T cells prior to introducing a nucleic acid encoding a CAR, or increasing the transduction efficiency, for example, for a subject identified as a non-responder or a partial responder;

administering an alternative therapy, for example, for a non-responder or partial responder or relapser; or

if the subject is, or is identified as, a non-responder or a relapser, decreasing the T_{REG} cell population and/or T_{REG} gene signature, for example, by one or more of CD25 depletion, administration of cyclophosphamide, anti-GITR antibody, or a combination thereof.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

### Generation of CARTs with Cytokine Stimulation

### Summary

This example describes a CART manufacturing process called “cytokine process.” In some embodiments, cells (for example, T cells) are seeded in media (for example, serum-containing media, for example, media containing 2% serum). One or more cytokines (for example, one or more cytokines chosen from IL-2, IL-7, IL-15 (for example, hetIL-15 (IL15/sIL-15Ra)), IL-21, or IL-6 (for example, IL-6/sIL-6Ra) as well as vectors (for example, lentiviral vectors) encoding a CAR are added to the cells. After incubation for 20-24 hours, cells are washed, formulated, and cryopreserved. Exemplary cytokine process is shown in FIG. 1A.

Compared to the traditional CART manufacturing process, this revised process eliminates CD3/CD28 stimulation as well as ex vivo T cell expansion. Without wishing to be bound by theory, anti-CD3/anti-CD28 beads drive differentiation into central memory cells; and in contrast, cytokines such as IL-15, IL-21, and IL-7 may help preserve the undifferentiated phenotype of transduced CD3+ T cells. As a consequence, the cytokine process which does not involve CD3/CD28 activation may generate CART cells with a higher percentage of naïve/stem T cells, compared to CART cells generated using the traditional approach.

### Methods

After obtaining an apheresis within 24 hours of collection, T cells were purified and the purity of the T-cells obtained was assessed by flow cytometry. The T cells were frozen and placed in the liquid nitrogen until required for use.

Alternatively, a cryopreserved apheresis sample is prepared and enriched for CD4+ T cells and/or CD8+ T cells using a Prodigy® machine.

IL-7 and IL-15 were prepared at 1,000 folds of the final concentration required. IL-2 was prepared by a 10-fold dilution in media.

TABLE 19
Cytokine conditions

| | Conditions |
|---|---|
| 1. | IL2 |
| 2. | IL-7 |
| 3. | IL-15 |
| 4. | IL2 + IL7 |
| 5. | IL-7 + IL-15 |
| 6. | IL2 + IL-15 |
| 7. | Beads + IL2 |
| 8. | Beads + IL15 |

In the expander bead stimulated conditions, calculations were performed to plate cells with a final concentration of bead to cell ratio of 3:1. The Dynabeads® magnetic beads were washed twice using a Dynamag® and resuspended in the required volume of media for the experiment. The washed beads were added to the tubes that contained the specific cytokines and cells.

At the time of plating, the cells were transduced with a lentiviral vector with a multiplicity of infection (MOI) of 1. The specific volume of vector to be transduced was calculated based on the multiplicity of infection (MOI) and concentration (titer) of the vector lot in use. The titer and the MOI were measured based on primary T cell lines.

In the conditions where cytokines alone were utilized for stimulation, the cells were resuspended post wash at a concentration of 1E7/ml and added to a conical tube that already contained the cytokines depending on the condition (Table 19). After the cells and cytokines were added the lentiviral vector was added followed by the media.

In all of the conditions the cells were mixed and lml was plated in 14 wells of a 24 well plate. The cells were placed in an incubator that was at 37° C. and 5% CO₂.

On the following day the cells were harvested, the concentration and viability of the cells was noted. Their function was measured using a cytotoxicity and proliferation (EDU) incorporation assay. These cells were referred to as “day 1 CARTs.”

The cells were immunophenotyped for T cell differentiation status and transduction of the CAR was assessed using flow cytometry. The cells were washed, viability dye was added followed by the antibody cocktail (Table 20), and the plates were incubated for 20 minutes at room temperature. After the incubation, the cells were washed twice and fixed prior to being analyzed on the BD fortessa.

TABLE 20
Antigens of the panel of antibodies used to determine
the differentiation status of the T-cells
Antigen

Viability
CD3
CD4
CD8
HLADR
CD28
CD45RO
CD95
CCR7
Anti-Idiotype

To determine if the day 1 CARTs still maintained the ability to expand post-harvest, 5e6 cells/condition were expanded using CD3/CD28 beads in a T25 flask at a ratio of 3:1 (beads to cells). The Dynabeads® magnetic beads were washed as previously described. The media contained no cytokines. The cells were placed in an incubator that was at 37° C. and 5% CO₂.

In the case of the T cells expanded with the CD3/CD28 beads every 2 days, the cells were counted and spilt up to 10 days in culture. On day 10 the cells were harvested, counted, immunophenotyped using the differentiation panel (Table 20) and frozen in Cryostor 10™. The cells were thawed for functional assays that included cytotoxicity assay, proliferation assay and cytokine secretion assay.

The cells expanded in the presence of CD3/CD28 beads in vitro for 10 days were referred to as “day 10 CARTs.”

### Results

When purified T cells were incubated with cytokines in the absence of any other activation stimulus, there was an increase in transduction from day 1 to day 4 (FIG. 1B). Independent of the time point and cytokine condition, the predominant population within the CAR positive population was naïve (FIGS. 1D, 1E, and 1F). The elimination of the activation agent led to an enhancement of transduction with the primitive population. Notably, exposure to IL-2 or IL-15 maintained self-renewing T cells in vitro (FIG. 1G). Similar phenomenon was observed under the other cytokine treatments tested (IL-7; IL2+IL7; IL-7+IL-15; and IL2+IL-15) (data not shown). The cytokine process (using IL2 or IL-15 in this specific example) maintained or slightly increased the percentage of CD45RO−CCR7+ cells (FIG. 1G). Similar data are shown in FIGS. 1H and 1I for IL-2, IL-15, and a combination of IL-7 and IL-15. Culturing T cells with the indicated cytokines for 24 hours maintained the naïve phenotype of CD3+ T cells, and reduced the percentage of central memory T cells (FIGS. 1H and 1I).

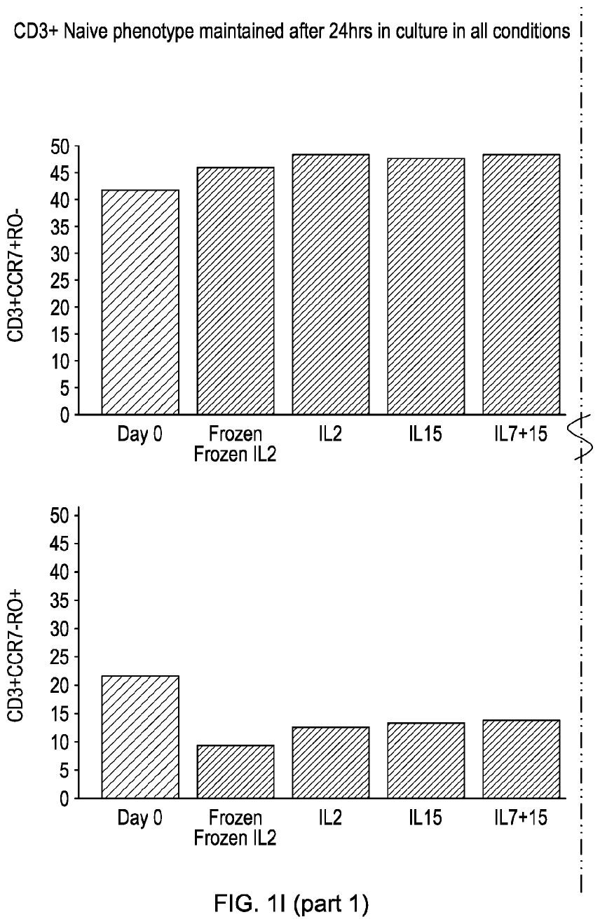

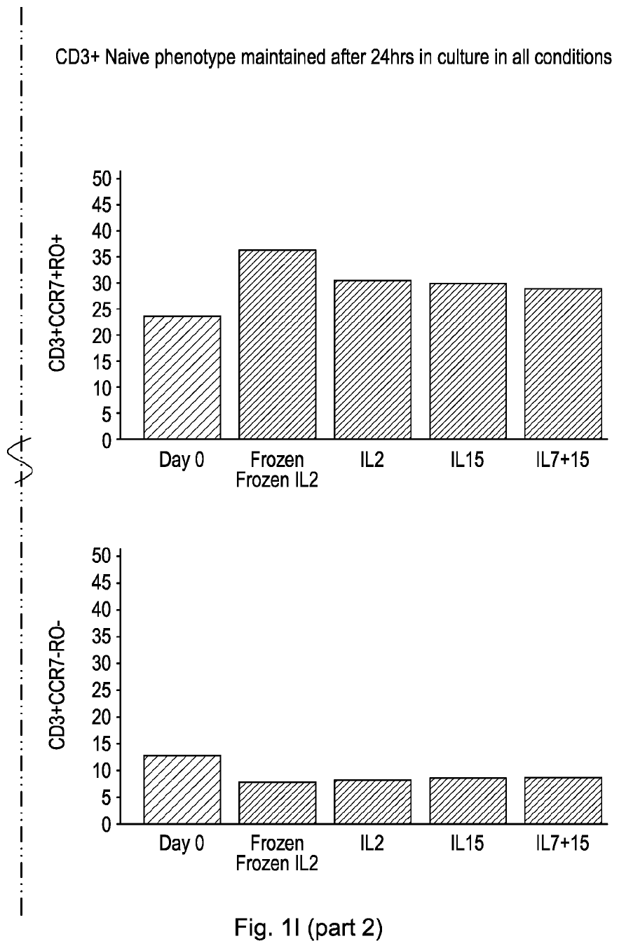

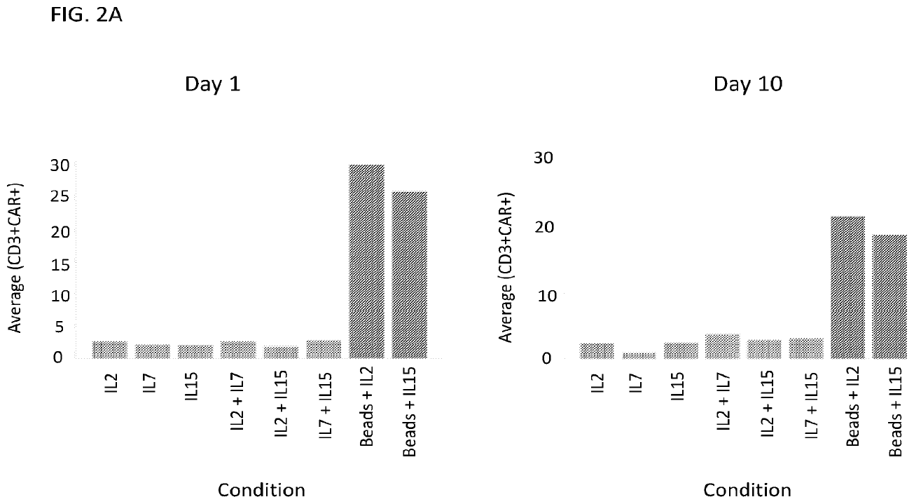

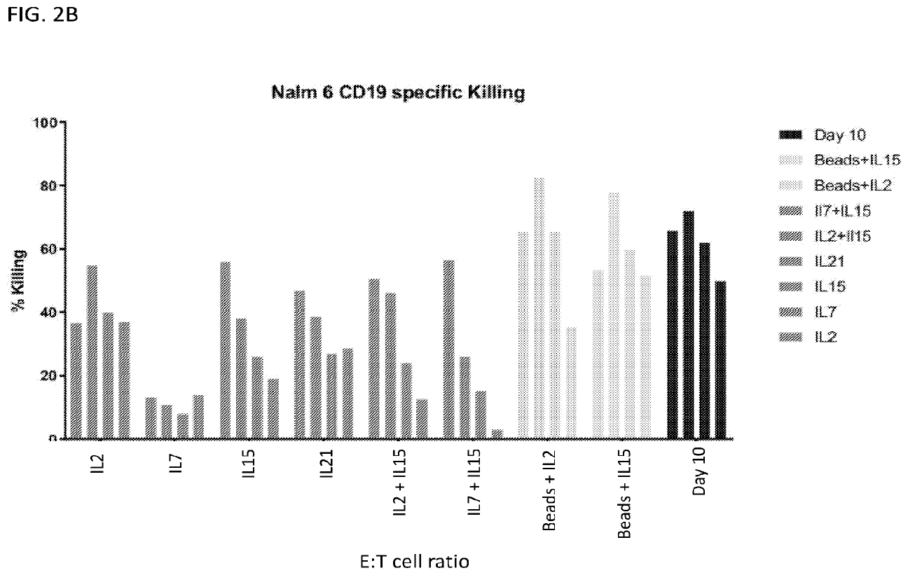

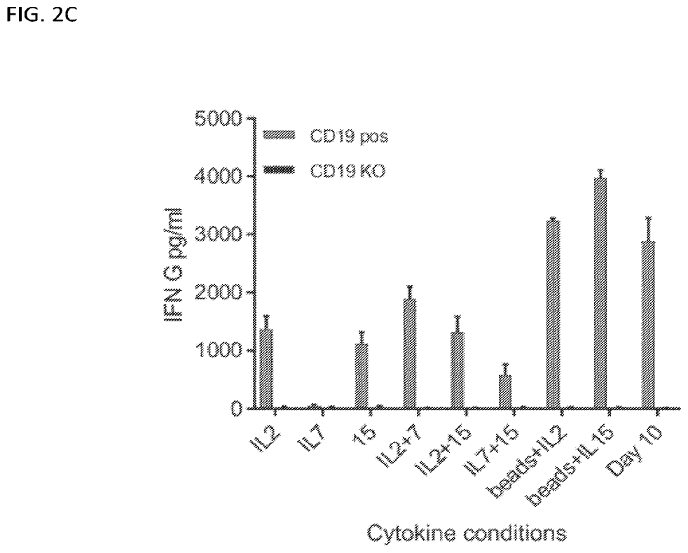

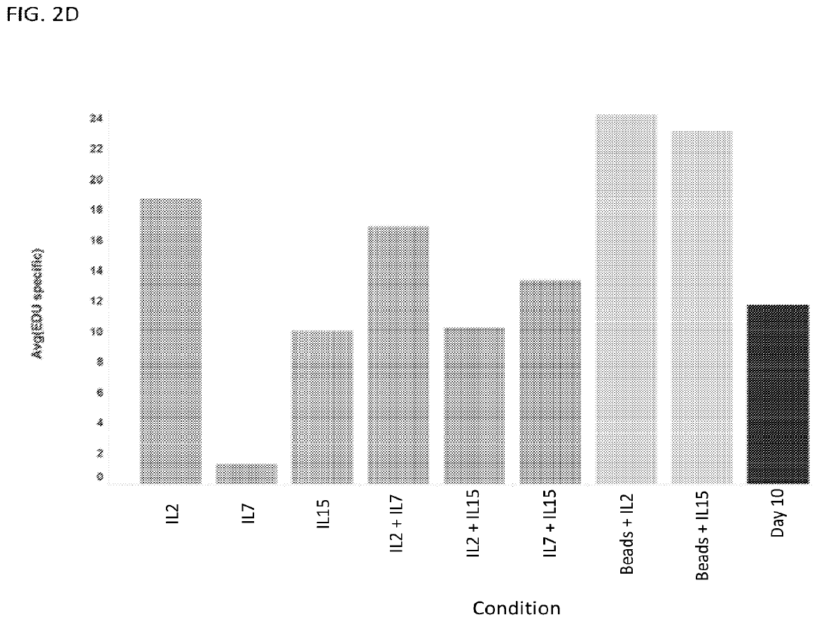

To ensure that the transduction observed within 24 hours was stable, the CARTs generated within 24 hours were washed to remove any residual virus and expanded over 10 days using CD3/D28 expansion beads. The expanded cells demonstrated almost equivalent transduction to the day 1 CARTs indicating that the transduction was stable (FIG. 2A).

The functionality of the day 1 CARTs and day 10 CARTs was tested using a cytotoxicity, a cytokine release, and a proliferation assay. The target cells were Nalm6 cells, a B cell ALL cell line that expresses CD19. The cytotoxicity assay demonstrated that the day 1 CARTs post expansion were equivalent at killing as compared to the day 10 CARTs (FIG. 2B) even though the day 1 CARTs had much fewer transduced cells. The same day 1 CARTs that had been expanded were compared for the secretion for IFN-gamma and found to have a lower secretion of IFN-gamma as compared to the day 10 CARTs (FIG. 2C), which was likely due to the difference in the number of transduced cells. In separate studies where the day 1 CARTs had a higher level of transduction, they secreted a higher level of IFN-gamma (data not shown). Furthermore, the day 1 CARTs from all the treatment conditions except the IL7-only condition showed similar or higher proliferation than the day 10 CARTs (FIG. 2D). The data shown in FIG. 2D were not normalized for transduction levels.

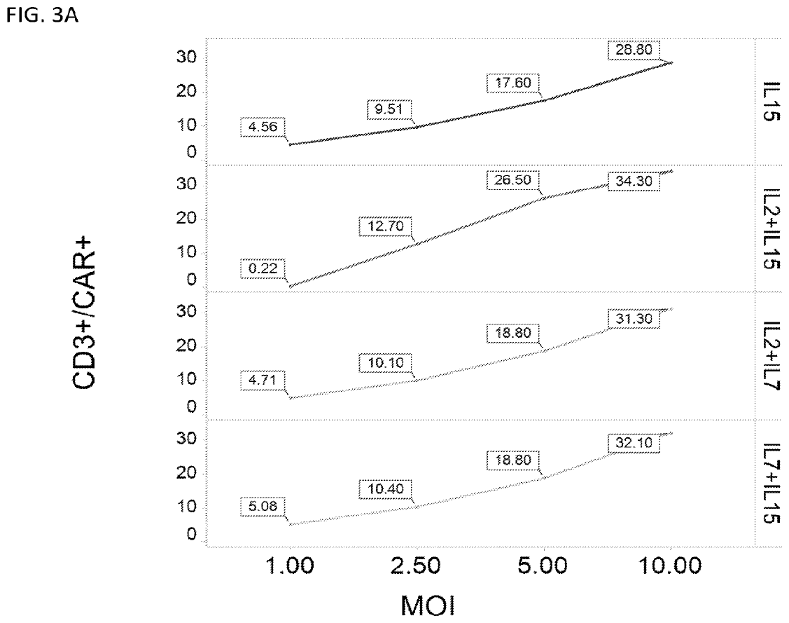

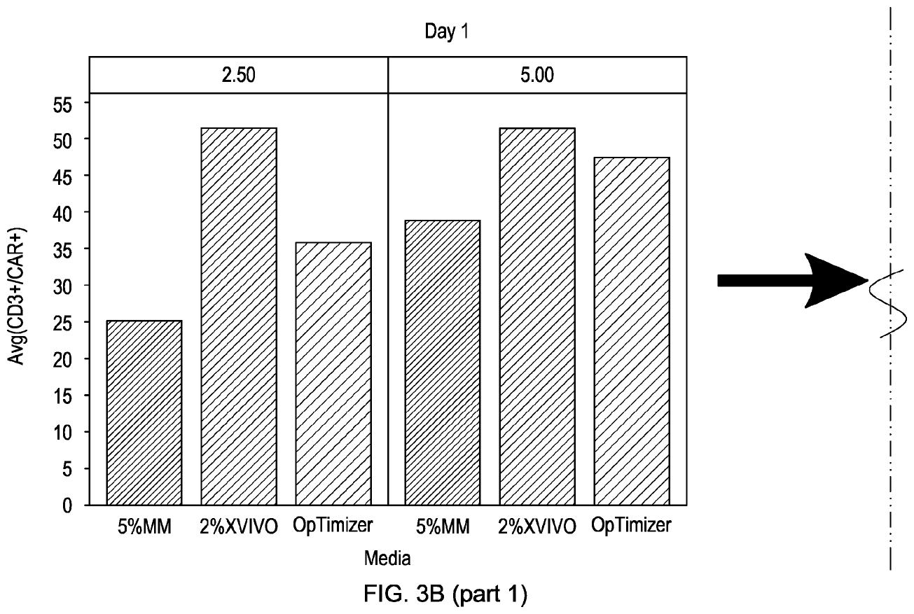

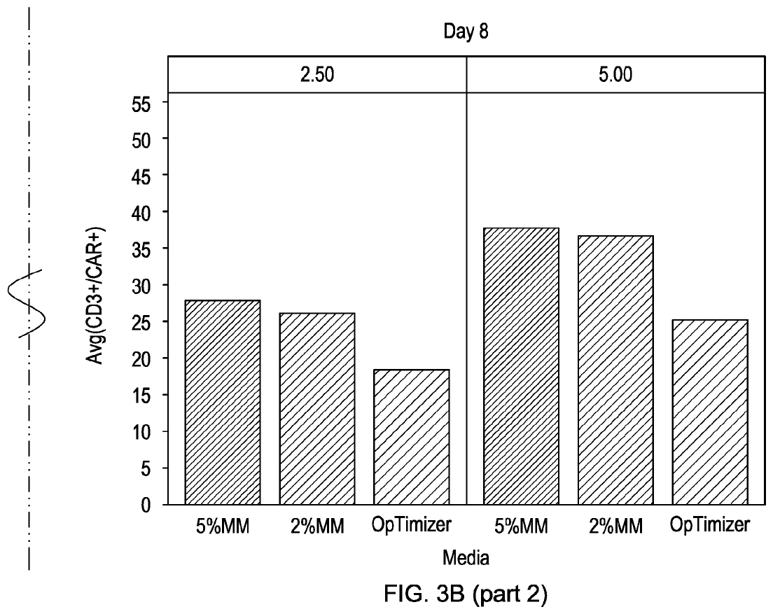

Although stable transduction was observed in the day 10 CARTs, the efficiency was consistently low. A titration of increasing multiplicity of infection (MOI) of the lentiviral vector was tested in four cytokine conditions and in all conditions tested a linear relationship with transduction was observed (FIG. 3A).

Furthermore, different media compositions (mainly a reduction in serum concentration from 5% to 2% to serum free) were compared to determine whether they impact the transduction efficiency. The reduction in serum to 2% human serum led to the highest transduction efficiency (FIG. 3B). The addition of Glutamax alone was also considered to have a significant impact on transduction efficiency.

Next, the day 1 CARTs and day 10 CARTs were examined for their anti-tumor activity in vivo using a mouse ALL model. Briefly, day 1 CARTs and day 10 CARTs were manufactured as described above with a viability above 80% (FIGS. 4A and 4B). CARTs were administered in tumor-bearing mice and monitored for expansion in vivo. As shown in FIG. 4C, day 1 CARTs showed a higher level of in vivo expansion than their day 10 counterparts. In particular, CARTs manufactured in the presence of IL-2 showed the highest level of in vivo expansion (FIG. 4C). All the CARTs tested inhibited tumor growth in vivo, although day 1 CARTs showed a delayed kinetics as compared to the day 10 CARTs (FIG. 4D). In this specific donor, the IL2 condition demonstrated the greatest ability to eliminate the tumor in vivo (FIG. 4D).

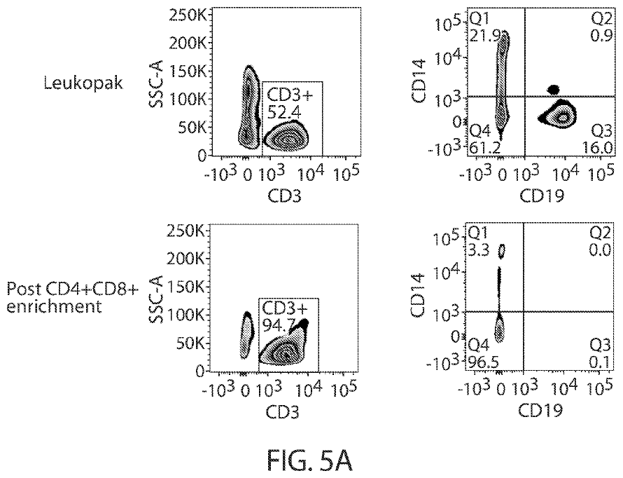

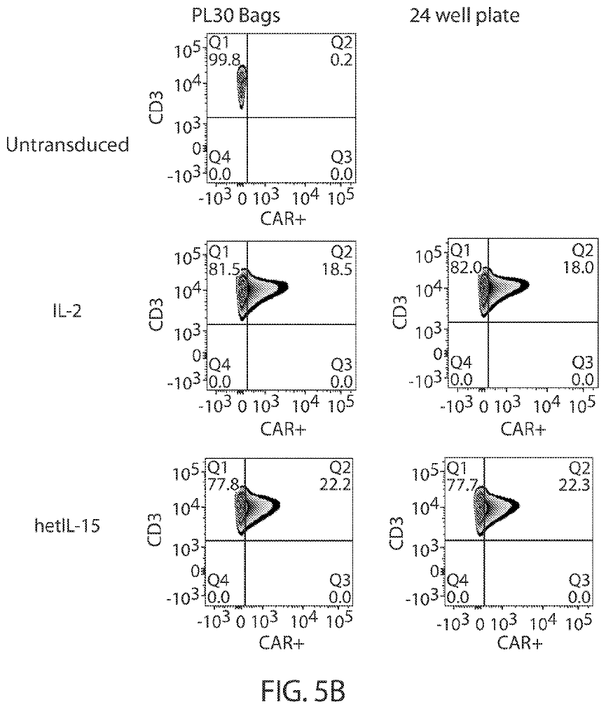

Furthermore, it was tested whether this manufacturing process was scalable. Purified T cells from a frozen apheresis sample were transduced with CAR19 in either a 24 well plate or a PL30 bag post enrichment, in the presence of either IL2 or hetIL-15 (IL15/sIL-15Ra). hetIL-15 has been described in WO 2014/066527, herein incorporated by reference in its entirety, and comprises human IL-15 complexed with a soluble form of human IL-15Ra. Cells were harvested 24 hours later and tested for expression of CAR. As shown in FIG. 5B, there was no impact on transduction observed when the process was scaled from a 24 well plate to a PL30 bag in the presence of either IL2 or hetIL-15.

### Example 2

### Generation of CARTs with TCR Stimulation

### Summary

This example describes a CART manufacturing process called “activation process.” In some embodiments, cells (for example, T cells) are seeded in media (for example, serum-free media, for example, OpTmizer™ media) containing IL-2 (for example, OpTmizer™ media containing OpTmizer™ supplement, GlutaMAX and 100 IU/ml of IL-2), placed in a cell culture device, and contacted with anti-CD3/anti-CD28 (for example, TransAct). After 12 hours, a vector (for example, a lentiviral vector) encoding a CAR is added to the cells and the cells are returned to an incubator. At 24 hours from initiation of the cell culture, the cells are harvested, sampled, and formulated. Without wishing to be bound by theory, brief CD3 and CD28 activation, for example, using anti-CD3/anti-CD28 (for example, TransAct), promotes efficient transduction of self-renewing T cells.

In this and other examples, a CART manufacturing process called “traditional manufacturing (TM)” process was used as a control. In some embodiments, T cells are selected from a fresh or cryopreserved leukapheresis sample (for example, using positive or negative selection), activated (for example, using anti-CD3/anti-CD28 antibody coated Dynabeads®), contacted with a nucleic acid molecule encoding a CAR molecule (for example, transduced with a lentiviral vector comprising a nucleic acid molecule encoding the CAR molecule), and expanded in vitro for, for example, 7, 8, 9, 10, or 11 days. An exemplary TM process is provided in this example as the methods used to manufacture CAR cells from the d9 control arms.

### Methods

In some embodiments, the activation process provided herein starts with a frozen or fresh leukapheresis product. After a sample for counting and QC is obtained, the product is attached to a cell sorting machine (for example, an installed CliniMACS® Prodigy® device kit) and the program begins. The cells are washed and incubated with microbeads that bind to desired surface marker or markers (such as CD3, CD4, CD8, CD27, CD28, CD45RO, CCR7, CD62L, CD14, CD34, CD95, CD19, CD20, CD22, and/or CD56). The bead-labeled cells are selected by passing the cells through a magnetic column. If desired, cells can be further separated by incubating the negative fraction with beads that bind to a second set of surface markers (such as CD3, CD4, CD8, CD27, CD28, CD45RO, CCR7, CD62L, CD14, CD34, CD95, CD19, CD20, CD22, and/or CD56) and again passing the cells through a magnetic separation column. Isolated cells are washed again and the separation buffer is exchanged for cell media. Purified cells then either proceed to culture or are cryopreserved for later use. Cryopreserved cells can be thawed, washed in pre-warmed cell media, and resuspended in cell media. Fresh cells can be added to culture directly. The cells are seeded into membrane bioreactors at 0.4-1.2e6 cells/cm² of membrane, an activating reagent such as anti-CD3/anti-CD28 beads/polymers, nanoparticles, or nanocolloids (and/or any of the following co-activators alone or in combination: a reagent that stimulates ICOS, CD27, HVEM, LIGHT, CD40, 4-1BB, OX40, DR3, GITR, CD30, TIM1, CD2, or CD226) is added, and cell media is added to a final volume of 0.25-2 ml/cm² of membrane. A vector (for example, a lentiviral vector) encoding the CAR is added immediately or up to 18 hours after culture initiation. The cells are incubated with the vector and the activating reagent described above for a total of 24 hours post culture initiation. Once culture has proceeded for 24 hours, the cells are resuspended mechanically by swirling or pipetting or otherwise agitating, and simulating reagent scaffolds are dissolved with appropriate buffers. The cells are washed to remove unnecessary reagents and reformulated in cryopreservation media. The cells are cryopreserved until needed for administration.

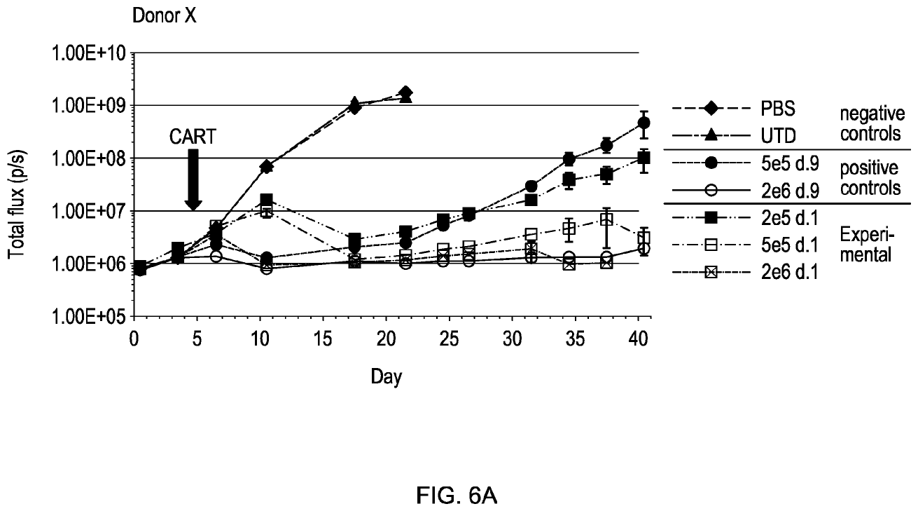

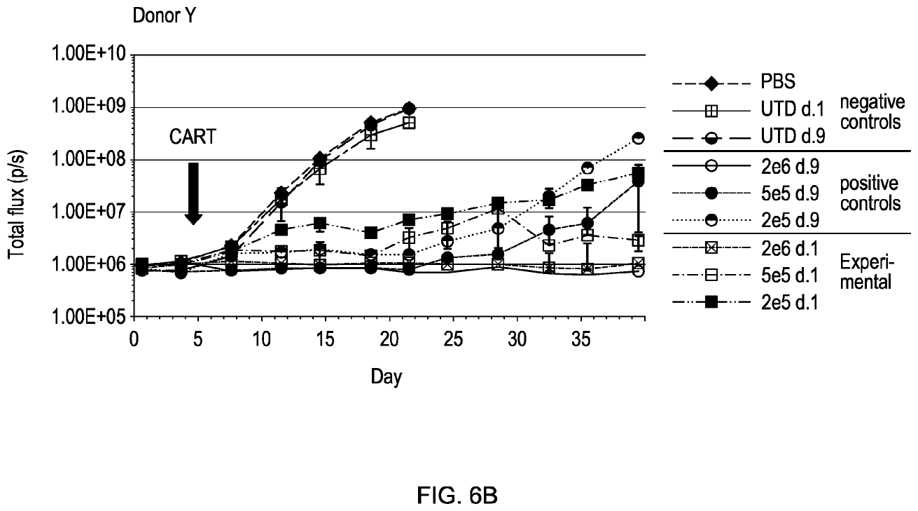

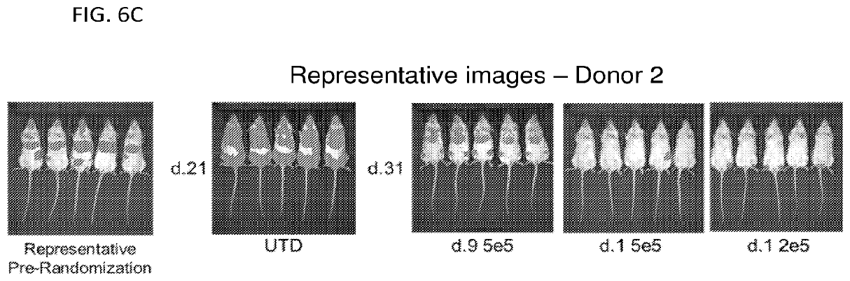

For studies related to FIGS. 6A-6C, the following protocol was used.

Cells were purified from a fresh ¼ leukopack using automated ficoll (Sepax 2, BioSafe) to generate peripheral blood mononuclear cells (PBMC). These PBMCs were further purified using immunomagnetic negative selection (PanT Negative Selection Kit, Miltenyi) to generate CD3 T-cells of high purity (98-100%). These cells were placed in culture with OpTmizer™ (Thermo) complete media (formulated per package insert and supplemented with IL-2 at 100IU/ml (Proleukin, Prometheus)) and an anti-CD3/CD28 activation reagent at the recommended dose (TransAct, Milenyi) in a membrane bioreactor. Cells were then incubated at 37° C., 5% CO₂ for 12 hours for activation. Cells were removed from the incubator and freshly thawed lentiviral vector was added to the cultures at a multiplicity of infection (MOI) of 2.5 tu/cell. Cells were returned to the incubator for another 12 hours for transduction. Cells were harvested, washed twice with media, and formulated directly into sterile PBS (Invitrogen) and injected into NSG mice via the tail vein. Cells from the d9 control arms were grown in flasks (T25-T225, Corning) using RPMI media (Thermo) supplemented with 10% fetal bovine serum (Seradigm) (complete media a.k.a “R10”) and anti CD3/28 Expander Dynabeads® (Thermo) at 3 beads per T-cell. Cells were then incubated at 37° C., 5% CO₂ for 24 hours for activation. Cells were removed from the incubator and freshly thawed lentiviral vector was added to the cultures at a MOI of 2.5 tu/cell. Cells were returned to the incubator for an additional 7 days, splitting every 2 days to maintain a concentration of 5e5 cells/ml. Expanded cells were transferred to 50 ml centrifuge tubes (Corning) and subjected to two rounds of bead removal using a standing magnet (Dynamag-50, Thermo). Debeaded cells were then washed twice with media, and formulated into CryoStor10 cryomedia (STEMCELL Technologies), cryopreserved using a CoolCell device (BioCision), and kept in vapor phase liquid nitrogen for a minimum of 48 hours. Cells were thawed into prewarmed R10 media, washed twice with media, then formulated into sterile PBS (Invitrogen) and injected into NSG mice via the tail vein.

6-8 week old NSG mice (NOD.Cg-PrkdcscidIl2rgtm1Wjl/SzJl, Jackson Labs) were injected with luciferized NALM6 tumor cells (ATCC CRL-3273, ATCC) at 1e6 cells/mouse 4 day prior to CART injection without preconditioning. PBS formulated CART cells were injected at 2e6, 5e5, or 2e5 CAR+ cells per NSG or a matched dose of untransduced expanded T-cells or a PBS vehicle control. Mice were monitored by weekly blood draw, bi-weekly luciferase imaging (Xenogen IVIS, PerkinElmer), and bi-weekly weight measurements. All animals were monitored for signs of toxicity (weight loss, moribund) and euthanized if symptomatic. All surviving mice were euthanized at study termination (week 5) and terminal blood, bone marrow, and spleen samples were obtained. Study was performed according to IACUC and all other applicable guidelines.

### Results

CART cells were generated using the activation process described above and characterized for their in vivo anti-tumor activity in a mouse ALL model. As shown in FIGS. 6A-6C, CART cells manufactured using the activation process showed strong anti-tumor activity in vivo.

### Example 3

### IL6R Expression on T Cells and Cytokine Effect on T Cell Expansion

### Material and Methods

### T Cell Culture

Previously frozen T cells were thawed and contacted with αCD3/αCD28 dynal beads (cell to bead ratio of 1 to 3) in the presence of indicated cytokines at day 0. From day 3, twice more T cell growth media (RPMI1640, 10% FBS, 2 mM L-glutamin, 100 μM non-essential amino acids, 1 mM sodium pyruvate, 10 mM Hepes, 55 μM β-mercaptoethanol, 10% FBS, and 100 U/ml of penicillin-streptomycin) was added to the plate with indicated cytokines (without cytokine, rhIL2 (50 IU/ml, Novartis), IL6 (long/ml, R&D systems), IL7 (long/ml, Peprotech), IL15 (long/ml, Peprotech), and IL21 (long/ml, Peprotech)) at day 3, 5, 6, 9, 12, 15, and 18. Cells treated without cytokine, IL6, or IL21 were cultured until day 18 and cells treated with IL2, IL7, or IL15 were cultured until day 25.

### Cell Surface Staining

Cells were harvested at indicated time points and then stained with live/dead dye (eFluro780, eBioscience), CD3 (BioLegend, clone#: OKT3), CD4 (BioLegend, clone#: OKT4), CD8 (BD Bioscience, clone#: RPA-T8), CD45RO (BioLegend, clone#: UCHL1), CCR7 (BioLegend, clone#: G043H7), CD27 (BD Horizon, clone#: L128), CD127 (BioLegend, clone#: A019D5), CD57 (BioLegend, clone#: HCD57), CD126 (BioLegend, clon#: UV4), and CD130 (R&D Systems, clone#: 28126) antibodies. The cells were acquired by FACS Fortessa and then FlowJo program was used for data analysis.

### Intracellular Cytokine Staining

To examine percent of cytokine producing cells, at day 25, T cells were harvested and then briefly activated with PMA (50 ng/ml, Sigma-Aldrich) and lonomycin (1 82 M, Sigma-Aldrich) for 4 hours in the presence of Brefeldin A (BioLegend) at 37° C. incubator. T cells were then stained with live/dead dye (eFluro780, eBioscience), CD3 (BioLegend, clone#: OKT3), CD4 (BioLegend, clone#: OKT4), CD8 (BD Bioscience, clone#: RPA-T8) antibodies followed by fixation and permeabilization. Then, T cells were further stained with antibodies against IFN-γ (BioLegend, clone#: 4S.B3), IL-2 (BioLegend, MQ1-17H12), and TNF-a (BioLegend, Mab11). The cells were acquired by FACS Fortessa and then FlowJo program was used for data analysis.

### Results

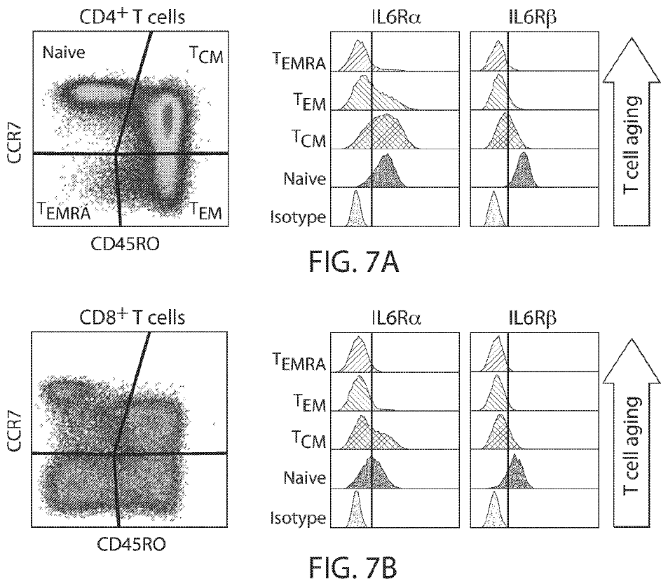

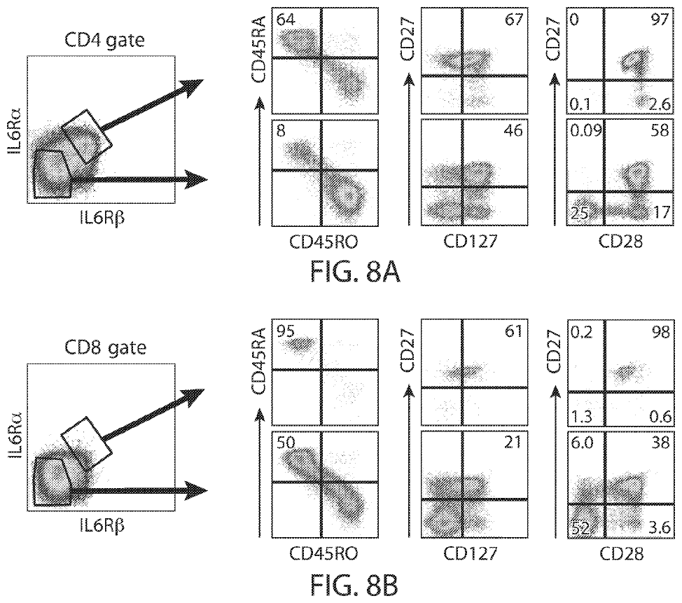

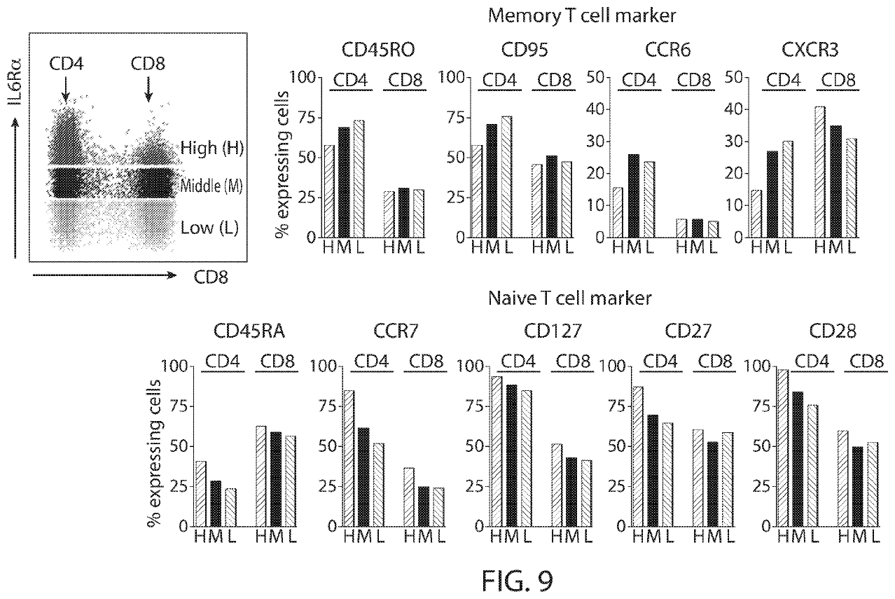

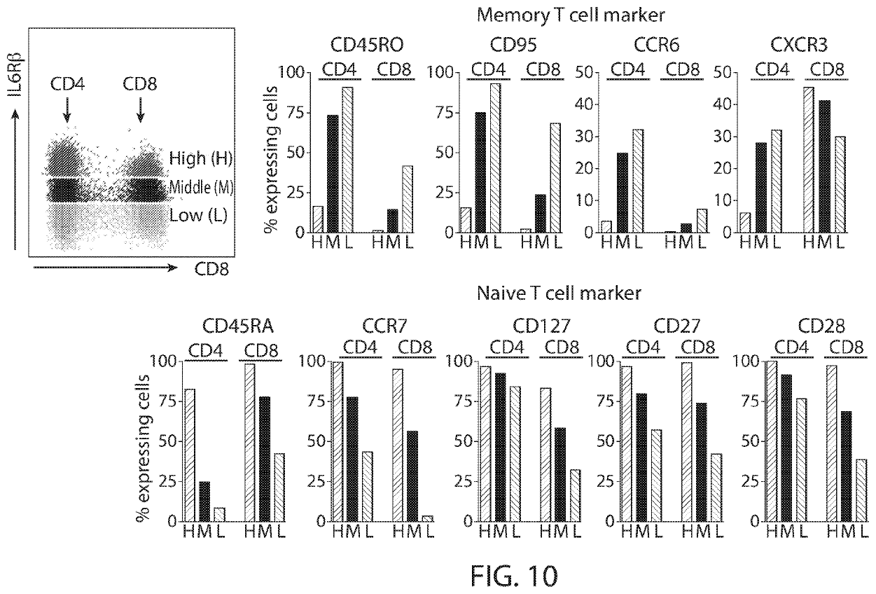

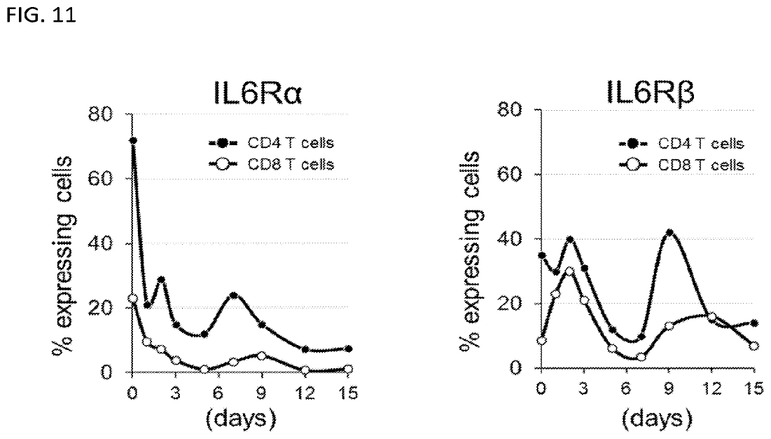

IL6Rα and/or IL6Rβ expressing cells were enriched in less differentiated T cell subsets in both CD4 and CD8 T cells. As shown in FIGS. 7A and 7B, naïve CD4 and CD8 T cells expressed higher levels of IL6Rα and IL6Rβ than the corresponding memory T cells. T cells that expressed both IL6Rα and IL6Rβ were predominantly CD45RA+CD45RO−CD27+CD28+ cells (FIGS. 8A and 8B). Upon TCR stimulation, IL6Rα but not IL6Rβ expression was down-regulated (FIG. 11).

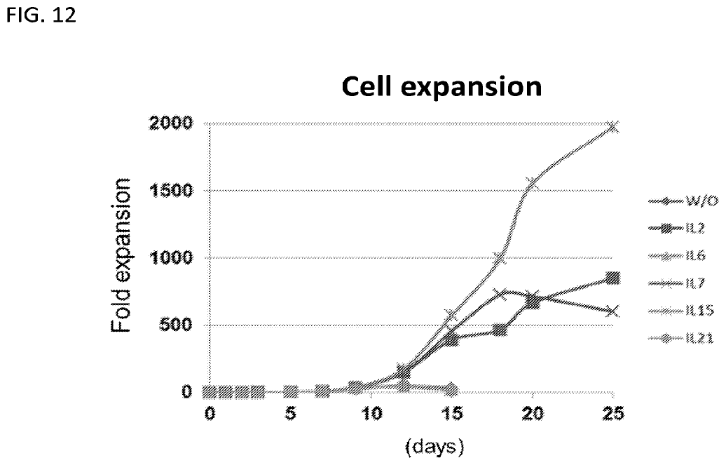

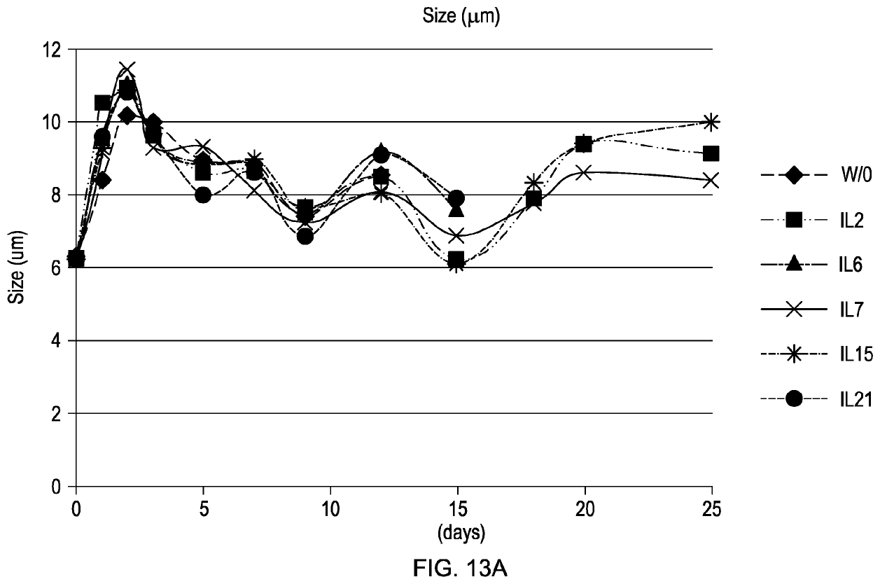

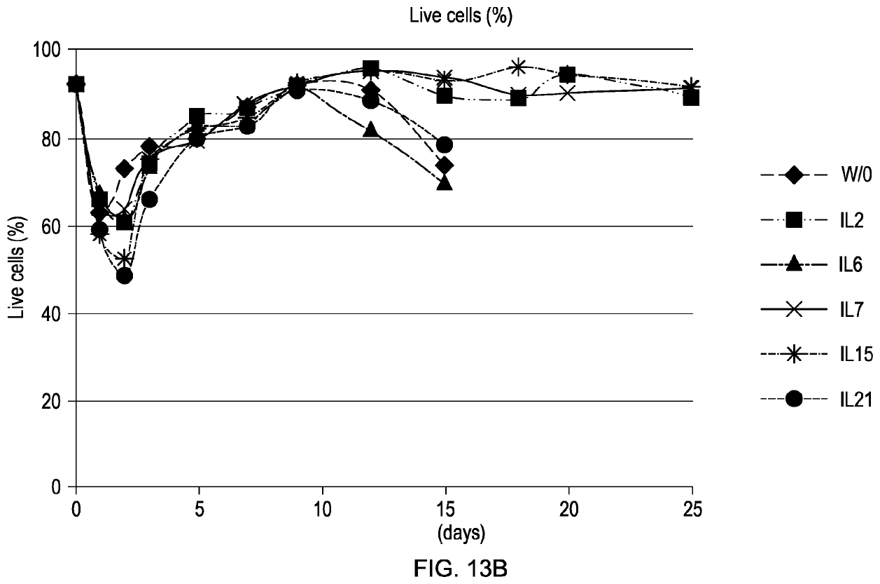

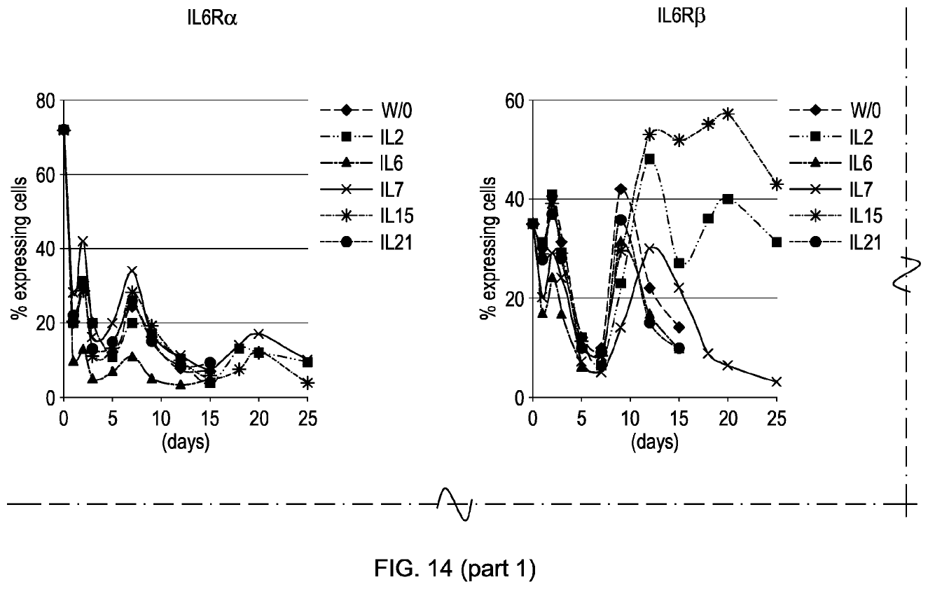

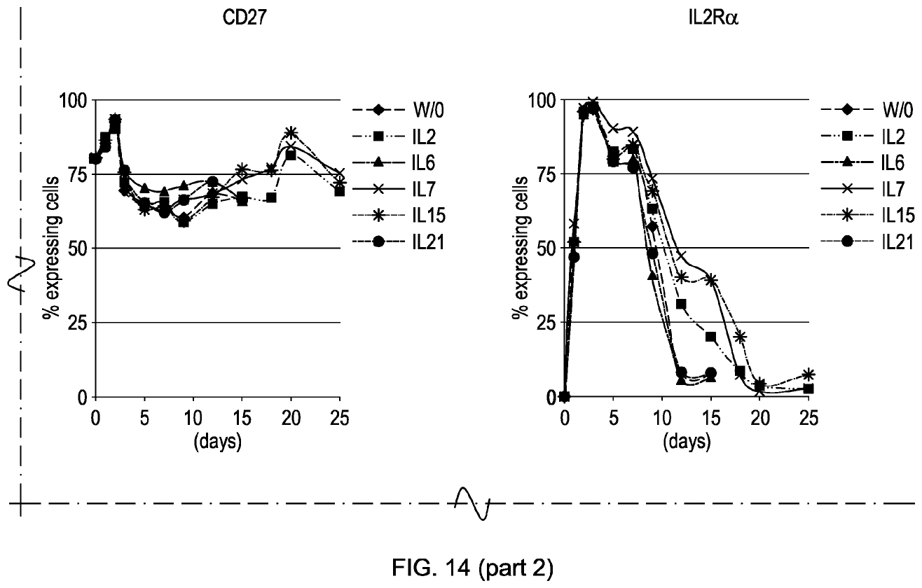

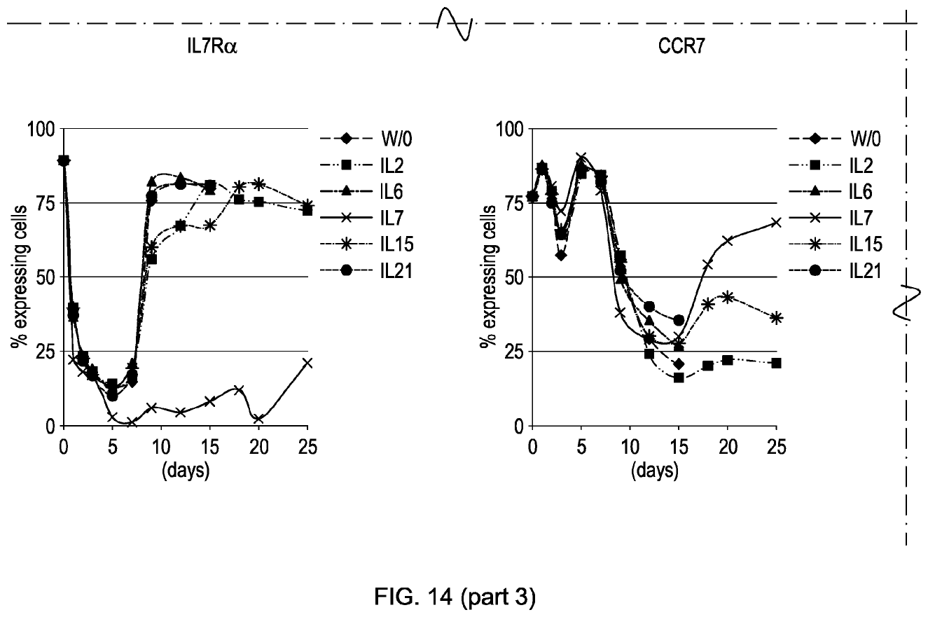

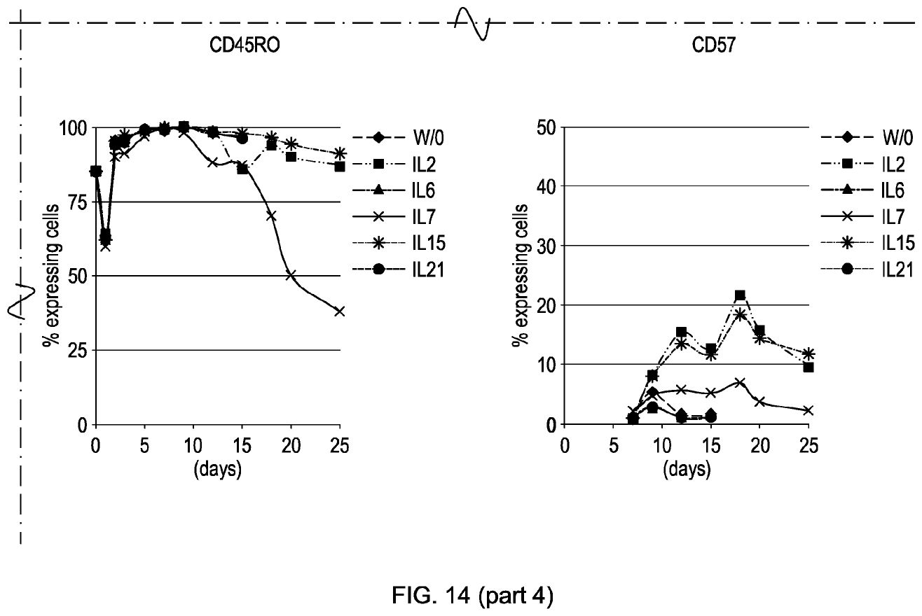

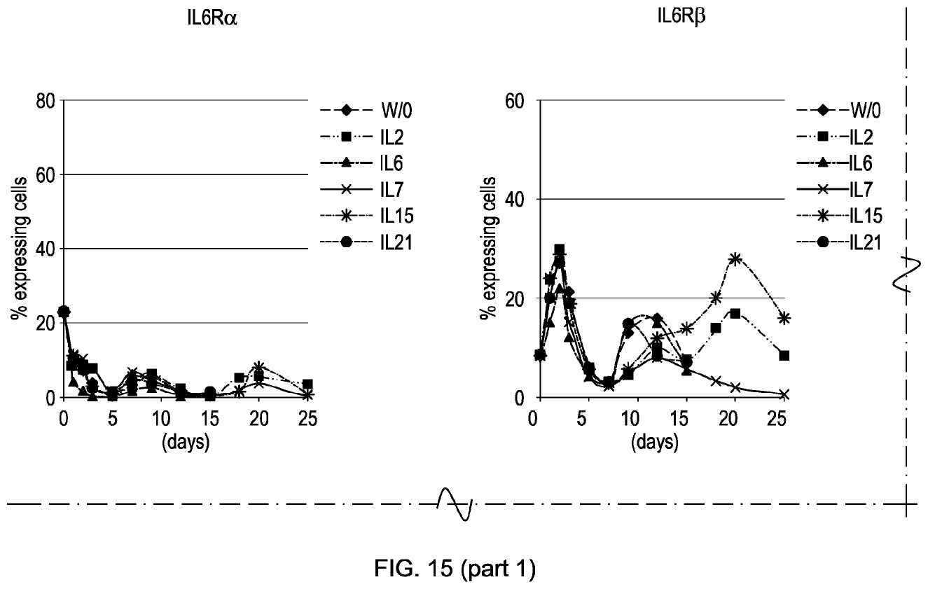

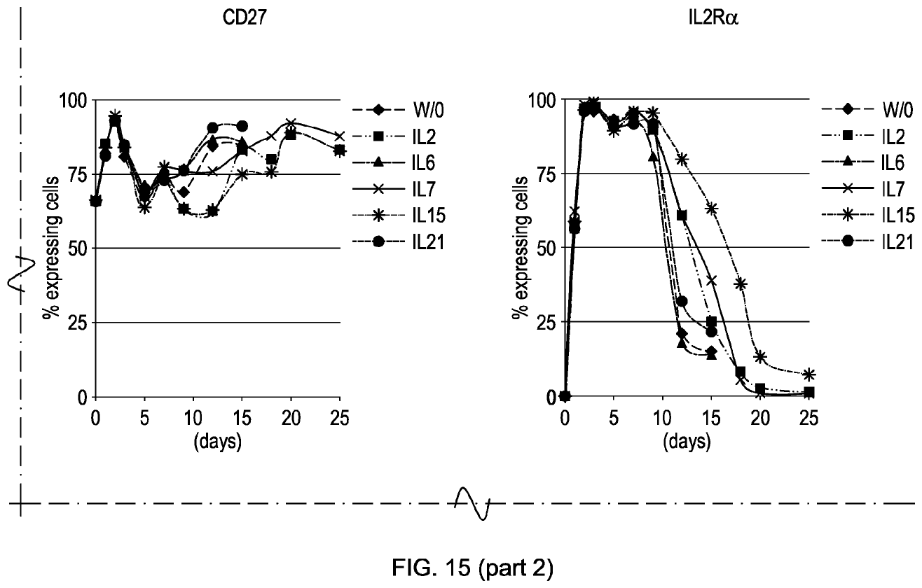

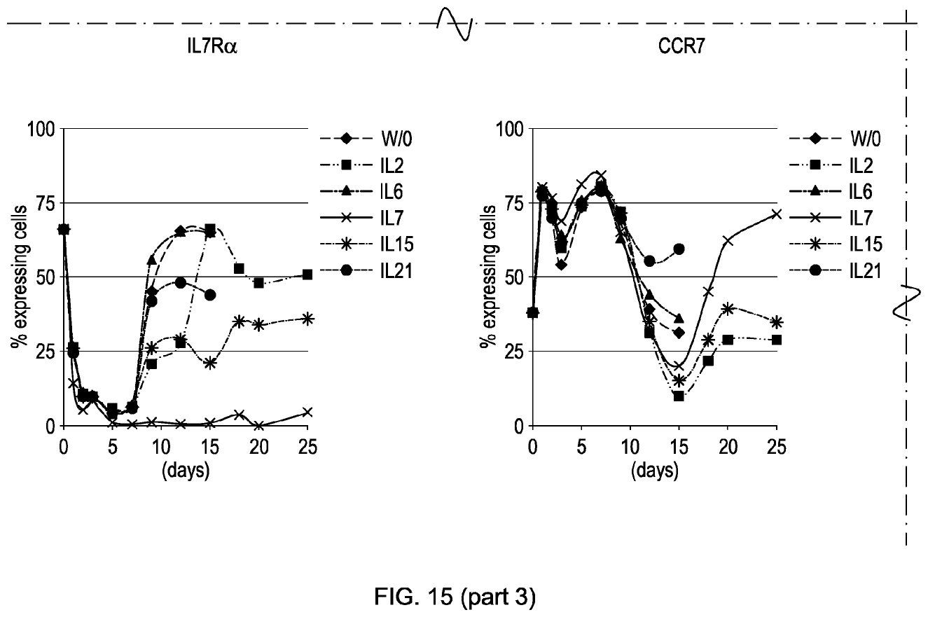

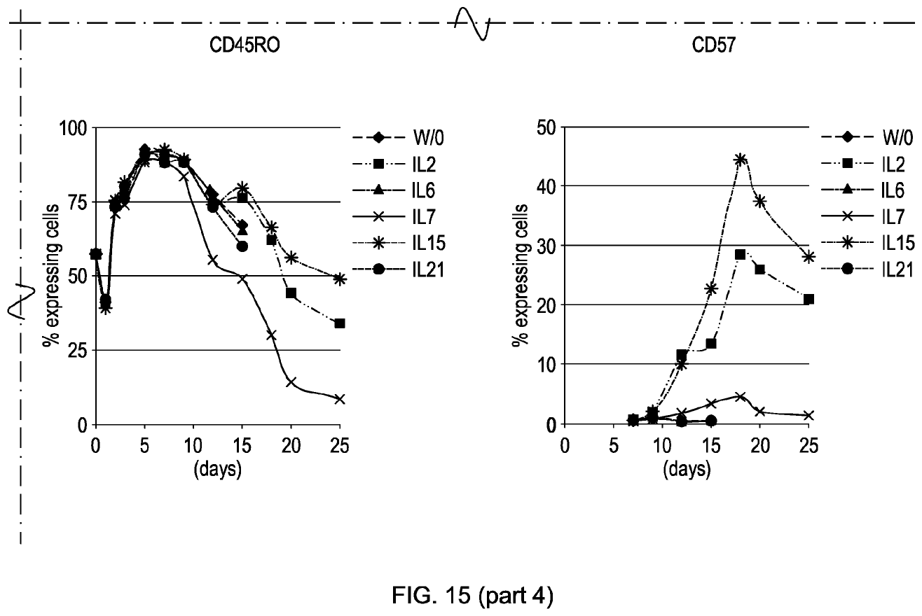

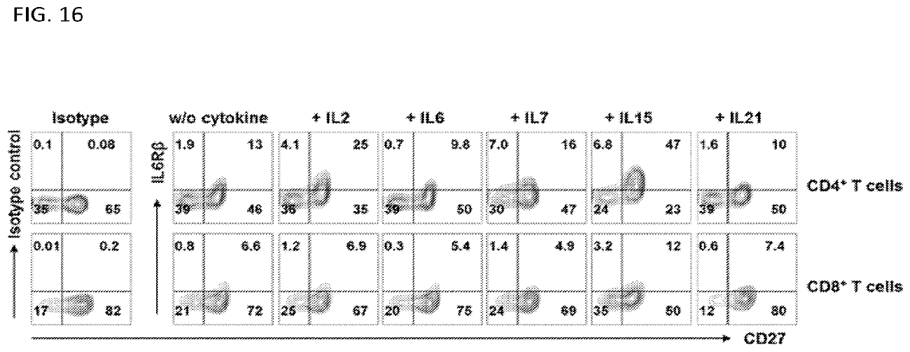

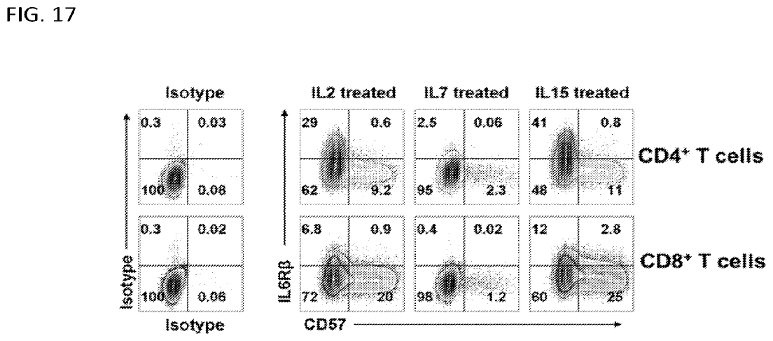

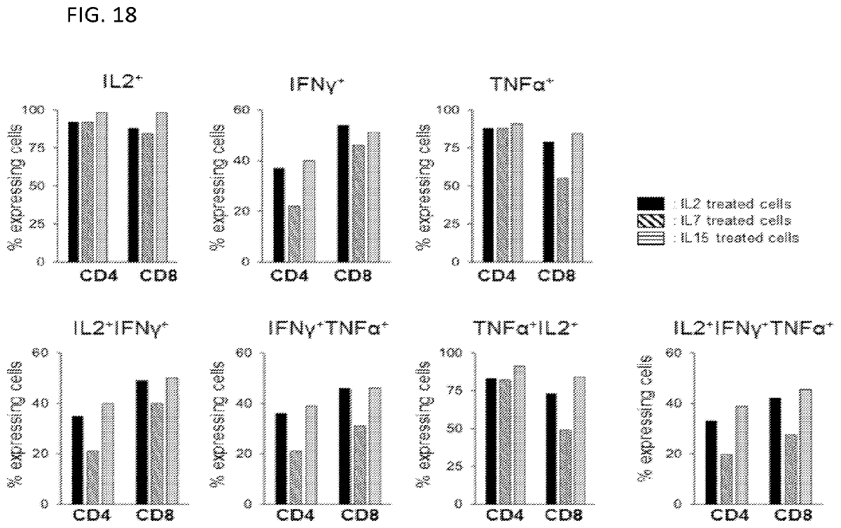

Next, different cytokines were compared for their impact on T cell expansion. Among the cytokines tested, IL15, IL2, and IL7 enhanced T cell expansion, with IL15 showing the greatest enhancement (FIG. 12). Cytokine treatment did not affect cell size (FIG. 13A) or viability (FIG. 13B). IL15 treatment also enhanced expansion of IL6Rβ expressing cells (FIG. 14). IL6Rβ expressing cells were mainly in the CD27+ (FIG. 16) or CD57− (FIG. 17) T cell subsets in both CD4 and CD8 at day 15 after TCR engagement and produced IL2, IFNγ, and TNFα cytokines at day 25 after TCR activation (FIG. 18).

### Example 4

### Generation of CARTs with TCR Stimulation for Preclinical Studies

Day 0 unit operations of the engineering runs for preclinical studies began with the manufacturing of the media used on Day 0: Rapid Buffer and Rapid Media (Table 21). The Rapid Buffer (RB) contains the CliniMACS® buffer (Miltenyi) with 0.5% HSA. The Rapid Media (Table 21) was formulated on Day 0 of manufacturing and the base media contains the off-the-shelf media called OpTmizer™ which has Glutamax, IL-2, CTS™ supplement, and ICSR . The Prodigy® machine was primed for use on Day 0.

TABLE 21
Media type and point of use during CART manufacturing

| Media/Buffer | | |
|---|---|---|
| Type | Composition | Point of Use |
| Rapid Buffer (RB) | CliniMACS ® Buffer | Day 0 Processing on Cell |
| | (+0.5% human serum | Wash/Separator |
| | albumin (HSA)) | |
| Rapid Media (RM) | OpTmizer ™ Media, | Day 0 for Processing on |
| | CTS ™, IL-2, | Cell Wash/Separator and |
| | Glutamax and ICSR | Cell Seeding |
| Harvest Buffer | PBS no EDTA and 2% | Harvest Wash Buffer (Day |
| (HB) (also called | HSA | 1) |
| Harvest Buffer | | |
| Solution) | | |
| Cryomedia | Cryostor10 (CS10) | Harvest Formulation |

As the Prodigy® machine was priming on Day 0, the healthy donor leukapheresis material was thawed and the apheresis material was combined into a 600-mL transfer bag that can later be welded onto the Prodigy®. An IPC sample was extracted from the 600 mL transfer bag and measured by NC200 to obtain both the viable cell count and the viability percentage for the starting apheresis material. After priming of the Prodigy® was finished, the apheresis material was transferred to the application bag. After the apheresis entered the Prodigy® machine after initiation of the TCT program, the program ran from 3 h 45 min to 4 h 15 min depending on how many positive selection separations it performed. The TCT program on Day 0 washed out the DMSO in the Centricult with the Rapid Buffer, performed a platelet wash, volume reduction, incubation of the apheresis with the CD4 and CD8 Microbeads in the Centricult, and then selection of the T cells with the Microbeads via positive selection using the magnet on the Prodigy®. The T cells selected with the CD4 and CD8 reagents were eluted into the reapplication bag with the Rapid Media. An in-process control (IPC) sample was taken from the reapplication bag to determine the total viable cell number available for seeding in the culture vessel (G-Rex500MCS).

The G-Rex culture device was first primed with the Rapid Media and then the target cell volume from the reapplication bag was added to the culture vessel. The activation reagent (TransACT) was then added to the culture vessel. The lentiviral vector was then added to the culture vessel after the introduction of TransACT and the vector addition was performed using a MOI of 1.0. The G-Rex500MCS culture vessel was then flushed with the Rapid Media to a final media volume of 250 mL plus the volume of the vector addition. The G-Rex culture vessel was then placed into the incubator to allow the culture to incubate for a target 24 h with a range of 20-28 hours.

After the target 24 h incubation, the CART culture was taken out of the incubator and a sample was extracted to obtain the viable cell count and viability of the cell culture before the Harvest Wash. The sample take at Pre-Harvest was an IPC and was used as an input into the LOVO wash device to determine the flow rate of cells into the spinning filtration membrane. The LOVO used the viable WBC concentration as the IPC. The program used for the CART manufacturing process was described as 4 Washes with one solution and utilized the Harvest Buffer (PBS+2.0% HSA). During the LOVO wash, the IPC bag was used to both reduce the volume and wash the cells with Harvest Buffer before it was finally eluted into the output bag. The output bag from the LOVO wash was then sampled to obtain the viable cell count and viability in order to perform the manual centrifugation with the sanisure bottle and to perform the final steps of the final formulation with the cryomedia.

### Example 5

### Generation of BCMA CARTs Using the Activated Rapid Manufacturing (ARM) Process

### Summary

This example describes a CART manufacturing process called “activated rapid manufacturing (ARM).” In some embodiments, cells (for example, T cells) are cultured in a cell culture device containing media (for example, serum-free media, for example, OpTmizer™ media), recombinant human IL-2 (for example, OpTmizer™ media containing OpTmizer™ supplement, GlutaMAX and 100 IU/ml of IL-2), anti-CD3/anti-CD28 (for example, TransAct) and a vector (for example, a lentiviral vector) encoding a BCMA CAR. After 24 hours, the cells, referred as “day 1 CART product” are harvested, sampled, and formulated. Without wishing to be bound by theory, brief CD3 and CD28 activation, for example, using anti-CD3/anti-CD28 (for example, TransAct), promotes efficient transduction of self-renewing T cells. In some cases, some cells are harvested at 48 h, 72 h, and 96 h or 7 days after culture for measuring BCMA CAR expression kinetics in vitro. The day 1 CART responses include, but are not limited to, in vivo cytolytic activity and expansion.

### Generation of Day 1 BCMA CARTs Using the ARM Process

In some embodiments, the activation process provided herein starts with a frozen or fresh leukapheresis product. After a sample for counting and QC is obtained, the product is attached to a cell sorting machine (for example, an installed CliniMACS® Prodigy® device kit) and the program begins. The cells are washed and incubated with microbeads that bind to desired surface markers, such as CD4 and CD8. The bead-labeled cells are selected by passing the cells through a magnetic column. Isolated cells are washed again and the separation buffer is exchanged for cell media. Purified T cells then either proceed to culture or are cryopreserved for later use. Purity of the isolated T cells will pass a QC step by flow cytometry assessment. Cryopreserved cells can be thawed, washed in pre-warmed cell media, and resuspended in cell media. Fresh cells can be added to culture directly. The cells are seeded into membrane bioreactors at 0.4-1.2e⁶ cells/cm² of membrane, an activating reagent, such as anti-CD3/anti-CD28 beads/polymers, nanoparticles, or nanocolloids, is added, and cell media is added to a final volume of 0.25-2ml/cm² of membrane. At the time of plating, the cells are transduced with a lentiviral vector encoding BCMA CAR at various multiplicity of infections (MOIs). The titer and the MOI are measured based on cell lines such as SupT1. At 24 hours, the cells are washed to remove unnecessary reagents before staining to measure the CAR expression by flow cytometry and reformulated in cryopreservation media as “day 1 CART product” for in vivo study.

Described in this example are the generation and characterization of T cells expressing BCMA CAR R1B6, R1F2, R1G5, PI61, B61-02, B61-10, or Hy03, manufactured using the ARM process. The sequences of R1B6, R1F2, and R1G5 are disclosed in Tables 3-6. The sequences of PI61, B61-02, and B61-10 are disclosed in Tables 7-11. The sequences of Hy03 are disclosed in Tables 12-15.

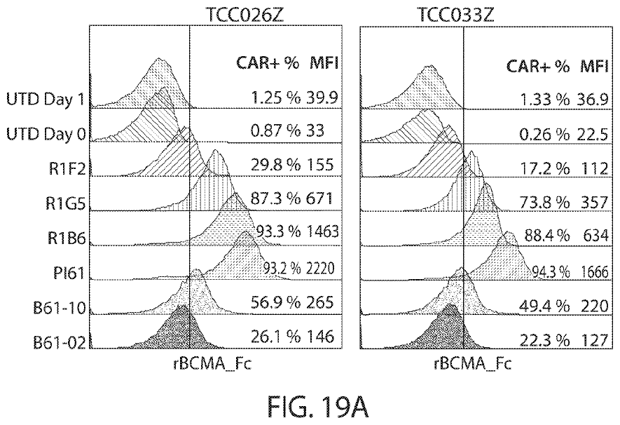

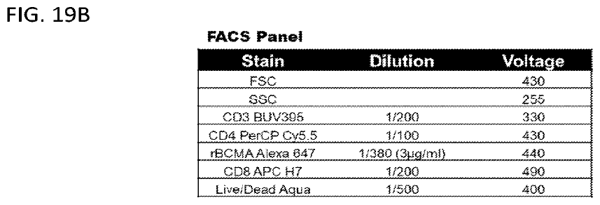

Twenty-four hours after T cells were transduced using lentiviral vectors encoding BCMA CARs at a MOI of 2.5, the expression of CAR was measured by flow cytometry using rBCMA_Fc. As shown in FIG. 19A, it was observed that the whole population of the live CD3+ T cells shifted to the right at different degrees. Cells transduced to express R1G5, R1B6 or PI61 showed the highest CAR expression (FIG. 19A). This pattern of expression as measured by flow cytometry was different from a typical flow cytometry histogram of cells transduced to express a CAR, where a CAR positive population is clearly separated from a negative population. FIG. 19A indicates that there may be “pseudotransduction or transient expression” detected by rBCMA_Fc, which does not always indicate real gene expression. It has been previously reported that lentiviral pseudotransduction was observed beginning at the time of vector addition and lasting up to 24 hours in CD34+ cells and up to 72 hours in 293 cells (Haas D L, et al. Mol Ther. 2000. 291: 71-80). Integrase-defective lentiviral vector caused transient eGFP expression for up to 10 days in CD34+ cells and for up to 14 days in 293 cells. Though lentiviral pseudotransduction has not been extensively studied in T cells, this possibility of transient expression in such a short time cannot be ruled out. Therefore, in vitro kinetic study was performed to measure CAR expression of cells manufactured using ARM as indicated below.

### In Vitro CAR Expression Kinetics Study of Cells Manufactured Using the ARM Process

The study described here examines how cells manufactured using the ARM process express CAR molecules over time. Briefly, T cells from a healthy donor were manufactured to express a BCMA CAR using the ARM process at a MOI of 1 and were kept in culture for different time periods and harvested at 24 h, 48 h, 72 h, 96 h, and day 7 for assessing CAR expression kinetics by flow cytometry using AF647 labeled rBCMA_Fc. Understanding the CAR expression kinetics helps to find a surrogate time point for real and stable expression for in vivo triage or clinical dosing strategy.

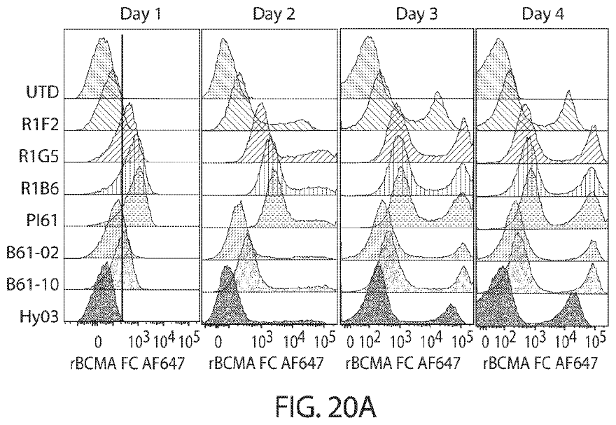

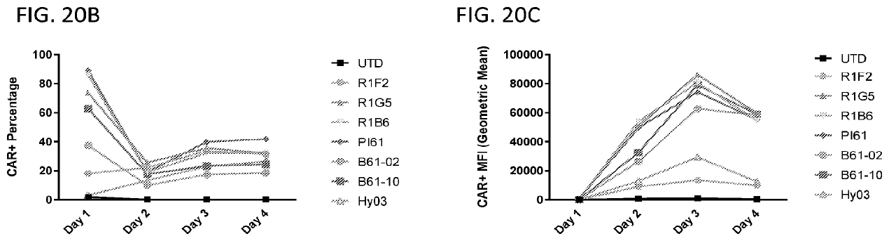

At day 1, the CAR expression pattern of cells transduced at a MOI of 1 (FIG. 20A) is similar to that of cells transduced at a MOI of 2.5 (FIG. 19A). Both MOI conditions showed a pseudo or transient expression pattern at day 1 (FIGS. 19A and 20A). However, at day 2, a rBCMA_Fc positive population started to be separated from the UTD negative control group (FIG. 20A). At day 3 and day 4, a rBCMA_Fc positive population, which represents the BCMA CAR-expressing cells and is absent in the UTD group, clearly showed up in all the groups where cells were transduced to express a BCMA CAR. From day 3 to day 4, the CAR+% was relatively stable for each CAR construct (FIG. 20B), with the highest MFI observed at day 3 (FIG. 20C) (the cells were the largest at this time point). Consistent with the data shown in FIG. 19A, cells transduced to express PI61, R1G5 and R1B6 were the highest CAR expressers (FIG. 20A). Notably, cells transduced with vectors encoding R1F2 or Hy03 did not show transient CAR expression at day 1 but clearly expressed BCMA CAR molecules later at day 3 and day 4 (FIG. 20A). In conclusion, vectors encoding different CARs may have different CAR expression kinetics over time, and day 3 was chosen as a surrogate time point for CAR expression.

### Evaluating Functionality of the Day 1 ARM Processed BCMA CART In Vivo

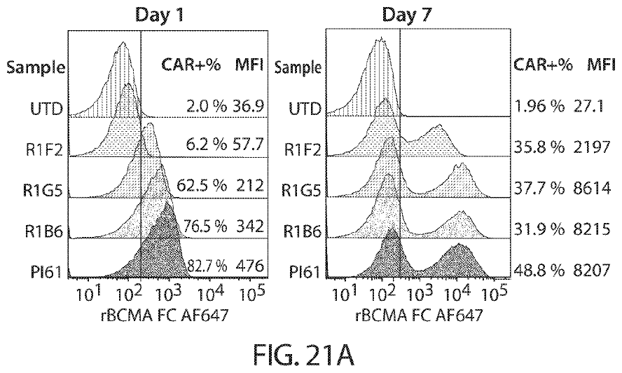

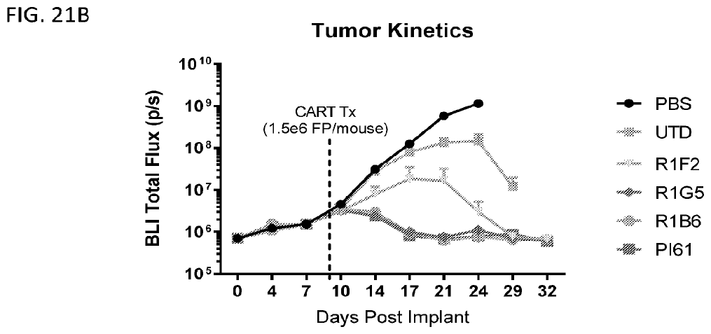

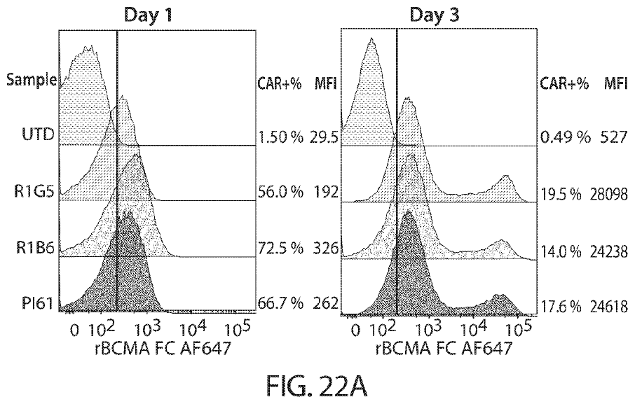

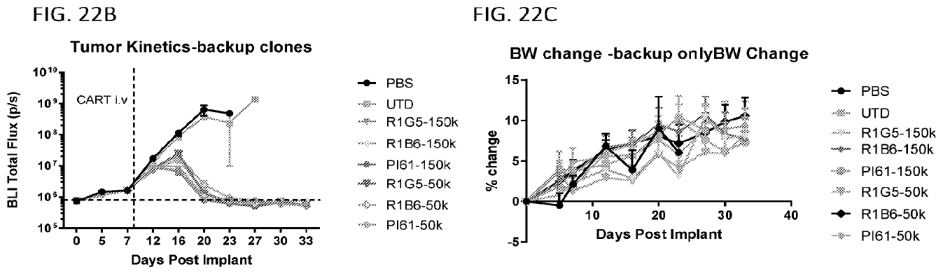

The day 1 CARTs were examined for their anti-tumor activity in vivo using a disseminated KMS-11-luc multiple myeloma xenograft mouse model. The luciferase reporter allows for monitoring of disease burden by quantitative bioluminescence imaging (BLI). Briefly, day 1 CARTs manufactured as described above were administered in tumor-bearing mice. In the first in vivo study (FIGS. 21A and 21B), each mouse received a final CART product at a dose of 1.5E6 cells. CAR expression was analyzed at day 1 and day 7 (FIG. 21A). In the in vivo efficacy study, cells expressing PI61, R1G5 or R1B6 demonstrated potent anti-tumor activities (FIG. 21B). Cells expressing R1F2 showed a delayed efficacy (FIG. 21B). The UTD group also showed partial anti-tumor activity 14 days after CART injection, which could be due to alloreaction (FIG. 21B). A second in vivo study tested dose titration of the CAR+T cells. The doses of CAR+T cells were based on CAR+ % at day 3 (FIG. 22A). Tumor intake kinetics was monitored twice a week by BLI measurement. FIG. 22A shows CAR expression detected at day 1 and day 3. The in vivo results indicate that all three clones PI61, R1B6 and R1G5 at both doses of 1.5e5 CAR+ T cells and 5e4 CAR+ T cells were able to reject and clear tumor as shown in FIG. 22B. FIG. 22C shows body weight changes over the course of this study, displaying no indication of GVHD.

### Example 6

### Kinetics of Rapid CARTs Harvested Between 12-24 Hours

### Introduction

To determine whether a rapid CART product could be generated in less than 24 hours, the kinetics for harvesting rapid CARTs generated after 12-24 hours in culture was characterized. This evaluation was performed at small scale using T cells enriched from cryopreserved healthy donor apheresis and simultaneous addition of TransAct activation reagent and technical grade CTL019 vector at seeding. Primary readouts were viability, viable cell recovery post-expansion, leukocyte and T cell subset composition, and transduction efficiency (as determined via surface immunophenotyping) on freshly harvested CART products.

### Methods

Lentivirus production and titer determination: The lentiviral vector encoding CTL019 was prepared with a HEK293T-based qPCR titer of 4.7×107 TU/mL and an approximated T cell-based titer of 1.88×107 TU/mL.

T cell isolation: A cryopreserved leukopak (LKPK) of healthy donor apheresis was obtained from Hemacare and stored in liquid nitrogen until needed. On Day 0, the apheresis was thawed until a small ice crystal remained, and then diluted with Prodigy® process buffer. Automated CD4/CD8 positive selection was then performed on the CliniMACS® Prodigy® with the TS 520 tubing set and T Cell Transduction (TCT) program software version 1.0. The final Prodigy® product was eluted in OpTmizer™ complete T cell medium, and cell concentration and viability were determined by AO/PI staining as enumerated by the Cellometer Vision (Nexcelom).

Culture initiation and transduction: Cells from the Prodigy product were immediately seeded into a total of seven vessels: five vessels for transduced cultures and two vessels for untransduced (UTD) cultures. At timepoint zero, each vessel was seeded at a density of 0.6×10⁶ viable cells per cm² of membrane, plus GMP-grade TransAct, and brought to a final concentration of 1.2 ×10⁶ viable cells/mL with OpTmizer™ complete T cell media containing IL-2. Vector was thawed at room temperature and added to each transduced culture at a MOI of 0.45 based on the approximated T cell titer. No virus was added to the UTD controls. Once seeded, cultures were incubated at 37° C. and 5% CO₂ until ready for harvest.

Harvest: At each timepoint 12 to 24 hours after culture initiation, one transduced culture was selected for harvest. Cells were harvested by swirling the vessel to gently resuspend the cells off the membrane, then the full culture volume resuspended and transferred by serological pipette to a conical tube. A small aliquot was taken for a pre-wash count, viability determination, and flow staining. The remainder of each culture was washed twice in 50 mL (twice in 100 mL for UTD vessels), resuspended, and a post-wash aliquot taken to examine counts and viability.

Flow cytometry of leukocyte composition and CD19-CAR expression during CART manufacturing: In-process samples before and after culturing were stained for leukocyte composition, T cell phenotype, and CAR expression where applicable. CTL019-CAR expression on transduced T cells was evaluated using a custom-ordered fluorophore-labeled anti-idiotype antibody (eBioscience). At each harvest timepoint, aliquots of the culture were immediately stained with viability dye (Biolegend), washed, then stained with two flow panels both containing a CD3 stain and the anti-idiotype antibody and fixed in paraformaldehyde for acquisition. Samples were measured on a flow cytometer (BD LSRFortessa; single color controls were used for compensation), and data was analyzed with FlowJo software. For analysis, all samples stained for leukocyte composition were pre-gated on viable CD45+ singlet events and all samples stained for T cell subsets were pre-gated on viable CD3+ singlet events. Gates for CD45RO and CCR7 were established using fluorescence minus one (FMO) controls.

### Results

The leukocyte composition of the LKPK, Prodigy® product before culture, and the CART products after culture were characterized using flow cytometry on Day 0 and each harvest time point. The cell types identified were T cells (CD3+), monocytes (CD14+), B cells (CD19+), natural killer (NK) cells (CD3−56+), and other cells (Table 22). Prodigy® enrichment produced a Day 0 starting material that was highly viable (92.9%) and enriched for T cells (from 48% to 92%) while reducing contaminating B cells (6% to 0.10%) and monocytes and NK cells to under 4% each. After 12-24 hours in culture, the purity of the viable cells increased an additional 3-4.4%, corresponding with an immediate reduction of monocytes and B cells by hour 12 and gradual reduction of NK cells between hours 12 and 24. Of the leukocytes that express extracellular CAR by flow cytometry, less than 3% were contaminant cells (i.e. not T cells), with the greatest jump in CAR purity (96.6% to 99.2%) occurring between 15 and 18 hours after seeding.

TABLE 22
Gross leukocyte composition of CART products
% of population

| | Product or | | | | CD3- | |
|---|---|---|---|---|---|---|
| Timepoint | Subpopulation | CD3+ | CD14+ | CD19+ | CD56+ | Other |
| Day 0 | LKPK | 48% | 29% | 6.0% | 11.6% | 5.0% |
| | Prodigy ® | 92% | 3% | 0.10% | 3.7% | 0.4% |
| | Product | | | | | |
| CARTs | 12 hr | 95.3% | 0.2% | 0.02% | 3.3% | 1.1% |
| pre-freeze | 15 hr | 95.6% | 0.2% | 0.01% | 3.3% | 0.9% |
| | 18 hr | 96.4% | 0.1% | 0.0% | 2.7% | 0.9% |
| | 21 hr | 96.3% | 0.2% | 0.0% | 2.3% | 1.2% |
| | 24 hr | 96.2% | 0.2% | 0.0% | 2.2% | 1.5% |
| | 24 hr UTD | 96.4% | 0.1% | 0.06% | 2.4% | 1.1% |
| | (n = 2) | | | | | |
| | 12 hr (of | 97.1% | 0.6% | 0.0% | 2.4% | 0.0% |
| | CAR+ only) | | | | | |
| | 15 hr (of | 96.6% | 0.9% | 0.0% | 2.5% | 0.0% |
| | CAR+ only) | | | | | |
| | 18 hr (of | 99.2% | 0.1% | 0.0% | 0.7% | 0.0% |
| | CAR+ only) | | | | | |
| | 21 hr (of | 99.1% | 0.3% | 0.0% | 0.7% | 0.0% |
| | CAR+ only) | | | | | |
| | 24 hr (of | 98.9% | 0.3% | 0.0% | 0.8% | 0.0% |
| | CAR+ only) | | | | | |

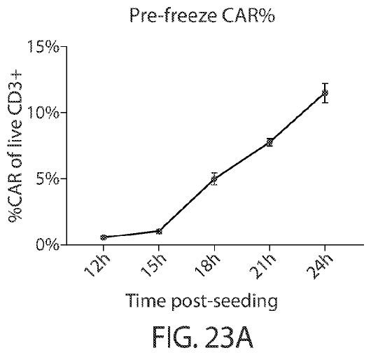

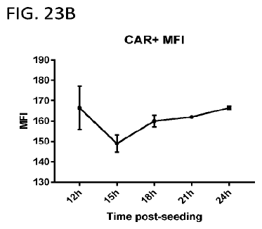

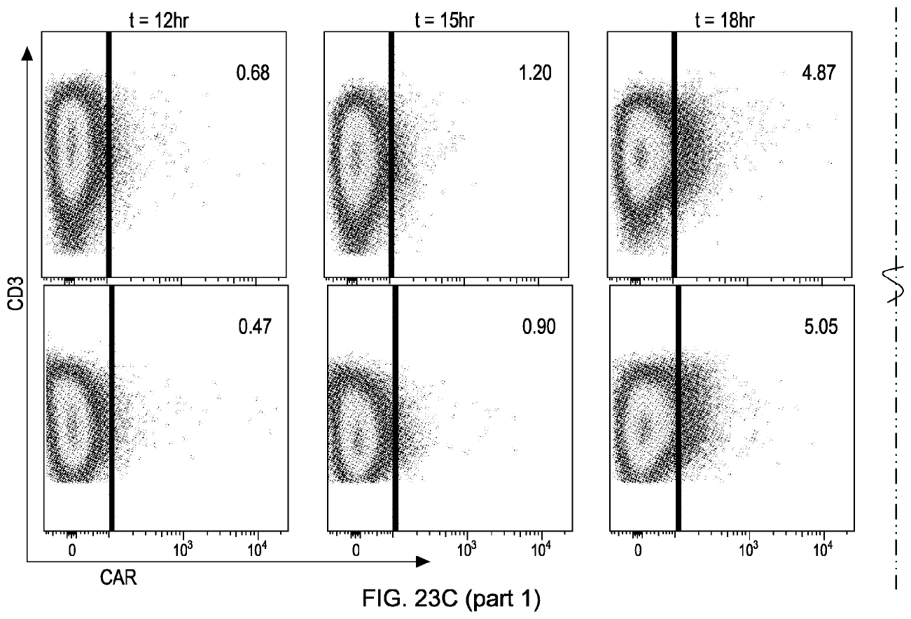

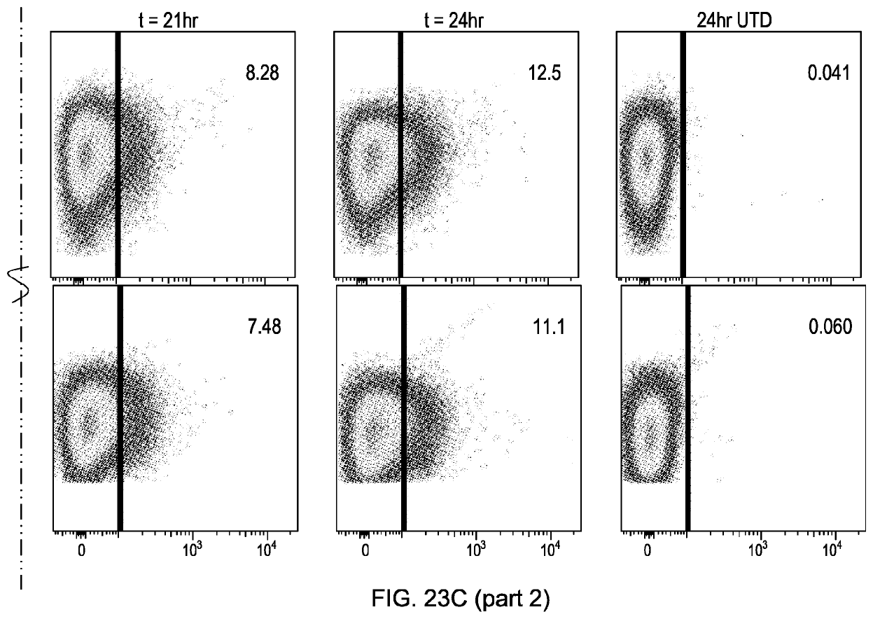

The increase in purity of CAR-expressing cells 18 hours into culture (Table 22) coincides with an increase in the percentage of T cells with CAR surface expression (FIGS. 23A and 23C). As observed previously with rapid CART products evaluated by flow cytometry after 24 hours in culture (see Example 5), CAR surface expression did not lead to distinct positive and negative populations. Gating for CAR positivity was therefore established using the UTD samples as the lower bound. The proportion of CD3+ cells expressing extracellular CAR remained below 1% until 15 hours post-seeding; and CAR expression then increased 3-4% every three hours to a maximum of 11.8% without saturating (FIG. 23A). The intensity of CAR expression as determined by MFI also increased slightly >18 hours in culture but remained dim through hour 24 (FIG. 23B).

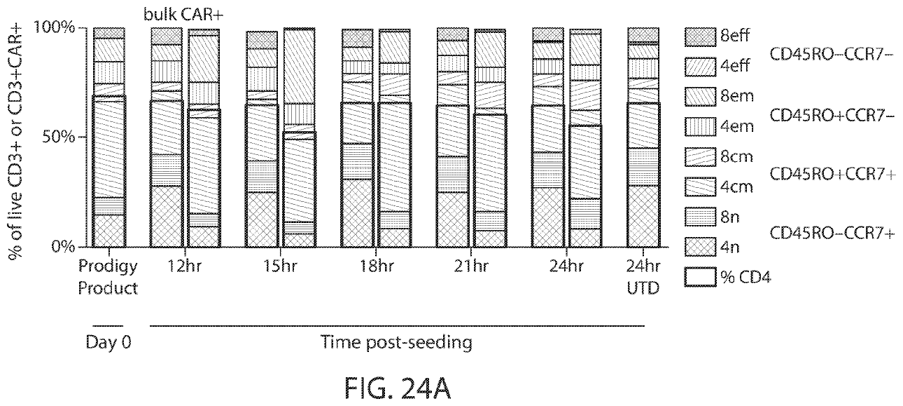

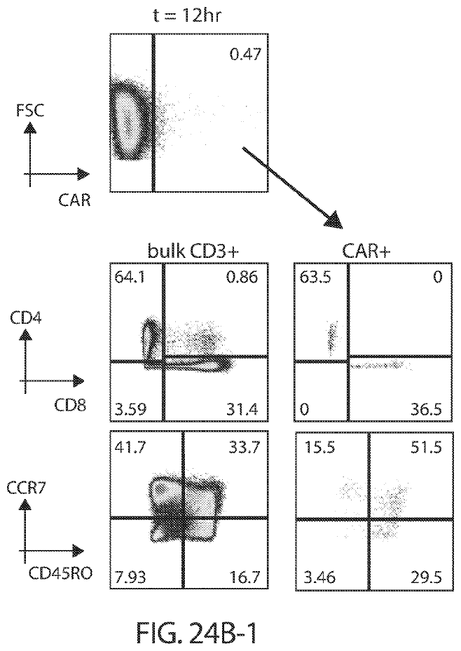

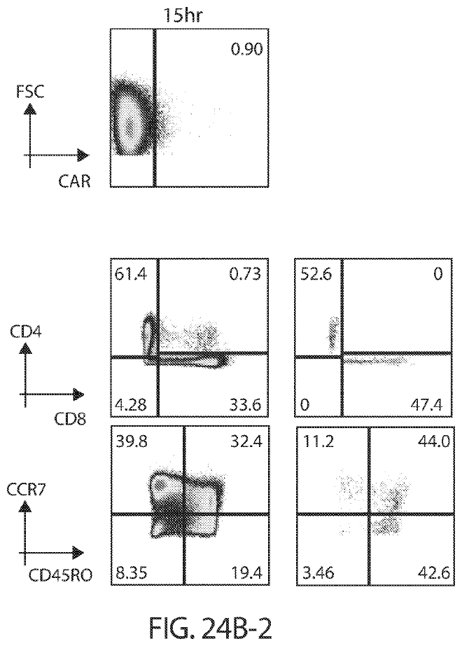

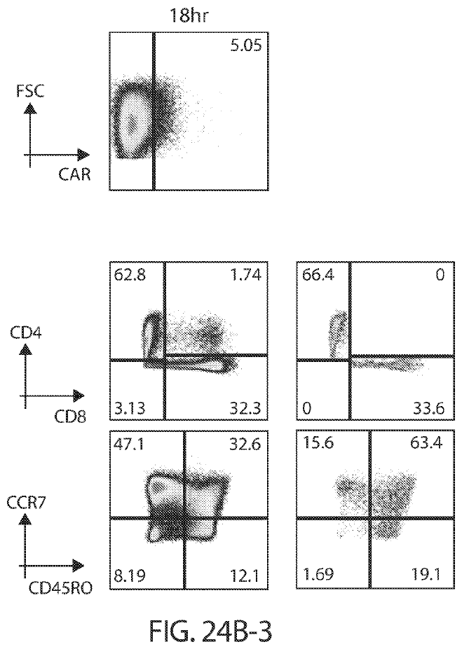

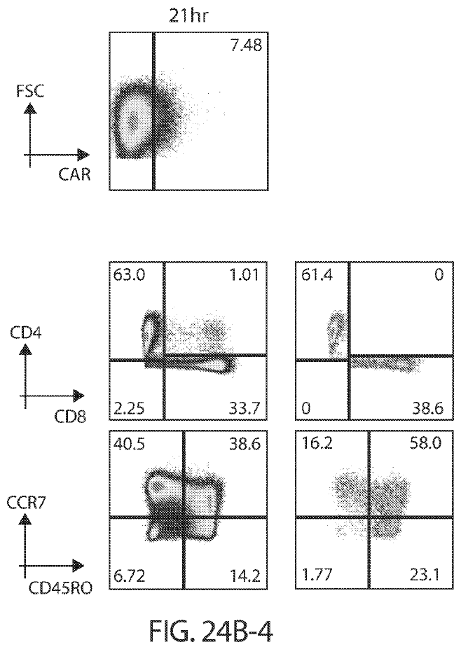

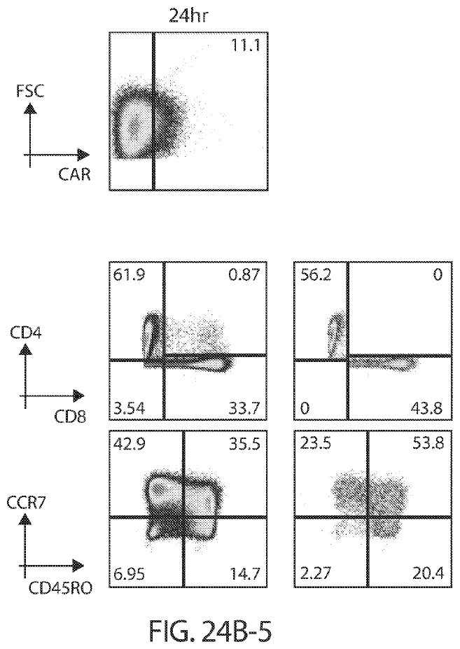

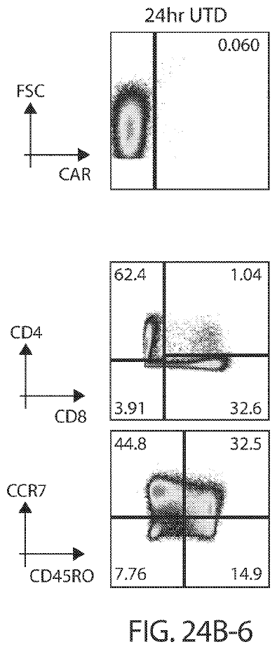

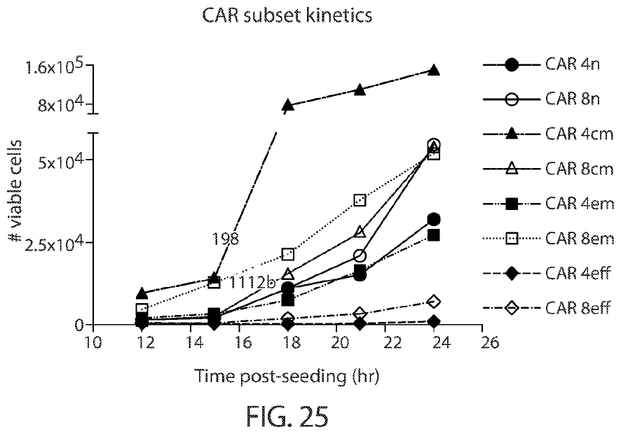

T cell subsets (CD4:CD8 ratio and memory subset composition) were also evaluated at each timepoint (FIGS. 24A and 24B) using a combination of CD4, CD8, CD45RO, and CCR7; where undifferentiated naïve-like T cells were defined as CCR7+CD45RO−, central memory cells as CCR7+CD45RO+, effector memory cells as CCR7−CD45RO+, and highly differentiated effector T cells as CCR7−CD45RO−. Across all timepoints evaluated, including the UTD, cultures contained a greater proportion of naïve cells (40-47%) and lower proportion of central memory cells (33-39%) than the initial starting material (23% and 52%, respectively). Interestingly, although the frequency of naïve or central memory T cells in the bulk composition did not change between 12 to 24 hours, later harvests were correlated with a greater frequency of extracellular CAR-expressing cells that were naïve and a lower frequency of extracellular CAR-expressing cells that were central memory (16% naïve/63% central memory among CAR-expressing cells at 18 hours vs. 24% naïve/54% central memory among CAR-expressing cells at 24 hours). Similarly, while bulk CD4:CD8 ratio did not change significantly, the CD4 fraction of the CAR+ cells decreased by 10% (66% to 56%) between 18-24 hours. Converting these frequencies to total cell numbers (FIG. 25) reveals that the subsets of T cells that appeared to express the CAR the earliest are mostly naïve CD4 cells between 15-18 hours in culture; naïve CD8 CARs and central memory CD8 CARs then rapidly increase in frequency.

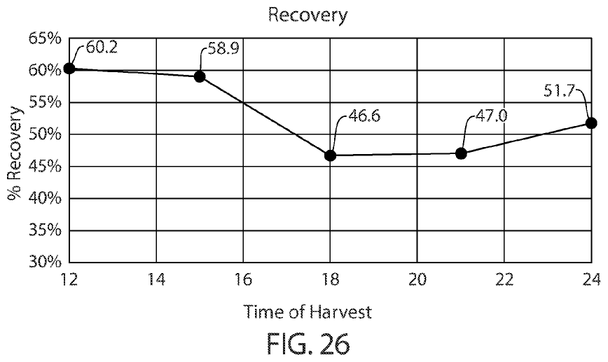

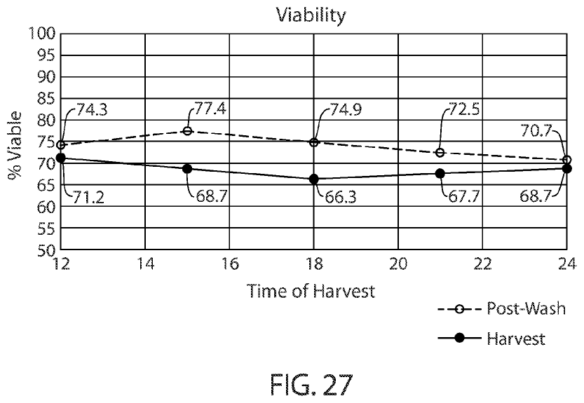

Viable cell recovery (or fold expansion) as well as pre- and post-wash viability were determined at each harvest time point (FIGS. 26 and 27). Recovery of viable cells decreased by 13% until 18 hours post-seeding (lowest 46%, coinciding with the increased rate of extracellular CAR expression), then increased slightly to 52% for cultures harvested at later time points (FIG. 26). Product viability increased after washing to 71-77% with viability decreasing for harvests between 15-24 hours (FIG. 27).

### Conclusion

Of time points tested between 12-24 hours, rapid CARTs seeded simultaneously with TransAct and technical grade CTL019 vector show the highest CAR surface expression at 24 hours. Very few cells are CAR+ (as measured at the time of harvest) until 15 hours post-seeding, after which % CAR increases more rapidly. The intensity of CAR expression is dim but increases slowly after 18 hours post-seeding.

Rapid CART products become purer (greater % T cells) than the starting material at all points between 12 to 24 hours post-seeding due to monocyte loss in the first 12 hours, followed by a minor loss of NK cells and any residual B cells not removed by Prodigy® enrichment.

Although overall cell recovery is lowest when harvested 18 hours post-seeding (improving slightly by 24 hours), the overall T cell composition does not change between 12 and 24 hours post-seeding. T cells that first express extracellular CAR are mostly central memory CD4s between 15 and 18 hours post-seeding, then naïve and central memory CD8s show CAR expression.

### Example 7

### Description of the Activated Rapid Manufacturing (ARM) Process

In some embodiments, CART cells are manufactured using a continuous Activated Rapid Manufacturing (ARM) process, over approximately 2 days, which will potentially allow for a greater number of less differentiated T cells (T naïve and T_{SCM} (stem central memory T) cells) to be returned to a patient for in vivo cellular expansion. The short manufacturing time period allows the early differentiated T cells profile to proliferate in the body for their desired terminal differentiated state rather that in an ex vivo culture vessel.

In some embodiments, CART cells are manufactured using cryopreserved leukapheresis source material, for example, non-mobilized autologous peripheral blood leukapheresis (LKPK) material. Cryopreserved source material undergoes processing steps for T cell enrichment on the first day of production (Day 0) by means of anti-CD4/anti-CD8 immunomagnetic system. Positive fraction is then seeded in G-rex culture vessel, activated with an anti-CD3/CD28 system (TransACT) and on the same day transduced with a lentiviral vector (LV) encoding a CAR. On the following day, after 20-28 hours of transduction, the T cells are harvested, washed four times, formulated in freezing medium and then frozen by a Controlled Rate Freezer (CRF). From the start of the process on Day 0 to the initiation of harvest on the following day, cells are cultured for 20-28 hours with a target of 24 hours after Day 0 seeding.

Media for Day 0 were prepared according to Table 21. The cryopreserved leukapheresis material is thawed. The thawed cells are diluted with the Rapid Buffer (Table 21) and washed on the CliniMACS® Prodigy® device. The T cells are selected by CliniMACS® CD4 and CD8 microbeads. Once the program is finished for T cell selection (approximately 3 h 40 min to 4 h 40 min), the reapplication bag containing the cells suspended in Rapid Media (Table 21) are transferred in a transfer pack. A sample is taken for viability and cell count. The cell count and viability data from the positive fraction bag is used to determine the cell concentration when seeding the culture vessel for activation and vector transduction.

Following positive selection of T cells via the CliniMACS® microbeads (CD4 and CD8), the cells are seeded in the culture vessel, G-Rex. Once the cells are seeded, the activation reagent (TransACT) is then added to the culture vessel. The cells are then transduced with a lentiviral vector encoding a CAR at a target MOI of 1.0 (0.8-1.2). Following the vector addition, the culture vessel is transported to an incubator where it is incubated for a target of 24 hours (operating range 20-28 hours) at a nominal temperature of 37° C. (operating range 36-38° C.) with nominal 5% CO₂ (operating range 4.5-5.5%). Following the incubation, the cells are washed with Harvest Wash Solution (Table 21) four times to remove any non-integrated vector and residual viral particles, as well as any other process related impurities. Then, the cells are eluted and a sample for cell count and viability is taken for testing and the results are used to determine the volume required to re-suspend the cells for final formulation with CryoStor® CS10. The cells are then centrifugated to remove the Harvest Wash Solution and proceed with cryopreservation.

In some embodiments, the CAR expressed in CART cells binds to CD19. In some embodiments, IL-2 used in the Rapid Media (RM) (Table 21) can be replaced with IL-15, hetIL-15 (IL-15/sIL-15Ra), IL-6, or IL-6/sIL-6Ra.

In some embodiments, the CAR expressed in CART cells binds to BCMA. In some embodiments, IL-2 used in the Rapid Media (RM) (Table 21) can be replaced with IL-15, hetIL-15 (IL-15/sIL-15Ra), IL-6, or IL-6/sIL-6Ra.

### Example 8

### Characterization of CD19 CART Cells Manufactured Using the Activated Rapid Manufacturing (ARM) Process

Disclosed herein is an anti-CD19 CAR-T cell product manufactured using the activated rapid manufacturing (ARM) process. The ARM process reduces the turnaround time compared to traditional manufacturing (TM) processes, prospectively allowing a timely infusion of the anti-CD19 CAR-T cell product to patients. Moreover, the ARM process also preserves putative stem memory T (Tₛₜₑₘ) cells, a cellular subset associated with improved antitumor efficacy. The main difference in manufacturing is that while the TM process includes an expansion phase in which anti-CD19 CART cells are cultured in vitro for 9 days with interleukin (IL-) 2 before being formulated, the ARM process allows formulation after only 24 hours of culture. This is made possible by the use of a fully biocompatible nanomatrix coupled to monoclonal antibodies (mAb) with agonistic activity against CD3 and CD28, which differently from the CD3/CD28 paramagnetic beads used in the TM process, can be washed away with the residual lentiviral vector right after transduction. Results from a xenograft mouse model, as well as final product enrichment for Tₛₜₑₘ cells, a subpopulation associated with increased persistence and long-term antitumor effects, suggest an overall improved therapeutic potential of anti-CD19 CAR T cells manufactured using the ARM process as compared to anti-CD19 CART cells manufactured using the TM process. Another important difference revealed by the xenograft mouse model is a potential delayed cellular kinetics expansion of anti-CD19 CART cells manufactured using the ARM process for approximately one week compared to the counterparts manufactured using the TM process. This delay is estimated to be approximately 1 week, which imposes corresponding prolongation of the window for careful monitoring of potential toxicities from 3 weeks, as with anti-CD19 CART cells manufactured using the TM process, to 4 weeks. Conversely, non-clinical safety data from an in vitro cytokine release model indicate that anti-CD19 CART cells manufactured using the ARM process and those manufactured using the TM process might have a similar potential to induce IL-6 production in vivo and therefore carry a similar cytokine release syndrome (CRS) risk. Based on this evidence, anti-CD19 CAR T cells manufactured using the ARM process will be investigated in a Phase I, open label clinical study in patients with advanced small lymphocytic lymphoma (SLL)/chronic lymphocytic leukemia (CLL) in combination with the Bruton tyrosine kinase inhibitor (BTKi) ibrutinib (Imbruvica), an already approved drug in this indication, and as single agent in DLBCL.

### Generation and In Vitro Analysis

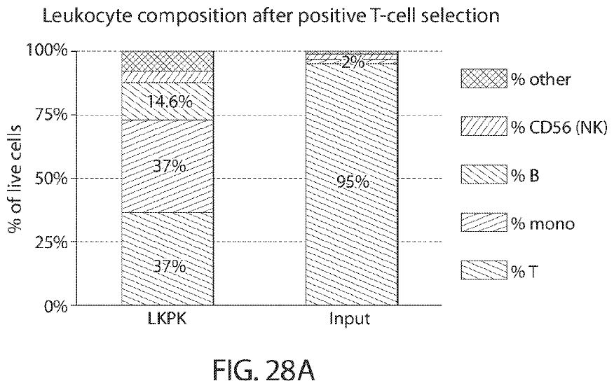

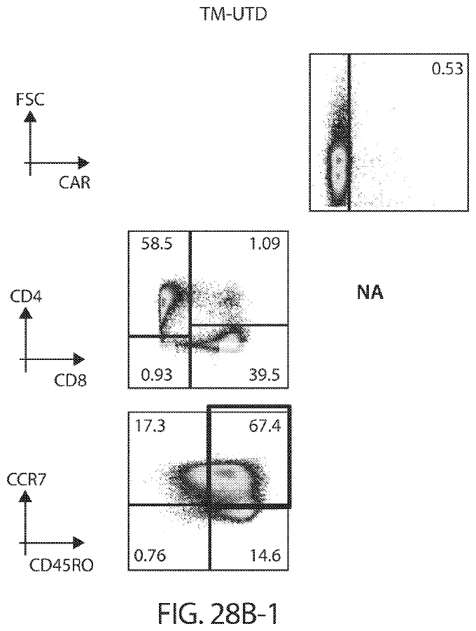

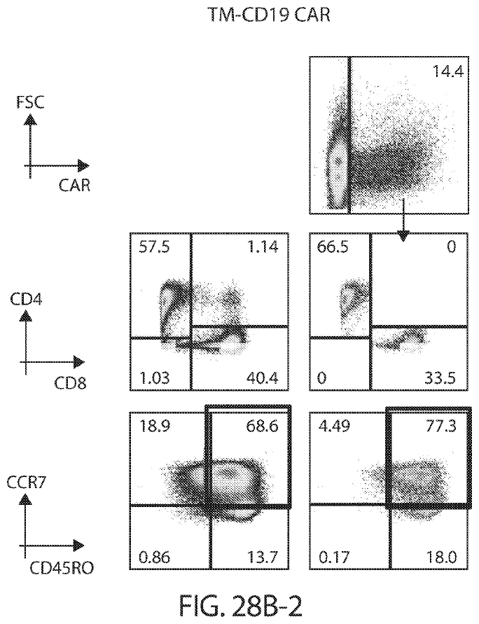

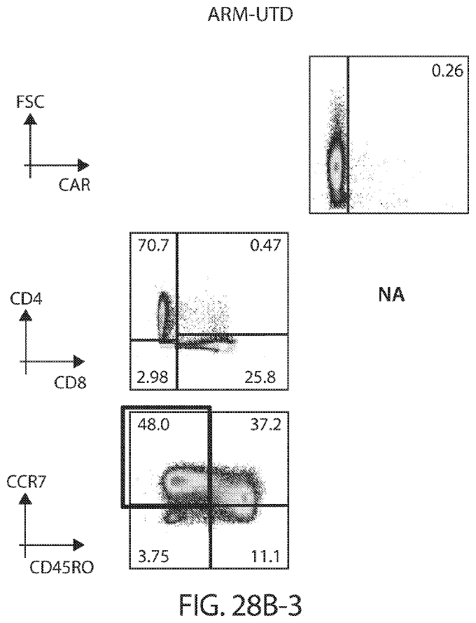

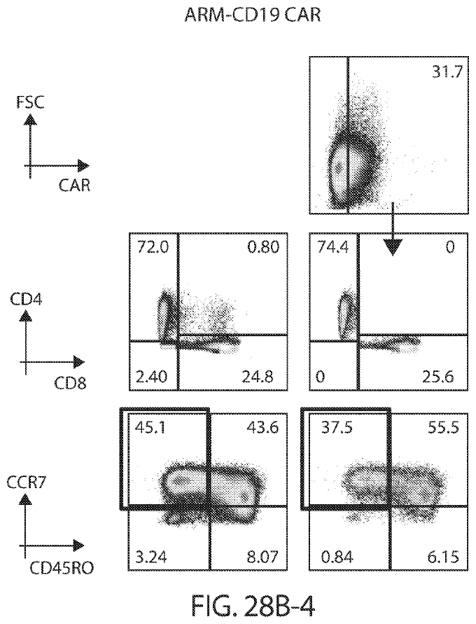

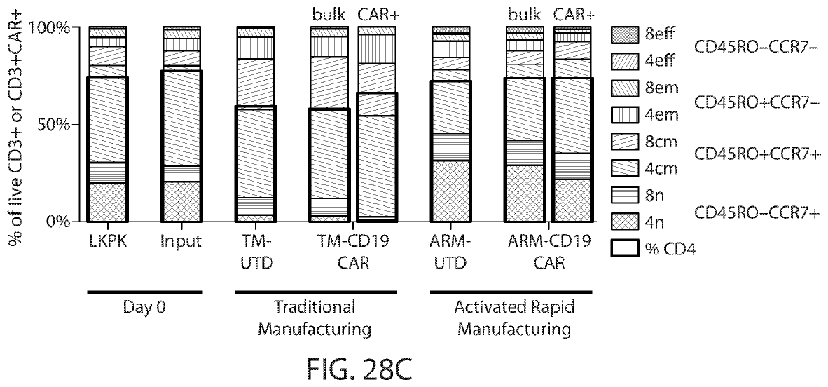

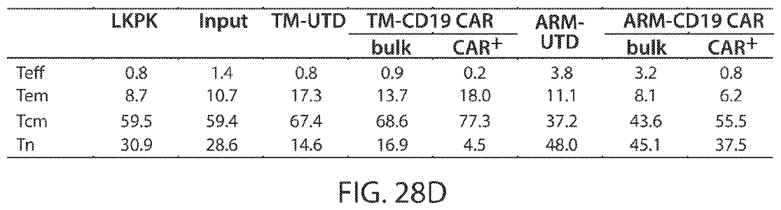

To test the ARM process for anti-CD19 CART cell manufacturing at clinical scale, a frozen healthy donor leukapheresis product (Leukopak, LKPK) was used as starting material, described in FIG. 28A as a representative example. The LKPK contained 37% T cells, 4% NK cells, 37% monocytes and 15% B cells (FIG. 28A). After thawing, T cells were positively selected using anti-CD4 and anti-CD8 microbeads. The composition of the product after positive T cell selection was 95.4% T cells, 1.9% NK cells, 1.7% monocytes, and 0.1% B cells (FIG. 28A).

Positively selected T cells were activated using a polymeric nanomatrix conjugated to anti-CD3 and anti-CD28 agonist monoclonal antibodies and transduced with a lentiviral vector encoding anti-CD19 CAR. After 24 hours in culture, cells were harvested and cryo-preserved (such cells are referred to as “ARM-CD19 CAR” in this example). In parallel, CAR-T cells were generated using a traditional manufacturing (TM) process (such cells are referred to as “TM-CD19 CAR” in this example), using the same donor T cells and lentiviral vector. The TM process utilized paramagnetic beads coupled to anti-CD3 and anti-CD28 antibodies and a 9-day culture period in tissue-culture flasks, followed by the same harvest and freezing procedure. CAR-T cells generated by each process were analyzed by flow cytometry to evaluate CAR expression post thaw, as well as the Tcell phenotype (FIGS. 28B-28D). Analysis of the T-cell phenotype revealed that the ARM process retained naïve-like T cells (45.1% CD45RO−/CCR7+) in both the CD8 and CD4 compartments, while the TM process mainly resulted in central-memory T (T_{CM}) cells (68.6% CD45RO+/CCR7+ compared to 43.6% for ARM-CD19 CAR) (FIGS. 28C and 28D). Importantly, the ARM process better maintained the initial naïve-like CD45RO−/CCR7+ T-cell population as compared to the TM process, also in the CAR+ population (28.6% in starting material, 37.5% for ARM-CD19 CAR and 4.5% for TM-CD19 CAR) (FIGS. 28C and 28D). This T-cell population largely overlaps with CD45RO−/CD27+ Tstem cells described by Fraietta, et al (2018) Nat Med, 24(5); 563-571 and associated with sustained remission in a CLL phase I clinical trial.

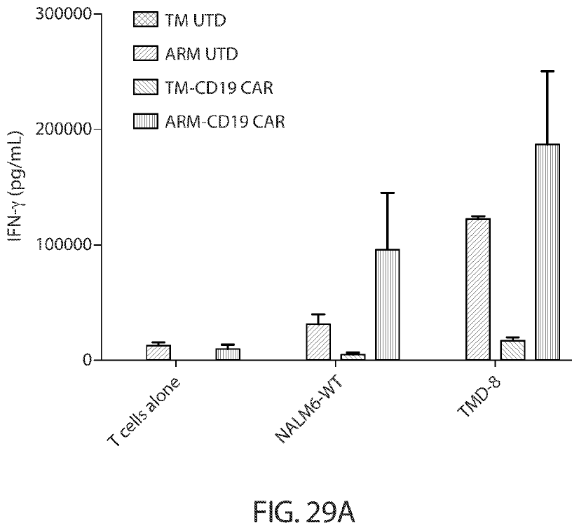

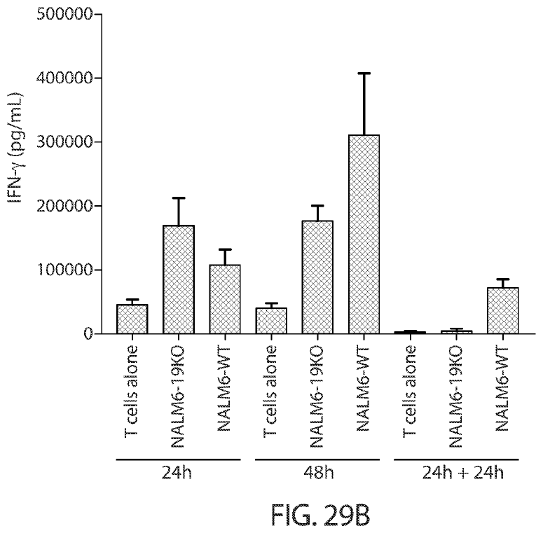

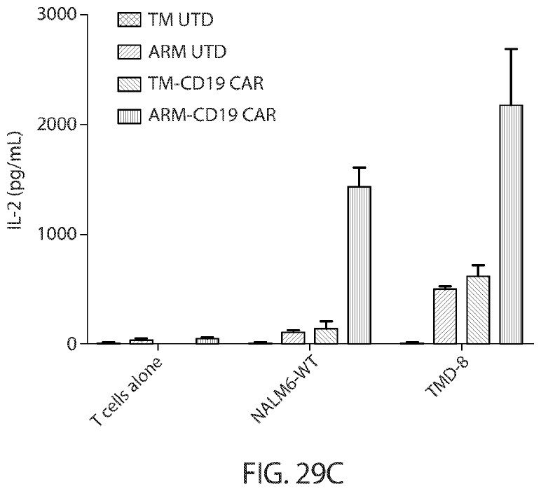

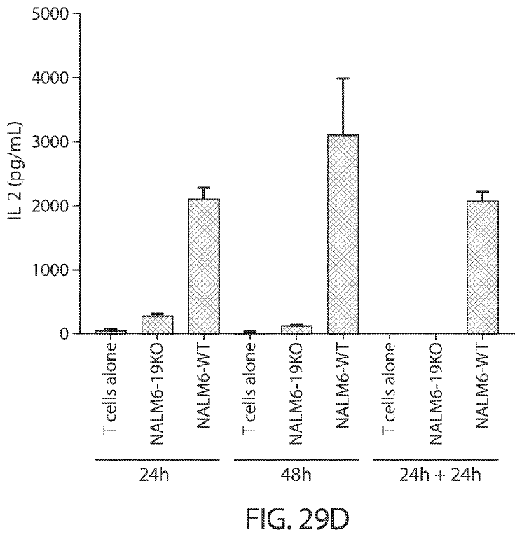

In addition to its phenotype, the final ARM-CD19 CAR cell product was also assessed for its function in vitro. ARM-CD19 CAR and TM-CD19 CAR were thawed and co-cultured with the CD19-expressing cell lines NALM6 (ALL) or TMD-8 (DLBCL). Comparison of cytokine levels in the supernatants 48 hours after co-culture revealed a 11- to 17-fold increase of IFN-γ and a 3.5- to 10-fold increase in levels of IL-2 secreted by ARM-CD19 CAR as compared to TM-CD19 CAR, depending on the stimulating cancer cells (NALM6 or TMD-8, FIGS. 29A and 29C). Experiments with untransduced (UTD) cells that underwent the ARM or TM process (FIG. 29C), or with CD19-negative NALM6 (NALM6-19K0) target cells (FIG. 29D) confirmed CD19-specific recognition by ARM-CD19 CAR and TM-CD19 CAR. Higher background of IFN-γ secretion by ARM-UTD and ARM-CD19 CAR in the absence of CD19-specific stimulation (FIGS. 29A and 29B, respectively) is likely due to the activated nature of these products. This background secretion decreased by 48 hours of coculture (FIGS. 29B and 29D). An intermediate wash of the cells after the first 24 hours of coculture with target cells, followed by co-culture for additional 24 hours (24 h+24 h) further enhanced the difference between background and CD19-specific cytokine secretion. This 24 h+24 h condition highlights that background IFN-γ secretion by ARM-CD19 CAR abates after the first 24 hours.

In summary, the ARM process used to generate ARM-CD19 CAR results in T cells with CAR-expression similar or higher than that of TM-CD19 CAR. Importantly, the ARM process maintains a T-cell phenotype similar to the input material. ARM-CD19 CAR demonstrates CD19-specific activation in vitro, and secretes higher levels of IL-2 as compared to TM-CD19 CAR, which correlates with its Tₛₜₑₘ phenotype.

### In Vivo Efficacy

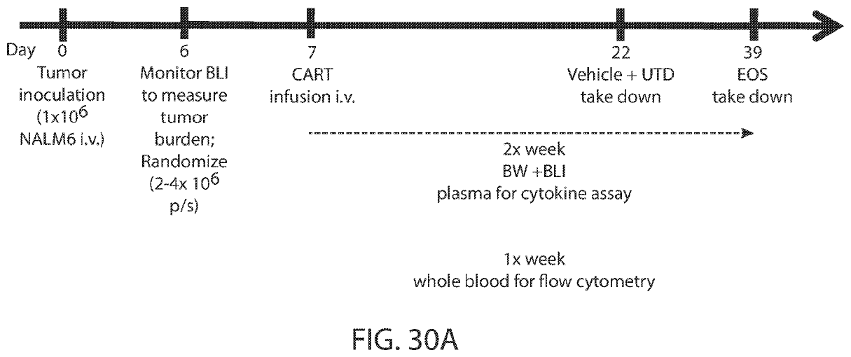

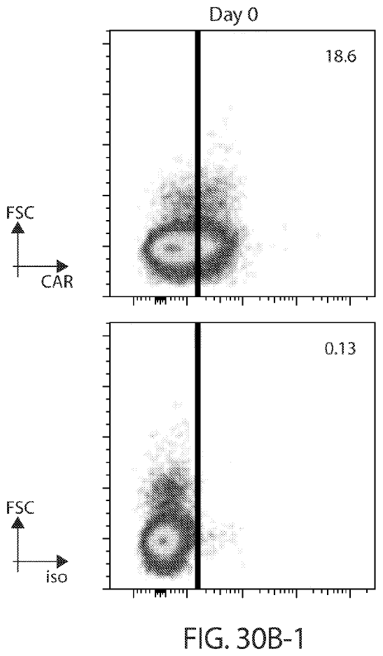

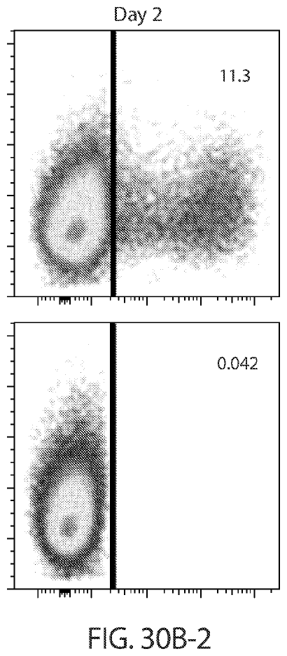

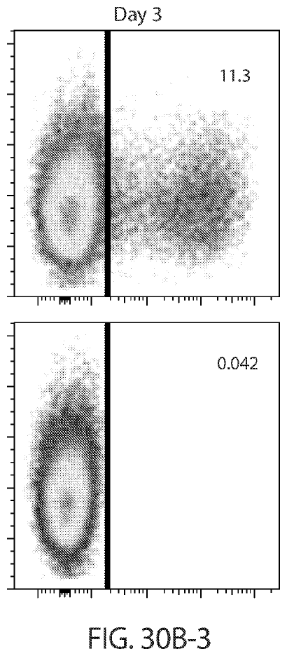

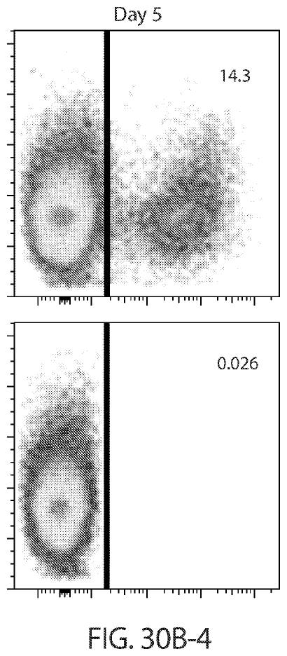

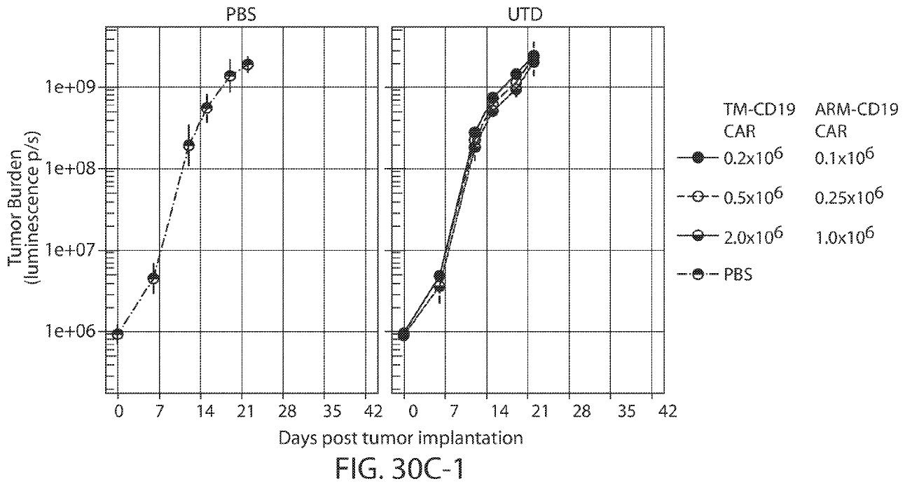

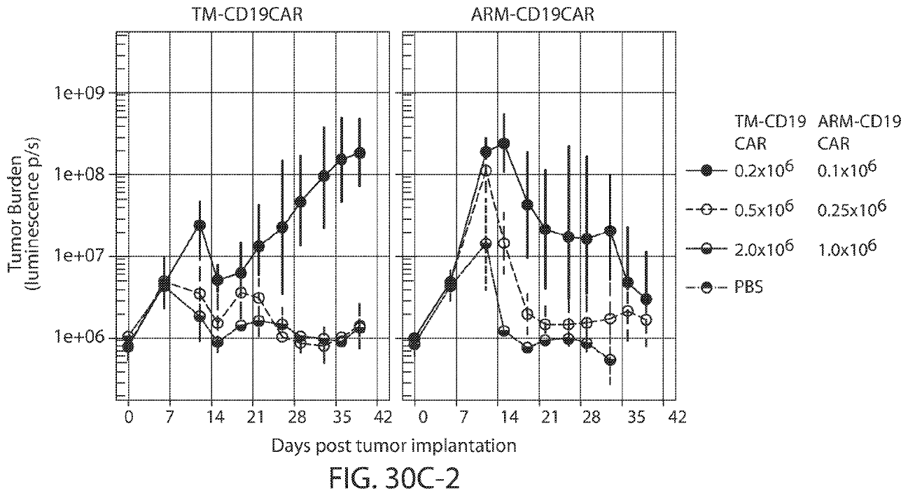

Efficacy studies in vivo were used to guide the development of the ARM process, ultimately leading to the process that will be used for clinical anti-CD19 CART cell manufacturing. For the experiment described here, ARM-CD19 CAR was generated at clinical scale. In parallel, TM-CD19 CAR was generated using the same lentiviral vector and T cells from the same donor. The efficacy of CAR-T cells generated using the different processes was evaluated in immunodeficient NSG mice (NOD-scid IL2Rg-null), which were inoculated with the pre-B ALL cell line NALM6. This tumor cell line engrafts in the bone marrow, but in case of high tumor burdens can also be detected in the circulation. Seven days after leukemia inoculation, cohorts of mice received a single infusion of CAR+ T cells (FIG. 30A). Planned doses of 0.2×10⁶, 0.5 ×10⁶ and 2×10⁶ viable CAR+ T cells were determined based on post thaw flow analysis of TM-CD19 CAR and ARM-CD19 CAR on day 0.

Because of the concern of pseudo-transduction for ARM-CD19 CAR on day 0 post thaw, a sentinel vial was thawed and cultured for up to 5 days, and CAR expression (percentage and mean fluorescence intensity) was analyzed by flow cytometry at different time points (FIG. 30B). The percentage of positive cells on later time points was lower as compared to the day 0 post-thaw sample. At the same time, CAR mean fluorescence intensity was higher per cell, reflective of stably transduced CAR-T cells. The measurement on day 3 was used to determine the actual dose of ARM-CD19 CAR, which was determined to be 0.1×10⁶, 0.25 ×10⁶ and 1×10⁶ viable CAR+ T cells. The TM-CD19 CAR dose remained unchanged (0.2×10⁶, 0.5 ×10⁶ and 2×10⁶ viable CAR+ T cells), as the flow analysis of post-thaw samples was performed on rested, fully integrated CART cells.

Both ARM-CD19 CAR and TM-CD19 CAR induced tumor-regression in a dose-dependent manner (FIG. 30C). Mice treated with 0.5×10⁶ or 2×10⁶ TM-CD19 CAR cells, or 0.25 ×10⁶ or 1×10⁶ ARM-CD19 CAR cells, experienced durable tumor regression. Interestingly, at the respective lowest dose tested (0.2 ×10⁶ TM-CD19 CAR cells or 0.1×10⁶ ARM-CD19 CAR cells), response to TM-CD19 CAR was not sustained and all mice eventually relapsed after initial partial leukemia control. In contrast, at the lowest dose (0.1×10⁶) ARM-CD19 CAR-treated mice showed a steady decline of tumor burden that lasted until the end of study. The kinetics of tumor regression suggest a delayed activation of ARM-CD19 CAR by about 1 week, suggesting that Tₛₜₑₘ cells need to proliferate and differentiate into effector cells in order to exert their antitumor activity.

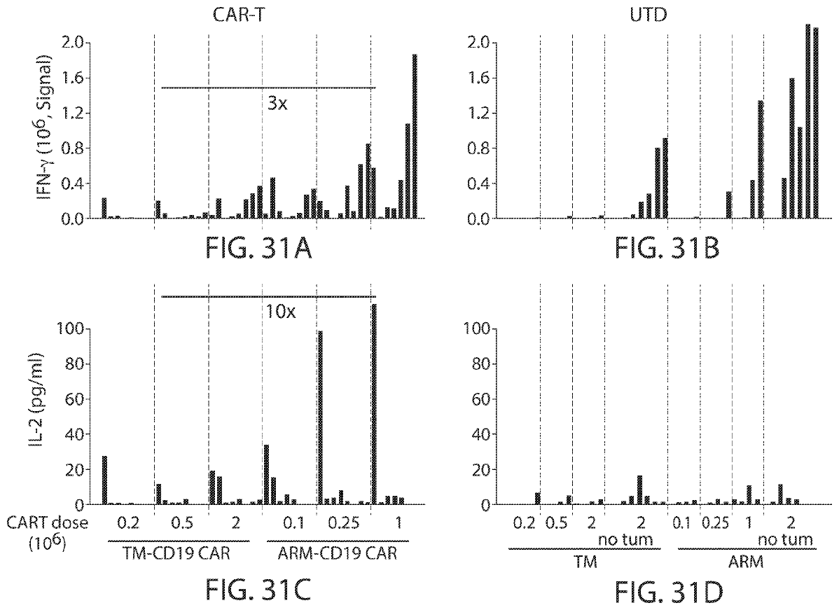

Mice treated with CAR-T cells and UTD cells generated by the two manufacturing processes were bled twice weekly to measure cytokine levels (FIGS. 31A-31D). Circulating IFN-γ levels in mice infused with CAR-T cells, either ARM-CD19 CAR or TM-CD19 CAR, showed a bi-phasic pattern (FIG. 31A). An early IFN-γ peak was observed at days 4-7 after CAR-T cell infusion and likely related to CD19-specific activation following tumor recognition, since it was not evident in mice infused with TM-UTD or ARM-UTD (FIG. 31B). Early CD19-mediated activation was confirmed by a concomitant rise of in vivo IL-2 levels (FIG. 31C), which however abated at later time points.

### In Vivo Cellular Kinetics

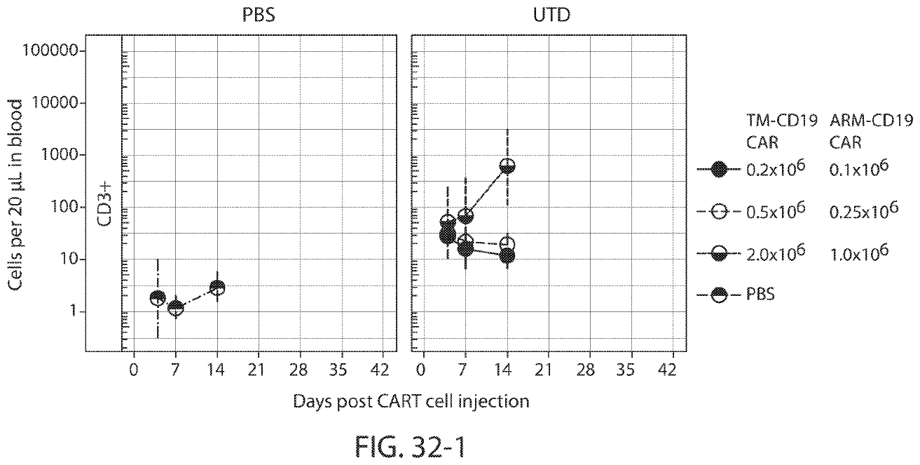

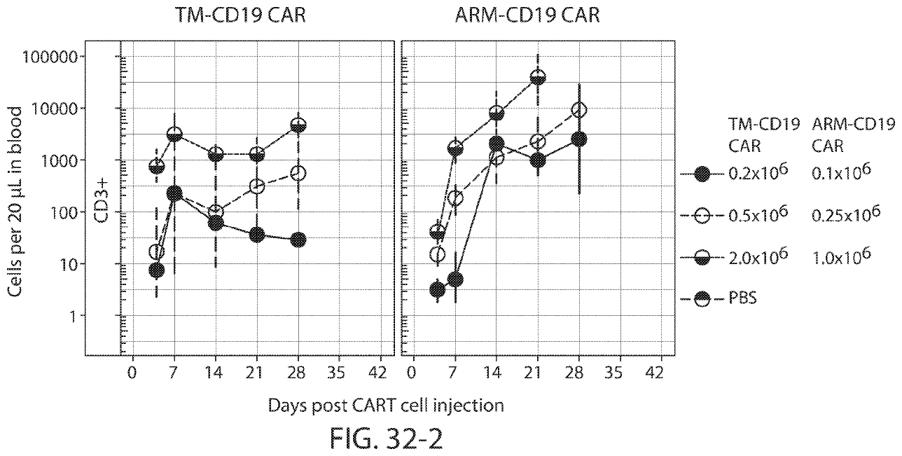

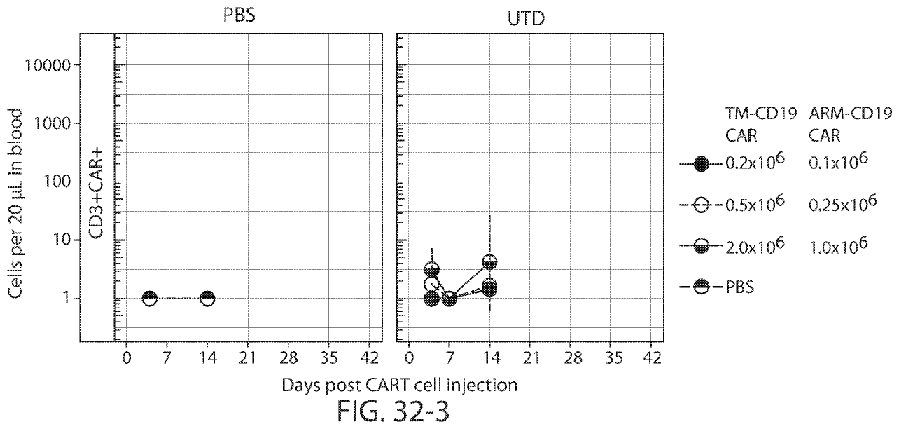

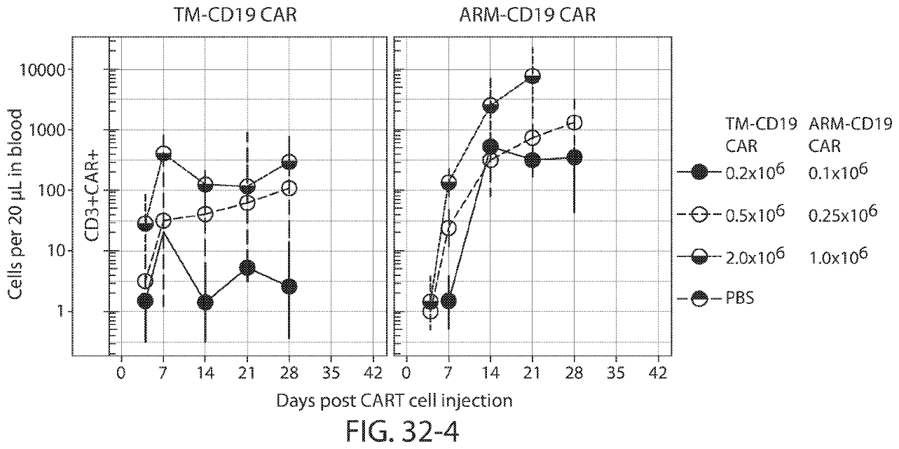

As part of a pharmacology study to evaluate the efficacy of ARM-CD19 CAR in NSG mice, the expansion of CAR+ T cells was assessed in vivo (FIG. 32). CD3+/CAR+ T-cell concentration in blood was analyzed by flow cytometry up to 4 weeks after infusion. CAR-T cell expansion can be inferred. However, long-term persistence cannot be assessed due to limited study time dictated by onset of X-GVHD. Cellular expansion was observed for both ARM-CD19 CAR and TM-CD19 CAR at all doses, except for TM-CD19 CAR at the lowest dose of 0.2 ×10⁶ cells. Exposure (Cmax and AUC within 21 days post cell injection) increased with increasing dose for both TM-CD19 CAR and ARM-CD19 CAR. To compare the expansion of ARM-CD19 CAR to TM-CD19 CAR at the same dose level, exposure of TM-CD19 CAR was interpolated to comparable doses of ARM-CD19 CAR (0.25×10⁶ and 1×10⁶ cells). The Cmax was 24- to 46-times higher and the AUCO-21d was 18- to 33-times higher compared to TM-CD19 CAR at doses of 0.25 ×10⁶ and 1×10⁶ cells. The time to ARM-CD19 CAR peak expansion (Tmax) was delayed for at least 1 week compared to TM-CD19 CAR.

In summary, pharmacology studies evaluating ARM-CD19 CAR in vitro show that ARM-CD19 CAR has an early-differentiated phenotype and has the potential to secrete more IFN-γ and IL-2. In vivo, ARM-CD19 CAR demonstrated delayed but higher cellular expansion, induced more IL-2 secretion, and controlled tumor growth at lower doses as compared to TM-CD19 CAR. Other features of ARM-CD19 CAR discussed, such as elevated levels of plasma IFN-γ at later time points and earlier occurrence of X-GVHD were seen both for ARM-CD19 CAR, as well as for ARM-UTD, underlying the limitations of the xenograft mouse model used here. Together, these results support the hypothesis that ARM-CD19 CAR contains T cells with more stemness features, enabling ARM-CD19 CAR to effectively engraft, expand and reject tumors.

### In Vitro IL-6 Release Assay

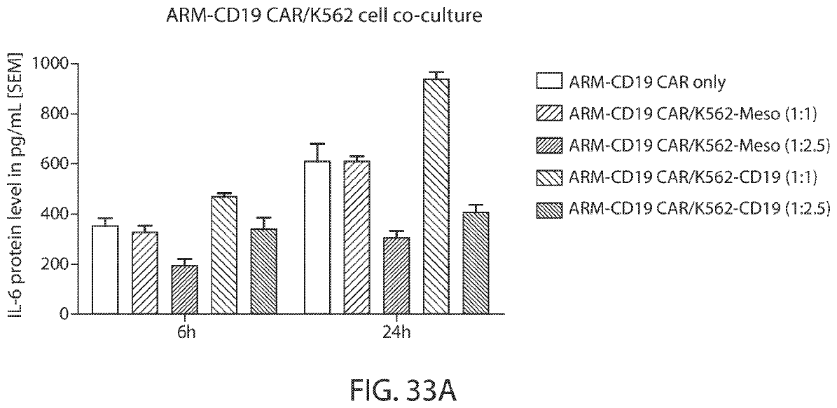

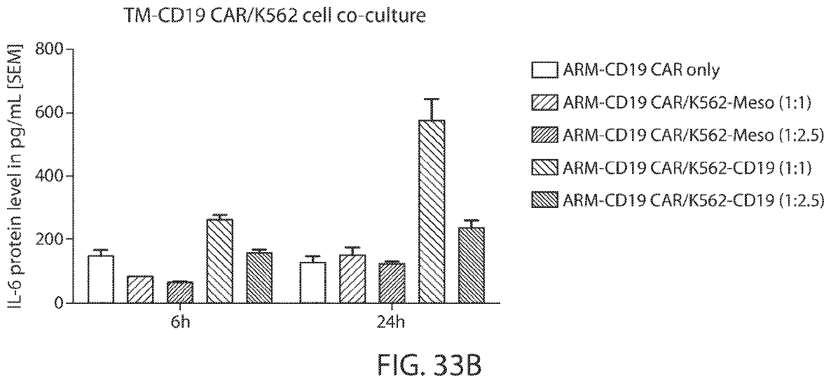

A three-party co-culture model for the in vitro investigation of IL-6 induction potential by CART cells was first published by Norelli, et al (2018) Nat Med., June; 24(6); 739-748 and applied here with some adaptations. This model consists of CAR-T cells, leukemic target cells and bystander THP-1 monocytic cells, as a source of myeloid cells for maximized IL-6 production. In this in vitro cellular model, IL-6 secretion by either ARM-CD19 CAR or TM-CD19 CAR alone was increased by co-culturing with CD19-expressing targets and THP-1 cells (FIGS. 33A and 33B). Importantly, time-dependent CD19-specific IL-6 secretion induced by ARM-CD19 CAR was superimposable to that induced by TM-CD19 CAR. In the same in vitro model, CD19-specific IFN-γ secretion in the ARM-CD19 CAR condition was 10-fold higher than in the TM-CD19 CAR condition (data not shown).

### Summary

These results suggest that ARM-CD19 CAR might have greater antitumor potential and a similar safety profile as compared to TM-CD19 CAR. Greater antitumor potential is inferred by better tumor control at the lowest dose tested and by higher in vivo cellular expansion. Such a calculation may however be an underestimation of the overall therapeutic potential of ARM-CD19 CAR, since this was assayed in an ALL model (NALM6) which is more aggressive than the two disease indications (CLL and DLBCL) in which ARM-CD19 CAR will be initially investigated. In CLL, in particular, where in vivo CAR-T cell expansion robustly correlates with tumor regression (Mueller, et al (2017) Blood. 130(21); 2317-2325; Fraietta, et al (2018) Nat Med, 24(5); 563-571), significantly higher proliferative potential of ARM-CD19 CAR (up to 20-fold) might result in meaningful superior efficacy compared to TM-CD19 CAR.

In mice, the early systemic release of IFN-γ and IL-2 by ARM-CD19 CAR associated with CAR-mediated tumor regression was 3-fold and 10-fold higher than that induced by traditionally manufactured CAR-T cells, respectively. IL-6 levels were not studied in vivo, since in this strain lack of functional myeloid cells results in the inability to produce inflammatory cytokines (Norelli, et al (2018) Nat Med., June; 24(6); 739-748; Giavridis, et al (2018) Nat Med., June; 24(6);731-738). To obviate this and evaluate the potential for in vivo IL-6 release induced by ARM-CD19 CAR, an in vitro three-party co-culture system was employed, in which bystander monocytic cells are added as a source of inflammatory cytokines (Norelli, et al (2018) Nat Med., June; 24(6); 739-748). In this system, IL-6 production was similar between ARM-CD19 CAR and traditionally manufactured CAR-T cells, suggesting a similar risk for CRS. Conversely, the delayed kinetics of ARM-CD19 CAR cellular expansion will require an extension of the CRS monitoring period from the 3 weeks typical of TM-CD19 CAR, to 4 weeks. In vitro experiments with ARM-CD19 CAR also revealed the potential for transient, non-CAR-mediated IFN-γ and IL-2 secretion by ARM-CD19 CAR during the first 3 days of culture after thawing. A comprehensive risk assessment based on data from patients receiving recombinant human IL-2 (Proleukin) and recombinant human IFN-γ (ACTIMMUNE), and taking in consideration the projected exposures following ARM-CD19 CAR infusion indicates that the risk for constitutional symptoms (fever, chills, erythema) as described in these patients, would be very low. To further mitigate this risk, patients receiving ARM-CD19 CAR will be hospitalized for at least 72 hours after infusion of the cellular product.

Finally, in the non-GLP compliant toxicology study, NSG mice engrafted with ARM-CD19 CAR did not show unexpected behavior in comparison to traditionally manufactured CAR-T cells and untransduced cells undergoing the ARM process, when assessed by blood or lymphatic organ immunophenotyping, as well as histological evaluation of a relevant set of organs.

### Example 9

### BCMA CART Cells Manufactured Using the ARM Process

### Methods

### T Cell Isolation

Fresh leukopak of healthy donor aphereses were obtained from Hemacare and stored in vapor phase liquid nitrogen (LN2) until needed. On Day 0, two quarter leukopaks were removed from LN2, warmed in the Plasmatherm (Barkey, Leopoldshohe, Germany) until a small ice crystal remained, and diluted with Prodigy® process buffer. Automated CD4/CD8 positive selection was then performed on the CliniMACS® Prodigy® with the TS 520 tubing set and T Cell Transduction (TCT) program software version 1.0. Cell count and viability for each Prodigy® output (product, waste, and nontarget cells) were determined by AO/PI staining as enumerated by the Cellometer Vision (Nexcelom, Lawrence, Mass.) to assess total cell recovery and T cell recovery. The CD4/CD8-enriched product was eluted in OpTmizer™ complete T cell medium and divided for further culturing using either the 24 h or traditional 9-day process (TM). Remaining T cells were frozen down in LN tank. T cell purity was evaluated by flow cytometry analyses.

### CAR-T Cells Production Using the ARM Process

T cells purified by Prodigy® were seeded into different scales of vessels, such as plate, flask, G-REX vessel or full clinical scale in centricult. Upon seeding, TransAct (Miltenyi Biotec)), a polymeric nanomatrix conjugated to anti-CD3 and anti-CD28 agonist, was added, in addition to clinical-grade lentiviral vector. Cells were incubated in OpTmizer™ complete T cell media containing 100 IU/mL human recombinant IL-2 (Prometheus, San Diego, Calif.), 2% ICRS (Life Technologies) for 24 h prior to harvest and cryopreservation.

Aliquots of cryopreserved CAR-T cells were thawed into pre-warmed OpTmizer™ complete media, washed twice with 20× volume of pre-warmed medium before culturing and flow cytometry analyses for assessing BCMA-CAR expression and stemness features at different time points post-thaw. Aliquots of the cell products were co-cultured with target cell lines to assess cytokine release in response to specific antigen stimulation.

### CAR-T Cells Production Using TM Process

Prodigy processed T cells were resuspended in warm RPMI complete T cell medium and plated in 24-well plates. T cells were incubated overnight at 37° C. with Human T-Expander CD3/CD28 beads at a 3:1 ratio of beads-to-cells.

On Day 1, lentiviruses were added at a MOI of 2, based on the SUP-Tltiter. No virus was added to the untransduced control (UTD). The T cells were incubated overnight at 37° C. followed by the addition of 1 mL complete T cell medium per well, after which they were incubated overnight at 37° C. For the remaining seven days of culture expansion, the T cells were transferred into tissue culture flasks and diluted with complete T cell medium every two days.

Between Days 8 to 9, the T cells were de-beaded, harvested and cryopreserved in CryoStor CS10 freezing medium, frozen at −80° C. in CoolCell Cell Freezing Containers (Biocision), and transferred to LN2 the following day. Small aliquots of T cells were stained for CAR expression. Single color controls were included for compensation. Samples were measured on a flow cytometer (BD LSRFortessa), and data were analyzed with FlowJo software.

### Target Cell Line and Culture

Nalm6 cells were transfected with a lentiviral firefly luciferase reporter construct to create the Nalm6-luc cell line. The cells were grown in incubators at 37° C. with 5% CO₂. An aliquot of cells was used for detection of tumor antigen BCMA expression prior to use.

### In Vitro Cytokine Secretion Assay

Cytokine secretion of anti-BCMA CAR-T (referred to as effector cells) in response to a BCMA-expressing target cell was evaluated by incubating CAR-T cells with target cells at 2.5-fold E:T ratio for 20 h in 96-well flat-bottom plates. Effector cells were PI61, RIGS and BCMA10 CART cells generated using either the ARM or TM process. CART cells manufactured using the ARM process were plated for a 24 h washout condition to allow the cells to rest and minimize non-specific activity. Target cells include BCMA positive KMS11-luc or BCMA negative NALM6-luc. These target cells were added to the freshly plated T cells or T cells from the 24 h washout condition (ARM cells only). For this assay, the % transduction of CAR-T cells was normalized by addition of UTD to the BCMA CAR-Ts. This allowed for the comparison of the same number of CAR-Ts and same total T cell number in each sample. Supernatants from the 20-hour co-culture time point of effector to target were harvested from each well and frozen at −20° C. to be used for MSD cytokine analysis. The custom MSD V-PLEX Human IFN-γ, IL-2 Kit (#K151A0H-4A) was used to quantify the secreted cytokines in each of the supernatant samples.

### Results

### ARM Process Preserves T Cell Stemness

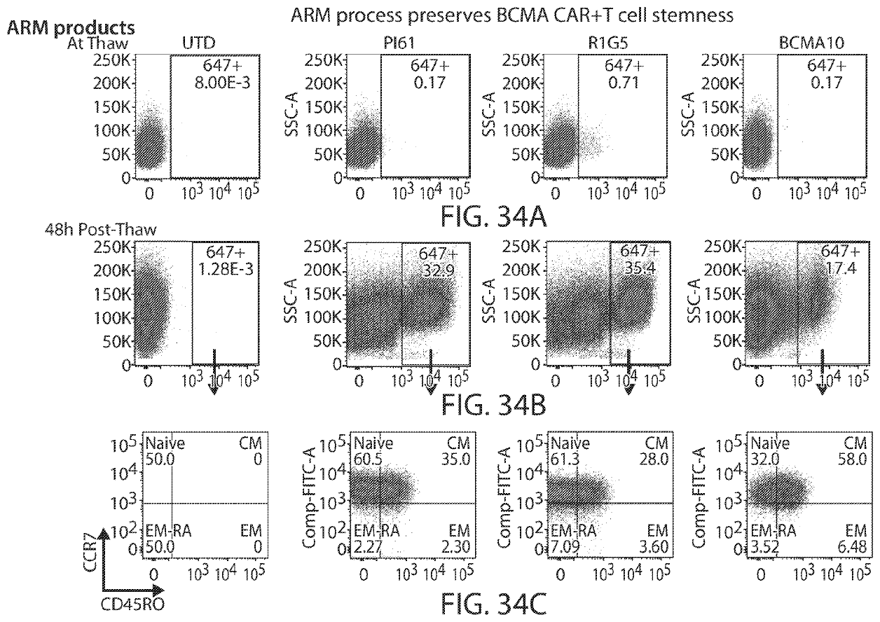

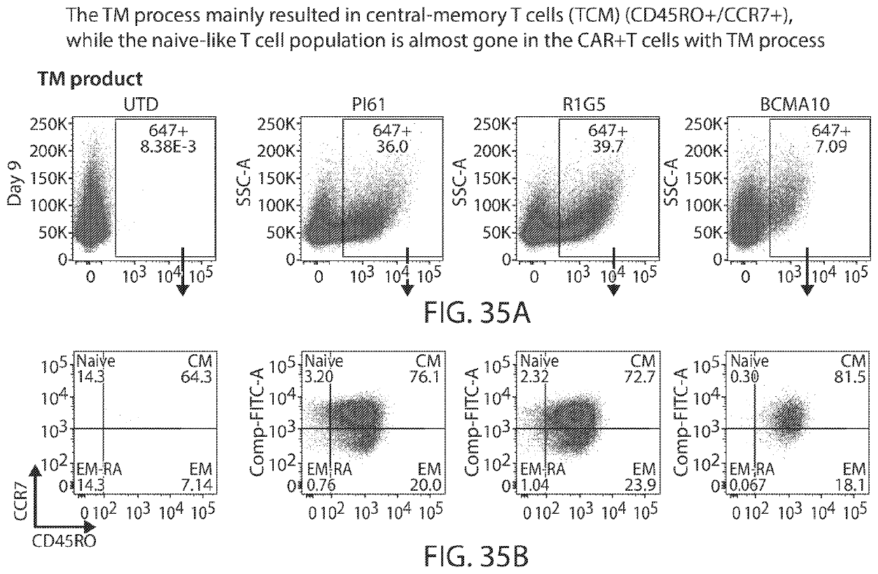

CAR-T cells generated using the ARM process were analyzed by flow cytometry to evaluate their CAR expression at thaw and 48 h post thaw, as well as the T-cell phenotype (FIGS. 34A, 34B, and 34C). For CAR-T cells manufactured using the TM process, CAR expression was assessed at day 9 before harvest (FIG. 35A). BCMA-CAR was almost undetectable at thaw shown in FIG. 34A. However, at 48 h post-thaw, BCMA-CAR was clearly being expressed as 32.9% for PI61, 35.9% for RIGS and 17.4% for BCMA10. The day 9 cells generated using the TM process show BCMA-CAR expression to be 36% for PI61, 40% for RIGS and 7% for BCMA10 (FIG. 35A). Analysis of the CAR+T-cell phenotype revealed that the ARM process retained naïve-like T cells (˜60% of CD45RO−/CCR7+ for PI61 and RIGS, 32% of CD45RO−/CCR7+ for BCMA10) (FIG. 34C). The TM process mainly resulted in central-memory T cells (TCM) (72˜81% CD45RO+/CCR7+ for all three BCMA CAR-Ts), while the naïve-like T cell population was almost gone in the CAR+T cells manufactured using the TM process (FIG. 35B). Overall, the naïve T-cell population largely overlaps with CD45RO−/CD27+ Tstem cells described by previous reports (Cohen A D, et al (2019). J Clin Invest. 130. pii: 126397. doi: 10.1172/JCI126397; Fraietta, J A, et al (2018). Nat Med, 24(5); 563-571) and is associated with responses and CAR-T expansion.

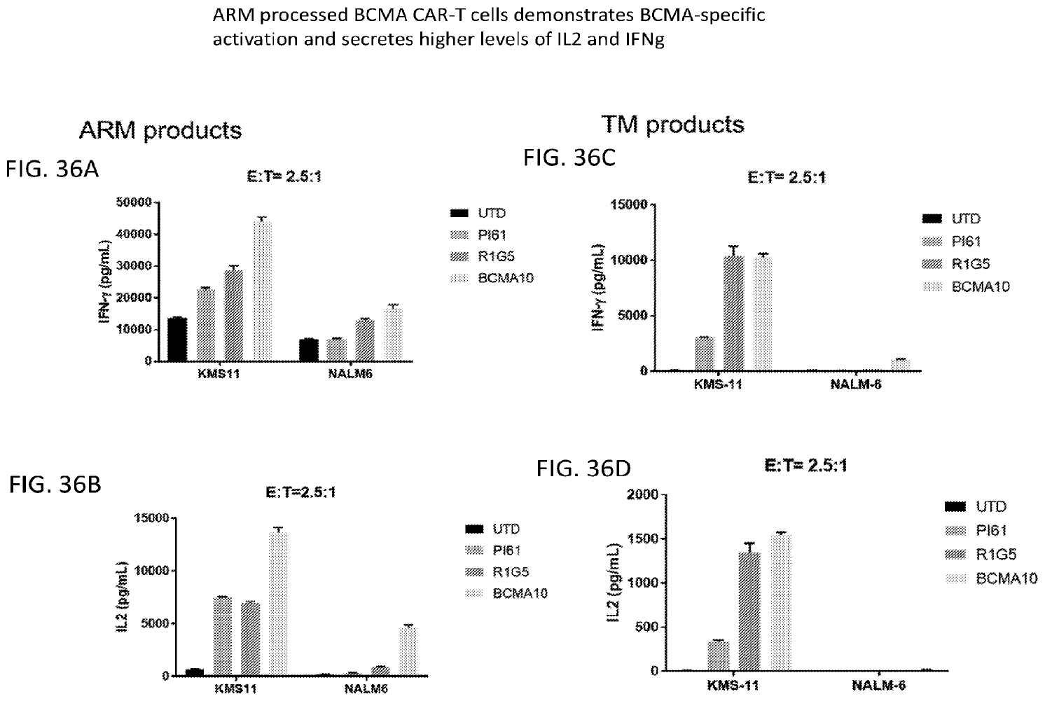

In addition to its phenotype, the final PI61, RIGS and BCMA10 CART cell products were also assessed for their function in vitro. PI61, RIGS and BCMA10 cell products were thawed and co-cultured with the BCMA-expressing cell line KMS-11 at 1:1 ratio. Post-thaw ARM processed cells were rested for 24 h prior to co-culture being established. Comparing cytokine levels in the supernatants 24 hours after co-culture revealed a ˜5 to 25-fold increase of IL-2 and a ˜3 to 7-fold increase in levels of IFN-γ secreted by ARM products as compared to TM products as shown in FIGS. 36A-36D. Experiments with untransduced (UTD) cells that underwent the ARM or TM process confirmed BCMA-specific recognition by PI61, RIGS and BCMA10.

In summary, PI61, RIGS and BCMA10 CART cells produced using the ARM process demonstrate BCMA-specific activation in vitro and secretes higher levels of IL-2 and IFN-γ as compared to TM processed products, which correlates with the Tstem phenotype of CART cells produced using the ARM process.

### Example 10

### Gene Signature Analysis of CART Cells Manufactured Using the ARM Process

### Methods

### Single Cell RNAseq

Single cell RNAseq libraries were generated using the 10×Genomics Chromium Controller instrument and supporting library construction kits.

Cryopreserved cells were thawed, counted and flow sorted (if required for study question), prior to being loaded on a 10×Genomics Instrument. Individual cells were loaded into droplets and RNA within individual droplets was barcoded via a GemCode bead. Barcoded RNA was released from droplets and converted into a whole transcriptome Illumina compatible sequencing library.

Generated libraries were sequenced on an Illumina HiSeq Instrument and analyzed using 10×Genomics analysis pipeline and Loupe Cell Browser software.

### Single Cell Immune Cell Profiling

Whole transcriptome 10× Genomics single cell libraries were used as a template material to generate immune cell profiling and repertoire analysis. T cell receptor sequences were PCR amplified from Chromium Single Cell 5′ Libraries and analyzed on an Illumina sequencing instrument.

### Analysis Pipeline

Single cell RNAseq data was processed through the Cell Ranger analysis pipeline starting with FASTQ files. A detailed description of the Cell Ranger analysis pipeline can be found at: https://support.10×genomics.com/single-cell-gene-expression/software/pipelines/latest/what-is-cell-ranger. The general pipeline included alignment, filtering, barcode counting, and UMI counting. Cellular barcodes were used to generate gene-barcode matrices, determine clusters, and perform gene expression analysis. Gene expression count data was normalized using the Seurat Bioconductor package. Cells were discarded from the analysis that had less than 200 expressed genes. Genes were discarded from the analysis that were only expressed in 2 cells or less. The remaining data was normalized with the Seurat log normalization method using a scale factor of 10,000. Data was scaled by regressing on the number of detected molecules per cell. The gene set score (GeneSetScore) was calculated by taking the mean log normalized gene expression value of all the genes in the gene set.

Each gene is z-score normalized so that the mean expression of the gene across samples is 0 and standard deviation is 1. The gene set score is then calculated as the mean of the normalized values of the genes in the gene set. An exemplary gene set score calculation is described below.

For this example of gene set score calculation, the normalized gene expression of two (2) samples for six (6) genes is provided in Table 23. For the purposes of this exemplary calculation, the gene set consists of genes 1-4. Therefore, Sample 1 and 2 both have gene set scores of 0.

TABLE 23
Exemplary dataset for gene set score calculation

| | Sample 1 | Sample 2 |
|---|---|---|
| Gene 1 | −3 | 0 |
| Gene 2 | 3 | 0 |
| Gene 3 | 1 | 0 |
| Gene 4 | −1 | 0 |
| Gene 5 | 10 | 4 |
| Gene 6 | −5 | 3 |

The gene set “Up TEM vs. Down TSCM” includes the following genes: MXRA7, CLIC1, NAT13, TBC1D2B, GLCCI1, DUSP10, APOBEC3D, CACNB3, ANXA2P2, TPRG1, EOMES, MATK, ARHGAP10, ADAMS, MAN1A1, SLFN12L, SH2D2A, EIF2C4, CD58, MY01F, RAB27B, ERN1, NPC1, NBEAL2, APOBEC3G, SYTL2, SLC4A4, PIK3AP1, PTGDR, MAF, PLEKHA5, ADRB2, PLXND1, GNAO1, THBS1, PPP2R2B, CYTH3, KLRF1, FLJ16686, AUTS2, PTPRM, GNLY, and GFPT2.

The gene set “Up Treg vs. Down Teff” includes the following genes: C12orf75, SELPLG, SWAP70, RGS1, PRR11, SPATS2L, SPATS2L, TSHR, C14orf145, CASP8, SYT11, ACTN4, ANXA5, GLRX, HLA-DMB, PMCH, RAB11FIP1, IL32, FAM160B1, SHMT2, FRMD4B, CCR3, TNFRSF13B, NTNG2, CLDND1, BARD1, FCER1G, TYMS, ATP1B1, GJB6, FGL2, TK1, SLC2A8, CDKN2A, SKAP2, GPR55, CDCA7, S100A4, GDPDS, PMAIP1, ACOT9, CEP55, SGMS1, ADPRH, AKAP2, HDAC9, IKZF4, CARD17, VAV3, OBFC2A, ITGB1, CIITA, SETD7, HLA-DMA, CCR10, KIAA0101, SLC14A1, PTTG3P, DUSP10, FAM164A, PYHIN1, MYO1F, SLC1A4, MYBL2, PTTG1, RRM2, TP53INP1, CCR5, ST8SIA6, TOX, BFSP2, ITPRIPL1, NCAPH, HLA-DPB2, SYT4, NINJ2, FAM46C, CCR4, GBPS, C15orf53, LMCD1, MKI67, NUSAP1, PDE4A, E2F2, CD58, ARHGEF12, LOC100188949, FAS, HLA-DPB1, SELP, WEE1, HLA-DPA1, FCRL1, ICA1, CNTNAP1, OAS1, METTL7A, CCR6, HLA-DRB4, ANXA2P3, STAM, HLA-DQB2, LGALS1, ANXA2, PI16, DUSP4, LAYN, ANXA2P2, PTPLA, ANXA2P1, ZNF365, LAIR2, L00541471, RASGRP4, BCAS1, UTS2, MIAT, PRDM1, SEMA3G, FAM129A, HPGD, NCF4, LGALS3, CEACAM4, JAKMIP1, TIGIT, HLA-DRA, IKZF2, HLA-DRB1, FANK1, RTKN2, TRIB1, FCRL3, and FOXP3.

The gene set “Down sternness” includes the following genes: ACE, BATF, CDK6, CHD2, ERCC2, HOXB4, MEOX1, SFRP1, SP7, SRF, TAL1, and XRCCS.

The gene set “Up hypoxia” includes the following genes: ABCB1, ACAT1, ADM, ADORA2B, AK2, AK3, ALDH1A1, ALDH1A3, ALDOA, ALDOC, ANGPT2, ANGPTL4, ANXA1, ANXA2, ANXA5, ARHGAP5, ARSE, ART1, BACE2, BATF3, BCL2L1, BCL2L2, BHLHE40, BHLHE41, BIK, BIRC2, BNIP3, BNIP3L, BPI, BTG1, C11orf2, C7orf68, CA12, CA9, CALD1, CCNG2, CCT6A, CD99, CDK1, CDKN1A, CDKN1B, CITED2, CLK1, CNOT7, COL4A5, COL5A1, COL5A2, COL5A3, CP, CTSD, CXCR4, D4S234E, DDIT3, DDIT4, 1-Dec, DKC1, DR1, EDN1, EDN2, EFNA1, EGF, EGR1, EIF4A3, ELF3, ELL2, ENG, ENO1, ENO3, ENPEP, EPO, ERRFI1, ETS1, F3, FABP5, FGF3, FKBP4, FLT1, FN1, FOS, FTL, GAPDH, GBE1, GLRX, GPI, GPRCSA, HAP1, HBP1, HDAC1, HDAC9, HERC3, HERPUD1, HGF, HIF1A, HK1, HK2, HLA-DQB1, HMOX1, HMOX2, HSPA5, HSPD1, HSPH1, HYOU1, ICAM1, ID2, IFI27, IGF2, IGFBP1, IGFBP2, IGFBP3, IGFBP5, IL6, IL8, INSIG1, IRF6, ITGA5, JUN, KDR, KRT14, KRT18, KRT19, LDHA, LDHB, LEP, LGALS1, LONP1, LOX, LRP1, MAP4, MET, MIF, MMP13, MMP2, MMPI, MPI, MT1L, MTL3P, MUC1, MXI1, NDRG1, NFIL3, NFKB1, NFKB2, NOS1, NOS2, NOS2P1, NOS2P2, NOS3, NR3C1, NR4A1, NT5E, ODC1, P4HA1, P4HA2, PAICS, PDGFB, PDK3, PFKFB1, PFKFB3, PFKFB4, PFKL, PGAM1, PGF, PGK1, PGK2, PGM1, PIM1, PIM2, PKM2, PLAU, PLAUR, PLIN2, PLOD2, PNN, PNP, POLM, PPARA, PPAT, PROK1, PSMA3, PSMD9, PTGS1, PTGS2, QSOX1, RBPJ, RELA, RIOK3, RNASEL, RPL36A, RRP9, SAT1, SERPINB2, SERPINE1, SGSM2, SIAH2, SIN3A, SIRPA, SLC16A1, SLC16A2, SLC20A1, SLC2A1, SLC2A3, SLC3A2, SLC6A10P, SLC6A16, SLC6A6, SLC6A8, SORL1, SPP1, SRSF6, SSSCA1, STC2, STRA13, SYT7, TBPL1, TCEAL1, TEK, TF, TFF3, TFRC, TGFA, TGFB1, TGFB3, TGFBI, TGM2, TH, THBS1, THBS2, TIMM17A, TNFAIP3, TP53, TPBG, TPD52, TPI1, TXN, TXNIP, UMPS, VEGFA, VEGFB, VEGFC, VIM, VPS11, and XRCC6.

The gene set “Up autophagy” includes the following genes: ABL1, ACBD5, ACIN1, ACTRT1, ADAMTS7, AKR1E2, ALKBHS, ALPK1, AMBRA1, ANXA5, ANXA7, ARSB, ASB2, ATG10, ATG12, ATG13, ATG14, ATG16L1, ATG16L2, ATG2A, ATG2B, ATG3, ATG4A, ATG4B, ATG4C, ATG4D, ATG5, ATG7, ATG9A, ATG9B, ATP13A2, ATP1B1, ATPAF1-AS1, ATPIF1, BECN1, BECN1P1, BLOC1S1, BMP2KL, BNIP1, BNIP3, BOC, C11orf2, C11orf41, C12orf44, C12orf5, C14orf133, C1orf210, C5, C6orf106, C7orf59, C7orf68, C8orf59, C9orf72, CA7, CALCB, CALCOCO2, CAPS, CCDC36, CD163L1, CD93, CDC37, CDKN2A, CHAF1B, CHMP2A, CHMP2B, CHMP3, CHMP4A, CHMP4B, CHMP4C, CHMP6, CHST3, CISD2, CLDN7, CLEC16A, CLN3, CLVS1, COX8A, CPA3, CRNKL1, CSPG5, CTSA, CTSB, CTSD, CXCR7, DAP, DKKL1, DNAAF2, DPF3, DRAM1, DRAM2, DYNLL1, DYNLL2, DZANK1, EI24, EIF2S1, EPG5, EPM2A, FABP1, FAM125A, FAM131B, FAM134B, FAM13B, FAM176A, FAM176B, FAM48A, FANCC, FANCF, FANCL, FBX07, FCGR3B, FGF14, FGF7, FGFBP1, FIS1, FNBP1L, FOX01, FUNDC1, FUNDC2, FXR2, GABARAP, GABARAPL1, GABARAPL2, GABARAPL3, GABRA5, GDF5, GMIP, HAP1, HAPLN1, HBXIP, HCAR1, HDAC6, HGS, HIST1H3A, HIST1H3B, HIST1H3C, HIST1H3D, HIST1H3E, HIST1H3F, HIST1H3G, HIST1H3H, HIST1H3B, HIST1H3J, HK2, HMGB1, HPR, HSF2BP, HSP90AA1, HSPA8, IFI16, IPPK, IRGM, IST1, ITGB4, ITPKC, KCNK3, KCNQ1, KIAA0226, KIAA1324, KRCC1, KRT15, KRT73, LAMP1, LAMP2, LAMTOR1, LAMTOR2, LAMTOR3, LARP1B, LENG9, LGALS8, LIX1, LIX1L, LMCD1, LRRK2, LRSAM1, LSM4, MAP1A, MAP1LC3A, MAP1LC3B, MAP1LC3B2, MAP1LC3C, MAP1S, MAP2K1, MAP3K12, MARK2, MBDS, MDH1, MEX3C, MFN1, MFN2, MLST8, MRPS10, MRPS2, MSTN, MTERFD1, MTMR14, MTMR3, MTOR, MTSS1, MYH11, MYLK, MYOM1, NBR1, NDUFB9, NEFM, NHLRC1, NME2, NPC1, NR2C2, NRBF2, NTHL1, NUP93, OBSCN, OPTN, P2RX5, PACS2, PARK2, PARK7, PDK1, PDK4, PEX13, PEX3, PFKP, PGK2, PHF23, PHYHIP, PI4K2A, PIK3C3, PIK3CA, PIK3CB, PIK3R4, PINK', PLEKHM1, PLOD2, PNPO, PPARGC1A, PPY, PRKAA1, PRKAA2, PRKAB1, PRKAB2, PRKAG1, PRKAG2, PRKAG3, PRKD2, PRKG1, PSEN1, PTPN22, RAB12, RAB1A, RAB1B, RAB23, RAB24, RAB33B, RAB39, RAB7A, RB1CC1, RBM18, REEP2, REP15, RFWD3, RGS19, RHEB, RIMS3, RNF185, RNF41, RPS27A, RPTOR, RRAGA, RRAGB, RRAGC, RRAGD, S100A8, S100A9, SCN1A, SERPINB10, SESN2, SFRP4, SH3GLB1, SIRT2, SLC1A3, SLC1A4, SLC22A3, SLC25A19, SLC35B3, SLC35C1, SLC37A4, SLC6A1, SLCO1A2, SMURF1, SNAP29, SNAPIN, SNF8, SNRPB, SNRPB2, SNRPD1, SNRPF, SNTG1, SNX14, SPATA18, SQSTM1, SRPX, STAM, STAM2, STAT2, STBD1, STK11, STK32A, STOM, STX12, STX17, SUPT3H, TBC1D17, TBC1D25, TBC1D5, TCIRG1, TEAD4, TECPR1, TECPR2, TFEB, TM9SF1, TMBIM6, TMEM203, TMEM208, TMEM39A, TMEM39B, TMEM59, TMEM74, TMEM93, TNIK, TOLLIP, TOMM20, TOMM22, TOMM40, TOMM5, TOMM6, TOMM7, TOMM70A, TP53INP1, TP53INP2, TRAPPC8, TREM1, TRIM17, TRIMS, TSG101, TXLNA, UBA52, UBB, UBC, UBQLN1, UBQLN2, UBQLN4, ULK1, ULK2, ULK3, USP10, USP13, USP30, UVRAG, VAMP7, VAMP5, VDAC1, VMP1, VPS11, VPS16, VPS18, VPS25, VPS28, VPS33A, VPS33B, VPS36, VPS37A, VPS37B, VPS37C, VPS37D, VPS39, VPS41, VPS4A, VPS4B, VTA1, VTI1A, VTI1B, WDFY3, WDR45, WDR45L, WIPI1, WIPI2, XBP1, YIPF1, ZCCHC17, ZFYVE1, ZKSCAN3, ZNF189, ZNF593, and ZNF681.

The gene set “Up resting vs. Down activated” includes the following genes: ABCA7, ABCF3, ACAP2, AMT, ANKH, ATF7IP2, ATG14, ATP1A1, ATXN7, ATXN7L3B, BCL7A, BEX4, BSDC1, BTG1, BTG2, BTN3A1, C11orf21, C19orf22, C21orf2, CAMK2G, CARS2, CCNL2, CD248, CD5, CD55, CEP164, CHKB, CLK1, CLK4, CTSL1, DBP, DCUN1D2, DENND1C, DGKD, DLG1, DUSP1, EAPP, ECE1, ECHDC2, ERBB2IP, FAM117A, FAM134B, FAM134C, FAM169A, FAM190B, FAU, FLJ10038, FOXJ2, FOXJ3, FOXL1, FOXO1, FXYD5, FYB, HLA-E, HSPA1L, HYAL2, ICAM2, IFIT5, IFITM1, IKBKB, IQSEC1, IRS4, KIAA0664L3, KIAA0748, KLF3, KLF9, KRT18, LEF1, LINC00342, LIPA, LIPT1, LLGL2, LMBR1L, LPAR2, LTBP3, LYPD3, LZTFL1, MANBA, MAP2K6, MAP3K1, MARCH8, MAU2, MGEA5, MMP8, MPO, MSL1, MSL3, MYH3, MYLIP, NAGPA, NDST2, NISCH, NKTR, NLRP1, NOSIP, NPIP, NUMA1, PAIP2B, PAPD7, PBXIP1, PCIF1, PI4KA, PLCL2, PLEKHA1, PLEKHF2, PNISR, PPFIBP2, PRKCA, PRKCZ, PRKD3, PRMT2, PTP4A3, PXN, RASA2, RASA3, RASGRP2, RBM38, REPIN1, RNF38, RNF44, ROR1, RPL30, RPL32, RPLP1, RPS20, RPS24, RPS27, RPS6, RPS9, RXRA, RYK, SCAND2, SEMA4C, SETD1B, SETD6, SETX, SF3B1, SH2B1, SLC2A4RG, SLC35E2B, SLC46A3, SMAGP, SMARCE1, SMPD1, SNPH, SP140L, SPATA6, SPG7, SREK1IP1, SRSF5, STAT5B, SVIL, SYF2, SYNJ2BP, TAF1C, TBC1D4, TCF20, TECTA, TES, TMEM127, TMEM159, TMEM30B, TMEM66, TMEM8B, TP53TG1, TPCN1, TRIM22, TRIM44, TSC1, TSC22D1, TSC22D3, TSPYL2, TTC9, TTN, UBE2G2, USP33, USP34, VAMP1, VILL, VIPR1, VPS13C, ZBED5, ZBTB25, ZBTB40, ZC3H3, ZFP161, ZFP36L1, ZFP36L2, ZHX2, ZMYM5, ZNF136, ZNF148, ZNF318, ZNF350, ZNF512B, ZNF609, ZNF652, ZNF83, ZNF862, and ZNF91.

The gene set “Progressively up in memory differentiation” includes the following genes: MTCH2, RAB6C, KIAA0195, SETD2, C2orf24, NRD1, GNA13, COPA, SELT, TNIP1, CBFA2T2, LRP10, PRKCI, BRE, ANKS1A, PNPLA6, ARL6IP1, WDFY1, MAPK1, GPR153, SHKBP1, MAP1LC3B2, PIP4K2A, HCN3, GTPBP1, TLN1, C4orf34, KIF3B, TCIRG1, PPP3CA, ATG4D, TYMP, TRAF6, C17orf76, WIPF1, FAM108A1, MYL6, NRM, SPCS2, GGT3P, GALK1, CLIP4, ARL4C, YWHAQ, LPCAT4, ATG2A, IDS, TBC1D5, DMPK, ST6GALNAC6, REEP5, ABHD6, KIAA0247, EMB, TSEN54, SPIRE2, PIWIL4, ZSCAN22, ICAM1, CHD9, LPIN2, SETD8, ZC3H12A, ULBP3, IL15RA, HLA-DQB2, LCP1, CHP, RUNX3, TMEM43, REEP4, MEF2D, ABL1, TMEM39A, PCBP4, PLCD1, CHST12, RASGRP1, C1orf58, C11orf63, C6orf129, FHOD1, DKFZp434F142, PIK3CG, ITPR3, BTG3, C4orf50, CNNM3, IFI16, AK1, CDK2AP1, REL, BCL2L1, MVD, TTC39C, PLEKHA2, FKBP11, EML4, FANCA, CDCA4, FUCA2, MFSD10, TBCD, CAPN2, IQGAP1, CHST11, PIK3R1, MYO5A, KIR2DL3, DLG3, MXD4, RALGDS, S1PR5, WSB2, CCR3, TIPARP, SP140, CD151, SOX13, KRTAPS-2, NF1, PEA15, PARP8, RNF166, UEVLD, LIMK1, CACNB1, TMX4, SLC6A6, LBA1, SV2A, LLGL2, IRF1, PPP2R5C, CD99, RAPGEF1, PPP4R1, OSBPL7, FOXP4, SLA2, TBC1D2B, ST7, JAZF1, GGA2, PI4K2A, CD68, LPGAT1, STX11, ZAK, FAM160B1, RORA, C8orf80, APOBEC3F, TGFBI, DNAJC1, GPR114, LRP8, CD69, CMIP, NAT13, TGFB1, FLJ00049, ANTXR2, NR4A3, IL12RB1, NTNG2, RDX, MLLT4, GPRIN3, ADCY9, CD300A, SCD5, ABI3, PTPN22, LGALS1, SYTL3, BMPR1A, TBK1, PMAIP1, RASGEF1A, GCNT1, GABARAPL1, STOM, CALHM2, ABCA2, PPP1R16B, SYNE2, PAM, C12orf75, CLCF1, MXRA7, APOBEC3C, CLSTN3, ACOT9, HIP1, LAG3, TNFAIP3, DCBLD1, KLF6, CACNB3, RNF19A, RAB27A, FADS3, DLG5, APOBEC3D, TNFRSF1B, ACTN4, TBKBP1, ATXN1, ARAP2, ARHGEF12, FAM53B, MAN1A1, FAM38A, PLXNC1, GRLF1, SRGN, HLA-DRB5, B4GALT5, WIPI1, PTPRJ, SLFN11, DUSP2, ANXA5, AHNAK, NEO1, CLIC1, EIF2C4, MAP3K5, IL2RB, PLEKHG1, MYO6, GTDC1, EDARADD, GALM, TARP, ADAMS, MSC, HNRPLL, SYT11, ATP2B4, NHSL2, MATK, ARHGAP18, SLFN12L, SPATS2L, RAB27B, PIK3R3, TP53INP1, MBOAT1, GYG1, KATNAL1, FAM46C, ZC3HAV1L, ANXA2P2, CTNNA1, NPC1, C3AR1, CRIM1, SH2D2A, ERN1, YPEL1, TBX21, SLC1A4, FASLG, PHACTR2, GALNT3, ADRB2, PIK3AP1, TLR3, PLEKHA5, DUSP10, GNAO1, PTGDR, FRMD4B, ANXA2, EOMES, CADM1, MAF, TPRG1, NBEAL2, PPP2R2B, PELO, SLC4A4, KLRF1, FOSL2, RGS2, TGFBR3, PRF1, MYO1F, GAB3, C17orf66, MICAL2, CYTH3, TOX, HLA-DRA, SYNE1, WEE1, PYHIN1, F2R, PLD1, THBS1, CD58, FAS, NETO2, CXCR6, ST6GALNAC2, DUSP4, AUTS2, C1orf21, KLRG1, TNIP3, GZMA, PRR5L, PRDM1, ST8SIA6, PLXND1, PTPRM, GFPT2, MYBL1, SLAMF7, FLJ16686, GNLY, ZEB2, CST7, IL18RAP, CCL5, KLRD1, and KLRB1.

The gene set “Up TEM vs. Down TN” includes the following genes: MYO5A, MXD4, STK3, S1PR5, GLCCI1, CCR3, SOX13, KRTAP5-2, PEA15, PARP8, RNF166, UEVLD, LIMK1, SLC6A6, SV2A, KPNA2, OSBPL7, ST7, GGA2, PI4K2A, CD68, ZAK, RORA, TGFBI, DNAJC1, JOSD1, ZFYVE28, LRP8, OSBPL3, CMIP, NAT13, TGFB1, ANTXR2, NR4A3, RDX, ADCY9, CHN1, CD300A, SCD5, PTPN22, LGALS1, RASGEF1A, GCNT1, GLUL, ABCA2, CLDND1, PAM, CLCF1, MXRA7, CLSTN3, ACOT9, METRNL, BMPR1A, LRIG1, APOBEC3G, CACNB3, RNF19A, RAB27A, FADS3, ACTN4, TBKBP1, FAM53B, MAN1A1, FAM38A, GRLF1, B4GALT5, WIPI1, DUSP2, ANXA5, AHNAK, CLIC1, MAP3K5, ST8SIA1, TARP, ADAMS, MATK, SLFN12L, PIK3R3, FAM46C, ANXA2P2, CTNNA1, NPC1, SH2D2A, ERN1, YPEL1, TBX21, STOM, PHACTR2, GBP5, ADRB2, PIK3AP1, DUSP10, PTGDR, EOMES, MAF, TPRG1, NBEAL2, NCAPH, SLC4A4, FOSL2, RGS2, TGFBR3, MYO1F, C17orf66, CYTH3, WEE1, PYHIN1, F2R, THBS1, CD58, AUTS2, FAM129A, TNIP3, GZMA, PRR5L, PRDM1, PLXND1, PTPRM, GFPT2, MYBL1, SLAMF7, ZEB2, CST7, CCL5, GZMK, and KLRB1.

Other gene sets describing similar processes and/or characteristics can also be used to characterize cell phenotypes described above.

Cell Ranger VDJ was used to generate single cell VDJ sequences and annotations for each single cell 5′ library. Loupe Cell Browser software and Bioconductor packages were used for data analysis and visualization.

### Results

This example aims to compare T cell states between purified T cells which served as input cells, CART cells manufactured using the ARM process (labeled as “Day 1” cells), and CART cells manufactured using the TM process (labeled as “Day 9” cells) using single-cell RNA-seq (scRNA-seq). In addition, single-cell TCR-seq (scTCR-seq) was performed to study clonality and track cell differentiation from input to post-manufacturing materials.

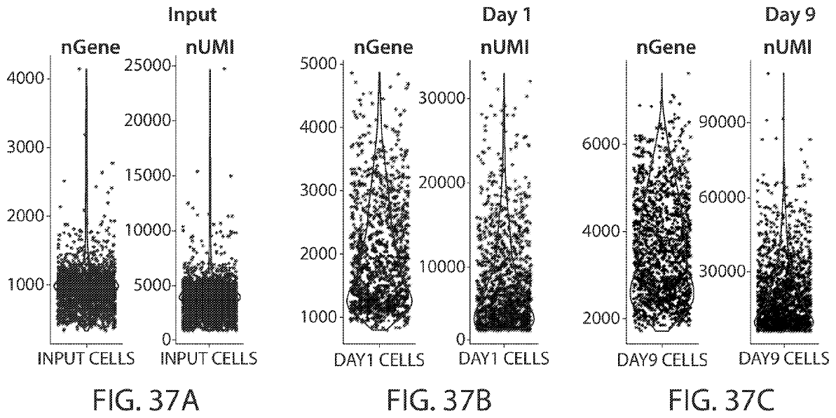

As shown in FIGS. 37A-37C, input cells had the fewest expressed genes and UMIs, suggesting these cells were not transcriptionally active and were in a resting state. Day 1 and Day 9 cells were expressing more genes, with Day 9 cells being the most transcriptionally active. Similar results are shown in FIGS. 38A-38D. Input cells were not expressing proliferation genes (FIGS. 38A and 38D).

Additional gene set analysis data are shown in FIGS. 39A-39E. Different populations of cells were compared using the median gene set scores. Day 1 cells and input cells were in a younger, more stem-like memory state (FIGS. 39A-39C). In FIG. 39A, the median GeneSetScore (Up TEM vs. Down TSCM) values for Day 1 cells, Day 9 cells, and input cells are −0.084, 0.035, and −0.1, respectively. In FIG. 39B, the median GeneSetScore (Up Treg vs. Down Teff) values for Day 1 cells, Day 9 cells, and input cells are −0.082, 0.087, and −0.071, respectively. In FIG. 39C, the median GeneSetScore (Down stemness) values for Day 1 cells, Day 9 cells, and input cells are −0.062, 0.14, and −0.081, respectively.

In addition, Day 1 cells were in a more ideal metabolic state compared to Day 9 cells (FIGS. 39D and 39E). In FIG. 39D, the median GeneSetScore (Up hypoxia) values for Day 1 cells, Day 9 cells, and input cells are 0.019, 0.11, and −0.096, respectively. In FIG. 39E, the median GeneSetScore (Up autophagy) values for Day 1 cells, Day 9 cells, and input cells are 0.066, 0.11, and −0.09, respectively.

Based on gene expression, the input cells contain four clusters. Cluster 0 is characterized by high expression of LMNA, S100A4, etc. Cluster 1 is characterized by high expression of RP913, PRKCQ-AS1, etc. Cluster 2 is characterized by high expression of PR11-291B21.2, CD8B, etc. Cluster 3 is characterized by high expression of NKG7, GZMH, CCL5, CST7, GNLY, FGFBP2, GZMA, CCL4, CTSW, CD8A, etc. In a T-Distributed Stochastic Neighbor Embedding (TSNE) plot for the input cells, Cluster 3 stood out from the other cells, and Cluster 1 and Cluster 2 were hard to differentiate.

According to the gene set analysis shown in FIGS. 40A-40C, Cluster 0 and Cluster 3 were enriched for a T regulatory phenotype compared to Cluster 1 and Cluster 2 which were enriched for a T effector phenotype. Cluster 3 was dominated by late memory/effector memory (TEM) cells, Cluster 1 and Cluster 2 were early memory and naïve cells, and Cluster 0 is in the middle. The majority of the input cells were in an early memory, naïve state. Without wishing to be bound by theory, these cells may do the best during the manufacturing procedure.

Less transcriptional heterogeneity was seen in Day 1 cells and Day 9 cells (data not shown).

Like the input population, Day 1 cells showed a large cluster of early memory cells and a smaller cluster of late memory cells in a TSNE plot. similar to what was seen with Cluster 3 of the input cells. In contrast, Day 9 cells did not show distinct clusters of early memory cells in a TSNE plot. This implies that by day 9, the cells had become more homogeneous.

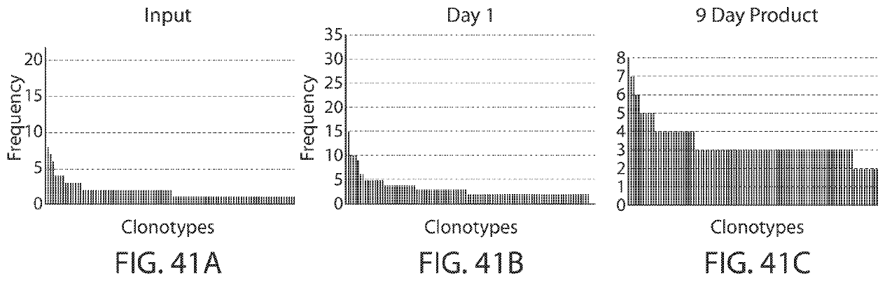

TCRs were sequenced and clonotype diversity was measured. Overall, the three clonotype profiles were very flat—most clones were only picked up once (FIGS. 41A-41C and Table 24). Shannon entropy in Table 24 measures the flatness of the distribution. The dominant clones in the input cells were late memory cells. Day 1 cells looked similar to the input cells but started to even out. By day 9, the dominate clones had substantially evened out and the distribution was much more flat. The diversity measurement was the highest at day 9 because there was a much more even and flat distribution in Day 9 cells than in the input cells or Day 1 cells.

TABLE 24
Measurements of TCR diversity

| | | Day 1 | Day 9 |
|---|---|---|---|
| | Input | product | product |
| Average clones per clonotype | 1.10 | 1.05 | 1.07 |
| Estimated number of cells | 7344 | 7687 | 7233 |
| Total number of clonotypes | 5325 | 7403 | 6736 |
| Diversity | 342.27 | 802.94 | 3382.62 |
| Normalized Shannon entropy | 9.98E−01 | 9.95E−01 | 9.96E−01 |

### Summary

There were significant T cell state differences between Day 1 and Day 9 products. Day 1 cells were much more similar to input cells and had enrichment for stemness signatures, indicating a more efficacious product.

### Example 11

### Phase I, Open Label, Study of B-Cell Maturation Antigen (BCMA)-Directed CAR-T Cells in Adult Patients with Relapsed and/or Refractory Multiple Myeloma (MM)

This study evaluates the safety and tolerability of anti-BCMA CART-T cell therapy in adult MM subjects who are relapsed and/or refractory to at least two prior treatment regimens, including an IMiD (e.g. lenalidomide or pomalidomide), a proteasome inhibitor (e.g. bortezomib, carfilzomib), and an approved anti-CD38 antibody (e.g. daratumumab), if available, and have documented evidence of disease progression (IMWG criteria).

The anti-BCMA CAR comprises a PI61 anti-BCMA scFv, a CD8 hinge and transmembrane region, a 4-1BB costimulatory domain, and a CD3 zeta signaling domain. In this study, the anti-BCMA CAR-T cell products are manufactured using the Activated Rapid Manufacturing (ARM) process. Such cells are referred to as “ARM-BCMA CAR.” Specifically, T cells are enriched from a subject's leukapheresis unit using commercially available magnetic beads capturing CD4 and CD8 co-receptors on the T cell surface. Enriched T cells are then stimulated with a colloidal polymeric nanomatrix covalently attached to humanized recombinant agonist antibodies against human CD3 and CD28. Twenty-four hours after seeding, activation and transduction, CAR-T cells are harvested and washed to remove residual non-integrated vector and non-bound activating matrix. After the wash, BCMA CART cell therapy is concentrated and cryopreserved. Results from a release testing procedure are required prior to release of the product for administration.

Compared to the TM process for CAR-T cells, which relies on an ex vivo T-cell expansion period lasting 7-8 days after transduction with lentiviral vector, the ARM process does not include ex vivo T-cell expansion. In contrast, ARM produced T cells are harvested 24 hours after gene transfer, allowing them to expand in vivo in patients. The greater in vivo T cell expansion achieved with the ARM process is predicted to result in a less differentiated T cell phenotype, preserving a greater fraction of memory stem T cells in the final cell product. The presence of less differentiated, memory CAR-T cells has been associated with improved antitumor efficacy in clinical studies (Fraietta J A, et al., (2018) Nat Med, 24(5); 563-71). Without wishing to be bound by theory, BCMA CART cells comprised of a greater fraction of memory T stem cells result in enhanced CAR-T cell expansion in patients, thus overcoming effector T cell exhaustion and resulting in more durable efficacy in MM patients compared with BCMA CARTs produced under traditional manufacturing processes.

The ARM process produces CAR-T cells composed of a significantly greater proportion of naïve-like memory T cells (CCR7+/CD45RO−) in both the overall product and the CAR-positive fraction as compared to CART cells manufactured using the traditional manufacturing (TM) process. ARM-BCMA CAR has shown tumor eradication in preclinical MM models in a dose responsive fashion. ARM-BCMA CAR is at least five-fold more potent as compared to BCMA CAR-T cells generated with the TM process and led to extended CAR-T expansion in vivo, with higher levels of systemic cytokines. Together, these results support the hypothesis that anti-BCMA CAR-T cell products manufactured with the ARM process contain T cells with a pronounced memory stem cell phenotype, resulting in a BCMA CAR-T cell product with enhanced engraftment, expansion, and anti-MM properties.

In this phase I study, each subject is first evaluated for clinical eligibility during screening. Subjects eligible for inclusion in this study must meet all of the following criteria: (1) ≥18 years of age at the time of ICF signature; (2) ECOG performance status that is either 0 or 1 at screening; (3) subjects with MM who are relapsed and/or refractory to at least 2 prior treatment regimens, including an IMiD (e.g. lenalidomide or pomalidomide), a proteasome inhibitor (e.g. bortezomib, carfilzomib), and an approved anti-CD38 antibody (e.g. daratumumab) (if available) and have documented evidence of disease progression (IMWG criteria); (4) subjects must have measurable disease defined by at least 1 of the following 3 measurements: serum M-protein≥1.0 g/dL, urine M-protein≥200 mg/24 hours, or serum free light chain (sFLC)>100 mg/L of involved FLC; (5) All patients must be suitable for serial bone marrow biopsy and/or aspirate collection according to institution's guidelines and be willing to undergo this repeated procedure as described for this study; (6) subjects must meet the following hematological values at screening: absolute neutrophil count (ANC)≥1,000/mm³ (≥1×10⁹/L) without growth factor support within 7 days prior to testing, absolute number of CD3+ T cells>150/mm³ (>0.15 ×10⁹/L) without transfusion support within 7 days prior to testing, platelets≥50 000/mm³ (≥50×10⁹/L), and hemoglobin≥8.0 g/dl (≥4.9 mmol/L); (7) patient must be deemed suitable by investigator to undergo fludarabine/cyclophosphamide LD regimen; and (8) must have a leukapheresis material of non-mobilized cells accepted for manufacturing. If eligible, a subject has a leukapheresis product collected and submitted for CAR-T manufacture. The subject is enrolled with the acceptance of their leukapheresis product for the start of manufacture.

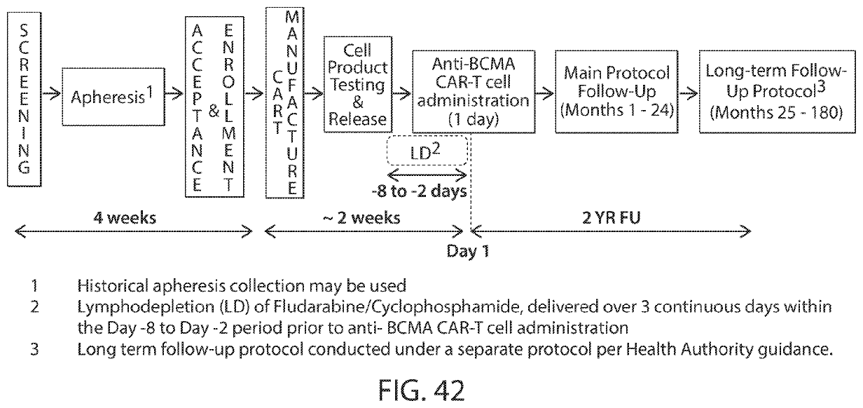

Subjects receive lymphodepletion (LD) chemotherapy only after the final product has been confirmed to be available. Following LD chemotherapy, a single dose of anti-BCMA CAR-T cell product is administered via an intravenous (i.v.) injection to a subject within 90 minutes from thawing (FIG. 42). The starting dose of ARM-BCMA CAR is 1 ×10⁷ viable CAR-positive T cells. The dose of 5 ×10⁷ viable CAR-positive T cells is also tested. Each subject is hospitalized for the first 72 hours following anti-BCMA CAR-T cell administration.

For pharmacokinetic analysis, serial blood samples are collected at different time points to measure ARM-BCMA CAR cellular kinetics in peripheral blood by flow cytometry and qPCR, in bone marrow by flow cytometry and qPCR, to measure cellular and humoral immunogenicity, and to measure potential pharmacodynamic markers including sBCMA, BAFF, and APRIL, in peripheral blood by ELISA. In particular, subjects are analyzed for the amount of CAR transgene in peripheral blood, bone marrow, or other relevant tissues; the surface expression of CAR-positive T cells in the peripheral blood or bone marrow; the anti-mCAR antibodies in the serum; the percentage of IFN-γ positive CD4/CD8 T cells in PBMC; markers of immune cell activation; soluble immune factors and cytokines (e.g., sBCMA, IFN-γ, IL-2, IL-4, IL-6, IL-8, IL-10, IL-15, TNF-α), CAR-T clonality; and the levels of soluble BCMA, APRIL, and BAFF in the serum.

### Example 12

### Manufacturing BCMA CART Cells Using the Activated Rapid Manufacturing (ARM) Process

The ARM process of BCMA CART cells initiates with the preparation of the media as outlined in Table 25.

Cryopreserved leukapheresis product is used as the starting material and is processed for T cell enrichment. When available, the apheresis paper work is utilized to define the T cell percentage. In the absence of the T cell percentage data on the apheresis paperwork, the sentinel vial testing is performed on incoming cryopreserved leukapheresis products to obtain T cell percentage target for the apheresis. The results for the T cell percentage determine how many bags are thawed on Day 0 of the ARM process.

TABLE 25
Media and Buffer type and point of
use during BCMA CART manufacturing

| Media Type | Source | Point of Use |
|---|---|---|
| CliniMACS ® Buffer/human | Prepared by | Day 0 Processing |
| serum albumin (HSA) (0.5% | operator on | on Cell Wash/ |
| in working concentration) | day 0 | Separator |
| Rapid Media | Prepared by | Day 0 for Cell |
| | operator on | Seeding |
| | day 0 | |
| PBS/HSA (1% or 2% in | Prepared by | Harvest and culture |
| working concentration) | operator on | Wash Media (Day 1) |
| | day 0 | |
| Cryostor10 (CS10) | Commercially | Harvest Formulation |
| | available | |

Cryopreserved leukapheresis is thawed, washed, and then undergoes T cell selection and enrichment using CliniMACS® microbead technology. Viable nucleated cells (VNCs) are activated with TransACT (Miltenyi) and transduced with a lentiviral vector encoding the CAR. The viable cells selected with the Miltenyi microbeads are seeded into the centricult on the Prodigy®, which is a non-humidified incubation chamber. While in culture, the cells are suspended in Rapid media, which is an OpTmizer™ CTS™ based medium that contains the CTS™ Supplement (ThermoFisher), Glutamax, IL-2 and 2% Immune cell serum replacement amongst its components to promote T cell activation and transduction. Lentiviral transduction is performed once on the day of seeding after the TransACT has been added to the diluted cells in the culture media. Lentiviral vector will be thawed immediately prior to use on day of seeding for up to 30 minutes at room temperature.

From the start of the process on Day 0 to the initiation of the culture wash and harvest, BCMA CART cells are cultured for 20-28 hours from seeding. Following culture, the cell suspension undergoes two culture washes and one harvest wash within the centricult chamber (Miltenyi Biotech).

After the harvest wash on the CliniMACS® Prodigy® on day 1, the cell suspension is sampled to determine viable cell count and viability. Cell suspension is then transferred to a centrifuge to be pelleted manually. The supernatant is removed, and the cell pellet is re-suspended in CS10 (BioLife Solution), resulting in a product formulation with a final DMSO concentration of ˜10.0%. The viable cell count is formulated at the end of harvest for dosing. The doses are then distributed into individual cryobags and analytical sampling into cryovials.

Cryopreserved products are stored in monitored LN2 storage tanks, in a secure, limited access area until final release and shipping.

### Example 13

### Characterization of BCMA CART Cells Manufactured Using the Activated Rapid Manufacturing (ARM) Process

### Summary

This example describes characterization of BCMA CART cells manufactured using the ARM process. The ARM process produces CAR-T cells composed of a significantly greater proportion of naïve-like memory T cells (CCR7+/CD45RO−) as compared to the traditional manufacturing (TM) product. In a preclinical model of multiple myeloma (MM), BCMA CART cells manufactured using the ARM process induced tumor regression in a dose-dependent manner and was up to 5-fold more efficacious in killing tumors compared to BCMA CART cells manufactured using the TM process. In addition, ARM-manufactured cells showed extended CART expansion in vivo (up to 3 folds higher Cmax and AUCO-21d) and induced higher systemic cytokines (IFN-γ by 3.5 folds) compared to TM-manufactured cells. Together, these results support the hypothesis that BCMA CART cells manufactured with the ARM process contain T cells with a pronounced memory stem cell phenotype and an enhanced in vivo expansion potential.

Using the ARM process, CAR could be stably expressed at 96 h after viral addition (also referred to as 72 h at post-thaw of the product). Therefore, 96 h post-viral addition or 72 h post-thaw is considered to be a surrogate time point for CAR expression for in vitro and in vivo activity. BCMA CART cells manufactured using the ARM process preserve a less differentiated cell population, and show higher target specific cytokine production in vitro, when compared to BCMA CART cells manufactured under the TM process.

BCMA CART cells manufactured using the ARM process demonstrated high specificity to BCMA using a commercial human plasma membrane protein array. The assay detected binding to BCMA (TNFRSF17) but no other strong, medium, or weak binders. The screen did not identify with any high confidence the presence of cross-reacting proteins of the anti-human BCMA single chain antibody variable fragment (scFv) (PI61) expressed in the BCMA CART product. Target distribution studies were performed to determine potential off-tumor on-target toxicity. Immunohistochemistry (IHC), in situ hybridization (ISH), and polymerase chain reaction (PCR) assays were utilized to examine the distribution of BCMA in normal human tissues. These analyses demonstrated that BCMA expression was limited to sites containing normal plasma cells (PCs), such as secondary lymphoid organs, bone marrow and mucosal associated lymphoid tissues. Because central nervous system (CNS) neurotoxicity has been a concern with other cell-based therapies, expression in brain was examined. No staining in the CNS was observed by immunohistochemistry using a commercially available antibody shown to be specific for BCMA nor by binding assays using a human-rabbit chimeric tool antibody containing a BCMA targeting scFv. These findings were confirmed by the absence of BCMA mRNA in these tissues as measured by in situ hybridization and PCR based splice variant analysis. BCMA CART targeting of normal PCs and BCMA-expressing plasmacytoid dendritic cells is likely to result in their depletion; however, targeting of other cell types is not anticipated.

### Results

The studies described below compared BCMA CART cells manufactured using the ARM process (referred to as “ARM-BCMA CAR”) with BCMA CART cells manufactured using the TM process (referred to as “TM-BCMA CAR” or “TM-BCMA CAR*”). The CAR expressed in ARM-BCMA CAR and the CAR expressed in TM-BCMA CAR* have the same sequence, comprising a PI61 scFv, a CD8 hinge and transmembrane region, a 4-1BB costimulatory domain, and a CD3 zeta signaling domain. The CAR expressed in TM-BCMA CAR comprises a BCMA10 scFv, a CD8 hinge and transmembrane region, a 4-1BB costimulatory domain, and a CD3 zeta signaling domain.

### ARM-BCMA CAR Expression Kinetics In Vitro

In contrast to TM which measures lentiviral integration of the CAR transgene after 8-9 days, in the ARM process, the lentiviral transgene may not be fully integrated and truly expressed within 24 h post lentiviral addition, as lentiviral pseudotransduction could occur (Haas D L, et al., (2000) Mol Ther; 2(1):71-80; Galla M, et al., (2004) Mol Cell; 16(2):309-15). Therefore, the BCMA-CAR expression pattern was evaluated over time by extended culturing of ARM-BCMA CAR in vitro in the presence or absence of 3′-azido-3′-deoxythymidine (AZT) to evaluate the potential pseudotransduction versus stable integration and expression of the CAR transgene. Flow cytometry (FACS) analyses were performed to detect CAR surface expression at 24 h, 48 h, 72 h, 96 h and 168 h post T cell activation and transduction with the lentiviral vector. In some cases, ARM-BCMA CAR and an aliquot of this product were frozen down immediately upon harvest for additional characterization in other assays.

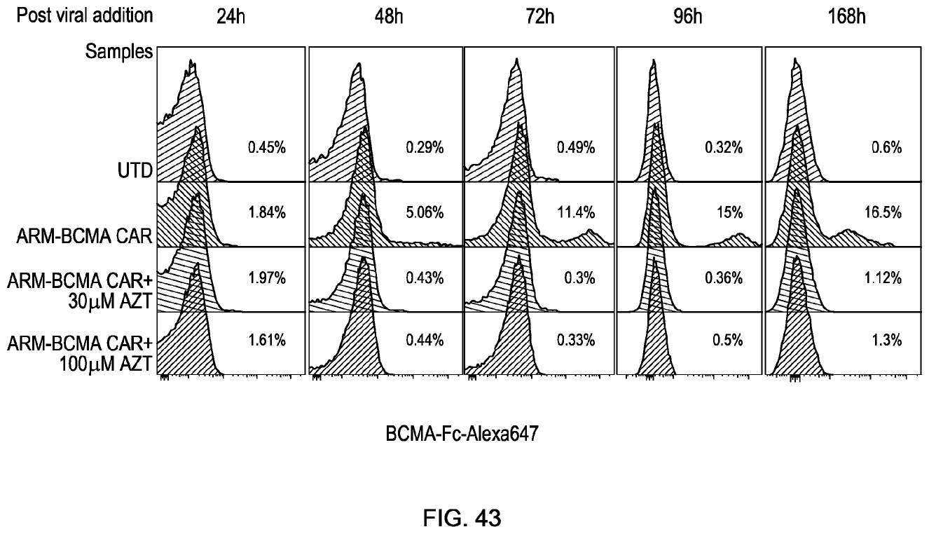

As shown in FIG. 43, FACS analyses indicate that the BCMA-CAR revealed practically no expression at 24 h after the addition of the lentiviral vector. However, the CAR+ population initially emerged at 48 h. The CAR+ population slightly increased at each time point from 48 h to 168 h after viral addition. CAR seemed to be stably expressed starting from 96 h. This contrasts with the untransduced (UTD) and AZT treated samples, which showed no CAR+ population at any time point from 48 h (FIG. 43). AZT was able to effectively inhibit CAR expression at both the 30 μM and 100 μM doses, suggesting that BCMA-CAR expression is due to viral gene integration into the host cell genome and unlikely a consequence of lentiviral pseudo-transduction.

### ARM-BCMA CAR Preserves T Cell Sternness

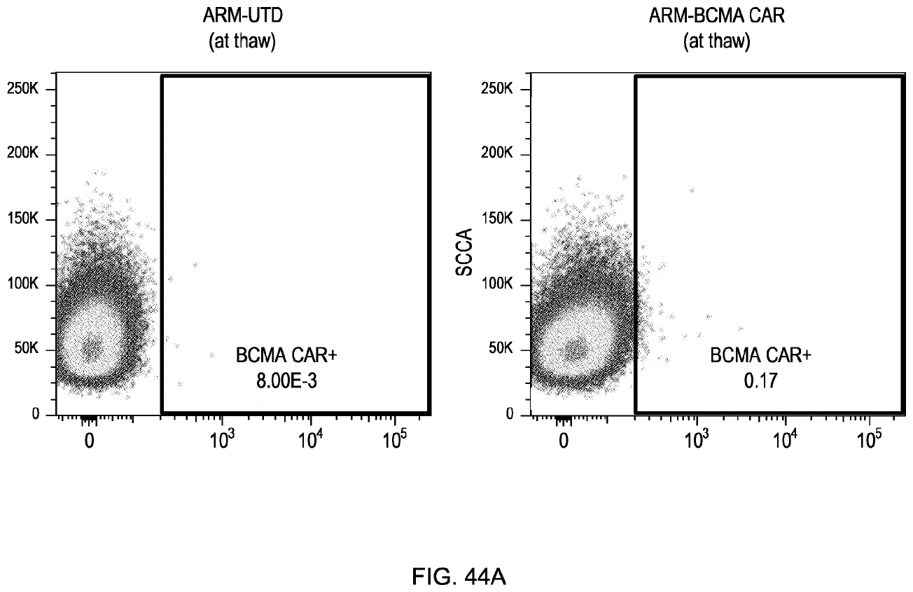

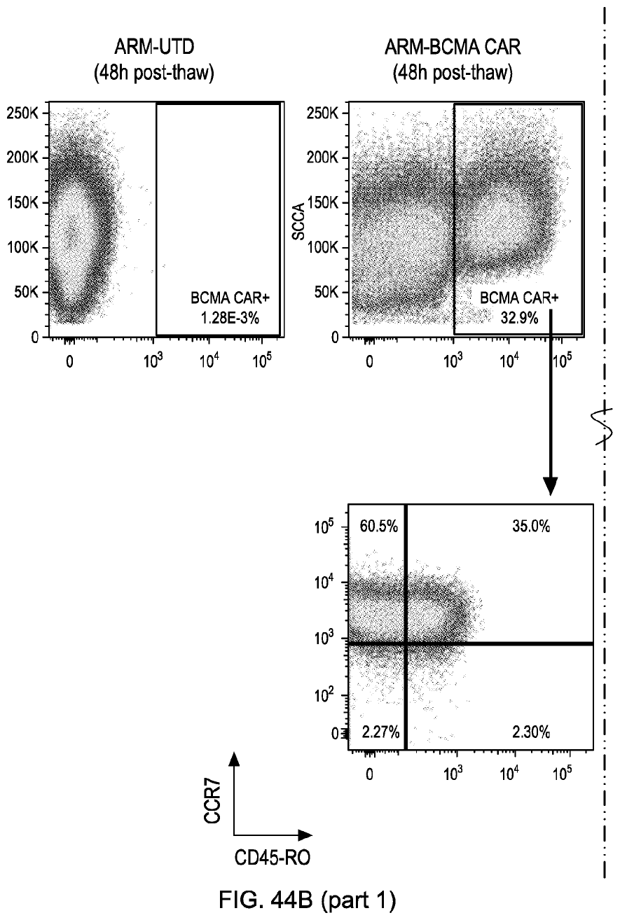

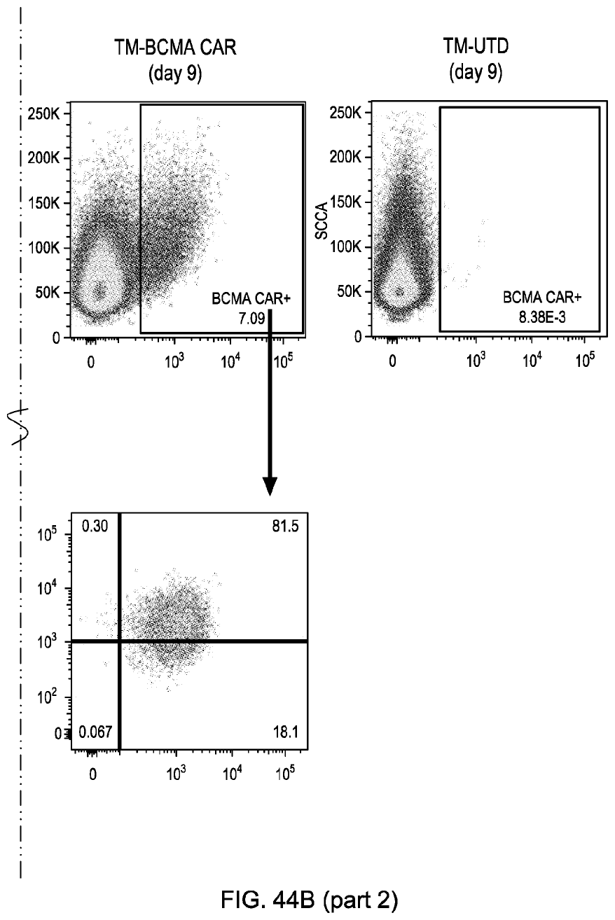

ARM-BCMA CAR and TM-BCMA CAR were analyzed by FACS to evaluate CAR expression at thaw, as well as the T-cell phenotype at 48 h post thaw (FIGS. 44A and 44B). BCMA-CAR was almost undetectable at thaw seen in two donors (FIG. 44A), which is consistent with the observation in the CAR expression kinetics study shown in FIG. 43. However, at 48 h post-thaw, BCMA-CAR expression was 32.9% for ARM-BCMA CAR. In contrast, TM-BCMA CAR revealed BCMA-CAR expression of 7% (FIG. 44B). Analysis of the CAR+ T-cell phenotype revealed that the ARM process retained naïve-like T cells (60% CD45RO−/CCR7+), which proved to be 26 folds more than the effector memory T cell population (CD45RO+/CCR7−). The TM process mainly resulted in central-memory T cells (81% CD45RO+/CCR7+) within CAR+ T cells. The naïve-like T cell population was nearly absent with the TM process. This naïve T-cell population largely overlaps with CD45RO−/CD27+ Tstem cells (described by Cohen A D, et al., (2019) J Clin Invest; 129(6):2210-21; and Fraietta, et al (2018) Nat Med, 24(5); 563-571) and is associated with enhanced CAR-T expansion and clinical responses.

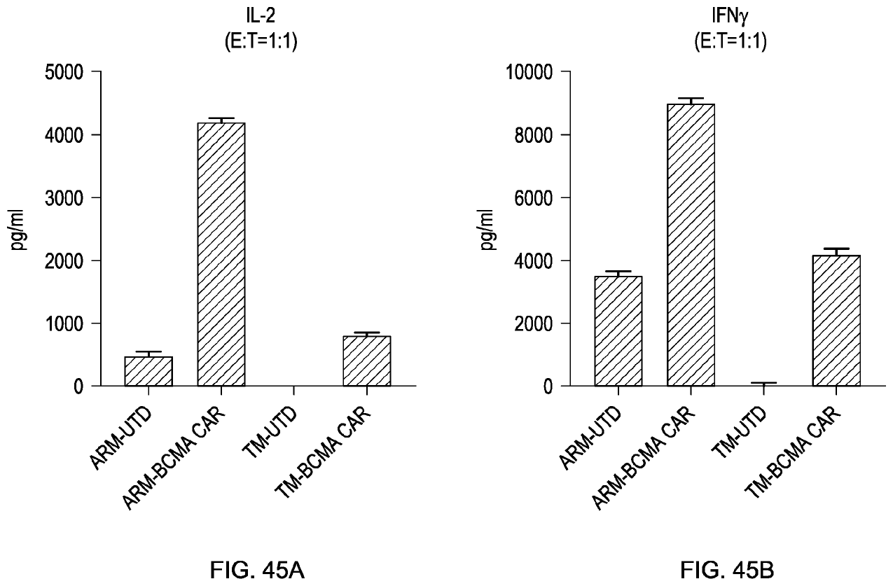

In addition to its phenotype, the final ARM-BCMA CAR cell product was also assessed for its activation in vitro. ARM-BCMA CAR and TM-BCMA CAR were thawed and co-cultured with the BCMA-expressing cell line KMS-11. Post-thaw ARM-BCMA CAR cells were rested for 24 h prior to co-culture being established. Comparing cytokine levels in the supernatants 24 hours after co-culture revealed a 5-fold increase of IL-2 and a 2-fold increase in levels of IFN-γ secreted by ARM-BCMA CAR as compared to TM-BCMA CAR as shown in FIGS. 45A and 45B. Experiments with UTD cells that underwent the ARM or TM process confirmed BCMA-specific recognition by ARM-BCMA CAR and TM-BCMA CAR. However, the higher background of IFN-γ secretion by ARM-UTD in the absence of BCMA-specific stimulation (FIG. 45B) is likely due to the activated nature of the ARM products.

In summary, the ARM process used to generate BCMA CART cells results in T cells with CAR-expression higher than that of the TM process. ARM-BCMA CAR demonstrates BCMA-specific activation in vitro and secretes higher levels of IL-2 as compared to TM-BCMA CAR, which correlates with its Tstem phenotype.

### Efficacy of ARM-BCMA CAR and TM-BCMA CAR in a Xenograft Model

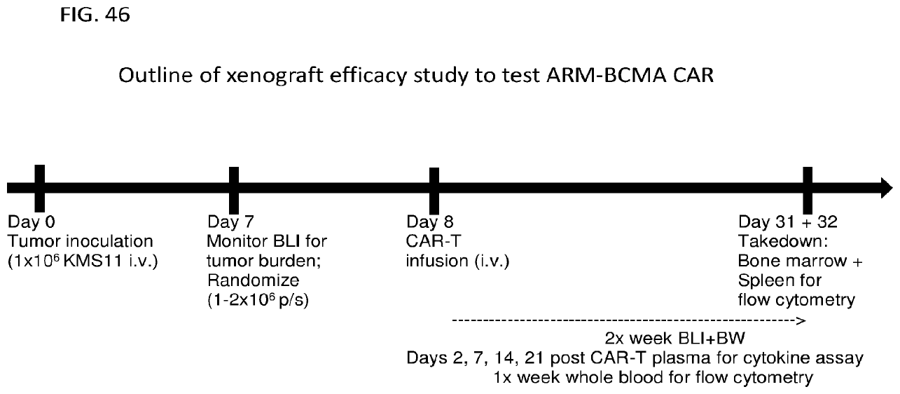

Pharmacology studies in vivo were used to guide the development of ARM-BCMA CAR. For the experiment described in FIG. 46, ARM-BCMA CAR was generated with GMP material. In parallel, TM-BCMA CAR was made using the same batch of T cells but with TM. For dose calculation using ARM-BCMA CAR, the measurement of % CAR+ at 72 h post-thaw of product was used to calculate the dose; while for TM-BCMA CAR, % CAR+ on day 9 TM products was used to calculate the dose. The efficacy of CAR-T cells generated using the different processes was evaluated in immunodeficient NSG mice (NOD-scid IL2Rg-null), which were inoculated with the MM cell line KMS-11-Luc. This tumor cell line engrafts in the bone marrow. Eight days after MM inoculation, cohorts of mice received a single infusion of CAR+ T cells. Doses were normalized to total CAR-T cells for the matched dose group. UTD T cells were prepared similarly and given as an independent group to control for allogeneic response to the tumor. The UTD dose reflected the highest total T cell dose of the respective process we could achieve for both TM and ARM.

TABLE 26
Summary of the study design for different dose
groups, and time points for blood pharmacokinetic
(PK) and plasma cytokine measurement.

| | | | Blood cellular kinetics |
|---|---|---|---|
| Cell | | CAR+ product/ | and plasma cytokine |
| process | Group/arm | mouse | post CAR-T injection |
| | PBS | | Cytokines: Day 2, 7, |
| ARM | UTD | | 14, and 21 |
| | ARM-BCMA CAR | 1.5e⁵ | PK: Day 7, 14, and 21 |
| | ARM-BCMA CAR | 5e⁴ | |
| | ARM-BCMA CAR | 1e⁴ | |
| TM | UTD | | |
| | TM-BCMA CAR | 5e⁵ | |
| | TM-BCMA CAR | 1.5e⁵ | |
| | TM-BCMA CAR | 5e⁴ | |

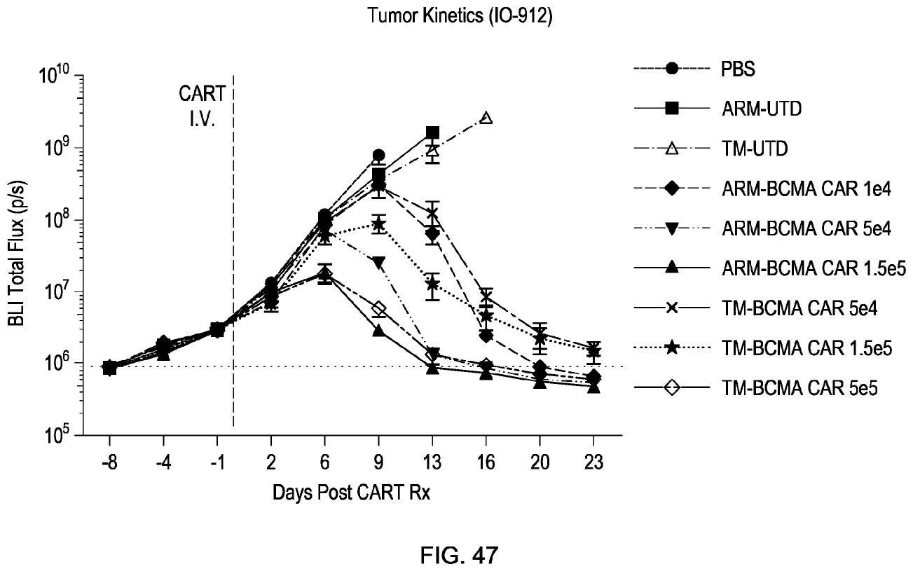

FIG. 47 is the tumor regression curve for all the groups. Both BCMA CAR-T products (ARM-BCMA CAR and TM-BCMA CAR) were able to eliminate tumor at the tested dose levels, even at the lowest dose group. Tumor-regression was induced in a dose-dependent manner. The on-set of effect in tumor-killing was delayed for about a week at the low dose group compared to the high dose group. ARM-BCMA CAR induced similar tumor regression at doses 3-5 folds lower than TM-BCMA CAR, indicating that ARM-BCMA CAR is 3-5 folds more potent compared to TM-BCMA CAR in tumor-killing.

Moreover, in this study, the efficacy of TM-BCMA CAR* was also evaluated. TM-BCMA CAR* and ARM-BCMA CAR expressed the same anti-BCMA CAR, but were manufactured using different processes: the TM process and the ARM process, respectively. The results demonstrated that ARM-BCMA CAR induced similar tumor regression at doses 1-5 folds lower than TM-BCMA CAR*.

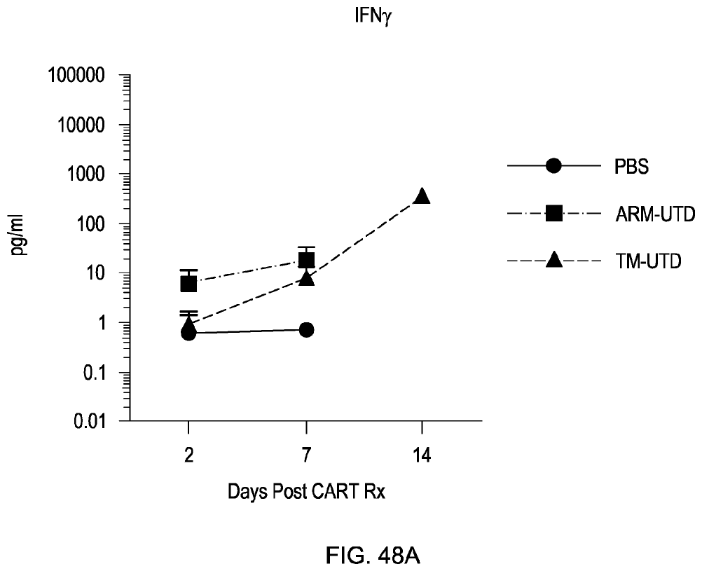

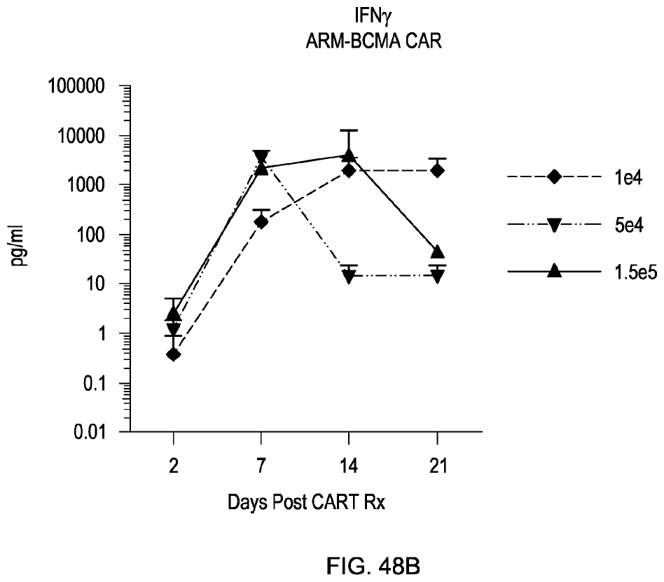

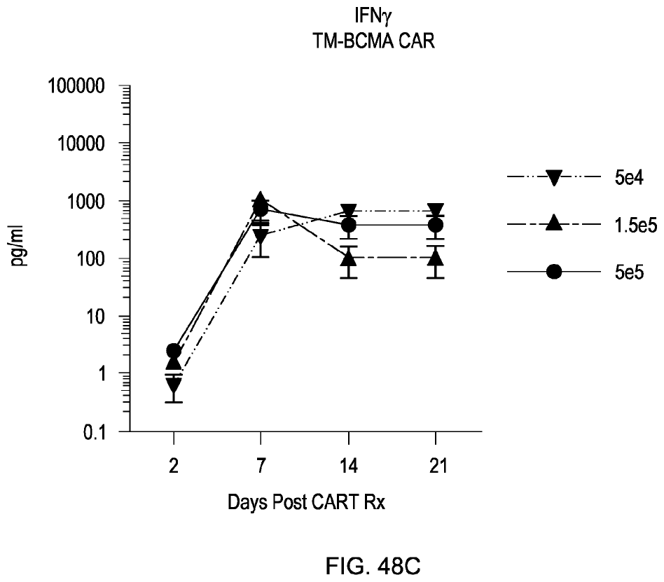

All mice were bled at day 2, 7, 14, and 21 post CAR-T therapy to measure plasma IFN-γ (FIGS. 48A-48C). No early peak was observed and all groups showed very low level of circulating IFN-γ (<10 pg/ml) at day 2. Peaks for all the groups were observed within 14 days post CAR-T dose. However, IFN-γ levels were 3.5-fold higher for ARM-BCMA CAR compared to TM-BCMA CAR. ARM-UTD groups produce little or no IFN-γ at day 2 and day 7 prior to study termination. IFN-γ declined in the higher dose groups at day 21, when compared to the ARM-BCMA CAR 1e4 and TM-BCMA CAR 5e4 groups as the CAR+T cells were still expanding with delayed tumor inhibition in these two groups.

### In Vivo ARM-BCMA CAR Cellular Kinetics

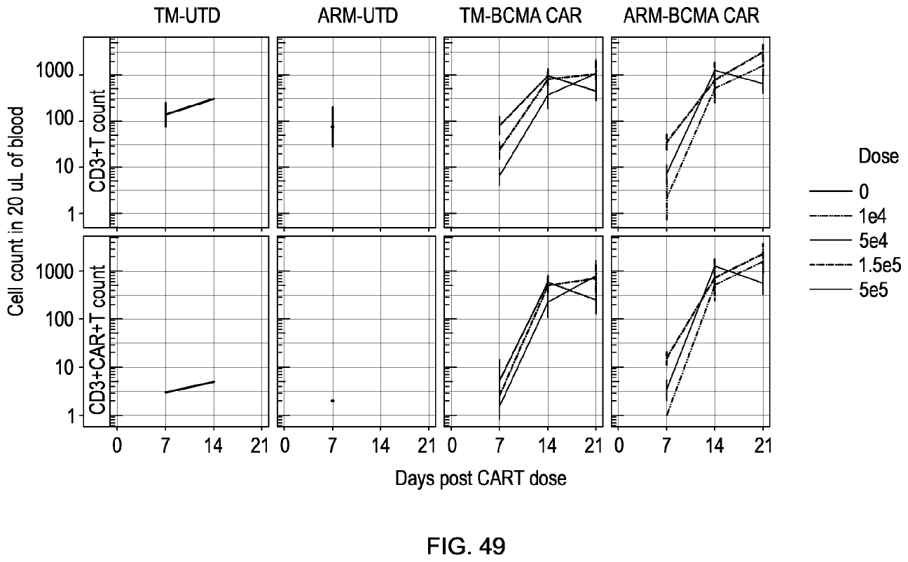

As part of this pharmacology study to assess efficacy in NSG mice, the expansion of peripheral blood CAR-T cells was analyzed by FACS up to 3 weeks after infusion. Both CD3+ T cell and CAR+ T cell expansion were observed in all CAR-T treated groups. There was no clear dose-dependent expansion for ARM-BCMA CAR or TM-BCMA CAR with respect to Cmax or AUCO-21d. The peak of cellular expansion for ARM-BCMA CAR or MTV273 was not achieved within 21 days. However, TM-BCMA CAR at dose group of 5e5 and ARM-BCMA CAR at dose group of 0.5e5 achieved apparent peak expansion at day 14 (FIG. 49). Comparing the expansion of ARM-BCMA CAR with that of TM-BCMA CAR in 21 days, both Cmax and AUCO-21d of ARM-BCMA CAR were 2 to 3 times higher.

### Example 14

### Manufacturing BCMA CART Cells Using the Activated Rapid Manufacturing (ARM) Process Using IL-15 or hetIL-15 (IL-15/sIL-15Ra)

The ARM process of BCMA CART cells initiates with the preparation of the media as outlined in Table 25.

Cryopreserved leukapheresis product is used as the starting material and is processed for T cell enrichment. When available, the apheresis paper work is utilized to define the T cell percentage. In the absence of the T cell percentage data on the apheresis paperwork, the sentinel vial testing is performed on incoming cryopreserved leukapheresis products to obtain T cell percentage target for the apheresis. The results for the T cell percentage determine how many bags are thawed on Day 0 of the ARM process.

Cryopreserved leukapheresis is thawed, washed, and then undergoes T cell selection and enrichment using CliniMACS® microbead technology. Viable nucleated cells (VNCs) are activated with TransACT (Miltenyi) and transduced with a lentiviral vector encoding the CAR. The viable cells selected with the Miltenyi microbeads are seeded into the centricult on the Prodigy®, which is a non-humidified incubation chamber. While in culture, the cells are suspended in Rapid media, which is an OpTmizer™ CTS™ based medium that contains the CTS™ Supplement (ThermoFisher), Glutamax, IL-15 or hetIL-15 (IL-15/sIL-15Ra), and 2% Immune cell serum replacement amongst its components to promote T cell activation and transduction. Lentiviral transduction is performed once on the day of seeding after the TransACT has been added to the diluted cells in the culture media. Lentiviral vector will be thawed immediately prior to use on day of seeding for up to 30 minutes at room temperature.

From the start of the process on Day 0 to the initiation of the culture wash and harvest, BCMA CART cells are cultured for 20-28 hours from seeding. Following culture, the cell suspension undergoes two culture washes and one harvest wash within the centricult chamber (Miltenyi Biotech).

After the harvest wash on the CliniMACS® Prodigy® on day 1, the cell suspension is sampled to determine viable cell count and viability. Cell suspension is then transferred to a centrifuge to be pelleted manually. The supernatant is removed, and the cell pellet is re-suspended in CS10 (BioLife Solution), resulting in a product formulation with a final DMSO concentration of ˜10.0%. The viable cell count is formulated at the end of harvest for dosing. The doses are then distributed into individual cryobags and analytical sampling into cryovials.

Cryopreserved products are stored in monitored LN2 storage tanks, in a secure, limited access area until final release and shipping.

In some embodiments, IL-15 or hetIL-15 used in the OpTmizer™ CTS™ based medium can be replaced with IL-6 or IL-6/sIL-6Ra.

### Example 15

### Manufacturing CD19 CART Cells Using the Activated Rapid Manufacturing (ARM) Process

The ARM process of CD19 CART cells initiates with the preparation of the media as outlined in Table 25.

Cryopreserved leukapheresis product is used as the starting material and is processed for T cell enrichment. When available, the apheresis paper work is utilized to define the T cell percentage. In the absence of the T cell percentage data on the apheresis paperwork, the sentinel vial testing is performed on incoming cryopreserved leukapheresis products to obtain T cell percentage target for the apheresis. The results for the T cell percentage determine how many bags are thawed on Day 0 of the ARM process.

Cryopreserved leukapheresis is thawed, washed, and then undergoes T cell selection and enrichment using CliniMACS® microbead technology. Viable nucleated cells (VNCs) are activated with TransACT (Miltenyi) and transduced with a lentiviral vector encoding the CAR. The viable cells selected with the Miltenyi microbeads are seeded into the centricult on the Prodigy®, which is a non-humidified incubation chamber. While in culture, the cells are suspended in Rapid media, which is an OpTmizer™ CTS™ based medium that contains the CTS™ Supplement (ThermoFisher), Glutamax, IL-2 and 2% Immune cell serum replacement amongst its components to promote T cell activation and transduction. Lentiviral transduction is performed once on the day of seeding after the TransACT has been added to the diluted cells in the culture media. Lentiviral vector will be thawed immediately prior to use on day of seeding for up to 30 minutes at room temperature.

From the start of the process on Day 0 to the initiation of the culture wash and harvest, CD19 CART cells are cultured for 20-28 hours from seeding. Following culture, the cell suspension undergoes two culture washes and one harvest wash within the centricult chamber (Miltenyi Biotech).

After the harvest wash on the CliniMACS® Prodigy® on day 1, the cell suspension is sampled to determine viable cell count and viability. Cell suspension is then transferred to a centrifuge to be pelleted manually. The supernatant is removed, and the cell pellet is re-suspended in CS10 (BioLife Solution), resulting in a product formulation with a final DMSO concentration of ˜10.0%. The viable cell count is formulated at the end of harvest for dosing. The doses are then distributed into individual cryobags and analytical sampling into cryovials.

Cryopreserved products are stored in monitored LN2 storage tanks, in a secure, limited access area until final release and shipping.

In some embodiments, IL-2 used in the OpTmizer™ CTS™ based medium can be replaced with IL-15, hetIL-15 (IL-15/sIL-15Ra), IL-6, or IL-6/sIL-6Ra.

### Example 16

### Manufacturing CAR19.HilD-Expressing T Cells Using the Activated Rapid Manufacturing (ARM) Process

This example describes manufacturing CAR19.HilD-expressing T cells using the ARM process. CAR19 is an anti-CD19 CAR. CAR19.HilD refers to a construct where CAR19 is fused to a HilD-tag. The HilD-tag (also referred to as “IKZF3 136-180 and 236-249”) is an IKZF3-based degradation tag that can mediate lenalidomide-dependent degradation of a target protein. The HilD-tag includes amino acid residues 136-180 and 236-249 of human IKZF3 and comprises the amino acid sequence of SEQ ID NO: 312.

### Methods

Human primary T cells from two donors were thawed and prepared with TransAct in a conical tube. CAR19 and CAR19.HilD vectors were added at two different MOIs (Multiplicity of Infection): 1 and 2. Untransduced T cells were also cultured to serve as a control. Cells were cultured with or without Lenalidomide at 1 μM.

After a 24-hr incubation, cells were washed using PBS containing 1% HSA (human serum albumin) and then re-suspended in pre-warmed Optimizer media. Harvested cells were counted and viability was measured using Cellaca's system. To determine the transduction efficiency, cells were re-plated into a fresh 24-well plate and incubated at 37° C., in a humidifier chamber, under a 5% CO₂ atmosphere.

At the end of day 6 post vector wash, all the cells were harvested and washed with PBS. The cells are stained for live dead stain followed by surface staining using an anti-CD3 antibody (staining for T cells) and an anti-ID antibody (staining for CAR expression). The cells were then washed twice with PBS and re-suspended in 2% Paraformaldehyde fixation buffer, for 10 mins, at room temperature. The fixed cells were washed with PBS and then acquired in a Fortessa instrument. The results were analyzed using Flow Jo software.

For analysis, the cells were pre-gated on singlet, viable and CD3 positive cells which were then gated for CAR expression, using untransduced cells as the control.

### Result

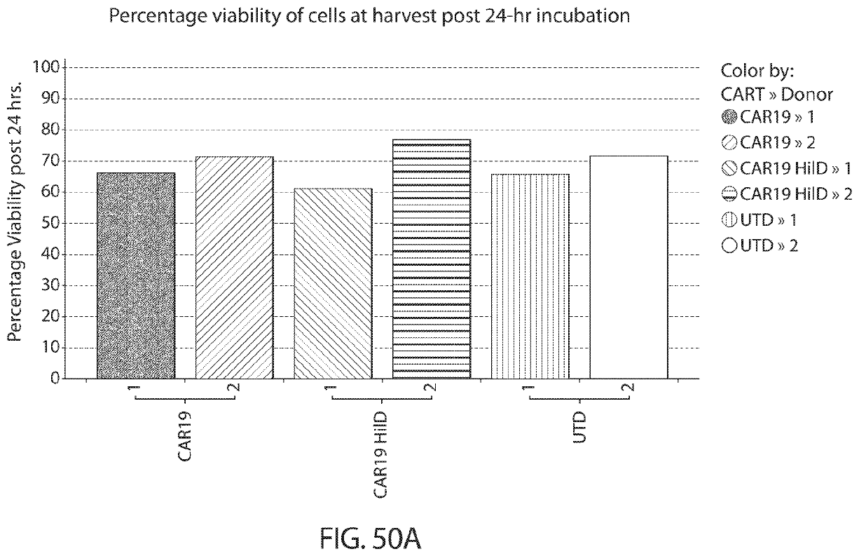

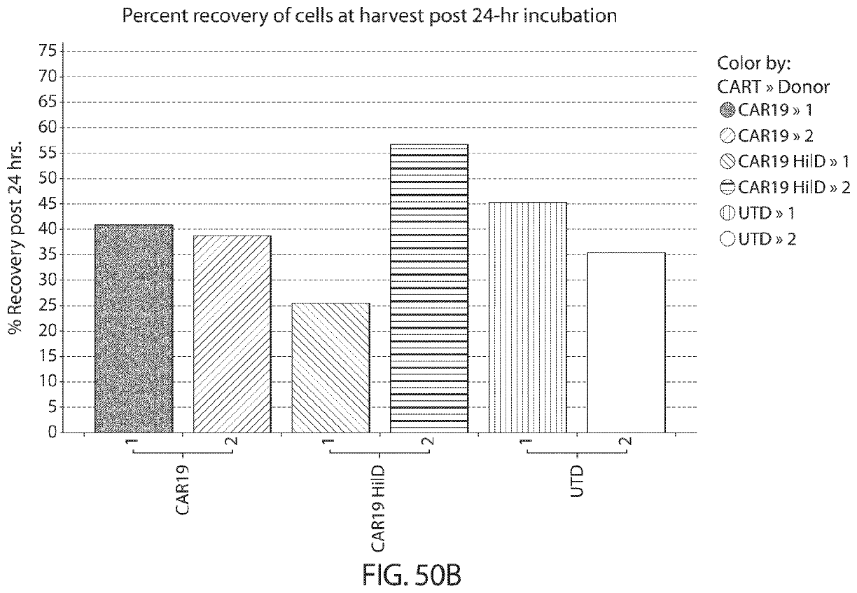

CAR19.HilD cells showed similar viability as CAR19 cells and untransduced cells (UTD), with a percent viability around 40-50% post 24-hr incubation with the viral vector and the TransAct (FIGS. 50A). Recovery of CART19.HilD cells, manufactured using the ARM process, was around 50%, at 24 hrs (FIG. 50B). A similar result was obtained with untagged CART19 cells (FIG. 50B). Thus, appending the HilD tag to CAR19 does not impact viability or recovery of the cells manufactured using the ARM process.

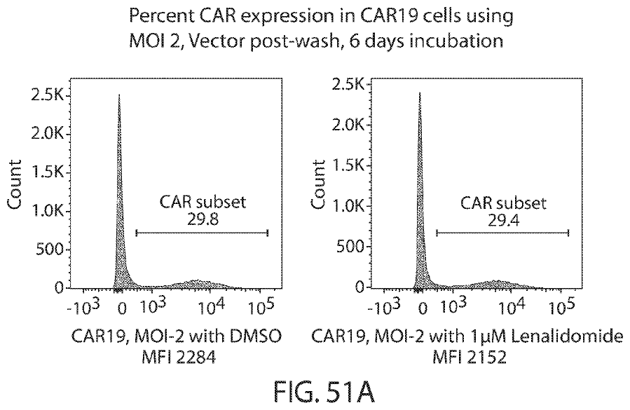

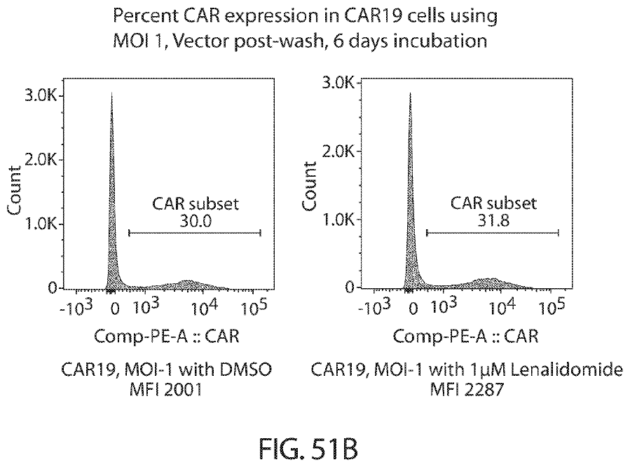

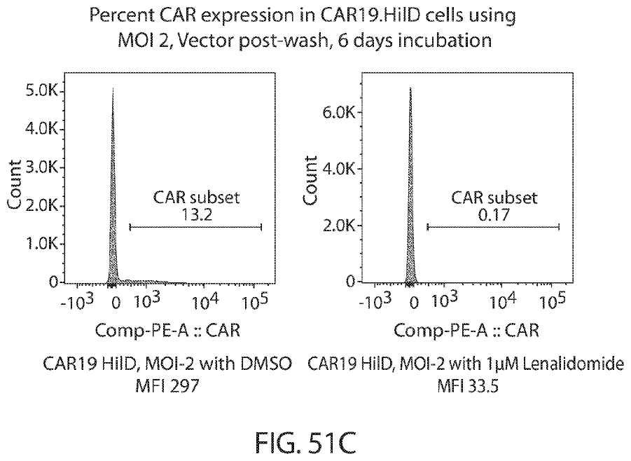

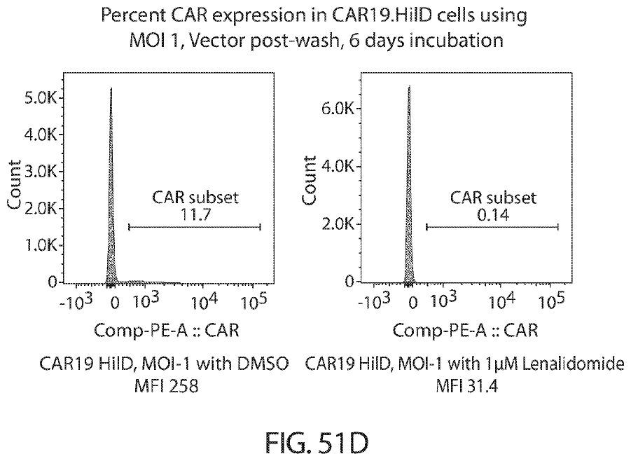

In terms of CAR expression, CART19.HilD cells showed 14% CAR expression, 6 days post-wash of the vector. This CAR expression dropped below 1% in the presence of lenalidomide, showing the effect of the drug on regulating CAR19-HilD expression (FIGS. 51C and 51D). This effect of lenalidomide was specific to CAR19-HilD, as addition of lenalidomide to untagged CART19 cells had no influence on CAR expression (transduction efficiency obtained around 29-30%) (FIGS. 51A and 51B).

### EQUIVALENTS

The disclosures of each and every patent, patent application, and publication cited herein are hereby incorporated herein by reference in their entirety. While this invention has been disclosed with reference to certain embodiments, it is apparent that further embodiments and variations of this invention may be devised by others skilled in the art without departing from the true spirit and scope of the invention. The appended claims are intended to be construed to include all such embodiments and equivalent variations.

What is claimed is:

1. A method of making a population of cells (for example, T cells) that comprise:
a first nucleic acid molecule that encodes a controllable chimeric antigen receptor (CCAR), or
a second nucleic acid molecule that encodes a chimeric antigen receptor (CAR) and a regulatory molecule,
the method comprising:
(i) contacting (for example, binding) a population of cells (for example, T cells, for example, T cells isolated from a frozen or fresh leukapheresis product) with an agent that stimulates a CD3/TCR complex and/or an agent that stimulates a costimulatory molecule on the surface of the cells;
(ii) contacting the population of cells (for example, T cells) with a first nucleic acid molecule (for example, a DNA or RNA molecule) encoding a CCAR or a second nucleic acid molecule (for example, a DNA or RNA molecule) encoding a CAR and a regulatory molecule, thereby providing a population of cells (for example, T cells) comprising the first or second nucleic acid molecule, and
(iii) harvesting the population of cells (for example, T cells) for storage (for example, reformulating the population of cells in cryopreservation media) or administration, wherein:
(a) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and
step (iii) is performed no later than 30 (for example, 26) hours after the beginning of step (i), for example, no later than 22, 23, 24, 25, 26, 27, 28, 29, or 30 hours after the beginning of step (i), for example, no later than 24 hours after the beginning of step (i),
(b) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and
step (iii) is performed no later than 30 hours after the beginning of step (ii), for example, no later than 22, 23, 24, 25, 26, 27, 28, 29, or 30 hours after the beginning of step (ii), or
(c) the population of cells from step (iii) are not expanded, or expanded by no more than 5, 10, 15, 20, 25, 30, 35, or 40%, for example, no more than 10%, for example, as assessed by the number of living cells, compared to the population of cells at the beginning of step (i),
optionally wherein the first or second nucleic acid molecule in step (ii) is on a viral vector, optionally wherein the first or second nucleic acid molecule in step (ii) is an RNA molecule on a viral vector, optionally wherein step (ii) comprises transducing the population of cells (for example, T cells) with a viral vector comprising the first or second nucleic acid molecule.

2. The method of claim 1, wherein the agent that stimulates a CD3/TCR complex is an agent that stimulates CD3 (for example, an anti-CD3 antibody) and wherein the agent that stimulates a costimulatory molecule is an agent that stimulates CD28, ICOS, CD27, HVEM, LIGHT, CD40, 4-1BB, OX40, DR3, GITR, CD30, TIM1, CD2, CD226, or any combination thereof, optionally wherein the agent that stimulates a CD3/TCR complex or the agent that stimulates a costimulatory molecule is chosen from an antibody (for example, a single-domain antibody (for example, a heavy chain variable domain antibody), a peptibody, a Fab fragment, or a scFv), a small molecule, or a ligand (for example, a naturally-existing, recombinant, or chimeric ligand), optionally wherein the agent that stimulates a CD3/TCR complex or the agent that stimulates a costimulatory molecule does not comprise a bead, optionally wherein the agent that stimulates a CD3/TCR complex comprises an anti-CD3 antibody and the agent that stimulates a costimulatory molecule comprises an anti-CD28 antibody, optionally wherein the agent that stimulates a CD3/TCR complex comprises an anti-CD3 antibody covalently attached to a colloidal polymeric nanomatrix and the agent that stimulates a costimulatory molecule comprises an anti-CD28 antibody covalently attached to a colloidal polymeric nanomatrix, optionally wherein the agent that stimulates a CD3/TCR complex and the agent that stimulates a costimulatory molecule comprise T Cell TransAct™.

3. The method of claim 1 or 2, wherein step (i) increases the percentage of cells that comprise the first or second nucleic acid molecule in the population of cells from step (iii), for example, the population of cells from step (iii) shows a higher percentage of cells that comprise the first or second nucleic acid molecule (for example, at least 10, 20, 30, 40, 50, or 60% higher), compared with cells made by an otherwise similar method without step (i).

4. The method of any one of claims 1-3, wherein:
(a) the percentage of naïve cells, for example, naïve T cells, for example, CD45RA+ CD45RO− CCR7+ T cells, in the population of cells from step (iii) is the same as or differs by no more than 5 or 10% from the percentage of naïve cells, for example, naïve T cells, for example, CD45RA+ CD45RO− CCR7+ cells, in the population of cells at the beginning of step (i);
(b) the percentage of naïve cells, for example, naïve T cells, for example, CD45RA+ CD45RO− CCR7+ T cells, in the population of cells from step (iii) is increased by, for example, at least 1.2, 1.4, 1.6, 1.8, 2.0, 2.2, 2.4, 2.6, 2.8, or 3-fold, as compared to the percentage of naïve cells, for example, naïve T cells, for example, CD45RA+ CD45RO− CCR7+ cells, in the population of cells at the beginning of step (i);
(c) the percentage of naïve T cells that comprise the first or second nucleic acid molecule, for example, CD45RA+ CD45RO− CCR7+ T cells that comprise the first or second nucleic acid molecule, in the population of cells increases during the duration of step (ii), for example, increases by, for example, at least 30, 35, 40, 45, 50, 55, or 60%, between 18-24 hours after the beginning of step (ii); or
(d) the percentage of naïve cells, for example, naïve T cells, for example, CD45RA+ CD45RO− CCR7+ T cells, in the population of cells from step (iii) does not decrease, or decreases by no more than 5 or 10%, as compared to the percentage of naïve cells, for example, naïve T cells, for example, CD45RA+ CD45RO− CCR7+ cells, in the population of cells at the beginning of step (i).

5. The method of any one of claims 1-4, wherein:
(a) the population of cells from step (iii) shows a higher percentage of naïve cells, for example, naïve T cells, for example, CD45RA+ CD45RO− CCR7+ T cells (for example, at least 10, 20, 30, or 40% higher), compared with cells made by an otherwise similar method in which step (iii) is performed more than 26 hours after the beginning of step (i), for example, more than 5, 6, 7, 8, 9, 10, 11, or 12 days after the beginning of step (i);
(b) the percentage of naïve cells, for example, naïve T cells, for example, CD45RA+ CD45RO− CCR7+ T cells, in the population of cells from step (iii) is higher (for example, at least 1.2, 1.4, 1.6, 1.8, 2.0, 2.2, 2.4, 2.6, 2.8, or 3-fold higher) than the percentage of naïve cells, for example, naïve T cells, for example, CD45RA+ CD45RO− CCR7+ T cells, in cells made by an otherwise similar method in which step (iii) is performed more than 26 hours after the beginning of step (i), for example, more than 5, 6, 7, 8, 9, 10, 11, or 12 days after the beginning of step (i);
(c) the percentage of naïve T cells that comprise the first or second nucleic acid molecule, for example, CD45RA+ CD45RO− CCR7+ T cells that comprise the first or second nucleic acid molecule, in the population of cells from step (iii) is higher (for example, at least 4, 6, 8, 10, or 12-fold higher) than the percentage of naïve T cells that comprise the first or second nucleic acid molecule, for example, CD45RA+ CD45RO− CCR7+ T cells that comprise the first or second nucleic acid molecule, in cells made by an otherwise similar method in which step (iii) is performed more than 26 hours after the beginning of step (i), for example, more than 5, 6, 7, 8, 9, 10, 11, or 12 days after the beginning of step (i);
(d) the population of cells from step (iii) shows a higher percentage of naïve cells, for example, naïve T cells, for example, CD45RA+ CD45RO− CCR7+ T cells (for example, at least 10, 20, 30, or 40% higher), compared with cells made by an otherwise similar method which further comprises, after step (ii) and prior to step (iii), expanding the population of cells (for example, T cells) in vitro for more than 3 days, for example, for 5, 6, 7, 8 or 9 days;
(e) the percentage of naïve cells, for example, naïve T cells, for example, CD45RA+ CD45RO− CCR7+ T cells, in the population of cells from step (iii) is higher (for example, at least 1.2, 1.4, 1.6, 1.8, 2.0, 2.2, 2.4, 2.6, 2.8, or 3-fold higher) than the percentage of naïve cells, for example, naïve T cells, for example, CD45RA+ CD45RO− CCR7+ T cells, in cells made by an otherwise similar method which further comprises, after step (ii) and prior to step (iii), expanding the population of cells (for example, T cells) in vitro for more than 3 days, for example, for 5, 6, 7, 8 or 9 days; or
(f) the percentage of naïve T cells that comprise the first or second nucleic acid molecule, for example, CD45RA+ CD45RO− CCR7+ T cells that comprise the first or second nucleic acid molecule, in the population of cells from step (iii) is higher (for example, at least 4, 6, 8, 10, or 12-fold higher) than the percentage of naïve T cells that comprise the first or second nucleic acid molecule, for example, CD45RA+ CD45RO− CCR7+ T cells that comprise the first or second nucleic acid molecule, in cells made by an otherwise similar method which further comprises, after step (ii) and prior to step (iii), expanding the population of cells (for example, T cells) in vitro for more than 3 days, for example, for 5, 6, 7, 8 or 9 days.

6. The method of any one of claims 1-5, wherein:
(a) the percentage of central memory cells, for example, central memory T cells, for example, CD95+ central memory T cells, in the population of cells from step (iii) is the same as or differs by no more than 5 or 10% from the percentage of central memory cells, for example, central memory T cells, for example, CD95+ central memory T cells, in the population of cells at the beginning of step (i);
(b) the percentage of central memory cells, for example, central memory T cells, for example, CCR7+CD45RO+ T cells, in the population of cells from step (iii) is reduced by at least 20, 25, 30, 35, 40, 45, or 50%, as compared to the percentage of central memory cells, for example, central memory T cells, for example, CCR7+CD45RO+ T cells, in the population of cells at the beginning of step (i);
(c) the percentage of central memory T cells that comprise the first or second nucleic acid molecule, for example, CCR7+CD45RO+ cells that comprise the first or second nucleic acid molecule, decreases during the duration of step (ii), for example, decreases by, for example, at least 8, 10, 12, 14, 16, 18, or 20%, between 18-24 hours after the beginning of step (ii); or
(d) the percentage of central memory cells, for example, central memory T cells, for example, CCR7+CD45RO+ T cells, in the population of cells from step (iii) does not increase, or increases by no more than 5 or 10%, as compared to the percentage of central memory cells, for example, central memory T cells, for example, CCR7+CD45RO+ T cells, in the population of cells at the beginning of step (i).

7. The method of any one of claims 1-6, wherein:
(a) the population of cells from step (iii) shows a lower percentage of central memory cells, for example, central memory T cells, for example, CD95+ central memory T cells (for example, at least 10, 20, 30, or 40% lower), compared with cells made by an otherwise similar method in which step (iii) is performed more than 26 hours after the beginning of step (i), for example, more than 5, 6, 7, 8, 9, 10, 11, or 12 days after the beginning of step (i);
(b) the percentage of central memory cells, for example, central memory T cells, for example, CCR7+CD45RO+ T cells in the population of cells from step (iii) is lower (for example, at least 20, 30, 40, or 50% lower) than the percentage of central memory cells, for example, central memory T cells, for example, CCR7+CD45RO+ T cells, in cells made by an otherwise similar method in which step (iii) is performed more than 26 hours after the beginning of step (i), for example, more than 5, 6, 7, 8, 9, 10, 11, or 12 days after the beginning of step (i);
(c) the percentage of central memory T cells that comprise the first or second nucleic acid molecule, for example, CCR7+CD45RO+ T cells that comprise the first or second nucleic acid molecule, in the population of cells from step (iii) is lower (for example, at least 10, 20, 30, or 40% lower) than the percentage of central memory T cells that comprise the first or second nucleic acid molecule, for example, CCR7+CD45RO+ T cells that comprise the first or second nucleic acid molecule, in cells made by an otherwise similar method in which step (iii) is performed more than 26 hours after the beginning of step (i), for example, more than 5, 6, 7, 8, 9, 10, 11, or 12 days after the beginning of step (i);
(d) the population of cells from step (iii) shows a lower percentage of central memory cells, for example, central memory T cells, for example, CD95+ central memory T cells (for example, at least 10, 20, 30, or 40% lower), compared with cells made by an otherwise similar method which further comprises, after step (ii) and prior to step (iii), expanding the population of cells (for example, T cells) in vitro for more than 3 days, for example, for 5, 6, 7, 8 or 9 days;
(e) the percentage of central memory cells, for example, central memory T cells, for example, CCR7+CD45RO+ T cells in the population of cells from step (iii) is lower (for example, at least 20, 30, 40, or 50% lower) than the percentage of central memory cells, for example, central memory T cells, for example, CCR7+CD45RO+ T cells, in cells made by an otherwise similar method which further comprises, after step (ii) and prior to step (iii), expanding the population of cells (for example, T cells) in vitro for more than 3 days, for example, for 5, 6, 7, 8 or 9 days; or
(f) the percentage of central memory T cells that comprise the first or second nucleic acid molecule, for example, CCR7+CD45RO+ T cells that comprise the first or second nucleic acid molecule, in the population of cells from step (iii) is lower (for example, at least 10, 20, 30, or 40% lower) than the percentage of central memory T cells that comprise the first or second nucleic acid molecule, for example, CCR7+CD45RO+ T cells that comprise the first or second nucleic acid molecule, in cells made by an otherwise similar method which further comprises, after step (ii) and prior to step (iii), expanding the population of cells (for example, T cells) in vitro for more than 3 days, for example, for 5, 6, 7, 8 or 9 days.

8. The method of any one of claims 1-7, wherein:
(a) the percentage of stem memory T cells, for example, CD45RA+CD95+IL-2 receptor β+CCR7+CD62L+ T cells, in the population of cells from step (iii) is increased, as compared to the percentage of stem memory T cells, for example, CD45RA+CD95+IL-2 receptor β+CCR7+CD62L+ T cells, in the population of cells at the beginning of step (i);
(b) the percentage of stem memory T cells that comprise the first or second nucleic acid molecule, for example, CD45RA+CD95+IL-2 receptor β+CCR7+CD62L+ T cells that comprise the first or second nucleic acid molecule, in the population of cells from step (iii) is increased, as compared to the percentage of stem memory T cells that comprise the first or second nucleic acid molecule, for example, CD45RA+CD95+IL-2 receptor β+CCR7+CD62L+ T cells that comprise the first or second nucleic acid molecule, in the population of cells at the beginning of step (i);
(c) the percentage of stem memory T cells, for example, CD45RA+CD95+IL-2 receptor β+CCR7+CD62L+ T cells, in the population of cells from step (iii) is higher than the percentage of stem memory T cells, for example, CD45RA+CD95+IL-2 receptor β+CCR7+CD62L+ T cells, in cells made by an otherwise similar method in which step (iii) is performed more than 26 hours after the beginning of step (i), for example, more than 5, 6, 7, 8, 9, 10, 11, or 12 days after the beginning of step (i); or
(d) the percentage of stem memory T cells that comprise the first or second nucleic acid molecule, for example, CD45RA+CD95+IL-2 receptor β+CCR7+CD62L+ T cells that comprise the first or second nucleic acid molecule, in the population of cells from step (iii) is higher than the percentage of stem memory T cells that comprise the first or second nucleic acid molecule, for example, CD45RA+CD95+IL-2 receptor β+CCR7+CD62L+ T cells that comprise the first or second nucleic acid molecule, in cells made by an otherwise similar method in which step (iii) is performed more than 26 hours after the beginning of step (i), for example, more than 5, 6, 7, 8, 9, 10, 11, or 12 days after the beginning of step (i);
(e) the percentage of stem memory T cells, for example, CD45RA+CD95+IL-2 receptor β+CCR7+CD62L+ T cells, in the population of cells from step (iii) is higher than the percentage of stem memory T cells, for example, CD45RA+CD95+IL-2 receptor β+CCR7+CD62L+ T cells, in cells made by an otherwise similar method which further comprises, after step (ii) and prior to step (iii), expanding the population of cells (for example, T cells) in vitro for more than 3 days, for example, for 5, 6, 7, 8 or 9 days; or
(f) the percentage of stem memory T cells that comprise the first or second nucleic acid molecule, for example, CD45RA+CD95+IL-2 receptor β+CCR7+CD62L+ T cells that comprise the first or second nucleic acid molecule, in the population of cells from step (iii) is higher than the percentage of stem memory T cells that comprise the first or second nucleic acid molecule, for example, CD45RA+CD95+IL-2 receptor β+CCR7+CD62L+ T cells that comprise the first or second nucleic acid molecule, in cells made by an otherwise similar method which further comprises, after step (ii) and prior to step (iii), expanding the population of cells (for example, T cells) in vitro for more than 3 days, for example, for 5, 6, 7, 8 or 9 days.

9. The method of any one of claims 1-8, wherein:
(a) the median GeneSetScore (Up TEM vs. Down TSCM) of the population of cells from step (iii) is about the same as or differs by no more than (for example, increased by no more than) about 25, 50, 75, 100, or 125% from the median GeneSetScore (Up TEM vs. Down TSCM) of the population of cells at the beginning of step (i);
(b) the median GeneSetScore (Up TEM vs. Down TSCM) of the population of cells from step (iii) is lower (for example, at least about 100, 150, 200, 250, or 300% lower) than the median GeneSetScore (Up TEM vs. Down TSCM) of:
cells made by an otherwise similar method in which step (iii) is performed more than 26 hours after the beginning of step (i), for example, more than 5, 6, 7, 8, 9, 10, 11, or 12 days after the beginning of step (i), or
cells made by an otherwise similar method which further comprises, after step (ii) and prior to step (iii), expanding the population of cells (for example, T cells) in vitro for more than 3 days, for example, for 5, 6, 7, 8 or 9 days;
(c) the median GeneSetScore (Up Treg vs. Down Teff) of the population of cells from step (iii) is about the same as or differs by no more than (for example, increased by no more than) about 25, 50, 100, 150, or 200% from the median GeneSetScore (Up Treg vs. Down Teff) of the population of cells at the beginning of step (i);
(d) the median GeneSetScore (Up Treg vs. Down Teff) of the population of cells from step (iii) is lower (for example, at least about 50, 100, 125, 150, or 175% lower) than the median GeneSetScore (Up Treg vs. Down Teff) of:
cells made by an otherwise similar method in which step (iii) is performed more than 26 hours after the beginning of step (i), for example, more than 5, 6, 7, 8, 9, 10, 11, or 12 days after the beginning of step (i), or
cells made by an otherwise similar method which further comprises, after step (ii) and prior to step (iii), expanding the population of cells (for example, T cells) in vitro for more than 3 days, for example, for 5, 6, 7, 8 or 9 days;
(e) the median GeneSetScore (Down stemness) of the population of cells from step (iii) is about the same as or differs by no more than (for example, increased by no more than) about 25, 50, 100, 150, 200, or 250% from the median GeneSetScore (Down stemness) of the population of cells at the beginning of step (i);
(f) the median GeneSetScore (Down stemness) of the population of cells from step (iii) is lower (for example, at least about 50, 100, or 125% lower) than the median GeneSetScore (Down stemness) of:
cells made by an otherwise similar method in which step (iii) is performed more than 26 hours after the beginning of step (i), for example, more than 5, 6, 7, 8, 9, 10, 11, or 12 days after the beginning of step (i), or
cells made by an otherwise similar method which further comprises, after step (ii) and prior to step (iii), expanding the population of cells (for example, T cells) in vitro for more than 3 days, for example, for 5, 6, 7, 8 or 9 days;
(g) the median GeneSetScore (Up hypoxia) of the population of cells from step (iii) is about the same as or differs by no more than (for example, increased by no more than) about 125, 150, 175, or 200% from the median GeneSetScore (Up hypoxia) of the population of cells at the beginning of step (i);
(h) the median GeneSetScore (Up hypoxia) of the population of cells from step (iii) is lower (for example, at least about 40, 50, 60, 70, or 80% lower) than the median GeneSetScore (Up hypoxia) of:
cells made by an otherwise similar method in which step (iii) is performed more than 26 hours after the beginning of step (i), for example, more than 5, 6, 7, 8, 9, 10, 11, or 12 days after the beginning of step (i), or
cells made by an otherwise similar method which further comprises, after step (ii) and prior to step (iii), expanding the population of cells (for example, T cells) in vitro for more than 3 days, for example, for 5, 6, 7, 8 or 9 days;
(j) the median GeneSetScore (Up autophagy) of the population of cells from step (iii) is about the same as or differs by no more than (for example, increased by no more than) about 180, 190, 200, or 210% from the median GeneSetScore (Up autophagy) of the population of cells at the beginning of step (i); or
(k) the median GeneSetScore (Up autophagy) of the population of cells from step (iii) is lower (for example, at least 20, 30, or 40% lower) than the median GeneSetScore (Up autophagy) of:
cells made by an otherwise similar method in which step (iii) is performed more than 26 hours after the beginning of step (i), for example, more than 5, 6, 7, 8, 9, 10, 11, or 12 days after the beginning of step (i), or
cells made by an otherwise similar method which further comprises, after step (ii) and prior to step (iii), expanding the population of cells (for example, T cells) in vitro for more than 3 days, for example, for 5, 6, 7, 8 or 9 days.

10. The method of any one of claims 1-9, wherein the population of cells from step (iii), after being incubated with a cell expressing an antigen recognized by the CCAR or CAR, secretes IL-2 at a higher level (for example, at least 2, 4, 6, 8, 10, 12, or 14-fold higher) than cells made by an otherwise similar method in which step (iii) is performed more than 26 hours after the beginning of step (i), for example, more than 5, 6, 7, 8, 9, 10, 11, or 12 days after the beginning of step (i), or cells made by an otherwise similar method which further comprises, after step (ii) and prior to step (iii), expanding the population of cells (for example, T cells) in vitro for more than 3 days, for example, for 5, 6, 7, 8 or 9 days, for example, as assessed using methods described in Example 8 with respect to FIGS. 29C-29D.

11. The method of any one of claims 1-10, wherein the population of cells from step (iii), after being administered in vivo, persists longer or expands at a higher level (for example, as assessed using methods described in Example 1 with respect to FIG. 4C), compared with cells made by an otherwise similar method in which step (iii) is performed more than 26 hours after the beginning of step (i), for example, more than 5, 6, 7, 8, 9, 10, 11, or 12 days after the beginning of step (i), or compared with cells made by an otherwise similar method which further comprises, after step (ii) and prior to step (iii), expanding the population of cells (for example, T cells) in vitro for more than 3 days, for example, for 5, 6, 7, 8 or 9 days.

12. The method of any one of claims 1-11, wherein the population of cells from step (iii), after being administered in vivo, shows a stronger anti-tumor activity (for example, a stronger anti-tumor activity at a low dose, for example, a dose no more than 0.15×10⁶, 0.2×10⁶, 0.25×10⁶, or 0.3×10⁶ viable cells that comprise the first or second nucleic acid molecule) than cells made by an otherwise similar method in which step (iii) is performed more than 26 hours after the beginning of step (i), for example, more than 5, 6, 7, 8, 9, 10, 11, or 12 days after the beginning of step (i), or cells made by an otherwise similar method which further comprises, after step (ii) and prior to step (iii), expanding the population of cells (for example, T cells) in vitro for more than 3 days, for example, for 5, 6, 7, 8 or 9 days.

13. The method of any one of claims 1-12, the population of cells from step (iii) are not expanded, or expanded by no more than 5, 10, 15, 20, 25, 30, 35, or 40%, for example, no more than 10%, for example, as assessed by the number of living cells, compared to the population of cells at the beginning of step (i), optionally wherein the number of living cells in the population of cells from step (iii) decreases from the number of living cells in the population of cells at the beginning of step (i).

14. The method of any one of claims 1-13, wherein the population of cells from step (iii) are not expanded, or expanded by less than 2 hours, for example, less than 1 or 1.5 hours, compared to the population of cells at the beginning of step (i).

15. The method of any one of claims 1-14, wherein steps (i) and/or (ii) are performed in cell media (for example, serum-free media) comprising IL-2, IL-15 (for example, hetIL-15 (IL15/sIL-15Ra)), IL-7, IL-21, IL-6 (for example, IL-6/sIL-6Ra), a LSD1 inhibitor, a MALT1 inhibitor, or a combination thereof.

16. The method of any one of claims 1-15, wherein steps (i) and/or (ii) are performed in serum-free cell media comprising a serum replacement.

17. The method of claim 16, wherein the serum replacement is CTS™ Immune Cell Serum Replacement (ICSR).

18. The method of any one of claims 1-17, further comprising prior to step (i):
(iv) (optionally) receiving a fresh leukapheresis product (or an alternative source of hematopoietic tissue such as a fresh whole blood product, a fresh bone marrow product, or a fresh tumor or organ biopsy or removal (for example, a fresh product from thymectomy)) from an entity, for example, a laboratory, hospital, or healthcare provider, and
(v) isolating the population of cells (for example, T cells, for example, CD8+ and/or CD4+ T cells) contacted in step (i) from a fresh leukapheresis product (or an alternative source of hematopoietic tissue such as a fresh whole blood product, a fresh bone marrow product, or a fresh tumor or organ biopsy or removal (for example, a fresh product from thymectomy)), optionally wherein:
step (iii) is performed no later than 35 hours after the beginning of step (v), for example, no later than 27, 28, 29, 30, 31, 32, 33, 34, or 35 hours after the beginning of step (v), for example, no later than 30 hours after the beginning of step (v), or
the population of cells from step (iii) are not expanded, or expanded by no more than 5, 10, 15, 20, 25, 30, 35, or 40%, for example, no more than 10%, for example, as assessed by the number of living cells, compared to the population of cells at the end of step (v).

19. The method of any one of claims 1-17, further comprising prior to step (i): receiving cryopreserved T cells isolated from a leukapheresis product (or an alternative source of hematopoietic tissue such as cryopreserved T cells isolated from whole blood, bone marrow, or tumor or organ biopsy or removal (for example, thymectomy)) from an entity, for example, a laboratory, hospital, or healthcare provider.

20. The method of any one of claims 1-17, further comprising prior to step (i):
(iv) (optionally) receiving a cryopreserved leukapheresis product (or an alternative source of hematopoietic tissue such as a cryopreserved whole blood product, a cryopreserved bone marrow product, or a cryopreserved tumor or organ biopsy or removal (for example, a cryopreserved product from thymectomy)) from an entity, for example, a laboratory, hospital, or healthcare provider, and
(v) isolating the population of cells (for example, T cells, for example, CD8+ and/or CD4+ T cells) contacted in step (i) from a cryopreserved leukapheresis product (or an alternative source of hematopoietic tissue such as a cryopreserved whole blood product, a cryopreserved bone marrow product, or a cryopreserved tumor or organ biopsy or removal (for example, a cryopreserved product from thymectomy)), optionally wherein:
step (iii) is performed no later than 35 hours after the beginning of step (v), for example, no later than 27, 28, 29, 30, 31, 32, 33, 34, or 35 hours after the beginning of step (v), for example, no later than 30 hours after the beginning of step (v), or
the population of cells from step (iii) are not expanded, or expanded by no more than 5, 10, 15, 20, 25, 30, 35, or 40%, for example, no more than 10%, for example, as assessed by the number of living cells, compared to the population of cells at the end of step (v).

21. The method of any one of claims 1-20, further comprising step (vi):
culturing a portion of the population of cells from step (iii) for at least 2, 2.5, 3, 3.5, 4, 4.5, 5, 5.5, 6, 6.5, or 7 days, for example, at least 2 days and no more than 7 days, and measuring CAR (e.g., CCAR) expression level in the portion (for example, measuring the percentage of viable, CAR-expressing cells (e.g., CCAR-expressing cells) in the portion), optionally wherein:
step (iii) comprises harvesting and freezing the population of cells (for example, T cells) and step (vi) comprises thawing a portion of the population of cells from step (iii), culturing the portion for at least 2, 2.5, 3, 3.5, 4, 4.5, 5, 5.5, 6, 6.5, or 7 days, for example, at least 2 days and no more than 7 days, and measuring CAR (e.g., CCAR) expression level in the portion (for example, measuring the percentage of viable, CAR-expressing cells (e.g., CCAR-expressing cells) in the portion).

22. A method of making a population of cells (for example, T cells) that comprise:
a first nucleic acid molecule that encodes a controllable chimeric antigen receptor (CCAR), or
a second nucleic acid molecule that encodes a chimeric antigen receptor (CAR) and a regulatory molecule,
the method comprising:
(1) contacting a population of cells (for example, T cells, for example, T cells isolated from a frozen leukapheresis product) with a cytokine chosen from IL-2, IL-7, IL-15, IL-21, IL-6, or a combination thereof,
(2) contacting the population of cells (for example, T cells) with a first nucleic acid molecule (for example, a DNA or RNA molecule) encoding a CCAR or a second nucleic acid molecule (for example, a DNA or RNA molecule) encoding a CAR and a regulatory molecule, thereby providing a population of cells (for example, T cells) comprising the first or second nucleic acid molecule, and
(3) harvesting the population of cells (for example, T cells) for storage (for example, reformulating the population of cells in cryopreservation media) or administration, wherein:
(a) step (2) is performed together with step (1) or no later than 5 hours after the beginning of step (1), for example, no later than 1, 2, 3, 4, or 5 hours after the beginning of step (1), and
step (3) is performed no later than 26 hours after the beginning of step (1), for example, no later than 22, 23, or 24 hours after the beginning of step (1), for example, no later than 24 hours after the beginning of step (1), or
(b) the population of cells from step (3) are not expanded, or expanded by no more than 5, 10, 15, 20, 25, 30, 35, or 40%, for example, no more than 10%, for example, as assessed by the number of living cells, compared to the population of cells at the beginning of step (1),
optionally wherein the first or second nucleic acid molecule in step (2) is on a viral vector, optionally wherein the first or second nucleic acid molecule in step (ii) is an RNA molecule on a viral vector, optionally wherein step (ii) comprises transducing the population of cells (for example, T cells) with a viral vector comprising the first or second nucleic acid molecule.

23. The method of claim 22, wherein step (1) comprises contacting the population of cells (for example, T cells) with IL-2.

24. The method of claim 22, wherein step (1) comprises contacting the population of cells (for example, T cells) with IL-7.

25. The method of claim 22, wherein step (1) comprises contacting the population of cells (for example, T cells) with IL-15 (for example, hetIL-15 (IL15/sIL-15Ra)).

26. The method of claim 22, wherein step (1) comprises contacting the population of cells (for example, T cells) with IL-21.

27. The method of claim 22, wherein step (1) comprises contacting the population of cells (for example, T cells) with IL-6 (for example, IL-6/sIL-6Ra).

28. The method of claim 22, wherein step (1) comprises contacting the population of cells (for example, T cells) with IL-7 and IL-15 (for example, hetIL-15 (IL15/sIL-15Ra)).

29. The method of claim 22, wherein step (1) comprises contacting the population of cells (for example, T cells) with IL-7 and IL-21.

30. The method of claim 22, wherein step (1) comprises contacting the population of cells (for example, T cells) with IL-15 (for example, hetIL-15 (IL15/sIL-15Ra)) and IL-21.

31. The method of claim 22, wherein step (1) comprises contacting the population of cells (for example, T cells) with IL-7, IL-15 (for example, hetIL-15 (IL15/sIL-15Ra)), and IL-21.

32. The method of claim 22, wherein step (1) comprises contacting the population of cells (for example, T cells) with IL-6 (for example, IL-6/sIL-6Ra) and IL-15 (for example, hetIL-15 (IL15/sIL-15Ra)).

33. The method of claim 22, wherein step (1) comprises contacting the population of cells (for example, T cells) with IL-2 and IL-6 (for example, IL-6/sIL-6Ra).

34. The method of any one of claims 22-33, wherein the population of cells from step (3) shows a higher percentage of naïve cells among cells that comprise the first or second nucleic acid molecule (for example, at least 10, 15, 20, 25, 30, 35, or 40% higher), compared with cells made by an otherwise similar method which further comprises contacting the population of cells with, for example, an anti-CD3 antibody.

35. The method of any one of claims 22-34, wherein the percentage of naïve cells, for example, naïve T cells, for example, CD45RA+ CD45RO− CCR7+ T cells, in the population of cells from step (3):
(a) is the same as or differs by no more than 5 or 10% from the percentage of naïve cells, for example, naïve T cells, for example, CD45RA+ CD45RO− CCR7+ cells, in the population of cells at the beginning of step (1), or
(b) is increased, for example, increased by at least 10 or 20%, as compared to the percentage of naïve cells, for example, naïve T cells, for example, CD45RA+ CD45RO− CCR7+ cells, in the population of cells at the beginning of step (1).

36. The method of any one of claims 22-35, wherein the population of cells from step (3) shows a higher percentage of naïve cells, for example, naïve T cells, for example, CD45RA+ CD45RO− CCR7+ T cells (for example, at least 10, 20, 30, or 40% higher), compared with cells made by an otherwise similar method in which step (3) is performed more than 26 hours after the beginning of step (1), for example, more than 5, 6, 7, 8, 9, 10, 11, or 12 days after the beginning of step (1).

37. The method of any one of claims 22-36, wherein the population of cells from step (3) shows a higher percentage of naïve cells, for example, naïve T cells, for example, CD45RA+ CD45RO− CCR7+ T cells (for example, at least 10, 20, 30, or 40% higher), compared with cells made by an otherwise similar method which further comprises, after step (2) and prior to step (3), expanding the population of cells (for example, T cells) in vitro for more than 3 days, for example, for 5, 6, 7, 8 or 9 days.

38. The method of any one of claims 22-37, wherein the population of cells from step (3), after being administered in vivo, persists longer or expands at a higher level (for example, as assessed using methods described in Example 1 with respect to FIG. 4C), compared with cells made by an otherwise similar method in which step (3) is performed more than 26 hours after the beginning of step (1), for example, more than 5, 6, 7, 8, 9, 10, 11, or 12 days after the beginning of step (1).

39. The method of any one of claims 22-38, wherein the population of cells from step (3), after being administered in vivo, persists longer or expands at a higher level (for example, as assessed using methods described in Example 1 with respect to FIG. 4C), compared with cells made by an otherwise similar method which further comprises, after step (2) and prior to step (3), expanding the population of cells (for example, T cells) in vitro for more than 3 days, for example, for 5, 6, 7, 8 or 9 days.

40. The method of any one of claims 22-39, the population of cells from step (3) are not expanded, or expanded by no more than 5, 10, 15, 20, 25, 30, 35, or 40%, for example, no more than 10%, for example, as assessed by the number of living cells, compared to the population of cells at the beginning of step (1), optionally wherein the number of living cells in the population of cells from step (3) decreases from the number of living cells in the population of cells at the beginning of step (1).

41. The method of any one of claims 22-40, wherein the population of cells from step (3) are not expanded, or expanded by less than 2 hours, for example, less than 1 or 1.5 hours, compared to the population of cells at the beginning of step (1).

42. The method of any one of claims 22-41, wherein the population of cells is not contacted in vitro with an agent that stimulates a CD3/TCR complex and/or an agent that stimulates a costimulatory molecule on the surface of the cells, or if contacted, the contacting step is less than 2 hours, for example, no more than 1 or 1.5 hours.

43. The method of claim 42, wherein the agent that stimulates a CD3/TCR complex is an agent that stimulates CD3 (for example, an anti-CD3 antibody) and wherein the agent that stimulates a costimulatory molecule is an agent that stimulates CD28, ICOS, CD27, HVEM, LIGHT, CD40, 4-1BB, OX40, DR3, GITR, CD30, TIM1, CD2, CD226, or any combination thereof, optionally wherein the agent that stimulates a CD3/TCR complex or the agent that stimulates a costimulatory molecule is chosen from an antibody (for example, a single-domain antibody (for example, a heavy chain variable domain antibody), a peptibody, a Fab fragment, or a scFv), a small molecule, or a ligand (for example, a naturally-existing, recombinant, or chimeric ligand).

44. The method of any one of claims 22-43, wherein steps (1) and/or (2) are performed in cell media comprising:
no more than 5, 4, 3, 2, 1, or 0% serum, optionally wherein steps (1) and/or (2) are performed in cell media comprising about 2% serum, or
a LSD1 inhibitor or a MALT1 inhibitor.

45. The method of any one of claims 22-44, further comprising receiving a cryopreserved leukapheresis product (or an alternative source of hematopoietic tissue such as a cryopreserved whole blood product, a cryopreserved bone marrow product, or a cryopreserved tumor or organ biopsy or removal (for example, a cryopreserved product from thymectomy)) from an entity, for example, a laboratory, hospital, or healthcare provider.

46. The method of any one of claims 1-45, wherein the population of cells at the beginning of step (i) or step (1) has been enriched for IL6R-expressing cells (for example, cells that are positive for IL6Rα and/or IL6Rβ).

47. The method of any one of claims 1-46, wherein the population of cells at the beginning of step (i) or step (1) comprises no less than 50, 60, or 70% of IL6R-expressing cells (for example, cells that are positive for IL6Rα and/or IL6Rβ).

48. The method of any one of claims 1-47, wherein steps (i) and (ii) or steps (1) and (2) are performed in cell media comprising IL-15 (for example, hetIL-15 (IL15/sIL-15Ra)).

49. The method of claim 48, wherein IL-15 increases the ability of the population of cells to expand, for example, 10, 15, 20, or 25 days later.

50. The method of claim 48, wherein IL-15 increases the percentage of IL6Rβ-expressing cells in the population of cells.

51. The method of any one of claims 1-50, wherein the CCAR or CAR comprises an antigen binding domain, a transmembrane domain, and/or an intracellular signaling domain.

52. The method of claim 51, wherein the antigen binding domain binds to an antigen chosen from:
CD19, CD20, CD22, BCMA, mesothelin, EGFRvIII, GD2, Tn antigen, sTn antigen, Tn-O-Glycopeptides, sTn-O-Glycopeptides, PSMA, CD97, TAG72, CD44v6, CEA, EPCAM, KIT, IL-13Ra2, leguman, GD3, CD171, IL-11Ra, PSCA, MAD-CT-1, MAD-CT-2, VEGFR2, LewisY, CD24, PDGFR-beta, SSEA-4, folate receptor alpha, ERBBs (for example, ERBB2), Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, NY-ESO-1, GPR20, Ly6k, OR51E2, TARP, GFRα4, or a peptide of any of these antigens presented on MHC.

53. The method of claim 51 or 52, wherein the antigen binding domain comprises a CDR, VH, VL, or scFv sequence disclosed herein, optionally wherein:
(a) the antigen binding domain binds to BCMA and comprises a CDR, VH, VL, scFv or CAR sequence disclosed in Tables 3-15, or a sequence having at least 80%, 85%, 90%, 95%, or 99% identity thereto;
(b) the antigen binding domain binds to CD19 and comprises a CDR, VH, VL, scFv or CAR sequence disclosed in Table 2, or a sequence having at least 80%, 85%, 90%, 95%, or 99% identity thereto;
(c) the antigen binding domain binds to CD20 and comprises a CDR, VH, VL, scFv or CAR sequence disclosed herein, or a sequence having at least 80%, 85%, 90%, 95%, or 99% identity thereto; or
(d) the antigen binding domain binds to CD22 and comprises a CDR, VH, VL, scFv or CAR sequence disclosed herein, or a sequence having at least 80%, 85%, 90%, 95%, or 99% identity thereto.

54. The method of any one of claims 51-53, wherein the antigen binding domain comprises a VH and a VL, wherein the VH and VL are connected by a linker, optionally wherein the linker comprises the amino acid sequence of SEQ ID NO: 63 or 104.

55. The method of any one of claims 51-54, wherein:
(a) the transmembrane domain comprises a transmembrane domain of a protein chosen from the alpha, beta or zeta chain of T-cell receptor, CD28, CD3 epsilon, CD45, CD4, CD5, CD8, CD9, CD16, CD22, CD33, CD37, CD64, CD80, CD86, CD134, CD137 and CD154,
(b) the transmembrane domain comprises a transmembrane domain of CD8,
(c) the transmembrane domain comprises the amino acid sequence of SEQ ID NO: 6, or an amino acid sequence having at least about 85%, 90%, 95%, or 99% sequence identity thereof, or
(d) the first or second nucleic acid molecule comprises a nucleic acid sequence encoding the transmembrane domain, wherein the nucleic acid sequence comprises the nucleic acid sequence of SEQ ID NO: 17, or a nucleic acid sequence having at least about 85%, 90%, 95%, or 99% sequence identity thereof.

56. The method of any one of claims 51-55, wherein the antigen binding domain is connected to the transmembrane domain by a hinge region, optionally wherein:
(a) the hinge region comprises the amino acid sequence of SEQ ID NO: 2, 3, or 4, or an amino acid sequence having at least about 85%, 90%, 95%, or 99% sequence identity thereof, or
(b) the first or second nucleic acid molecule comprises a nucleic acid sequence encoding the hinge region, wherein the nucleic acid sequence comprises the nucleic acid sequence of SEQ ID NO: 13, 14, or 15, or a nucleic acid sequence having at least about 85%, 90%, 95%, or 99% sequence identity thereof.

57. The method of any one of claims 51-56, wherein the intracellular signaling domain comprises a primary signaling domain, optionally wherein the primary signaling domain comprises a functional signaling domain derived from CD3 zeta, TCR zeta, FcR gamma, FcR beta, CD3 gamma, CD3 delta, CD3 epsilon, CD5, CD22, CD79a, CD79b, CD278 (ICOS), FcεRI, DAP10, DAP12, or CD66d, optionally wherein:
(a) the primary signaling domain comprises a functional signaling domain derived from CD3 zeta,
(b) the primary signaling domain comprises the amino acid sequence of SEQ ID NO: 9 or 10, or an amino acid sequence having at least about 85%, 90%, 95%, or 99% sequence identity thereof, or
(c) the first or second nucleic acid molecule comprises a nucleic acid sequence encoding the primary signaling domain, wherein the nucleic acid sequence comprises the nucleic acid sequence of SEQ ID NO: 20 or 21, or a nucleic acid sequence having at least about 85%, 90%, 95%, or 99% sequence identity thereof.

58. The method of any one of claims 51-57, wherein the intracellular signaling domain comprises a costimulatory signaling domain, optionally wherein the costimulatory signaling domain comprises a functional signaling domain derived from a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CD5, ICAM-1, 4-1BB (CD137), B7-H3, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD28-OX40, CD28-4-1BB, or a ligand that specifically binds with CD83, optionally wherein:
(a) the costimulatory signaling domain comprises a functional signaling domain derived from 4-1BB,
(b) the costimulatory signaling domain comprises the amino acid sequence of SEQ ID NO: 7, or an amino acid sequence having at least about 85%, 90%, 95%, or 99% sequence identity thereof, or
(c) the first or second nucleic acid molecule comprises a nucleic acid sequence encoding the costimulatory signaling domain, wherein the nucleic acid sequence comprises the nucleic acid sequence of SEQ ID NO: 18, or a nucleic acid sequence having at least about 85%, 90%, 95%, or 99% sequence identity thereof.

59. The method of any one of claims 51-58, wherein the intracellular signaling domain comprises a functional signaling domain derived from 4-1BB and a functional signaling domain derived from CD3 zeta, optionally wherein the intracellular signaling domain comprises the amino acid sequence of SEQ ID NO: 7 (or an amino acid sequence having at least about 85%, 90%, 95%, or 99% sequence identity thereof) and the amino acid sequence of SEQ ID NO: 9 or 10 (or an amino acid sequence having at least about 85%, 90%, 95%, or 99% sequence identity thereof), optionally wherein the intracellular signaling domain comprises the amino acid sequence of SEQ ID NO: 7 and the amino acid sequence of SEQ ID NO: 9 or 10.

60. The method of any one of claims 51-59, wherein the CCAR or CAR further comprises a leader sequence comprising the amino acid sequence of SEQ ID NO: 1.

61. A population of cells that comprise the first or second nucleic acid molecule (for example, autologous or allogeneic T cells or NK cells that comprise the first or second nucleic acid molecule) made by the method of any one of claims 1-60.

62. A population of cells engineered to comprise:
a first nucleic acid molecule that encodes a CCAR, or
a second nucleic acid molecule that encodes a CAR and a regulatory molecule,
said population comprising:
(a) about the same percentage of naïve cells, for example, naïve T cells, for example, CD45RO− CCR7+ T cells, as compared to the percentage of naïve cells, for example, naïve T cells, for example, CD45RO− CCR7+ cells, in the same population of cells prior to being engineered to comprise the first or second nucleic acid molecule;
(b) a change within about 5% to about 10% of naïve cells, for example, naïve T cells, for example, CD45RO− CCR7+ T cells, for example, as compared to the percentage of naïve cells, for example, naïve T cells, for example, CD45RO− CCR7+ cells, in the same population of cells prior to being engineered to comprise the first or second nucleic acid molecule;
(c) an increased percentage of naïve cells, for example, naïve T cells, for example, CD45RO− CCR7+ T cells, for example, increased by at least 1.2, 1.4, 1.6, 1.8, 2.0, 2.2, 2.4, 2.6, 2.8, or 3-fold, as compared to the percentage of naïve cells, for example, naïve T cells, for example, CD45RO− CCR7+ cells, in the same population of cells prior to being engineered to comprise the first or second nucleic acid molecule;
(d) about the same percentage of central memory cells, for example, central memory T cells, for example, CCR7+CD45RO+ T cells, as compared to the percentage of central memory cells, for example, central memory T cells, for example, CCR7+CD45RO+ T cells, in the same population of cells prior to being engineered to comprise the first or second nucleic acid molecule;
(e) a change within about 5% to about 10% of central memory cells, for example, central memory T cells, for example, CCR7+CD45RO+ T cells, as compared to the percentage of central memory cells, for example, central memory T cells, for example, CCR7+CD45RO+ T cells, in the same population of cells prior to being engineered to comprise the first or second nucleic acid molecule;
(f) a decreased percentage of central memory cells, for example, central memory T cells, for example, CCR7+CD45RO+ T cells, for example, decreased by at least 20, 25, 30, 35, 40, 45, or 50%, as compared to the percentage of central memory cells, for example, central memory T cells, for example, CCR7+CD45RO+ T cells, in the same population of cells prior to being engineered to comprise the first or second nucleic acid molecule;
(g) about the same percentage of stem memory T cells, for example, CD45RA+CD95+IL-2 receptor β+CCR7+CD62L+ T cells, as compared to the percentage of stem memory T cells, for example, CD45RA+CD95+IL-2 receptor β+CCR7+CD62L+ T cells, in the same population of cells prior to being engineered to comprise the first or second nucleic acid molecule;
(h) a change within about 5% to about 10% of stem memory T cells, for example, CD45RA+CD95+IL-2 receptor β+CCR7+CD62L+ T cells, as compared to the percentage of stem memory T cells, for example, CD45RA+CD95+IL-2 receptor β+CCR7+CD62L+ T cells, in the same population of cells prior to being engineered to comprise the first or second nucleic acid molecule; or
(i) an increased percentage of stem memory T cells, for example, CD45RA+CD95+IL-2 receptor β+CCR7+CD62L+ T cells, as compared to the percentage of stem memory T cells, for example, CD45RA+CD95+IL-2 receptor β+CCR7+CD62L+ T cells, in the same population of cells prior to being engineered to comprise the first or second nucleic acid molecule.

63. A population of cells engineered to comprise:
a first nucleic acid molecule that encodes a CCAR, or
a second nucleic acid molecule that encodes a CAR and a regulatory molecule,
wherein:
(a) the median GeneSetScore (Up TEM vs. Down TSCM) of the population of cells is about the same as or differs by no more than (for example, increased by no more than) about 25, 50, 75, 100, or 125% from the median GeneSetScore (Up TEM vs. Down TSCM) of the same population of cells prior to being engineered to comprise the first or second nucleic acid molecule;
(b) the median GeneSetScore (Up Treg vs. Down Teff) of the population of cells is about the same as or differs by no more than (for example, increased by no more than) about 25, 50, 100, 150, or 200% from the median GeneSetScore (Up Treg vs. Down Teff) of the population of cells prior to being engineered to comprise the first or second nucleic acid molecule;
(c) the median GeneSetScore (Down stemness) of the population of cells is about the same as or differs by no more than (for example, increased by no more than) about 25, 50, 100, 150, 200, or 250% from the median GeneSetScore (Down stemness) of the population of cells prior to being engineered to comprise the first or second nucleic acid molecule;
(d) the median GeneSetScore (Up hypoxia) of the population of cells is about the same as or differs by no more than (for example, increased by no more than) about 125, 150, 175, or 200% from the median GeneSetScore (Up hypoxia) of the population of cells prior to being engineered to comprise the first or second nucleic acid molecule; or
(e) the median GeneSetScore (Up autophagy) of the population of cells is about the same as or differs by no more than (for example, increased by no more than) about 180, 190, 200, or 210% from the median GeneSetScore (Up autophagy) of the population of cells prior to being engineered to comprise the first or second nucleic acid molecule.

64. The method of any one of claims 1-60 or the population of cells of any one of claims 61-63, wherein the population of cells comprise the first nucleic acid molecule that encodes a CCAR.

65. The method of claim 64 or the population of cells of claim 64, wherein the CCAR is a fusion polypeptide comprising a degradation polypeptide (e.g., a degradation polypeptide disclosed herein) and a CAR polypeptide (e.g., a CAR polypeptide disclosed herein).

66. The method of claim 65 or the population of cells of claim 65, wherein:
(i) the degradation polypeptide comprises or consists of an amino acid sequence selected from the group consisting of SEQ ID NOs: 310-315, 320-324, 337-339, 360-361, 367-369 and 374 (or a sequence having at least 85, 87, 90, 95, 97, 98, 99, or 100% identity thereto), optionally wherein the degradation polypeptide comprises or consists of the amino acid sequence of SEQ ID NO: 312;
(ii) the degradation polypeptide comprises a beta turn of IKZF1 or IKZF3 (or a sequence having at least 85, 87, 90, 95, 97, 98, 99, or 100% identity thereto), optionally wherein the degradation polypeptide comprises a beta hairpin or a beta strand of IKZF1 or IKZF3 (or a sequence having at least 85, 87, 90, 95, 97, 98, 99, or 100% identity thereto);
(iii) the degradation polypeptide comprises an alpha helix of IKZF1 or IKZF3 (or a sequence having at least 85, 87, 90, 95, 97, 98, 99, or 100% identity thereto);
(iv) the degradation polypeptide comprises, from the N-terminus to the C-terminus, a first beta strand, a beta hairpin, a second beta strand, and a first alpha helix of IKZF1 or IKZF3 (or a sequence having at least 85, 87, 90, 95, 97, 98, 99, or 100% identity thereto);
(v) the degradation polypeptide comprises, from the N-terminus to the C-terminus, a first beta strand, a beta hairpin, a second beta strand, a first alpha helix, and a second alpha helix of IKZF1 or IKZF3 (or a sequence having at least 85, 87, 90, 95, 97, 98, 99, or 100% identity thereto), optionally wherein the beta hairpin and the second alpha helix are separated by no more than 60, 50, 40, or 30 amino acid residues;
(vi) the degradation polypeptide comprises about 10 to about 95 amino acid residues, about 15 to about 90 amino acid residues, about 20 to about 85 amino acid residues, about 25 to about 80 amino acid residues, about 30 to about 75 amino acid residues, about 35 to about 70 amino acid residues, about 40 to about 65 amino acid residues, about 45 to about 65 amino acid residues, about 50 to about 65 amino acid residues, or about 55 to about 65 amino acid residues of IKZF1 or IKZF3 (or a sequence having at least 85, 87, 90, 95, 97, 98, 99, or 100% identity thereto);
(vii) the degradation polypeptide comprises at least 10 amino acids, at least 15 amino acids, at least 20 amino acids, at least 25 amino acids, at least 30 amino acids, at least 35 amino acids, at least 40 amino acids, at least 45 amino acids, at least 50 amino acids, at least 55 amino acids, at least 60 amino acids, at least 65 amino acids, at least 70 amino acids, at least 75 amino acids, at least 80 amino acids, at least 85 amino acids, at least 90 amino acids, at least 90 amino acids, or at least 95 amino acids of IKZF1 or IKZF3 (or a sequence having at least 85, 87, 90, 95, 97, 98, 99, or 100% identity thereto);
(viii) the association of the fusion polypeptide with cereblon (CRBN) in the absence of COF1 or COF2, e.g., an immunomodulatory imide drug (IMiD), e.g., lenalidomide, pomalidomide, or thalidomide, is no more than, e.g., 0.01%, 0.1%, 1%, 5%, 10%, 15%, or 20%, of the association of the fusion polypeptide with CRBN in the presence of COF1 or COF2, e.g., an IMiD, e.g., lenalidomide, pomalidomide, or thalidomide;
(ix) the ubiquitination of the fusion polypeptide in the absence of COF1 or COF2, e.g., an IMiD, e.g., lenalidomide, pomalidomide, or thalidomide, is no more than, e.g., 0.01%, 0.1%, 1%, 10%, 20%, 30%, 40%, 50%, 60%, or 70%, of the ubiquitination of the fusion polypeptide in the presence of COF1 or COF2, e.g., an IMiD, e.g., lenalidomide, pomalidomide, or thalidomide;
(x) the degradation of the fusion polypeptide in the absence of COF1 or COF2, e.g., an IMiD, e.g., lenalidomide, pomalidomide, or thalidomide, is no more than, e.g., 0.01%, 0.1%, 1%, 10%, 20%, 30%, 40%, 50%, 60%, or 70% of the degradation of the fusion polypeptide in the presence of COF1 or COF2, e.g., an IMiD, e.g., lenalidomide, pomalidomide, or thalidomide; and/or
(xi) the expression level of the fusion polypeptide in the presence of COF1 or COF2, e.g., an IMiD, e.g., lenalidomide, pomalidomide, or thalidomide, is decreased by, e.g., at least 40, 50, 60, 70, 80, 90, or 99%, as compared to the expression level of the fusion polypeptide in the absence of COF1 or COF2, e.g., an IMiD, e.g., lenalidomide, pomalidomide, or thalidomide.

67. The method of claim 65 or the population of cells of claim 65, wherein:
(i) the degradation polypeptide comprises or consists of an amino acid sequence selected from the group consisting of SEQ ID NOs: 375-377 (or a sequence having at least 85, 87, 90, 95, 97, 98, 99, or 100% identity thereto), optionally wherein the degradation polypeptide comprises or consists of the amino acid sequence of SEQ ID NO: 375;
(ii) the degradation polypeptide comprises a beta turn of IKZF2 (or a sequence having at least 85, 87, 90, 95, 97, 98, 99, or 100% identity thereto), optionally wherein the degradation polypeptide comprises a beta hairpin or a beta strand of IKZF2 (or a sequence having at least 85, 87, 90, 95, 97, 98, 99, or 100% identity thereto);
(iii) the degradation polypeptide comprises an alpha helix of IKZF2 (or a sequence having at least 85, 87, 90, 95, 97, 98, 99, or 100% identity thereto);
(iv) the degradation polypeptide comprises, from the N-terminus to the C-terminus, a first beta strand, a beta hairpin, a second beta strand, and a first alpha helix of IKZF2 (or a sequence having at least 85, 87, 90, 95, 97, 98, 99, or 100% identity thereto);
(v) the degradation polypeptide comprises, from the N-terminus to the C-terminus, a first beta strand, a beta hairpin, a second beta strand, a first alpha helix, and a second alpha helix of IKZF2 (or a sequence having at least 85, 87, 90, 95, 97, 98, 99, or 100% identity thereto), optionally wherein the beta hairpin and the second alpha helix are separated by no more than 60, 50, 40, or 30 amino acid residues;
(vi) the degradation polypeptide comprises about 10 to about 95 amino acid residues, about 15 to about 90 amino acid residues, about 20 to about 85 amino acid residues, about 25 to about 80 amino acid residues, about 30 to about 75 amino acid residues, about 35 to about 70 amino acid residues, about 40 to about 65 amino acid residues, about 45 to about 65 amino acid residues, about 50 to about 65 amino acid residues, or about 55 to about 65 amino acid residues of IKZF2 (or a sequence having at least 85, 87, 90, 95, 97, 98, 99, or 100% identity thereto);
(vii) the degradation polypeptide comprises at least 10 amino acids, at least 15 amino acids, at least 20 amino acids, at least 25 amino acids, at least 30 amino acids, at least 35 amino acids, at least 40 amino acids, at least 45 amino acids, at least 50 amino acids, at least 55 amino acids, at least 60 amino acids, at least 65 amino acids, at least 70 amino acids, at least 75 amino acids, at least 80 amino acids, at least 85 amino acids, at least 90 amino acids, at least 90 amino acids, or at least 95 amino acids of IKZF2 (or a sequence having at least 85, 87, 90, 95, 97, 98, 99, or 100% identity thereto);
(viii) the association of the fusion polypeptide with cereblon (CRBN) in the absence of COF3, e.g., Compound I-112 disclosed in Table 29, is no more than, e.g., 0.01%, 0.1%, 1%, 5%, 10%, 15%, or 20%, of the association of the fusion polypeptide with CRBN in the presence of COF3, e.g., Compound I-112 disclosed in Table 29;
(ix) the ubiquitination of the fusion polypeptide in the absence of COF3, e.g., Compound I-112 disclosed in Table 29, is no more than, e.g., 0.01%, 0.1%, 1%, 10%, 20%, 30%, 40%, 50%, 60%, or 70%, of the ubiquitination of the fusion polypeptide in the presence of COF3, e.g., Compound I-112 disclosed in Table 29;
(x) the degradation of the fusion polypeptide in the absence of COF3, e.g., Compound I-112 disclosed in Table 29, is no more than, e.g., 0.01%, 0.1%, 1%, 10%, 20%, 30%, 40%, 50%, 60%, or 70% of the degradation of the fusion polypeptide in the presence of COF3, e.g., Compound I-112 disclosed in Table 29; and/or
(viii) the expression level of the fusion polypeptide in the presence of COF3, e.g., Compound I-112 disclosed in Table 29, is decreased by, e.g., at least 40, 50, 60, 70, 80, 90, or 99%, as compared to the expression level of the fusion polypeptide in the absence of COF3, e.g., Compound I-112 disclosed in Table 29.

68. The method of any one of claims 65-67 or the population of cells of any one of claims 65-67, wherein:
(i) the degradation polypeptide is fused to the CAR polypeptide;
(ii) the degradation polypeptide and the CAR polypeptide are linked by a peptide bond;
(iii) the degradation polypeptide and the CAR polypeptide are linked by a bond other than a peptide bond;
(iv) the degradation polypeptide is linked directly to the CAR polypeptide;
(v) the degradation polypeptide is linked indirectly to the CAR polypeptide;
(vi) the degradation polypeptide and the CAR polypeptide are operatively linked via a linker, e.g., a glycine-serine linker, e.g., a linker comprising the amino acid sequence of GGGGSGGGGTGGGGSG (SEQ ID NO: 335);
(vii) the degradation polypeptide is linked to the C-terminus or N-terminus of the CAR polypeptide; or
(viii) the degradation polypeptide is at the middle of the CAR polypeptide.

69. The method of claim 64 or the population of cells of claim 64, wherein the CCAR is a fusion polypeptide comprising a degradation domain (e.g., a degradation domain disclosed herein) and a CAR polypeptide (e.g., a CAR polypeptide disclosed herein), optionally wherein the degradation domain is separated from the CAR polypeptide by a heterologous protease cleavage site, optionally wherein the CCAR comprises, from the N-terminus to the C-terminus, the degradation domain, the heterologous protease cleavage site, and the CAR polypeptide.

70. The method of claim 69 or the population of cells of claim 69, wherein:
(i) the degradation domain has a first state associated with a first level of expression of the fusion polypeptide and a second state associated with a second level of expression of the fusion polypeptide, wherein the second level is increased, e.g., by at least 2-, 3-, 4-, 5-, 10-, 20- or 30-fold over the first level in the presence of a stabilization compound, optionally wherein:
(a) in the absence of the stabilization compound, the fusion polypeptide is degraded by a cellular degradation pathway, e.g., at least 50%, 60%, 70%, 80%, 90% or greater of the fusion polypeptide is degraded;
(b) in the presence of the stabilization compound, the degradation domain assumes a conformation more resistant to cellular degradation relative to a conformation in the absence of the stabilization compound; and/or
(c) in the presence of the stabilization compound, the conformation of the fusion polypeptide is more permissive to cleavage at the heterologous protease cleavage site relative to a conformation in the absence of the stabilization compound;
(ii) the degradation domain is chosen from an estrogen receptor (ER) domain, an FKB protein (FKBP) domain, or a dihydrofolate reductase (DHFR) domain, optionally wherein:
(a) the degradation domain is an estrogen receptor (ER) domain, e.g., the degradation domain comprising the amino acid sequence of SEQ ID NO: 342 or 344, or a sequence having at least 85, 87, 90, 95, 97, 98, 99, or 100% identity thereto, optionally wherein the stabilization compound is bazedoxifene or 4-hydroxy tamoxifen (4-OHT), or a pharmaceutically acceptable salt thereof;
(b) the degradation domain is an FKB protein (FKBP) domain, e.g., the degradation domain comprising the amino acid sequence of SEQ ID NO: 346, or a sequence having at least 85, 87, 90, 95, 97, 98, 99, or 100% identity thereto, optionally wherein the stabilization compound is Shield-1, or a pharmaceutically acceptable salt thereof; or
(c) the degradation domain is a dihydrofolate reductase (DHFR) domain, e.g., the degradation domain comprising the amino acid sequence of SEQ ID NO: 347, or a sequence having at least 85, 87, 90, 95, 97, 98, 99, or 100% identity thereto, optionally wherein the stabilization compound is trimethoprim, or a pharmaceutically acceptable salt thereof.

71. The method of claim 69 or 70 or the population of cells of claim 69 or 70, wherein:
(i) the heterologous protease cleavage site is cleaved by a mammalian intracellular protease, optionally wherein:
(a) the heterologous protease cleavage site is cleaved by a protease selected from the group consisting of furin, PCSK1, PCSK5, PCSK6, PCSK7, cathepsin B, Granzyme B, Factor XA, Enterokinase, genenase, sortase, precission protease, thrombin, TEV protease, and elastase 1;
(b) the heterologous protease cleavage site comprises a sequence having a cleavage motif selected from the group consisting of RX(K/R)R consensus motif (X can be any amino acid; SEQ ID NO: 348), RXXX[KR]R consensus motif (X can be any amino acid; SEQ ID NO: 349), RRX consensus motif (SEQ ID NO : 350), I-E-P-D-X consensus motif (SEQ ID NO: 351), Ile-Glu/Asp-Gly-Arg (SEQ ID NO : 352), Asp-Asp-Asp-Asp-Lys (SEQ ID NO: 353), Pro-Gly-Ala-Ala-His-Tyr (SEQ ID NO: 354), LPXTG/A consensus motif (SEQ ID NO: 355), Leu-Glu-Val-Phe-Gln-Gly-Pro (SEQ ID NO: 356), Leu-Val-Pro-Arg-Gly-Ser (SEQ ID NO: 357), E-N-L-Y-F-Q-G (SEQ ID NO: 358), and [AGSV]-X (X can be any amino acid; SEQ ID NO: 359); or
(c) the heterologous protease cleavage site comprises a furin cleavage site selected from the group consisting of RTKR (SEQ ID NO: 378); GTGAEDPRPSRKRRSLGDVG (SEQ ID NO: 379); GTGAEDPRPSRKRR (SEQ ID NO: 381); LQWLEQQVAKRRTKR (SEQ ID NO: 383); GTGAEDPRPSRKRRSLGG (SEQ ID NO: 385); GTGAEDPRPSRKRRSLG (SEQ ID NO: 387); SLNLTESHNSRKKR (SEQ ID NO: 389); CKINGYPKRGRKRR (SEQ ID NO: 391); and SARNRQKR (SEQ ID NO: 336); or
(iii) the heterologous protease cleavage site is cleaved by a mammalian extracellular protease, optionally wherein:
(a) the heterologous protease cleavage site is cleaved by a protease selected from the group consisting of Factor XA, Enterokinase, genenase, sortase, precission protease, thrombin, TEV protease, and elastase 1; or
(b) the heterologous protease cleavage site comprises an amino acid sequence selected from the group consisting of Ile-Glu/Asp-Gly-Arg (SEQ IDNO : 352), Asp-Asp-Asp-Asp-Lys (SEQ ID NO: 353), Pro-Gly-Ala-Ala-His-Tyr (SEQ ID NO: 354), LPXTG/A consensus motif (SEQ ID NO: 355), Leu-Glu-Val-Phe-Gln-Gly-Pro (SEQ ID NO: 356), Leu-Val-Pro-Arg-Gly-Ser (SEQ ID NO: 357), E-N-L-Y-F-Q-G (SEQ ID NO: 358), and [AGSV]-X (X can be any amino acid; SEQ ID NO: 359).

72. The method of claim 64 or the population of cells of claim 64, wherein the CCAR is a regulatable CAR (RCAR) (e.g., an RCAR disclosed herein).

73. The method of claim 72 or the population of cells of claim 72, wherein the RCAR comprises:
(i) an intracellular signaling member comprising: an intracellular signaling domain, e.g., a primary intracellular signaling domain, and a first switch domain;
(ii) an antigen binding member comprising: an antigen binding domain and a second switch domain; and
(iii) a transmembrane domain, optionally wherein the transmembrane domain can be disposed on the intracellular signaling member and/or the antigen binding member.

74. The method of claim 72 or the population of cells of claim 72, wherein the RCAR comprises:
(i) an intracellular signaling member comprising: an intracellular signaling domain, e.g., a primary intracellular signaling domain, and a first switch domain;
(ii) an inhibitory extracellular domain member comprising: an inhibitory extracellular domain (e.g., an inhibitory extracellular domain comprising an extracellular domain of B7-H1, B7-1, CD160, P1H, 2B4, PD1, TIM3, CEACAM, LAG3, TIGIT, CTLA-4, BTLA, LAIR1, or TGF-beta receptor, or a sequence having at least 85, 87, 90, 95, 97, 98, 99, or 100% identity thereto), and a second switch domain; and
(iii) a transmembrane domain, optionally wherein the transmembrane domain can be disposed on the intracellular signaling member and/or the inhibitory extracellular domain member.

75. The method of claim 72 or the population of cells of claim 72, wherein the RCAR comprises:
(i) an intracellular signaling member comprising: an intracellular signaling domain, e.g., a primary intracellular signaling domain, and a first switch domain;
(ii) a costimulatory extracellular domain member comprising: a costimulatory extracellular domain (e.g., a costimulatory extracellular domain comprising an extracellular domain of ICOS, CD28, VEM, LIGHT, CD40L, 4-1BB, OX40, DR3, GITR, CD30, TIM1, SLAM, CD2, or CD226, or a sequence having at least 85, 87, 90, 95, 97, 98, 99, or 100% identity thereto), and a second switch domain; and
(iii) a transmembrane domain, optionally wherein the transmembrane domain can be disposed on the intracellular signaling member and/or the costimulatory extracellular domain member.

76. The method of any one of claims 73-75 or the population of cells of any one of claims 73-75, wherein the first and second switch domains can form a dimerization switch, e.g., in the presence of a dimerization molecule, optionally wherein:
(i) the dimerization switch is an intracellular dimerization switch or an extracellular dimerization switch;
(ii) the dimerization switch is a homodimerization switch or a heterodimerization switch;
(iii) the dimerization switch comprises a FKBP-FRB based switch, e.g., a dimerization switch comprising a switch domain comprising a FRB binding fragment or analog of FKBP and a switch domain comprising a FKBP binding fragment or analog of FRB, optionally wherein the FKBP binding fragment or analog of FRB comprises one or more mutations disclosed herein (e.g., one or more mutations chosen from an E2032 mutation, a T2098 mutation, or an E2032 and a T2098 mutation), optionally wherein the dimerization molecule is an mTOR inhibitor, e.g., a rapamycin analogue, e.g., RAD001; and/or
(iv) the antigen binding domain binds to a target antigen but does not promote an immune effector response of a T cell, until the dimerization molecule is present.

77. The method of any one of claims 73-76 or the population of cells of any one of claims 73-76, wherein:
(i) the intracellular signaling member comprises a primary intracellular signaling domain, e.g., a primary intracellular signaling domain disclosed herein, e.g., a CD3zeta domain;
(ii) the intracellular signaling member comprises a costimulatory signaling domain, e.g., a costimulatory signaling domain disclosed herein, e.g., a 4-1BB domain or a CD28 domain;
(iii) the antigen binding member does not comprise a primary intracellular signaling domain, e.g., the antigen binding member comprises a costimulatory signaling domain and does not comprise a primary intracellular signaling domain;
(iv) the inhibitory extracellular domain member does not comprise a primary intracellular signaling domain, e.g., the inhibitory extracellular domain member comprises a costimulatory signaling domain and does not comprise a primary intracellular signaling domain; and/or
(v) the costimulatory extracellular domain member does not comprise a primary intracellular signaling domain, e.g., the costimulatory extracellular domain member comprises a costimulatory signaling domain and does not comprise a primary intracellular signaling domain.

78. The method of any one of claims 1-60 or the population of cells of any one of claims 61-63, wherein the population of cells comprise the second nucleic acid molecule that encodes a CAR and a regulatory molecule.

79. The method of claim 78 or the population of cells of claim 78, wherein the second nucleic acid molecule comprises a nucleic acid sequence encoding the CAR and a nucleic acid sequence encoding the regulatory molecule, optionally wherein the nucleic acid sequence encoding the CAR and the nucleic acid sequence encoding the regulatory molecule are:
(i) disposed on a single nucleic acid molecule, e.g., wherein the nucleic acid sequence encoding the CAR and the nucleic acid sequence encoding the regulatory molecule are separated by a nucleic acid sequence encoding a self-cleavage site; or
(ii) disposed on separate nucleic acid molecules.

80. The method of claim 78 or 79 or the population of cells of claim 78 or 79, wherein the regulatory molecule comprises a chimeric protein comprising (i) a multimeric ligand binding region and (ii) a caspase 9 molecule.

81. The method of claim 80 or the population of cells of claim 80, wherein the caspase 9 molecule is a truncated caspase 9, optionally wherein the caspase 9 molecule lacks the caspase recruitment domain.

82. The method of claim 80 or 81 or the population of cells of claim 80 or 81, wherein the multimeric ligand binding region is selected from the group consisting of FKBP, cyclophilin receptor, steroid receptor, tetracycline receptor, heavy chain antibody subunit, light chain antibody subunit, single chain antibodies comprised of heavy and light chain variable regions in tandem separated by a flexible linker domain, and mutated sequences thereof, optionally wherein the multimeric ligand binding region is an FKBP12 region.

83. The method of claim 78 or 79 or the population of cells of claim 78 or 79, wherein the regulatory molecule comprises a truncated epidermal growth factor receptor (EGFRt).

84. The method of claim 83 or the population of cells of claim 83, wherein the EGFRt has 1, 2, 3, 4, or all of the following properties:
(i) the EGFRt comprises one or both of an EGFR Domain III and an EGFR Domain IV;
(ii) the EGFRt does not comprise 1, 2, 3, or all of: an EGFR Domain I, an EGFR Domain II, an EGFR juxtamembrane domain, and an EGFR tyrosine kinase domain;
(iii) the EGFRt does not mediate signaling or trafficking;
(iv) the EGFRt does not bind an endogenous EGFR ligand, e.g., epidermal growth factor (EGF); and
(v) the EGFRt binds to an anti-EGFR-antibody molecule (e.g., cetuximab, matuzumab, necitumumab and panitumumab), an EGFR-specific siRNA, or a small molecule that targets EGFR.

85. A pharmaceutical composition comprising the population of cells of any one of claims 61-84 and a pharmaceutically acceptable carrier.

86. A method of increasing an immune response in a subject, comprising administering the population of cells of any one of claims 61-84 or the pharmaceutical composition of claim 85 to the subject, thereby increasing an immune response in the subject.

87. A method of treating a cancer in a subject, comprising administering the population of cells of any one of claims 61-84 or the pharmaceutical composition of claim 85 to the subject, thereby treating the cancer in the subject.

88. The method of claim 87, wherein the cancer is a solid cancer, for example, chosen from: one or more of mesothelioma, malignant pleural mesothelioma, non-small cell lung cancer, small cell lung cancer, squamous cell lung cancer, large cell lung cancer, pancreatic cancer, pancreatic ductal adenocarcinoma, esophageal adenocarcinoma , breast cancer, glioblastoma, ovarian cancer, colorectal cancer, prostate cancer, cervical cancer, skin cancer, melanoma, renal cancer, liver cancer, brain cancer, thymoma, sarcoma, carcinoma, uterine cancer, kidney cancer, gastrointestinal cancer, urothelial cancer, pharynx cancer, head and neck cancer, rectal cancer, esophagus cancer, or bladder cancer, or a metastasis thereof.

89. The method of claim 87, wherein the cancer is a liquid cancer, for example, chosen from: chronic lymphocytic leukemia (CLL), mantle cell lymphoma (MCL), multiple myeloma, acute lymphoid leukemia (ALL), Hodgkin lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitts lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+ large B-cell lymphoma, large B-cell lymphoma arising in HHV8-associated multicentric Castleman disease, primary effusion lymphoma, B-cell lymphoma, acute myeloid leukemia (AML), or unclassifiable lymphoma.

90. The method of any one of claims 86-89, further comprising administering a second therapeutic agent to the subject.

91. The method of any one of claims 86-90, wherein the population of cells is administered at a dose determined based on the percentage of CAR-expressing cells (e.g., CCAR-expressing cells) measured in claim 21.

92. The method of any one of claims 86-91, further comprising, after the administration of the population of cells or the pharmaceutical composition:
administering to the subject an effective amount of IMiD (e.g., thalidomide and derivatives thereof, e.g., lenalidomide, pomalidomide, and thalidomide) or Compound I-112, optionally wherein:
a) the subject has developed, is developing, or is anticipated to develop an adverse reaction after the administration of the population of cells or the pharmaceutical composition,
b) the administration of IMiD or Compound I-112 is in response to an occurrence of an adverse reaction in the subject, or in response to an anticipation of an occurrence of an adverse reaction in the subject, and/or
c) the administration of IMiD or Compound I-112 reduces or prevents an adverse effect, optionally wherein the population of cells is the population of cells of any one of claims 65-68.

93. A method of treating a cancer in a subject, comprising:
i) contacting the population of cells of any one of claims 65-68 with IMiD (e.g., thalidomide and derivatives thereof, e.g., lenalidomide, pomalidomide, and thalidomide) or Compound I-112 ex vivo, optionally wherein:
in the presence of IMiD or Compound I-112, the expression level of the CCAR is decreased, e.g., by at least about 1, 2, 3, 4, 5, 6, 7, 8, 9, 10, 20, 30, 40, 50, 60, 70, 80, 90, or 100 percent, relative to the expression level of the CCAR before the population of cells are contacted with IMiD or Compound I-112 ex vivo, and
ii) administering to the subject an effective amount of the population of cells, optionally wherein the method further comprises after step i) and prior to step ii):
reducing the amount of IMiD or Compound I-112 contacting the population of cells, e.g., inside and/or surrounding the population of cells,
thereby treating the cancer.

94. The method of claim 93, further comprising after step ii):
iii) administering to the subject an effective amount of IMiD or Compound I-112, optionally wherein the administration of IMiD or Compound I-112 decreases, e.g., by at least about 1, 2, 3, 4, 5, 6, 7, 8, 9, 10, 20, 30, 40, 50, 60, 70, 80, 90, or 100 percent, the expression level of the CCAR relative to the expression level of the CCAR after step ii) and prior to step iii), optionally wherein:
a) the subject has developed, is developing, or is anticipated to develop an adverse reaction,
b) the administration of IMiD or Compound I-112 is in response to an occurrence of an adverse reaction in the subject, or in response to an anticipation of an occurrence of an adverse reaction in the subject, and/or
c) the administration of IMiD or Compound I-112 reduces or prevents an adverse effect.

95. The method of claim 94, further comprising after step iii):
iv) discontinuing the administration of IMiD or Compound I-112, optionally wherein discontinuing the administration of IMiD or Compound I-112 increases, e.g., by at least about 1.5-, 2-, 3-, 4-, 5-, 10-, 20-, 30-, 40-, or 50-fold, the expression level of the CCAR relative to the expression level of the CCAR after step iii) and prior to step iv) (e.g., wherein discontinuing the administration of IMiD or Compound I-112 restores the expression level of the CCAR to the expression level after step ii) and prior to step iii)), optionally wherein:
a) the subject has relapsed, is relapsing, or is anticipated to relapse,
b) the discontinuation of the administration of IMiD or Compound I-112 is in response to a tumor relapse in the subject, or in response to an anticipation of a relapse in the subject, and/or
c) the discontinuation of the administration of IMiD or Compound I-112 treats or prevents a tumor relapse.

96. The method of claim 95, further comprising after step iv):
v) repeating step iii) and/or iv),
thereby treating the cancer.

97. A method of treating a cancer in a subject, comprising:
i) administering to the subject an effective amount of the population of cells of any one of claims 65-68, optionally wherein the population of cells are contacted with IMiD (e.g., thalidomide and derivatives thereof, e.g., lenalidomide, pomalidomide, and thalidomide) or Compound I-112 ex vivo before administration, optionally wherein:
in the presence of IMiD or Compound I-112, the expression level of the CCAR is decreased, e.g., by at least about 1, 2, 3, 4, 5, 6, 7, 8, 9, 10, 20, 30, 40, 50, 60, 70, 80, 90, or 100 percent, relative to the expression level of the CCAR before the population of cells are contacted with IMiD or Compound I-112 ex vivo, optionally wherein after the population of cells are contacted with IMiD or Compound I-112 ex vivo and before the population of cells are administered to the subject, the amount of IMiD or Compound I-112 contacting the population of cells, e.g., inside and/or surrounding the population of cells, is reduced,
thereby treating the cancer.

98. The method of claim 97, wherein the population of cells are not contacted with IMiD or Compound I-112 ex vivo before administration.

99. The method of claim 97 or 98, further comprising after step i):
ii) administering to the subject an effective amount of IMiD or Compound I-112, optionally wherein the administration of IMiD or Compound I-112 decreases, e.g., by at least about 1, 2, 3, 4, 5, 6, 7, 8, 9, 10, 20, 30, 40, 50, 60, 70, 80, 90, or 100 percent, the expression level of the CCAR relative to the expression level of the CCAR after step i) and prior to step ii), optionally wherein:
a) the subject has developed, is developing, or is anticipated to develop an adverse reaction,
b) the administration of IMiD or Compound I-112 is in response to an occurrence of an adverse reaction in the subject, or in response to an anticipation of an occurrence of an adverse reaction in the subject, and/or
c) the administration of IMiD or Compound I-112 reduces or prevents an adverse effect.

100. The method of claim 99, further comprising after step ii):
iii) discontinuing the administration of IMiD or Compound I-112, optionally wherein discontinuing the administration of IMiD or Compound I-112 increases, e.g., by at least about 1.5-, 2-, 3-, 4-, 5-, 10-, 20-, 30-, 40-, or 50-fold, the expression level of the CCAR relative to the expression level of the CCAR after step ii) and prior to step iii) (e.g., wherein discontinuing the administration of IMiD or Compound I-112 restores the expression level of the CCAR to the expression level after step i) and prior to step ii)), optionally wherein:
a) the subject has relapsed, is relapsing, or is anticipated to relapse,
b) the discontinuation of the administration of IMiD or Compound I-112 is in response to a tumor relapse in the subject, or in response to an anticipation of a relapse in the subject, and/or
c) the discontinuation of the administration of IMiD or Compound I-112 treats or prevents a tumor relapse.

101. The method of claim 100, further comprising after step iii):
iv) repeating step ii) and/or iii),
thereby treating the cancer.

102. A method of treating a cancer in a subject, comprising:
i) administering an effective amount of IMiD (e.g., thalidomide and derivatives thereof, e.g., lenalidomide, pomalidomide, and thalidomide) or Compound I-112 to the subject, wherein the subject comprises the population of cells of any one of claims 65-68, optionally wherein the administration of IMiD or Compound I-112 decreases, e.g., by at least about 1, 2, 3, 4, 5, 6, 7, 8, 9, 10, 20, 30, 40, 50, 60, 70, 80, 90, or 100 percent, the expression level of the CCAR relative to the expression level of the CCAR before the administration of IMiD or Compound I-112, optionally wherein:
a) the subject has developed, is developing, or is anticipated to develop an adverse reaction,
b) the administration of IMiD or Compound I-112 is in response to an occurrence of an adverse reaction in the subject, or in response to an anticipation of an occurrence of an adverse reaction in the subject, and/or
c) the administration of IMiD or Compound I-112 reduces or prevents an adverse effect.

103. The method of claim 102, further comprising after step i):
ii) discontinuing the administration of IMiD or Compound I-112, optionally wherein discontinuing the administration of IMiD or Compound I-112 increases, e.g., by at least about 1.5-, 2-, 3-, 4-, 5-, 10-, 20-, 30-, 40-, or 50-fold, the expression level of the CCAR relative to the expression level of the CCAR after step i) and prior to step ii) (e.g., wherein discontinuing the administration of IMiD or Compound I-112 restores the expression level of the CCAR to the expression level before the administration of IMiD or Compound I-112), optionally wherein:
a) the subject has relapsed, is relapsing, or is anticipated to relapse,
b) the discontinuation of the administration of IMiD or Compound I-112 is in response to a tumor relapse in the subject, or in response to an anticipation of a relapse in the subject, and/or
c) the discontinuation of the administration of IMiD or Compound I-112 treats or prevents a tumor relapse.

104. The method of claim 103, further comprising after step ii):
iii) repeating step i) and/or ii),
thereby treating the cancer.

105. A method of treating a cancer in a subject, comprising:
i) administering to the subject:
(1) a stabilization compound, and
(2) an effective amount of the population of cells of any one of claims 69-71, optionally wherein:
the expression level of the CCAR in the presence of the stabilization compound is e.g., at least about 1.5-, 2-, 3-, 4-, 5-, 10-, 20-, 30-, 40-, or 50-fold, higher than the expression level of the CCAR in the absence of the stabilization compound,
thereby treating the cancer.

106. The method of claim 105, further comprising after step i):
ii) discontinuing the administration of the stabilization compound, optionally wherein discontinuing the administration of the stabilization compound reduces, e.g., at least about 1.5-, 2-, 3-, 4-, 5-, 10-, 20-, 30-, 40-, or 50-fold, the expression level of the CCAR relative to the expression of the CCAR after step i) and prior to step ii), optionally wherein:
a) the subject responded to the treatment of step i) (e.g., the subject has a complete response to the treatment of step i), the subject shows a shrinkage in tumor mass, the subject shows a decrease in tumor cells, or the treatment of step i) is effective in the subject), and/or
b) the discontinuation of the administration of the stabilization compound is in response to a response of the subject to the treatment of step i) (e.g., the subject has a complete response to the treatment of step i), the subject shows a shrinkage in tumor mass, the subject shows a decrease in tumor cells, or the treatment of step i) is effective in the subject).

107. The method of claim 105, further comprising after step i):
iii) discontinuing the administration of the stabilization compound, optionally wherein discontinuing the administration of the stabilization compound reduces, e.g., at least about 1.5-, 2-, 3-, 4-, 5-, 10-, 20-, 30-, 40-, or 50-fold, the expression level of the CCAR relative to the expression of the CCAR after step i) and prior to step ii), optionally wherein:
a) the subject has developed, is developing, or is anticipated to develop an adverse reaction,
b) the discontinuation of the administration of the stabilization compound is in response to an occurrence of an adverse reaction in the subject, or in response to an anticipation of an occurrence of an adverse reaction in the subject, and/or
c) the discontinuation of the administration of the stabilization compound reduces or prevents an adverse effect.

108. The method of claim 106 or 107, further comprising after step ii) or iii):
iv) administering an effective amount of a stabilization compound, optionally wherein the administration of the stabilization compound increases, e.g., by at least about 1.5-, 2-, 3-, 4-, 5-, 10-, 20-, 30-, 40-, or 50-fold, the expression level of the CCAR relative to the expression level of the CCAR after step ii) or iii) and prior to step iv), optionally wherein:
a) the subject has relapsed, is relapsing, or is anticipated to relapse,
b) the administration of the stabilization compound is in response to a tumor relapse in the subject, or in response to an anticipation of a relapse in the subject, and/or
c) the administration of the stabilization compound treats or prevents a tumor relapse.

109. The method of claim 108, further comprising after step iv):
v) repeating step ii), iii), or iv),
thereby treating the cancer.

110. The method of any one of claims 105-109, further comprising prior to step i):
vi) contacting the population of cells with a stabilization compound ex vivo, optionally wherein the expression level of the CCAR in the presence of the stabilization compound is, e.g., at least about 1.5-, 2-, 3-, 4-, 5-, 10-, 20-, 30-, 40-, or 50-fold, higher than the expression level of the CCAR in the absence of the stabilization compound.

111. The method of any one of claims 105-109, wherein the population of cells are not contacted with the stabilization compound ex vivo before administration.

112. The population of cells of any one of claims 61-84 or the pharmaceutical composition of claim 85 for use in a method of increasing an immune response in a subject, said method comprising administering to the subject an effective amount of the population of cells or an effective amount of the pharmaceutical composition.

113. The population of cells of any one of claims 61-84 or the pharmaceutical composition of claim 85 for use in a method of treating a cancer in a subject, said method comprising administering to the subject an effective amount of the population of cells or an effective amount of the pharmaceutical composition.


SEQUENCE LISTING (420 sequences)

SEQ ID NO: 1 - protein, 21 aa, Artificial Sequence
  source: /note=“Description of Artificial Sequence: Synthetic peptide”
MALPVTALLLPLALLLHAARP

SEQ ID NO: 2 - protein, 45 aa, Artificial Sequence
  source: /note=“Description of Artificial Sequence: Synthetic polypeptide”
TTTPAPRPPTPAPTIASQPLSLRPEACRPAAGGAVHTRGLDFACD

SEQ ID NO: 3 - protein, 230 aa, Artificial Sequence
  source: /note=“Description of Artificial Sequence: Synthetic polypeptide”
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 4 - protein, 282 aa, Artificial Sequence
  source: /note=“Description of Artificial Sequence: Synthetic polypeptide”
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 5 - protein, 10 aa, Artificial Sequence
  source: /note=“Description of Artificial Sequence: Synthetic peptide”
GGGGSGGGGS

SEQ ID NO: 6 - protein, 24 aa, Artificial Sequence
  source: /note=“Description of Artificial Sequence: Synthetic peptide”
IYIWAPLAGTCGVLLLSLVITLYC

SEQ ID NO: 7 - protein, 42 aa, Artificial Sequence
  source: /note=“Description of Artificial Sequence: Synthetic polypeptide”
KRGRKKLLYIFKQPFMRPVQTTQEEDGCSCRFPEEEEGGCEL

SEQ ID NO: 8 - protein, 48 aa, Artificial Sequence
  source: /note=“Description of Artificial Sequence: Synthetic polypeptide”
QRRKYRSNKGESPVEPAEPCRYSCPREEEGSTIPIQEDYRKPEPACSP

SEQ ID NO: 9 - protein, 112 aa, Artificial Sequence
  source: /note=“Description of Artificial Sequence: Synthetic polypeptide”
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 1184 bp, Artificial Sequence
  source: /note=“Description of Artificial Sequence: Synthetic polynucleotide”
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 12 - DNA, 63 bp, Artificial Sequence
  source: /note=“Description of Artificial Sequence: Synthetic oligonucleotide”
atggccctgcctgtgacagccctgctgctgcctctggctctgctgctgcatgccgctagaccc

SEQ ID NO: 13 - DNA, 135 bp, Artificial Sequence
  source: /note=“Description of Artificial Sequence: Synthetic polynucleotide”
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 14 - DNA, 690 bp, Artificial Sequence
  source: /note=“Description of Artificial Sequence: Synthetic polynucleotide”
gagagcaagtacggccctccctgccccccttgccctgcccccgagttcctgggcggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttcaatagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaaccaggtgtccctgacctgcctggtgaagggcttctaccccagcgacatcgccgtggagtgggagagcaacggccagcccgagaacaactacaagaccaccccccctgtgctggacagcgacggcagcttcttcctgtacagccggctgaccgtggacaagagccggtggcaggagggcaacgtctttagctgctccgtgatgcacgaggccctgcacaaccactacacccagaagagcctgagcctgtccctgggcaagatg

SEQ ID NO: 15 - DNA, 847 bp, Artificial Sequence
  source: /note=“Description of Artificial Sequence: Synthetic polynucleotide”
aggtggcccgaaagtcccaaggcccaggcatctagtgttcctactgcacagccccaggcagaaggcagcctagccaaagctactactgcacctgccactacgcgcaatactggccgtggcggggaggagaagaaaaaggagaaagagaaagaagaacaggaagagagggagaccaagacccctgaatgtccatcccatacccagccgctgggcgtctatctcttgactcccgcagtacaggacttgtggcttagagataaggccacctttacatgtttcgtcgtgggctctgacctgaaggatgcccatttgacttgggaggttgccggaaaggtacccacagggggggttgaggaagggttgctggagcgccattccaatggctctcagagccagcactcaagactcacccttccgagatccctgtggaacgccgggacctctgtcacatgtactctaaatcatcctagcctgcccccacagcgtctgatggcccttagagagccagccgcccaggcaccagttaagcttagcctgaatctgctcgccagtagtgatcccccagaggccgccagctggctcttatgcgaagtgtccggctttagcccgcccaacatcttgctcatgtggctggaggaccagcgagaagtgaacaccagcggcttcgctccagcccggcccccaccccagccgggttctaccacattctgggcctggagtgtcttaagggtcccagcaccacctagcccccagccagccacatacacctgtgttgtgtcccatgaagatagcaggaccctgctaaatgcttctaggagtctggaggtttcctacgtgactgaccatt

SEQ ID NO: 16 - DNA, 30 bp, Artificial Sequence
  source: /note=“Description of Artificial Sequence: Synthetic oligonucleotide”
ggtggcggaggttctggaggtggaggttcc

SEQ ID NO: 17 - DNA, 72 bp, Artificial Sequence
  source: /note=“Description of Artificial Sequence: Synthetic oligonucleotide”
atctacatctgggcgcccttggccgggacttgtggggtccttctcctgtcactggttatcaccctttactgc

SEQ ID NO: 18 - DNA, 126 bp, Artificial Sequence
  source: /note=“Description of Artificial Sequence: Synthetic polynucleotide”
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 19 - DNA, 123 bp, Artificial Sequence
  source: /note=“Description of Artificial Sequence: Synthetic polynucleotide”
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 20 - DNA, 336 bp, Artificial Sequence
  source: /note=“Description of Artificial Sequence: Synthetic polynucleotide”
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 21 - DNA, 336 bp, Homo sapiens
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 22 - protein, 373 aa, Artificial Sequence
  source: /note=“Description of Artificial Sequence: Synthetic polypeptide”
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - DNA, 1182 bp, Artificial Sequence
  source: /note=“Description of Artificial Sequence: Synthetic polynucleotide”
atggccctccctgtcactgccctgcttctccccctcgcactcctgctccacgccgctagaccacccggatggtttctggactctccggatcgcccgtggaatcccccaaccttctcaccggcactcttggttgtgactgagggcgataatgcgaccttcacgtgctcgttctccaacacctccgaatcattcgtgctgaactggtaccgcatgagcccgtcaaaccagaccgacaagctcgccgcgtttccggaagatcggtcgcaaccgggacaggattgtcggttccgcgtgactcaactgccgaatggcagagacttccacatgagcgtggtccgcgctaggcgaaacgactccgggacctacctgtgcggagccatctcgctggcgcctaaggcccaaatcaaagagagcttgagggccgaactgagagtgaccgagcgcagagctgaggtgccaactgcacatccatccccatcgcctcggcctgcggggcagtttcagaccctggtcacgaccactccggcgccgcgcccaccgactccggccccaactatcgcgagccagcccctgtcgctgaggccggaagcatgccgccctgccgccggaggtgctgtgcatacccggggattggacttcgcatgcgacatctacatttgggctcctctcgccggaacttgtggcgtgctccttctgtccctggtcatcaccctgtactgcaagcggggtcggaaaaagcttctgtacattttcaagcagcccttcatgaggcccgtgcaaaccacccaggaggaggacggttgctcctgccggttccccgaagaggaagaaggaggttgcgagctgcgcgtgaagttctcccggagcgccgacgcccccgcctataagcagggccagaaccagctgtacaacgaactgaacctgggacggcgggaagagtacgatgtgctggacaagcggcgcggccgggaccccgaaatgggcgggaagcctagaagaaagaaccctcaggaaggcctgtataacgagctgcagaaggacaagatggccgaggcctactccgaaattgggatgaagggagagcggcggaggggaaaggggcacgacggcctgtaccaaggactgtccaccgccaccaaggacacatacgatgccctgcacatgcaggcccttccccctcgc

SEQ ID NO: 24 - protein, 394 aa, Artificial Sequence
  source: /note=“Description of Artificial Sequence: Synthetic polypeptide”
MALPVTALLLPLALLLHAARPPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 5 aa, Artificial Sequence
  source: /note=“Description of Artificial Sequence: Synthetic peptide”
  source: /note=“See specification as filed for detailed description of substitutions and preferred embodiments”
GGGGS

SEQ ID NO: 26 - protein, 30 aa, Artificial Sequence
  source: /note=“Description of Artificial Sequence: Synthetic polypeptide”
  SITE (1)..(30): /note=“This sequence may encompass 1-6 ′Gly Gly Gly Gly Ser′ repeating units”
GGGGSGGGGSGGGGSGGGGSGGGGSGGGGS

SEQ ID NO: 27 - protein, 20 aa, Artificial Sequence
  source: /note=“Description of Artificial Sequence: Synthetic peptide”
GGGGSGGGGSGGGGSGGGGS

SEQ ID NO: 28 - protein, 15 aa, Artificial Sequence
  source: /note=“Description of Artificial Sequence: Synthetic peptide”
GGGGSGGGGSGGGGS

SEQ ID NO: 29 - protein, 4 aa, Artificial Sequence
  source: /note=“Description of Artificial Sequence: Synthetic peptide”
GGGS

SEQ ID NO: 30 - RNA, 5000 nt, Artificial Sequence
  source: /note=“Description of Artificial Sequence: Synthetic polynucleotide”
  misc_feature (1)..(5000): /note=“This sequence may encompass 50-5000 nucleotides”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 31 - DNA, 100 bp, Artificial Sequence
  source: /note=“Description of Artificial Sequence: Synthetic polynucleotide”
tttttttttttttttttttttttttttttttttttttttttttttttttttttttttttttttttttttttttttttttttttttttttttttttttttt

SEQ ID NO: 32 - DNA, 5000 bp, Artificial Sequence
  source: /note=“Description of Artificial Sequence: Synthetic polynucleotide”
  misc_feature (1)..(5000): /note=“This sequence may encompass 50-5000 nucleotides”
tttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttt

SEQ ID NO: 33 - RNA, 5000 nt, Artificial Sequence; identical to SEQ ID NO: 30
  source: /note=“Description of Artificial Sequence: Synthetic polynucleotide”
  misc_feature (1)..(5000): /note=“This sequence may encompass 100-5000 nucleotides”

SEQ ID NO: 34 - RNA, 400 nt, Artificial Sequence
  source: /note=“Description of Artificial Sequence: Synthetic polynucleotide”
  misc_feature (1)..(400): /note=“This sequence may encompass 100-400 nucleotides”
  source: /note=“See specification as filed for detailed description of substitutions and preferred embodiments”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 35 - RNA, 2000 nt, Artificial Sequence
  source: /note=“Description of Artificial Sequence: Synthetic polynucleotide”
  misc_feature (1)..(2000): /note=“This sequence may encompass 50-2000 nucleotides”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 36 - protein, 41 aa, Artificial Sequence
  source: /note=“Description of Artificial Sequence: Synthetic polypeptide”
RSKRSRLLHSDYMNMTPRRPGPTRKHYQPYAPPRDFAAYRS

SEQ ID NO: 37 - DNA, 123 bp, Artificial Sequence; identical to SEQ ID NO: 19
  source: /note=“Description of Artificial Sequence: Synthetic polynucleotide”

SEQ ID NO: 38 - protein, 35 aa, Artificial Sequence
  source: /note=“Description of Artificial Sequence: Synthetic polypeptide”
TKKKYSSSVHDPNGEYMFMRAVNTAKKSRLTDVTL

SEQ ID NO: 39 - DNA, 105 bp, Artificial Sequence
  source: /note=“Description of Artificial Sequence: Synthetic polynucleotide”
acaaaaaagaagtattcatccagtgtgcacgaccctaacggtgaatacatgttcatgagagcagtgaacacagccaaaaaatccagactcacagatgtgacccta

SEQ ID NO: 40 - DNA, 30 bp, Artificial Sequence
  source: /note=“Description of Artificial Sequence: Synthetic oligonucleotide”
ggtggcggaggttctggaggtgggggttcc

SEQ ID NO: 41 - protein, 4 aa, Artificial Sequence
  source: /note=“Description of Artificial Sequence: Synthetic peptide”
  source: /note=“See specification as filed for detailed description of substitutions and preferred embodiments”
GGGS

SEQ ID NO: 42 - protein, 40 aa, Artificial Sequence
  source: /note=“Description of Artificial Sequence: Synthetic polypeptide”
  SITE (1)..(40): /note=“This sequence may encompass 1-10 ′Gly Gly Gly Ser′ repeating units”
GGGSGGGSGGGSGGGSGGGSGGGSGGGSGGGSGGGSGGGS

SEQ ID NO: 43 - protein, 18 aa, Artificial Sequence
  source: /note=“Description of Artificial Sequence: Synthetic peptide”
GSTSGSGKPGSGEGSTKG

SEQ ID NO: 44 - protein, 5 aa, Artificial Sequence
  source: /note=“Description of Artificial Sequence: Synthetic peptide”
SYAMS

SEQ ID NO: 45 - protein, 17 aa, Artificial Sequence
  source: /note=“Description of Artificial Sequence: Synthetic peptide”
AISGSGGSTYYADSVKG

SEQ ID NO: 46 - protein, 14 aa, Artificial Sequence
  source: /note=“Description of Artificial Sequence: Synthetic peptide”
REWVPYDVSWYFDY

SEQ ID NO: 47 - protein, 7 aa, Artificial Sequence
  source: /note=“Description of Artificial Sequence: Synthetic peptide”
GFTFSSY

SEQ ID NO: 48 - protein, 6 aa, Artificial Sequence
  source: /note=“Description of Artificial Sequence: Synthetic peptide”
SGSGGS

SEQ ID NO: 49 - protein, 8 aa, Artificial Sequence
  source: /note=“Description of Artificial Sequence: Synthetic peptide”
GFTFSSYA

SEQ ID NO: 50 - protein, 8 aa, Artificial Sequence
  source: /note=“Description of Artificial Sequence: Synthetic peptide”
ISGSGGST

SEQ ID NO: 51 - protein, 16 aa, Artificial Sequence
  source: /note=“Description of Artificial Sequence: Synthetic peptide”
ARREWVPYDVSWYFDY

SEQ ID NO: 52 - protein, 123 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWVPYDVSWYFDYWGQGTLVTVSS

SEQ ID NO: 53 - DNA, 369 bp, Artificial Sequence
  source: /note=“Description of Artificial Sequence: Synthetic polynucleotide”
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtgggtgccctacgatgtcagctggtacttcgactactggggacagggcactctcgtgactgtgtcctcc

SEQ ID NO: 54 - protein, 11 aa, Artificial Sequence
  source: /note=“Description of Artificial Sequence: Synthetic peptide”
RASQSISSYLN

SEQ ID NO: 55 - protein, 7 aa, Artificial Sequence
  source: /note=“Description of Artificial Sequence: Synthetic peptide”
AASSLQS

SEQ ID NO: 56 - protein, 9 aa, Artificial Sequence
  source: /note=“Description of Artificial Sequence: Synthetic peptide”
QQSYSTPLT

SEQ ID NO: 57 - protein, 7 aa, Artificial Sequence
  source: /note=“Description of Artificial Sequence: Synthetic peptide”
SQSISSY

SEQ ID NO: 58 - protein, 3 aa, Artificial Sequence
  source: /note=“Description of Artificial Sequence: Synthetic peptide”
AAS

SEQ ID NO: 59 - protein, 6 aa, Artificial Sequence
  source: /note=“Description of Artificial Sequence: Synthetic peptide”
SYSTPL

SEQ ID NO: 60 - protein, 6 aa, Artificial Sequence
  source: /note=“Description of Artificial Sequence: Synthetic peptide”
QSISSY

SEQ ID NO: 61 - protein, 107 aa, Artificial Sequence [sequence omitted]

SEQ ID NO: 62 - DNA, 321 bp, Artificial Sequence
  source: /note=“Description of Artificial Sequence: Synthetic polynucleotide”
gacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaag

SEQ ID NO: 63 - protein, 20 aa, Artificial Sequence
  source: /note=“Description of Artificial Sequence: Synthetic peptide”
GGGGSGGGGSGGGGSGGGGS

SEQ ID NO: 64 - protein, 250 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWVPYDVSWYFDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIK

SEQ ID NO: 65 - DNA, 750 bp, Artificial Sequence
  source: /note=“Description of Artificial Sequence: Synthetic polynucleotide”
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtgggtgccctacgatgtcagctggtacttcgactactggggacagggcactctcgtgactgtgtcctccggtggtggtggatcggggggtggtggttcgggcggaggaggatctggaggaggagggtcggacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaag

SEQ ID NO: 66 - protein, 473 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWVPYDVSWYFDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - DNA, 1419 bp, Artificial Sequence
  source: /note=“Description of Artificial Sequence: Synthetic polynucleotide”
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtgggtgccctacgatgtcagctggtacttcgactactggggacagggcactctcgtgactgtgtcctccggtggtggtggatcggggggtggtggttcgggcggaggaggatctggaggaggagggtcggacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 68 - protein, 12 aa, Artificial Sequence
  source: /note=“Description of Artificial Sequence: Synthetic peptide”
REWWYDDWYLDY

SEQ ID NO: 69 - protein, 14 aa, Artificial Sequence
  source: /note=“Description of Artificial Sequence: Synthetic peptide”
ARREWWYDDWYLDY

SEQ ID NO: 70 - protein, 121 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWWYDDWYLDYWGQGTLVTVSS

SEQ ID NO: 71 - DNA, 363 bp, Artificial Sequence
  source: /note=“Description of Artificial Sequence: Synthetic polynucleotide”
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtggtggtacgacgattggtacctggactactggggacagggcactctcgtgactgtgtcctcc

SEQ ID NO: 72 - protein, 248 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWWYDDWYLDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIK

SEQ ID NO: 73 - DNA, 744 bp, Artificial Sequence
  source: /note=“Description of Artificial Sequence: Synthetic polynucleotide”
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtggtggtacgacgattggtacctggactactggggacagggcactctcgtgactgtgtcctccggtggtggtggatcggggggtggtggttcgggcggaggaggatctggaggaggagggtcggacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaag

SEQ ID NO: 74 - protein, 471 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWWYDDWYLDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - DNA, 1413 bp, Artificial Sequence
  source: /note=“Description of Artificial Sequence: Synthetic polynucleotide”
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtggtggtacgacgattggtacctggactactggggacagggcactctcgtgactgtgtcctccggtggtggtggatcggggggtggtggttcgggcggaggaggatctggaggaggagggtcggacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 76 - protein, 12 aa, Artificial Sequence
  source: /note=“Description of Artificial Sequence: Synthetic peptide”
REWWGESWLFDY

SEQ ID NO: 77 - protein, 14 aa, Artificial Sequence
  source: /note=“Description of Artificial Sequence: Synthetic peptide”
ARREWWGESWLFDY

SEQ ID NO: 78 - protein, 121 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWWGESWLFDYWGQGTLVTVSS

SEQ ID NO: 79 - DNA, 363 bp, Artificial Sequence
  source: /note=“Description of Artificial Sequence: Synthetic polynucleotide”
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtggtggggagaaagctggctgttcgactactggggacagggcactctcgtgactgtgtcctcc

SEQ ID NO: 80 - protein, 248 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWWGESWLFDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIK

SEQ ID NO: 81 - DNA, 744 bp, Artificial Sequence
  source: /note=“Description of Artificial Sequence: Synthetic polynucleotide”
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtggtggggagaaagctggctgttcgactactggggacagggcactctcgtgactgtgtcctccggtggtggtggatcggggggtggtggttcgggcggaggaggatctggaggaggagggtcggacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaag

SEQ ID NO: 82 - protein, 471 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWWGESWLFDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 83 - DNA, 1413 bp, Artificial Sequence
  source: /note=“Description of Artificial Sequence: Synthetic polynucleotide”
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtggtggggagaaagctggctgttcgactactggggacagggcactctcgtgactgtgtcctccggtggtggtggatcggggggtggtggttcgggcggaggaggatctggaggaggagggtcggacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 84 - protein, 14 aa, Artificial Sequence
  source: /note=“Description of Artificial Sequence: Synthetic peptide”
  VARIANT (4)..(4): /replace=“ ”
  VARIANT (5)..(5): /replace=“ ”
  VARIANT (6)..(6): /replace=“Tyr”
  VARIANT (7)..(7): /replace=“Tyr” or “Asp”
  VARIANT (8)..(8): /replace=“Asp” or “Val”
  VARIANT (9)..(9): /replace=“Asp”
  VARIANT (11)..(11): /replace=“Tyr”
  VARIANT (12)..(12): /replace=“Leu”
  SITE (1)..(14): /note=“Variant residues given in the sequence have no preference with respect to those in the annotations for variant positions”
REWVPWGESWLFDY

SEQ ID NO: 85 - protein, 16 aa, Artificial Sequence
  source: /note=“Description of Artificial Sequence: Synthetic peptide”
  VARIANT (6)..(6): /replace=“ ”
  VARIANT (7)..(7): /replace=“ ”
  VARIANT (8)..(8): /replace=“Tyr”
  VARIANT (9)..(9): /replace=“Tyr” or “Asp”
  VARIANT (10)..(10): /replace=“Asp” or “Val”
  VARIANT (11)..(11): /replace=“Asp”
  VARIANT (13)..(13): /replace=“Tyr”
  VARIANT (14)..(14): /replace=“Leu”
  SITE (1)..(16): /note=“Variant residues given in the sequence have no preference with respect to those in the annotations for variant positions”
ARREWVPWGESWLFDY

SEQ ID NO: 86 - protein, 5 aa, Artificial Sequence
  source: /note=“Description of Artificial Sequence: Synthetic peptide”
SYGMH

SEQ ID NO: 87 - protein, 17 aa, Artificial Sequence
  source: /note=“Description of Artificial Sequence: Synthetic peptide”
VISYDGSNKYYADSVKG

SEQ ID NO: 88 - protein, 14 aa, Artificial Sequence
  source: /note=“Description of Artificial Sequence: Synthetic peptide”
SGYALHDDYYGLDV

SEQ ID NO: 89 - protein, 6 aa, Artificial Sequence
  source: /note=“Description of Artificial Sequence: Synthetic peptide”
SYDGSN

SEQ ID NO: 90 - protein, 8 aa, Artificial Sequence
  source: /note=“Description of Artificial Sequence: Synthetic peptide”
GFTFSSYG

SEQ ID NO: 91 - protein, 8 aa, Artificial Sequence
  source: /note=“Description of Artificial Sequence: Synthetic peptide”
ISYDGSNK

SEQ ID NO: 92 - protein, 16 aa, Artificial Sequence
  source: /note=“Description of Artificial Sequence: Synthetic peptide”
GGSGYALHDDYYGLDV

SEQ ID NO: 93 - protein, 123 aa, Artificial Sequence
  source: /note=“Description of Artificial Sequence: Synthetic polypeptide”
QVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCGGSGYALHDDYYGLDVWGQGTLVTVSS

SEQ ID NO: 94 - DNA, 369 bp, Artificial Sequence
  source: /note=“Description of Artificial Sequence: Synthetic polynucleotide”
caagtgcagctgcaggaatccggtggcggagtcgtgcagcctggaaggagcctgagactctcatgcgccgcgtcagggttcaccttttcctcctacgggatgcattgggtcagacaggcccccggaaagggactcgaatgggtggctgtgatcagctacgacggctccaacaagtactacgccgactccgtgaaaggccggttcactatctcccgggacaactccaagaacacgctgtatctgcaaatgaattcactgcgcgcggaggataccgctgtgtactactgcggtggctccggttacgccctgcacgatgactattacggccttgacgtctggggccagggaaccctcgtgactgtgtccagc

SEQ ID NO: 95 - protein, 14 aa, Artificial Sequence
  source: /note=“Description of Artificial Sequence: Synthetic peptide”
TGTSSDVGGYNYVS

SEQ ID NO: 96 - protein, 7 aa, Artificial Sequence
  source: /note=“Description of Artificial Sequence: Synthetic peptide”
DVSNRPS

SEQ ID NO: 97 - protein, 11 aa, Artificial Sequence
  source: /note=“Description of Artificial Sequence: Synthetic peptide”
SSYTSSSTLYV

SEQ ID NO: 98 - protein, 10 aa, Artificial Sequence
  source: /note=“Description of Artificial Sequence: Synthetic peptide”
TSSDVGGYNY

SEQ ID NO: 99 - protein, 3 aa, Artificial Sequence
  source: /note=“Description of Artificial Sequence: Synthetic peptide”
DVS

SEQ ID NO: 100 - protein, 8 aa, Artificial Sequence
  source: /note=“Description of Artificial Sequence: Synthetic peptide”
YTSSSTLY

SEQ ID NO: 101 - protein, 9 aa, Artificial Sequence
  source: /note=“Description of Artificial Sequence: Synthetic peptide”
SSDVGGYNY

SEQ ID NO: 102 - protein, 111 aa, Artificial Sequence
  source: /note=“Description of Artificial Sequence: Synthetic polypeptide”
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGSGTKVTVL

SEQ ID NO: 103 - DNA, 333 bp, Artificial Sequence
  source: /note=“Description of Artificial Sequence: Synthetic polynucleotide”
cagagcgcactgactcagccggcatccgtgtccggtagccccggacagtcgattaccatctcctgtaccggcacctcctccgacgtgggagggtacaactacgtgtcgtggtaccagcagcacccaggaaaggcccctaagttgatgatctacgatgtgtcaaaccgcccgtctggagtctccaaccggttctccggctccaagtccggcaacaccgccagcctgaccattagcgggctgcaagccgaggatgaggccgactactactgctcgagctacacatcctcgagcaccctctacgtgttcggctcggggactaaggtcaccgtgctg

SEQ ID NO: 104 - protein, 15 aa, Artificial Sequence
  source: /note=“Description of Artificial Sequence: Synthetic peptide”
GGGGSGGGGSGGGGS

SEQ ID NO: 105 - protein, 249 aa, Artificial Sequence
  source: /note=“Description of Artificial Sequence: Synthetic polypeptide”
QVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCGGSGYALHDDYYGLDVWGQGTLVTVSSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGSGTKVTVL

SEQ ID NO: 106 - DNA, 747 bp, Artificial Sequence
  source: /note=“Description of Artificial Sequence: Synthetic polynucleotide”
caagtgcagctgcaggaatccggtggcggagtcgtgcagcctggaaggagcctgagactctcatgcgccgcgtcagggttcaccttttcctcctacgggatgcattgggtcagacaggcccccggaaagggactcgaatgggtggctgtgatcagctacgacggctccaacaagtactacgccgactccgtgaaaggccggttcactatctcccgggacaactccaagaacacgctgtatctgcaaatgaattcactgcgcgcggaggataccgctgtgtactactgcggtggctccggttacgccctgcacgatgactattacggccttgacgtctggggccagggaaccctcgtgactgtgtccagcggtggaggaggttcgggcggaggaggatcaggagggggtggatcgcagagcgcactgactcagccggcatccgtgtccggtagccccggacagtcgattaccatctcctgtaccggcacctcctccgacgtgggagggtacaactacgtgtcgtggtaccagcagcacccaggaaaggcccctaagttgatgatctacgatgtgtcaaaccgcccgtctggagtctccaaccggttctccggctccaagtccggcaacaccgccagcctgaccattagcgggctgcaagccgaggatgaggccgactactactgctcgagctacacatcctcgagcaccctctacgtgttcggctcggggactaaggtcaccgtgctg

SEQ ID NO: 107 - protein, 472 aa, Artificial Sequence
  source: /note=“Description of Artificial Sequence: Synthetic polypeptide”
QVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCGGSGYALHDDYYGLDVWGQGTLVTVSSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGSGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 108 - DNA, 1416 bp, Artificial Sequence
  source: /note=“Description of Artificial Sequence: Synthetic polynucleotide”
caagtgcagctgcaggaatccggtggcggagtcgtgcagcctggaaggagcctgagactctcatgcgccgcgtcagggttcaccttttcctcctacgggatgcattgggtcagacaggcccccggaaagggactcgaatgggtggctgtgatcagctacgacggctccaacaagtactacgccgactccgtgaaaggccggttcactatctcccgggacaactccaagaacacgctgtatctgcaaatgaattcactgcgcgcggaggataccgctgtgtactactgcggtggctccggttacgccctgcacgatgactattacggccttgacgtctggggccagggaaccctcgtgactgtgtccagcggtggaggaggttcgggcggaggaggatcaggagggggtggatcgcagagcgcactgactcagccggcatccgtgtccggtagccccggacagtcgattaccatctcctgtaccggcacctcctccgacgtgggagggtacaactacgtgtcgtggtaccagcagcacccaggaaaggcccctaagttgatgatctacgatgtgtcaaaccgcccgtctggagtctccaaccggttctccggctccaagtccggcaacaccgccagcctgaccattagcgggctgcaagccgaggatgaggccgactactactgctcgagctacacatcctcgagcaccctctacgtgttcggctcggggactaaggtcaccgtgctgaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 109 - protein, 17 aa, Artificial Sequence
  source: /note=“Description of Artificial Sequence: Synthetic peptide”
VISYKGSNKYYADSVKG

SEQ ID NO: 110 - protein, 6 aa, Artificial Sequence
  source: /note=“Description of Artificial Sequence: Synthetic peptide”
SYKGSN

SEQ ID NO: 111 - protein, 8 aa, Artificial Sequence
  source: /note=“Description of Artificial Sequence: Synthetic peptide”
ISYKGSNK

SEQ ID NO: 112 - protein, 123 aa, Artificial Sequence
  source: /note=“Description of Artificial Sequence: Synthetic polypeptide”
QVQLVESGGGVVQPGRSLRLSCAASGFTFSSYGMHWVRQAPGKGLEWVAVISYKGSNKYYADSVKGRFTISRDNSKNTLYLQMNSLRAEDTAVYYCGGSGYALHDDYYGLDVWGQGTLVTVSS

SEQ ID NO: 113 - DNA, 369 bp, Artificial Sequence
  source: /note=“Description of Artificial Sequence: Synthetic polynucleotide”
caagtgcagcttgtcgaatcgggaggcggagtggtgcagcctggacgatcgctccggctctcatgtgccgcgagcggattcaccttctcgagctacggcatgcactgggtcagacaagccccaggaaagggcctggaatgggtggctgtcatctcgtacaagggctcaaacaagtactacgccgactccgtgaagggccggttcaccatctcccgcgataactccaagaataccctctatctgcaaatgaacagcctgagggccgaggatactgcagtgtactactgcgggggttcaggctacgcgctgcacgacgactactacggattggacgtctggggccaaggaactcttgtgaccgtgtcctct

SEQ ID NO: 114 - protein, 7 aa, Artificial Sequence
  source: /note=“Description of Artificial Sequence: Synthetic peptide”
EVSNRLR

SEQ ID NO: 115 - protein, 11 aa, Artificial Sequence
  source: /note=“Description of Artificial Sequence: Synthetic peptide”
SSYTSSSALYV

SEQ ID NO: 116 - protein, 3 aa, Artificial Sequence
  source: /note=“Description of Artificial Sequence: Synthetic peptide”
EVS

SEQ ID NO: 117 - protein, 8 aa, Artificial Sequence
  source: /note=“Description of Artificial Sequence: Synthetic peptide”
YTSSSALY

SEQ ID NO: 118 - protein, 111 aa, Artificial Sequence
  source: /note=“Description of Artificial Sequence: Synthetic polypeptide”
QSALTQPASVSGSPGQSITISCTGTSSDVGGYNYVSWYQQHPGKAPKLMIYEVSNRLRGVSNRFSGSKSGNTASLTISGLQAEDEADYYCSSYTSSSALYVFGSGTKVTVL

SEQ ID NO: 119 - DNA, 333 bp, Artificial Sequence
  source: /note=“Description of Artificial Sequence: Synthetic polynucleotide”
cagagcgcgctgactcagcctgcctccgtgagcggttcgccgggacagtccattaccatttcgtgcaccgggacctcctccgacgtgggaggctacaactacgtgtcctggtaccagcagcatcccggaaaggccccgaagctgatgatctacgaagtgtcgaacagactgcggggagtctccaaccgcttttccgggtccaagtccggcaacaccgccagcctgaccatcagcgggctccaggcagaagatgaggctgactattactgctcctcctacacgtcaagctccgccctctacgtgttcgggtccgggaccaaagtcactgtgctg

SEQ ID NO: 120 - protein, 254 aa, Artificial Sequence
  source: /note=“Description of Artificial Sequence: Synthetic polypeptide”
QVQLVESGGGVVQPGRSLRLSCAASGFTFSSYGMHWVRQAPGKGLEWVAVISYKGSNKYYADSVKGRFTISRDNSKNTLYLQMNSLRAEDTAVYYCGGSGYALHDDYYGLDVWGQGTLVTVSSGGGGSGGGGSGGGGSGGGGSQSALTQPASVSGSPGQSITISCTGTSSDVGGYNYVSWYQQHPGKAPKLMIYEVSNRLRGVSNRFSGSKSGNTASLTISGLQAEDEADYYCSSYTSSSALYVFGSGTKVTVL

SEQ ID NO: 121 - DNA, 762 bp, Artificial Sequence
  source: /note=“Description of Artificial Sequence: Synthetic polynucleotide”
caagtgcagcttgtcgaatcgggaggcggagtggtgcagcctggacgatcgctccggctctcatgtgccgcgagcggattcaccttctcgagctacggcatgcactgggtcagacaagccccaggaaagggcctggaatgggtggctgtcatctcgtacaagggctcaaacaagtactacgccgactccgtgaagggccggttcaccatctcccgcgataactccaagaataccctctatctgcaaatgaacagcctgagggccgaggatactgcagtgtactactgcgggggttcaggctacgcgctgcacgacgactactacggattggacgtctggggccaaggaactcttgtgaccgtgtcctctggtggaggcggatcagggggtggcggatctgggggtggtggttccgggggaggaggatcgcagagcgcgctgactcagcctgcctccgtgagcggttcgccgggacagtccattaccatttcgtgcaccgggacctcctccgacgtgggaggctacaactacgtgtcctggtaccagcagcatcccggaaaggccccgaagctgatgatctacgaagtgtcgaacagactgcggggagtctccaaccgcttttccgggtccaagtccggcaacaccgccagcctgaccatcagcgggctccaggcagaagatgaggctgactattactgctcctcctacacgtcaagctccgccctctacgtgttcgggtccgggaccaaagtcactgtgctg

SEQ ID NO: 122 - protein, 477 aa, Artificial Sequence
  source: /note=“Description of Artificial Sequence: Synthetic polypeptide”
QVQLVESGGGVVQPGRSLRLSCAASGFTFSSYGMHWVRQAPGKGLEWVAVISYKGSNKYYADSVKGRFTISRDNSKNTLYLQMNSLRAEDTAVYYCGGSGYALHDDYYGLDVWGQGTLVTVSSGGGGSGGGGSGGGGSGGGGSQSALTQPASVSGSPGQSITISCTGTSSDVGGYNYVSWYQQHPGKAPKLMIYEVSNRLRGVSNRFSGSKSGNTASLTISGLQAEDEADYYCSSYTSSSALYVFGSGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - DNA, 1431 bp, Artificial Sequence
  source: /note=“Description of Artificial Sequence: Synthetic polynucleotide”
caagtgcagcttgtcgaatcgggaggcggagtggtgcagcctggacgatcgctccggctctcatgtgccgcgagcggattcaccttctcgagctacggcatgcactgggtcagacaagccccaggaaagggcctggaatgggtggctgtcatctcgtacaagggctcaaacaagtactacgccgactccgtgaagggccggttcaccatctcccgcgataactccaagaataccctctatctgcaaatgaacagcctgagggccgaggatactgcagtgtactactgcgggggttcaggctacgcgctgcacgacgactactacggattggacgtctggggccaaggaactcttgtgaccgtgtcctctggtggaggcggatcagggggtggcggatctgggggtggtggttccgggggaggaggatcgcagagcgcgctgactcagcctgcctccgtgagcggttcgccgggacagtccattaccatttcgtgcaccgggacctcctccgacgtgggaggctacaactacgtgtcctggtaccagcagcatcccggaaaggccccgaagctgatgatctacgaagtgtcgaacagactgcggggagtctccaaccgcttttccgggtccaagtccggcaacaccgccagcctgaccatcagcgggctccaggcagaagatgaggctgactattactgctcctcctacacgtcaagctccgccctctacgtgttcgggtccgggaccaaagtcactgtgctgaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 124 - protein, 111 aa, Artificial Sequence
  source: /note=“Description of Artificial Sequence: Synthetic polypeptide”
QSALTQPASVSGSPGQSITISCTGTSSDVGGYNYVSWYQQHPGKAPKLMIYEVSNRLRGVSNRFSGSKSGNTASLTISGLQAEDEADYYCSSYTSSSTLYVFGSGTKVTVL

SEQ ID NO: 125 - DNA, 333 bp, Artificial Sequence
  source: /note=“Description of Artificial Sequence: Synthetic polynucleotide”
cagagcgcgctgactcagcctgcctccgtgagcggttcgccgggacagtccattaccatttcgtgcaccgggacctcctccgacgtgggaggctacaactacgtgtcctggtaccagcagcatcccggaaaggccccgaagctgatgatctacgaagtgtcgaacagactgcggggagtctccaaccgcttttccgggtccaagtccggcaacaccgccagcctgaccatcagcgggctccaggcagaagatgaggctgactattactgctcctcctacacgtcaagctccaccctctacgtgttcgggtccgggaccaaagtcactgtgctg

SEQ ID NO: 126 - protein, 254 aa, Artificial Sequence
  source: /note=“Description of Artificial Sequence: Synthetic polypeptide”
QVQLVESGGGVVQPGRSLRLSCAASGFTFSSYGMHWVRQAPGKGLEWVAVISYKGSNKYYADSVKGRFTISRDNSKNTLYLQMNSLRAEDTAVYYCGGSGYALHDDYYGLDVWGQGTLVTVSSGGGGSGGGGSGGGGSGGGGSQSALTQPASVSGSPGQSITISCTGTSSDVGGYNYVSWYQQHPGKAPKLMIYEVSNRLRGVSNRFSGSKSGNTASLTISGLQAEDEADYYCSSYTSSSTLYVFGSGTKVTVL

SEQ ID NO: 127 - DNA, 762 bp, Artificial Sequence
  source: /note=“Description of Artificial Sequence: Synthetic polynucleotide”
caagtgcagcttgtcgaatcgggaggcggagtggtgcagcctggacgatcgctccggctctcatgtgccgcgagcggattcaccttctcgagctacggcatgcactgggtcagacaagccccaggaaagggcctggaatgggtggctgtcatctcgtacaagggctcaaacaagtactacgccgactccgtgaagggccggttcaccatctcccgcgataactccaagaataccctctatctgcaaatgaacagcctgagggccgaggatactgcagtgtactactgcgggggttcaggctacgcgctgcacgacgactactacggattggacgtctggggccaaggaactcttgtgaccgtgtcctctggtggaggcggatcagggggtggcggatctgggggtggtggttccgggggaggaggatcgcagagcgcgctgactcagcctgcctccgtgagcggttcgccgggacagtccattaccatttcgtgcaccgggacctcctccgacgtgggaggctacaactacgtgtcctggtaccagcagcatcccggaaaggccccgaagctgatgatctacgaagtgtcgaacagactgcggggagtctccaaccgcttttccgggtccaagtccggcaacaccgccagcctgaccatcagcgggctccaggcagaagatgaggctgactattactgctcctcctacacgtcaagctccaccctctacgtgttcgggtccgggaccaaagtcactgtgctg

SEQ ID NO: 128 - protein, 477 aa, Artificial Sequence
  source: /note=“Description of Artificial Sequence: Synthetic polypeptide”
QVQLVESGGGVVQPGRSLRLSCAASGFTFSSYGMHWVRQAPGKGLEWVAVISYKGSNKYYADSVKGRFTISRDNSKNTLYLQMNSLRAEDTAVYYCGGSGYALHDDYYGLDVWGQGTLVTVSSGGGGSGGGGSGGGGSGGGGSQSALTQPASVSGSPGQSITISCTGTSSDVGGYNYVSWYQQHPGKAPKLMIYEVSNRLRGVSNRFSGSKSGNTASLTISGLQAEDEADYYCSSYTSSSTLYVFGSGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - DNA, 1431 bp, Artificial Sequence
  source: /note=“Description of Artificial Sequence: Synthetic polynucleotide”
caagtgcagcttgtcgaatcgggaggcggagtggtgcagcctggacgatcgctccggctctcatgtgccgcgagcggattcaccttctcgagctacggcatgcactgggtcagacaagccccaggaaagggcctggaatgggtggctgtcatctcgtacaagggctcaaacaagtactacgccgactccgtgaagggccggttcaccatctcccgcgataactccaagaataccctctatctgcaaatgaacagcctgagggccgaggatactgcagtgtactactgcgggggttcaggctacgcgctgcacgacgactactacggattggacgtctggggccaaggaactcttgtgaccgtgtcctctggtggaggcggatcagggggtggcggatctgggggtggtggttccgggggaggaggatcgcagagcgcgctgactcagcctgcctccgtgagcggttcgccgggacagtccattaccatttcgtgcaccgggacctcctccgacgtgggaggctacaactacgtgtcctggtaccagcagcatcccggaaaggccccgaagctgatgatctacgaagtgtcgaacagactgcggggagtctccaaccgcttttccgggtccaagtccggcaacaccgccagcctgaccatcagcgggctccaggcagaagatgaggctgactattactgctcctcctacacgtcaagctccaccctctacgtgttcgggtccgggaccaaagtcactgtgctgaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 130 - protein, 17 aa, Artificial Sequence
  source: /note=“Description of Artificial Sequence: Synthetic peptide”
  VARIANT (5)..(5): /replace=“Lys”
  SITE (1)..(17): /note=“Variant residues given in the sequence have no preference with respect to those in the annotations for variant positions”
VISYDGSNKYYADSVKG

SEQ ID NO: 131 - protein, 7 aa, Artificial Sequence
  source: /note=“Description of Artificial Sequence: Synthetic peptide”
  VARIANT (1)..(1): /replace=“Glu”
  VARIANT (6)..(6): /replace=“Leu”
  VARIANT (7)..(7): /replace=“Arg”
  SITE (1)..(7): /note=“Variant residues given in the sequence have no preference with respect to those in the annotations for variant positions”
DVSNRPS

SEQ ID NO: 132 - protein, 11 aa, Artificial Sequence
  source: /note=“Description of Artificial Sequence: Synthetic peptide”
  VARIANT (8)..(8): /replace=“Ala”
  SITE (1)..(11): /note=“Variant residues given in the sequence have no preference with respect to those in the annotations for variant positions”
SSYTSSSTLYV

SEQ ID NO: 133 - protein, 6 aa, Artificial Sequence
  source: /note=“Description of Artificial Sequence: Synthetic peptide”
  VARIANT (3)..(3): /replace=“Lys”
  SITE (1)..(6): /note=“Variant residues given in the sequence have no preference with respect to those in the annotations for variant positions”
SYDGSN

SEQ ID NO: 134 - protein, 3 aa, Artificial Sequence
  source: /note=“Description of Artificial Sequence: Synthetic peptide”
  VARIANT (1)..(1): /replace=“Glu”
  SITE (1)..(3): /note=“Variant residues given in the sequence have no preference with respect to those in the annotations for variant positions”
DVS

SEQ ID NO: 135 - protein, 8 aa, Artificial Sequence
  source: /note=“Description of Artificial Sequence: Synthetic peptide”
  VARIANT (6)..(6): /replace=“Ala”
  SITE (1)..(8): /note=“Variant residues given in the sequence have no preference with respect to those in the annotations for variant positions”
YTSSSTLY

SEQ ID NO: 136 - protein, 8 aa, Artificial Sequence
  source: /note=“Description of Artificial Sequence: Synthetic peptide”
  VARIANT (4)..(4): /replace=“Lys”
  SITE (1)..(8): /note=“Variant residues given in the sequence have no preference with respect to those in the annotations for variant positions”
ISYDGSNK

SEQ ID NO: 137 - protein, 5 aa, Artificial Sequence
  source: /note=“Description of Artificial Sequence: Synthetic peptide”
GFWMS

SEQ ID NO: 138 - protein, 17 aa, Artificial Sequence
  source: /note=“Description of Artificial Sequence: Synthetic peptide”
NIKQDGSEKYYVDSVRG

SEQ ID NO: 139 - protein, 9 aa, Artificial Sequence
  source: /note=“Description of Artificial Sequence: Synthetic peptide”
ALDYYGMDV

SEQ ID NO: 140 - protein, 7 aa, Artificial Sequence
  source: /note=“Description of Artificial Sequence: Synthetic peptide”
GFTFSGF

SEQ ID NO: 141 - protein, 6 aa, Artificial Sequence
  source: /note=“Description of Artificial Sequence: Synthetic peptide”
KQDGSE

SEQ ID NO: 142 - protein, 8 aa, Artificial Sequence
  source: /note=“Description of Artificial Sequence: Synthetic peptide”
GFTFSGFW

SEQ ID NO: 143 - protein, 8 aa, Artificial Sequence
  source: /note=“Description of Artificial Sequence: Synthetic peptide”
IKQDGSEK

SEQ ID NO: 144 - protein, 11 aa, Artificial Sequence
  source: /note=“Description of Artificial Sequence: Synthetic peptide”
ARALDYYGMDV

SEQ ID NO: 145 - protein, 118 aa, Artificial Sequence
  source: /note=“Description of Artificial Sequence: Synthetic polypeptide”
EVQLVESGGGLVQPGGSLRLSCAASGFTFSGFWMSWVRQAPGKGLEWVANIKQDGSEKYYVDSVRGRFTISRDNAKNSLYLQMNSLRAEDTAVYYCARALDYYGMDVWGQGTTVTVSS

SEQ ID NO: 146 - DNA, 354 bp, Artificial Sequence
  source: /note=“Description of Artificial Sequence: Synthetic polynucleotide”
gaagtgcaactggtggagagcggtggagggcttgtccagcccggaggatcgctgcggctgtcctgtgctgcgtccgggttcaccttctccggcttctggatgtcctgggtcagacaggcaccgggaaagggcctcgaatgggtggccaacatcaagcaggatggctccgagaagtactacgtcgactccgtgagaggccgcttcaccatctcccgggacaacgccaagaactcgctgtacctccaaatgaatagcctcagggcggaagatactgctgtgtattactgcgcacgcgcccttgactactacggcatggacgtctggggccaagggaccactgtgaccgtgtctagc

SEQ ID NO: 147 - protein, 17 aa, Artificial Sequence
  source: /note=“Description of Artificial Sequence: Synthetic peptide”
RSSQSLLDSDDGNTYLD

SEQ ID NO: 148 - protein, 7 aa, Artificial Sequence
  source: /note=“Description of Artificial Sequence: Synthetic peptide”
TLSYRAS

SEQ ID NO: 149 - protein, 10 aa, Artificial Sequence
  source: /note=“Description of Artificial Sequence: Synthetic peptide”
TQRLEFPSIT

SEQ ID NO: 150 - protein, 13 aa, Artificial Sequence
  source: /note=“Description of Artificial Sequence: Synthetic peptide”
SQSLLDSDDGNTY

SEQ ID NO: 151 - protein, 3 aa, Artificial Sequence
  source: /note=“Description of Artificial Sequence: Synthetic peptide”
TLS

SEQ ID NO: 152 - protein, 7 aa, Artificial Sequence
  source: /note=“Description of Artificial Sequence: Synthetic peptide”
RLEFPSI

SEQ ID NO: 153 - protein, 12 aa, Artificial Sequence
  source: /note=“Description of Artificial Sequence: Synthetic peptide”
QSLLDSDDGNTY

SEQ ID NO: 154 - protein, 114 aa, Artificial Sequence
  source: /note=“Description of Artificial Sequence: Synthetic polypeptide”
DIVMTQTPLSLPVTPGEPASISCRSSQSLLDSDDGNTYLDWYLQKPGQSPRLLIYTLSYRASGVPDRFSGSGSGTDFTLKISRVEAEDVGLYYCTQRLEFPSITFGQGTRLEIK

SEQ ID NO: 155 - DNA, 342 bp, Artificial Sequence
  source: /note=“Description of Artificial Sequence: Synthetic polynucleotide”
gatatcgtgatgacccagactcccctgtccctgcctgtgactcccggagaaccagcctccatttcctgccggtcctcccagtccctgctggacagcgacgacggcaacacttacctggactggtacttgcagaagccgggccaatcgcctcgcctgctgatctataccctgtcataccgggcctcaggagtgcctgaccgcttctcgggatcagggagcgggaccgatttcaccctgaaaatttcccgagtggaagccgaggacgtcggactgtactactgcacccagcgcctcgaattcccgtcgattacgtttggacagggtacccggcttgagatcaag

SEQ ID NO: 156 - protein, 252 aa, Artificial Sequence
  source: /note=“Description of Artificial Sequence: Synthetic polypeptide”
EVQLVESGGGLVQPGGSLRLSCAASGFTFSGFWMSWVRQAPGKGLEWVANIKQDGSEKYYVDSVRGRFTISRDNAKNSLYLQMNSLRAEDTAVYYCARALDYYGMDVWGQGTTVTVSSGGGGSGGGGSGGGGSGGGGSDIVMTQTPLSLPVTPGEPASISCRSSQSLLDSDDGNTYLDWYLQKPGQSPRLLIYTLSYRASGVPDRFSGSGSGTDFTLKISRVEAEDVGLYYCTQRLEFPSITFGQGTRLEIK

SEQ ID NO: 157 - DNA, 756 bp, Artificial Sequence
  source: /note=“Description of Artificial Sequence: Synthetic polynucleotide”
gaagtgcaactggtggagagcggtggagggcttgtccagcccggaggatcgctgcggctgtcctgtgctgcgtccgggttcaccttctccggcttctggatgtcctgggtcagacaggcaccgggaaagggcctcgaatgggtggccaacatcaagcaggatggctccgagaagtactacgtcgactccgtgagaggccgcttcaccatctcccgggacaacgccaagaactcgctgtacctccaaatgaatagcctcagggcggaagatactgctgtgtattactgcgcacgcgcccttgactactacggcatggacgtctggggccaagggaccactgtgaccgtgtctagcggaggcggaggttcagggggcggtggatcaggcggaggaggatcggggggtggtggatcggatatcgtgatgacccagactcccctgtccctgcctgtgactcccggagaaccagcctccatttcctgccggtcctcccagtccctgctggacagcgacgacggcaacacttacctggactggtacttgcagaagccgggccaatcgcctcgcctgctgatctataccctgtcataccgggcctcaggagtgcctgaccgcttctcgggatcagggagcgggaccgatttcaccctgaaaatttcccgagtggaagccgaggacgtcggactgtactactgcacccagcgcctcgaattcccgtcgattacgtttggacagggtacccggcttgagatcaag

SEQ ID NO: 158 - protein, 475 aa, Artificial Sequence
  source: /note=“Description of Artificial Sequence: Synthetic polypeptide”
EVQLVESGGGLVQPGGSLRLSCAASGFTFSGFWMSWVRQAPGKGLEWVANIKQDGSEKYYVDSVRGRFTISRDNAKNSLYLQMNSLRAEDTAVYYCARALDYYGMDVWGQGTTVTVSSGGGGSGGGGSGGGGSGGGGSDIVMTQTPLSLPVTPGEPASISCRSSQSLLDSDDGNTYLDWYLQKPGQSPRLLIYTLSYRASGVPDRFSGSGSGTDFTLKISRVEAEDVGLYYCTQRLEFPSITFGQGTRLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 159 - DNA, 1425 bp, Artificial Sequence
  source: /note=“Description of Artificial Sequence: Synthetic polynucleotide”
gaagtgcaactggtggagagcggtggagggcttgtccagcccggaggatcgctgcggctgtcctgtgctgcgtccgggttcaccttctccggcttctggatgtcctgggtcagacaggcaccgggaaagggcctcgaatgggtggccaacatcaagcaggatggctccgagaagtactacgtcgactccgtgagaggccgcttcaccatctcccgggacaacgccaagaactcgctgtacctccaaatgaatagcctcagggcggaagatactgctgtgtattactgcgcacgcgcccttgactactacggcatggacgtctggggccaagggaccactgtgaccgtgtctagcggaggcggaggttcagggggcggtggatcaggcggaggaggatcggggggtggtggatcggatatcgtgatgacccagactcccctgtccctgcctgtgactcccggagaaccagcctccatttcctgccggtcctcccagtccctgctggacagcgacgacggcaacacttacctggactggtacttgcagaagccgggccaatcgcctcgcctgctgatctataccctgtcataccgggcctcaggagtgcctgaccgcttctcgggatcagggagcgggaccgatttcaccctgaaaatttcccgagtggaagccgaggacgtcggactgtactactgcacccagcgcctcgaattcccgtcgattacgtttggacagggtacccggcttgagatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 160 - protein, 5 aa, Artificial Sequence
  source: /note=“Description of Artificial Sequence: Synthetic peptide”
SFRMN

SEQ ID NO: 161 - protein, 17 aa, Artificial Sequence
  source: /note=“Description of Artificial Sequence: Synthetic peptide”
SISSSSSYIYYADSVKG

SEQ ID NO: 162 - protein, 9 aa, Artificial Sequence
  source: /note=“Description of Artificial Sequence: Synthetic peptide”
WLSYYGMDV

SEQ ID NO: 163 - protein, 7 aa, Artificial Sequence
  source: /note=“Description of Artificial Sequence: Synthetic peptide”
GFTFSSF

SEQ ID NO: 164 - protein, 6 aa, Artificial Sequence
  source: /note=“Description of Artificial Sequence: Synthetic peptide”
SSSSSY

SEQ ID NO: 165 - protein, 8 aa, Artificial Sequence
  source: /note=“Description of Artificial Sequence: Synthetic peptide”
GFTFSSFR

SEQ ID NO: 166 - protein, 8 aa, Artificial Sequence
  source: /note=“Description of Artificial Sequence: Synthetic peptide”
ISSSSSYI

SEQ ID NO: 167 - protein, 11 aa, Artificial Sequence
  source: /note=“Description of Artificial Sequence: Synthetic peptide”
ARWLSYYGMDV

SEQ ID NO: 168 - protein, 118 aa, Artificial Sequence
  source: /note=“Description of Artificial Sequence: Synthetic polypeptide”
EVQLVESGGGLVKPGGSLRLSCAASGFTFSSFRMNWVRQAPGKGLEWVSSISSSSSYIYYADSVKGRFTISRDNAKNSLYLQMNSLRAEDTAVYYCARWLSYYGMDVWGQGTTVTVSS

SEQ ID NO: 169 - DNA, 354 bp, Artificial Sequence
  source: /note=“Description of Artificial Sequence: Synthetic polynucleotide”
gaagtgcaactggtggagagcggtggagggcttgtcaagcccggaggatcgctgcggctgtcctgtgctgcgtccgggttcaccttctcctcgttccgcatgaactgggtcagacaggcaccgggaaagggcctcgaatgggtgtcctcaatctcatcgtcctcgtcctacatctactacgccgactccgtgaaaggccgcttcaccatctcccgggacaacgccaagaactcgctgtacctccaaatgaatagcctcagggcggaagatactgctgtgtattactgcgcacgctggctttcctactacggcatggacgtctggggccaagggaccactgtgaccgtgtctagc

SEQ ID NO: 170 - protein, 7 aa, Artificial Sequence
  source: /note=“Description of Artificial Sequence: Synthetic peptide”
TLSFRAS

SEQ ID NO: 171 - protein, 9 aa, Artificial Sequence
  source: /note=“Description of Artificial Sequence: Synthetic peptide”
MQRIGFPIT

SEQ ID NO: 172 - protein, 6 aa, Artificial Sequence
  source: /note=“Description of Artificial Sequence: Synthetic peptide”
RIGFPI

SEQ ID NO: 173 - protein, 113 aa, Artificial Sequence
  source: /note=“Description of Artificial Sequence: Synthetic polypeptide”
DIVMTQTPLSLPVTPGEPASISCRSSQSLLDSDDGNTYLDWYLQKPGQSPQLLIYTLSFRASGVPDRFSGSGSGTDFTLKIRRVEAEDVGVYYCMQRIGFPITFGQGTRLEIK

SEQ ID NO: 174 - DNA, 339 bp, Artificial Sequence
  source: /note=“Description of Artificial Sequence: Synthetic polynucleotide”
gatatcgtgatgacccagactcccctgtccctgcctgtgactcccggagaaccagcctccatttcctgccggtcctcccagtccctgctggacagcgacgacggcaacacttacctggactggtacttgcagaagccgggccaatcgcctcagctgctgatctataccctgtcattccgggcctcaggagtgcctgaccgcttctcgggatcagggagcgggaccgatttcaccctgaaaattaggcgagtggaagccgaggacgtcggagtgtactactgcatgcagcgcatcggcttcccgattacgtttggacagggtacccggcttgagatcaag

SEQ ID NO: 175 - protein, 251 aa, Artificial Sequence
  source: /note=“Description of Artificial Sequence: Synthetic polypeptide”
EVQLVESGGGLVKPGGSLRLSCAASGFTFSSFRMNWVRQAPGKGLEWVSSISSSSSYIYYADSVKGRFTISRDNAKNSLYLQMNSLRAEDTAVYYCARWLSYYGMDVWGQGTTVTVSSGGGGSGGGGSGGGGSGGGGSDIVMTQTPLSLPVTPGEPASISCRSSQSLLDSDDGNTYLDWYLQKPGQSPQLLIYTLSFRASGVPDRFSGSGSGTDFTLKIRRVEAEDVGVYYCMQRIGFPITFGQGTRLEIK

SEQ ID NO: 176 - DNA, 753 bp, Artificial Sequence
  source: /note=“Description of Artificial Sequence: Synthetic polynucleotide”
gaagtgcaactggtggagagcggtggagggcttgtcaagcccggaggatcgctgcggctgtcctgtgctgcgtccgggttcaccttctcctcgttccgcatgaactgggtcagacaggcaccgggaaagggcctcgaatgggtgtcctcaatctcatcgtcctcgtcctacatctactacgccgactccgtgaaaggccgcttcaccatctcccgggacaacgccaagaactcgctgtacctccaaatgaatagcctcagggcggaagatactgctgtgtattactgcgcacgctggctttcctactacggcatggacgtctggggccaagggaccactgtgaccgtgtctagcggaggcggaggttcagggggcggtggatcaggcggaggaggatcggggggtggtggatcggatatcgtgatgacccagactcccctgtccctgcctgtgactcccggagaaccagcctccatttcctgccggtcctcccagtccctgctggacagcgacgacggcaacacttacctggactggtacttgcagaagccgggccaatcgcctcagctgctgatctataccctgtcattccgggcctcaggagtgcctgaccgcttctcgggatcagggagcgggaccgatttcaccctgaaaattaggcgagtggaagccgaggacgtcggagtgtactactgcatgcagcgcatcggcttcccgattacgtttggacagggtacccggcttgagatcaag

SEQ ID NO: 177 - protein, 474 aa, Artificial Sequence
  source: /note=“Description of Artificial Sequence: Synthetic polypeptide”
EVQLVESGGGLVKPGGSLRLSCAASGFTFSSFRMNWVRQAPGKGLEWVSSISSSSSYIYYADSVKGRFTISRDNAKNSLYLQMNSLRAEDTAVYYCARWLSYYGMDVWGQGTTVTVSSGGGGSGGGGSGGGGSGGGGSDIVMTQTPLSLPVTPGEPASISCRSSQSLLDSDDGNTYLDWYLQKPGQSPQLLIYTLSFRASGVPDRFSGSGSGTDFTLKIRRVEAEDVGVYYCMQRIGFPITFGQGTRLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 178 - DNA, 1422 bp, Artificial Sequence
  source: /note=“Description of Artificial Sequence: Synthetic polynucleotide”
gaagtgcaactggtggagagcggtggagggcttgtcaagcccggaggatcgctgcggctgtcctgtgctgcgtccgggttcaccttctcctcgttccgcatgaactgggtcagacaggcaccgggaaagggcctcgaatgggtgtcctcaatctcatcgtcctcgtcctacatctactacgccgactccgtgaaaggccgcttcaccatctcccgggacaacgccaagaactcgctgtacctccaaatgaatagcctcagggcggaagatactgctgtgtattactgcgcacgctggctttcctactacggcatggacgtctggggccaagggaccactgtgaccgtgtctagcggaggcggaggttcagggggcggtggatcaggcggaggaggatcggggggtggtggatcggatatcgtgatgacccagactcccctgtccctgcctgtgactcccggagaaccagcctccatttcctgccggtcctcccagtccctgctggacagcgacgacggcaacacttacctggactggtacttgcagaagccgggccaatcgcctcagctgctgatctataccctgtcattccgggcctcaggagtgcctgaccgcttctcgggatcagggagcgggaccgatttcaccctgaaaattaggcgagtggaagccgaggacgtcggagtgtactactgcatgcagcgcatcggcttcccgattacgtttggacagggtacccggcttgagatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 179 - protein, 5 aa, Artificial Sequence
  source: /note=“Description of Artificial Sequence: Synthetic peptide”
  VARIANT (1)..(1): /replace=“Ser”
  VARIANT (3)..(3): /replace=“Arg”
  VARIANT (5)..(5): /replace=“Asn”
  SITE (1)..(5): /note=“Variant residues given in the sequence have no preference with respect to those in the annotations for variant positions”
GFWMS

SEQ ID NO: 180 - protein, 17 aa, Artificial Sequence
  source: /note=“Description of Artificial Sequence: Synthetic peptide”
  VARIANT (1)..(1): /replace=“Ser”
  VARIANT (3)..(3): /replace=“Ser”
  VARIANT (4)..(4): /replace=“Ser”
  VARIANT (5)..(5): /replace=“Ser”
  VARIANT (6)..(6): /replace=“Ser”
  VARIANT (8)..(8): /replace=“Tyr”
  VARIANT (9)..(9): /replace=“Ile”
  VARIANT (12)..(12): /replace=“Ala”
  VARIANT (16)..(16): /replace=“Lys”
  SITE (1)..(17): /note=“Variant residues given in the sequence have no preference with respect to those in the annotations for variant positions”
NIKQDGSEKYYVDSVRG

SEQ ID NO: 181 - protein, 9 aa, Artificial Sequence
  source: /note=“Description of Artificial Sequence: Synthetic peptide”
  VARIANT (1)..(1): /replace=“Trp”
  VARIANT (3)..(3): /replace=“Ser”
  SITE (1)..(9): /note=“Variant residues given in the sequence have no preference with respect to those in the annotations for variant positions”
ALDYYGMDV

SEQ ID NO: 182 - protein, 7 aa, Artificial Sequence
  source: /note=“Description of Artificial Sequence: Synthetic peptide”
  VARIANT (4)..(4): /replace=“Phe”
  SITE (1)..(7): /note=“Variant residues given in the sequence have no preference with respect to those in the annotations for variant positions”
TLSYRAS

SEQ ID NO: 183 - protein, 10 aa, Artificial Sequence
  source: /note=“Description of Artificial Sequence: Synthetic peptide”
  VARIANT (1)..(1): /replace=“Met”
  VARIANT (4)..(4): /replace=“Ile”
  VARIANT (5)..(5): /replace=“Gly”
  VARIANT (8)..(8): /replace=“ ”
  SITE (1)..(10): /note=“Variant residues given in the sequence have no preference with respect to those in the annotations for variant positions”
TQRLEFPSIT

SEQ ID NO: 184 - protein, 7 aa, Artificial Sequence
  source: /note=“Description of Artificial Sequence: Synthetic peptide”
  VARIANT (6)..(6): /replace=“Ser”
  SITE (1)..(7): /note=“Variant residues given in the sequence have no preference with respect to those in the annotations for variant positions”
GFTFSGF

SEQ ID NO: 185 - protein, 6 aa, Artificial Sequence
  source: /note=“Description of Artificial Sequence: Synthetic peptide”
  VARIANT (1)..(1): /replace=“Ser”
  VARIANT (2)..(2): /replace=“Ser”
  VARIANT (3)..(3): /replace=“Ser”
  VARIANT (4)..(4): /replace=“Ser”
  VARIANT (6)..(6): /replace=“Tyr”
  SITE (1)..(6): /note=“Variant residues given in the sequence have no preference with respect to those in the annotations for variant positions”
KQDGSE

SEQ ID NO: 186 - protein, 7 aa, Artificial Sequence
  source: /note=“Description of Artificial Sequence: Synthetic peptide”
  VARIANT (2)..(2): /replace=“Ile”
  VARIANT (3)..(3): /replace=“Gly”
  VARIANT (6)..(6): /replace=“ ”
  SITE (1)..(7): /note=“Variant residues given in the sequence have no preference with respect to those in the annotations for variant positions”
RLEFPSI

SEQ ID NO: 187 - protein, 8 aa, Artificial Sequence
  source: /note=“Description of Artificial Sequence: Synthetic peptide”
  VARIANT (6)..(6): /replace=“Ser”
  VARIANT (8)..(8): /replace=“Arg”
  SITE (1)..(8): /note=“Variant residues given in the sequence have no preference with respect to those in the annotations for variant positions”
GFTFSGFW

SEQ ID NO: 188 - protein, 8 aa, Artificial Sequence
  source: /note=“Description of Artificial Sequence: Synthetic peptide”
  VARIANT (2)..(2): /replace=“Ser”
  VARIANT (3)..(3): /replace=“Ser”
  VARIANT (4)..(4): /replace=“Ser”
  VARIANT (5)..(5): /replace=“Ser”
  VARIANT (7)..(7): /replace=“Tyr”
  VARIANT (8)..(8): /replace=“Ile”
  SITE (1)..(8): /note=“Variant residues given in the sequence have no preference with respect to those in the annotations for variant positions”
IKQDGSEK

SEQ ID NO: 189 - protein, 11 aa, Artificial Sequence
  source: /note=“Description of Artificial Sequence: Synthetic peptide”
  VARIANT (3)..(3): /replace=“Trp”
  VARIANT (5)..(5): /replace=“Ser”
  SITE (1)..(11): /note=“Variant residues given in the sequence have no preference with respect to those in the annotations for variant positions”
ARALDYYGMDV

SEQ ID NO: 190 - DNA, 521 bp, Unknown
  source: /note=“Description of Unknown: PGK Promoter sequence”
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccgcctcgtccttcgcagcggccccccgggtgttcccatcgccgcttctaggcccactgcgacgcttgcctgcacttcttacacgctctgggtcccagccgcggcgacgcaaagggccttggtgcgggtctcgtcggcgcagggacgcgtttgggtcccgacggaaccttttccgcgttggggttggggcaccataagct

SEQ ID NO: 191 - DNA, 221 bp, Artificial Sequence
  source: /note=“Description of Artificial Sequence: Synthetic polynucleotide”
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacg

SEQ ID NO: 192 - DNA, 324 bp, Artificial Sequence
  source: /note=“Description of Artificial Sequence: Synthetic polynucleotide”
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccg

SEQ ID NO: 193 - DNA, 422 bp, Artificial Sequence
  source: /note=“Description of Artificial Sequence: Synthetic polynucleotide”
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccgcctcgtccttcgcagcggccccccgggtgttcccatcgccgcttctaggcccactgcgacgcttgcctgcacttcttacacgctctgggtcccagccg

SEQ ID NO: 194-197 - skipped (4 entries)

SEQ ID NO: 198 - DNA, 118 bp, Artificial Sequence
  source: /note=“Description of Artificial Sequence: Synthetic polynucleotide”
acccctctctccagccactaagccagttgctccctcggctgacggctgcacgcgaggcctccgaacgtcttacgccttgtggcgcgcccgtccttgtcccgggtgtgatggcggggtg

SEQ ID NO: 199 - DNA, 63 bp, Artificial Sequence
  source: /note=“Description of Artificial Sequence: Synthetic oligonucleotide”
atggccctccctgtcaccgccctgctgcttccgctggctcttctgctccacgccgctcggccc

SEQ ID NO: 200-201 - skipped (2 entries)

SEQ ID NO: 202 - protein, 69 aa, Artificial Sequence
  source: /note=“Description of Artificial Sequence: Synthetic polypeptide”
TTTPAPRPPTPAPTIASQPLSLRPEACRPAAGGAVHTRGLDFACDIYIWAPLAGTCGVLLLSLVITLYC

SEQ ID NO: 203-251 - skipped (49 entries)

SEQ ID NO: 252 - DNA, 63 bp, Artificial Sequence
  source: /note=“Description of Artificial Sequence: Synthetic oligonucleotide”
atggccctccctgtcaccgctctgttgctgccgcttgctctgctgctccacgcagcgcgaccg

SEQ ID NO: 253 - DNA, 747 bp, Artificial Sequence
  source: /note=“Description of Artificial Sequence: Synthetic polynucleotide”
caggtacaattgcaggagtctggaggcggtgtggtgcaacccggtcgcagcttgcgcctgagttgtgctgcgtctggatttacattttcatcttacggaatgcattgggtacgccaggcaccggggaaaggccttgaatgggtggctgtaatttcatacgatggttccaacaaatactatgctgactcagtcaagggtcgatttacaattagtcgggacaactccaagaacaccctttatcttcaaatgaattcccttagagcagaggatacggcggtctattactgtggtggcagtggttatgcacttcatgatgattactatggcttggatgtctgggggcaagggacgcttgtaactgtatcctctggtggtggtggtagtggtgggggaggctccggcggtggcggctctcaatctgctctgactcaaccagcaagcgtatcagggtcaccgggacagagtattaccataagttgcacggggacctctagcgatgtaggggggtataattatgtatcttggtatcaacaacaccccgggaaagcccctaaattgatgatctacgacgtgagcaatcgacctagtggcgtatcaaatcgcttctctggtagcaagagtgggaatacggcgtcccttactattagcggattgcaagcagaagatgaggccgattactactgcagctcctatactagctcttctacattgtacgtctttgggagcggaacaaaagtaacagtactc

SEQ ID NO: 254 - DNA, 207 bp, Artificial Sequence
  source: /note=“Description of Artificial Sequence: Synthetic polynucleotide”
acaacaacacctgccccgagaccgcctacaccagccccgactattgccagccagcctctgagcctcaggcctgaggcctgtaggcccgcagcgggcggcgcagttcatacacggggcttggatttcgcttgtgatatttatatttgggctcctttggcggggacatgtggcgtgctgcttctgtcacttgttattacactgtactgt

SEQ ID NO: 255 - DNA, 126 bp, Artificial Sequence
  source: /note=“Description of Artificial Sequence: Synthetic polynucleotide”
aaacgcgggcgaaaaaaattgctgtatatttttaagcagccatttatgaggcccgttcagacgacgcaggaggaggacggttgctcttgcaggttcccagaagaggaagaagggggctgtgaattg

SEQ ID NO: 256 - DNA, 336 bp, Artificial Sequence
  source: /note=“Description of Artificial Sequence: Synthetic polynucleotide”
cgggttaaattttcaagatccgcagacgctccagcataccaacagggacaaaaccaactctataacgagctgaatcttggaagaagggaggaatatgatgtgctggataaacggcgcggtagagatccggagatgggcggaaaaccaaggcgaaaaaaccctcaggagggactctacaacgaactgcagaaagacaaaatggcggaggcttattccgaaataggcatgaagggcgagcggaggcgagggaaagggcacgacggactgtatcaaggcctctcaaccgcgactaaggatacgtacgacgccctgcacatgcaggccctgcctccgaga

SEQ ID NO: 257 - protein, 493 aa, Artificial Sequence
  source: /note=“Description of Artificial Sequence: Synthetic polypeptide”
MALPVTALLLPLALLLHAARPQVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCGGSGYALHDDYYGLDVWGQGTLVTVSSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGSGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 258 - DNA, 1479 bp, Artificial Sequence
  source: /note=“Description of Artificial Sequence: Synthetic polynucleotide”
atggccctccctgtcaccgctctgttgctgccgcttgctctgctgctccacgcagcgcgaccgcaggtacaattgcaggagtctggaggcggtgtggtgcaacccggtcgcagcttgcgcctgagttgtgctgcgtctggatttacattttcatcttacggaatgcattgggtacgccaggcaccggggaaaggccttgaatgggtggctgtaatttcatacgatggttccaacaaatactatgctgactcagtcaagggtcgatttacaattagtcgggacaactccaagaacaccctttatcttcaaatgaattcccttagagcagaggatacggcggtctattactgtggtggcagtggttatgcacttcatgatgattactatggcttggatgtctgggggcaagggacgcttgtaactgtatcctctggtggtggtggtagtggtgggggaggctccggcggtggcggctctcaatctgctctgactcaaccagcaagcgtatcagggtcaccgggacagagtattaccataagttgcacggggacctctagcgatgtaggggggtataattatgtatcttggtatcaacaacaccccgggaaagcccctaaattgatgatctacgacgtgagcaatcgacctagtggcgtatcaaatcgcttctctggtagcaagagtgggaatacggcgtcccttactattagcggattgcaagcagaagatgaggccgattactactgcagctcctatactagctcttctacattgtacgtctttgggagcggaacaaaagtaacagtactcacaacaacacctgccccgagaccgcctacaccagccccgactattgccagccagcctctgagcctcaggcctgaggcctgtaggcccgcagcgggcggcgcagttcatacacggggcttggatttcgcttgtgatatttatatttgggctcctttggcggggacatgtggcgtgctgcttctgtcacttgttattacactgtactgtaaacgcgggcgaaaaaaattgctgtatatttttaagcagccatttatgaggcccgttcagacgacgcaggaggaggacggttgctcttgcaggttcccagaagaggaagaagggggctgtgaattgcgggttaaattttcaagatccgcagacgctccagcataccaacagggacaaaaccaactctataacgagctgaatcttggaagaagggaggaatatgatgtgctggataaacggcgcggtagagatccggagatgggcggaaaaccaaggcgaaaaaaccctcaggagggactctacaacgaactgcagaaagacaaaatggcggaggcttattccgaaataggcatgaagggcgagcggaggcgagggaaagggcacgacggactgtatcaaggcctctcaaccgcgactaaggatacgtacgacgccctgcacatgcaggccctgcctccgaga

SEQ ID NO: 259-274 - skipped (16 entries)

SEQ ID NO: 275 - protein, 132 aa, Artificial Sequence
  source: /note=“Description of Artificial Sequence: Synthetic polypeptide”
DVPDYASLGGPSSPKKKRKVSRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETSY

SEQ ID NO: 276 - protein, 108 aa, Artificial Sequence
  source: /note=“Description of Artificial Sequence: Synthetic polypeptide”
VQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETS

SEQ ID NO: 277 - protein, 93 aa, Homo sapiens [sequence omitted]

SEQ ID NO: 278 - protein, 95 aa, Artificial Sequence
  source: /note=“Description of Artificial Sequence: Synthetic polypeptide”
ILWHEMWHEGLIEASRLYFGERNVKGMFEVLEPLHAMMERGPQTLKETSFNQAYGRDLMEAQEWCRKYMKSGNVKDLTQAWDLYYHVFRRISKTS

SEQ ID NO: 279 - protein, 95 aa, Artificial Sequence
  source: /note=“Description of Artificial Sequence: Synthetic polypeptide”
ILWHEMWHEGLLEASRLYFGERNVKGMFEVLEPLHAMMERGPQTLKETSFNQAYGRDLMEAQEWCRKYMKSGNVKDLTQAWDLYYHVFRRISKTS

SEQ ID NO: 280 - protein, 95 aa, Artificial Sequence
  source: /note=“Description of Artificial Sequence: Synthetic polypeptide”
ILWHEMWHEGLEEASRLYFGERNVKGMFEVLEPLHAMMERGPQTLKETSFNQAYGRDLMEAQEWCRKYMKSGNVKDLLQAWDLYYHVFRRISKTS

SEQ ID NO: 281 - protein, 95 aa, Artificial Sequence
  source: /note=“Description of Artificial Sequence: Synthetic polypeptide”
  MOD_RES (12)..(12): Any amino acid
  MOD_RES (78)..(78): Any amino acid
ILWHEMWHEGLXEASRLYFGERNVKGMFEVLEPLHAMMERGPQTLKETSFNQAYGRDLMEAQEWCRKYMKSGNVKDLXQAWDLYYHVFRRISKTS

SEQ ID NO: 282 - protein, 95 aa, Artificial Sequence
  source: /note=“Description of Artificial Sequence: Synthetic polypeptide”
ILWHEMWHEGLIEASRLYFGERNVKGMFEVLEPLHAMMERGPQTLKETSFNQAYGRDLMEAQEWCRKYMKSGNVKDLLQAWDLYYHVFRRISKTS

SEQ ID NO: 283 - protein, 95 aa, Artificial Sequence
  source: /note=“Description of Artificial Sequence: Synthetic polypeptide”
ILWHEMWHEGLLEASRLYFGERNVKGMFEVLEPLHAMMERGPQTLKETSFNQAYGRDLMEAQEWCRKYMKSGNVKDLLQAWDLYYHVFRRISKTS

SEQ ID NO: 284 - protein, 1132 aa, Homo sapiens [sequence omitted]

SEQ ID NO: 285 - protein, 253 aa, Artificial Sequence
  source: /note=“Description of Artificial Sequence: Synthetic polypeptide”
EVQLQQSGPGLVKPSQTLSLTCAISGDSMLSNSDTWNWIRQSPSRGLEWLGRTYHRSTWYDDYASSVRGRVSINVDTSKNQYSLQLNAVTPEDTGVYYCARVRLQDGNSWSDAFDVWGQGTMVTVSSGGGGSGGGGSGGGGSQSALTQPASASGSPGQSVTISCTGTSSDVGGYNYVSWYQQHPGKAPKLMIYDVSNRPSGVSNRFSGSKSGNTASLTISGLQAEDEADYYCSSYTSSSTLYVFGTGTQLTVL

SEQ ID NO: 286 - protein, 127 aa, Artificial Sequence
  source: /note=“Description of Artificial Sequence: Synthetic polypeptide”
EVQLQQSGPGLVKPSQTLSLTCAISGDSMLSNSDTWNWIRQSPSRGLEWLGRTYHRSTWYDDYASSVRGRVSINVDTSKNQYSLQLNAVTPEDTGVYYCARVRLQDGNSWSDAFDVWGQGTMVTVSS

SEQ ID NO: 287 - protein, 111 aa, Artificial Sequence
  source: /note=“Description of Artificial Sequence: Synthetic polypeptide”
QSALTQPASASGSPGQSVTISCTGTSSDVGGYNYVSWYQQHPGKAPKLMIYDVSNRPSGVSNRFSGSKSGNTASLTISGLQAEDEADYYCSSYTSSSTLYVFGTGTQLTVL

SEQ ID NO: 288 - protein, 12 aa, Artificial Sequence
  source: /note=“Description of Artificial Sequence: Synthetic peptide”
GDSMLSNSDTWN

SEQ ID NO: 289 - protein, 7 aa, Artificial Sequence
  source: /note=“Description of Artificial Sequence: Synthetic peptide”
SNSDTWN

SEQ ID NO: 290 - protein, 18 aa, Artificial Sequence
  source: /note=“Description of Artificial Sequence: Synthetic peptide”
RTYHRSTWYDDYASSVRG

SEQ ID NO: 291 - protein, 15 aa, Artificial Sequence
  source: /note=“Description of Artificial Sequence: Synthetic peptide”
VRLQDGNSWSDAFDV

SEQ ID NO: 292 - protein, 465 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 293 - protein, 242 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 294 - protein, 465 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 295 - protein, 5 aa, Artificial Sequence
  source: /note=“Description of Artificial Sequence: Synthetic peptide”
DYGVS

SEQ ID NO: 296 - protein, 16 aa, Artificial Sequence
  source: /note=“Description of Artificial Sequence: Synthetic peptide”
VIWGSETTYYNSALKS

SEQ ID NO: 297 - protein, 12 aa, Artificial Sequence
  source: /note=“Description of Artificial Sequence: Synthetic peptide”
HYYYGGSYAMDY

SEQ ID NO: 298 - protein, 11 aa, Artificial Sequence
  source: /note=“Description of Artificial Sequence: Synthetic peptide”
RASQDISKYLN

SEQ ID NO: 299 - protein, 7 aa, Artificial Sequence
  source: /note=“Description of Artificial Sequence: Synthetic peptide”
HTSRLHS

SEQ ID NO: 300 - protein, 9 aa, Artificial Sequence
  source: /note=“Description of Artificial Sequence: Synthetic peptide”
QQGNTLPYT

SEQ ID NO: 301 - protein, 486 aa, Artificial Sequence
  source: /note=“Description of Artificial Sequence: Synthetic polypeptide”
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 302 - DNA, 1458 bp, Artificial Sequence
  source: /note=“Description of Artificial Sequence: Synthetic polynucleotide”
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 303 - protein, 242 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 304 - protein, 16 aa, Artificial Sequence
  source: /note=“Description of Artificial Sequence: Synthetic peptide”
VIWGSETTYYQSSLKS

SEQ ID NO: 305 - DNA, 813 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagccaccaccatcatcaccatcaccat

SEQ ID NO: 306 - protein, 486 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 307 - DNA, 1458 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 308 - protein, 7 aa, Artificial Sequence
  source: /note=“Description of Artificial Sequence: Synthetic peptide”
LAEAAAK

SEQ ID NO: 309 - protein, 284 aa, Artificial Sequence
  source: /note=“Description of Artificial Sequence: Synthetic polypeptide”
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 310 - protein, 60 aa, Artificial Sequence
  source: /note=“Description of Artificial Sequence: Synthetic polypeptide”
MHKRSHTGERPFQCNQCGASFTQKGNLLRHIKLHTGEKPFKCHLCNTASAEARHIKAEMG

SEQ ID NO: 311 - protein, 60 aa, Artificial Sequence
  source: /note=“Description of Artificial Sequence: Synthetic polypeptide”
MHRRSHTGERPFQCNQCGASFTQRGNLLRHIRLHTGERPFRCHLCNTASAEARHIRAEMG

SEQ ID NO: 312 - protein, 59 aa, Artificial Sequence
  source: /note=“Description of Artificial Sequence: Synthetic polypeptide”
HKRSHTGERPFQCNQCGASFTQKGNLLRHIKLHTGEKPFKCHLCNTASAEARHIKAEMG

SEQ ID NO: 313 - protein, 59 aa, Artificial Sequence
  source: /note=“Description of Artificial Sequence: Synthetic polypeptide”
HRRSHTGERPFQCNQCGASFTQRGNLLRHIRLHTGERPFRCHLCNTASAEARHIRAEMG

SEQ ID NO: 314 - protein, 45 aa, Artificial Sequence
  source: /note=“Description of Artificial Sequence: Synthetic polypeptide”
HKRSHTGERPFQCNQCGASFTQKGNLLRHIKLHTGEKPFKCHLCN

SEQ ID NO: 315 - protein, 35 aa, Artificial Sequence
  source: /note=“Description of Artificial Sequence: Synthetic polypeptide”
HKRSHTGERPFQCNQCGASFTQKGNLLRHIKLHTG

SEQ ID NO: 316 - protein, 31 aa, Artificial Sequence
  source: /note=“Description of Artificial Sequence: Synthetic polypeptide”
HTGERPFQCNQCGASFTQKGNLLRHIKLHTG

SEQ ID NO: 317 - protein, 23 aa, Artificial Sequence
  source: /note=“Description of Artificial Sequence: Synthetic peptide”
TGERPFQCNQCGASFTQKGNLLR

SEQ ID NO: 318 - protein, 26 aa, Artificial Sequence
  source: /note=“Description of Artificial Sequence: Synthetic peptide”
PFQCNQCGASFTQKGNLLRHIKLHTG

SEQ ID NO: 319 - protein, 11 aa, Artificial Sequence
  source: /note=“Description of Artificial Sequence: Synthetic peptide”
PFQCNQCGASF

SEQ ID NO: 320 - protein, 14 aa, Artificial Sequence
  source: /note=“Description of Artificial Sequence: Synthetic peptide”
TASAEARHIKAEMG

SEQ ID NO: 321 - protein, 60 aa, Artificial Sequence
  source: /note=“Description of Artificial Sequence: Synthetic polypeptide”
MHKRSHTGERPFQCNQCGASFTQKGNLLRHIKLHTGEKPFKCHLCNTASAEARHIRAEMG

SEQ ID NO: 322 - protein, 60 aa, Artificial Sequence
  source: /note=“Description of Artificial Sequence: Synthetic polypeptide”
MHKRSHTGERPFQCNQCGASFTQKGNLLRHIKLHTGEKPFKCHLCNTASAEARHISAEMG

SEQ ID NO: 323 - protein, 60 aa, Artificial Sequence
  source: /note=“Description of Artificial Sequence: Synthetic polypeptide”
MHKRSHTGERPFQCNQCGASFTQKGNLLRHIKLHTGEKPFKCHLCNMALEKMALEKMALE

SEQ ID NO: 324 - protein, 50 aa, Artificial Sequence
  source: /note=“Description of Artificial Sequence: Synthetic polypeptide”
MHKRSHTGERPFQCNQCGASFTQKGNLLRHIKLHTGMALEKMALEKMALE

SEQ ID NO: 325 - protein, 46 aa, Artificial Sequence
  source: /note=“Description of Artificial Sequence: Synthetic polypeptide”
MHTGERPFQCNQCGASFTQKGNLLRHIKLHTGMALEKMALEKMALE

SEQ ID NO: 326 - protein, 38 aa, Artificial Sequence
  source: /note=“Description of Artificial Sequence: Synthetic polypeptide”
MTGERPFQCNQCGASFTQKGNLLRMALEKMALEKMALE

SEQ ID NO: 327 - protein, 26 aa, Artificial Sequence
  source: /note=“Description of Artificial Sequence: Synthetic peptide”
MPFQCNQCGASFMALEKMALEKMALE

SEQ ID NO: 328 - protein, 509 aa, Homo sapiens
MEDIQTNAELKSTQEQSVPAESAAVLNDYSLTKSHEMENVDSGEGPANEDEDIGDDSMKVKDEYSERDENVLKSEPMGNAEEPEIPYSYSREYNEYENIKLERHVVSFDSSRPTSGKMNCDVCGLSCISFNVLMVHKRSHTGERPFQCNQCGASFTQKGNLLRHIKLHTGEKPFKCHLCNYACQRRDALTGHLRTHSVEKPYKCEFCGRSYKQRSSLEEHKERCRTFLQSTDPGDTASAEARHIKAEMGSERALVLDRLASNVAKRKSSMPQKFIGEKRHCFDVNYNSSYMYEKESELIQTRMMDQAINNAISYLGAEALRPLVQTPPAPTSEMVPVISSMYPIALTRAEMSNGAPQELEKKSIHLPEKSVPSERGLSPNNSGHDSTDTDSNHEERQNHIYQQNHMVLSRARNGMPLLKEVPRSYELLKPPPICPRDSVKVINKEGEVMDVYRCDHCRVLFLDYVMFTIHMGCHGFRDPFECNMCGYRSHDRYEFSSHIARGEHRALLK

SEQ ID NO: 329 - protein, 519 aa, Homo sapiens
MDADEGQDMSQVSGKESPPVSDTPDEGDEPMPIPEDLSTTSGGQQSSKSDRVVASNVKVETQSDEENGRACEMNGEECAEDLRMLDASGEKMNGSHRDQGSSALSGVGGIRLPNGKLKCDICGIICIGPNVLMVHKRSHTGERPFQCNQCGASFTQKGNLLRHIKLHSGEKPFKCHLCNYACRRRDALTGHLRTHSVGKPHKCGYCGRSYKQRSSLEEHKERCHNYLESMGLPGTLYPVIKEETNHSEMAEDLCKIGSERSLVLDRLASNVAKRKSSMPQKFLGDKGLSDTPYDSSASYEKENEMMKSHVMDQAINNAINYLGAESLRPLVQTPPGGSEVVPVISPMYQLHKPLAEGTPRSNHSAQDSAVENLLLLSKAKLVPSEREASPSNSCQDSTDTESNNEEQRSGLIYLTNHIAPHARNGLSLKEEHRAYDLLRAASENSQDALRVVSTSGEQMKVYKCEHCRVLFLDHVMYTIHMGCHGFRDPFECNMCGYHSQDRYEFSSHITRGEHRFHMS

SEQ ID NO: 330 - protein, 526 aa, Homo sapiens [sequence omitted]

SEQ ID NO: 331 - protein, 585 aa, Homo sapiens
MHTPPALPRRFQGGGRVRTPGSHRQGKDNLERDPSGGCVPDFLPQAQDSNHFIMESLFCESSGDSSLEKEFLGAPVGPSVSTPNSQHSSPSRSLSANSIKVEMYSDEESSRLLGPDERLLEKDDSVIVEDSLSEPLGYCDGSGPEPHSPGGIRLPNGKLKCDVCGMVCIGPNVLMVHKRSHTGERPFHCNQCGASFTQKGNLLRHIKLHSGEKPFKCPFCNYACRRRDALTGHLRTHSVSSPTVGKPYKCNYCGRSYKQQSTLEEHKERCHNYLQSLSTEAQALAGQPGDEIRDLEMVPDSMLHSSSERPTFIDRLANSLTKRKRSTPQKFVGEKQMRFSLSDLPYDVNSGGYEKDVELVAHHSLEPGFGSSLAFVGAEHLRPLRLPPTNCISELTPVISSVYTQMQPLPGRLELPGSREAGEGPEDLADGGPLLYRPRGPLTDPGASPSNGCQDSTDTESNHEDRVAGVVSLPQGPPPQPPPTIVVGRHSPAYAKEDPKPQEGLLRGTPGPSKEVLRVVGESGEPVKAFKCEHCRILFLDHVMFTIHMGCHGFRDPFECNICGYHSQDRYEFSSHIVRGEHKVG

SEQ ID NO: 332 - protein, 419 aa, Homo sapiens
MGEKKPEPLDFVKDFQEYLTQQTHHVNMISGSVSGDKEAEALQGAGTDGDQNGLDHPSVEVSLDENSGMLVDGFERTFDGKLKCRYCNYASKGTARLIEHIRIHTGEKPHRCHLCPFASAYERHLEAHMRSHTGEKPYKCELCSFRCSDRSNLSHHRRRKHKMVPIKGTRSSLSSKKMWGVLQKKTSNLGYSRRALINLSPPSMVVQKPDYLNDFTHEIPNIQTDSYESMAKTTPTGGLPRDPQELMVDNPLNQLSTLAGQLSSLPPENQNPASPDVVPCPDEKPFMIQQPSTQAVVSAVSASIPQSSSPTSPEPRPSHSQRNYSPVAGPSSEPSAHTSTPSIGNSQPSTPAPALPVQDPQLLHHCQHCDMYFADNILYTIHMGCHGYENPFQCNICGCKCKNKYDFACHFARGQHNQH

SEQ ID NO: 333 - protein, 30 aa, Artificial Sequence
  source: /note=“Description of Artificial Sequence: Synthetic polypeptide”
HTGERPFQCNQCGASFTQKGNLLRHIKLHT

SEQ ID NO: 334 - protein, 46 aa, Artificial Sequence
  source: /note=“Description of Artificial Sequence: Synthetic polypeptide”
MHKRSHTGERPFHCNQCGASFTQKGNLLRHIKLHTGEKPFKCHLCN

SEQ ID NO: 335 - protein, 16 aa, Artificial Sequence
  source: /note=“Description of Artificial Sequence: Synthetic peptide”
GGGGSGGGGTGGGGSG

SEQ ID NO: 336 - protein, 8 aa, Artificial Sequence
  source: /note=“Description of Artificial Sequence: Synthetic peptide”
SARNRQKR

SEQ ID NO: 337 - protein, 45 aa, Artificial Sequence
  source: /note=“Description of Artificial Sequence: Synthetic polypeptide”
HRRSHTGERPFQCNQCGASFTQRGNLLRHIRLHTGERPFRCHLCN

SEQ ID NO: 338 - protein, 35 aa, Artificial Sequence
  source: /note=“Description of Artificial Sequence: Synthetic polypeptide”
HRRSHTGERPFQCNQCGASFTQRGNLLRHIRLHTG

SEQ ID NO: 339 - protein, 14 aa, Artificial Sequence
  source: /note=“Description of Artificial Sequence: Synthetic peptide”
TASAEARHIRAEMG

SEQ ID NO: 340 - protein, 245 aa, Homo sapiens
SLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRL

SEQ ID NO: 341 - DNA, 735 bp, Homo sapiens
tcgttggcactttccctgactgccgaccagatggtgtccgcccttctggacgccgagcctccaattctgtactcggagtacgatccgactcgcccgttctccgaagccagcatgatgggcctgttgactaacctggcggaccgcgagttggtgcacatgattaactgggctaagcgggtgccgggcttcgtggacctgactctgcacgaccaagtgcacctcctggaatgcgcctggctggaaatcctcatgatcggcctcgtgtggagatccatggagcatcccggaaagctcctgtttgcacccaacctcctgcttgatcgcaaccagggaaaatgcgtggaagggatggtcgagattttcgacatgctgctcgccacctcttcccggttccggatgatgaatctgcagggagaagagttcgtgtgtctgaagtcaatcatcctgctgaactccggggtctataccttcctgagctcgaccctcaagtcactggaggaaaaagaccacatccatcgcgtgctcgataagatcaccgacacccttatccatctcatggcgaaggctggactgaccctgcaacagcagcaccagaggctggcccagttgctgctgattctgagccacatccggcacatgtcgaacaaggggatggaacacctgtacagcatgaagtgcaagaacgtcgtgcctctgtacgatctgctcctggaaatgctggacgcgcacagactc

SEQ ID NO: 342 - protein, 245 aa, Artificial Sequence
  source: /note=“Description of Artificial Sequence: Synthetic polypeptide”
SLALSLTADQMVSALLDAEPPILYSEYDPTRPFSEASMMGLLTNLADRELVHMINWAKRVPGFVDLALHDQVHLLECAWMEILMIGLVWRSMEHPGKLLFAPNLLLDRNQGKCVEGGVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSSKRMEHLYSMKCKNVVPLSDLLLEMLDAHRL

SEQ ID NO: 343 - DNA, 735 bp, Artificial Sequence
  source: /note=“Description of Artificial Sequence: Synthetic polynucleotide”
tcgttggcactttccctgactgccgaccagatggtgtccgcccttctggacgccgagcctccaattctgtactcggagtacgatccgactcgcccgttctccgaagccagcatgatgggcctgttgactaacctggcggaccgcgagttggtgcacatgattaactgggctaagcgggtgccgggcttcgtggacctggccctgcacgaccaagtgcacctcctggaatgcgcctggatggaaatcctcatgatcggcctcgtgtggagatccatggagcatcccggaaagctcctgtttgcacccaacctcctgcttgatcgcaaccagggaaaatgcgtggaagggggtgtcgagattttcgacatgctgctcgccacctcttcccggttccggatgatgaatctgcagggagaagagttcgtgtgtctgaagtcaatcatcctgctgaactccggggtctataccttcctgagctcgaccctcaagtcactggaggaaaaagaccacatccatcgcgtgctcgataagatcaccgacacccttatccatctcatggcgaaggctggactgaccctgcaacagcagcaccagaggctggcccagttgctgctgattctgagccacatccggcacatgtcgtccaagaggatggaacacctgtacagcatgaagtgcaagaacgtcgtgcctctgtccgatctgctcctggaaatgctggacgcgcacagactc

SEQ ID NO: 344 - protein, 245 aa, Artificial Sequence
  source: /note=“Description of Artificial Sequence: Synthetic polypeptide”
SLALSLTADQMVSALLDAEPPILYSEYDPTRPFSEASMMGLLTNLADRELVHMINWAKRVPGFVDLTLHDQVHLLECAWMEILMIGLVWRSMEHPGKLLFAPNLLLDRNQGKCVEGGVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKRMEHLYSMKCKNVVPLSDLLLEMLDAHRL

SEQ ID NO: 345 - DNA, 735 bp, Artificial Sequence
  source: /note=“Description of Artificial Sequence: Synthetic polynucleotide”
tcgttggcactttccctgactgccgaccagatggtgtccgcccttctggacgccgagcctccaattctgtactcggagtacgatccgactcgcccgttctccgaagccagcatgatgggcctgttgactaacctggcggaccgcgagttggtgcacatgattaactgggctaagcgggtgccgggcttcgtggacctgaccctgcacgaccaagtgcacctcctggaatgcgcctggatggaaatcctcatgatcggcctcgtgtggagatccatggagcatcccggaaagctcctgtttgcacccaacctcctgcttgatcgcaaccagggaaaatgcgtggaagggggtgtcgagattttcgacatgctgctcgccacctcttcccggttccggatgatgaatctgcagggagaagagttcgtgtgtctgaagtcaatcatcctgctgaactccggggtctataccttcctgagctcgaccctcaagtcactggaggaaaaagaccacatccatcgcgtgctcgataagatcaccgacacccttatccatctcatggcgaaggctggactgaccctgcaacagcagcaccagaggctggcccagttgctgctgattctgagccacatccggcacatgtcgaacaagaggatggaacacctgtacagcatgaagtgcaagaacgtcgtgcctctgtccgatctgctcctggaaatgctggacgcgcacagactc

SEQ ID NO: 346 - protein, 107 aa, Artificial Sequence
  source: /note=“Description of Artificial Sequence: Synthetic polypeptide”
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPE

SEQ ID NO: 347 - protein, 158 aa, Artificial Sequence
  source: /note=“Description of Artificial Sequence: Synthetic polypeptide”
ISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 348 - protein, 4 aa, Artificial Sequence
  source: /note=“Description of Artificial Sequence: Synthetic peptide”
  MOD_RES (2)..(2): Any amino acid
  VARIANT (3)..(3): /replace=“Arg”
  SITE (1)..(4): /note=“Variant residues given in the sequence have no preference with respect to those in the annotations for variant positions”
RXKR

SEQ ID NO: 349 - protein, 6 aa, Artificial Sequence
  source: /note=“Description of Artificial Sequence: Synthetic peptide”
  MOD_RES (2)..(4): Any amino acid
  VARIANT (5)..(5): /replace=“Arg”
  SITE (1)..(6): /note=“Variant residues given in the sequence have no preference with respect to those in the annotations for variant positions”
RXXXKR

SEQ ID NO: 350 - protein, 3 aa, Artificial Sequence
  source: /note=“Description of Artificial Sequence: Synthetic peptide”
  MOD_RES (3)..(3): Any amino acid
  SITE (1)..(3): /note=“Variant residues given in the sequence have no preference with respect to those in the annotations for variant positions”
RRX

SEQ ID NO: 351 - protein, 5 aa, Artificial Sequence
  source: /note=“Description of Artificial Sequence: Synthetic peptide”
  MOD_RES (5)..(5): Any amino acid
  SITE (1)..(5): /note=“Variant residues given in the sequence have no preference with respect to those in the annotations for variant positions”
IEPDX

SEQ ID NO: 352 - protein, 4 aa, Artificial Sequence
  source: /note=“Description of Artificial Sequence: Synthetic peptide”
  VARIANT (2)..(2): /replace=“Asp”
  SITE (1)..(4): /note=“Variant residues given in the sequence have no preference with respect to those in the annotations for variant positions”
IEGR

SEQ ID NO: 353 - protein, 5 aa, Artificial Sequence
  source: /note=“Description of Artificial Sequence: Synthetic peptide”
DDDDK

SEQ ID NO: 354 - protein, 6 aa, Artificial Sequence
  source: /note=“Description of Artificial Sequence: Synthetic peptide”
PGAAHY

SEQ ID NO: 355 - protein, 5 aa, Artificial Sequence
  source: /note=“Description of Artificial Sequence: Synthetic peptide”
  MOD_RES (3)..(3): Any amino acid
  VARIANT (5)..(5): /replace=“Ala”
  SITE (1)..(5): /note=“Variant residues given in the sequence have no preference with respect to those in the annotations for variant positions”
LPXTG

SEQ ID NO: 356 - protein, 7 aa, Artificial Sequence
  source: /note=“Description of Artificial Sequence: Synthetic peptide”
LEVFQGP

SEQ ID NO: 357 - protein, 6 aa, Artificial Sequence
  source: /note=“Description of Artificial Sequence: Synthetic peptide”
LVPRGS

SEQ ID NO: 358 - protein, 7 aa, Artificial Sequence
  source: /note=“Description of Artificial Sequence: Synthetic peptide”
ENLYFQG

SEQ ID NO: 359 - protein, 2 aa, Artificial Sequence
  source: /note=“Description of Artificial Sequence: Synthetic peptide”
  VARIANT (1)..(1): /replace=“Gly” or “Ser” or “Val”
  MOD_RES (2)..(2): Any amino acid
  SITE (1)..(2): /note=“Variant residues given in the sequence have no preference with respect to those in the annotations for variant positions”
AX

SEQ ID NO: 360 - protein, 46 aa, Artificial Sequence
  source: /note=“Description of Artificial Sequence: Synthetic polypeptide”
MHKRSHTGERPFQCNQCGASFTQKGNLLRHIKLHTGEKPFKCHLCN

SEQ ID NO: 361 - protein, 36 aa, Artificial Sequence
  source: /note=“Description of Artificial Sequence: Synthetic polypeptide”
MHKRSHTGERPFQCNQCGASFTQKGNLLRHIKLHTG

SEQ ID NO: 362 - protein, 32 aa, Artificial Sequence
  source: /note=“Description of Artificial Sequence: Synthetic polypeptide”
MHTGERPFQCNQCGASFTQKGNLLRHIKLHTG

SEQ ID NO: 363 - protein, 31 aa, Artificial Sequence
  source: /note=“Description of Artificial Sequence: Synthetic polypeptide”
MHTGERPFQCNQCGASFTQKGNLLRHIKLHT

SEQ ID NO: 364 - protein, 24 aa, Artificial Sequence
  source: /note=“Description of Artificial Sequence: Synthetic peptide”
MTGERPFQCNQCGASFTQKGNLLR

SEQ ID NO: 365 - protein, 27 aa, Artificial Sequence
  source: /note=“Description of Artificial Sequence: Synthetic peptide”
MPFQCNQCGASFTQKGNLLRHIKLHTG

SEQ ID NO: 366 - protein, 12 aa, Artificial Sequence
  source: /note=“Description of Artificial Sequence: Synthetic peptide”
MPFQCNQCGASF

SEQ ID NO: 367 - protein, 59 aa, Artificial Sequence
  source: /note=“Description of Artificial Sequence: Synthetic polypeptide”
HKRSHTGERPFQCNQCGASFTQKGNLLRHIKLHTGEKPFKCHLCNTASAEARHIRAEMG

SEQ ID NO: 368 - protein, 59 aa, Artificial Sequence
  source: /note=“Description of Artificial Sequence: Synthetic polypeptide”
HKRSHTGERPFQCNQCGASFTQKGNLLRHIKLHTGEKPFKCHLCNMALEKMALEKMALE

SEQ ID NO: 369 - protein, 49 aa, Artificial Sequence
  source: /note=“Description of Artificial Sequence: Synthetic polypeptide”
HKRSHTGERPFQCNQCGASFTQKGNLLRHIKLHTGMALEKMALEKMALE

SEQ ID NO: 370 - protein, 45 aa, Artificial Sequence
  source: /note=“Description of Artificial Sequence: Synthetic polypeptide”
HTGERPFQCNQCGASFTQKGNLLRHIKLHTGMALEKMALEKMALE

SEQ ID NO: 371 - protein, 37 aa, Artificial Sequence
  source: /note=“Description of Artificial Sequence: Synthetic polypeptide”
TGERPFQCNQCGASFTQKGNLLRMALEKMALEKMALE

SEQ ID NO: 372 - protein, 25 aa, Artificial Sequence
  source: /note=“Description of Artificial Sequence: Synthetic peptide”
PFQCNQCGASFMALEKMALEKMALE

SEQ ID NO: 373 - protein, 45 aa, Artificial Sequence
  source: /note=“Description of Artificial Sequence: Synthetic polypeptide”
HKRSHTGERPFHCNQCGASFTQKGNLLRHIKLHTGEKPFKCHLCN

SEQ ID NO: 374 - protein, 59 aa, Artificial Sequence
  source: /note=“Description of Artificial Sequence: Synthetic polypeptide”
HKRSHTGERPFQCNQCGASFTQKGNLLRHIKLHTGEKPFKCHLCNTASAEARHISAEMG

SEQ ID NO: 375 - protein, 59 aa, Artificial Sequence
  source: /note=“Description of Artificial Sequence: Synthetic polypeptide”
HKRSHTGERPFHCNQCGASFTQKGNLLRHIKLHSGEKPFKCPFCSAGQVMSHHVPPMED

SEQ ID NO: 376 - protein, 45 aa, Homo sapiens
HKRSHTGERPFHCNQCGASFTQKGNLLRHIKLHSGEKPFKCPFCS

SEQ ID NO: 377 - protein, 14 aa, Homo sapiens
AGQVMSHHVPPMED

SEQ ID NO: 378 - protein, 4 aa, Artificial Sequence
  source: /note=“Description of Artificial Sequence: Synthetic peptide”
RTKR

SEQ ID NO: 379 - protein, 20 aa, Artificial Sequence
  source: /note=“Description of Artificial Sequence: Synthetic peptide”
GTGAEDPRPSRKRRSLGDVG

SEQ ID NO: 380 - DNA, 60 bp, Artificial Sequence
  source: /note=“Description of Artificial Sequence: Synthetic oligonucleotide”
ggaaccggcgcggaagacccccggccctccaggaagcgaaggtccctcggagacgtgggt

SEQ ID NO: 381 - protein, 14 aa, Artificial Sequence
  source: /note=“Description of Artificial Sequence: Synthetic peptide”
GTGAEDPRPSRKRR

SEQ ID NO: 382 - DNA, 42 bp, Artificial Sequence
  source: /note=“Description of Artificial Sequence: Synthetic oligonucleotide”
ggaaccggcgcggaagacccccggccctccaggaagcgaagg

SEQ ID NO: 383 - protein, 15 aa, Artificial Sequence
  source: /note=“Description of Artificial Sequence: Synthetic peptide”
LQWLEQQVAKRRTKR

SEQ ID NO: 384 - DNA, 45 bp, Artificial Sequence
  source: /note=“Description of Artificial Sequence: Synthetic oligonucleotide”
ctgcaatggctggagcagcaggtggcgaagcggagaactaagcgg

SEQ ID NO: 385 - protein, 18 aa, Artificial Sequence
  source: /note=“Description of Artificial Sequence: Synthetic peptide”
GTGAEDPRPSRKRRSLGG

SEQ ID NO: 386 - DNA, 54 bp, Artificial Sequence
  source: /note=“Description of Artificial Sequence: Synthetic oligonucleotide”
ggcacaggtgccgaggaccctcggccaagccgcaaaaggaggtcacttggcggc

SEQ ID NO: 387 - protein, 17 aa, Artificial Sequence
  source: /note=“Description of Artificial Sequence: Synthetic peptide”
GTGAEDPRPSRKRRSLG

SEQ ID NO: 388 - DNA, 51 bp, Artificial Sequence
  source: /note=“Description of Artificial Sequence: Synthetic oligonucleotide”
ggaaccggagcagaagatcccagaccaagccggaaaaggcggtccctgggt

SEQ ID NO: 389 - protein, 14 aa, Artificial Sequence
  source: /note=“Description of Artificial Sequence: Synthetic peptide”
SLNLTESHNSRKKR

SEQ ID NO: 390 - DNA, 42 bp, Artificial Sequence
  source: /note=“Description of Artificial Sequence: Synthetic oligonucleotide”
agtctcaatttgactgagtcacacaattccaggaagaaaagg

SEQ ID NO: 391 - protein, 14 aa, Artificial Sequence
  source: /note=“Description of Artificial Sequence: Synthetic peptide”
CKINGYPKRGRKRR

SEQ ID NO: 392 - DNA, 42 bp, Artificial Sequence
  source: /note=“Description of Artificial Sequence: Synthetic oligonucleotide”
tgcaagatcaacggctaccctaagaggggcagaaagcggcgg

SEQ ID NO: 393 - DNA, 12 bp, Artificial Sequence
  source: /note=“Description of Artificial Sequence: Synthetic oligonucleotide”
cgtactaaaaga

SEQ ID NO: 394 - protein, 22 aa, Artificial Sequence
  source: /note=“Description of Artificial Sequence: Synthetic peptide”
MLLLVTSLLLCELPHPAFLLIP

SEQ ID NO: 395 - DNA, 66 bp, Artificial Sequence
  source: /note=“Description of Artificial Sequence: Synthetic oligonucleotide”
atgcttctcctggtgacaagccttctgctctgtgagttaccacacccagcattcctcctgatccca

SEQ ID NO: 396 - DNA, 66 bp, Artificial Sequence
  source: /note=“Description of Artificial Sequence: Synthetic oligonucleotide”
atgctgctgctggtgaccagcctgctgctgtgcgagctgccccaccccgcctttctgctgatcccc

SEQ ID NO: 397 - DNA, 1395 bp, Artificial Sequence
  source: /note=“Description of Artificial Sequence: Synthetic polynucleotide”
gaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 398 - DNA, 1458 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctcagagactacttactaccaatcatccctcaagtctcgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 399 - DNA, 1395 bp, Artificial Sequence
  source: /note=“Description of Artificial Sequence: Synthetic polynucleotide”
gaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctcagagactacttactaccaatcatccctcaagtctcgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 400 - protein, 486 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 401 - protein, 465 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 402 - DNA, 1458 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 403 - DNA, 1395 bp, Artificial Sequence
  source: /note=“Description of Artificial Sequence: Synthetic polynucleotide”
gaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 404 - DNA, 1458 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctcagagactacttactaccaatcatccctcaagtctcgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 405 - DNA, 1395 bp, Artificial Sequence
  source: /note=“Description of Artificial Sequence: Synthetic polynucleotide”
gaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctcagagactacttactaccaatcatccctcaagtctcgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 406 - DNA, 1467 bp, Artificial Sequence
  source: /note=“Description of Artificial Sequence: Synthetic polynucleotide”
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 407 - protein, 489 aa, Artificial Sequence
  source: /note=“Description of Artificial Sequence: Synthetic polypeptide”
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 408 - DNA, 801 bp, Artificial Sequence
  source: /note=“Description of Artificial Sequence: Synthetic polynucleotide”
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctca

SEQ ID NO: 409 - protein, 266 aa, Artificial Sequence
  source: /note=“Description of Artificial Sequence: Synthetic polypeptide”
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVS

SEQ ID NO: 410 - DNA, 1401 bp, Artificial Sequence
  source: /note=“Description of Artificial Sequence: Synthetic polynucleotide”
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 411 - protein, 467 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 412 - DNA, 735 bp, Artificial Sequence
  source: /note=“Description of Artificial Sequence: Synthetic polynucleotide”
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctca

SEQ ID NO: 413 - protein, 245 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 414 - DNA, 1383 bp, Artificial Sequence
  source: /note=“Description of Artificial Sequence: Synthetic polynucleotide”
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 415 - protein, 461 aa, Artificial Sequence
  source: /note=“Description of Artificial Sequence: Synthetic polypeptide”
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 416 - DNA, 801 bp, Artificial Sequence
  source: /note=“Description of Artificial Sequence: Synthetic polynucleotide”
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 417 - protein, 267 aa, Artificial Sequence
  source: /note=“Description of Artificial Sequence: Synthetic polypeptide”
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 418 - DNA, 1317 bp, Artificial Sequence
  source: /note=“Description of Artificial Sequence: Synthetic polynucleotide”
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 419 - protein, 439 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 420 - DNA, 735 bp, Artificial Sequence
  source: /note=“Description of Artificial Sequence: Synthetic polynucleotide”
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc